(19) United States
(12) Patent Application Publication
(10) Pub. No.: US 2026/0125646 A1
(43) Pub. Date: May 7, 2026

(54) IMMUNE COMPATIBLE CELLS FOR ALLOGENEIC CELL THERAPIES TO COVER GLOBAL, ETHNIC, OR DISEASE-SPECIFIC POPULATIONS

(71) Applicant: C/O Stratus Therapeutics, Inc., Cambridge, MA (US)

(72) Inventor: Dhvanit Shah, Chestnut Hill, MA (US)

(21) Appl. No.: 19/117,837

(22) PCT Filed: Oct. 5, 2023
(86) PCT No.: PCT/US2023/076083

§ 371 Date: Apr. 2, 2025

Related U.S. Application Data
(60) Provisional application No. 63/413,331, filed on Oct. 5, 2022.

(51) Int. Cl. C12N 5/0789; A61K 35/545; C07K 14/74

(52) U.S. Cl. CPC C12N 5/0647; A61K 35/545; C07K 14/70539; C12N 2506/45; C12N 2510/00

(57) ABSTRACT

In the various aspects and embodiments, the present disclosure provides cell populations or cell “banks” thereof (e.g., cell collections) to provide immune compatible, allogeneic cell therapies covering global, ethnic, and disease-specific populations. In the various aspects and embodiments, the cell banks and progeny thereof maintain sufficient HLA Class I and HLA Class II functionalities, while facilitating patient matching to prevent or reduce graft versus host disease (GVHD) or graft rejection. The disclosure further provides methods for creating the cell banks by gene editing, and methods for cell therapy involving cells or tissues derived from the cell banks (including but not limited to hematopoietic stem cells, or “HSCs”, progenitors, or progenies thereof).

### PRIORITY

This application claims priority to, and the benefit of, U.S. provisional application No. 63/413,331 filed Oct. 5, 2022, which is hereby incorporated by reference in its entirety.

### SEQUENCE LISTING

The instant application contains a Sequence Listing which has been submitted in XML format via EFS-Web and is hereby incorporated by reference in its entirety. Said XML copy, created on Sep. 25, 2023, is named GRU-009/121145-5009_Sequence_Listing.xml and is 1,298,736 bytes in size.

### BACKGROUND

Cellular therapies, based on cells derived from allogeneic (derived from healthy donor), autologous (derived from patients), and/or induced pluripotent stem cells (iPSCs), have enormous potential for medical applications to regenerate cells and tissues. However, the use of allogeneic or autologous cells (generated from cells of an intended recipient) will not be practical in most instances. Meanwhile, transplant of cells or tissues produced from allogeneic cells face issues of immune rejection and/or Graft Versus Host Disease (GVHD), for example, caused by significant HLA mismatching. Likewise, transplant of autologous or allogeneic cells faces concerns about consistency, scalability, durability and affordability. Further, it is not feasible to prepare iPSC stocks representing enough HLA haplotypes to cover a significant portion of the population, based on current HLA matching standards. Cell banks that are HLA modified to provide “off-the-shelf” cell therapies, and which provide an ease of matching with a substantial portion of the population, and which are consistent, scalable, and of lower cost are of great need. In various aspects and embodiments, the present disclosure provides HLA-modified cells and collections thereof to meet these and other objectives.

### SUMMARY OF THE DISCLOSURE

In the various aspects and embodiments, the present disclosure provides cell populations or cell “banks” thereof (e.g., cell collections) to provide immune compatible, allogeneic cell therapies covering global, ethnic, and disease-specific populations. In the various aspects and embodiments, the cell banks and progeny thereof maintain sufficient HLA Class I and HLA Class II functionalities, while facilitating patient matching to prevent or reduce graft versus host disease (GVHD) or graft rejection. The disclosure further provides methods for creating the cell banks by gene editing, and methods for cell therapy involving cells or tissues derived from the cell banks (including but not limited to hematopoietic stem cells, or “HSCs”, progenitors, or progenies thereof).

In the various aspects and embodiments, the present disclosure provides an HLA-modified cell population that is HLA-A^{neg}, and is (1) HLA-DPA1^{neg} and/or HLA-DPB1^{neg}, and/or (2) HLA-DQA1^{neg} and/or HLA-DQB1^{neg}, the cell population being homozygous for or comprising a single gene for HLA-C, HLA-DRB1, and optionally HLA-B. In some embodiments, the cell population is HLA-A^{neg}, HLA-DPB1110g, and HLA-DQB1^{neg}, and homozygous at one or more (or all) of HLA-B, HLA-C and HLA-DRB1. In various embodiments, the cell population has a haplotype described herein.

In the various aspects and embodiments, the cell population is a human stem cell or human progenitor cell population. In some embodiments, the stem cell is a pluripotent stem cell, which may be a human induced pluripotent stem cell (hiPSC). In various embodiments the iPSCs are derived from peripheral blood CD34+ cells. In some embodiments, the stem cell population is a hematopoietic stem cell (HSC) population (e.g., differentiated from the iPSCs), or a cell population derived therefrom. In some embodiments, the cell population comprises cells that are a hematopoietic cell lineage, such as a hematopoietic lineage selected from common lymphoid precursor (CLP) cells, granulocyte-monocyte progenitor (GMP) cells, progenitor-T cells, T lymphocytes, B lymphocytes, Natural Killer cells, neutrophils, monocyte, macrophages, red cells, megakaryocytes, and platelets. In still other embodiments, the cell population is a non-hematopoietic cell population, e.g., differentiated from the iPSCs ex vivo. Exemplary cells include, but are not limited to, mesenchymal stem cell, neural stem cell, or epithelial stem cell. In some embodiments, the non-hematopoietic cell is selected from neurons, astrocytes, oligodendrocytes, cardiomyocytes, skeletal muscle cells, hepatocytes, pancreatic β cells, and lung epithelial cells, or progenitors thereof.

In other aspects, the present disclosure provides a method for cell therapy, comprising, administering to a recipient in need thereof a cell population or tissue derived from the cell population disclosed herein. In the various embodiments, the cell population is matched for the retained classical HLA. For example, in embodiments where the cell population retains HLA-B, HLA-C, and HLA-DRB1, the administered cell population or tissue is matched with the recipient at one or more (or all) of HLA-B, HLA-C and HLA-DRB1.

In other aspects, the invention provides a method for cell therapy (or uses of the cell compositions for cell therapy), comprising administering a cell population described herein, or pharmaceutically acceptable composition thereof, to a human subject in need thereof. In various embodiments, the methods described herein are used to treat blood (malignant and non-malignant), bone marrow, and immune diseases. In various embodiments, the human subject has a condition comprising one or more of lymphopenia, a cancer, infectious disease (e.g., viral disease such as HPV or HIV) an immune deficiency, an autoimmune disease, a skeletal dysplasia, hemoglobinopathies, an anemia, a bone marrow failure syndrome, and a genetic disorder (e.g., a genetic disorder impacting the immune system).

In various embodiments an HSC population is administered to the recipient, or in other embodiments, the cell population is a hematopoietic cell lineage differentiated (e.g., ex vivo) from the HSC population. In other embodiments, the cell population is a non-hematopoietic lineage differentiated from the iPSCs described herein. In the various embodiments, the subject has a condition selected from a hematological malignancy, aplastic anemia, hemoglobinopathy, inborn error of metabolism, and severe immunodeficiency. Other conditions and disorders to be treated are disclosed herein and include lymphopenia, cancer, immune deficiency, autoimmune disease, skeletal dysplasia, a bone marrow failure syndrome, and genetic disorder impacting the immune system.

In some embodiments, the subject is a tissue or organ transplant recipient. In some embodiments, the subject is experiencing or is at risk for GVHD. Organs that can be transplanted, for example, include the heart, kidneys, liver, lungs, pancreas, intestine, and thymus, among others. Tissues for transplant can include, for example, bones, tendons (both referred to as musculoskeletal grafts), bone marrow or HSCs, cornea, skin, heart valves, nerves and/or veins.

In one aspect, the present disclosure provides a method for making a cell population of the present disclosure, where the method comprises providing an iPSC population and modifying the iPSC population to prepare an HLA-modified iPSC population that is HLA-A^{neg}, and is (1) HLA-DPA1^{neg} and/or HLA-DPB1^{neg} and/or (2) HLA-DQA1^{neg} and/or HLA-DQB1^{neg}. In various embodiments, the iPSC population is homozygous for or comprises a single gene for one or more (or all) of HLA-B, HLA-C, HLA-DRB1. The method further comprises preparing embryoid bodies (EBs) from the iPSC population; dissociating the EBs and enriching for CD34-cells to prepare a CD34+-enriched cell population; and inducing endothelial-to-hematopoietic transition (EHT) of the CD34+-enriched cell population to prepare a population comprising hematopoietic stem cells (HSCs) and/or hematopoietic stem progenitor cells (HSPCs). In some embodiments, the method may further comprise harvesting CD34+ cells from the population comprising HSCs and/or HSPCs to enrich for a population undergoing EHT. In some embodiments, the method further comprises differentiating the cell population undergoing EHT to a hematopoietic lineage.

In the various embodiments, the iPSC is HLA-modified using CRISPR-Cas9, CRISPR-Cas12, STAR-CRISPR, CRISPR-CasX, CRISPR-associated transposase, zinc-finger nuclease, RNA editor, insulated genomic domain-platform editing, or combinations thereof. In the various embodiments, the iPSC is HLA-modified by electroporation with a CRISPR-Cas9 endonuclease and one or more guide RNAs (gRNAs) as ribonucleoprotein.

In various embodiments, the CD34+ enrichment and endothelial-to-hematopoietic transition is induced at Day 7 to Day 15 of iPSC differentiation. In embodiments the induction of endothelial-to-hematopoietic transition comprises increasing the expression or activity of dnmt3b, such as, but not limited to, by Piezol activation. Other methods for inducing EHT are described herein. In the various embodiments, the CD34+-enriched cells undergoing EHT are differentiated to one or more of common lymphoid precursor (CLP) cells, granulocyte-monocyte progenitor (GMP) cells, progenitor-T cells, T lymphocytes, B lymphocytes, Natural Killer cells, neutrophils, monocyte, macrophages, red cells, megakaryocytes, and platelets. In the various embodiments the CD34+-enriched cells undergoing EHT are differentiated ex vivo to progenitor T cells, T cells, or NK cells.

In various aspects, the present disclosure provides a method for making an HLA-modified cell of the present disclosure, where the method comprises contacting a cell with a Cas endonuclease and one or more guide RNAs (gRNAs) targeting the Cas endonuclease to one or more HLA-specific or HLA allele-specific regions. In the various embodiments the Cas endonuclease comprises Cas9. In the various embodiments each gRNA is a single guide RNA (sgRNA). In various embodiments, the contacting comprises electroporating the Cas endonuclease and the one or more gRNAs as ribonucleoprotein. Exemplary gRNA to target certain HLA haplotypes are described herein.

Other aspects and embodiments of this disclosure will be apparent from the following detailed disclosure and working examples.

### DESCRIPTION OF THE FIGURES

FIG. 1 shows the coverage for most frequent haplotypes (based on HLA-C, HLA-B, and DRB1) in the U.S. Two haplotypes provide cumulative coverage of about 22%, while about 50 haplotypes provide cumulative coverage of about 70% of the U.S. population.

FIG. 2 illustrates an HSC cell bank differentiated from iPSCs that are gene edited to knockout HLA genes.

FIG. 3 shows that ETV2 over-expression (OE) does not affect pluripotency. FIG. 3 shows FACS plots representative of transduction efficiency of iPSC with an adenoviral vector to overexpress ETV2 and GFP sequences. ETV2 overexpression does not affect the iPSC stemness as shown by the expression of the TRA-1-60 stemness marker.

FIG. 4 shows that ETV2 over-expression (OE) increases the yield of hemogenic endothelial cells. Representative flow cytometric analysis of hemogenic endothelial cells (described as CD235a-CD34+CD31+) and relative quantification demonstrates that ETV2-OE enhances the formation of hemogenic endothelial cells.

FIG. 5 shows that ETV2 over-expression (OE) enhances CD34+ cell formation during iPSC differentiation. Representative flow cytometric analysis of CD34+ cells and relative quantification demonstrates that ETV2-OE enhances the CD34+ cell formation.

FIG. 6A and FIG. 6B show that iPSC-derived HSCs that are derived with Piezol activation undergo pro-T cell differentiation similar to bone marrow (BM)-HSCs. FIG. 6A is a FACS plot of differentiation efficiency to CD34+CD7+ pro T cells of Bone Marrow (BM) HSCs and iPSC-HSCs derived with Piezol activation. FIG. 6B is a quantification of CD34+CD7+ cells (%) derived with (1) BM-HSCs and (2) iPSC-HSCs (Piezol Activation).

FIG. 6B shows the average of three experiments.

FIG. 7A and FIG. 7B show that iPSC-derived HSCs generated with Piezol activation undergo T cell differentiation and such T cells can be activated with CD3/CD28 beads similar to T cells derived from BM-HSCs. FIG. 7A is a FACS plot of activation efficiency (CD3+CD69+ expression) of T cells differentiated from BM-HSCs and iPSC-derived HSCs generated with Piezol activation. FIG. 7B is a quantification of CD3+CD69+ cells (%) derived with (1) BM-HSCs and (2) iPSC-HSCs (Piezol Activation). FIG. 7B shows the average of three experiments.

FIG. 8 shows that iPSC-derived HSCs can differentiate to functional T cells. IFNγ expression is a consequence of T cell activation after T cell receptor (TCR) stimulation via CD3/CD28 beads. IFNγ expression in T cells differentiated from iPSC-derived HSCs, generated upon Piezol activation, enhances HSC ability to further differentiate to functional cells (e.g., cells). FIG. 8 shows the average of three experiments.

FIG. 9A-C show: generation of three CCR5-knockout (KO) iPSC clones (A), that CCR5-KO does not affect the iPSC pluripotency (B), and that CCR5-KO does not affect the ability of cells to undergo the endothelial-to hematopoietic transition (C).

FIG. 10A-C show: generation of three CD33-KO iPSC clones (A), that CD33-KO does not affect the ability of cells to undergo the endothelial-to hematopoietic transition (B), and that CD33-KO does not affect the ability of cells to generate self-renewing HSCs (C).

FIG. 11A shows the presence of HLA genes located on the short arm of chromosome 6. FIG. 11B shows a schematic representation of targeting the HLA-A, HLA-DQB1, and HLA-DPB1 genes using gRNAs. Exons are illustrated by horizontal arrows; vertical arrows denote locations of gRNA targeting. Genomic coordinates are shown in parentheses.

FIGS. 12A and 12B show the phenotype analysis of triple knockout (HLA edited) cells performed by FACS and immunofluorescence. FIG. 12A shows the overall expression of HLA class-I molecules (HLA-A, HLA-B, and HLA-C) on the cell surface, where the HLA edited cells are positive for overall HLA class-I expression to a similar degree as wild-type cells. FIG. 12B shows cell expression of HLA-A via immunofluorescence, where HLA-A is not expressed in the HLA edited clone.

FIG. 13 shows that the HLA edited clones preserve their pluripotency (maintain trilineage differentiation), as illustrated by immunofluorescence, with ectoderm differentiation indicated by NESTIN-488 and PAX6-594 staining, mesoderm differentiation indicated by GATA-488 staining, and endoderm differentiation indicated by CXCR4-488 and FOX2A-594 staining.

FIG. 14 shows the immune compatibility of the HLA edited HSCs. HLA edited HSCs and control HSCs (WT, B2M KO, and HLA Class II null) were co-cultured with peripheral blood mononuclear cells (PBMCs) matching HLA-B and HLA-C, but with mismatched HLA-A. The PBMC-CD8+, and NK cell-mediated cytotoxicity was measured by an annexin V staining assay.

FIG. 15 shows in vivo engrafting potential of HLA edited HSCs. Equal proportions of mCherry HLA edited HSCs and wild-type HSCs were mixed for a competitive transplant into mice, where bone marrow (BM) and peripheral blood samples were evaluated by FACS to compare the relative amounts of each cell type present in the samples.

FIGS. 16A and 16B show that WT and HLA-edited HSCs can differentiate to Pro-T Cells (FIG. 16A), as identified by a combination of CD34-CD7+ and CD34+CD7+ markers. FIG. 16B graphically represents the results shown in FIG. 16A.

FIG. 17 shows that WT and HLA-edited HSCs can differentiate to the NK cell lineage, as identified by CD3-CD56+ markers.

FIGS. 18A and 18B show that WT and HLA-edited HSCs can differentiate to the monocyte/macrophage lineage, which also preserves the overall expression of both class I and class II molecules as identified by CD11b+CD14+ markers (FIG. 18A). FIG. 18B shows analysis of HLA-I and HLA-II on cells gated on CD11b+CD14+.

FIGS. 19A to 19C show that HLA-DQB1 and HLA-DPB1 deletion does not affect the expression of other HLA Class II molecules. FIG. 19A is a schematic showing differentiation of HLA-edited iPSCs to macrophages. FIG. 19B is an immunofluorescence experiment confirming the specific deletion of the DPB1 and DQB1 molecules. FIG. 19C shows that the same cells preserve the class II DRB1 expression.

FIGS. 20A and 20B show that deletion of HLA-A does not impact Class I peptide presentation. FIG. 20A shows a schematic representation of immunopeptidome analysis. FIG. 20B shows results of the immunopeptidome analysis, which reveals that little difference exists in the numbers of peptides and representative proteins presented by class I molecules of WT and HLA-edited cells.

FIGS. 21A and 21B show that deletion of HLA-DP and DQ does not impact Class II peptide presentation. FIG. 21A shows immunopeptidome analysis scheme. FIG. 21B shows that despite the deletion of HLA-DP and DQ, the cells preserve their ability to present a broad spectrum of peptide through HLA Class II.

FIG. 22 is a schematic representation of in vivo testing of antigen-mediated immune response: Delayed Type Hypersensitivity Assay (DTH), sensitizing stage and elimination stage respectively.

FIGS. 23A and 23B show that HLA-edited HSCs reconstitute a functional immune system as demonstrated by DTH reaction in immune deficient mice. FIG. 23A shows a delayed-type hypersensitivity assay on transplanted mice were performed, which is an assay that involves the cross-talk of different types of immune cells. Specifically, mice were sensitized by subcutaneous injection of sheep Red blood cells (antigen). A functional immune system results in the swelling of the left paw that was measured with a micro caliper. As can be seen in FIG. 23A, the non-transplant mice did not show any left paw swelling as they are immunodeficient. Conversely, the mice transplanted with Cord Blood CD34+ cells show tissue swelling and doubled the diameter of their left paw. FIG. 23B is a graphical evaluation of the results shown in FIG. 23A.

FIG. 24 shows HSC-derived T cells can be activated in vitro. Top panel shows FACS analysis of activated T cells from different sources, including from HSCs prepared according to the present disclosure. T cells of the present disclosure demonstrate comparable or superior activation as measured by increased CD107 expression. The lower panel shows Dynabeads activation, where activated T cells express inflammatory cytokines. HSC-derived T cells express higher levels of inflammatory cytokines as exemplified by TNF-alpha and interferon gamma expression levels.

FIG. 25 shows that CCR5-knocked out HSCs can comparably differentiate into pro-T cells, compared to their wild type (gHSC) counterpart HSC (CCR5 retained).

FIG. 26 shows CCR5-knocked out HSCs can comparably differentiate into double positive (CD4+CD8+) T cells when compared to their wild type (gHSC) counterpart HSCs (CCR5 retained).

FIG. 27 shows that HSCs generated according to this disclosure (D8+7 iPSC-CD34+ cells, with and without Yoda 1, “Y”) successfully differentiate into CD4+CD8+ (“double positive”) T cells as well as TCR α/β T cells. The methods of the present disclosure substantially outperform T cell maturation from bone marrow CD34+ cells.

FIG. 28 shows that HSCs generated according to this disclosure (D8+7 iPSC-CD34+ cells, with or without Y) successfully rearrange TCR, and outperform bone marrow CD34+ cells.

FIG. 29 shows the HSC differentiation potential into T cell subtypes. After a 35-day differentiation period pro-T cells were evaluated by cell sorting for the presence of CD4+, CD8+, and AB+ T cell populations. FIG. 29 (right) compares the differentiation potential of bone marrow-derived CD34+ cells, embryoid body CD34+ cells, and HSCs prepared according to the present disclosure (e.g., using Piezol activation).

FIG. 30 shows the degree of T-cell mediated cytotoxicity measured from a co-culture of HSC-derived T cells with CD19+ lymphoma cells in the presence of an anti-CD3/CD-19 bispecific antibody. T cells prepared from HSCs according to the present disclosure demonstrate a high level of cytotoxicity against the target cells.

FIG. 31 shows that HSC-derived T cells (pro-T cells) can be transduced with high efficiency. Pro-T cells underwent lentiviral (LV) transduction with an anti-CD-19 chimeric antigen receptor (CAR) transgene (left), where the efficiency of LV transduction was measured by cell sorting based on anti-CD19 scFv staining (right). Results indicate that HSC-derived T cells achieved approx. 85% transduction efficiency.

FIG. 32 shows that LV-transduced HSC-derived T cells (pro-T cells) can effectively mature into CD4+/CD8+ T cells via CAR transduction.

FIG. 33 shows the ability of anti-CD19 CAR-transduced HSC-derived T cells (CAR pro-T cells) to function via receptor-mediated cytotoxicity. Luc+ NALM6 leukemia cells were co-cultured with CAR pro-T cells and cell-mediated cytotoxicity was measured by luciferase assay.

FIG. 34 shows the ability of the HSCs to develop into pro-T cells as measured by their CD34-CD7+ markers.

FIGS. 35A and 35B demonstrates increased expression of T cell-specific transcription factors and Thymus engrafting molecules with the pro-T cells derived from HSCs according to the instant disclosure. FIG. 35A shows TCF7 mRNA expression and FIG. 35B shows CCR7 mRNA expression.

FIGS. 36A and 36B shows that HSC-derived Pro-T Cells engraft and differentiate in thymus. FIG. 36A illustrates the engraftment and analysis procedure. FIG. 36B shows FACS analysis of CD3 cell population of cells gated on CD45+ cell population, which shows the superior engraftment and differentiation potential of the HSC-derived Pro-T Cells in the thymus.

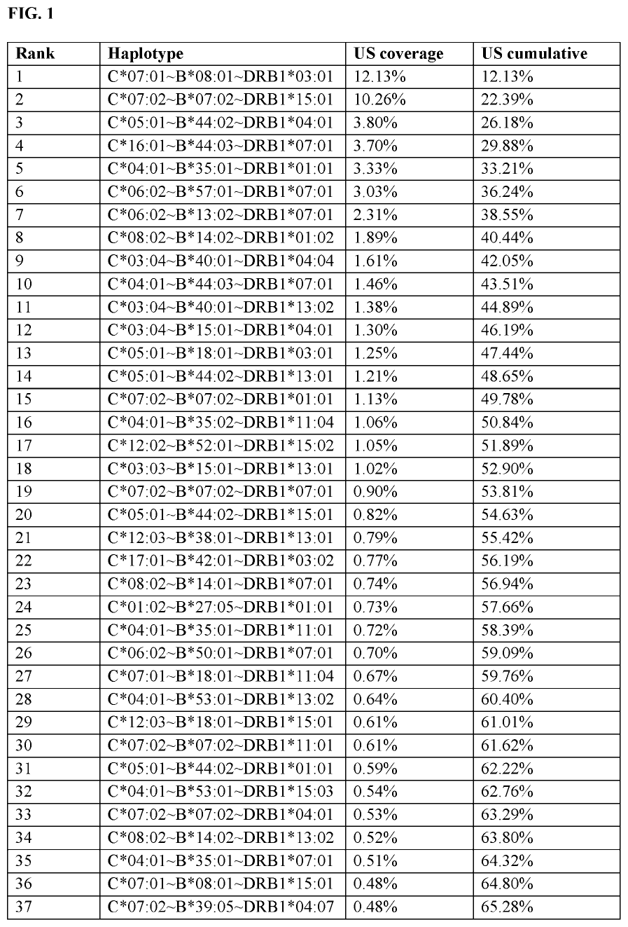

The term “gHSC” is used herein to refer to the iPSC-derived hematopoietic stem cells of the present disclosure.

The terms “wild type” (WT), “unedited”, “non-HLA-edited” are used interchangeability herein to refer to the non-gene edited cells of the present disclosure.

EB34+ cells refer to Embryonic body derived CD34+ cells. These comprise hemogenic endothelial cells.

### DETAILED DESCRIPTION

In the various aspects and embodiments, the present disclosure provides cell populations or cell “banks” and collections thereof to provide immune compatible, allogeneic cell therapies covering global, ethnic, and disease-specific populations. In the various aspects and embodiments, the cell banks and progeny thereof maintain sufficient HLA Class I and HLA Class II functionalities, while facilitating patient matching to prevent or reduce graft versus host disease (GVHD) or graft rejection. The disclosure further provides methods for creating the cell banks by gene editing, and methods for cell therapy involving cells or tissues derived from the cell banks (including but not limited to hematopoietic stem cells, or “HSCs”, as well as progenitors and progenies thereof).

In an aspect, the disclosure provides an HLA-modified cell population that is HLA-A^{neg}, and is (1) HLA-DPA1^{neg} and/or HLA-DPB1^{neg} and/or (2) HLA-DQA1^{neg} and/or HLA-DQB1^{neg}. Further, the cell population is homozygous for, or comprises a single gene for, HLA-C, HLA-DRB1, and optionally HLA-B.

In some embodiments, the cell population is HLA-DPB1^{neg} and HLA-DQB1^{neg} In some embodiments, the cell population comprises a deletion or inactivation of both DPB1 genes and both DQB1 genes.

In some embodiments, one or both DQB1 genes are deleted or are retained. In some embodiments, one or both DQA1 genes are retained. In some embodiments, the cell population retains DQB1 unmodified. In other embodiments, the cell population comprises a deletion or inactivation of both DQB1 genes, and optionally of both DQA1 genes. In these embodiments, the cell population is HLA-DQ^{neg}.

In some embodiments, one or both DPB1 genes are deleted or are retained. In some embodiments, one or both DPA1 genes are retained. Thus, the cell population may lack both copies or retain HLA-DP, and which can be a single copy (one copy deleted or inactivated), or can retain HLA-DP as unmodified.

In some embodiments, the cell population comprises a deletion or inactivation of both DPB1 genes and both DQB1 genes. In these embodiments, DRB1 is retained, and is either homozygous or single copy, to support HLA-Class II function.

In various embodiments, the cell population is homozygous or comprises a single copy of HLA-B. Alternatively, the cell population may comprise a deletion or inactivation of both HLA-B genes. In these embodiments, Class I antigen presentation may be predominately supported by HLA-C.

In various embodiments, the cell population retains one copy of HLA-DRB1, and another copy of HLA-DRB1 is deleted or inactivated. In some embodiments, the cell population is homozygous or has only a single copy of one, two, three, or four of DRB2, DRB3, DRB4, and DRB5. Optionally, DRB2, DRB3, DRB4, and DRB5 are retained and unmodified.

In some embodiments, the cell population is homozygous at HLA-E or one HLA-E gene is deleted or inactivated. In some embodiments, HLA-E is unmodified, and may be homozygous or heterozygous.

In some embodiments, the cell population is homozygous at HLA-F or one HLA-F gene is deleted or inactivated. In some embodiments, HLA-F is unmodified, and may be homozygous or heterozygous.

In some embodiments, the cell population is homozygous at HLA-G or one HLA-G gene is deleted or inactivated. In some embodiments, HLA-G is unmodified, and may be homozygous or heterozygous.

In certain embodiments, the cell population is HLA-A^{neg}, homozygous for both HLA-B and HLA-C, and is HLA-DPB1^{neg} and HLA-DQB1^{neg}. In some embodiments, the cell population is further homozygous for HLA-DRB1.

In various embodiments, the cell population is a stem cell population, such as a pluripotent stem cell. In some embodiments, the cell population is a human induced pluripotent stem cell (hiPSC). As described in further detail herein, iPSCs may be derived from cord blood, bone marrow biopsy, mobilized peripheral blood derived hCD34+ cells, human CD34+ cells, immune cells, immune progenitor cells, hematopoietic cells, non-hematopoietic cells (e.g., cells that can differentiate into cells such as fibroblasts, osteoblasts, chondrocytes, myocytes, endothelial cells, and neurons), and banked organ derived cells. In various embodiments, as described further below, primary cells are reprogrammed to generate human iPSC cell bank(s), which can be HLA-modified to generate off-the-shelf therapeutics containing immune compatible, allogeneic human cells.

In some embodiments, the stem cell population is a hematopoietic stem cell (HSC) population or a hematopoietic stem progenitor cell (HSPC) population, or a cell population derived therefrom. As described in further detail herein, the cell population may be, or may be used to derive, a hematopoietic cell lineage. For example, the hematopoietic lineage may be selected from common lymphoid precursor (CLP) cells, granulocyte-monocyte progenitor (GMP) cells, progenitor-T cells, T lymphocytes, B lymphocytes, Natural Killer cells, neutrophils, monocyte, macrophages, dendritic cells, red cells, megakaryocytes, and platelets.

In some embodiments, the cell population may be human donor- or patient-derived cells, including but not limited to donor- or patient-derived HSCs, hematopoietic progenitors, and hematopoietic lineages (e.g., selected from CLP cells, GMP cells, progenitor-T cells, T lymphocytes, B lymphocytes, Natural Killer cells, neutrophils, monocyte, macrophages, dendritic cells red cells, megakaryocytes, and platelets).

In still other embodiments, the cell population is a non-hematopoietic stem cell population. The population can be derived from iPSCs, or may be donor or patient derived. Exemplary non-hematopoietic stem cells include mesenchymal stem cell, neural stem cell, or epithelial stem cell. In still other embodiments, the cell population is, or is used to derive, a non-hematopoietic cell, such as a cell selected from fibroblasts, osteoclasts, chondrocytes, myocytes, cardiomyocytes, endothelial cells, neurons, astrocytes, oligodendrocytes, hepatocytes, pancreatic β cells, and lung epithelial cells, or progenitors thereof.

In some embodiments, the cell population(s) are stem cells or progenitor cells used to generate hCD34+ HSCs, hematopoietic progenitors, immune cells, platelets, red cells, other hematopoietic precursors, and lineages, as well as non-hematopoietic cells, including but not limited to neurons, astrocytes, oligodendrocytes, myocytes, cardiomyocytes, hepatocytes, pancreatic β cells, lung epithelial cells, etc.

According to aspects and embodiments of this disclosure, the cell population is a pluripotent stem cell (e.g., iPSC)-derived hematopoietic progenitor, such as hematopoietic stem cells (HSCs), common myeloid progenitors (CMPs) or common lymphoid progenitors (CLP), which can give rise to progenies, such as, red blood cells/erythrocytes, platelets, mast cells, osteoclasts, granulocytes, monocyte-macrophages, dendritic cells, T-cells/T-lymphocytes, B-cells/B-lymphocytes, NK-cells/natural killer cells, and dendritic cells.

In various aspects and embodiment of this disclosure, the cell populations and cell banks (whether allogeneic, autologous, or iPSC-derived cells) are developed according to HLA haplotype or polymorph distributions based on region, ethnicity, and/or target disease. In embodiments, cell populations or cell lines are developed using suitable primary cell donors according to rank orders of common or uncommon HLA haplotypes to cover a target population. For example, the cumulative addition of cell lines to the bank can be based on HLA haplotypes and/or polymorphs to cover heterogenous US population, heterogenous Caucasian population, heterogenous Beta-Thalassemia population, or heterogenous global population (or other population). In various embodiments, the cumulative addition of cell lines to the bank covers core HLA haplotypes and/or polymorphs in a heterogeneous Asian population, heterogeneous European population, heterogeneous African population, heterogeneous South American population, or heterogeneous North American population.

In various embodiments, the cell population has a DRB1 haplotype selected from DRB1*03:01, DRB1*15:01, DRB1*01.02, DRB1*07.01, DRB1*04:01, DRB1*07:01, DRB1*01:01, DRB1*01:02, DRB1*04:04, DRB1*13:02, DRB1*13:01, DRB1*11:04, and/or DRB1*15:02, DRB1*03:02, DRB1*11:01, DRB1*15:03, DRB1*04:07, DRB1*08:04, DRB1*04:02, DRB1*14:01, and DRB1*10:01.

In some embodiments, the cell population has an HLA-C haplotype selected from C*07:01, C*04:01, C*07:02, C*06:02, C*03:04, C*05:01, C*02:02, C*12:03, C*03:03, and C*16:01.

In some embodiments, the cell population has an HLA-B haplotype selected from B*08:01 and B*07:02. For example, the cell population has an HLA-B haplotype selected from B*08:01, B*07:02, B*44:02, B*44:03, B*35:01, B*57:01, B*15:01, B*14:02, B*40:01, B*53:01, B*49:01, B*51:01, B*13:02, and B*38:01.

In some embodiments, the cell population comprises an HLA-C˜HLA-B˜DRB1 haplotype selected from:

| | |
|---|---|
| C*07:01~B*08:01~DRB1*03:01, | C*07:02~B*07:02~DRB1*15:01, |
| C*05:01~B*44:02~DRB1*04:01, | C*16:01~B*44:03~DRB1*07:01, |
| C*04:01~B*35:01~DRB1*01:01, | C*06:02~B*57:01~DRB1*07:01, |
| C*06:02~B*13:02~DRB1*07:01, | C*08:02~B*14:02~DRB1*01:02, |
| C*3:04~B*40:01~DRB1*04:04, | C*04:01~B*44:03~DRB1*07:01, |
| C*03:04~B*40:01~DRB1*13:02, | C*03:04~B*15:01~DRB1*04:01, |
| C*05:01~B*18:01~DRB1*03:01, | C*05:01~B*44:02~DRB1*13:01, |
| C*07:02~B*07:02~DRB1*01:01, | C*04:01~B*35:02~DRB1*11:04, |
| C*12:02~B52:01~DRB1*15:02, | C*03:03~B*15:01~DRB1*13:01, |
| C*07:02~B*07:02~DRB1*07:01, | C*05:01~B*44:02~DRB1*15:01, |
| C*12:03~B*38:01~DRB1*13:01, | C*17:01~B*42:01~DRB1*03:02, |
| C*08:02~B*14:01~DRB1*07:01, | C*01:02~B*27:05~DRB1*01:01, |
| C*04:01~B*35:01~DRB1*11:01, | C*06:02~B*50:01~DRB1*07:01, |
| C*07:01~B*18:01~DRB1*11:04, | C*04:01~B*53:01~DRB1~*13:02, |
| C*12:03~B*18:01~DRB1*15:01, | C*07:02~B*07:02~DRB1*11:01, |
| C*05:01~B*44:02~DRB1*01:01, | C*04:01~B*53:01~DRB1*15:03, |
| C*07:02~B*07:02~DRB1*04:01, | C*08:02~B*14:02~DRB1*13:02, |
| C*04:01~B*35:01~DRB1*07:01, | C*07:01~B*08:01~DRB1*15:01, |
| C*07:02~B*39:05~DRB1*04:07, | C*04:01~B*53:01~DRB1*08:04, |
| C*07:02~B*07:02~DRB1*13:01, | C*12:03~B*38:01~DRB1*04:02, |
| C*03:03~B*15:01~DRB1*04:01, | C*04:01~B*35:01~DRB1*14:01, |
| C*06:02~B*37:01~DRB1*10:01, | C*07:01~B*49:01~DRB1*11:01, |
| C*03:04~B*40:01~DRB1*04:01, | C*15:02~B*51:01~DRB1*11:01, |
| C*03:02~B*58:01~DRB1*03:01, | C*03:03~B*55:01~DRB1*14:01, and |
| C*04:01~B*35:01~DRB1*13:01. | |

In accordance with the various embodiments, the cell line is immune compatible at two, four, six, eight, ten, or twelve HLA loci by either matching at certain HLA haplotypes or not mismatching at certain HLA haplotypes.

For example, the cell line is immune compatible at HLA-C by virtue that the cell line is homozygous at HLA-C (and HLA-C is matched), or one copy of HLA-C is matched and another copy of HLA-C is deleted or inactivated. The cell line is immune compatible at HLA-A by virtue that both HLA-A genes are deleted or inactivated (i.e., the cell line is HLA-A^{neg}). The cell line is also immune compatible at HLA-DRB1 by virtue that the cell line is homozygous at HLA-DRB1 (and thus HLA-DRB1 is matched), or one copy of HLA-DRB1 is matched and another copy of HLA-DRB1 is deleted or inactivated.

In various embodiments, the cell lines is immune compatible at HLA-B by virtue that the cell line is homozygous at HLA-B, or one copy of HLA-B is matched and another copy of HLA-B is deleted or inactivated. In some embodiments, the cell line entirely lacks HLA-B.

In some embodiments the cell lines is immune compatible at HLA-DPB1, because both copies of DPB1 are deleted or inactivated (HLA-DPB1^{neg}). Alternatively, the cell line is homozygous at HLA-DPB1 (and DPB1 is matched or unmatched), or one copy of HLA-DPB1 is matched and another copy of HLA-DPB1 is deleted or inactivated.

In some embodiments the cell lines is immune compatible at HLA-DQB1, because both copies of DQB1 are deleted or inactivated (HLA-QPB1^{neg}). Alternatively, the cell line is homozygous at HLA-DQB1 (and DQB1 is matched or unmatched), or one copy of HLA-DQB1 is matched and another copy of HLA-DQB1 is deleted or inactivated.

The cell line can be immune compatible at HLA-E by virtue that the cell line is homozygous at HLA-E, or one copy of HLA-E is matched and another copy of HLA-E is deleted or inactivated. However, in some embodiments HLA-E is retained as unmodified, and is either matched or not matched.

In some embodiments, the cell line is developed by deleting or inactivating specific HLA haplotypes using gene editing techniques, including but not limited to CRISPR-Cas9, while preserving other HLA haplotypes. For example, cell lines can be derived from human primary cells from a homozygous donor (at one or more loci), and/or by deleting one copy of mismatched haplotype. Non limiting examples of sgRNA for use with CRISPR-Cas9 gene editing systems are described herein. The sgRNAs can be used singly, or in combinations to induce gene edits, such as double strand breaks, in exon 1 and/or exon 2 of the target HLA, leading to inactivation, mutagenesis, or deletions of one base or more, such as 5 bases or more, or 10 bases or more, or 50 bases or more, or 100 bases or more, or 500 bases or more, sufficient to functionally inactivate the target gene or eliminate its functional expression. In some embodiments, the gRNA targeting domains are 16, 17, 18, 19, 20, 21, 22, 23, 24, 25, 26 or more nucleotides in length. In some embodiments, the gRNAs comprise a modification at or near the 5′ end (e.g., within 1-10, 1-5, or 1-2 nucleotides of the 5′ end) and/or a modification at or near the 3′ end (e.g., within 1-10, 1-5, or 1-2 nucleotides of the 3′ end). In some embodiments, the modified gRNAs exhibit increased resistance to nucleases. In some embodiments, a gRNA comprises two separate RNA molecules (i.e., a “dual gRNA”). A dual gRNA comprises two separate RNA molecules: a “crispr RNA” (or “crRNA”) and a “tracr RNA” and is well known to one of skill in the art.

The cell lines comprise one or more HLA modifications (e.g., one or more HLA gene deletions) to facilitate HLA matching with a recipient, to make cell therapies available to a diverse population with a universal collection of HLA matching cell lines (i.e., as compared to a non-HLA-modified collection encumbered by enormous diversity of HLA haplotypes in a population). In an aspect, the disclosure provides a collection of cell lines (or “cell populations”) comprising at least two cell lines, where the cell lines in the collection represent at least two different HLA haplotypes. For example, each cell line comprises a deletion or inactivation of HLA-A gene in addition to one or more of (1) HLA-DQA1 and/or HLA-DQB1 and (2) HLA-DPA1 and/or HLA-DPB1. Other HLA modifications to Class I and/or Class II genes are made according to the present disclosure to facilitate immune-compatibility matching with a recipient without compromising the safety or efficacy of the cell therapy.

The Major Histocompatibility complex (MHC) system, also referred to herein as human leukocyte antigen (HLA), is comprised of a polymorphic gene cluster located on the short arm of chromosome 6 (6p21.3). HLA includes regions designated as class I and class II. The main function of HLA class I gene products is to present endogenous (i.e., intracellular) peptides to cognate CD8+ (cytotoxic) T Cells. The main function of HLA class II molecules is to present peptide antigens from exogenous proteins to CD4+ helper T Cells. HLA class I gene products are critical for detecting and targeting cells that develop deleterious mutations and/or cancers, as well as for detecting and targeting cells harboring intracellular pathogens including viruses. HLA class II gene products are critical for detecting the presence of pathogens in a tissue environment and coordinating an immune response against the pathogen. While HLA class I gene products are expressed on most cells, HLA class II genes are largely expressed by professional antigen presenting cells such as dendritic cells, macrophages, and B cells. HLA class II molecules are also known to be expressed by some T cells as well as subsets of epithelial and endothelial cells, for example. Kambayashi and Laufer, Atypical MHC class II-expressing antigen-presenting cells: can anything replace a dendritic cell? Nature Reviews Immunology vol. 14:719-730 (2014).

HLA class I molecules comprise HLA-A, HLA-B, HLA-C, HLA-E, HLA-F, and HLA-G, which differ substantially in their level of polymorphism. HLA class I molecules are comprised of a single polypeptide complexed with B2-microglobulin (B2M). Indeed, knock out of B2M can abolish functional expression of HLA-class I gene products. There are about 7,453 identified HLA-A alleles, about 8,849 identified HLA-B alleles, about 7,393 identified HLA-C alleles, about 310 identified HLA-E alleles, about 50 identified HLA-F alleles, and about 102 identified HLA-G alleles. See hla.alleles.org. Natural killer (NK) cells recognize cells lacking HLA class I expression, a phenomenon often observed in a wide spectrum of tumor types. Malmberg K., Immune selection during tumor checkpoint inhibition therapy paves way for NK-cell “missing self” recognition, Immunogeneties vol. 69, pages 547-556 (2017). Generally, HLA-A and HLA-B exhibit the highest expression among class I molecules.

HLA class II molecules comprise two transmembrane polypeptide chains (a and B) forming the antigen binding cleft. HLA molecules corresponding to class II include HLA-DP, HLA-DM, HLA-DOA, HLA-DOB, HLA-DQ, and HLA-DR, and which have highly varying levels of polymorphism (see hla.alleles.org). HLA class II genes include those with “classical” class II alpha and beta chain genes of HLA-DP, -DQ and -DR, and “non-classical”loci such as HLA-DM and -DO. DRB1 shows the highest diversity among class II genes and is highly expressed.

TABLE 1
below summarizes HLA class I and class II genes.

| HGNC ID | Symbol | Name | Chromosome |
|---|---|---|---|
| HGNC: 4931 | HLA-A | major histocompatibility complex, class I, A | 6p22.1 |
| HGNC: 4932 | HLA-B | major histocompatibility complex, class I, B | 6p21.33 |
| HGNC: 4933 | HLA-C | major histocompatibility complex, class I, C | 6p21.33 |
| HGNC: 4934 | HLA-DMA | major histocompatibility complex, class II, DM alpha | 6p21.32 |
| HGNC: 4935 | HLA-DMB | major histocompatibility complex, class II, DM beta | 6p21.32 |
| HGNC: 4936 | HLA-DOA | major histocompatibility complex, class II, DO alpha | 6p21.32 |
| HGNC: 4937 | HLA-DOB | major histocompatibility complex, class II, DO beta | 6p21.32 |
| HGNC: 4938 | HLA-DPA1 | major histocompatibility complex, class II, DP alpha 1 | 6p21.32 |
| HGNC: 4939 | HLA-DPA2 | major histocompatibility complex, class II, DP alpha 2 | 6p21.32 |
| | | (pseudogene) | |
| HGNC: 19393 | HLA-DPA3 | major histocompatibility complex, class II, DP alpha 3 | 6p21.32 |
| | | (pseudogene) | |
| HGNC: 4940 | HLA-DPB1 | major histocompatibility complex, class II, DP beta 1 | 6p21.32 |
| HGNC: 4941 | HLA-DPB2 | major histocompatibility complex, class II, DP beta 2 | 6p21.32 |
| | | (pseudogene) | |
| HGNC: 4942 | HLA-DQA1 | major histocompatibility complex, class II, DQ alpha 1 | 6p21.32 |
| HGNC: 4943 | HLA-DQA2 | major histocompatibility complex, class II, DQ alpha 2 | 6p21.32 |
| HGNC: 4944 | HLA-DQB1 | major histocompatibility complex, class II, DQ beta 1 | 6p21.32 |
| HGNC: 4945 | HLA-DQB2 | major histocompatibility complex, class II, DQ beta 2 | 6p21.32 |
| HGNC: 4946 | HLA-DQB3 | major histocompatibility complex, class II, DQ beta 3 | 6p21.3 |
| HGNC: 4947 | HLA-DRA | major histocompatibility complex, class II, DR alpha | 6p21.32 |
| HGNC: 4948 | HLA-DRB1 | major histocompatibility complex, class II, DR beta 1 | 6p21.32 |
| HGNC: 4950 | HLA-DRB2 | major histocompatibility complex, class II, DR beta 2 | 6p21.3 alternate |
| | | (pseudogene) | reference locus |
| HGNC: 4951 | HLA-DRB3 | major histocompatibility complex, class II, DR beta 3 | 6p21.3 alternate |
| | | | reference locus |
| HGNC: 4952 | HLA-DRB4 | major histocompatibility complex, class II, DR beta 4 | 6p21.3 alternate |
| | | | reference locus |
| HGNC: 4953 | HLA-DRB5 | major histocompatibility complex, class II, DR beta 5 | 6p21.32 |
| HGNC: 4954 | HLA-DRB6 | major histocompatibility complex, class II, DR beta 6 | 6p21.32 |
| | | (pseudogene) | |
| HGNC: 4955 | HLA-DRB7 | major histocompatibility complex, class II, DR beta 7 | 6p21.3 alternate |
| | | (pseudogene) | reference locus |
| HGNC: 4956 | HLA-DRB8 | major histocompatibility complex, class II, DR beta 8 | 6p21.3 alternate |
| | | (pseudogene) | reference locus |
| HGNC: 4957 | HLA-DRB9 | major histocompatibility complex, class II, DR beta 9 | 6p21.32 |
| | | (pseudogene) | |
| HGNC: 4962 | HLA-E | major histocompatibility complex, class I, E | 6p22.1 |
| HGNC: 4963 | HLA-F | major histocompatibility complex, class I, F | 6p22.1 |
| HGNC: 4964 | HLA-G | major histocompatibility complex, class I, G | 6p22.1 |
| HGNC: 4965 | HLA-H | major histocompatibility complex, class I, H | 6p22.1 |
| | | (pseudogene) | |
| HGNC: 4967 | HLA-J | major histocompatibility complex, class I, J | 6p22.1 |
| | | (pseudogene) | |
| HGNC: 4969 | HLA-K | major histocompatibility complex, class I, K | 6p22.1 |
| | | (pseudogene) | |
| HGNC: 4970 | HLA-L | major histocompatibility complex, class I, L | 6p22.1 |
| | | (pseudogene) | |
| HGNC: 19406 | HLA-N | major histocompatibility complex, class I, N | 6p22.1 |
| | | (pseudogene) | |
| HGNC: 21196 | HLA-P | major histocompatibility complex, class I, P | 6p22.1 |
| | | (pseudogene) | |
| HGNC: 19395 | HLA-S | major histocompatibility complex, class I, S | 6p21.33 |
| | | (pseudogene) | |
| HGNC: 23478 | HLA-T | major histocompatibility complex, class I, T | 6p22.1 |
| | | (pseudogene) | |
| HGNC: 23477 | HLA-U | major histocompatibility complex, class I, U | 6p22.1 |
| | | (pseudogene) | |
| HGNC: 23482 | HLA-V | major histocompatibility complex, class I, V | 6p22.1 |
| | | (pseudogene) | |
| HGNC: 23425 | HLA-W | major histocompatibility complex, class I, W | 6p22.1 |
| | | (pseudogene) | |
| HGNC: 19385 | HLA-X | major histocompatibility complex, class I, X | 6p21.3 |
| | | (pseudogene) | |
| HGNC: 33913 | HLA-Y | major histocompatibility complex, class I, Y | 6p21.33 |
| | | (pseudogene) | |
| HGNC: 19394 | HLA-Z | major histocompatibility complex, class I, Z | 6p21.32 |
| | | (pseudogene) | |

For transplantation of organs and tissues (including hematopoietic stem cells) from allogeneic donors, the main criterion for donor selection is HLA compatibility. Particularly for HSC transplantation, a new lympho-hematopoietic system must develop in the recipient to replace the recipient's diseased lympho-hematopoietic system. Immunological reactions are substantially driven by T cells and include host-versus-graft (HVG) responses, which refers to patient cell reactivity against donor cells, and graft-versus-host (GVH) responses, which refers to donor lymphocyte reactivity against host tissues. The immunotherapeutic effect on neoplastic cells is often referred to as graft-versus-leukemia (GVL). GVH (or GVHD) can be associated with severe side effects in transplant recipients (e.g., HSCT) and is largely responsible for HSC transplant-related morbidity and mortality.

Molecular HLA typing conventionally involves typing the α1 and α2 domains for class I and the α1 domain for class II. Donors are generally selected based on typing of the classical HLA genes HLA-A, HLA-B, HLA-C, HLA-DRB1, HLA-DQB1, and HLA-DPB1. For example, US standards conventionally attempt to match 8 loci (both alleles for HLA-A, HLA-B, HLA-C, and HLA-DRB1), while European standards involve matching 10 loci (both alleles for HLA-A, HLA-B, HLA-C, HLA-DRB1, and HLA-DQB1).

In an aspect, the present disclosure provides a method of generating off-the-shelf cell populations or “banks” of cells. The method comprises: (i) providing donor or patient-derived cells and/or pluripotent stem cells (e.g., iPSCs); and (ii) modifying in vitro one or more endogenous coding sequences in the cell (e.g., iPSCs) genome, thereby knocking out one or more genes or mutating one or more genes to encode a nonfunctional protein in the cell population (e.g., iPSCs). The modified cells in various embodiments are identified as one of: (i) HLA-A⁻B⁺C⁺DP⁻DR⁺DQ⁺, (ii) HLA-A⁻B⁺C⁺DP⁻DR⁺DQ⁺, (iii) HLA-A⁻B⁻C⁺DP⁻DR⁺DQ⁻; (iv) HLA-A⁻B⁻C⁺DP⁻DR⁺DQ⁺; (v) HLA-A⁻B⁻C⁺DP⁺DR⁺DQ⁻, (vi) HLA-A⁻B⁻C⁺DP⁻DR⁺DQ⁻. For retained HLA (for example, HLA-B, HLA-C, and HLA-DR), cells can be homozygous or retain only a single copy of the gene. For example, the modified cells are identified at least as (a) HLA-C+ and HLA-DR+, and optionally identified as one or more of (b) HLA-B−, (c) HLA-DP−, and (d) HLA-DQ−. In exemplary embodiments, the modified cells are HLA-B+, HLA-DP−, and HLA-DQ−.

As used herein, the term “neg” or (−) with respect to a particular HLA Class I or Class II gene indicates that both copies of the gene have been disrupted in the cell line or population, and thus the cell line or population does not display significant functional expression of the gene. Such cells can be generated by full or partial gene deletions, or alternatively with other technologies such as siRNA.

In accordance with aspects and embodiments of this disclosure, cell populations or cell banks are provided to allow for harvest from allogeneic donor or patient or for the generation (e.g., by ex vivo expansion or differentiation) of hematopoietic stem cells (HSCs) and progenitors or progenies thereof for off-the shelf cell and tissue therapies. The cells are gene edited to delete particular HLA genes (as described), to thereby facilitate immune compatible matching for an intended recipient. As used herein, the term “delete” in this context refers to a genetic modification of the target gene (i.e., gene edit) that abrogates functional expression of the corresponding gene product (i.e., the corresponding polypeptide). Such gene edits include full or partial gene deletions, or deletions of critical cis-acting expression control sequences. In accordance with embodiments of this disclosure, expression of B2M is not altered, and expression of class II major histocompatibility complex transactivator (CIITA) is not altered, since these modifications would abolish HLA expression. B2M expression is critical for functional expression of HLA class I, and CIITA is critical for HLA class II expression. For example, alteration of B2M risks a response by natural killer (NK) cells and is potentially harmful for the proliferation of cells that are infected by pathogens or are oncogenic. Alteration of CIITA risks lack of antigen-presentation abilities though class II HLAs. For HSC transplantation (for example), functional class I and class II expression is required to reconstitute immunological surveillance.

In various aspects and embodiments the present disclosure provides HLA class I and/or HLA class II modified cells in which certain HLA gene(s) (as described) have been altered or deleted to make the cells immune compatible for the cell-based therapy, in a subject in need of such therapy, without being encumbered by concerns of the harmful effects of HLA incompatibility or donor matching. Advantageously, these alterations in the class I and class II molecules enhance the biocompatibility of these cells in diverse populations originating from Asia, Europe, Africa, South America, and North America as they manifest all of the characteristics of unmodified cells, except that they advantageously eliminate or ameliorate harmful or toxic functions in therapeutical applications of their HLA-unmodified counterpart cells.

In various aspects and embodiments, this disclosure minimizes the HLA loci required for haplotype matching, including for HSC transplantation. In various aspects and embodiments, the cells, or cells or tissues derived therefrom, exhibit functional class II antigen presentation (i.e., class II antigen presentation is not substantially impaired by lower class II expression and/or class II diversity in comparison to non-HLA modified cells or tissues). In various aspects and embodiments, the cells, or cells or tissues derived therefrom, do not show substantial susceptibility to oncogenesis or viral infection (i.e., due to loss of class I expression or class I diversity). In various aspects and embodiments, the cells, or cells or tissues derived therefrom, are not substantially targeted by the innate immune system of the recipient (e.g., NK cells) due to loss of HLA expression or diversity (i.e., as compared to non-HLA modified cells or tissues).

HLA haplotypes are indicated herein according to convention. HLA alleles can be named by indicating the locus, antigenic specificity, and molecularly typed allele group. The asterisk “*” sign indicates that typing is performed by a molecular method and the colon “.” is a field separator. For example, where A*03:01 is an allele of interest, the first field (A*03) refers to a group of alleles that encode for the A3 antigen, and the second field (:01) refers to a particular allele that encodes the unique HLA protein A*03:01. Homozygous alleles can include one or more polymorphisms in one or both copies in some embodiments (that is, need not be identical).

The cell populations or banks can be modified for one or more additional functionalities (as described in more detail herein), including deletion or insertion of additional genes. For example, the cell lines can be modified to express or overexpress certain cytokines, suicide genes, T-cell receptor, one or more chimeric antigen receptors (CARs), and/or combinations thereof. In some embodiments, cell populations or banks, or progeny thereof, are modified such that certain endogenously expressed genes are deleted, inactivated, or reduced in expression, such as but not limited to genes encoding CCR5 or miR-155, or genes encoding cell surface markers including but not limited to CD33, CLL, CD19, CD7, and/or CD38.

In various embodiments, cells are inserted with a nucleic acid encoding a CAR specific to myeloma, leukemia or lymphoma targets, including but not limited to CD19, CD33, and BCMA. In various embodiments, the cells are introduced with nucleic acid encoding tandem CARs, including but not limited CD38/IL3 and CD20/CD19. In various embodiments, the cells are introduced with nucleic acid encoding disease specific dual CAR, Quad CAR, or tandem-CARs,

Thus, non-limiting examples include but are not limited to: (i) cells deleted for CCR5 to generate CCR5-deleted cellular therapies of HIV-AIDS patients; (ii) cells deleted for CD33 to generate CD33-deleted cellular therapies for treating leukemia and/or lymphoma patients; (iii) cells deleted for CD33 to generate CD33-deleted cellular therapies for use in connection with CAR-T, CAR-NK, CAR-T progenitor cells, or CAR-macrophage cells for treating leukemia and/or lymphoma patients.

The cell populations, or cells derived therefrom (e.g., progeny) can be used with FDA approved CAR-T therapy, such as, Tisagenlecleucel, also known as tisa-cel (Kymriah), Axicabtagene ciloleucel, also known as axi-cel (Yescarta), Brexucabtagene autoleucel, also known as brexu-cel (Tecartus), Lisocabtagene maraleucel, also known as liso-cel (Breyanzi), Idecabtagene vicleucel, also known as ide-cel (Abecma), Ciltacabtegene autoleucel, also known as cilta-cel (Carvykti) or any other CAR-T therapy which damage the normal cells during their therapeutic applications.

In one aspect, the disclosure provides a collection of cell populations (i.e., cell banks comprising at least two populations of cells) of expanded primary cells, derivatives of iPSC cells, or stem cell lines, where the cell lines in the collection represent at least two different HLA-C haplotypes. Each cell population comprises a deletion or inactivation of both HLA-A genes. In various embodiments, the cell populations in the collection represent at least four, or at least six, or at least eight, or at least ten different HLA-C haplotypes. In some embodiments, the cell populations in the collection represent at least twelve different HLA-C haplotypes or at least fifteen or at least twenty different HLA-C haplotypes. In various embodiments, the cell populations are either homozygous for the HLA-C gene or are edited to have only a single HLA-C gene (e.g., by deletion of one HLA-C gene).

In certain embodiments, the cell populations or banks are represented by at least an (i) HLA-C allele and (ii) a class II DRB allele.

In some embodiments, the collection comprises cell populations or banks comprising one or more of the following HLA-C alleles selected from: C*01, C*02, C*03, 7 C*04, C*05, C*06, C*07, C*08, C*12, C*14, C*15, C*16, C*17, C*18. In some embodiments, these HLA-C alleles are matched with DRB alleles selected from DRB 1*01, DRB 1*03, DRB 1*04, DRB 1*07, DRB 1*08, DRB 1*09, DRB1*10, DRB1*11, DRB 1*12, DRB 1*13, DRB 1*14, DRB 1*15, DRB1*16, DRB2*01, DRB3*01, DRB3*02, DRB3*03, DRB4*01, DRB4*02, DRB4*03, DRB5*01, DRB5*02, DRB6*01, DRB6*02, DRB7*01, DRB8*01, and DRB9*01.

In some embodiments, these alleles are matched with HLA-B alleles selected from B*07, B*08, B*13, B*14, B*15, B*18, B*27, B*35, B*37, B*38, B*39, B*40, B*41, B*42, B*44, B*45, B*46, B*47, B*48, B*49, B*50, B*51, B*52, B*53, B*54, B*55, B*56, B*57, B*58, B*59, B*67, B*73, B*78, B*81, B*82, and B*83 (or HLA-B is deleted).

In some embodiments, these alleles are matched with class II alleles are selected from DRA*01, DQA1*01, DQA1*02, DQA1*03, DQA1*04, DQA1*05, DQA1*06, DQB1*02, DQB1*03, DQB1*04, DQB1*05, DQB1*06, DPA1*01, DPA1*02, DPA1*03, DPA1*04, DPA2*01, and DPA2*02.

In some embodiments, these alleles are matched with DPB1 alleles selected from DPB1*01. DPB1*02, DPB1*03, DPB1*04, DPB1*05, DPB1*06, DPB1*08, DPB1*09, DPB1*10, DPB1*100, DPB1*101, DPB1*102, DPB1*103, DPB1*104, DPB1*105, DPB1*106, DPB1*107, DPB1*108, DPB1*109, DPB1*11, DPB1*110, DPB1*111, DPB1*112, DPB1*113, DPB1*114, DPB1*115, DPB1*116, DPB1*117, DPB1*118, DPB1*119, DPB1*120, DPB1*121, DPB1*122, DPB1*123, DPB1*124, DPB1*125, DPB1*126, DPB1*127, DPB1*128, DPB1*129, DPB1*13, DPB1*130, DPB1*131, DPB1*132, DPB1*133, DPB1*134, DPB1*135, DPB1*136, DPB1*137, DPB1*138, DPB1*139, DPB1*14, DPB1*140, DPB1*141, DPB1*142, DPB1*143, DPB1*144, DPB1*145, DPB1*146, DPB1*147, DPB1*148, DPB1*149, DPB1*15, DPB1*150, DPB1*151, DPB1*152, DPB1*153, DPB1*154, DPB1*155, DPB1*156, DPB1*157, DPB1*158, DPB1*159, DPB1*16, DPB1*160, DPB1*161, DPB1*162, DPB1*163, DPB1*164, DPB1*165, DPB1*166, DPB1*167, DPB1*168, DPB1*169, DPB1*17, DPB1*170, DPB1*171, DPB1*172, DPB1*173, DPB1*174, DPB1*175, DPB1*176, DPB1*177, DPB1*178, DPB1*179, DPB1*18, DPB1*180, DPB1*181, DPB1*182, DPB1*183, DPB1*184, DPB1*185, DPB1*186, DPB1*187, DPB1*188, DPB1*189, DPB1*19, DPB1*190, DPB1*191, DPB1*192, DPB1*193, DPB1*194, DPB1*195, DPB1*196, DPB1*197, DPB1*198, DPB1*199, DPB1*20, DPB1*200, DPB1*201, DPB1*202, DPB1*203, DPB1*204, DPB1*205, DPB1*206, DPB1*207, DPB1*208, DPB1*209, DPB1*21, DPB1*210, DPB1*211, DPB1*212, DPB1*213, DPB1*214, DPB1*215, DPB1*216, DPB1*217, DPB1*218, DPB1*219, DPB1*22, DPB1*220, DPB1*221, DPB1*222, DPB1*223, DPB1*224, DPB1*225, DPB1*226, DPB1*227, DPB1*228, DPB1*229, DPB1*23, DPB1*230, DPB1*231, DPB1*232, DPB1*233, DPB1*234, DPB1*235, DPB1*236, DPB1*237, DPB1*238, DPB1*239, DPB1*24, DPB1*240, DPB1*241, DPB1*242, DPB1*243, DPB1*244, DPB1*245, DPB1*246, DPB1*247, DPB1*248, DPB1*249, DPB1*25, DPB1*250, DPB1*251, DPB1*252, DPB1*253, DPB1*254, DPB1*255, DPB1*256, DPB1*257, DPB1*258, DPB1*259, DPB1*26, DPB1*260, DPB1*261, DPB1*262, DPB1*263, DPB1*264, DPB1*265, DPB1*266, DPB1*267, DPB1*268, DPB1*269, DPB1*27, DPB1*270, DPB1*271, DPB1*272, DPB1*273, DPB1*274, DPB1*275, DPB1*276, DPB1*277, DPB1*278, DPB1*279, DPB1*28, DPB1*280, DPB1*281, DPB1*282, DPB1*283, DPB1*284, DPB1*285, DPB1*286, DPB1*287, DPB1*288, DPB1*289, DPB1*29, DPB1*290, DPB1*291, DPB1*292, DPB1*293, DPB1*294, DPB1*295, DPB1*296, DPB1*297, DPB1*298, DPB1*299, DPB1*30, DPB1*300, DPB1*301, DPB1*302, DPB1*303, DPB1*304, DPB1*305, DPB1*306, DPB1*307, DPB1*308, DPB1*309, DPB1*31, DPB1*310, DPB1*311, DPB1*312, DPB1*313, DPB1*314, DPB1*315, DPB1*316, DPB1*317, DPB1*318, DPB1*319, DPB1*32, DPB1*320, DPB1*321, DPB1*322, DPB1*323, DPB1*324, DPB1*325, DPB1*326, DPB1*327, DPB1*328, DPB1*329, DPB1*33, DPB1*330, DPB1*331, DPB1*332, DPB1*333, DPB1*334, DPB1*335, DPB1*336, DPB1*337, DPB1*338, DPB1*339, DPB1*34, DPB1*340, DPB1*341, DPB1*342, DPB1*343, DPB1*344, DPB1*345, DPB1*346, DPB1*347, DPB1*348, DPB1*349, DPB1*35, DPB1*350, DPB1*351, DPB1*352, DPB1*353, DPB1*354, DPB1*355, DPB1*356, DPB1*357, DPB1*358, DPB1*359, DPB1*36, DPB1*360, DPB1*361, DPB1*362, DPB1*363, DPB1*364, DPB1*365, DPB1*366, DPB1*367, DPB1*368, DPB1*369, DPB1*37, DPB1*370, DPB1*371, DPB1*372, DPB1*373, DPB1*374, DPB1*375, DPB1*376, DPB1*377, DPB1*378, DPB1*379, DPB1*38, DPB1*380, DPB1*381, DPB1*382, DPB1*383, DPB1*384, DPB1*385, DPB1*386, DPB1*387, DPB1*388, DPB1*389, DPB1*39, DPB1*390, DPB1*391, DPB1*392, DPB1*393, DPB1*394, DPB1*395, DPB1*396, DPB1*397, DPB1*398, DPB1*399, DPB1*40, DPB1*400, DPB1*401, DPB1*402, DPB1*403, DPB1*404, DPB1*405, DPB1*406, DPB1*407, DPB1*408, DPB1*409, DPB1*41, DPB1*410, DPB1*411, DPB1*412, DPB1*413, DPB1*414, DPB1*415, DPB1*416, DPB1*417, DPB1*418, DPB1*419, DPB1*420, DPB1*421, DPB1*422, DPB1*423, DPB1*424, DPB1*425, DPB1*426, DPB1*427, DPB1*428, DPB1*429, DPB1*430, DPB1*431, DPB1*432, DPB1*433, DPB1*434, DPB1*435, DPB1*436, DPB1*437, DPB1*438, DPB1*439, DPB1*44, DPB1*440, DPB1*441, DPB1*442, DPB1*443, DPB1*444, DPB1*445, DPB1*446, DPB1*447, DPB1*448, DPB1*449, DPB1*45, DPB1*450, DPB1*451, DPB1*452, DPB1*453, DPB1*454, DPB1*455, DPB1*456, DPB1*457, DPB1*458, DPB1*459, DPB1*46, DPB1*460, DPB1*461, DPB1*462, DPB1*463, DPB1*464, DPB1*465, DPB1*466, DPB1*467, DPB1*468, DPB1*469, DPB1*47, DPB1*470, DPB1*471, DPB1*472, DPB1*473, DPB1*474, DPB1*475, DPB1*476, DPB1*477, DPB1*478, DPB1*479, DPB1*48, DPB1*480, DPB1*481, DPB1*482, DPB1*483, DPB1*484, DPB1*485, DPB1*486, DPB1*487, DPB1*488, DPB1*489, DPB1*49, DPB1*490, DPB1*491, DPB1*492, DPB1*493, DPB1*494, DPB1*495, DPB1*496, DPB1*497, DPB1*498, DPB1*499, DPB1*50, DPB1*500, DPB1*501, DPB1*502, DPB1*503, DPB1*504, DPB1*505, DPB1*506, DPB1*507, DPB1*508, DPB1*509, DPB1*51, DPB1*510, DPB1*511, DPB1*512, DPB1*513, DPB1*514, DPB1*515, DPB1*516, DPB1*517, DPB1*518, DPB1*519, DPB1*52, DPB1*520, DPB1*521, DPB1*522, DPB1*523, DPB1*524, DPB1*525, DPB1*526, DPB1*527, DPB1*528, DPB1*529, DPB1*53, DPB1*530, DPB1*531, DPB1*532, DPB1*533, DPB1*534, DPB1*535, DPB1*536, DPB1*537, DPB1*538, DPB1*539, DPB1*54, DPB1*540, DPB1*541, DPB1*542, DPB1*543, DPB1*544, DPB1*545, DPB1*546, DPB1*547, DPB1*548, DPB1*549, DPB1*55, DPB1*550, DPB1*551, DPB1*552, DPB1*553, DPB1*554, DPB1*555, DPB1*556, DPB1*557, DPB1*558, DPB1*559, DPB1*56, DPB1*560, DPB1*561, DPB1*562, DPB1*563, DPB1*564, DPB1*565, DPB1*566, DPB1*567, DPB1*568, DPB1*569, DPB1*57, DPB1*570, DPB1*571, DPB1*572, DPB1*573, DPB1*574, DPB1*575, DPB1*576, DPB1*577, DPB1*578, DPB1*579, DPB1*58, DPB1*580, DPB1*581, DPB1*582, DPB1*583, DPB1*584, DPB1*585, DPB1*586, DPB1*587, DPB1*588, DPB1*589, DPB1*59, DPB1*590, DPB1*591, DPB1*592, DPB1*593, DPB1*594, DPB1*595, DPB1*596, DPB1*597, DPB1*598, DPB1*599, DPB1*60, DPB1*600, DPB1*601, DPB1*602, DPB1*603, DPB1*604, DPB1*605, DPB1*606, DPB1*607, DPB1*608, DPB1*609, DPB1*61, DPB1*610, DPB1*611, DPB1*612, DPB1*613, DPB1*614, DPB1*615, DPB1*616, DPB1*617, DPB1*618, DPB1*619, DPB1*62, DPB1*620, DPB1*621, DPB1*622, DPB1*623, DPB1*624, DPB1*625, DPB1*626, DPB1*627, DPB1*628, DPB1*629, DPB1*63, DPB1*630, DPB1*631, DPB1*632, DPB1*633, DPB1*634, DPB1*635, DPB1*636, DPB1*637, DPB1*638, DPB1*639, DPB1*64, DPB1*640, DPB1*641, DPB1*642, DPB1*643, DPB1*644, DPB1*645, DPB1*646, DPB1*647, DPB1*648, DPB1*649, DPB1*65, DPB1*650, DPB1*651, DPB1*652, DPB1*653, DPB1*654, DPB1*655, DPB1*656, DPB1*657, DPB1*658, DPB1*659, DPB1*66, DPB1*660, DPB1*661, DPB1*662, DPB1*663, DPB1*664, DPB1*665, DPB1*666, DPB1*667, DPB1*668, DPB1*669, DPB1*67, DPB1*670, DPB1*671, DPB1*672, DPB1*673, DPB1*674, DPB1*675, DPB1*676, DPB1*677, DPB1*678, DPB1*679, DPB1*68, DPB1*680, DPB1*681, DPB1*682, DPB1*683, DPB1*684, DPB1*685, DPB1*686, DPB1*687, DPB1*688, DPB1*689, DPB1*69, DPB1*690, DPB1*691, DPB1*692, DPB1*693, DPB1*694, DPB1*695, DPB1*696, DPB1*697, DPB1*698, DPB1*699, DPB1*70, DPB1*700, DPB1*701, DPB1*702, DPB1*703, DPB1*704, DPB1*705, DPB1*706, DPB1*707, DPB1*708, DPB1*709, DPB1*71, DPB1*710, DPB1*711, DPB1*712, DPB1*713, DPB1*714, DPB1*715, DPB1*716, DPB1*717, DPB1*718, DPB1*719, DPB1*72, DPB1*720, DPB1*721, DPB1*722, DPB1*723, DPB1*724, DPB1*725, DPB1*726, DPB1*727, DPB1*728, DPB1*729, DPB1*73, DPB1*730, DPB1*731, DPB1*732, DPB1*733, DPB1*734, DPB1*735, DPB1*736, DPB1*737, DPB1*738, DPB1*739, DPB1*74, DPB1*740, DPB1*741, DPB1*742, DPB1*743, DPB1*744, DPB1*745, DPB1*746, DPB1*747, DPB1*748, DPB1*749, DPB1*75, DPB1*750, DPB1*751, DPB1*752, DPB1*753, DPB1*754, DPB1*755, DPB1*756, DPB1*757, DPB1*758, DPB1*759, DPB1*76, DPB1*760, DPB1*761, DPB1*762, DPB1*763, DPB1*77, DPB1*78, DPB1*79, DPB1*80, DPB1*81, DPB1*82, DPB1*83, DPB1*84, DPB1*85, DPB1*86, DPB1*87, DPB1*88, DPB1*89, DPB1*90, DPB1*91, DPB1*92, DPB1*93, DPB1*94, DPB1*95, DPB1*96, DPB1*97, DPB1*98, DPB1*99, DPB2*01, DPB2*02, and DPB2*03. In some embodiments, DPB1 is deleted.

In some embodiments, the alleles are matched with DMA*01, DMB*01, DOA*01, DOB*01, HFE*001, MICA*002, MICA*007, MICA*008, MICA*009, MICA*010, MICA*012, MICA*018, MICA*019, MICB*002, MICB*004, MICB*005, TAP1*01, TAP1*02, TAP1*03, TAP1*04, TAP1*05, TAP1*06, TAP2*01, or TAP2*02.

In some embodiments, the collection comprises cell populations or banks having at least the following HLA-C haplotypes: C*07:01 and C*04:01. It is believed that these HLC-C haplotypes are sufficient for about 46% of the US population.

In some embodiments, the collection comprises cell populations or banks having one or more of the following HLA-C haplotypes: C*07:01, C*04:01, C*07:02, C*06:02, C*03:04, and C*05:01. It is believed that these HLA-C haplotypes are sufficient for about 85% of the US population.

In some embodiments, the collection comprises cell populations or banks having one or more of the following HLA-C haplotypes: C*07:01, C*04:01, C*07:02, C*06:02, C*03:04, C*05:01, C*02:02, C*12:03, C*03:03, or C*16:01. It is believed that these HLA-C haplotypes are sufficient for about 95% of the US population.

In some embodiments, the collection comprises cell populations or banks having one or more of the following HLA-C alleles C*01:02:01:01; C*01:02:01:02; C*01:02:01:03; C*01:02:01:04; C*01:02:01:05; C*01:02:01:06; C*01:02:01:07; C*01:02:01:08; C*01:02:01:09; C*01:02:01:10; C*01:02:01:11; C*01:02:01:12; C*01:02:01:13; C*01:02:01:14; C*01:02:01:15; C*01:02:01:16; C*01:02:01:17; C*01:02:01:18; C*01:02:01:19; C*01:02:01:20; C*01:02:01:21 C*01:02:01:22; C*01:02:01:23; C*01:02:01:24; C*01:02:01:25; C*01:02:01:26; C*01:02:01:27; C*01:02:01:28; C*01:02:01:29; C*01:02:01:30; C*01:02:01:31; C*01:02:01:32; C*01:02:01:33; C*01:02:01:34; C*01:02:01:35; C*01:02:01:36; C*01:02:01:37; C*01:02:01:38; C*01:02:01:39; C*01:02:01:40; C*01:02:01:41; C*01:02:01:42; C*01:02:01:43; C*01:02:01:44; C*01:02:01:45; C*01:02:01:47; C*01:02:01:48; or C*01:02:01:50.

In some embodiments, the collection comprises cell populations or banks having one or more of the following HLA-DRB1 alleles DRB1*01:01:01:01; DRB1*01:01:01:02; DRB1*01.01.01.03; DRB1*01:01:01:04: DRB1*01:01:01:05: DRB1*01:01:02: DRB1*01.01.03; DRB1*01:01:04: DRB1*01:01:05: DRB1*01:01:06, DRB1*01:01:07: DRB1*01:01:08; DRB1*01:01:09; DRB1*01:01:10; DRB1*01:01:10; DRB1*01:01:11; DRB1*01:01:12; DRB1*01:01:13: DRB1*01:01:14; DRB1*01:01:15: DRB1*01:01:16, DRB1*01:01:17; DRB1*01:01:18; DRB1*01:01:19; DRB1*01:01:20; DRB1*01:01:21; DRB1*01.01.22; DRB1*01:01:23; DRB1*01:01:24; DRB1*01:01:25, DRB1*01:01:26; DRB1*01:01:27: DRB1*01:01:28 DRB1*01:01:29; DRB1*01:01:30: DRB1*01:01:31; DRB1*01:01:32; DRB1*01:01:33; DRB1*01:01:34; DRB1*01:01:35; DRB1*01:01:36; DRB1*01:01:37; DRB1*01:01:38: DRB1*01:01:39; DRB1*01:01:40; DRB1*01:02:01:01; DRB1*01:02:01:02; DRB1*01:02:01:03; DRB1*01:02:02; DRB1*01:02:03; or DRB1*01.02.04.

In some embodiments, the cell population in the collection retain at least one HLA-B gene, and represent at least two different HLA-B haplotypes. For example, the cell lines in the collection may represent at least four, or at least six, or at least eight, or at least ten, or at least twelve different HLA-B haplotypes. In some embodiments, the cell lines in the collection represent at least fifteen or at least twenty HLA-B haplotypes. In such embodiments, the cell lines are either homozygous for an HLA-B gene or are edited to have only a single HLA-B gene.

In some embodiments, the collection comprises cell populations or banks having at least the following HLA-B haplotypes: B*08:01 and B*07:02.

In some embodiments, the collection comprises cell populations or banks having one or more of the following HLA-B haplotypes: B*08:01, B*07:02, B*44:02, B*35:01, B*14:02, or B*40:01.

In some embodiments, the collection comprises cell populations or banks having one or more of the following HLA-B haplotypes: B*08:01, B*07:02, B*44:02, B*44:03, B*35:01, B*57:01, B*15:01, B*14:02, B*40:01, or B*53:01.

In some embodiments, the collection comprises cell populations or banks having one or more of the following HLA-B haplotypes: B*08:01, B*07:02, B*44:02, B*44:03, B*35:01, B*57:01, B*15:01, B*14:02, B*40:01, B*53:01, B*49:01, B*51:01, B*13:02, or B*38:01.

In certain embodiments, the cell populations or banks represent at least two, at least four, at least six, at least eight, or at least ten different HLA-C˜HLA-B haplotypes. In some embodiments, the cell populations in the collection represent at least twelve different HLA-C˜HLA-B haplotypes. In some embodiments, the cell populations in the collection represent at least twenty or at least twenty-five different HLA-C˜HLA-B haplotypes.

In some embodiments, the collection comprises cell populations or banks with the following haplotypes: C*07:01˜B*08:01 and C*07:02˜B*7:02. It is believed that these haplotypes are sufficient to cover about 32% of the US population.

In some embodiments, the collection comprises cell populations or banks with the following haplotypes: C*07:01˜B*08:01, C*07:02˜B*7:02, C*05:01˜B*44:02, C*16:01˜B*44:03, C*04:01˜B*35:01, C*03:04˜B*40:01, and C*08:02˜B*14:02. It is believed that these haplotypes are sufficient to cover about 61% of the US population.

In some embodiments, the collection comprises cell populations or banks with the following haplotypes: C*07:01˜B*08:01, C*07:02˜B*7:02, C*05:01˜B*44:02, C*16:01˜B*44:03, C*04:01˜B*35:01, C*03:04˜B*40:01, C*08:02˜B*14:02, C*06:02˜B*57:01, C*3:03˜B*15:01, and C*04:01˜B*53:01. It is believed that these haplotypes are sufficient to cover about 69% of the US population.

In some embodiments, the collection comprises cell populations or banks with the C*07:01˜B*08:01, C*07:02˜B*7:02, C*05:01˜B*44:02, following haplotypes: C*16:01˜B*44:03, C*04:01˜B*35:01, C*03:04˜B*40:01, C*08:02˜B*14:02, C*06:02˜B*57:01, C*3:03˜B*15:01, C*04:01˜B*53:01, C*07:01˜B*49:01, C*15:02˜B*51:01, C*06:02˜B*13:02, and C*12:03˜B*38:01. It is believed that these haplotypes are sufficient to cover about 76% of the US population.

In some embodiments, the cell populations or banks in the collection represent at least two, or at least four, or at least six, or at least eight, or at least ten different DRB1 haplotypes. In some embodiments, the cell populations in the collection represent at least twelve or at least fifteen different DRB1 haplotypes. The cell lines are either homozygous for the DRB1 gene or are edited to have only a single DRB1 gene. In various embodiments, the cell populations are also homozygous for one or more isoforms of the DR Gene, such as but not limited to, DRB2, DRB3, DRB4, and DRB5 genes, or are edited to have only a single copy of one or more of DRB2, DRB3, DRB4, and DRB5 genes. In still other embodiments, DRB2, DRB3, DRB4, and DRB5 are retained and unmodified (and may be homozygous or heterozygous across the cell lines in some embodiments).

In various embodiments, the collection comprises cell populations or banks with at least the following DRB1 haplotypes: DRB1*03:01 and DRB1*15:01.

In embodiments, the collection comprises cell populations or banks with at least the following DRB1 haplotypes: DRB1*03:01, DRB1*15:01, DRB1*04:01, DRB1*07:01, and DRB1*01:01.

In some embodiments, the collection comprises cell populations or banks with at least the following DRB1 haplotypes: DRB1*03:01, DRB1*15:01, DRB1*04:01, DRB1*07:01, DRB1*01:01, DRB1*01:02, DRB1*04:04, DRB1*13:02, DRB1*13:01, and DRB1*11:04.

In some embodiments, the collection comprises cell populations or banks with at least the following DRB1 haplotypes: DRB1*03:01, DRB1*15:01, DRB1*04:01, DRB1*07:01, DRB1*01:01, DRB1*01:02, DRB1*04:04, DRB1*13:02, DRB1*13:01, DRB1*11:04, DRB1*15:02, DRB1*03:02, and DRB1*11:01.

In some embodiments, the collection comprises cell populations or banks with at least one of the following DRB1 haplotypes: DRB1*03:01, DRB1*15:01, DRB1*04:01, DRB1*07:01, DRB1*01:01, DRB1*01:02, DRB1*04:04, DRB1*13:02, DRB1*13:01, DRB1*11:04, DRB1*15:02, DRB1*03:02, DRB1*11:01, DRB1*15:03, DRB1*04:07, DRB1*08:01, DRB1*08:03, DRB1*08:04, DRB1*08:06, DRB1*08:07, DRB1*08:11, DRB1*04:02, DRB1*14:01, DRB1*10:01.

In some embodiments, the collection comprises cell populations or banks with at least the following DRB1 haplotypes: DRB1*08:02:01 (Asian). DRB1*08:04:01 (African), DRB1*08:04:02 (North Americans), DRB1*08:04:04 (south American), and DRB1*16:01:01 (multiethnic).

In some embodiments, the cell populations or banks in the collection represent at least two, or at least four, or at least six, or at least eight, or at least ten different HLA-C˜DRB1 haplotypes. In some embodiments, the cell populations or banks in the collection represent at least twelve different HLA-C˜DRB1 haplotypes. In various embodiments, the cell populations or banks in the collection represent at least twenty or at least twenty-five different HLA-C˜DRB1 haplotypes.

In embodiments, the cell populations or banks represent at least two, or at least four, or at least six, or at least eight, or at least ten different HLA-C˜HLA-B˜DRB1 haplotypes. In some embodiments, the cell populations or banks in the collection represent at least twelve different HLA-C˜HLA-B˜DRB1 haplotypes. In various embodiments, the cell populations in the collection represent at least twenty or at least twenty-five different HLA-C˜HLA-B˜DRB1 haplotypes.

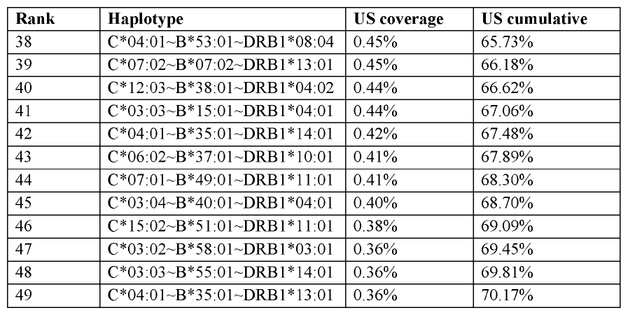

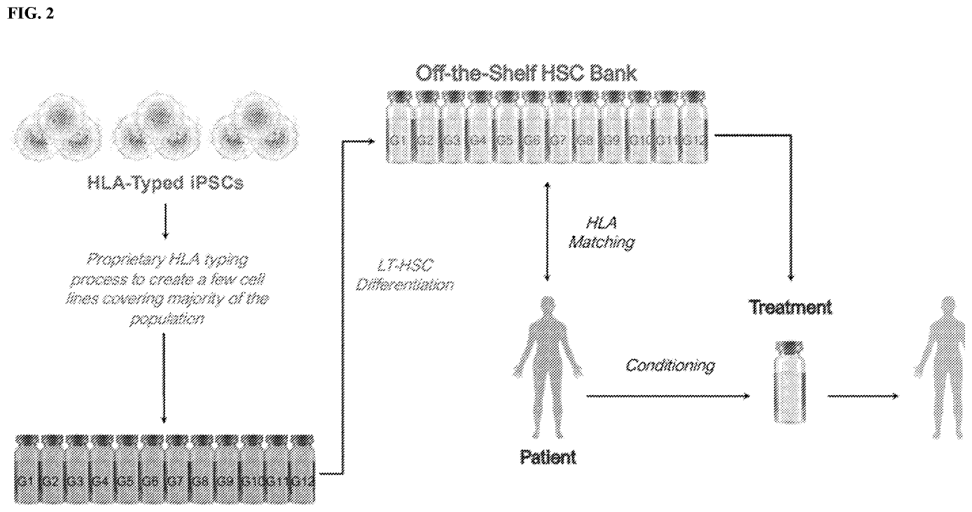

In various embodiments, the collection comprises cell populations or banks with at least the following haplotypes: C*07:01˜B*08:01˜DRB1*03:01 and C*07:02˜B*07:02˜DRB1*15:01. It is believed that these haplotypes are sufficient to cover about 22% of the US population. See FIG. 2.

In some embodiments, the collection comprises cell populations or banks with at least the following haplotypes: C*07:01˜B*08:01˜DRB1*03:01, C*07:02˜B*07:02˜DRB1*15:01, C*05:01˜B*44:02˜DRB1*04:01, C*16:01˜B*44:03˜DRB1*07:01, C*04:01˜B*35:01˜DRB1*01:01, C*06:02˜B*57:01˜DRB1*07:01, C*06:02˜B*13:02˜DRB1*07:01, and C*08:02˜B*14:02˜DRB1*01:02. It is believed that these haplotypes are sufficient to cover about 40% of the US population.

In some embodiments, the collection comprises cell populations or banks with at least the following haplotypes: C*07:01˜B*08:01˜DRB1*03:01, C*07:02˜B*07:02˜DRB1*15:01, C*05:01˜B*44:02˜DRB1*04:01, C*16:01˜B*44:03˜DRB1*07:01, C*04:01˜B*35:01˜DRB1*01:01, C*06:02˜B*57:01˜DRB1*07:01, C*06:02˜B*13:02˜DRB1*07:01, C*08:02˜B*14:02˜DRB1*01:02, C*3:04˜B*40:01˜DRB1*04:04, C*04:01˜B*44:03˜DRB1*07:01, C*03:04˜B*40:01˜DRB1*13:02, C*03:04˜B*15:01˜DRB1*04:01, C*05:01˜B*18:01˜DRB1*03:01, C*05:01˜B*44:02˜DRB1*13:01, C*07:02˜B*07:02˜DRB1*01:01, and C*04:01˜B*35:02˜DRB1*11:04. It is believed that these haplotypes are sufficient to cover about 51% of the US population.

In some embodiments, the collection comprises cell populations or banks with at least the following haplotypes: C*07:01˜B*08:01˜DRB1*03:01, C*07:02˜B*07:02˜DRB1*15:01, C*05:01˜B*44:02˜DRB1*04:01, C*16:01˜B*44:03˜DRB1*07:01, C*04:01˜B*35:01˜DRB1*01:01, C*06:02˜B*57:01˜DRB1*07:01, C*06:02˜B*13:02˜DRB1*07:01, C*08:02˜B*14:02˜DRB1*01:02, C*3:04˜B*40:01˜DRB1*04:04, C*04:01˜B*44:03˜DRB1*07:01, C*03:04˜B*40:01˜DRB1*13:02, C*03:04˜B*15:01˜DRB1*04:01, C*05:01˜B*18:01˜DRB1*03:01, C*05:01˜B*44:02˜DRB1*13:01, C*07:02˜B*07:02˜DRB1*01:01, C*04:01˜B*35:02˜DRB1*11:04, C*12:02˜B52:01˜DRB1*15:02, C*03:03˜B*15:01˜DRB1*13:01, C*07:02˜B*07:02˜DRB1*07:01, C*05:01˜B*44:02˜DRB1*15:01, C*12:03˜B*38:01˜DRB1*13:01, C*17:01˜B*42:01˜DRB1*03:02, C*08:02˜B*14:01˜DRB1*07:01, C*01:02˜B*27:05˜DRB1*01:01, C*04:01˜B*35:01˜DRB1*11:01, C*06:02˜B*50:01˜DRB1*07:01, C*07:01˜B*18:01˜DRB1*11:04, and C*04:01˜B*53:01˜DRB1˜*13:02. It is believed that these haplotypes are sufficient to cover about 60% of the US population.

In some embodiments, the collection comprises cell populations or banks with at least the following haplotypes: C*07:01˜B*08:01˜DRB1*03:01, C*07:02˜B*07:02˜DRB1*15:01, C*05:01˜B*44:02˜DRB1*04:01, C*16:01˜B*44:03˜DRB1*07:01, C*04:01˜B*35:01˜DRB1*01:01, C*06:02˜B*57:01˜DRB1*07:01, C*06:02˜B*13:02˜DRB1*07:01, C*08:02˜B*14:02˜DRB1*01:02, C*3:04˜B*40:01˜DRB1*04:04, C*04:01˜B*44:03˜DRB1*07:01, C*03:04˜B*40:01˜DRB1*13:02, C*03:04˜B*15:01˜DRB1*04:01, C*05:01˜B*18:01˜DRB1*03:01, C*05:01˜B*44:02˜DRB1*13:01, C*07:02˜B*07:02˜DRB1*01:01, C*04:01˜B*35:02˜DRB1*11:04, C*12:02˜B52:01˜DRB1*15:02, C*03:03˜B*15:01˜DRB1*13:01, C*07:02˜B*07:02˜DRB1*07:01, C*05:01˜B*44:02˜DRB1*15:01, C*12:03˜B*38:01˜DRB1*13:01, C*17:01˜B*42:01˜DRB1*03:02, C*08:02˜B*14:01˜DRB1*07:01, C*01:02˜B*27:05˜DRB1*01:01, C*04:01˜B*35:01˜DRB1*11:01, C*06:02˜B*50:01˜DRB1*07:01, C*07:02˜B*07:02˜DRB1*11:01, C*04:01˜B*53:01˜DRB1˜*13:02, C*12:03˜B*18:01˜DRB1*15:01, C*07:02˜B*07:02˜DRB1*11:01, C*05:01˜B*44:02˜DRB1*01:01, C*04:01˜B*53:01˜DRB1*15:03, C*07:02˜B*07:02˜DRB1*04:01, C*08:02˜B*14:02˜DRB1*13:02, C*04:01˜B*35:01˜DRB1*07:01, C*07:01˜B*08:01˜DRB1*15:01, C*07:02˜B*39:05˜DRB1*04:07, C*04:01˜B*53:01˜DRB1*08:04, C*07:02˜B*07:02˜DRB1*13:01, C*12:03˜B*38:01˜DRB1*04:02, C*03:03˜B*15:01˜DRB1*04:01, C*04:01˜B*35:01˜DRB1*14:01, C*06:02˜B*37:01˜DRB1*10:01, C*07:01˜B*49:01˜DRB1*11:01, C*03:04˜B*40:01˜DRB1*04:01, C*15:02˜B*51:01˜DRB1*11:01, C*03:02˜B*58:01˜DRB1*03:01, C*03:03˜B*55:01˜DRB1*14:01, and C*04:01˜B*35:01˜DRB1*13:01. It is believed that these haplotypes are sufficient to cover about 70% of the US population.

In various embodiments, the cell populations or banks retain HLA-E, HLA-F, and HLA-G genes, which can be homozygous or heterozygous across the collection (e.g., are unmodified).

In some embodiments, the cell populations or banks have a deletion, inactivation, or mutagenesis of one or more HLA-DP genes, which may be selected from DPA1, DPA2, DPA3, DPB1, and DPB2. In some embodiments, the cell populations or banks have a deletion or inactivation of one or both HLA-DPA1 genes and/or one or both HLA-DPB1 genes. In some embodiments, the cell populations or banks are homozygous for or retain at least one HLA-DPA1 gene and at least one HLA-DPB1 gene. In some embodiments, the cell lines or banks retain DPA2, DPA3, and DPB2 (and which are unmodified, and may be homozygous or heterozygous across the cell populations or banks).

In some embodiments, the cell populations or banks have a deletion, inactivation, or mutagenesis of one or more HLA-DQ genes, which can be selected from DQA1, DQA2, DQB1, DQB2, and DQB3. In some embodiments, one or both HLA-DQA1 genes are deleted. In these or other embodiments, one or both HLA-DQB1 genes are deleted. In some embodiments, DQA2, DQB2, and DQB3 are retained.

In some embodiments, the cell populations or banks have a deletion or inactivation of both HLA-DQA1 and both HLA-DQB1 genes. Alternatively, the cell populations or banks are homozygous for or retain at least one copy of HLA-DQA1 and HLA-DQB1 genes. In some embodiments, the cell populations or banks are unmodified at HLA-DQA1 and HLA-DQB1 loci (and may be homozygous and heterozygous across the cell lines).

In exemplary embodiments, the cell populations or banks have both HLA-A genes deleted, and both DPB1 genes and/or both DQB1 genes are deleted. In such embodiments, the cell lines are homozygous for or retain only single copies of HLA-B, HLA-C, HLA-DRB1. Both copies of all other HLA genes (particularly those not annotated as pseudogenes) are retained, and these genes may be homozygous or heterozygous. In some embodiments, both copies of HLA-B are deleted.

In various embodiments, the cell populations are induced pluripotent stem cell (iPSC) lines. In some embodiments, the cell populations are hematopoietic stem cell (HSC) lines. For example, HSC populations may be prepared from iPSCs (having the desired gene deletions or inactivations) by a method described herein. In various embodiments, the iPSC population is a human iPSC population derived from lymphocytes, cord blood cells, peripheral blood mononuclear cells, CD34+ cells, or human primary tissues, as described herein.

In some embodiments, cells are derived from the HSCs for administration to a recipient, and the cells may be any of the hematopoietic lineages. For example, the hematopoietic lineage may be selected from common lymphoid precursor (CLP) cells, granulocyte-monocyte progenitor (GMP) cells, progenitor-T cells, T lymphocytes, B lymphocytes, Natural Killer cells, neutrophils, monocyte, macrophages, dendritic cells, red cells, megakaryocytes, and platelets. T cells may be CD4+ helper T cells, CD8+ cytotoxic T cells, or regulatory T cells (Tregs).

In some embodiments, cells, including but not limited to HSCs, hematopoietic progenitors, and/or hematopoietic lineages (as described above), are derived from a allogeneic donor, patient, or banked tissues or cells.

In some embodiments, the cells are non-hematopoietic stem cells or precursor cells, or cells differentiated therefrom. Exemplary stem cells include mesenchymal stem cells, neural stem cells, and epithelial stem cells. In various embodiments, iPSC lines are used to produce various non-hematopoietic cells and tissues, including those selected from neurons (including cortical, dopaminergic, and motor neurons), astrocytes, oligodendrocytes, cardiomyocytes, cornea, chondrons, skeletal muscle cells, hepatocytes, pancreatic β cells, and lung epithelial cells. Protocols for deriving such cells and tissues are known in the art.

In various embodiments, the cell populations in the bank are each contained within separate containers suitable for maintaining viability of the cell lines or cell compositions for expansion, differentiation, or administrations. The cell composition of this disclosure may further comprise a pharmaceutically acceptable carrier or vehicle suitable for intravenous infusion or other administration route, and the composition may include a suitable cryoprotectant. An exemplary, on-limiting, carrier is DMSO (e.g., about 10% DMSO).

In other aspects, this disclosure provides a method for cell therapy. The method comprises administering to a recipient in need thereof a cell population derived from a cell population within the collection or bank of this disclosure. In various embodiments, the administered cell population or tissue is matched with the recipient for HLA-C. In embodiments where the administered cell population or tissue retains at least one HLA-B gene, the cell population or tissue is further matched with the recipient for HLA-B. In some embodiments, where the administered cell population or tissue retains at least one DRB1 gene, the cell population or tissue is further matched with the recipient for DRB1. In some embodiments, where the administered cell population or tissue retains at least one HLA-C and DRB1 genes, the cell population or tissue is further matched with the recipient for HLA-C and DRB1. In some embodiments, where the administered cell population or tissue retains at least one DQB1 gene, the cell population or tissue is further matched with the recipient for DQB1. In still other embodiments, the cell population is not matched to the recipient for DQB1. In various embodiments, all other loci are unmatched.

In some embodiments, a method for treating a subject according to the present disclosure comprises: (a) expanding a population of pluripotent stem cells (e.g., iPSCs) according to the methods described herein; (b) preparing HSCs or progenies thereof (as described herein); and (c) introducing the population of hematopoietic stem cells or progenies thereof into the subject. Optionally, the hematopoietic stem cells may be differentiated into common megakaryocyte-erythroid progenitor cells, lymphoid progenitor cells, progenitor T and/or B cells, common myeloid progenitor cells, granulocytes, granulocyte-megakaryocyte progenitor cells, promyelocytes, basophils, eosinophils, neutrophils, erythrocytes, reticulocytes, thrombocytes, megakaryoblasts, platelet-producing megakaryocytes, platelets, monocytes, macrophages, dendritic cells, microglia, osteoclasts, lymphocytes, NK cells, B-cells and/or T-cells prior to their administration.

In some embodiments a cell composition is provided comprising: (a) a culture medium optionally comprising cytokines and/or growth factors; and (b) one or more hematopoietic lineage cells or populations thereof derived from the cells of the present disclosure, wherein the hematopoietic lineage cells are: (i) derivatives of cell line(s) or banks of expanded primary cells (e.g., iPSCs), such as endothelial or hemogenic endothelium (HE), wherein the endothelial or HE cells are CD34+ and at least identified as CD43− or CD93− or CXCR4− or CD73− or CXCR4−CD73− or CD34+CD45+; CD34+CD31+, CD34+CD144+; (ii) hematopoietic stem cells identified as CD34+CD90+CD38−; (iii) pluripotent stem cell-derived T cell progenitors, wherein the T cell progenitors are CD34+CD45+CD7+ or CD34−CD45+CD7+ or CD34−CD7+CD5−CD1a−, optionally including thymus engrafting receptors like CCR7; (iv) pluripotent stem cell-derived T cells, wherein the T cells are CD45+CD3+CD4+ or CD45+CD3+CD8+; (v) pluripotent stem cell-derived NK cell progenitors, wherein the NK cell progenitors are CD45+CD56+CD7+; (vi) pluripotent stem cell-derived NK cells, wherein the NK cells are CD3−CD45+CD56+, and optionally further defined by NKp46+, CD57+, and CD16+, (vii) pluripotent stem cell-derived NKT cells, wherein the NKT cells are CD45+ Valpha 24J alpha 18+CD3+; (viii) pluripotent stem cell-derived B cells, wherein the B cells are CD45+CD19+; (ix) pluripotent stem cell-derived megakaryocytes, wherein the megakaryocytes are CD41 and/or CXCR4, optionally they are CXCR4+ or Glycoprotein V/CD42d+ or CXCR1/IL-8RA+ or CXCR2/IL-8RB+I or integrin alpha 2b/CD41+ or LIFR alpha or Thrombopoietin R/Tpo R+; (x) pluripotent stem cell-derived platelets, wherein the platelets are CD41+ (GP IIb/IIIa), or CD42a+ (GPIX), or CD42b+ (GPIb), or CD61+ (xi); pluripotent stem cell derived erythrocytes, wherein the erythrocytes are CD235a+; (xii) pluripotent stem cell derived neutrophils, wherein the neutrophils are CD15+, CD16+, CD49d(−), and (xiii) pluripotent stem cell derived granulocyte monocyte progenitors (GMPs).

In some embodiments, the cell populations are used to generate cell therapies to treat human diseases including but not limited to, a hematological malignancy, aplastic anemia, hemoglobinopathy, inborn error of metabolism, and severe immunodeficiency. For example, the subject may have a condition selected from acute myeloid leukemia; acute lymphoblastic leukemia; chronic myeloid leukemia; chronic lymphocytic leukemia; acute lymphatic leukemia, aplastic anemia, Krabbe Disease, bone marrow failure syndromes, Hurler Syndrome, Leukodystrophies, Myelodysplastic syndromes, POEMS syndrome, Primary amyloidosis, myeloproliferative disorder; myelodysplastic syndrome; multiple myeloma; Non-Hodgkin lymphoma; Hodgkin disease; aplastic anemia; pure red-cell aplasia; paroxysmal nocturnal hemoglobinuria; Fanconi anemia; thalassemia major; sickle cell anemia; severe combined immunodeficiency (SCID); acquired immune deficiency syndrome (AIDS); Wiskott-Aldrich syndrome; hemophagocytic lymphohistiocytosis; inborn errors of metabolism; epidermolysis bullosa; severe congenital neutropenia; Shwachman-Diamond syndrome; Diamond-Blackfan anemia; eukocyte adhesion deficiency; X-linked forms of SCID, Sickle cell anemia, Alpha thalassemia, Beta thalassemia, Delta thalassemia, Hemoglobin E/thalassemia, Hemoglobin S/thalassemia, Hemoglobin C/thalassemia, Hemoglobin D/thalassemia, Chronic granulomatous disease, X-linked Chronic granulomatous disease, autosomal recessive (AR) chronic granulomatous disease, chronic granulomatous disease AR I NCF1, Chronic granulomatous disease AR CYBA, Chronic granulomatous disease AR II NCF2, Chronic granulomatous disease AR III NCF4, X-linked Severe Combined Immune Deficiency (SCID), IL7-RA SCID, CD3 SCID, Rag1/Rag2 SCID, ADA SCID, Artemis SCID, CD45 SCID, Jak3 SCID, Congenital agranulocytosis, Congenital agranulocytosis-congenital neutropenia-SCN1, Congenital agranulocytosis-congenital neutropenia-SCN2, Familial hemophagocytic lymphohistiocystosis (FHL), Familial hemophagocytic lymphohistiocytosis type 2 (FHL2, perforin mutation), Agammaglobulinemia (X-linked Agammaglobulinemia), Wiskott-Aldrich syndrome, Chediak-Higashi syndrome, Hemolytic anemia due to red cell pyruvate kinase deficiency, Paroxysmal nocturnal hemoglobinuria, X-linked Adrenoleukodystrophy (X-ALD), X-linked lymphoproliferative disease, Acquired idiopathic sideroblastic anemia, Systemic mastocytosis, Von willebrand disease (VWD), Congenital dyserythropoietic anemia type 2, Cartilage-hair hypoplasia syndrome, Unicentric Castleman's Disease, Multicentric Castleman's Disease, Congenital amegakaryocytic thrombocytopenia (CAMT) type I, Reticular dysgenesis, Hereditary spherocytosis, Blackfan-Diamond syndrome, Shwachman-Diamond syndrome, Mucopolysaccharidoses, Lesch-Nyhan syndrome, Glycogen storage disease, Congenital mastocytosis, Omenn syndrome, X-linked Immunodysregulation, polyendocrinopathy, Thrombocytopenia-absent radius syndrome, Osteopetrosis, Infantile osteopetrosis, and enteropathy (IPEX), IPEX characterized by mutations in FOXP3, X-linked syndrome of polyendocrinopathy, immune dysfunction, and diarrhea (XPID), X-Linked Autoimmunity-Allergic Dysregulation Syndrome (XLAAD), IPEX-like syndrome, Hyper IgM type 1, Hyper IgM type 2, Hyper IgM type 3, Hyper IgM type 4, Hyper IgM type 5, X linked hyperimmunoglobulin M, Bare lymphocyte Syndrome type I, or Bare lymphocyte Syndrome type II, myasthenia gravis, rheumatoid arthritis, multiple sclerosis, type I diabetes mellitus, idiopathic inflammatory myopathy, systemic lupus crythematosus (SLE), myasthenia gravis, Grave's disease, dermatomyositis, polymyositis, Crohn's disease, ulcerative colitis, gastritis, Hashimoto's thyroiditis, asthma, psoriasis, psoriatic arthritis, dermatitis, systemic scleroderma and sclerosis, inflammatory bowel disease (IBD), respiratory distress syndrome, meningitis, encephalitis, uveitis, glomerulonephritis, eczema, atherosclerosis, leukocyte adhesion deficiency, Raynaud's syndrome, Sjorgen's syndrome, Reiter's disease, Beheet's disease, immune complex nephritis, IgA nephropathy, IgM polyneuropathies, immune-mediated thrombocytopenias e.g., ITP), acute idiopathic thrombocytopenia purpura, chronic idiopathic thrombocytopenia purpura, hemolytic anemia, lupus nephritis, atopic dermatitis, pemphigus vulgaris, opsoclonus-myoclonus syndrome, pure red cell aplasia, mixed cryoglobulinemia, ankylosing spondylitis, hepatitis C-associated cryoglobulinemic vasculitis, chronic focal encephalitis, bullous pemphigoid, hemophilia A, membranoproliferative glomerulonephritis, adult and juvenile dermatomyositis, adult polymyositis, chronic urticaria, primary biliary cirrhosis, neuromyelitis optica, Graves' dysthyroid disease, bullous pemphigoid, membranoproliferative glomerulonephritis, Churg-Strauss syndrome, juvenile onset diabetes, hemolytic anemia, atopic dermatitis, systemic sclerosis, Sjorgen's syndrome and glomerulonephritis, dermatomyositis, ANCA, aplastic anemia, autoimmune hemolytic anemia (AIHA), factor VIII deficiency, hemophilia A, autoimmune neutropenia, Castleman's syndrome, Goodpasture's syndrome, solid organ transplant rejection, graft versus host disease (GVHD), autoimmune hepatitis, lymphoid interstitial pneumonitis, HIV, bronchiolitis obliterans (non-transplant), Guillain-Barre Syndrome, large vessel vasculitis, giant cell (Takayasu's) arteritis, medium vessel vasculitis, Kawasaki's Disease, polyarteritis nodosa, Wegener's granulomatosis, microscopic polyangiitis (MPA), Omenn's syndrome, Alzheimer, chronic renal failure, acute infectious mononucleosis, or HIV and herpes virus associated diseases.

In some embodiments, the cell populations are used to generate cell therapies to treat cancer, including but not limited to, solid and liquid cancers, acquired diseases, congenital diseases, and non-hematopoietic diseases, including but not limited to diseases affecting neurons, astrocytes, oligodendrocytes, cardiomyocytes, skeletal muscle cells, hepatocytes, pancreatic β cells, lung epithelial cells, etc.

In some embodiments, an immune cell lineage (derived from cell populations or banks described herein) is administered to a patient in need thereof. For example, the immune cell lineage may be a T cell, NK cell, B-cell, or macrophage. In some embodiments, the T cell lineage is a T-regulatory cell or cytotoxic T cell. In some embodiments, the T cell expresses a heterologous TCR or a chimeric antigen receptor (CAR). In various embodiments, the recipient (patient) has a condition selected from one or more of lymphopenia, cancer, immune deficiency, autoimmune disease, skeletal dysplasia, a bone marrow failure syndrome, genetic disorder impacting the immune system, cardiac failure, neural disorders, immunodeficiency, blood disorders (e.g., Thalassemia, Anemias, sickle cell disease), heart disease, liver disease, multiple sclerosis, muscular dystrophy, skin and tissue regeneration, spinal cord degeneration, trauma, stroke, neurodegenerative diseases (e.g., Alzheimer, dementia, down's syndrome or Parkinson), metabolic disorder, hematopoietic stem cell transplant (HPSCT), i.e., administration of healthy hematopoietic stem cells to patients with dysfunctional or depleted bone marrow, thrombocytopenia, or cancer.

In various embodiments, the recipient has undergone lympho-deleting therapy, cyto-reductive therapy, or immunomodulatory therapy prior to administration of the cell therapy. In some embodiments, derivatives of the cell line(s) or banks of expanded primary cells (e.g., HSCs and/or progenies derived therefrom) disclosed herein are administered to reconstitute the recipient's hematopoietic system. Cell lineages generated using the methods described herein are administered to the subject e.g., by intravenous infusion. In some embodiments, the methods can be performed following myeloablative, non-myeloablative, or immunotoxin-based (e.g., anti-c-Kit, anti-CD45, etc.) conditioning regimes.

In still other embodiments, a cell or tissue derived from the cell population is administered to a recipient in need thereof. Exemplary cells include mesenchymal stem cells, neural stem cells, corneal epithelium/endothelium and RPE, epithelial stem cells, neuronal cells (or precursors thereof) (including cortical, dopaminergic, and motor neurons, or precursors thereof), astrocytes (or precursors thereof), oligodendrocytes (or precursors thereof), cardiomyocytes (or precursors thereof), skeletal muscle cells (or precursors thereof), hepatocytes (or precursors thereof), pancreatic β cells (or precursors thereof), and lung epithelial cells (or precursors thereof). Such cells can be administered to treat or ameliorate any disease or condition (including genetic or acquired condition) afflicting the relevant tissue or organ. Such tissues or organs include but are not limited to the central nervous system, skeletal muscle, heart, liver, pancreas, or lung.

In other aspects, this disclosure provides a method for making a cell population of the disclosure. The method can comprise providing an iPSC population that is HLA-modified according to this disclosure; enriching for CD34+ cells from a differentiated iPSC population (e.g., embryoid bodies, or EBs) to prepare a CD34+-enriched population; and inducing endothelial-to-hematopoietic transition of the CD34+-enriched population (e.g., for at least two days but no more than 12 days), to prepare a population comprising hematopoietic stem cells (HSCs) and/or hematopoietic stem progenitor cells (HSPCs). In some embodiments, the method further comprises harvesting a CD34+-enriched population that are undergoing endothelial-to-hematopoietic transition. For example, this can include harvesting of CD34+ floater and/or adherent cells, but generally will comprise at least non-adherent cells.

Conventionally, hematopoietic lineages are prepared by differentiation of iPSCs to embryoid bodies up to day 8 to harvest CD34+ cells. CD34 is commonly used as a marker of hemogenic endothelial cells, hematopoietic stem cells, and hematopoietic progenitor cells. In accordance with embodiments of this disclosure, endothelial-to-hematopoietic transition (EHT) is induced in a CD34+ cell population, and which can be derived from iPSC-embryoid bodies, and optionally used for the ex vivo generation of hematopoietic lineages.

In various embodiments, iPSCs are prepared by reprogramming somatic cells. The term “induced pluripotent stem cell” or “iPSC” refers to cells derived from somatic cells, such as skin, bone marrow, umbilical cord blood or peripheral blood cells that have been reprogrammed back into an embryonic-like pluripotent state. In some embodiments, iPSCs are generated from somatic cells such as (but not limited to) fibroblasts or PBMCs (or cells isolated therefrom). In some embodiments, the iPSCs are derived from lymphocytes (e.g., T-cells, B-cells, NK-cells, etc.), cord blood cells (including from CD3+ or CD8+ cells from cord blood), PBMCs, CD34+ cells, or other human primary tissues. In some embodiments, iPSCs are derived from CD34+ cells isolated from peripheral blood.

Somatic cells may be reprogrammed by expression of reprogramming factors selected from Sox2, Oct3/4, c-Myc, Nanog, Lin28, and klf4. In some embodiments, the reprogramming factors are Sox2, Oct3/4, c-Myc, Nanog, Lin28, and klf4. In some embodiments, the reprogramming factors are Sox2, Oct3/4, c-Myc, and klf4. Methods for preparing iPSCs are described, for example, in U.S. Pat. Nos. 10,676,165; 9,580,689; and 9,376,664, which are hereby incorporated by reference in their entireties. In various embodiments, reprogramming factors are expressed using well known viral vector systems, such as lentiviral, Sendai, or measles viral systems. Alternatively, reprogramming factors can be expressed by introducing mRNA(s) encoding the reprogramming factors into the somatic cells. Further still, iPSCs may be created by introducing a non-integrating episomal plasmid expressing the reprogramming factors, i.e., for the creation of transgene-free and virus-free iPSCs. Known episomal plasmids can be employed with limited replication capabilities and which are therefore lost over several cell generations. Alternative methods include minicircle vectors, PiggyBac transposons, and exosome incorporation.

In some embodiments, the iPSC population is gene edited to delete or inactivate one or more HLA genes. The selection of HLA genes for deletion or inactivation is as already described. The deletion or inactivation refers to a genetic modification of the target gene (i.e., gene edit) that abrogates functional expression of the corresponding gene product (i.e., the corresponding polypeptide). Such gene edits include full or partial gene deletions, as well as deletions of critical cis-acting expression control sequences. For example, with respect to HLA class I genes, deletions can include deletions of one or more extracellular domains such as α1, α2, and α3 domains. In some embodiments, HLA class I deletions include deletions of the transmembrane domain. With respect to HLA class II genes, deletions can include deletions of one or more extracellular domains such as α1 and/or α2, or B1 and/or B2. In some embodiments, HLA class II deletions include deletions of the transmembrane domain. In some embodiments, the HLA deletions comprise deletions of the entire coding sequence or substantially the entire coding sequence. In some embodiments, deletions are targeted toward exon 1 and/or exon 2 of HLA genes, and includes in various embodiments a deletion of at least 50 base pairs, at least 100 base pairs, at least 250 base pairs, or at least 500 base pairs. In still other embodiments, gene deletions or inactivations alter critical expression control sequences such as promoters, cis-acting sequences bound by transcriptional activators, or ribosomal binding sequences, to thereby substantially reduce or eliminate expression.

In various embodiments, a sgRNA targeting HLA-A can target a region of chromosome 6 defined as 29942532-29942626. In various embodiments, a sgRNA targeting HLA-DQB1 can target a region of chromosome 6 defined as 32665067-32664798. In various embodiments, a sgRNA targeting HLA-DPB1 can target a region of chromosome 6 defined as 33080672-33080935. See FIG. 11B.

The target HLA loci is deleted or inactivated using one or more gene modifying tools such as CRISPR-Cas (e.g., CRISPR-Cas9, CRISPR-Cas12, STAR-CRISPR, CRISPR-CasX, CRISPR-associated transposases), RNA-editors, insulated genomic domain-platform editing, and combinations thereof. In some embodiments, the target HLA loci can also be deleted or inactivated using siRNAs, oligonucleotides, and/or zinc finger nucleases. In some embodiments, the HLA modifications are conducted by CRISPR-Cas9, and which may employ one or a combination of gRNAs (e.g., sgRNAs) comprising a spacer sequence listed in Tables 2A-17 for the particular HLA gene and haplotype.

Generally, various editing technologies are known, which can be applied according to various embodiments of this disclosure. Gene editing technologies include but are not limited to zinc fingers (ZFs), and transcription activator-like effectors (TALEs), etc. Fusion proteins containing one or more of these DNA-binding domains and the cleavage domain of Fokl endonuclease can be used to create a double-strand break in a desired region of DNA in a cell (See, e.g., US Patent Appl. Pub. No. US 2012/0064620, US Patent Appl. Pub. No. US 2011/0239315, U.S. Pat. No. 8,470,973, US Patent Appl. Pub. No. US 2013/0217119, U.S. Pat. No. 8,420,782, US Patent Appl. Pub. No. US 2011/0301073, US Patent Appl. Pub. No. US 2011/0145940, U.S. Pat. Nos. 8,450,471, 8,440,431, 8,440,432, and US Patent Appl. Pub. No. 2013/0122581, the contents of all of which are hereby incorporated by reference). In some embodiments, gene editing is conducting using CRISPR associated Cas system (e.g., CRISPR-Cas9), as known in the art. See, for example, U.S. Pat. Nos. 8,697,359, 8,906,616, and 8,999,641, each of which is hereby incorporated by reference in its entirety. In various embodiments, the gene editing employs a Type II Cas endonuclease (such as Cas9) or employs a Type V Cas endonuclease (such as Cas12a). Type II and Type V Cas endonucleases are guide RNA directed. Design of gRNAs to guide the desired gene edit (while limiting or avoiding off target edits) is known in the art. See, for example, Mohr S E, et al., CRISPR guide RNA design for research applications, FEBS J. 2016 September; 283 (17): 3232-3238. In still other embodiments, non-canonical Type II or Type V Cas endonucleases having homology (albeit low primary sequence homology) to S. pyogenes Cas9 or Prevotella and Francisella1 (Cpf1 or Cas12a) can be employed. Numerous such non-canonical Cas endonucleases are known in the art. Nidhi S, et al. Novel CRISPR-Cas Systems: An Updated Review of the Current Achievements, Applications, and Future Research Perspectives, Int J Mol Sci. 2021 April; 22 (7): 3327. In still other embodiments, the gene editing employs base editing or prime editing to incorporate mutations without instituting double strand breaks. See, for example, Antoniou P, et al., Base and Prime Editing Technologies for Blood Disorders, Front. Genome Ed., 28 Jan. 2021; Matsuokas IG, Prime Editing: Genome Editing for Rare Genetic Diseases Without Double-Strand Breaks or Donor DNA, Front. Genet., 9 Jun. 2020. Various other gene editing processes are known, including use of dead Cas (dCas) systems (e.g., Cas fusion proteins) to target DNA modifying enzymes to desired targets using the dCas as a guide RNA-directed system. Brezgin S, Dead Cas Systems: Types, Principles, and Applications, Int J Mol Sci. 2019 December; 20 (23): 6041.

Base editors that can install precise genomic alterations without creating double-strand DNA breaks can also be used in gene editing (e.g., designing gene therapy vectors) in the cells (e.g., iPSCs). Base editors essentially comprise a catalytically disabled nuclease, such as Cas9 nickase (nCas9), which is incapable of making DSBs and is fused to a nucleobase deaminase enzyme and, in some cases, a DNA glycosylase inhibitor. Currently, there are 2 major categories of base editors, cytidine base editors (CBEs) and adenine base editors (ABEs), which catalyze C>T and A>G transitions. Base editors can be delivered, for example, via HDAd5/35++ vectors to efficiently edit promoters and enhancer to active or inactivate a gene. Exemplary methods are described in U.S. Pat. Nos. 9,840,699; 10,167,457; 10,113,163; 11,306,324; 11,268,082; 11,319,532; and 11,155,803. Also contemplated are prime editors that comprise a reverse transcriptase conjugated to (e.g., fused with) a Cas endonuclease and a polynucleotide useful as a DNA synthesis template conjugated to (e.g., fused with) a guide RNA, as described in WO2020191153A2.

Exemplary vectors that can be used for the genome editing applications include, but are not limited to, plasmids, retroviral vectors, lentiviral vectors, adenovirus vectors (e.g., Ad5/35, Ad5, Ad26, Ad34, Ad35, Ad48, parvovirus (e.g., adeno-associated virus (AAV) vectors, herpes simplex virus vectors, baculoviral vectors, coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including herpes virus (e.g., Herpes Simplex virus types 1 and 2, Epstein-Barr virus, cytomegalovirus), and poxvirus (e.g., canarypox, vaccinia or modified vaccinia virus. The vector comprising the nucleic acid molecule of interest may be delivered to the cell (e.g., iPS cells, endothelial cells, hemogenic endothelial cells, HSCs (ST-HSCs or LT-HSCs) via any method known in the art, including but not limited to transduction, transfection, infection, and electroporation. Any of these vectors may include transposable element (such as a piggyback transposon or sleeping beauty transposon). Transposons insert specific sequences of DNA into genomes of vertebrate animals. The gene of interest can be integrated into the genome of a mammalian cell by transposase-catalyzed cleavage of similar excision sites that exist within the nuclear genome of the cell.

For increased efficiency, in some embodiments, the Cas and the gRNA are combined before being delivered into cells. The Cas-gRNA complex is known as a ribonucleoprotein (RNP). A number of methods have been developed for direct delivery of RNPs to cells. For example, RNP can be delivered into cells in culture by lipofection or electroporation. Electroporation using a nucleofection protocol is often preferred, as this allows the RNP to enter the nucleus of cells quickly, so it can immediately start cutting the genome. See, for example, Zhang S, Shen J, Li D, Cheng Y. Strategies in the delivery of Cas9 ribonucleoprotein for CRISPR/Cas9 genome editing. Theranostics. 2021 Jan. 1; 11 (2): 614-648, hereby incorporated by reference in its entirety. In some embodiments, Cas9 and gRNA are electroporated as RNP into the donor PBMC-derived iPSCs and/or HSCs.

Generally, a protospacer adjacent motif (PAM) is required for a Cas nuclease to cut and is generally found 3-4 nucleotides downstream from the cut site. The PAM is a short DNA sequence (usually 2-6 base pairs in length) that follows the DNA region targeted for cleavage by the CRISPR system, such as CRISPR-Cas9.

In some embodiments, the PAM sequences, sgRNAs, or base editing tools targeting haplotypes or polymorphs of HLA loci does not include four Gs, four Cs, GC repeats, or combinations thereof.

In some embodiments, a CRISPR/Cas9 system specific to a donor's unique HLA haplotype can be developed by designing singular gRNAs targeting each of the donor-specific HLA-A, HLA-DPB1, and HLA-DQB1 genes (for example), using the gRNAs as described herein. To perform genetic knockout, the gRNA targets the Cas9 protein to the appropriate site to edit. Next, the Cas9 protein can perform a double strand break (DSB), where the DNA repairs through a non-homologous end joining (NHEJ) mechanism which generates indels resulting in a frameshift mutation and terminates the resulting protein's function. However, off-target genetic modifications can occur and alter the function of otherwise intact genes. For example, the Cas9 endonuclease can create DSBs at undesired off-target locations, even in the presence of some degree of mismatch. This off-target activity can create genome instability events, such as point mutations and genomic structural variations.

gRNAs can be used to develop clonal iPSCs from donor PBMCs. Such iPSC lines can be evaluated for (i) ON-target edit, (ii) OFF-target edits, and (iii) Translocation edits, for example using sequencing as described herein. Specifically, such assays can be performed by multiplex PCR with primers designed to target and enrich regions of interest followed by next-generation sequencing (e.g., Amplicon sequencing, AMP-seq). The ON-target panel and the translocation panel can amplify the intended edited region, allowing for selection of iPSC clones with the expected edits which are free from chromosomal translocation arising from unintended DSB cut-site fusion. The OFF-target panel can enrich any potential off-target regions identified via sequencing and allows for selection of iPSC clones with negligible off-target mutations. Together, these assays enable a screen of the iPSC clones to select the clones with the desired edits, while excluding potential CRISPR/Cas9-related genome integrity issues.

Although the differentiation potential of iPSCs has been demonstrated, some tissue-specific epigenetic memory from the starting material can interfere with iPSC differentiation. Therefore, in some embodiments, CD34+ cells can be selected for iPSC reprogramming, for example as described in Tobin S C and Kim K, “Generating pluripotent stem cells: differential epigenetic changes during cellular reprogramming,” FEBS Lett. 2012 August 31; 586 (18): 2874-81, hereby incorporated by reference in its entirety. In some embodiments, to create transgene and virus-free iPSCs, the CD34+ cells are electroporated with episomal vectors to reprogram them into iPSCs. For example, using oriP/EBNA1 vectors, the episomal vectors can contain 5 reprogramming factors (e.g., Oct4, Sox2, Lin28, Klf4, and L-Myc) and replicate extra-chromosomally only once per cell cycle and are completely cleared out once the iPSC reach approx. passage 5-10. These embodiments do not include transient p53 suppression to maintain important safeguard checkpoints and reduces the risk of selecting clones with genomic instability. To further ensure the genomic stability and integrity of reprogrammed iPSCs, genetic and genomic assays can be performed to select for clones which, for example, did not undergo translocation and mutation events, and that did not integrate the episomal vectors.

In some embodiments, whole-genome sequencing (WGS) is performed on CD34+ cells and on iPSC clones after reprogramming, where the genomes are compared for differences arising from editing. These analyses provide an assessment of which iPSC clone genomes differ from the CD34+ starting material, enabling informed selection iPSC clones which did not accrue mutations during the reprogramming.

In some embodiments, karyotyping analyses using systems such as KARYOSTAT assays is used to select iPSC clones which did not accrue indels and translocation during the reprogramming, for example as described in Ramme A P, et al, “Supporting dataset of two integration-free induced pluripotent stem cell lines from related human donors,” Data Brief. 2021 May 15; 37:107140, hereby incorporated by reference in its entirety. KARYOSTAT assays allow for visualization of chromosome aberrations with a resolution similar to G-banding karyotyping. The size of structural aberration that can be detected is >2 Mb for chromosomal gains and >1 Mb for chromosomal losses. The KARYOSTAT array is functionalized for balanced whole-genome coverage with a low-resolution DNA copy number analysis, where the assay covers all 36,000 RefSeq genes, including 14,000 OMIM targets. The assay enables the detection of aneuploidies, submicroscopic aberrations, and mosaic events.

In some embodiments, Array Comparative Genomic Hybridization (aCGH) analyses is used to select iPSC clones which did not accrue copy number aberrations (CNA) during reprogramming, for example as described in Wiesner et al. “Molecular Techniques,” Editor(s): Klaus J. Busam, Pedram Gerami, Richard A. Scolyer, “Pathology of Melanocytic Tumors,” Elsevier, 2019, pp. 364-373, ISBN 9780323374576; and Hussein S M, et al. “Copy number variation and selection during reprogramming to pluripotency,” Nature. 2011 Mar. 3; 471 (7336): 58-62, hereby incorporated by reference in its entirety. aCGH is a technique that analyzes the entire genome for CNA by comparing the sample DNA to reference DNA.

In some embodiments, targeted heme malignancy NGS panel analyses is used to select iPSC clones which did not accrue hematologic malignancy mutations during reprogramming. For example, targeted heme malignancy NGS panels can focus on myeloid leukemia, lymphoma, and/or other hematologic malignancy-associated genes to generate a smaller, more manageable data set than broader methods. Targeted heme malignancy NGS panel analysis includes the use of highly multiplexed PCR to amplify regions associated with hematologic malignancies followed by next-generation sequencing.

In some embodiments, Droplet Digital PCR (ddPCR) is used to select iPSC clones which did not integrate episomal vectors and that have been passaged enough for episomal vector clearance. As discussed herein, iPSC reprogramming of CD34+ cells can be achieved by delivering episomal vectors encoding reprogramming factors. However, episomal vectors can, albeit rarely, randomly integrate into the cellular genome, which could disrupt developmental processes, homeostasis, etc. Therefore, ddPCR methods can be used to detect residual episomal vector in the iPSC cultures and enable selection of iPSC clones which did not integrate episomal vectors.

In some embodiments, iPSCs which have undergone one or more of these analyses, and have indicated successful reprogramming, are used to build a pre-edited iPSC seed bank.

In some embodiments, gene editing is performed on the pre-edited iPSC seed bank, as described herein. Specifically, in embodiments, the Cas9 and gRNA which target each donor-specific HLA (e.g., HLA-A, HLA-DPB1, and HLA-DQB1) genes are electroporated into the iPSCs, allowing a recovery period of about 1 day to about 1 week. To ensure clonality (e.g, genetic homogeneity due to the cellular population arising from a single modified cell), a single-cell printer can be used to seed single cells into individual wells, for example in a 384-well plate. Such systems can be automated, require minimum-user interface, and ensure proof-of-clonality via imaging of each cell seeded in each well. After expanding the population from a single cell into a cellular colony, the subculture can be further expanded in culture trays with larger surface areas, e.g., into a 96-well plate, 12-well plate, etc., where a portion of the original clonal population of cells can be analyzed via on-target AMP-seq. Such analyses can guide the selection of clones bearing the desired edits. In some embodiments, a portion of cells with each expansion are used for Off-target AMP-seq and/or Translocation AMP-seq analyses to confirm genomic integrity throughout manipulation.

In some embodiments, after assessing that the selected clones are free from genomic aberrations related to the gRNA, the clones can be additionally tested for spontaneous mutations that might arise during expansion. For example, mutations affecting hematologic malignancy genes, indel, translocations, and CNA, e.g., as described for the pre-edited reprogrammed clones. Analyses for spontaneous mutations can include whole-genome sequencing (WGS), KARYOSTAT analysis, Array Comparative Genomic Hybridization (aCGH) analysis, targeted heme malignancy NGS panel AMP-Seq analysis, and/or Droplet Digital PCR (ddPCR).

In some embodiments, clones that are demonstrated to have preserved their genomic integrity are banked as a gene-edited iPSC seed bank.

In some embodiments, the cell line(s) are modified to express cytokines, suicide gene(s), T-cell receptor, single, dual, quad, and/or tandem chimeric antigen receptor (CAR), and/or combinations thereof.

In some embodiments, the cells (e.g., iPSCs or HSCs or progenitors or progenies thereof) of the present disclosure may be modified in a manner such that certain endogenously expressed genes, such as but not limited to genes encoding CCR5 or miR-155, or such that one or more genes encoding cell surface markers including but not limited to CD33, CLL, CD19, CD7, and/or CD38, are deleted or mutated to null their expression or such that they express non-functional proteins or are poorly expressed. Cell surface molecules that can be genetically modified according to this disclosure can be selected from any one of the cell surface molecules known to one of skill in the art, for example from CD1 through CD371, provided that genetic modification of the selected molecule or molecules provide the advantage of eliminating or ameliorating a harmful or toxic function in therapeutical applications of their wildtype counterpart (i.e., unmodified cells).

In various embodiments, the cell line(s) is inserted with CAR specific to myeloma, leukemia or lymphoma targets, including but not limited to CD19, CD33, BCMA, etc.

In various embodiments, the cell line(s) is inserted with tandem CARs, including but not limited CD38/IL3, CD20/CD19, etc.

In various embodiments, the cell line(s) is inserted with disease specific dual CAR, Quad CAR, tandem-CARs, etc.

In various embodiments, the cell line(s) is inserted with leucine-zipper system to incorporate multiple disease-modifying materials, including but not limited to dual CAR, Quad CAR, tandem-CAR, etc.

In various embodiments, the cell line(s) is inserted with leucine-zipper system to incorporate multiple disease-modifying materials.

Non-limiting examples include but are not limited to: (i) cell line(s) deleted for CCR5 to generate CCR5-deleted cellular therapies of HIV-AIDS patients; (2) cell line(s) deleted for CD33 to generate CD33-deleted cellular therapies for treating leukemia and/or lymphoma patients; (3) cell line(s) deleted for CD33 to generate CD33-deleted cellular therapies along with CAR-T, CAR-NK, CAR-T progenitor cells, or CAR-macrophage cells for treating leukemia and/or lymphoma patients.

In various embodiments, the cell line(s) is administered to mitigate the killing of normal cells or adverse effects caused by therapeutical applications of CAR-T therapy with CD33-specific CAR-T cells, with CD7-specific CAR-T cells, with CD8-specific CAR-T cells, with CD19-specific CAR-T cells, with CD20-specific CAR-T cells, with CD22-specific CAR-T cells, with CD123-specific CAR-T cells, with CD125-specific CAR-T cells, with CD133-specific CAR-T cells. with CD371-specific CAR-T cells or any CAR-NK, CAR-T or CAR-macrophage cells targeting the following tumor antigens:
(i) Human epidermal growth factor receptor 2 (HER2)-ovarian cancer, breast cancer, glioblastoma, colon cancer, osteosarcoma, and medulloblastoma;
(ii) Epidermal growth factor receptor (EGFR) positive malignancies, such as—non-small cell lung cancer, epithelial carcinoma, cholangiocarcinoma and glioma;
(iii) Mesothelin-mesothelioma, ovarian cancer, and pancreatic adenocarcinoma;
(iv) Prostate-specific membrane antigen (PSMA)-prostate cancer;
(v) Carcinoembryonic antigen (CEA)-pancreatic adenocarcinoma, breast cancer, and colorectal carcinoma;
(vi) Glypican-3-hepatocellular carcinoma;
(vii) Variant III of the epidermal growth factor receptor (EGFRvIII)—glioblastoma;
(viii) Disialoganglioside 2 (GD2)—neuroblastoma and melanoma;
(ix) Carbonic anhydrase IX (CAIX)—renal cell carcinoma;
(x) Interleukin-13Ra2—glioma;
(xi) Fibroblast activation protein (FAP)—malignant pleural mesothelioma;
(xii) L1 cell adhesion molecule (L1-CAM)—neuroblastoma, melanoma, and ovarian;
(xiii) Cancer antigen 125 (CA 125)—epithelial ovarian cancer;
(xiv) Cluster of differentiation 133 (CD 133)—glioblastoma and cholangiocarcinoma, adenocarcinoma;
(xv) Cancer/testis antigen 1B (CTAGIB)—melanoma and ovarian cancer;
(xvi) Mucin 1—seminal vesicle cancer; and
(xvii) Folate receptor-a (FR-a)—ovarian cancer.
(xviii) EGFRvIII—Glioblastoma.
(xix) Claudin 18.2-solid tumors, advanced gastric adenocarcinoma, pancreatic adenocarcinoma.
(xx) Mesothelin-mesothelioma, metastatic pancreatic, ovarian, cervical, lung.

See, for example, Zhou Z et al., Chimeric antigen receptor T cells applied to solid tumors. Front Immunol. 2022 Oct. 31; or Pooria et al, Novel antigens of CAR T cell therapy: New roads; old destination, Translational Oncology, Volume 14, Issue 7, 2021, each of which is incorporated herein by reference.

Alternatively, the cell populations can be used to generate cell therapies (e.g., HSCs or immune lineages) to be used with FDA approved CAR-T therapy, such as, Tisagenlecleucel, also known as tisa-cel (Kymriah), Axicabtagene ciloleucel, also known as axi-cel (Yescarta), Brexucabtagene autoleucel, also known as brexu-cel (Tecartus), Lisocabtagene maraleucel, also known as liso-cel (Breyanzi), Idecabtagene vicleucel, also known as ide-cel (Abecma), Ciltacabtegene autoleucel, also known as cilta-cel (Carvykti) or any other CAR-T therapy which damage the normal cells during their therapeutic applications.

In some embodiments, the pluripotent cells (e.g., iPSCs or HSCs or progenitors or progenies thereof) of the present disclosure can be further engineered by inserting at least one sequence encoding a transgene operatively linked to an endogenous or exogenous promoter, wherein the transgene is inserted within a genomic safe harbor locus. A genomic safe harbor (GSH) locus refers to a genetic locus that accommodates the insertion of exogenous DNA with either constitutive or conditional expression activity without significantly affecting the viability of somatic cells, progenitor cells, or germ line cells and ontogeny. Well known safe harbor loci include the AAVS1 adeno-associated virus insertion site on chromosome 19, the human homolog of the murine Rosa26 locus, and the CCR5 chemokine receptor gene. Tools and techniques for the insertion of transgene (i.e., the exogenous DNA) into safe harbor locus are well known to one of skill in the art, see for example Papapetrou E P et al. Gene Insertion Into Genomic Safe Harbors for Human Gene Therapy. Mol Ther. (2016) 678-84.

According to aspects and embodiments of this disclosure, a method is provided for making an HLA-modified cell of the disclosure using CRISPR-Cas9 gene editing. Exemplary sgRNAs are disclosed herein that can be used singly, or in some embodiments in combination to produce a plurality of edits (e.g., double strand breaks) in the target gene. The exemplary sgRNA are disclosed herein for generating deletions in exon 1 and/or exon 2 of various HLA genes (including within genomic coordinates shown in FIG. 11B). In the various embodiments, the method comprises contacting a cell with a Cas9 endonuclease (which can be delivered to the cell using any of the known processes) and one or more gRNAs (e.g., sgRNAs) targeting the Cas9 endonuclease to the HLA-specific or HLA allele-specific regions.

Tables 2A to 17 list spacer sequences useful for targeting HLA alleles as indicated, which can be incorporated into a gRNA (e.g., sgRNA) of a CRISPR-Cas9 system. In some embodiments, a sgRNA further comprises a scaffold sequence fused to the 3′ end of a spacer sequence. In some embodiments, a gRNA further comprises a tracr mate sequence fused to the 3′ end of a spacer sequence.

In some embodiments, the cell is homozygous or heterozygous for HLA-A*01:01, and the cell is contacted with one or two sgRNAs comprising a nucleotide sequence independently selected from Table 2A or Table 6.

In some embodiments, the cell is homozygous or heterozygous for HLA-A*02:05, and the cell is contacted with one or two sgRNAs comprising a nucleotide sequence independently selected from Table 2B.

In some embodiments, the cell is homozygous or heterozygous for HLA-A*03:01, and the cell is contacted with one or two sgRNAs comprising a nucleotide sequence independently selected from Table 2B or Table 6.

In some embodiments, the cell is homozygous or heterozygous for HLA-A*23:01, and the cell is contacted with one or two sgRNAs comprising a nucleotide sequence independently selected from Table 2C.

In some embodiments, the cell is homozygous or heterozygous for HLA-A*29:02, and the cell is contacted with one or two sgRNAs comprising a nucleotide sequence independently selected from Table 2E or Table 6.

In some embodiments, the cell is homozygous or heterozygous for HLA-A*25:01, and the cell is contacted with one or two sgRNAs comprising a nucleotide sequence independently selected from Table 2D.

In some embodiments, the cell is homozygous or heterozygous for HLA-A*33:01, and the cell is contacted with one or two sgRNAs comprising a nucleotide sequence independently selected from Table 2G or Table 6.

In some embodiments, the cell is homozygous or heterozygous for HLA-A*11:01, and the cell is contacted with one or two sgRNAs comprising a nucleotide sequence independently selected from Table 2C or Table 7.

In some embodiments, the cell is homozygous or heterozygous for HLA-A*26:01, and the cell is contacted with one or two sgRNAs comprising a nucleotide sequence independently selected from Table 2D or Table 7.

In some embodiments, the cell is homozygous or heterozygous for HLA-A*30:01, and the cell is contacted with one or two sgRNAs comprising a nucleotide sequence independently selected from Table 2E.

In some embodiments, the cell is homozygous or heterozygous for HLA-A*30:02, and the cell is contacted with one or two sgRNAs comprising a nucleotide sequence independently selected from Table 2F.

In some embodiments, the cell is homozygous or heterozygous for HLA-A*31:01, and the cell is contacted with one or two sgRNAs comprising a nucleotide sequence independently selected from Table 2F.

In some embodiments, the cell is homozygous or heterozygous for HLA-A*24:02, and the cell is contacted with one or two sgRNAs comprising a nucleotide sequence independently selected from Table 10.

In some embodiments, the cell is homozygous or heterozygous for DQB1*02:01, and the cell is contacted with one or two sgRNAs comprising a nucleotide sequence independently selected from Table 4A and Table 8.

In some embodiments, the cell is homozygous or heterozygous for DQB1*06:02, and the cell is contacted with one or two sgRNAs comprising a nucleotide sequence independently selected from Table 8.

In some embodiments, the cell is homozygous or heterozygous for DQB1*03:03, and the cell is contacted with one or two sgRNAs comprising a nucleotide sequence independently selected from Table 4B or Table 8.

In some embodiments, the cell is homozygous or heterozygous for DQB1*05:01, and the cell is contacted with one or two sgRNAs comprising a nucleotide sequence independently selected from Table 4C or Table 8.

In some embodiments, the cell is homozygous or heterozygous for DQB1*06:01, and the cell is contacted with one or two sgRNAs comprising a nucleotide sequence independently selected from Table 4D or Table 9.

In some embodiments, the cell is homozygous or heterozygous for DQB1*06:03, and the cell is contacted with one or two sgRNAs comprising a nucleotide sequence independently selected from Table 9.

In some embodiments, the cell is homozygous or heterozygous for DQB1*02:01, and the cell is contacted with one or two sgRNAs comprising a nucleotide sequence independently selected from Table 4A.

In some embodiments, the cell is homozygous or heterozygous for DQB1*03:01, and the cell is contacted with one or two sgRNAs comprising a nucleotide sequence independently selected from Table 4A.

In some embodiments, the cell is homozygous or heterozygous for DQB1*03:02, and the cell is contacted with one or two sgRNAs comprising a nucleotide sequence independently selected from Table 4B.

In some embodiments, the cell is homozygous or heterozygous for DQB1*05:03, and the cell is contacted with one or two sgRNAs comprising a nucleotide sequence independently selected from Table 4C.

In some embodiments, the cell is homozygous or heterozygous for DQB1*06:01, and the cell is contacted with one or two sgRNAs comprising a nucleotide sequence independently selected from Table 4D.

In some embodiments, the cell is homozygous or heterozygous for DQB1*06:04, and the cell is contacted with one or two sgRNAs comprising a nucleotide sequence independently selected from Table 4E.

In some embodiments, the cell is homozygous or heterozygous for HLA-B*07:02, and the cell is contacted with one or two sgRNAs comprising a nucleotide independently sequence selected from Table 3A or Table 11.

In some embodiments, the cell is homozygous or heterozygous for HLA-B*13:02, and the cell is contacted with one or two sgRNAs comprising a nucleotide sequence independently selected from Table 3B.

In some embodiments, the cell is homozygous or heterozygous for HLA-B*44:03, and the cell is contacted with one or two sgRNAs comprising a nucleotide sequence independently selected from Table 3F or Table 11.

In some embodiments, the cell is homozygous or heterozygous for HLA-B*18:01, and the cell is contacted with one or two sgRNAs comprising a nucleotide sequence independently selected from Table 3C.

In some embodiments, the cell is homozygous or heterozygous for HLA-B*57:01, and the cell is contacted with one or two sgRNAs comprising a nucleotide sequence independently selected from Table 3G or Table 11.

In some embodiments, the cell is homozygous or heterozygous for HLA-B*08:01, and the cell is contacted with one or two sgRNAs comprising a nucleotide sequence independently selected from Table 3A or Table 11.

In some embodiments, the cell is homozygous or heterozygous for HLA-B*40:01, and the cell is contacted with one or two sgRNAs comprising a nucleotide sequence independently selected from Table 3E.

In some embodiments, the cell is homozygous or heterozygous for HLA-B*14:02, and the cell is contacted with one or two sgRNAs comprising a nucleotide sequence independently selected from Table 3B or Table 12.

In some embodiments, the cell is homozygous or heterozygous for HLA-B*50:01, and the cell is contacted with one or two sgRNAs comprising a nucleotide sequence independently selected from Table 3F or Table 12.

In some embodiments, the cell is homozygous or heterozygous for HLA-B*37:01, and the cell is contacted with one or two sgRNAs comprising a nucleotide sequence independently selected from Table 12.

In some embodiments, the cell is homozygous or heterozygous for HLA-B*52:01, and the cell is contacted with one or two sgRNAs comprising a nucleotide sequence independently selected from Table 3G or Table 12.

In some embodiments, the cell is homozygous or heterozygous for HLA-B*38:01, and the cell is contacted with one or two sgRNAs comprising a nucleotide sequence independently selected from Table 3D or Table 13.

In some embodiments, the cell is homozygous or heterozygous for HLA-B*35:01, and the cell is contacted with one or two sgRNAs comprising a nucleotide sequence independently selected from Table 3D or Table 13.

In some embodiments, the cell is homozygous or heterozygous for DRB1*03:01, and the cell is contacted with one or two sgRNAs comprising a nucleotide sequence independently selected from Table 5A or Table 14.

In some embodiments, the cell is homozygous or heterozygous for DRB1*15:01, and the cell is contacted with one or two sgRNAs comprising a nucleotide sequence independently selected from Table 5F or Table 14.

In some embodiments, the cell is homozygous or heterozygous for DRB1*07:01, and the cell is contacted with one or two sgRNAs comprising a nucleotide sequence independently selected from Table 5C or Table 14.

In some embodiments, the cell is homozygous or heterozygous for DRB1*01:02, and the cell is contacted with one or two sgRNAs comprising a nucleotide sequence independently selected from Table 5A or Table 14.

In some embodiments, the cell is homozygous or heterozygous for DRB1*10:01, and the cell is contacted with one or two sgRNAs comprising a nucleotide sequence independently selected from Table 15.

In some embodiments, the cell is homozygous or heterozygous for DRB1*15:02, and the cell is contacted with one or two sgRNAs comprising a nucleotide sequence independently selected from Table 5F or Table 15.

In some embodiments, the cell is homozygous or heterozygous for DRB1*13:01, and the cell is contacted with one or two sgRNAs comprising a nucleotide sequence independently selected from Table 5D or Table 15.

In some embodiments, the cell is homozygous or heterozygous for DRB1*01:01, and the cell is contacted with one or two sgRNAs comprising a nucleotide sequence independently selected from Table 5A or Table 15.

In some embodiments, the cell is homozygous or heterozygous for DRB1*04:01, and the cell is contacted with one or two sgRNAs comprising a nucleotide sequence independently selected from Table 5B.

In some embodiments, the cell is homozygous or heterozygous for DRB1*04:02, and the cell is contacted with one or two sgRNAs comprising a nucleotide sequence independently selected from Table 5B.

In some embodiments, the cell is homozygous or heterozygous for DRB1*04:04, and the cell is contacted with one or two sgRNAs comprising a nucleotide sequence independently selected from Table 5C.

In some embodiments, the cell is homozygous or heterozygous for DRB1*11:04, and the cell is contacted with one or two sgRNAs comprising a nucleotide sequence independently selected from Table 5D.

In some embodiments, the cell is homozygous or heterozygous for DRB1*13:02, and the cell is contacted with one or two sgRNAs comprising a nucleotide sequence independently selected from Table 5E.

In some embodiments, the cell is homozygous or heterozygous for DRB1*14:01, and the cell is contacted with one or two sgRNAs comprising a nucleotide sequence independently selected from Table 5E.

In some embodiments, the cell is homozygous or heterozygous for HLA-DPB1*01:01, and the cell is contacted with one or two sgRNAs comprising a nucleotide sequence independently selected from Table 16.

In some embodiments, the cell is homozygous or heterozygous for HLA-DPB1*02:01, and the cell is contacted with one or two sgRNAs comprising a nucleotide sequence independently selected from Table 16.

In some embodiments, the cell is homozygous or heterozygous for HLA-DPB1*03:01, and the cell is contacted with one or two sgRNAs comprising a nucleotide sequence independently selected from Table 16.

In some embodiments, the cell is homozygous or heterozygous for HLA-DPB1*04:01, and the cell is contacted with one or two sgRNAs comprising a nucleotide sequence independently selected from Table 16.

In some embodiments, the cell is homozygous or heterozygous for HLA-DPB1*04:02, and the cell is contacted with one or two sgRNAs comprising a nucleotide sequence independently selected from Table 17.

In some embodiments, the cell is homozygous or heterozygous for HLA-DPB1*11:01, and the cell is contacted with one or two sgRNAs comprising a nucleotide sequence independently selected from Table 17.

In some embodiments, the cell is homozygous or heterozygous for HLA-DPB1*17:01, and the cell is contacted with one or two sgRNAs comprising a nucleotide sequence independently selected from Table 17.

In some embodiments, the cell is homozygous or heterozygous for HLA-DPB1*34:01, and the cell is contacted with one or two sgRNAs comprising a nucleotide sequence independently selected from Table 17.

In some embodiments, hiPSCs are used to generate embryoid bodies (EB), which can be used for generation of (i.e., isolation or enrichment of) CD34+ cells. For example, EBs can be dissociated, and the CD34+ hematopoietic precursors isolated or enriched. In some embodiments, human iPSC aggregates are expanded in a bioreactor as described, for example, in Abecasis B. et al., Expansion of 3D human induced pluripotent stem cell aggregates in bioreactors: Bioprocess intensification and scaling-up approaches. J. of Biotechnol. 246 (2017) 81-93.

Other bioreactors could include, but are not limited to, shear stress, mechanical strain and pulsed electromagnetic field bioreactors, large-scale stirred tank bioreactors, automated bioreactors, rotating wall bioreactors (RWBs), and rocking motions as seen with wave bioreactors, organ-on-chip bioreactors. Other bioreactor configurations can be employed that enable continuous, perfusion operation such as packed bed bioreactors (PBBs), fluidized bed bioreactors (FBBs), or PBBs and/or FBBs including the use of microcarriers, CultiBag bioreactors, and membrane bioreactors such as hollow fiber bioreactors (HFBs). Such bioreactors are contemplated for generating the pluripotent cells or progenitors or progenies derived therefrom of the present disclosure. Operation of the bioreactors may require coupling with an internal or external cell retention device on a recycle line, by centrifugation, sedimentation, ultrasonic separation or microfiltration with spin-filters, alternating tangential flow (ATF) filtration or tangential flow filtration (TFF).

In some embodiments, the process of generating cell populations comprising HSCs and/or HSPCs or progeny thereof can comprise generating CD34+-enriched cells from the differentiated pluripotent stem cells (e.g., EBs) and inducing endothelial-to-hematopoietic differentiation (e.g., for at least 2 days, but no more than 12 days). In some embodiments, HSCs comprising relatively high frequency of LT-HSCs can be generated from the cell populations using various stimuli or factors, including mechanical, biochemical, metabolic, and/or topographical stimuli, as well as factors such as extracellular matrix, niche factors, cell-extrinsic factors, induction of cell-intrinsic properties; and including pharmacological and/or genetic means.

In some embodiments, the method comprises preparing endothelial cells with hemogenic potential from pluripotent stem cells, prior to induction of EHT. In some embodiments, the combined over-expression of GATA2/ETV2, GATA2/TAL1, or ER71/GATA2/SCL can lead to the formation of endothelial cells with hemogenic potential from PSC sources. In some embodiments, the method comprises overexpression of E26 transformation-specific variant 2 (ETV2) transcription factor in the iPSCs. Following CD34+ enrichment, HSCs are then generated from the endothelial cells using mechanical, biochemical, pharmacological and/or genetic stimulation or modification. ETV2 can be expressed by introduction of an encoding non-integrating episomal plasmid, for constitutive or inducible expression of ETV2, and for production of transgene-free hemogenic ECs. In some embodiments, ETV2 is expressed from an mRNA introduced into the iPSCs. mRNA can be introduced using any available method, including electroporation or lipofection. Differentiation of cells expressing ETV2 can comprise addition of VEGF-A. See, Wang K, et al., Robust differentiation of human pluripotent stem cells into endothelial cells via temporal modulation of ETV2 with mRNA. Sci. Adv. Vol. 6 (2020). Cells generated in this manner may be used for producing CD34+ cells and inducing FHT according to embodiments of this disclosure. See PCT/US2021/062884, which is hereby incorporated by reference in its entirety.

In some embodiments, iPSC differentiation proceeds until cells are at least about 10% CD34+, or at least about 20% CD34+, or at least about 25% CD34+, or at least about 30% CD34+, or at least 40% CD34+. In some embodiments, CD34+ enrichment and EHT may be induced at Day 6 to Day 14 of iPSC differentiation, such as for example, Day 7, Day 8, Day 9, Day 10, Day 11, Day 12, Day 13, or Day 14. Differentiation of iPSCs can be according to known techniques. In some embodiments, iPSC differentiation involves factors such as, but not limited to, combinations of bFGF, Y27632, BMP4, VEGF, SCF, EPO, TPO, IL-6, IL-11, and/or IGF-1. In some embodiments, hPSCs are differentiated using feeder-free, serum-free, and/or GMP-compatible materials. In some embodiments, hPSCs are co-cultured with murine bone marrow-derived feeder cells such as OP9 or MS5 or mouse embryonic fibroblast cell line in serum-containing medium. The culture can contain growth factors and cytokines to support differentiation of embryoid bodies or monolayer system. The OP9 co-culture system can be used to generate multipotent HSPCs, which can be differentiated further to several hematopoietic lineages including T lymphocytes, B lymphocytes, megakaryocytes, monocytes or macrophages, and erythrocytes. See Netsrithong R. et al., Multilineage differentiation potential of hematoendothelial progenitors derived from human induced pluripotent stem cells, Stem Cell Research & Therapy Vol. 11 Art. 481 (2020). Alternatively, a step-wise process using defined conditions with specific signals can be used. For example, the expression of HOXA9, ERG, RORA, SOX4, and MYB in human PSCs favors the direct differentiation into CD34+/CD45+ progenitors with multilineage potential. Further, expression of factors such as HOXB4, CDX4, SCL/TAL1, or RUNX1a support the hematopoietic program in human PSCs. See Doulatov S. et al., Induction of multipotential hematopoietic progenitors from human pluripotent stem cells via re-specification of lineage-restricted precursors, Cell Stem Cell. 2013 Oct. 3; 13 (4).

Induction of EHT can be with any known process. In some embodiments, induction of EHT generates an HSC population comprising LT-HSCs. In some embodiments, EHT generates a cell population comprising HSPCs. In some embodiments, EHT generates HSCs and/or HSPCs through endothelial or hemogenic endothelial cell (HEC) precursors using mechanical, biochemical, pharmacological and/or genetic means (e.g., via stimulation, inhibition, and/or genetic modifications). In some embodiments, the EHT generates a stem cell population comprising one or more of long-term hematopoietic stem cells (LT-HSCs), short-term hematopoietic stem cells (ST-HSCs), and HSPCs. In various embodiments, EHT can be induced in the culture for from 2 days to 12 days, such as about 4 days to about 8 days (e.g., about 4 days, about 5 days, about 6 days, about 7 days, or about 8 days). In some embodiments, EHT is induced in the culture from about 5 days to about 7 days.

In some embodiments, the HSC and/or HSPC population or fraction thereof is differentiated to T cells or progenitors or derivatives thereof independent of the use of an agonist of a mechanosensitive receptor or a mechanosensitive channel, such as Yoda1. In some embodiments, the use of an agonist of a mechanosensitive receptor or a mechanosensitive channel (e.g., Yoda1) is optional. Thus, in some embodiments, CD34+ cells are enriched from a differentiated pluripotent stem cell population to prepare a CD34+-enriched population. Endothelial-to-hematopoietic transition of the CD34+-enriched cell population is induced for at least two days, but no more than 12 days in which the use of an agonist of a mechanosensitive receptor or a mechanosensitive channel such as Yoda1, jedi1, jedi2, or ssRNA40 is optional. The HSCs and/or HSPCs are differentiated to a progenitor T cell population or a T cell population (e.g., as described herein). In some embodiments, the endothelial-to-hematopoietic transition of the CD34+-enriched cell population is induced for at least for two days, and further for about 4 hours, or about 8 hours, or about 12 hours, or about 16 hours, or about 20 hours, or about 24 hours, or about 2 days, or about 3 days, or about 4 days, or about 5 days, or about 6 days, or about 7 days, or about 8 days, or about 9 days, or about 10 days. The total EHT differentiation proceeds for no more than 12 days.

In various embodiments, CD34+ cells (e.g., the floater and/or adherent cells) are harvested from the culture undergoing endothelial-to-hematopoietic transition, such as between Day 10 to Day 20 of iPSC differentiation, such as from Day 10 to Day 17, or from Day 12 to Day 15 of iPSC differentiation.

In some embodiments, the method comprises increasing the expression or activity of dnmt3b in PSCs, embryoid bodies, CD34-enriched cells, ECs, HECs or HSCs, which can be by mechanical, genetic, biochemical, or pharmacological means. In some embodiments, the method comprises increasing activity or expression of DNA (cytosine-5−)-methyltransferase 3 beta (Dnmt3b) and/or GTPase IMAP Family Member 6 (Gimap6) in the cells. See WO 2019/236943 and WO 2021/119061, which is hereby incorporated by reference in its entirety. In some embodiments, the induction of EHT comprises increasing the expression or activity of dnmt3b.

In some embodiments, cells are contacted with an effective amount of an agent such as but limited to an agonist of a mechanosensitive receptor or a mechanosensitive channel that increases the activity or expression of Dnmt3b. In some embodiments, the mechanosensitive receptor is Piezol. An exemplary Piezol agonist is Yoda1.

In some embodiments, the mechanosensitive receptor is Trpv4. An exemplary Trpv4 agonist is GSK1016790A. Yoda1 (2-[5-[[(2,6-Dichlorophenyl)methyl]thio]-1,3,4-thiadiazol-2-yl]-pyrazine) is a small molecule agonist developed for the mechanosensitive ion channel Piezol. Syeda R, Chemical activation of the mechanotransduction channel Piezol. eLife (2015).

Yoda 1 has the following structure:

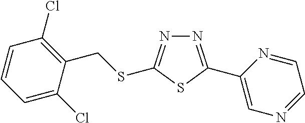 [SMILES: Clc1cccc(Cl)c1CSc1nnc(-c2cnccn2)s1]

Derivatives of Yoda1 can be employed in various embodiments. For example, derivatives comprising a 2,6-dichlorophenyl core are employed in some embodiments. Exemplary agonists are disclosed in Evans E L, et al., Yoda1 analogue (Dooku1) which antagonizes Yoda1-evoked activation of Piezol and aortic relaxation, British J. of Pharmacology 175 (1744-1759): 2018. Still other Piezol agonist include Jedi1, Jedi2, single-stranded (ss) RNA (e.g., ssRNA40) and derivatives and analogues thereof. See Wang Y., et al., A lever-like transduction pathway for long-distance chemical-and mechano-gating of the mechanosensitive Piezol channel. Nature Communications (2018) 9:1300; Sugisawa, et al., RNA Sensing by Gut Piezol Is Essential for Systemic Serotonin Synthesis, Cell, Volume 182, Issue 3, 2020, Pages 609-624, which are hereby incorporated by reference in their entireties. These Piezol agonists are commercially available. In various embodiments, the effective amount of the Piezol agonist or derivative is in the range of about 1 μM to about 500 μM, or about 5 μM to about 200 μM, or about 5 μM to about 100 μM, or in some embodiments, in the range of about 25 μM to about 150 μM, or about 25 μM to about 100 μM, or about 25 μM to about 50 μM. Alternatively, single-stranded (ss) RNA (e.g., ssRNA40), and derivatives and analogues thereof, can be used for Piezol activation.

In various embodiments, pharmacological Piezol activation is applied to CD34+ cells (i.e., CD34-enriched cells). In certain embodiments, pharmacological Piezol activation may further be applied to iPSCs, embryoid bodies, ECs, hemogenic endothelial cells (HECs), HSCs, hematopoietic progenitors, as well as hematopoietic lineage(s). In certain embodiments, Piezol activation is applied at least to EBs generated from iPSCs, CD34+ cells isolated from EBs, and/or combinations thereof. The use of Piezol activation for generation of HSCs or progeny thereof is described in US 2021/0222125 and US 2022/00049221, which are hereby incorporated by reference in their entireties.

Alternatively, or in addition, the activity or expression of Dnmt3b can be increased directly in the cells, e.g., in CD34-enriched cells. For example, mRNA expression of Dnmt3b can be increased by delivering Dnmt3b-encoding transcripts to the cells, or by introducing a Dnmt3b-encoding transgene, or a transgene-free method, not limited to introducing a non-integrating episome to the cells. In some embodiments, gene editing is employed to introduce a genetic modification to Dnmt3b expression elements in the cells, such as, but not limited to, to increase promoter strength, ribosome binding, RNA stability, and/or impact RNA splicing.

In some embodiments, the method comprises increasing the activity or expression of Gimap6 in the cells, alone or in combination with Dnmt3b and/or other genes that are up- or down regulated upon cyclic strain or Piezol activation. To increase activity or expression of Gimap6, Gimap6-encoding mRNA transcripts can be introduced to the cells, transgene-free approaches can also be employed, including but not limited, to introducing an episome to the cells; or alternatively a Gimap6-encoding transgene. In some embodiments, gene editing is employed to introduce a genetic modification to Gimap6 expression elements in the cells (such as one or more modifications to increase promoter strength, ribosome binding, RNA stability, or to impact RNA splicing).

In embodiments of this disclosure employing mRNA delivery to cells, known chemical modifications can be used to avoid the innate-immune response in the cells. For example, synthetic RNA comprising only canonical nucleotides can bind to pattern recognition receptors, and can trigger a potent immune response in cells. This response can result in translation block, the secretion of inflammatory cytokines, and cell death. RNA comprising certain non-canonical nucleotides can evade detection by the innate immune system, and can be translated at high efficiency into protein. See U.S. Pat. No. 9,181,319, which is hereby incorporated by reference, particularly with regard to nucleotide modification to avoid an innate immune response.

In some embodiments, expression of Dnmt3b and/or Gimap6 is increased by introducing a transgene into the cells, which can direct a desired level of overexpression (with various promoter strengths or other selection of expression control elements). Transgenes can be introduced using various viral vectors or transfection reagents (including Lipid Nanoparticles) as are known in the art. In some embodiments, expression of Dnmt3b and/or Gimap6 is increased by a transgene-free method (e.g., episome delivery or lipid nanoparticle with mRNA). In some embodiments, expression or activity of Dnmt3b and/or Gimap6 or other genes disclosed herein are increased using a gene editing technology, for example, to introduce one or more modifications to increase promoter strength, ribosome binding, or RNA stability.

In some embodiments, the method comprises applying cyclic 2D, 3D, or 4D stretch to cells. In various embodiments, the cells subjected to cyclic 2D, 3D, or 4D stretch are selected from one or more of CD34-enriched cells, iPSCs, ECs, and HECs. For example, a cell population is introduced to a bioreactor that provides a cyclic strain, or a biomechanical stretching or a cyclic-strain biomechanical stretching. Cyclic-strain biomechanical stretching is described in WO 2017/096215, which is hereby incorporated by reference in its entirety. The cyclic strain, or biomechanical stretching or cyclic-strain biomechanical stretching can increase the activity or expression of Dnmt3b and/or Gimap6. In these embodiments, mechanical means apply stretching forces to the cells, or to a cell culture surface having the cells (e.g., ECs or HECs) cultured thereon. For example, a computer controlled vacuum pump system or other means for providing a stretching force (e.g., the FlexCell™ Tension System, the Cytostretcher System) attached to flexible biocompatible and/or biomimetic surface can be used to apply cyclic 2D, 3D, or 4D stretch ex vivo to cells under defined and controlled cyclic strain conditions. For example, the applied cyclic stretch can be from about 1% to about 20% cyclic strain (e.g., about 6% cyclic strain) for several hours or days (e.g., about 7 days). In various embodiments, cyclic strain or mechanical stretching is applied for at least about one hour, at least about two hours, at least about six hours, at least about eight hours, at least about 12 hours, at least about 24 hours, at least about 48 hours, at least about 72 hours, at least about 96 hours, at least about 120 hours, at least about 144 hours, or at least about 168 hours.

In various embodiments, cyclic strain or mechanical stretching is applied for at least for a period ranging between about one hour to about two hours, at least for a period ranging between about six hours to about eight hours, at least for a period ranging between about 12 hours to about 24 hours, at least for a period ranging between about 48 hours to about 72 hours, at least for a period ranging between about 96 hours to about 120 hours, at least for a period ranging between about 144 hours to about 168 hours.

Alternatively or in addition, EHT is stimulated by Trpv4 activation. The Trpv4 activation can be by contacting cells (e.g., CD34-enriched cells, ECs, or HECs) with one or more Trpv4 agonists, which are optionally selected from GSK1016790A, 4alpha-PDD, or analogues and/or derivatives thereof.

In some embodiments, cells are contacted with an effective amount of an agent that (a) modulates histone acetylation; or (b) modulates histone methylation; or (c) modulates TGF beta signaling; or (d) modulates wnt and/or notch signaling pathway.

Modulating agents can be selected from inhibitors which modulate signaling through TGF beta pathway, wnt pathway, notch pathway or modulate histone methylation and/or acetylation. Some of the proteins that are known to be acetylated include p53, HSP90, tubulin, NF-κB, HIF-1α, RUNX3, STAT-3, E2F1, Ku70 and c-MYC. Acetylation functions as a broad post-translational modification regulating protein functions including DNA-binding, activity of transcription factors, subcellular localization, and protein stability.

Nonlimiting examples of modulating agents include but are not limited to inhibitor of histone methyltransferase EZH1, DNA methyltransferase inhibitors (DNMTi), histone deacetylase inhibitors (HDACi), Suberoylanilide bis-hydroxamic acid (SBHA), Tranylcypromine, LSD1 inhibitors, such as IV RN-1, LSD1-C76, LSD1 inhibitor II S2101, LSD1 inhibitor III CBB1007, LSD1 inhibitor I, SNDX-275 (MS-275, Entinostat), CI-994 (Tacedinaline), MGCD-0103, Valproic acid (VPA), Sodium butyrate, Phenyl butyrate (S-HDAC-42, AR-42), Depsipeptide (Romidepsin), Apicidin, JNJ-26481585, Suberoylanilide hydroxamic acid (SAHA; Vorinostat), NVP-LAQ824 (Dacinostat), CR-2408, RAS2410 (Resminostat), Trichostatin-A (TSA), LBH589 (Panobinostat), ITF2357 (Gavinostat), PXD101 (Belinostat), ACY-1215 (Rocilinostat), KD5170, and Tubacin.

Several TGF-β kinase inhibitors have been designed to bind the ATP-binding domain of TGF-β R kinase and inhibit ATP kinase activity and block the downstream signaling cascade. TGF-β inhibitors can be selected from one or more of Galunisertib (LY21557299), ALK5 inhibitor II (E-616452), LY364947, A83-01, and DMH1, LY573636 (Tasisulam), LY2109761, LY364937, Ki26894, LY580276, SB-431542, SB-505124, A83-01, SD-093 and SD-208, IN-1130, and Vactosertib (TEW-7197). TGF-β inhibitors could also include antibodies, such as but not limited to SRK181-mlgG1, Fresolimumab, LY3022859, 264RAD, 1D11, 2G7, or a pyrimidoindole derivative including, for example, UM171 or UM729.

In certain cases, the agent includes a compound that inhibits a protein that propagates p38 signaling, such as SB203580. In additional embodiments, the one or more agents include a compound that inhibits a protein that promotes beta-catenin degradation selected from one or more of lithium chloride, CHIR99021, ICG-001, XAV939, pyrvinium, BIO, C2 inhibitor, CRT-3, -5 and -14, stapled peptide StAx35R, and FGF2 or a recombinant version thereof.

In some embodiments the method employs an allosteric agonist, including but not limited to yoda 1, jedi 1, jedi 2, docosahexaenoic acid or analogs thereof or any agonist that modulates the activity of the mechanosensitive Piezo channels.

In a non-limiting example, the iPSC cell line(s)-derived HSCs are generated by CD34-enrichment from embryonic bodies and endothelial-to-hematopoietic transition that is induced at Day 8 to Day 15 of iPSC differentiation. CD34-cells are harvested from culture undergoing endothelial-to-hematopoietic transition, including harvesting of CD34+ floater and/or adherent cells. In some embodiments, the induction of endothelial-to-hematopoietic transition comprises increasing the expression or activity of dnmt3b.

In various embodiments, the induction of endothelial-to-hematopoietic transition comprises applying cyclic stretch to the CD34-enriched population, where the cyclic stretch is optionally 2D, 3D, or 4D cyclic stretch. Alternatively or in addition, the induction of endothelial-to-hematopoietic transition comprises Piezol activation. The Piezol activation can be achieved by contacting the CD34-enriched cells or fraction thereof with one or more Piezol agonists, which are optionally selected from Yoda1, Jedi1, Jedi2, or analogues or derivatives thereof. In some embodiments, the induction of endothelial-to-hematopoietic transition comprises Trpv4 activation, wherein the Trpv4 activation is optionally by contacting the CD34-enriched cells with one or more Trpv4 agonists, which are optionally selected from GSK1016790A, 4alpha-PDD, or analogues or derivatives thereof. Optionally any of the modulating agents provided in the disclosure may be used in combination with any other agent(s) disclosed herein.

It is contemplated that the one or more agents can be added to act concomitantly or to act on the same or different pathways. For example, they could simultaneously act as inhibitors of TGF-beta or they can act independently to inhibit histone demethylase and TGF-beta respectively, which could be simultaneous or sequential.

Where cell populations or banks are described herein as having a certain phenotype it is understood that the phenotype represents a significant portion of the cell population, such as at least 25%, at least 40%, or at least about 50%, or at least about 60%, or at least about 75%, or at least about 80%, or at least about 90% of the cell population. Further, at various steps, cell populations can be enriched for cells of a desired phenotype, and/or depleted of cells of an undesired phenotype, such that cell population comprise at least about 75%, or at least about 80%, or at least about 90% of the desired phenotype. Such positive and negative selection methods are known in the art. For example, cells can be sorted based on cell surface antigens (including those described herein) using a fluorescence activated cell sorter, or magnetic beads which bind cells with certain cell surface antigens. Negative selection columns can be used to remove cells expressing undesired cell-surface markers. In some embodiments, cells are enriched for CD34+ cells (prior to and/or after undergoing EHT). In some embodiments, the cell population is cultured under conditions that promote expansion of CD34+ cells to thereby produce an expanded population of stem cells.

In various embodiments, CD34+ cells (e.g., the floater and/or adherent cells) are harvested from the culture undergoing endothelial-to-hematopoietic transition between Day 8 to Day 15 of iPSC differentiation.

In various embodiments, the HSCs or CD34-enriched cells are further expanded. For example, the HSCs or CD34-enriched cells can be expanded according to methods disclosed in U.S. Pat. Nos. 8,168,428; 9,028,811; 10,272,110; and 10,278,990, which are hereby incorporated by reference in their entireties. In some embodiments, ex vivo expansion of HSCs or CD34-enriched cells employs prostaglandin E2 (PGE2) or a PGE2 derivative. In some embodiments of this disclosure, the HSCs comprise at least about 0.01% LT-HSCs, or at least about 0.05% LT-HSCs, or at least about 0.1% LT-HSCs, or at least about 0.5% LT-HSCs, or at least about 1% LT-HSCs.

Hematopoietic stem cells (HSCs) which give rise to erythroid, myeloid, and lymphoid lineages, can be identified based on the expression of CD34 and the absence of lineage specific markers (termed Lin−). In some embodiments, a population of stem cells comprising HSCs are enriched, for example, as described in U.S. Pat. No. 9,834,754, which is hereby incorporated by reference in its entirety. For example, this process can comprise sorting a cell population based on expression of one or more of CD34, CD90, CD45, CD38, and CD43. A fraction can be selected for further differentiation that is one or more of CD34⁻, CD90⁺ and/or CD45+, CD38⁻, and CD43⁻. In some embodiments, the stem cell population for differentiation to a hematopoietic lineage is at least about 80% CD34⁺, or at least about 90% CD34⁺, or at least about 95% CD34⁺.

In some embodiments, multi-potent hematopoietic stem cells (HSCs) self-renew and differentiate into two types of progenitor cells with specific lineage commitments. Like HSCs, human lineage-restricted progenitor cells also express CD34 and Flt-3/Flk-2. Myeloid progenitors (MPs), in human and mouse, express IL-3 R alpha and give rise to cells of the myeloid lineage including megakaryocytes, erythrocytes, granulocytes, and macrophages. Lymphoid progenitors (LPs) are cells that develop from HSCs and give rise to B cells, T cells, and Natural Killer (NK) cells. Human bone marrow LPs are CD34+CD38+ Neprilysin+, and cord blood CLPs are CD34+CD38-CD7+.

In some embodiments, the cell populations are differentiated to hematopoietic lineage cells for administration to a recipient. In various embodiments, the hematopoietic lineage cells are selected from common lymphoid precursor (CLP) cells, granulocyte-monocyte progenitor (GMP) cells, progenitor-T cells, T lymphocytes, B lymphocytes, Natural Killer cells, neutrophils, monocyte, macrophages, red cells, megakaryocytes, and platelets. In some aspects and embodiments, the disclosure provides a method for generating a CD7+ progenitor T cell population, or a derivative of this population. For example, the method comprises generating a hematopoietic stem cell (HSC) population comprising human long-term hematopoietic stem cells (LT-HSCs) from iPSCs (e.g., hiPSCs). The HSC population is derived by induction of endothelial-to-hematopoietic transition of CD34+ cells (e.g., CD34+ cells derived from embryoid bodies). The HSC population (or cells isolated therefrom) is cultured with a partial or full Notch ligand, sonic hedgehog (SHH), RetroNectin (or other extracellular matrix component(s)), and/or combinations thereof, to produce a population comprising CD7+ progenitor T cells or a derivative cell population.

The Notch signaling pathway regulates the formation, differentiation, and function of progenitor T-cells, pre-T cells, and/or mature T lymphocytes. In vivo, T cell development proceeds after lymphocyte progenitors differentiate from bone marrow hematopoietic stem cells and migrate to the thymus. Specialized thymic epithelial cells induce T cells to develop along a controlled pathway. Notch signaling plays a critical role during T lineage commitment in the thymus. As lymphoid progenitors enter the thymus, they encounter dense expression of Notch ligands on thymic epithelium that drives thymopoiesis. The present disclosure provides HSC populations generated ex vivo from iPSCs and which respond to Notch ligand, SHH, and/or component(s) of extracellular matrix, by robust production of T progenitor cells and T cell lineages ex vivo.

In some embodiments, the stem cell population, or CD34-enriched cells or fraction thereof, or derivative population are expanded as described in US 2020/0308540, which is hereby incorporated by reference in its entirety. For example, the cells are expanded by exposing the cells to an aryl hydrocarbon receptor antagonist including, for example, SR1 or an SR1-derivative. See also, Wagner et al., Cell Stem Cell 2016; 18 (1): 144-55 and Boitano A., et al., Aryl Hydrocarbon Receptor Antagonists Promote the Expansion of Human Hematopoietic Stem Cells. Science 2010 Sep. 10; 329 (5997): 1345-1348.

In some embodiments, the compound that promotes expansion of CD34⁺ cells includes a pyrimidoindole derivative including, for example, UM171 or UM729 (see US 2020/0308540, which is hereby incorporated by reference).

In some embodiments, the stem cell population or CD34-enriched cells are further enriched for cells that express Periostin and/or Platelet Derived Growth Factor Receptor Alpha (pdgfra) or are modified to express Periostin and/or pdgfra, as described in WO 2020/205969 (which is hereby incorporated by reference in its entirety). Such expression can be by delivering encoding transcripts to the cells, or by introducing an encoding transgene, or a transgene-free method, not limited to introducing a non-integrating episome to the cells. In some embodiments, gene editing is employed to introduce a genetic modification to expression elements in the cells, such as to modify promoter activity or strength, ribosome binding, RNA stability, or impact RNA splicing.

In still other embodiments, the stem cell population or CD34-enriched cells are cultured with an inhibitor of histone methyltransferase EZH1. Alternatively, EZH1 is partially or completely deleted or inactivated or is transiently silenced in the stem cell population. Inhibition of EZH1 can direct myeloid progenitor cells (e.g., CD34+CD45+) to lymphoid lineages. See WO 2018/048828, which is hereby incorporated by reference in its entirety. In still other embodiments, EZH1 is overexpressed in the stem cell population.

In various embodiments, the cell lines developed from primary cells or the cell lines derived from iPSC cell line or banks thereof are hematopoietic lineage cells. The hematopoietic lineage is selected from common lymphoid precursor (CLP) cells, granulocyte-monocyte progenitor (GMP) cells, progenitor-T cells, T lymphocytes, B lymphocytes, Natural Killer cells, neutrophils, monocyte, macrophages, red cells, megakaryocytes, and platelets.

In some embodiments, the HSC population or fraction thereof is differentiated ex vivo to progenitor T cells, T cells, NK cells, and/or fractions or analogous thereof.

In some embodiments, the HSC population or fraction thereof is cultured with a partial or full Notch ligand to produce a population comprising CD7+ progenitor T cells or a derivative cell population.

In some embodiments, the derivative cell population is a T cell, precursors, subtypes, and derivatives of T cells, or NK cell population.

In some embodiments, the cell population is cultured with a Notch ligand, partial or full, SHH, extracellular matrix component(s), and/or combinations thereof, ex vivo, to differentiate HSCs to CD7⁺ progenitor T cells, and optionally to a T cell lineage or other lineage (e.g., NK cell). Further, according to known processes, xenogenic OP9-DLI cells are often employed for differentiation to T cells. The OP9-DLI co-culture system uses a bone marrow stromal cell line (OP9) transduced with the Notch ligand delta-like-1 (DLL1) to support T cell development from stem cell sources. The OP9-DLI system limits the potential of the cells for clinical application. There is a need for feeder-cell-free systems that can generate T lymphocytes from hiPSCs for clinical use, and in some embodiments the present disclosure meets this objective.

The term “Notch ligand” as used herein refers to a ligand capable of binding to a Notch receptor polypeptide present in the membrane of a hematopoietic stem cell or progenitor T cell. The Notch receptors include Notch-1, Notch-2, Notch-3, and Notch-4. Notch ligands typically have a DSL domain (D-Delta, S-Serrate, and L-Lag2) comprising 20-22 amino acids at the amino terminus, and from 3 to 8 EGF repeats on the extracellular surface. In various embodiments, the Notch ligand comprises at least one of Delta-Like-1 (DLL1), Delta-Like-4 (DLL4), SFIP3, and Delta^{Max} (disclosed in PCT/US2020/041765 and PCT/US2020/030977, which are hereby incorporated by reference in their entirety) or a functional portion thereof. A key signal that is delivered to incoming lymphocyte progenitors by the thymus stromal cells in vivo is mediated by DL4, which is expressed by cortical thymic epithelial cells.

The earliest intrathymic progenitors express high levels of CD34 and CD7, do not express CD1a, and are triple-negative (TN) for mature T cell markers: CD4, CD8, and CD3. Commitment to the T cell lineage is associated with the expression of CD1a by CD7-expressing pro-thymocytes. Thus, immature stages of T-cell development are typically delineated as CD34⁺CD1a⁻ (most immature) and CD34⁺CD1a⁺ cells. The transition from CD34⁺CD7⁺CD1a⁻ to CD34⁺CD7⁺CD1a⁺ by early thymocytes is associated with T-cell commitment. CD34⁺CD7⁺CD1a⁺ cells are likely T-lineage restricted. Following this stage, thymocytes progress to a CD4 immature single positive stage, at which point CD4 is expressed in the absence of CD8. Thereafter, a subset of the cells differentiates to the CD4⁺CD8⁺ double positive (DP) stage. Finally, following TCRα rearrangement, TCRαβ-expressing DP thymocytes undergo positive and negative selection, and yield CD4⁺CD8⁻ and CD4⁻CD8⁺ single positive (SP) T-cells.

In some embodiments, progenitor T cells are isolated by enrichment for CD7 expression. In some embodiments, progenitor T cells are expanded as described in US 2020/0308540, which is hereby incorporated by reference in its entirety. For example, the cells may be expanded by exposing the cells to an aryl hydrocarbon receptor antagonist including, for example, SR1 or an SR1-derivative. See also, Wagner et al., Cell Stem Cell 2016; 18 (1): 144-55. In some embodiments, the compound that promotes expansion includes a pyrimidoindole derivative including, for example, UM171 or UM729 (see US 2020/0308540, which is hereby incorporated by reference).

Differentiation to progenitor T cells can further include in some embodiments the presence of stem cell factor (SCF), Flt3L and interleukin (IL)-7. In various embodiments, CD7+ progenitor T cells created express CD1a. The CD7+ progenitor T cells do not express CD34 or express a diminished level of CD34 compared to the HSC population. In some embodiments, the CD7+ progenitor T cells (or a portion thereof) further express CD5. Accordingly, the phenotype of the progenitor T cells may be CD7⁺CD1a⁺. In some embodiments, the phenotype of the progenitor T cells is CD7⁺CD5⁺. In some embodiments, the progenitor T cells are CD7⁺CD1a⁺CD5⁺, and optionally CD34⁺. In some embodiments, the progenitor T cells are CD7⁺CD1a⁻CD5, and optionally CD34⁺.

In some embodiments, the progenitor T cells exhibit a diminished level of CD34 expression, minimal CD34 expression (compared to the HSC population), or no CD34 expression. In some embodiments, CD34 expression is diminished in the population by at least about 50%, or at least about 75%, relative to the HSC population.

In some embodiments, the Notch ligand is an anti-Notch (agonistic) antibody that can bind and engage Notch signaling. In some embodiments, the antibody is a monoclonal antibody (including a human or humanized antibody), a single chain antibody (scFv), a nanobody, or other antibody fragment or antigen-binding molecule capable of activating the Notch signaling pathway.

In some embodiments, the Notch ligand is a Delta family Notch ligand. The Delta family ligand in some embodiments is Delta-1 (Genbank Accession No. AF003522, Homo sapiens), Delta-like 1 (DLL1, Genbank Accession No. NM_005618 and NP_005609, Homo sapiens; Genbank Accession No. X80903, 148324, M. musculus), Delta-4 (Genbank Accession No. AF273454, BAB18580, Mus musculus; Genbank Accession No. AF279305, AAF81912, Homo sapiens), and/or Delta-like 4 (DLL4; Genbank Accession. No. Q9NR61, AAF76427, AF253468, NM_019074, Homo sapiens; Genbank Accession No. NM 019454, Mus musculus). Notch ligands are commercially available or can be produced, for example, by recombinant DNA techniques.

In some embodiments, the Notch ligand comprises an amino acid sequence that is at least about 70%, or at least about 80%, or at least about 90%, or at least about 95%, or at least about 97% identical (e.g., about 100% identical) to human DLL1 or DLL4 Notch ligand. Functional derivatives of Notch ligands (including fragments or portions thereof) will be capable of binding to and activating a Notch receptor. Binding to a Notch receptor may be determined by a variety of methods known in the art including in vitro binding assays and receptor activation/cell signaling assays.

In some embodiments, the Notch ligand is a DLL4 having one or more affinity enhancing mutations, such as one or more (or all) of: G28S, F107L, 1143F, H194Y, L206P, N257P, T271L, F280Y, S301R and Q305P, with respect to hDLL4. See Gonzalez-Perez, et al., Affinity-matured DLL4 ligands as broad-spectrum modulators of Notch signaling, Nature Chemical Biology (2022).

In various embodiments, the Notch ligands are soluble, and are optionally immobilized on microparticles or nanoparticles, which are optionally paramagnetic to allow for magnetic enrichment or concentration processes. In still other embodiments, the Notch ligands are immobilized on a 2D or 3D culture surface, optionally with other adhesion molecules such as VCAM-1. See US 2020/0399599, which is hereby incorporated by reference in its entirety. In other embodiments, the beads or particles are polymeric (e.g., polystyrene or PLGA), gold, iron dextran, or constructed of biological materials, such as particles formed from lipids and/or proteins. In various embodiments, the particle has a diameter or largest dimension of from about 0.01 μm (10 nm) to about 500 μm (e.g., from about 1 μm to about 7 μm). In still other embodiments, polymeric scaffolds with conjugated ligands can be employed, as described in WO 2020/131582, which is hereby incorporated by reference in its entirety. For example, scaffold can be constructed of polylactic acid, polyglycolic acid, PLGA, alginate or an alginate derivative, gelatin, collagen, agarose, hyaluronic acid, poly(lysine), polyhydroxybutyrate, poly-epsilon-caprolactone, polyphosphazines, poly(vinyl alcohol), poly(alkylene oxide), poly(ethylene oxide), poly(allylamine), poly(acrylate), poly(4-aminomethylstyrene), pluronic polyol, polyoxamer, poly(uronic acid), poly(anhydride), poly(vinylpyrrolidone), and any combination thereof. In some embodiments, the scaffold comprises pores having a diameter between about 1 μm and 100 μm.

In some embodiments, the C-terminus of the Notch ligand is conjugated to the selected support. In some embodiments, this can include adding a sequence at the C-terminal end of the Notch ligand that can be enzymatically conjugated to the support, for example, through a biotin molecule. In another embodiment, a Notch ligand-Fc fusion is prepared, such that the Fc segment can be immobilized by binding to protein A or protein G that is conjugated to the support. Of course, any of the known protein conjugation methods can be employed.

Thus, in various embodiments, the Notch ligand is immobilized, functionalized, and/or embedded in 2D or 3D culture system. The Notch ligand may be incorporated along with a component of extracellular matrix, such as one or more selected from fibronectin, RetroNectin, and laminin. In some embodiments, the Notch ligand and/or component of extracellular matrix are embedded in inert materials providing 3D culture conditions. Exemplary materials include, but are not limited to, cellulose, alginate, and combinations thereof. In some embodiments, the Notch ligand, a component of extracellular matrix, or combinations thereof, are in contact with culture conditions providing topographical patterns and/or textures (e.g., roughness) to cells conducive to differentiation and/or expansion.

In some embodiments, cell populations or banks are differentiated to progenitor T cells by culture in medium comprising TNF-α and/or antagonist of aryl hydrocarbon/dioxin receptor (SR1), and in the presence of Notch ligand. See US 2020/0390817, US 2021/0169934, and US 2021/0169935, which are hereby incorporated by reference in its entirety. In some embodiments the HSCs are cultured in a medium comprising TNF-α, IL-7, thrombopoietin (TPO), Flt3L, and stem cell factor (SCF), and optionally SR1, in the presence of an immobilized Delta-Like-4 ligand and a fibronectin fragment. In some embodiments, the cells are cultured with RetroNectin, which is a recombinant human fibronectin containing three functional domains: the human fibronectin cell-binding domain (C-domain), heparin-binding domain (H-domain), and CS-1 sequence domain. In some embodiments, cells are cultured in the presence of an immobilized Delta-Like-4 ligand and a RetroNectin. In some embodiments, cells are cultured in the presence of an immobilized Delta-Like-4 ligand, TNF-alpha, and a RetroNectin. In some embodiments, cells are cultured in the presence of an immobilized Delta-Like-1 ligand and a RetroNectin. In some embodiments, cells are cultured in the presence of SFIP3 and RetroNectin. In some embodiments, cells are cultured in the presence of an immobilized Delta-Like-4 ligand and SHH molecules and/or functional derivatives thereof. Exemplary fibronectin fragments include one or more RGDS, CS-1, and heparin-binding motifs. Fibronectin fragments can be free in solution or immobilized to the culture surface or on particles. In some embodiments, cells are cultured for 5 to 7 days to prepare CD7+ progenitor T cells.

In various embodiments, the method produces progenitor T cells, or a T cell lineage, by culturing the HSC population with the Notch ligand (including any of the embodiments described above) with or without component(s) extracellular matrix, and optionally adding TNF-alpha to the culture at certain stages of differentiation. Thus, cells created in some embodiments are progenitor or precursor cells committed to the T cell lineage (“progenitor T cells”). In some embodiments, the cells are CD7⁺ progenitor T cells. In some embodiments, the cells are CD25⁺ immature T cells, or cells that have undergone CD4 or CD8 lineage commitment. In some embodiments, the cells are CD4⁺CD8⁺ double positive (DP), CD4⁻CD8⁺, or CD4⁺CD8⁻. In some embodiments, the cells are single positive (SP) cells that are CD4⁻CD8⁺ or CD4⁺CD8⁻ and TCR^{hi}. In some embodiments, the cells are TCRαβ⁺ and/or TCRγΔ⁺. In various embodiments, the cells are CD3⁺.

The adoptive transfer of progenitor T cells is a strategy for enhancing T cell reconstitution. Progenitor T cells are developmentally immature and undergo positive and negative selection in the host thymus. Thus, they become restricted to the recipient's major histocompatibility complex (MHC) yielding host tolerant T cells that can bypass the clinical challenges associated with graft-versus-host disease (GVHD). Importantly, engraftment with progenitor T cells restores the thymic architecture and improves subsequent thymic seeding by HSC-derived progenitors. In addition to its intrinsic regenerative medicine properties, progenitor T cells can also be engineered with T cell receptors (TCRs) and chimeric antigen receptors (CARs) (via either gene or mRNA delivery) to confer specificity to tumor-associated antigens.

In various embodiments, the progenitor T cells are further cultured under suitable conditions to generate cells of a desired T cell lineage, including with one or more Notch ligands. For example, the cells can be cultured in the presence of one or more Notch ligands as described for a sufficient time to form cells of the T cell lineage. In some embodiments, stem cells or progenitor T cells are cultured in suspension with soluble Notch ligand or Notch ligand conjugated to particles or other supports, or Notch ligand expressing cells. In some embodiments, the progenitor T cells or stem cells are cultured in suspension or in adherent format in a bioreactor, optionally a closed or a closed, automated bioreactor, with a soluble or conjugated Notch ligand in suspension. One or more cytokines, extracellular matrix component(s), and thymic niche factor(s) that promote commitment and differentiation to the desired T cell lineage may also be added to the culture or reactor. Such cytokines or factors are known in the art. In various embodiments, the HSC population is cultured with the Notch ligand for about 4 to about 21 days, or from about 6 to about 18 days, or from about 7 to about 14 days to generate progenitor T cells. In some embodiments, the stem cell population or derivative thereof is cultured for at least about 21 days or at least about 28 days to generate mature T cell lineages or NK cells.

In various embodiments, the cell populations such as an HSC population is cultured in an artificial thymic organoid (ATO). See, Hagen, M. et al. (2019). The ATO will include culture of HSCs (or aggregates of HSCs) with a Notch ligand-expressing stromal cell line in serum-free conditions. The artificial thymic organoid is a 3D system, inducing differentiation of hematopoietic precursors to naive CD3⁺CD8⁺ and CD3⁺CD4⁺ T cells.

In various embodiments, the method comprises generating a derivative of the progenitor T cells or generating a T cell lineage from the progenitor T cells. In certain embodiments, the derivative of the progenitor T cell or T cell lineage expresses CD3 and a T cell receptor. In some embodiments, the T cell lineage is CD8⁺ and/or CD4⁺. For example, T cells lineages can include one or more of CD8⁺CD4⁻, CD8⁻CD4⁺, CD8⁺CD4⁺, and CD8⁻CD4⁻ cells. In some embodiments, the iPSCs, CD34+ cells, or derivatives thereof are modified to express a chimeric antigen receptor (CAR) at progenitor-T, T-cell, and/or NK cell level.

In some embodiments, the T cell lineage is a regulatory T cell. T regulatory cells (or T regs) are defined as CD4⁺CD25⁺. Tregs control the immune response to self and foreign antigens and help prevent autoimmune disease. Differentiation of progenitor T cells to Tregs in some embodiments involves ectopic expression of FOXP3 and culturing the progenitor T cells or Treg precursors with one or more growth factors, such as but not limited to IL-2.

In some embodiments, the cell populations or banks of expanded primary cells, or derivatives of iPSCs (e.g., HSC population) are differentiated to B lymphocytes (“B cells”). For example, culturing CD34+ or CD34+CD43+ cells with MS5 stromal cells or S17 stromal cells (e.g., for 15-25 days, or about 21 days) can generate a B-lymphoid identity with expression of CD19, CD45, and CD10. See Carpenter L. et al., Human induced pluripotent stem cells are capable of B-cell lymphopoiesis, Blood 117 (15): 4008-4011. Dubois F. et al., Toward a better definition of hematopoietic progenitors suitable for B cell differentiation, Plos One Dec. 15, 2020. In various embodiments, the B cells produced according to this disclosure express surface IgM (sIgM) and undergo VDJ rearrangement. In various embodiments, B cells produced according to this disclosure will engraft in the spleen and secondary lymphoid tissues of a subject for maturation.

In some embodiments, the cell populations are differentiated to monocytes, macrophages, or neutrophils. For example, erythromyeloid precursors (EMP) (CD43+CD45+) may be generated by culture with IL-6, IL-3, thyroid peroxidase (TPO), SCF, FGF2, and VEGF, followed by differentiation to monocytes. Differentiation to monocytes to employ culture with M-CSF, IL-3, and IL-6. See Cao X et al., Differentiation and Functional Comparison of Monocytes and Macrophages from hiPSCs with Peripheral Blood Derivatives, Stem Cell Reports. 2019 Jun. 11; 12 (6): 1282-1297. Monocytes and macrophage lineages prepared according to this disclosure are CD14+ and will exhibit endocytosis and phagocytic functions. In some embodiments, macrophages are polarized ex vivo to the M1 (pro-inflammatory) or M2 (immunosuppressive) phenotype. In some embodiments, CD45+ hematopoietic cells with phagocytic markers, such as CD33 and CD11b, are generated, and optionally subsequently to cells with neutrophil specific markers, such as CD66b, CD16b, GPI-80, etc., by differentiation of iPSC derived hCD34+ cells.

These processes can employ differentiation media containing mixtures of cytokines and growth factors, including but not limited to SCF, IL3, FLT3, IL6, GM-CSF, G-CSF, EPO, TPO, and/or combinations thereof. In some embodiments, neutrophils and their precursors are generated by methods described in: Saeki L., et al., A Feeder-Free and Efficient Production of Functional Neutrophils from Human Embryonic Stem Cells, Stem Cells Vol. 27, Issue 1, 2009, Pages 59-67; Morishima T. et al., Neutrophil differentiation from human-induced pluripotent stem cells. J. Cell. Physiol. 226:1283-1291, 2011; Yokoyama Y. et al., Derivation of functional mature neutrophils from human embryonic stem cells. Blood 2009 Jun. 25; 113 (26): 6584-92; and Sweeney C L et al., Generation of functionally mature neutrophils from induced pluripotent stem cells. Methods Mol Biol 2014; 1124:189-206.

In some embodiments, the HSC population or fraction thereof are differentiated to megakaryocytes or platelets. For example, megakaryocytes (as a renewable source for platelets) can be prepared from the HSCs or fraction thereof by culture with SCF, IL-11, and TPO for several days (e.g., about 5 days). Alternatively, other cytokines and growth factors such as IL-3, IL-6, SDF-1, and FGF-4 can be employed. Megakaryocytes will be CD42b+CD61+. See Liu L., Efficient Generation of Megakaryocytes From Human Induced Pluripotent Stem Cells Using Food and Drug Administration-Approved Pharmacological Reagents, Stem Cells Transl Med. 2015 April; 4 (4): 309-319. Platelets can be further generated from megakaryocytes by culture in serum free media with II-11. CD41+CD42a+ platelet-like-particles are recovered from the media.

In some embodiments, the derivative of the progenitor T cell is a natural killer (NK) cell. In some embodiments, NK cells are generated from progenitor T cells as described in U.S. Pat. No. 10,266,805, which is hereby incorporated by reference in its entirety. For example, the progenitor T cells can give rise to NK cells when cultured with IL-15. In some embodiments, the NK cell expresses a CAR, based on gene editing of iPSC, embryonic bodies, hCD34+ cells, or NK cells, or via mRNA expression in NK cells.

In some embodiments, the HSC population or fraction thereof is differentiated to red cells or derivatives thereof. Red cells produced according to this disclosure can be administered or used in therapy, for example, for an inherited or acquired red cell disorder, bone marrow failure disorder, high-altitude-related physiological and pathological condition, conditions related to chemicals or radiation exposure, and/or for treatment of subjects undergoing HSC transplant. In further embodiments, the red cells prepared according to this disclosure are provided as a pharmaceutical acceptable composition delivering or encapsulating drugs (including but not limited to enzymes), oxygen carriers, or other suitable materials to treat human disease or physiological or pathological conditions.

In other aspects, the disclosure provides a cell population, or pharmaceutically acceptable composition thereof, according to this disclosure. In some embodiments, the cell population is a lymphocyte population capable of engraftment in a thymus, spleen, or secondary lymphoid organ upon administration to a subject in need. In various embodiments, the composition for cellular therapy is prepared that comprises the desired cell population a pharmaceutically acceptable vehicle.

In some embodiments, the cell composition comprises an iPSC or HSC cell population (or population differentiated therefrom) that is HLA-A^{neg}, HLA-DPB1^{neg}, and HLA-DQB100g. Despite such gene deletions and/or gene edits, the iPSCs or HSCs and cells derived (e.g., differentiated therefrom) retain full antigen presenting functionality and ability to differentiate from precursors to hematopoietic lineages (as described herein). Cell compositions of this aspect provide advantages in HLA matching for a recipient, to avoid, for example, GVHD. In various embodiments, the population is homozygous for both HLA-B and HLA-C. In some embodiments, the population is homozygous for HLA-DRB1. In embodiments, the cells of the composition have an HLA haplotype as described herein.

In various embodiments, the cells of the HSC composition are at least about 50% CD34+, or at least about 60% CD34+, or at least about 75% CD34+, or at least about 80% CD34+, or at least about 85% CD34+, or at least about 90% CD34+, or at least about 95% CD34+. In addition, in embodiments, at least about 50%, or at least about 60%, or at least about 75%, or at least about 80%, or at least about 85%, or at least about 90%, or at least about 95% cells in the composition are one or more of CD90+ and CD45+.

The pharmaceutical composition may comprise at least about 10² cells, or at least about 10³, or at least about 10⁴, or at least about 10⁵, or at least about 10⁶, or at least about 10⁷, or at least about 10⁸ cells, or at least about 10⁹ cells, or at least about 10¹⁰ cells, or at least about 10¹¹ cells, or at least about 10¹² cells, or at least about 10¹³ cells, or at least about 10¹⁴ cells. For example, in some embodiments, the pharmaceutical composition is administered, comprising HSCs of from about 100,000 to about 400,000 cells per kilogram of recipient body weight (e.g., about 200,000 cells/kg). In other embodiments, cells are administered at from about 10⁵ to about 5×10⁵ cells per kilogram (e.g., about 2.5×10⁵ cells/kg), or from about 106 to about 5×10⁶ cells per kilogram (e.g., about 2.5×10⁶ cells/kg), or from about 5×10⁶ to about 10⁷ cells per kilogram (e.g., about 5×10⁶ cells/kg) or from about 107 to about 10⁸ cells per kilogram (e.g., about 5×10⁷ cells/kg) or from about 108 to about 10⁹ cells per kilogram (e.g., about 5×10⁸ cells/kg) or from about 10⁹ to about 10¹⁰ cells per kilogram or from about 10¹⁰ to about 10¹¹ cells or from about 10¹¹ to about 10¹² cells per kilogram or from about 10¹² to about 10¹³ cells per kilogram or from about 10¹³ to about 10¹⁴ cells per kilogram of a recipient's body weight.

The cell composition of this disclosure may further comprise a pharmaceutically acceptable carrier or vehicle suitable for intravenous infusion or other administration route, and the composition may include a suitable cryoprotectant. An exemplary carrier is DMSO (e.g., about 10% DMSO). Cell compositions may be provided in unit vials or bags and stored frozen until use. In certain embodiments, the volume of the composition is from about one fluid ounce to one pint.

In some embodiments, this disclosure provides a CD7+ progenitor T cell, or pharmaceutically acceptable composition thereof, where the CD7+ progenitor T cell produced by a method disclosed herein. In various embodiments, the progenitor T cell is capable of engraftment in a thymus or spleen of a recipient. Progenitor T cells have the potential to decrease the risk of relapse of leukemia or other types of cancer in bone marrow transplant patients and to decrease the number of infections post-transplant that cause significant morbidity and mortality in patients. In another aspect, this disclosure provides a derivative of the progenitor T cell or T cell lineage produced by a method disclosed herein, or a pharmaceutically acceptable composition thereof.

In some embodiments, the cell population is a T cell population (or progenitor T cell population) or NK cell population, which are useful for adoptive cell therapy, for example, for human subjects having a condition selected from lymphopenia, a cancer, an immune deficiency, a viral infection, an autoimmune disease (particularly where the T cell population comprises Tregs), a skeletal dysplasia, a bone marrow failure syndrome, or a genetic disorder that impairs T cell development or function. Exemplary genetic disorders can impact the immune system, manifesting as an immunocompromised state, or autoimmune or pro-inflammatory state. In some embodiments, the subject has cancer, which is optionally a hematological malignancy or a solid tumor. In some embodiments, the T cell is a CAR-T cell.

In some embodiments, the cell population is a B lymphocyte population, and is capable of engraftment in a spleen or secondary lymphoid tissue of a subject. B-cell populations according to this disclosure have the potential to partially reconstitute humoral immunity in an immune compromised patient, for example, providing protection from or treatment for infectious diseases, including viral, bacterial, fungal, or parasite infection. In various embodiments, the B cells according to this disclosure are capable of differentiation to plasma cells for production of antigen-specific antibodies in vivo. In other embodiments, B cells produced according to this disclosure can be employed for cancer immunotherapy. In some embodiments, chimeric antigen B cells (CAR B cells) are prepared by gene modifications at iPSC, embryonic bodies, hCD34+ cells, hematopoietic progenitor cell, or B cell level. CAR B cells express a surface BCR and/or secrete a recombinant monoclonal antibody that recognizes a target antigen, such as a cancer antigen or an infectious disease antigen. In still other embodiments, B cells produced according to this disclosure are used for ex vivo production of antibodies (e.g., vaccine antibodies for providing protection from an infectious agent).

In some embodiments, the cell population is a monocyte or macrophage cell population, and the cell population is capable of engraftment and maturation in various tissues of a subject, including tumors. In various embodiments, the monocyte or macrophage cell population is able to form tissue resident macrophages in a subject. In various embodiments, the macrophages are predominately of the M1 (pro-inflammatory) or M2 (immunosuppressive) phenotype. In various embodiments, the subject in need to treatment has a cancer of any of various tissues or organs, liver or kidney inflammatory disease, or bacterial infection (e.g., sepsis or infection or colonization of an indwelling medical device).

In some embodiments, the cell population is a megakaryocyte population, or is platelets developed therefrom. These cells or platelets are useful for treating inherited platelet defects, impacting for example, coagulation pathways.

In some embodiments, the cell population is a red cell population.

In still other embodiments, the iPSCs are differentiated to non-hematopoietic stem cells or precursor cells, or cells or tissues differentiated therefrom. Such cells include mesenchymal stem cells, neural stem cells, epithelial stem cells, neuronal cells (or precursors thereof) (including cortical, dopaminergic, and motor neurons, or precursors thereof), astrocytes (or precursors thereof), oligodendrocytes (or precursors thereof), cardiomyocytes (or precursors thereof), skeletal muscle cells (or precursors thereof), hepatocytes (or precursors thereof), pancreatic β cells (or precursors thereof), and lung epithelial cells (or precursors thereof).

The cell composition of this disclosure may further comprise a pharmaceutically acceptable excipient or a carrier. Such excipients or carrier solutions also can contain buffers, diluents, and other suitable additives. A buffer refers to a solution or liquid whose chemical makeup neutralizes acids or bases without a significant change in pH. Examples of buffers envisioned by the disclosure include, but are not limited to, normal/physiologic saline (0.9% NaCl), 5% dextrose in water (D5W), Dulbecco's phosphate buffered saline (PBS), Ringer's solution. Pharmaceutically acceptable carrier can be selected from, but not limited to, a filler (e.g., polyacrylates, microcrystalline cellulose, pectin, gelatin, calcium sulfate, ethyl cellulose, lactose and other sugars, calcium hydrogen phosphate, etc.), a binding agent (e.g., polyvinylpyrrolidone, pregelatinized maize starch or hydroxypropyl methylcellulose, etc.), a disintegrant (e.g., sodium starch glycolate, starch, etc.), or a wetting agent (e.g., sodium lauryl sulfate (SDS), etc.). a lubricant (e.g., talc, silica, magnesium stearate, stearic acid, colloidal silicon dioxide, metallic stearates, corn starch, hydrogenated vegetable oils, polyethylene glycols, sodium benzoate, sodium acetate, etc.). Other suitable pharmaceutically acceptable carriers for the compositions of the present disclosure include, but are not limited to, alcohols, water, silicic acids, salt solutions, gelatins, polyethylene glycols, amyloses, magnesium stearates, talc, hydroxy methylcelluloses, viscous paraffins, polyvinylpyrrolidones and the like. Further it may comprise a vehicle suitable for intravenous infusion or other administration route (intra-arterially or intravenous injection, subcutaneous injection, with collagen sponge seeding with fibril glue seeding, with nanoparticles), and the composition may include a suitable cryoprotectant. An exemplary carrier is DMSO (e.g., about 10% DMSO) Other carriers may include dimethoxy ethane (DME), N,N-dimethylformamide (DMF), or dimethylacetamide, including mixtures or combinations thereof. Cell compositions may be provided in implantable devices (e.g., scaffolds) or in bags or in vials, tubes or a container in an appropriate volume and stored frozen until use.

The pharmaceutical compositions for use in the disclosed methods may also contain additional therapeutic agents for treatment of the particular targeted disorder. For example, a pharmaceutical composition may also include cytokines and growth factors (interleukins, interferons, FGF, VEGF, PDGF, PIGF, STAT etc.). Such additional factors and/or agents may be included in the pharmaceutical composition to produce advantages of the therapeutic approaches disclosed herein, i.e., provide improved therapeutic efficacy with reduced systemic toxicity.

Routes of administration of the derivatives of cell line(s) or banks of expanded primary cells or derivatives of iPSC cells or banks thereof (e.g., HSCs or progenies thereof) could be by any suitable means, including but not limited to, parenteral, subcutaneous, intraperitoneal, intrapulmonary, and intranasal, and, if desired for local immunosuppressive treatment, intralesional administration. Parenteral infusions include intramuscular, intravenous, intraarterial, intraperitoneal, or subcutaneous administration. In addition, the cells (e.g., HLA modified iPSCs or progenitors or progenies thereof) may suitably be administered by pulse infusion, e.g., with declining doses of the cells (e.g., HLA modified iPSCs or HSCs and progenitors or progenies thereof). In some embodiments the dosing is given by injections, for example via intravenous or subcutaneous injections, depending in part on whether the administration is brief or chronic.

The derivatives of cell line(s) or banks of expanded primary cells (e.g., HSCs and progenies thereof) can be formulated, dosed, and administered in a fashion consistent with good medical practice. Factors for consideration in this context include the particular disease or disorder being treated, the particular mammal being treated (e.g., human), the clinical condition of the individual patient, the cause of the disease or disorder, the site of delivery of the agent, the method of administration, the scheduling of administration, and other factors known to medical practitioners. The therapeutically effective amount of the cells (HSCs or progenitors or progenies derived from the cell line(s) or banks of expanded primary cells or derivatives of iPSC cells or banks thereof) to be administered will be governed by such considerations.

Administration may be in a single dose or may be multiple doses delivered in intervals of 3 to 4 hours, 1-4 times a day, 1-4 times a week; 1-4 times a month, 1-7 times a month, or administration occurs once every 3 or 4 weeks for several months.

One may administer other compounds, such as cytotoxic agents, immunosuppressive agents and/or cytokines or growth factors (e.g., stem cell factor, thrombopoietin, transforming growth factor (TGF)-α or β, fibroblast growth factors (FGF), angiopoietin (Ang) family of growth factors, insulin-like growth factors, granulocyte-macrophage colony-stimulating factor, TNF-α or β, VEGF, interleukins (e.g., IL-2, 6, 7, 8 10, 12, 15 etc.,) and interferons (e.g., INF-alpha or gamma)) with the cells (e.g., HLA modified iPSC-derived HSCs or progenitors or progenies thereof) herein. The combined administration includes co-administration, using separate formulations or a single pharmaceutical formulation, and consecutive administration in either order, wherein preferably there is a time period while both (and all) active agents simultaneously exert their biological activities.

Therapeutic formulations of the cells (e.g., derivatives of cell line(s) or banks of expanded primary cells, such as HSCs and progenies thereof) used in accordance with the present disclosure are prepared for storage by mixing cells (e.g., iPSC-derived HSCs or progenitors or progenies thereof) having the desired degree of purity with optional pharmaceutically acceptable carriers, excipients or stabilizers, such acceptable carriers, excipients or stabilizers are disclosed in Remington: The Science and Practice of Pharmacy, Twenty Third Edition: Elsevier (2020), incorporated herein in its entirety by reference, in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyl dimethyl benzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrin; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG), for example, a PEG chain having a molecular weight between 1,000-15,000 Daltons, or between 2,000 and 10,000 Daltons, or between 2,000 and 5,000 Daltons. Other hydrophilic polymers which may be suitable include polyvinylpyrrolidone, polymethyloxazoline, polyethyloxazoline, polyhydroxypropyl methacrylamide, polymethacrylamide and polydimethylacrylamide, polylactic acid, polyglycolic acid, and derivatized celluloses, such as hydroxymethylcellulose or hydroxyethylcellulose.

Lyophilized formulations adapted for subcutaneous administration are also contemplated by the disclosure. Such lyophilized formulations may be reconstituted with a suitable diluent to an optimal concentration and the reconstituted formulation may be administered subcutaneously to the mammal to be treated herein.

The formulation herein may also contain more than one active agent as necessary for the particular indication being treated, preferably those with complementary activities that do not adversely affect each other. For example, it may be desirable to further provide a cytotoxic agent, cytokine, or immunosuppressive agent. The effective amount of such other agents depends on the amount of acceptable carriers, excipients, or stabilizers, present in the formulation, the type of disease or disorder or treatment, and other factors discussed above. These are generally used in the same dosages and with administration routes as used hereinbefore or about from 1 to 99% of the heretofore employed dosages.

Agents such as hormones, growth factors and cytokines antibodies that may be co-administered with the derivatives of cell line(s) or banks of expanded primary cells (e.g., iPSC-derived HSCs or progenitors or progenies thereof) of the present disclosure includes molecules such as renin; a growth hormone, including human growth hormone and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor vmc, factor IX, tissue factor (TF), and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; RANTES (regulated on activation normally T-cell expressed and secreted); human macrophage inflammatory protein (MIP-1-alpha); a serum albumin, such as human serum albumin; Muellerian-inhibiting substance; relaxin A-chain; relaxin B-chain; prorelaxin; mouse gonadotropin-associated peptide; a microbial protein, such as beta-lactamase; DNase; IgE; a cytotoxic T-lymphocyte associated antigen (CTLA), such as CTLA-4; inhibin; activin; vascular endothelial growth factor (VEGF); receptors for hormones or growth factors; protein A or D; rheumatoid factors; a neurotrophic factor such as bone-derived neurotrophic factor (BDNF), neurotrophin-3, -4, -5, or -6 (NT-3, NT4, NT-5, or NT-6), or a nerve growth factor such as NGF-beta; platelet-derived growth factor (PDGF); fibroblast growth factor such as aFGF and bFGF; fibroblast growth factor receptor 2 (FGFR2), epidermal growth factor (EGF); transforming growth factor (TGF) such as TGF-alpha and TGF-beta, including TGF-beta1, TGF-beta2, TGF-beta3, TGF-beta4, or TGF-beta5; bone morphogenetic protein (BMP), including BMP1, BMP6, BMP7, and BMP-receptor 2; insulin-like growth factor-I and -II (IGF-I and IGF-II); des (1-3)-IGF-I (brain IGF-I), insulin-like growth factor binding proteins, hepatocyte growth factor (HGF), EpCAM, GD3, FLT3, PSMA, PSCA, MUCI, MUC16, STEAP, CEA, TENB2, EphA receptors, EphB receptors, folate receptor, FOLRI, mesothelin, cripto, alphavbeta6, integrins, VEGF, VEGFR, EGFR, tarnsferrin receptor, IRTA1, IRTA2, IRTA3, IRTA4, IRTA5; CD proteins such as CD2, CD3, CD4, CD5, CD6, CD8, CD11, CD14, CD19, CD20, CD21, CD22, CD25, CD26, CD28, CD30, CD33, CD36, CD37, CD38, CD40, CD44, CD52, CD55, CD56, CD59, CD70, CD79, CD80. CD81, CD103, CD105, CD134, CD137, CD138, CD152, TNF alpha, IFN alpha, GM-CSF, IL-3 or an antibody which binds to one or more tumor-associated antigens or cell-surface receptors; erythropoietin; osteoinductive factors; immunotoxins; a bone morphogenetic protein (BMP); an interferon, such as interferon-alpha,-beta, and -gamma; colony stimulating factors (CSFs), e.g., M-CSF, GM-CSF, and G-CSF; interleukins (ILs), e.g., IL-2, IL-6, IL-12, IL-23, IL-12/23 p40, IL-17, IL-15, IL-21, IL-1a, IL-1b, IL-18, IL-8, IL-4, IL-3, and IL-5; superoxide dismutase; T-cell receptors; surface membrane proteins; decay accelerating factor; viral antigen such as, for example, a portion of the HIV envelope; transport proteins; homing receptors; addressins; regulatory proteins; integrins, such as CD11a, CD11b, CD11c, CD18, an ICAM, VLA-4 and VCAM; a tumor associated antigen such as HER2, HER3 or HER4 receptor; endoglin, c-Met, c-kit, 1GF1R, PSGR, NGEP, PSMA, PSCA, LGR5, B7H4, TAG72 (tumor-associated glycoprotein 72) and fragments of any of the above-listed polypeptides.

Examples of antibodies or fragments thereof that may be administered include, but are not limited to, anti-PD-L1 antibodies, abciximab (Reopro), adalimumab (Humira, Amjevita), alefacept (Amevive), alemtuzumab (Campath), basiliximab (Simulect), belimumab (Benlysta), bezlotoxumab (Zinplava), canakinumab (Ilaris), certolizumab pegol (Cimzia), cetuximab (Erbitux), daclizumab (Zenapax, Zinbryta), denosumab (Prolia, Xgeva), efalizumab (Raptiva), golimumab (Simponi, Simponi Aria), inflectra (Remicade), ipilimumab (Yervoy), ixekizumab (Taltz), natalizumab (Tysabri), nivolumab (Opdivo), olaratumab (Lartruvo), omalizumab (Xolair), palivizumab (Synagis), panitumumab (Vectibix), pembrolizumab (Keytruda), rituximab (Rituxan), tocilizumab (Actemra), trastuzumab (Herceptin), secukinumab (Cosentyx), ranibizumab, abciximab, raxibacumab, caplacizumab, infliximab, bevacizumab, dabigatran, Idarucizumab, or ustekinumab (Stelara) or a combination thereof. Furthermore, the antibodies may be selected from anti-estrogen receptor antibody, anti-progesterone receptor antibody, anti-p53 antibody, anti-EGFR antibody, anti-cathepsin D antibody, anti-Bcl-2 antibody, anti-E-cadherin antibody, anti-CA125 antibody, anti-CA15-3 antibody, anti-CA19-9 antibody, anti-c-erbB-2 antibody, anti-P-glycoprotein antibody, anti-CEA antibody, anti-retinoblastoma protein antibody, anti-ras oncoprotein antibody, anti-Lewis X antibody, anti-Ki-67 antibody, anti-PCNA antibody, anti-CD3 antibody, anti-CD4 antibody, anti-CD5 antibody, anti-CD7 antibody, anti-CD8 antibody, anti-CD9/p24 antibody, anti-CD1-antibody, anti-CD11c antibody, anti-CD13 antibody, anti-CD14 antibody, anti-CD15 antibody, anti-CD19 antibody, anti-CD20 antibody, anti-CD22 antibody, anti-CD23 antibody, anti-CD30 antibody, anti-CD31 antibody, anti-CD33 antibody, anti-CD34 antibody, anti-CD35 antibody, anti-CD38 antibody, anti-CD39 antibody, anti-CD41 antibody, anti-LCA/CD45 antibody, anti-CD45RO antibody, anti-CD45RA antibody, anti-CD71 antibody, anti-CD95/Fas antibody, anti-CD99 antibody, anti-CD100 antibody, anti-S-100 antibody, anti-CD106 antibody, anti-ubiquitin antibody, anti-c-myc antibody, anti-cytokeratin antibody, anti-lambda light chains antibody, anti-melanosomes antibody, anti-prostate specific antigen antibody, anti-tau antigen antibody, anti-fibrin antibody, anti-keratins antibody, and anti-Tn-antigen antibody.

Co-administration does not require the therapeutic agents to be administered simultaneously, if the timing of their administration is such that the pharmacological activities of the additional therapeutic agent and the active ingredient(s) in the pharmaceutical composition overlap in time, thereby exerting a combined therapeutic effect. In general, each agent will be administered at a dose and on a time, schedule determined for that agent.

The present pharmaceutical compositions may be administered in any dose appropriate to achieve a desired outcome. In some embodiments, the desired outcome is a reduction in the intensity, severity, frequency, and/or delay of onset of one or more symptoms of infection. In some embodiments, the desired outcome is the inhibition or prevention of infection. The dose required will vary from subject to subject depending on the species, age, weight, and general condition of the subject, the severity of the infection being prevented or treated, the particular composition being used, and its mode of administration.

In some embodiments, pharmaceutical compositions in accordance with the present disclosure are administered in single or multiple doses. In some embodiments, the pharmaceutical compositions are administered in multiple doses administered on different days.

The active ingredients (e.g., HSCs or progenitors or progenies thereof derived from the cell line(s) or banks of expanded primary cells or derivatives of iPSC cells or banks thereof) may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions. Such techniques are well known to one of skill in the art and are disclosed in Remington: The Science and Practice of Pharmacy, Twenty Third Edition: Elsevier (2020).

Sustained-release preparations may be prepared. Suitable examples of sustained-release preparations include semi permeable matrices of solid hydrophobic polymers containing the HSCs, progenitors, or progenies thereof, which matrices are in the form of shaped articles, e.g., films, or microcapsules. Examples of sustained-release matrices include hydrogels (for example, poly(2-hydroxyethyl-methacrylate), or polyesters, poly(vinylalcohol)), polylactides (U.S. Pat. No. 3,773,919), copolymers of L-glutamic acid and gamma ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyricacid.

The formulations to be used for in vivo administration must be sterile. This is readily accomplished by filtration through sterile filtration membranes and other techniques known to one of skill in the art.

As used herein, unless the context requires otherwise, the term “about” means ±10% of the associated numerical value.

Certain aspects and embodiments of this disclosure are further described with reference to the following examples.

### EXAMPLES

### Example 1: ETV2 Over-Expression Increases the Yield of Hemogenic Endothelial Cells and Enhances the CD34+ Cell Formulation During iPSC Differentiation but does not Affect Pluripotency

### Methods

iPSCs were developed from hCD34+ cells by episomal reprogramming as known in the art and essentially as described in Yu, et al. Induced pluripotent stem cell lines derived from human somatic cells, Science 318, 1917-1920, (2007); and J. Yu, et al. Human induced pluripotent stem cells free of vector and transgene sequences. Science 324, 797-801, (2009). Embryoid Bodies and hemogenic endothelium differentiation was performed essentially as described in: R. Sugimura, et al., Haematopoietic stem and progenitor cells from human pluripotent stem cells. Nature 545, 432-438, (2017); C. M. Sturgeon, et al, Wnt signaling controls the specification of definitive and primitive hematopoiesis from human pluripotent stem cells. Nat Biotechnol 32, 554-561, (2014); J. Yu, et al. Induced pluripotent stem cell lines derived from human somatic cells. Science 318, 1917-1920, (2007); and J. Yu, et al. Human induced pluripotent stem cells free of vector and transgene sequences. Science 324, 797-801, (2009).

Briefly, hiPSC were dissociated and resuspended in media supplemented with L-glutamine, penicillin/streptomycin, ascorbic acid, human holo-Transferrin, monothioglycerol, BMP4, and Y-27632. Next, cells were seeded in 10 cm dishes (EZSPHERE or low attachment plate) for the EB formation. On Day 1, bFGF and BMP4 were added to the medium. On Day 2, the media was replaced with a media containing SB431542, CHIR99021, bFGF, and BMP4. On Day 4, the cell media was replaced with a media supplemented with VEGF and bFGF. On day 6, the cell media was replaced with a media supplemented with bFGF, VEGF, IL-6, IGF-1, IL-11, SCF, and EPO. Cells were maintained in a 5% CO₂, 5% O₂, and 95% humidity incubator. To harvest the CD34+ cells, the EBs were dissociated on day 8, cells were filtered through a 70 μm strainer, and CD34+ cells were isolated by CD34 magnetic bead staining.

### Results

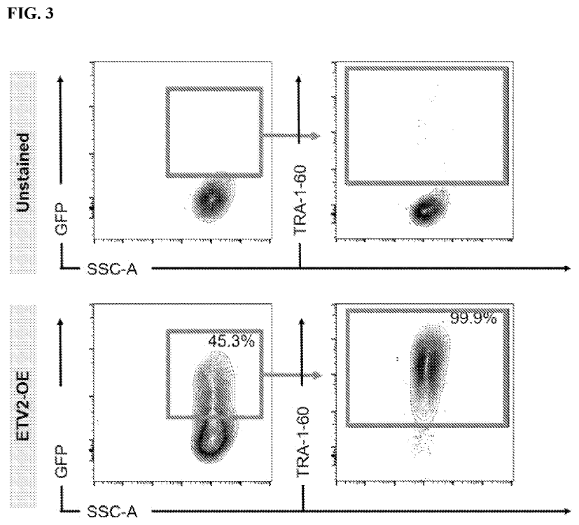

An adenoviral vector containing both ETV2 and GFP sequences under the control of the EF1A promoter was used to transduce induced pluripotent stem cells (iPSCs). After the transduction, about 45% of the iPSC culture was observed to be GFP positive, thus confirming ETV2 overexpression (ETV2-OE). It was further observed that ETV2-OE in iPSC cells preserves the pluripotency properties of iPSCs as shown by the stemness marker expression TRA-1-60 (FIG. 3). FIG. 3 shows FACS plots representative of transduction efficiency of iPSC with an adenoviral vector to overexpress the ETV2 and the GFP sequences.

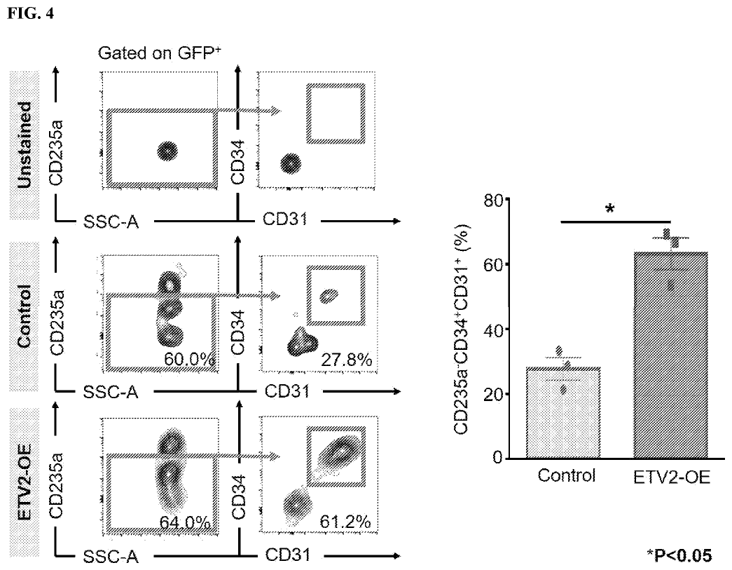

Next, the ETV2-OE-iPSCs were differentiated (along with control iPSCs transduced with a vector bearing the GFP sequence without ETV2) to embryoid bodies and subsequently to hemogenic endothelial cells (Strugeon et al., 2014). The results suggest that the overexpression of ETV2 boosts the formation of hemogenic endothelial cells as demonstrated by the expression of the CD34⁺ and CD31⁺markers within the CD235a⁻ population (FIG. 4). Specifically, FIG. 4 shows representative flow cytometric analysis of hemogenic endothelial cells (defined here as CD235a-CD34+CD31+) and relative quantification demonstrates that ETV2-OE enhances the formation of hemogenic endothelial cells as compared to controls.

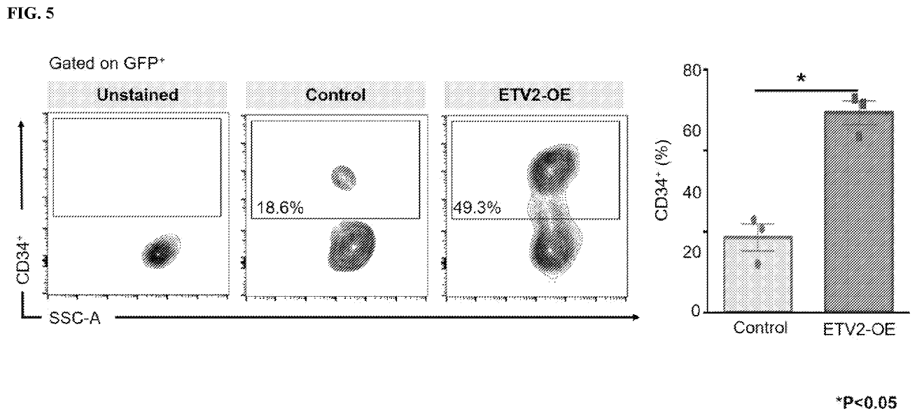

Moreover, the results suggest that ETV2-OE enhances the formation of the CD34″ cells (FIG. 5). FIG. 5 shows representative flow cytometric analysis of CD34+ cells and relative quantification demonstrates that ETV2-OE enhances the CD34+ cell formation.

Overall, these data indicate that ETV2 overexpression in iPSCs does not affect their pluripotency properties and facilitates their ability to undergo the hemogenic endothelial and hematopoietic differentiations.

### Example 2: IPSC-Derived HSCs Generated with Piezol Activation Undergo T Cell Differentiation Similar to Bone Marrow-Derived HSCs

### Methods

To analyze the EHT, EB-derived CD34+ cells were suspended in medium containing Y-27632, TPO, IL-3, SCF, IL-6, IL-11, IGF-1, VEGF, bFGF, BMP4, and FLT3. After the cells had adhered to the bottom of the wells for approximately 4-18 hours (by visual inspection), Yoda1 was added to the cultures for some experiments. After 4-7 days, the cells were collected for analysis.

iPSCs were differentiated to embryoid bodies for 8 days. At day 8, CD34+ cells from iPSC-derived embryoid bodies were harvested and cultured for additional 5 to 7 days to induce endothelial-to-hematopoietic (EHT) transition (with and without Yoda1). Then, CD34+ cells were harvested from the EHT culture between day 5 to day 7 for further hematopoietic lineage differentiation.

CD34+ cells, harvested from the EHT culture between day 5-7 (or total of day 13-21 differentiation from iPSCs), were seeded in 48-well plates pre-coated with rhDL4 and RetroNectin. T lineage differentiation was induced in media containing aMEM, FBS, ITS-G, 2BME, ascorbic acid-2-phosphate, Glutamax, rhSCF, rhTPO, rhIL7, FLT3L, rhSDF-1a, and SB203580.

Between day 2 to day 6, 80% of the media was changed every other day. At D7, cells were transferred into new coated plates and analyzed for the presence of pro-T cells (CD34+CD7+CD5+/−).

Between day 8 to day 13, 80% of the media was changed every other day. At D14, 100,000 cells/wells were transferred to a new coated plate and the cells analyzed for the presence of pre-T cells (CD34-CD7+CD5+/−).

Between day 15 to day 20, 80% of the media was changed every other day. Cells were harvested at D21, and the cells were analyzed for CD3, CD4, CD8, CD5, CD7, TCRab expression, as surrogates for T cells, via FACS, and/or activated using CD3/CD28 beads to evaluate their functional properties.

After 21 days of differentiation, cells were collected and re-seeded at approximately 80,000 cells into new 96-well culture plates in RPMI 1640 (no L-glutamine; no phenol red) plus FBS, L-glutamine, IL-2, and then activated with 1:1 CD3/CD28 beads. After 72 hours of activation with CD3/CD28 beads, cells were analyzed for CD3, CD69, CD25 expression by FACS and IFN-γ expression using RT-qPCR. The supernatant was analyzed by ELISA.

### Results

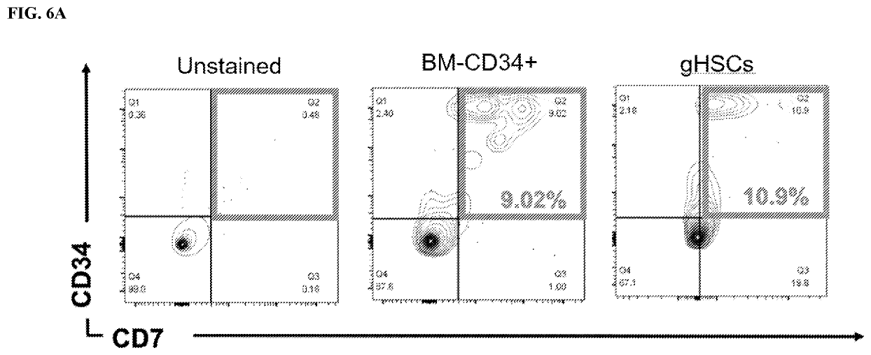

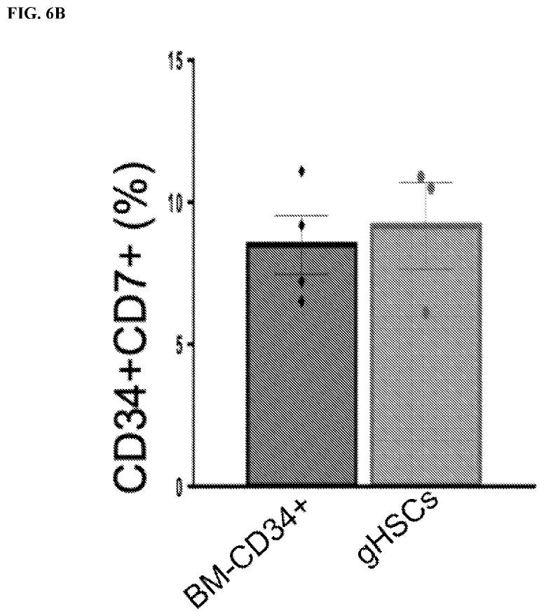

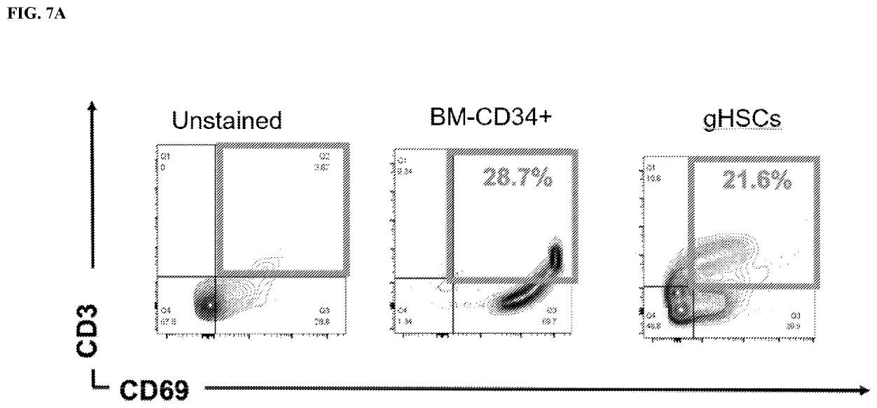

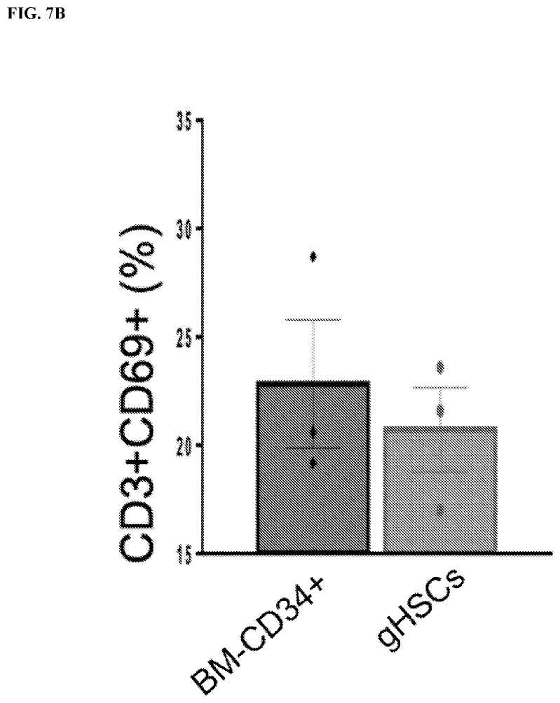

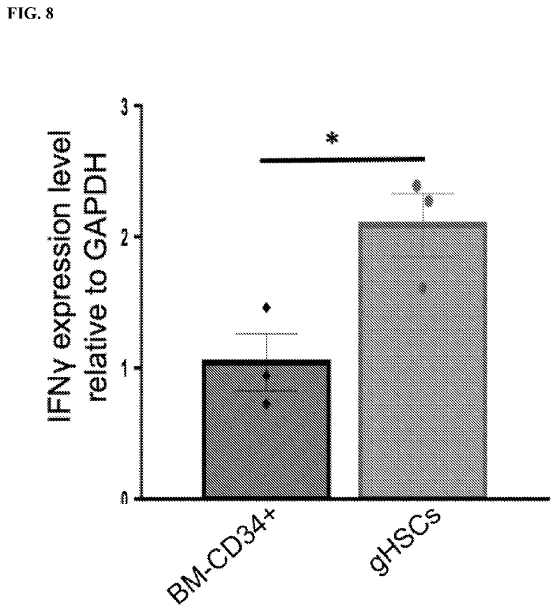

FIG. 6A and FIG. 6B show that iPSC-derived HSCs that are derived with EHT of CD34+ cells from differentiated iPSCs (e.g., in this case including Piezol activation) undergo pro-T cell differentiation similar to bone marrow (BM)-HSCs. Further, FIG. 7A and FIG. 7B show that iPSC-derived HSCs generated with EHT of CD34+ cells from differentiated iPSCs (in this case involving Piezol activation) undergo T cell differentiation and can be activated with CD3/CD28 beads similar to BM-HSCs. FIG. 8 shows that iPSC-derived HSCs (in this case generated with Piezol activation) can differentiate to functional T cells, as demonstrated by INFγ expression upon stimulation with CD3/CD28 beads. Together, these results demonstrate that iPSC-derived HSCs (i.e., derived with EHT of CD34+ cells from differentiated iPSCs) enhances HSC ability to further differentiate to hematopoietic lineages ex vivo, such as progenitor T cells and functional T cells.

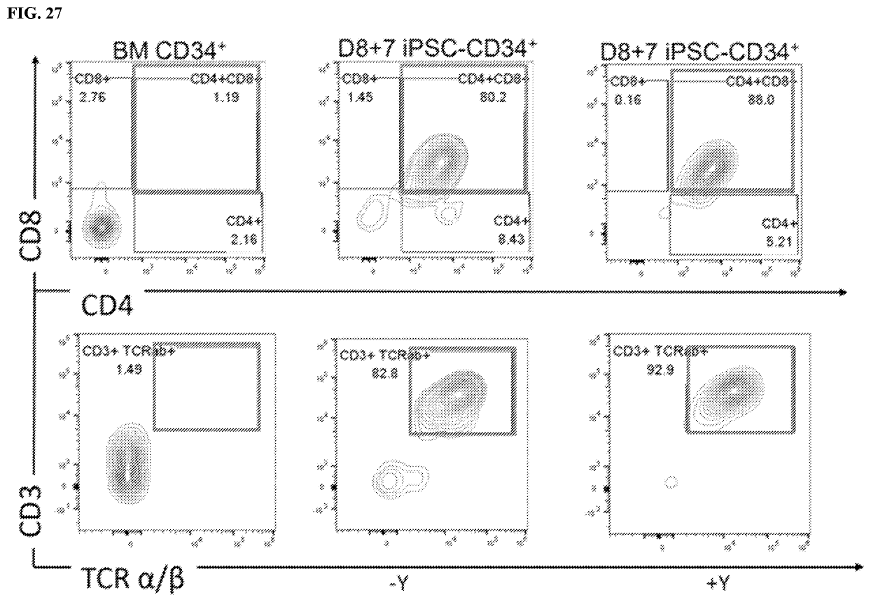

FIG. 27 shows that HSCs generated according to this disclosure (labeled as D8+7 iPSC-CD34+) successfully differentiate into CD4+CD8+ (“double positive”) T cells as well as TCR α/β T cells. The methods of the present disclosure substantially outperform bone marrow CD34+ cells for T cell maturation. FIG. 27 shows results with (“+Y”) and without (“−Y”) Yoda1 during HSC formation.

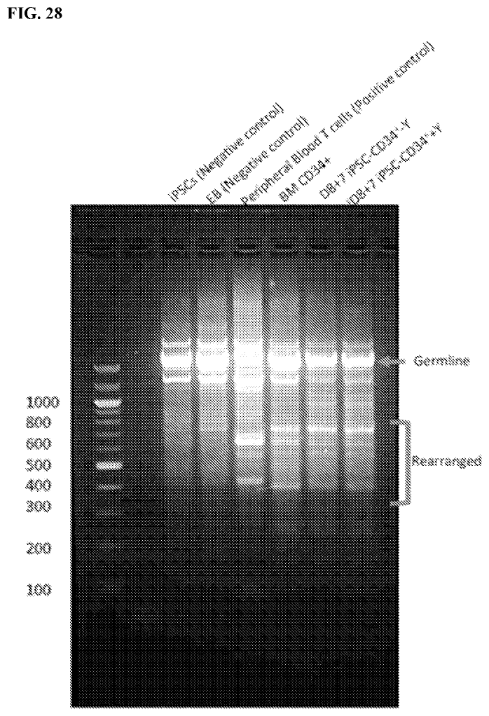

FIG. 28 shows that HSCs generated according to this disclosure (D8+7 iPSC-CD34+ cells (+ or − Yoda1) successfully rearrange TCR, and outperform bone marrow CD34+ cells. Shown are iPSC and EB negative controls, Peripheral Blood T cells as positive control, T cells generated from BM CD34+ cells, and T cells generated according to this instant disclosure with and without Yoda1.

### Example 3: CCR5 Deletion

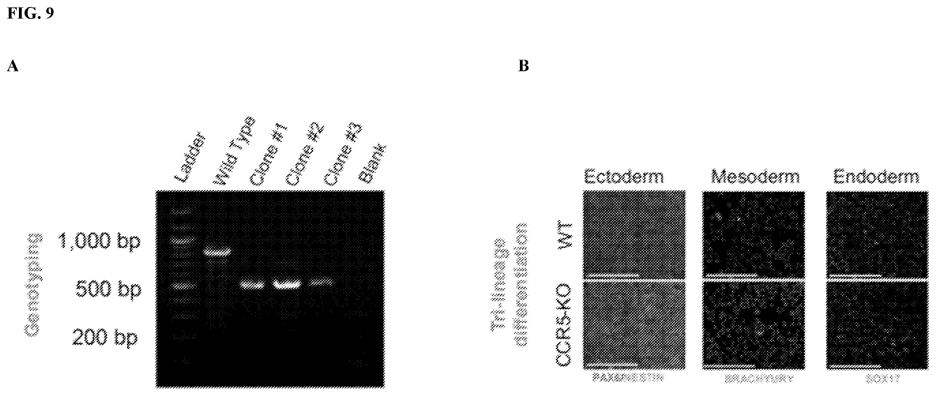

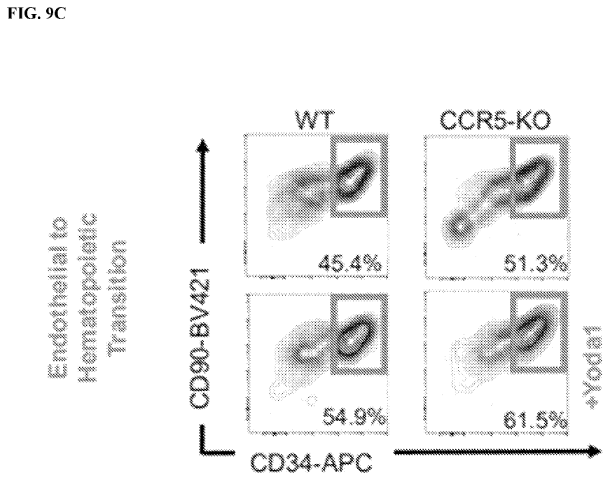

FIG. 9A shows generation of three CCR5-knockout (KO) iPSC clones. As shown in FIG. 9B, the CCR5-KO does not affect the iPSC pluripotency. Further, as shown in FIG. 9C, CCR5-KO does not affect the ability of cells to undergo the endothelial-to hematopoietic transition.

### Example 4: CD33 Deletion

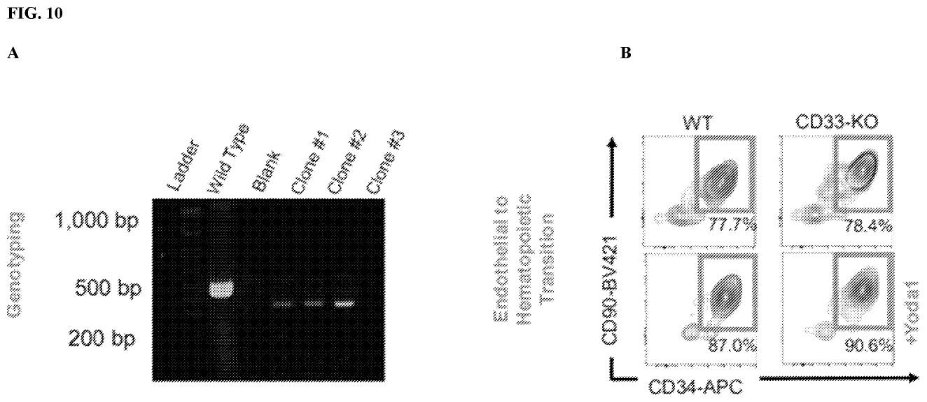

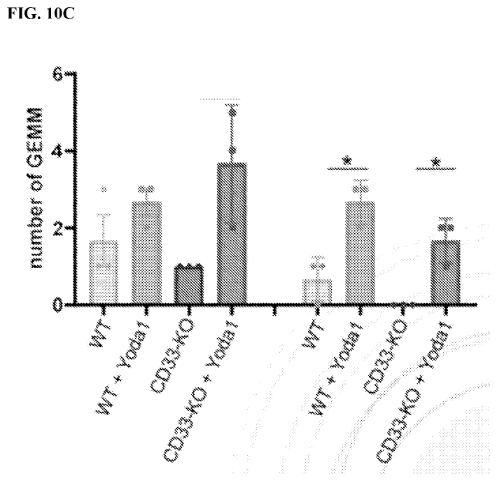

FIG. 10A shows generation of three CD33-KO iPSC clones. As shown in FIG. 10B, CD33-KO does not affect the ability of cells to undergo the endothelial-to hematopoietic transition. Further, CD33-KO does not affect the ability of cells to generate self-renewing HSCs (FIG. 10C).

### Example 5: HLA Editing

According to the disclosure, cells can be HLA-modified by CRISPR-Cas9 using one or more of the following sgRNA comprising one or more spacer sequences shown in the following tables. gRNA were designed using the following parameters: Target sequences are Exon 1 (to stop translation) and Exon 2 (to affect peptide groove) of each variant; PAM motifs with 3′NGG, and target length of 20 nucleotides. Candidate sgRNAs are evaluated for potential off-target editing.
TABLE 2A: HLA-A 01:01 and 02:01
TABLE 2B: HLA-A 02:05 and 03:01
TABLE 2C: HLA-A 11:01 and 23:01
TABLE 2D: HLA-A 25:1 and 26:1
TABLE 2E: HLA-A 29:02 and 30:1
TABLE 2F: HLA-A 30:2 and 31:1
TABLE 2G: HLA-A 33:1
TABLE 3A: HLA-B 07:02 and 08:01
TABLE 3B: HLA-B 13:02 and 14:02
TABLE 3C: HLA-B 15:01 and 18:01
TABLE 3D: HLA-B 35:01 and 38:01
TABLE 3E: HLA-B 40:01 and 44:02
TABLE 3F: HLA-B 44:03 and 50:01
TABLE 3G: HLA-B 52:01 and 57:01
TABLE 4A: HLA-DQB1 02:01 and 03:01
TABLE 4B: HLA-DQB1 03:02 and 03:03
TABLE 4C: HLA-DQB1 05:01 and 05:03
TABLE 4D: HLA-DQB1 06:01 and 06:03
TABLE 4E: HLA-DQB1 06:04
TABLE 5A: HLA-DRB1 01:02, 03:01, 01:01
TABLE 5B: HLA-DRB1 04:01 and 04:02
TABLE 5C: HLA-DRB1 04:04 and 07:01
TABLE 5E: HLA-DRB1 13:02 and 14:01
TABLE 5F: HLA-DRB1 15:01 and 15:02
TABLE 6: HLA-A 01:01, 03:01, 29:02, 33:01
TABLE 7: HLA-A 11:01 and 26:01
TABLE 8: HLA-DQB1 02:01, 06:02, 03:03, and 05:01
TABLE 9: HLA-DQB1 06:01 and 06:03
TABLE 10: HLA-A 24:02
TABLE 11: HLA-B 07:02, 44:03, 57:01, and 08:01
TABLE 12: HLA-B 14:02, 50:01, 37:01, and 52:01
TABLE 13: HLA-B 38:01 and 35:01
TABLE 14: HLA-DRB1 03:01, 15:01, 07:01, and 01:02
TABLE 15: HLA-DRB1 10:01, 15:02, 13:01, and 01:01
TABLE 16: HLA-DPB1 01:01, 02:01, 03:01, and 04:01
TABLE 17: HLA-DPB1 04:02, 11:01, 17:01, and 34:01

TABLE 2A
HLA-A 01:01 and 02:01

| 01:01 | | 02:01 | |
|---|---|---|---|
| Exon1 | Exon2 | Exon1 | Exon2 |
| GAGGGTTCGGGGCGCCA | GAAGAAATACCTCATGGA | GAGGGTTCGGGGCGCCA | GGATGTGAAGAAATACCT |
| TGA (SEQ ID NO: 1) | GT (SEQ ID NO: 2) | TGA (SEQ ID NO: 1) | CA (SEQ ID NO: 3) |
| AGTAGCAGGAGGAGGGT | TGAAGAAATACCTCATGG | AGTAGCAGGACGAGGGT | ATTTCTTCACATCCGTGTC |
| TCG (SEQ ID NO: 4) | AG (SEQ ID NO: 5) | TCG (SEQ ID NO: 6) | C (SEQ ID NO: 7) |
| GAGTAGCAGGAGGAGGG | GGATGTGAAGAAATACCT | GAGTAGCAGGACGAGGG | TTCACATCCGTGTCCCGGC |
| TTC (SEQ ID NO: 8) | CA (SEQ ID NO: 3) | TTC (SEQ ID NO: 9) | C (SEQ ID NO: 10) |
| AGAGTAGCAGGAGGAGG | ATTTCTTCACATCCGTGTC | AGAGTAGCAGGACGAGG | TCCGTGTCCCGGCCCGGC |
| GTT (SEQ ID NO: 11) | C (SEQ ID NO: 7) | GTT (SEQ ID NO: 12) | CG (SEQ ID NO: 13) |
| AACCCTCCTCCTGCTACTC | TTCACATCCGTGTCCCGGC | AACCCTCGTCCTGCTACTC | CCGTGTCCCGGCCCGGCC |
| T (SEQ ID NO: 14) | C (SEQ ID NO: 10) | T (SEQ ID NO: 15) | GC (SEQ ID NO: 16) |
| ACCCTCCTCCTGCTACTCT | TCCGTGTCCCGGCCCGGC | ACCCTCGTCCTGCTACTCT | CCCGCGGCCGGGCCGGG |
| C (SEQ ID NO: 17) | CG (SEQ ID NO: 13) | C (SEQ ID NO: 18) | ACA (SEQ ID NO: 19) |
| CCCTCCTCCTGCTACTCTC | CCGTGTCCCGGCCCGGCC | CCCTCGTCCTGCTACTCTC | CGTGTCCCGGCCCGGCCG |
| G (SEQ ID NO: 20) | GC (SEQ ID NO: 16) | G (SEQ ID NO: 21) | CG (SEQ ID NO: 22) |
| CCCCGAGAGTAGCAGGA | CCCGCGGCCGGGCCGGG | CCCCGAGAGTAGCAGGA | GGGCTCCCCGCGGCCGG |
| GGA (SEQ ID NO: 23) | ACA (SEQ ID NO: 19) | CGA (SEQ ID NO: 24) | GCC (SEQ ID NO: 25) |
| CCTCCTCCTGCTACTCTCG | CGTGTCCCGGCCCGGCCG | CCTCGTCCTGCTACTCTCG | GGGGCTCCCCGCGGCCG |
| G (SEQ ID NO: 26) | CG (SEQ ID NO: 22) | G (SEQ ID NO: 27) | GGC (SEQ ID NO: 28) |
| CCCCCGAGAGTAGCAGGA | GGGCTCCCCGCGGCCGGG | CCCCCGAGAGTAGCAGG | AAGCGGGGCTCCCCGCGG |
| GG (SEQ ID NO: 29) | CC (SEQ ID NO: 25) | ACG (SEQ ID NO: 30) | CC (SEQ ID NO: 31) |
| GGGCCCCCGAGAGTAGC | GGGGCTCCCCGCGGCCGG | CCTGCTACTCTCGGGGGC | GAAGCGGGGCTCCCCGCG |
| AGG (SEQ ID NO: 32) | GC (SEQ ID NO: 28) | TC (SEQ ID NO: 33) | GC (SEQ ID NO: 34) |
| CCTGCTACTCTCGGGGGC | AAGCGGGGCTCCCCGCGG | CCAGAGCCCCCGAGAGTA | CGATGAAGCGGGGCTCCC |
| CC (SEQ ID NO: 35) | CC (SEQ ID NO: 31) | GC (SEQ ID NO: 36) | CG (SEQ ID NO: 37) |
| CCAGGGCCCCCGAGAGTA | GAAGCGGGGCTCCCCGCG | CCAGGGCCCCCGAGAGT | GGAGCCCCGCTTCATCGC |
| GC (SEQ ID NO: 38) | GC (SEQ ID NO: 34) | AGC (SEQ ID NO: 39) | AG (SEQ ID NO: 40) |
| CCCTGGCCCTGACCCAGA | CGATGAAGCGGGGCTCCC | TCTGGCCCTGACCCAGAC | GAGCCCCGCTTCATCGCA |
| CC (SEQ ID NO: 41) | CG (SEQ ID NO: 37) | CT (SEQ ID NO: 42) | GT (SEQ ID NO: 43) |
| CCAGGTCTGGGTCAGGGC | GGAGCCCCGCTTCATCGC | GGCCCTGACCCAGACCTG | TAGCCCACTGCGATGAAG |
| CA (SEQ ID NO: 44) | CG (SEQ ID NO: 45) | GG (SEQ ID NO: 46) | CG (SEQ ID NO: 47) |
| CCTGGCCCTGACCCAGAC | GAGCCCCGCTTCATCGCC | | GTAGCCCACTGCGATGAA |
| CT (SEQ ID NO: 48) | GT (SEQ ID NO: 49) | | GC (SEQ ID NO: 50) |
| CCCAGGTCTGGGTCAGGG | TAGCCCACGGCGATGAAG | | CGTAGCCCACTGCGATGA |
| CC (SEQ ID NO: 51) | CG (SEQ ID NO: 52) | | AG (SEQ ID NO: 53) |
| GGCCCTGACCCAGACCTG | GTAGCCCACGGCGATGAA | | CTTCATCGCAGTGGGCTA |
| GG (SEQ ID NO: 46) | GC (SEQ ID NO: 54) | | CG (SEQ ID NO: 55) |
| | CGTAGCCCACGGCGATGA | | TGGACGACACGCAGTTCG |
| | AG (SEQ ID NO: 56) | | TG (SEQ ID NO: 57) |
| | CTTCATCGCCGTGGGCTA | | ACAGCGACGCCGCGAGCC |
| | CG (SEQ ID NO: 58) | | AG (SEQ ID NO: 59) |
| | CGTGTCGTCCACGTAGCC | | CGACGCCGCGAGCCAGA |
| | CA (SEQ ID NO: 60) | | GGA (SEQ ID NO: 61) |
| | TGGACGACACGCAGTTCG | | CGGCTCCATCCTCTGGCTC |
| | TG (SEQ ID NO: 57) | | G (SEQ ID NO: 62) |
| | CGACGCCGCGAGCCAGAA | | CGAGCCAGAGGATGGAG |
| | GA (SEQ ID NO: 63) | | CCG (SEQ ID NO: 64) |
| | CGGCTCCATCTTCTGGCTC | | GAGCCAGAGGATGGAGC |
| | G (SEQ ID NO: 65) | | CGC (SEQ ID NO: 66) |
| | CGAGCCAGAAGATGGAG | | GCGCCCGCGGCTCCATCC |
| | CCG (SEQ ID NO: 67) | | TC (SEQ ID NO: 68) |
| | GAGCCAGAAGATGGAGC | | GGATGGAGCCGCGGGCG |
| | CGC (SEQ ID NO: 69) | | CCG (SEQ ID NO: 70) |
| | GCGCCCGCGGCTCCATCT | | TGCTCTATCCACGGCGCC |
| | TC (SEQ ID NO: 71) | | CG (SEQ ID NO: 72) |
| | AGATGGAGCCGCGGGCG | | GCGGGCGCCGTGGATAG |
| | CCG (SEQ ID NO: 73) | | AGC (SEQ ID NO: 74) |
| | TGCTCTATCCACGGCGCCC | | GGCGCCGTGGATAGAGC |
| | G (SEQ ID NO: 72) | | AGG (SEQ ID NO: 75) |
| | GCGGGCGCCGTGGATAG | | GCGCCGTGGATAGAGCA |
| | AGC (SEQ ID NO: 74) | | GGA (SEQ ID NO: 76) |
| | GGCGCCGTGGATAGAGCA | | GGACCCTCCTGCTCTATCC |
| | GG (SEQ ID NO: 75) | | A (SEQ ID NO: 77) |
| | GCGCCGTGGATAGAGCAG | | GTGGATAGAGCAGGAGG |
| | GA (SEQ ID NO: 76) | | GTC (SEQ ID NO: 78) |
| | CGCCGTGGATAGAGCAGG | | AGCAGGAGGGTCCGGAG |
| | AG (SEQ ID NO: 79) | | TAT (SEQ ID NO: 80) |
| | GGCCCCTCCTGCTCTATCC | | GCAGGAGGGTCCGGAGT |
| | A (SEQ ID NO: 81) | | ATT (SEQ ID NO: 82) |
| | GTGGATAGAGCAGGAGG | | GAGGGTCCGGAGTATTG |
| | GGC (SEQ ID NO: 83) | | GGA (SEQ ID NO: 84) |
| | AGCAGGAGGGGCCGGAG | | AGGGTCCGGAGTATTGG |
| | TAT (SEQ ID NO: 85) | | GAC (SEQ ID NO: 86) |
| | GCAGGAGGGGCCGGAGT | | GGGTCCGGAGTATTGGG |
| | ATT (SEQ ID NO: 87) | | ACG (SEQ ID NO: 88) |
| | GGGGCCGGAGTATTGGG | | GTCTCCCCGTCCCAATACT |
| | ACC (SEQ ID NO: 89) | | C (SEQ ID NO: 90) |
| | GTCTCCTGGTCCCAATACT | | AGTATTGGGACGGGGAG |
| | C (SEQ ID NO: 91) | | ACA (SEQ ID NO: 92) |
| | AGTATTGGGACCAGGAGA | | CGGGGAGACACGGAAAG |
| | CA (SEQ ID NO: 93) | | TGA (SEQ ID NO: 94) |
| | CCAGGAGACACGGAATAT | | CACTCGGTGAGTCTGTGA |
| | GA (SEQ ID NO: 95) | | GT (SEQ ID NO: 96) |
| | CCTTCATATTCCGTGTCTC | | CCACTCACAGACTCACCG |
| | C (SEQ ID NO: 97) | | AG (SEQ ID NO: 98) |
| | CGCTCGGTCAGTCTGTGA | | CCACTCGGTGAGTCTGTG |
| | GT (SEQ ID NO: 99) | | AG (SEQ ID NO: 100) |
| | TCGCTCGGTCAGTCTGTG | | ACAGACTCACCGAGTGGA |
| | AG (SEQ ID NO: 101) | | CC (SEQ ID NO: 102) |
| | ACAGACTGACCGAGCGAA | | CAGACTCACCGAGTGGAC |
| | CC (SEQ ID NO: 103) | | CT (SEQ ID NO: 104) |
| | CAGACTGACCGAGCGAAC | | AGACTCACCGAGTGGACC |
| | CT (SEQ ID NO: 105) | | TG (SEQ ID NO: 106) |
| | AGACTGACCGAGCGAACC | | GCAGGGTCCCCAGGTCCA |
| | TG (SEQ ID NO: 107) | | CT (SEQ ID NO: 108) |
| | GCAGGGTCCCCAGGTTCG | | GTGGACCTGGGGACCCTG |
| | CT (SEQ ID NO: 109) | | CG (SEQ ID NO: 110) |
| | GCGAACCTGGGGACCCTG | | AGTAGCCGCGCAGGGTCC |
| | CG (SEQ ID NO: 111) | | CC (SEQ ID NO: 112) |
| | AGTAGCCGCGCAGGGTCC | | CTGGTTGTAGTAGCCGCG |
| | CC (SEQ ID NO: 112) | | CA (SEQ ID NO: 113) |
| | CTGGTTGTAGTAGCCGCG | | TCTGGTTGTAGTAGCCGC |
| | CA (SEQ ID NO: 113) | | GC (SEQ ID NO: 114) |
| | TCTGGTTGTAGTAGCCGC | | CGGCTACTACAACCAGAG |
| | GC (SEQ ID NO: 114) | | CG (SEQ ID NO: 115) |
| | CGGCTACTACAACCAGAG | | |
| | CG (SEQ ID NO: 115) | | |

TABLE 2B
HLA-A 02:05 and 03:01

| 02:05 | | 03:01 | |
|---|---|---|---|
| Exon1 | Exon2 | Exon1 | Exon2 |
| GAGGGTTCGGGGCGCCA | GGAGGTGTAGAAATACCT | GAGGGTTCGGGGCGCCA | GAAGAAATACCTCATGGA |
| TGA (SEQ ID NO: 1) | CA (SEQ ID NO: 116) | TGA (SEQ ID NO: 1) | GT (SEQ ID NO: 2) |
| AGTAGCAGGACGAGGGT | ATTTCTACACCTCCGTGTC | AGTAGCAGGAGGAGGGT | TGAAGAAATACCTCATGG |
| TCG (SEQ ID NO: 6) | C (SEQ ID NO: 117) | TCG (SEQ ID NO: 4) | AG (SEQ ID NO: 5) |
| GAGTAGCAGGACGAGGG | TACACCTCCGTGTCCCGGC | GAGTAGCAGGAGGAGGG | GGATGTGAAGAAATACCT |
| TTC (SEQ ID NO: 9) | C (SEQ ID NO: 118) | TTC (SEQ ID NO: 8) | CA (SEQ ID NO: 3) |
| AGAGTAGCAGGACGAGG | GCGGCCGGGCCGGGACA | AGAGTAGCAGGAGGAGG | ATTTCTTCACATCCGTGTC |
| GTT (SEQ ID NO: 12) | CGG (SEQ ID NO: 119) | GTT (SEQ ID NO: 11) | C (SEQ ID NO: 7) |
| AACCCTCGTCCTGCTACTC | TCCGTGTCCCGGCCCGGC | AACCCTCCTCCTGCTACTC | TTCACATCCGTGTCCCGGC |
| T (SEQ ID NO: 15) | CG (SEQ ID NO: 13) | T (SEQ ID NO: 14) | C (SEQ ID NO: 10) |
| ACCCTCGTCCTGCTACTCT | CCGTGTCCCGGCCCGGCC | ACCCTCCTCCTGCTACTCT | TCCGTGTCCCGGCCCGGC |
| C (SEQ ID NO: 18) | GC (SEQ ID NO: 16) | C (SEQ ID NO: 17) | CG (SEQ ID NO: 13) |
| CCCTCGTCCTGCTACTCTC | CCCGCGGCCGGGCCGGG | CCCTCCTCCTGCTACTCTC | CCGTGTCCCGGCCCGGCC |
| G (SEQ ID NO: 21) | ACA (SEQ ID NO: 19) | G (SEQ ID NO: 20) | GC (SEQ ID NO: 16) |
| CCCCGAGAGTAGCAGGA | CGTGTCCCGGCCCGGCCG | CCCCGAGAGTAGCAGGA | CCCGCGGCCGGGCCGGG |
| CGA (SEQ ID NO: 24) | CG (SEQ ID NO: 22) | GGA (SEQ ID NO: 23) | ACA (SEQ ID NO: 19) |
| CCTCGTCCTGCTACTCTCG | GGGCTCCCCGCGGCCGGG | CCTCCTCCTGCTACTCTCG | CGTGTCCCGGCCCGGCCG |
| G (SEQ ID NO: 27) | CC (SEQ ID NO: 25) | G (SEQ ID NO: 26) | CG (SEQ ID NO: 22) |
| CCCCCGAGAGTAGCAGG | GGGGCTCCCCGCGGCCGG | CCCCCGAGAGTAGCAGGA | GGGCTCCCCGCGGCCGGG |
| ACG (SEQ ID NO: 30) | GC (SEQ ID NO: 28) | GG (SEQ ID NO: 29) | CC (SEQ ID NO: 25) |
| CCTGCTACTCTCGGGGGC | AAGCGGGGCTCCCCGCGG | GGGCCCCCGAGAGTAGC | GGGGCTCCCCGCGGCCGG |
| TC (SEQ ID NO: 33) | CC (SEQ ID NO: 31) | AGG (SEQ ID NO: 32) | GC (SEQ ID NO: 28) |
| CCAGAGCCCCCGAGAGTA | GAAGCGGGGCTCCCCGCG | CCTGCTACTCTCGGGGGC | AAGCGGGGCTCCCCGCGG |
| GC (SEQ ID NO: 36) | GC (SEQ ID NO: 34) | CC (SEQ ID NO: 35) | CC (SEQ ID NO: 31) |
| CCAGGGCCCCCGAGAGT | CGATGAAGCGGGGCTCCC | CCAGGGCCCCCGAGAGTA | GAAGCGGGGCTCCCCGCG |
| AGC (SEQ ID NO: 39) | CG (SEQ ID NO: 37) | GC (SEQ ID NO: 38) | GC (SEQ ID NO: 34) |
| TCTGGCCCTGACCCAGAC | GGAGCCCCGCTTCATCGC | CCCTGGCCCTGACCCAGA | CGATGAAGCGGGGCTCCC |
| CT (SEQ ID NO: 42) | AG (SEQ ID NO: 40) | CC (SEQ ID NO: 41) | CG (SEQ ID NO: 37) |
| GGCCCTGACCCAGACCTG | GAGCCCCGCTTCATCGCA | CCAGGTCTGGGTCAGGGC | GGAGCCCCGCTTCATCGC |
| GG (SEQ ID NO: 46) | GT (SEQ ID NO: 43) | CA (SEQ ID NO: 44) | CG (SEQ ID NO: 45) |
| | TAGCCCACTGCGATGAAG | CCTGGCCCTGACCCAGAC | GAGCCCCGCTTCATCGCC |
| | CG (SEQ ID NO: 47) | CT (SEQ ID NO: 48) | GT (SEQ ID NO: 49) |
| | GTAGCCCACTGCGATGAA | CCCAGGTCTGGGTCAGGG | TAGCCCACGGCGATGAAG |
| | GC (SEQ ID NO: 50) | CC (SEQ ID NO: 51) | CG (SEQ ID NO: 52) |
| | CGTAGCCCACTGCGATGA | GGCCCTGACCCAGACCTG | GTAGCCCACGGCGATGAA |
| | AG (SEQ ID NO: 53) | GG (SEQ ID NO: 46) | GC (SEQ ID NO: 54) |
| | CTTCATCGCAGTGGGCTA | | CGTAGCCCACGGCGATGA |
| | CG (SEQ ID NO: 55) | | AG (SEQ ID NO: 56) |
| | TGGACGACACGCAGTTCG | | CTTCATCGCCGTGGGCTA |
| | TG (SEQ ID NO: 57) | | CG (SEQ ID NO: 58) |
| | TCGACAGCGACGCCGCGA | | CGTGTCGTCCACGTAGCC |
| | GC (SEQ ID NO: 120) | | CA (SEQ ID NO: 60) |
| | ACAGCGACGCCGCGAGCC | | TGGACGACACGCAGTTCG |
| | GG (SEQ ID NO: 121) | | TG (SEQ ID NO: 57) |
| | CGACGCCGCGAGCCGGA | | ACAGCGACGCCGCGAGCC |
| | GGA (SEQ ID NO: 122) | | AG (SEQ ID NO: 59) |
| | CGGCTCCATCCTCCGGCTC | | CGACGCCGCGAGCCAGAG |
| | G (SEQ ID NO: 123) | | GA (SEQ ID NO: 61) |
| | CGAGCCGGAGGATGGAG | | CGGCTCCATCCTCTGGCTC |
| | CCG (SEQ ID NO: 124) | | G (SEQ ID NO: 62) |
| | GAGCCGGAGGATGGAGC | | CGAGCCAGAGGATGGAG |
| | CGC (SEQ ID NO: 125) | | CCG (SEQ ID NO: 64) |
| | GCGCCCGCGGCTCCATCC | | GAGCCAGAGGATGGAGC |
| | TC (SEQ ID NO: 68) | | CGC (SEQ ID NO: 66) |
| | GGATGGAGCCGCGGGCG | | GCGCCCGCGGCTCCATCC |
| | CCG (SEQ ID NO: 70) | | TC (SEQ ID NO: 68) |
| | TGCTCTATCCACGGCGCCC | | GGATGGAGCCGCGGGCG |
| | G (SEQ ID NO: 72) | | CCG (SEQ ID NO: 70) |
| | GCGGGCGCCGTGGATAG | | TGCTCTATCCACGGCGCCC |
| | AGC (SEQ ID NO: 74) | | G (SEQ ID NO: 72) |
| | GGCGCCGTGGATAGAGC | | GCGGGCGCCGTGGATAG |
| | AGG (SEQ ID NO: 75) | | AGC (SEQ ID NO: 74) |
| | GCGCCGTGGATAGAGCA | | GGCGCCGTGGATAGAGCA |
| | GGA (SEQ ID NO: 76) | | GG (SEQ ID NO: 75) |
| | GGACCCTCCTGCTCTATCC | | GCGCCGTGGATAGAGCAG |
| | A (SEQ ID NO: 77) | | GA (SEQ ID NO: 76) |
| | GTGGATAGAGCAGGAGG | | CGCCGTGGATAGAGCAGG |
| | GTC (SEQ ID NO: 78) | | AG (SEQ ID NO: 79) |
| | AGCAGGAGGGTCCGGAG | | GGCCCCTCCTGCTCTATCC |
| | TAT (SEQ ID NO: 80) | | A (SEQ ID NO: 81) |
| | GCAGGAGGGTCCGGAGT | | GTGGATAGAGCAGGAGG |
| | ATT (SEQ ID NO: 82) | | GGC (SEQ ID NO: 83) |
| | GAGGGTCCGGAGTATTGG | | AGCAGGAGGGGCCGGAG |
| | GA (SEQ ID NO: 84) | | TAT (SEQ ID NO: 85) |
| | AGGGTCCGGAGTATTGGG | | GCAGGAGGGGCCGGAGT |
| | AC (SEQ ID NO: 86) | | ATT (SEQ ID NO: 87) |
| | GGGTCCGGAGTATTGGGA | | GGGGCCGGAGTATTGGG |
| | CG (SEQ ID NO: 88) | | ACC (SEQ ID NO: 89) |
| | GTCTCCCCGTCCCAATACT | | GTCTCCTGGTCCCAATACT |
| | C (SEQ ID NO: 90) | | C (SEQ ID NO: 91) |
| | AGTATTGGGACGGGGAG | | AGTATTGGGACCAGGAGA |
| | ACA (SEQ ID NO: 92) | | CA (SEQ ID NO: 93) |
| | CGGGGAGACACGGAAAG | | CCAGGAGACACGGAATGT |
| | TGA (SEQ ID NO: 94) | | GA (SEQ ID NO: 126) |
| | CACTCGGTGAGTCTGTGA | | CCTTCACATTCCGTGTCTC |
| | GT (SEQ ID NO: 96) | | C (SEQ ID NO: 127) |
| | CCACTCACAGACTCACCG | | CACTCGGTCAGTCTGTGA |
| | AG (SEQ ID NO: 98) | | CT (SEQ ID NO: 128) |
| | CCACTCGGTGAGTCTGTG | | CCAGTCACAGACTGACCG |
| | AG (SEQ ID NO: 100) | | AG (SEQ ID NO: 129) |
| | ACAGACTCACCGAGTGGA | | CCACTCGGTCAGTCTGTG |
| | CC (SEQ ID NO: 102) | | AC (SEQ ID NO: 130) |
| | CAGACTCACCGAGTGGAC | | ACAGACTGACCGAGTGGA |
| | CT (SEQ ID NO: 104) | | CC (SEQ ID NO: 131) |
| | AGACTCACCGAGTGGACC | | CAGACTGACCGAGTGGAC |
| | TG (SEQ ID NO: 106) | | CT (SEQ ID NO: 132) |
| | GCAGGGTCCCCAGGTCCA | | AGACTGACCGAGTGGACC |
| | CT (SEQ ID NO: 108) | | TG (SEQ ID NO: 133) |
| | GTGGACCTGGGGACCCTG | | GCAGGGTCCCCAGGTCCA |
| | CG (SEQ ID NO: 110) | | CT (SEQ ID NO: 108) |
| | AGTAGCCGCGCAGGGTCC | | GTGGACCTGGGGACCCTG |
| | CC (SEQ ID NO: 112) | | CG (SEQ ID NO: 110) |
| | CTGGTTGTAGTAGCCGCG | | AGTAGCCGCGCAGGGTCC |
| | CA (SEQ ID NO: 113) | | CC (SEQ ID NO: 112) |
| | TCTGGTTGTAGTAGCCGC | | CTGGTTGTAGTAGCCGCG |
| | GC (SEQ ID NO: 114) | | CA (SEQ ID NO: 113) |
| | CGGCTACTACAACCAGAG | | TCTGGTTGTAGTAGCCGC |
| | CG (SEQ ID NO: 115) | | GC (SEQ ID NO: 114) |
| | | | CGGCTACTACAACCAGAG |
| | | | CG (SEQ ID NO: 115) |

TABLE 2C
HLA-A 11:01 and 23:01

| 11:01 | | 23:01 | |
|---|---|---|---|
| Exon1 | Exon2 | Exon1 | Exon2 |
| GAGGGTTCGGGGCGCCA | GTAGAAATACCTCATGGA | GAGGGTTCGGGGCGCCA | GGAGAAATACCTCATGGA |
| TGA (SEQ ID NO: 1) | GT (SEQ ID NO: 134) | TGA (SEQ ID NO: 1) | GT (SEQ ID NO: 135) |
| AGTAGCAGGAGGAGGGT | TGTAGAAATACCTCATGG | AGTAGCAGGACGAGGGT | TGGAGAAATACCTCATGG |
| TCG (SEQ ID NO: 4) | AG (SEQ ID NO: 136) | TCG (SEQ ID NO: 6) | AG (SEQ ID NO: 137) |
| GAGTAGCAGGAGGAGGG | GGAGGTGTAGAAATACCT | GAGTAGCAGGACGAGGG | GGATGTGGAGAAATACCT |
| TTC (SEQ ID NO: 8) | CA (SEQ ID NO: 116) | TTC (SEQ ID NO: 9) | CA (SEQ ID NO: 138) |
| AGAGTAGCAGGAGGAGG | ATTTCTACACCTCCGTGTC | AGAGTAGCAGGACGAGG | ATTTCTCCACATCCGTGTC |
| GTT (SEQ ID NO: 11) | C (SEQ ID NO: 117) | GTT (SEQ ID NO: 12) | C (SEQ ID NO: 139) |
| AACCCTCCTCCTGCTACTC | TACACCTCCGTGTCCCGGC | AACCCTCGTCCTGCTACTC | TCCACATCCGTGTCCCGGC |
| T (SEQ ID NO: 14) | C (SEQ ID NO: 118) | T (SEQ ID NO: 15) | C (SEQ ID NO: 140) |
| ACCCTCCTCCTGCTACTCT | GCGGCCGGGCCGGGACA | ACCCTCGTCCTGCTACTCT | GCCGGGCCGGGACACGG |
| C (SEQ ID NO: 17) | CGG (SEQ ID NO: 119) | C (SEQ ID NO: 18) | ATG (SEQ ID NO: 141) |
| CCCTCCTCCTGCTACTCTC | TCCGTGTCCCGGCCCGGC | CCCTCGTCCTGCTACTCTC | TCCGTGTCCCGGCCCGGC |
| G (SEQ ID NO: 20) | CG (SEQ ID NO: 13) | G (SEQ ID NO: 21) | CG (SEQ ID NO: 13) |
| CCCCGAGAGTAGCAGGA | CCGTGTCCCGGCCCGGCC | CCCCGAGAGTAGCAGGA | CCGTGTCCCGGCCCGGCC |
| GGA (SEQ ID NO: 23) | GC (SEQ ID NO: 16) | CGA (SEQ ID NO: 24) | GC (SEQ ID NO: 16) |
| CCTCCTCCTGCTACTCTCG | CCCGCGGCCGGGCCGGG | CCTCGTCCTGCTACTCTCG | CCCGCGGCCGGGCCGGG |
| G (SEQ ID NO: 26) | ACA (SEQ ID NO: 19) | G (SEQ ID NO: 27) | ACA (SEQ ID NO: 19) |
| CCCCCGAGAGTAGCAGGA | CGTGTCCCGGCCCGGCCG | CCCCCGAGAGTAGCAGG | CGTGTCCCGGCCCGGCCG |
| GG (SEQ ID NO: 29) | CG (SEQ ID NO: 22) | ACG (SEQ ID NO: 30) | CG (SEQ ID NO: 22) |
| GGGCCCCCGAGAGTAGC | GGGCTCCCCGCGGCCGGG | CCTGCTACTCTCGGGGGC | GGGCTCCCCGCGGCCGGG |
| AGG (SEQ ID NO: 32) | CC (SEQ ID NO: 25) | CC (SEQ ID NO: 35) | CC (SEQ ID NO: 25) |
| CCTGCTACTCTCGGGGGC | GGGGCTCCCCGCGGCCGG | CCAGGGCCCCCGAGAGT | GGGGCTCCCCGCGGCCGG |
| CC (SEQ ID NO: 35) | GC (SEQ ID NO: 28) | AGC (SEQ ID NO: 38) | GC (SEQ ID NO: 28) |
| CCAGGGCCCCCGAGAGTA | AAGCGGGGCTCCCCGCGG | CCCTGGCCCTGACCCAGA | AAGCGGGGCTCCCCGCGG |
| GC (SEQ ID NO: 38) | CC (SEQ ID NO: 31) | CC (SEQ ID NO: 41) | CC (SEQ ID NO: 31) |
| CCCTGGCCCTGACCCAGA | GAAGCGGGGCTCCCCGCG | CCAGGTCTGGGTCAGGG | GAAGCGGGGCTCCCCGCG |
| CC (SEQ ID NO: 41) | GC (SEQ ID NO: 34) | CCA (SEQ ID NO: 44) | GC (SEQ ID NO: 34) |
| CCAGGTCTGGGTCAGGGC | CGATGAAGCGGGGCTCCC | CCTGGCCCTGACCCAGAC | CGATGAAGCGGGGCTCCC |
| CA (SEQ ID NO: 44) | CG (SEQ ID NO: 37) | CT (SEQ ID NO: 48) | CG (SEQ ID NO: 37) |
| CCTGGCCCTGACCCAGAC | GGAGCCCCGCTTCATCGC | CCCAGGTCTGGGTCAGG | GGAGCCCCGCTTCATCGC |
| CT (SEQ ID NO: 48) | CG (SEQ ID NO: 45) | GCC (SEQ ID NO: 51) | CG (SEQ ID NO: 45) |
| CCCAGGTCTGGGTCAGGG | GAGCCCCGCTTCATCGCC | | GAGCCCCGCTTCATCGCC |
| CC (SEQ ID NO: 51) | GT (SEQ ID NO: 49) | | GT (SEQ ID NO: 49) |
| GGCCCTGACCCAGACCTG | TAGCCCACGGCGATGAAG | | TAGCCCACGGCGATGAAG |
| GG (SEQ ID NO: 46) | CG (SEQ ID NO: 52) | | CG (SEQ ID NO: 52) |
| | GTAGCCCACGGCGATGAA | | GTAGCCCACGGCGATGAA |
| | GC (SEQ ID NO: 54) | | GC (SEQ ID NO: 54) |
| | CGTAGCCCACGGCGATGA | | CGTAGCCCACGGCGATGA |
| | AG (SEQ ID NO: 56) | | AG (SEQ ID NO: 56) |
| | CTTCATCGCCGTGGGCTA | | CTTCATCGCCGTGGGCTA |
| | CG (SEQ ID NO: 58) | | CG (SEQ ID NO: 58) |
| | CGTGTCGTCCACGTAGCC | | CGTGTCGTCCACGTAGCC |
| | CA (SEQ ID NO: 60) | | CA (SEQ ID NO: 60) |
| | TGGACGACACGCAGTTCG | | TGGACGACACGCAGTTCG |
| | TG (SEQ ID NO: 57) | | TG (SEQ ID NO: 57) |
| | ACAGCGACGCCGCGAGCC | | ACAGCGACGCCGCGAGCC |
| | AG (SEQ ID NO: 59) | | AG (SEQ ID NO: 59) |
| | CGACGCCGCGAGCCAGAG | | CGACGCCGCGAGCCAGAG |
| | GA (SEQ ID NO: 61) | | GA (SEQ ID NO: 61) |
| | CGGCTCCATCCTCTGGCTC | | CGGCTCCATCCTCTGGCTC |
| | G (SEQ ID NO: 62) | | G (SEQ ID NO: 62) |
| | CGAGCCAGAGGATGGAG | | CGAGCCAGAGGATGGAG |
| | CCG (SEQ ID NO: 64) | | CCG (SEQ ID NO: 64) |
| | GAGCCAGAGGATGGAGC | | GAGCCAGAGGATGGAGC |
| | CGC (SEQ ID NO: 66) | | CGC (SEQ ID NO: 66) |
| | GCGCCCGCGGCTCCATCC | | GCGCCCGCGGCTCCATCC |
| | TC (SEQ ID NO: 68) | | TC (SEQ ID NO: 68) |
| | GGATGGAGCCGCGGGCG | | GGATGGAGCCGCGGGCG |
| | CCG (SEQ ID NO: 70) | | CCG (SEQ ID NO: 70) |
| | TGCTCTATCCACGGCGCCC | | TGCTCTATCCACGGCGCCC |
| | G (SEQ ID NO: 72) | | G (SEQ ID NO: 72) |
| | GCGGGCGCCGTGGATAG | | GCGGGCGCCGTGGATAG |
| | AGC (SEQ ID NO: 74) | | AGC (SEQ ID NO: 74) |
| | GGCGCCGTGGATAGAGC | | GGCGCCGTGGATAGAGC |
| | AGG (SEQ ID NO: 75) | | AGG (SEQ ID NO: 75) |
| | GCGCCGTGGATAGAGCA | | GCGCCGTGGATAGAGCA |
| | GGA (SEQ ID NO: 76) | | GGA (SEQ ID NO: 76) |
| | CGCCGTGGATAGAGCAG | | CGCCGTGGATAGAGCAG |
| | GAG (SEQ ID NO: 79) | | GAG (SEQ ID NO: 79) |
| | GGCCCCTCCTGCTCTATCC | | GGCCCCTCCTGCTCTATCC |
| | A (SEQ ID NO: 81) | | A (SEQ ID NO: 81) |
| | GTGGATAGAGCAGGAGG | | GTGGATAGAGCAGGAGG |
| | GGC (SEQ ID NO: 83) | | GGC (SEQ ID NO: 83) |
| | AGCAGGAGGGGCCGGAG | | AGCAGGAGGGGCCGGAG |
| | TAT (SEQ ID NO: 85) | | TAT (SEQ ID NO: 85) |
| | GCAGGAGGGGCCGGAGT | | GCAGGAGGGGCCGGAGT |
| | ATT (SEQ ID NO: 87) | | ATT (SEQ ID NO: 87) |
| | GGGGCCGGAGTATTGGG | | GGGGCCGGAGTATTGGG |
| | ACC (SEQ ID NO: 89) | | ACG (SEQ ID NO: 142) |
| | GTCTCCTGGTCCCAATACT | | GTCTCCTCGTCCCAATACT |
| | C (SEQ ID NO: 91) | | C (SEQ ID NO: 143) |
| | AGTATTGGGACCAGGAGA | | GAGTATTGGGACGAGGA |
| | CA (SEQ ID NO: 93) | | GAC (SEQ ID NO: 144) |
| | CCAGGAGACACGGAATGT | | AGTATTGGGACGAGGAG |
| | GA (SEQ ID NO: 126) | | ACA (SEQ ID NO: 145) |
| | CCTTCACATTCCGTGTCTC | | CGAGGAGACAGGGAAAG |
| | C (SEQ ID NO: 127) | | TGA (SEQ ID NO: 146) |
| | CACTCGGTCAGTCTGTGA | | CTCTCGGTCAGTCTGTGA |
| | CT (SEQ ID NO: 128) | | GT (SEQ ID NO: 147) |
| | CCAGTCACAGACTGACCG | | TCTCTCGGTCAGTCTGTGA |
| | AG (SEQ ID NO: 129) | | G (SEQ ID NO: 148) |
| | CCACTCGGTCAGTCTGTG | | AGACTGACCGAGAGAACC |
| | AC (SEQ ID NO: 130) | | TG (SEQ ID NO: 149) |
| | ACAGACTGACCGAGTGGA | | GCGCGATCCGCAGGTTCT |
| | CC (SEQ ID NO: 131) | | CT (SEQ ID NO: 150) |
| | CAGACTGACCGAGTGGAC | | AGTAGCGGAGCGCGATCC |
| | CT (SEQ ID NO: 132) | | GC (SEQ ID NO: 151) |
| | AGACTGACCGAGTGGACC | | CCGCTACTACAACCAGAG |
| | TG (SEQ ID NO: 133) | | CG (SEQ ID NO: 152) |
| | GCAGGGTCCCCAGGTCCA | | CCTCGCTCTGGTTGTAGTA |
| | CT (SEQ ID NO: 108) | | G (SEQ ID NO: 153) |
| | GTGGACCTGGGGACCCTG | | |
| | CG (SEQ ID NO: 110) | | |
| | AGTAGCCGCGCAGGGTCC | | |
| | CC (SEQ ID NO: 112) | | |
| | CTGGTTGTAGTAGCCGCG | | |
| | CA (SEQ ID NO: 113) | | |
| | TCTGGTTGTAGTAGCCGC | | |
| | GC (SEQ ID NO: 114) | | |
| | CGGCTACTACAACCAGAG | | |
| | CG (SEQ ID NO: 115) | | |

TABLE 2D
HLA-A 25:1 and 26:1

| 25:1 | | 26:1 | |
|---|---|---|---|
| Exon1 | Exon2 | Exon1 | Exon2 |
| GAGGGTTCGGGGCGCCA | GTAGAAATACCTCATGGA | GAGGGTTCGGGGCGCCA | GTAGAAATACCTCATGGA |
| TGA (SEQ ID NO: 1) | GT (SEQ ID NO: 134) | TGA (SEQ ID NO: 1) | GT (SEQ ID NO: 134) |
| AGTAGCAGGACGAGGGT | TGTAGAAATACCTCATGG | AGTAGCAGGACGAGGGT | TGTAGAAATACCTCATGG |
| TCG (SEQ ID NO: 6) | AG (SEQ ID NO: 136) | TCG (SEQ ID NO: 6) | AG (SEQ ID NO: 136) |
| GAGTAGCAGGACGAGGG | GGAGGTGTAGAAATACCT | GAGTAGCAGGACGAGGG | GGAGGTGTAGAAATACCT |
| TTC (SEQ ID NO: 9) | CA (SEQ ID NO: 116) | TTC (SEQ ID NO: 9) | CA (SEQ ID NO: 116) |
| AGAGTAGCAGGACGAGG | ATTTCTACACCTCCGTGTC | AGAGTAGCAGGACGAGG | ATTTCTACACCTCCGTGTC |
| GTT (SEQ ID NO: 12) | C (SEQ ID NO: 117) | GTT (SEQ ID NO: 12) | C (SEQ ID NO: 117) |
| AACCCTCGTCCTGCTACTC | TACACCTCCGTGTCCCGGC | AACCCTCGTCCTGCTACTC | TACACCTCCGTGTCCCGGC |
| T (SEQ ID NO: 15) | C (SEQ ID NO: 118) | T (SEQ ID NO: 15) | C (SEQ ID NO: 118) |
| ACCCTCGTCCTGCTACTCT | GCGGCCGGGCCGGGACA | ACCCTCGTCCTGCTACTCT | GCGGCCGGGCCGGGACA |
| C (SEQ ID NO: 18) | CGG (SEQ ID NO: 119) | C (SEQ ID NO: 18) | CGG (SEQ ID NO: 119) |
| CCCTCGTCCTGCTACTCTC | TCCGTGTCCCGGCCCGGC | CCCTCGTCCTGCTACTCTC | TCCGTGTCCCGGCCCGGC |
| G (SEQ ID NO: 21) | CG (SEQ ID NO: 13) | G (SEQ ID NO: 21) | CG (SEQ ID NO: 13) |
| CCCCGAGAGTAGCAGGA | CCGTGTCCCGGCCCGGCC | CCCCGAGAGTAGCAGGA | CCGTGTCCCGGCCCGGCC |
| CGA (SEQ ID NO: 24) | GC (SEQ ID NO: 16) | CGA (SEQ ID NO: 24) | GC (SEQ ID NO: 16) |
| CCTCGTCCTGCTACTCTCG | CCCGCGGCCGGGCCGGG | CCTCGTCCTGCTACTCTCG | CCCGCGGCCGGGCCGGG |
| G (SEQ ID NO: 27) | ACA (SEQ ID NO: 19) | G (SEQ ID NO: 27) | ACA (SEQ ID NO: 19) |
| CCCCCGAGAGTAGCAGG | CGTGTCCCGGCCCGGCCG | CCCCCGAGAGTAGCAGG | CGTGTCCCGGCCCGGCCG |
| ACG (SEQ ID NO: 30) | CG (SEQ ID NO: 22) | ACG (SEQ ID NO: 30) | CG (SEQ ID NO: 22) |
| CCTGCTACTCTCGGGGGC | GGGCTCCCCGCGGCCGGG | CCTGCTACTCTCGGGGGC | GGGCTCCCCGCGGCCGGG |
| CC (SEQ ID NO: 35) | CC (SEQ ID NO: 25) | CC (SEQ ID NO: 35) | CC (SEQ ID NO: 25) |
| CCAGGGCCCCCGAGAGT | GGGGCTCCCCGCGGCCGG | CCAGGGCCCCCGAGAGT | GGGGCTCCCCGCGGCCGG |
| AGC | GC (SEQ ID NO: 28) | AGC (SEQ ID NO: 38) | GC (SEQ ID NO: 28) |
| CCCTGGCCCTGACCCAGA | AAGCGGGGCTCCCCGCGG | CCCTGGCCCTGACCCAGA | AAGCGGGGCTCCCCGCGG |
| CC (SEQ ID NO: 41) | CC (SEQ ID NO: 31) | CC (SEQ ID NO: 41) | CC (SEQ ID NO: 31) |
| CCAGGTCTGGGTCAGGG | GAAGCGGGGCTCCCCGCG | CCAGGTCTGGGTCAGGG | GAAGCGGGGCTCCCCGCG |
| CCA (SEQ ID NO: 44) | GC (SEQ ID NO: 34) | CCA (SEQ ID NO: 44) | GC (SEQ ID NO: 34) |
| CCTGGCCCTGACCCAGAC | CGATGAAGCGGGGCTCCC | CCTGGCCCTGACCCAGAC | CGATGAAGCGGGGCTCCC |
| CT (SEQ ID NO: 48) | CG (SEQ ID NO: 37) | CT (SEQ ID NO: 48) | CG (SEQ ID NO: 37) |
| CCCAGGTCTGGGTCAGG | GGAGCCCCGCTTCATCGC | CCCAGGTCTGGGTCAGG | GGAGCCCCGCTTCATCGC |
| GCC (SEQ ID NO: 51) | CG (SEQ ID NO: 45) | GCC (SEQ ID NO: 51) | CG (SEQ ID NO: 45) |
| GGCCCTGACCCAGACCTG | GAGCCCCGCTTCATCGCC | GGCCCTGACCCAGACCTG | GAGCCCCGCTTCATCGCC |
| GG (SEQ ID NO: 46) | GT (SEQ ID NO: 49) | GG (SEQ ID NO: 46) | GT (SEQ ID NO: 49) |
| | TAGCCCACGGCGATGAAG | | TAGCCCACGGCGATGAAG |
| | CG (SEQ ID NO: 52) | | CG (SEQ ID NO: 52) |
| | GTAGCCCACGGCGATGAA | | GTAGCCCACGGCGATGAA |
| | GC (SEQ ID NO: 54) | | GC (SEQ ID NO: 54) |
| | CGTAGCCCACGGCGATGA | | CGTAGCCCACGGCGATGA |
| | AG (SEQ ID NO: 56) | | AG (SEQ ID NO: 56) |
| | CTTCATCGCCGTGGGCTAC | | CTTCATCGCCGTGGGCTAC |
| | G (SEQ ID NO: 58) | | G (SEQ ID NO: 58) |
| | CGTGTCGTCCACGTAGCC | | CGTGTCGTCCACGTAGCC |
| | CA (SEQ ID NO: 60) | | CA (SEQ ID NO: 60) |
| | TGGACGACACGCAGTTCG | | TGGACGACACGCAGTTCG |
| | TG (SEQ ID NO: 57) | | TG (SEQ ID NO: 57) |
| | ACAGCGACGCCGCGAGCC | | ACAGCGACGCCGCGAGCC |
| | AG (SEQ ID NO: 59) | | AG (SEQ ID NO: 59) |
| | CGACGCCGCGAGCCAGAG | | CGACGCCGCGAGCCAGAG |
| | GA (SEQ ID NO: 61) | | GA (SEQ ID NO: 61) |
| | CGGCTCCATCCTCTGGCTC | | CGGCTCCATCCTCTGGCTC |
| | G (SEQ ID NO: 62) | | G (SEQ ID NO: 62) |
| | CGAGCCAGAGGATGGAG | | CGAGCCAGAGGATGGAG |
| | CCG (SEQ ID NO: 64) | | CCG (SEQ ID NO: 64) |
| | GAGCCAGAGGATGGAGC | | GAGCCAGAGGATGGAGC |
| | CGC (SEQ ID NO: 66) | | CGC (SEQ ID NO: 66) |
| | GCGCCCGCGGCTCCATCC | | GCGCCCGCGGCTCCATCC |
| | TC (SEQ ID NO: 68) | | TC (SEQ ID NO: 68) |
| | GGATGGAGCCGCGGGCG | | GGATGGAGCCGCGGGCG |
| | CCG (SEQ ID NO: 70) | | CCG (SEQ ID NO: 70) |
| | TGCTCTATCCACGGCGCCC | | TGCTCTATCCACGGCGCCC |
| | G (SEQ ID NO: 72) | | G (SEQ ID NO: 72) |
| | GCGGGCGCCGTGGATAG | | GCGGGCGCCGTGGATAG |
| | AGC (SEQ ID NO: 74) | | AGC (SEQ ID NO: 74) |
| | GGCGCCGTGGATAGAGCA | | GGCGCCGTGGATAGAGCA |
| | GG (SEQ ID NO: 75) | | GG (SEQ ID NO: 75) |
| | GCGCCGTGGATAGAGCAG | | GCGCCGTGGATAGAGCAG |
| | GA (SEQ ID NO: 76) | | GA (SEQ ID NO: 76) |
| | CGCCGTGGATAGAGCAGG | | CGCCGTGGATAGAGCAGG |
| | AG (SEQ ID NO: 79) | | AG (SEQ ID NO: 79) |
| | GGCCCCTCCTGCTCTATCC | | GGCCCCTCCTGCTCTATCC |
| | A (SEQ ID NO: 81) | | A (SEQ ID NO: 81) |
| | GTGGATAGAGCAGGAGG | | GTGGATAGAGCAGGAGG |
| | GGC (SEQ ID NO: 83) | | GGC (SEQ ID NO: 83) |
| | AGCAGGAGGGGCCGGAG | | AGCAGGAGGGGCCGGAG |
| | TAT (SEQ ID NO: 85) | | TAT (SEQ ID NO: 85) |
| | GCAGGAGGGGCCGGAGT | | GCAGGAGGGGCCGGAGT |
| | ATT (SEQ ID NO: 87) | | ATT (SEQ ID NO: 87) |
| | AGGGGCCGGAGTATTGG | | AGGGGCCGGAGTATTGG |
| | GAC (SEQ ID NO: 154) | | GAC (SEQ ID NO: 154) |
| | GTGTTCCGGTCCCAATACT | | GTGTTCCGGTCCCAATACT |
| | C (SEQ ID NO: 155) | | C (SEQ ID NO: 155) |
| | AGTATTGGGACCGGAACA | | AGTATTGGGACCGGAACA |
| | CA (SEQ ID NO: 156) | | CA (SEQ ID NO: 156) |
| | CCGGAACACACGGAATGT | | CCGGAACACACGGAATGT |
| | GA (SEQ ID NO: 157) | | GA (SEQ ID NO: 157) |
| | CCTTCACATTCCGTGTGTT | | CCTTCACATTCCGTGTGTT |
| | C (SEQ ID NO: 158) | | C (SEQ ID NO: 158) |
| | CTCTCGGTCAGTCTGTGA | | CGCTCGGTCAGTCTGTGA |
| | GT (SEQ ID NO: 147) | | GT (SEQ ID NO: 99) |
| | TCTCTCGGTCAGTCTGTGA | | TCGCTCGGTCAGTCTGTG |
| | G (SEQ ID NO: 148) | | AG (SEQ ID NO: 101) |
| | AGACTGACCGAGAGAGCC | | ACAGACTGACCGAGCGAA |
| | TG (SEQ ID NO: 159) | | CC (SEQ ID NO: 103) |
| | GCGCGATCCGCAGGCTCT | | CAGACTGACCGAGCGAAC |
| | CT (SEQ ID NO: 160) | | CT (SEQ ID NO: 105) |
| | AGTAGCGGAGCGCGATCC | | AGACTGACCGAGCGAACC |
| | GC (SEQ ID NO: 151) | | TG (SEQ ID NO: 107) |
| | CCGCTACTACAACCAGAG | | GCAGGGTCCCCAGGTTCG |
| | CG (SEQ ID NO: 152) | | CT (SEQ ID NO: 109) |
| | CCTCGCTCTGGTTGTAGTA | | GCGAACCTGGGGACCCTG |
| | G (SEQ ID NO: 153) | | CG (SEQ ID NO: 111) |
| | | | AGTAGCCGCGCAGGGTCC |
| | | | CC (SEQ ID NO: 112) |
| | | | CTGGTTGTAGTAGCCGCG |
| | | | CA (SEQ ID NO: 113) |
| | | | TCTGGTTGTAGTAGCCGC |
| | | | GC (SEQ ID NO: 114) |
| | | | CGGCTACTACAACCAGAG |
| | | | CG (SEQ ID NO: 115) |

TABLE 2E
HLA-A 29:02 and 30:1

| 29:02 | | 30:1 | |
|---|---|---|---|
| Exon1 | Exon2 | Exon1 | Exon2 |
| GAGGGTTCGGGGCGCCA | GGTGAAATACCTCATGGA | GAGGGTTCGGGGCGCCA | GGAGAAATACCTCATGGA |
| TGA (SEQ ID NO: 1) | GT (SEQ ID NO: 161) | TGA (SEQ ID NO: 1) | GT (SEQ ID NO: 135) |
| AGTAGCAGGAGGAGGGT | TGGTGAAATACCTCATGG | AGTAGCAGGAGGAGGGT | TGGAGAAATACCTCATGG |
| TCG (SEQ ID NO: 4) | AG (SEQ ID NO: 162) | TCG (SEQ ID NO: 4) | AG (SEQ ID NO: 137) |
| GAGTAGCAGGAGGAGGG | GGATGTGGTGAAATACCT | GAGTAGCAGGAGGAGGG | GGATGTGGAGAAATACCT |
| TTC (SEQ ID NO: 8) | CA (SEQ ID NO: 163) | TTC (SEQ ID NO: 8) | CA (SEQ ID NO: 138) |
| AGAGTAGCAGGAGGAGG | ATTTCACCACATCCGTGTC | AGAGTAGCAGGAGGAGG | ATTTCTCCACATCCGTGTC |
| GTT (SEQ ID NO: 11) | C (SEQ ID NO: 164) | GTT (SEQ ID NO: 11) | C (SEQ ID NO: 139) |
| AACCCTCCTCCTGCTACTC | ACCACATCCGTGTCCCGG | AACCCTCCTCCTGCTACTC | TCCACATCCGTGTCCCGG |
| T (SEQ ID NO: 14) | CC (SEQ ID NO: 165) | T (SEQ ID NO: 14) | CC (SEQ ID NO: 140) |
| ACCCTCCTCCTGCTACTCT | GCCGGGCCGGGACACGG | ACCCTCCTCCTGCTACTCT | GCCGGGCCGGGACACGG |
| T (SEQ ID NO: 166) | ATG (SEQ ID NO: 141) | C (SEQ ID NO: 17) | ATG (SEQ ID NO: 141) |
| CCCTCCTCCTGCTACTCTT | TCCGTGTCCCGGCCCGGC | CCCTCCTCCTGCTACTCTC | TCCGTGTCCCGGCCCGGC |
| G (SEQ ID NO: 167) | CG (SEQ ID NO: 13) | G (SEQ ID NO: 20) | AG (SEQ ID NO: 168) |
| CCCCAAGAGTAGCAGGA | CCGTGTCCCGGCCCGGCC | CCCCGAGAGTAGCAGGA | TCCACTGCCGGGCCGGGA |
| GGA (SEQ ID NO: 169) | GC (SEQ ID NO: 16) | GGA (SEQ ID NO: 23) | CA (SEQ ID NO: 170) |
| CCTCCTCCTGCTACTCTTG | CCCGCGGCCGGGCCGGG | CCTCCTCCTGCTACTCTCG | GGGCTCTCCACTGCCGGG |
| G (SEQ ID NO: 171) | ACA (SEQ ID NO: 19) | G (SEQ ID NO: 26) | CC (SEQ ID NO: 172) |
| CCCCCAAGAGTAGCAGGA | CGTGTCCCGGCCCGGCCG | CCCCCGAGAGTAGCAGGA | GGGGCTCTCCACTGCCGG |
| GG (SEQ ID NO: 173) | CG (SEQ ID NO: 22) | GG (SEQ ID NO: 29) | GC (SEQ ID NO: 174) |
| GGGCCCCCAAGAGTAGCA | GGGCTCCCCGCGGCCGGG | GGGCCCCCGAGAGTAGC | AAGCGGGGCTCTCCACTG |
| GG (SEQ ID NO: 175) | CC (SEQ ID NO: 25) | AGG (SEQ ID NO: 32) | CC (SEQ ID NO: 176) |
| CCTGCTACTCTTGGGGGC | GGGGCTCCCCGCGGCCGG | CCTGCTACTCTCGGGGGC | GAAGCGGGGCTCTCCACT |
| CC (SEQ ID NO: 177) | GC (SEQ ID NO: 28) | CC (SEQ ID NO: 35) | GC (SEQ ID NO: 178) |
| CCAGGGCCCCCAAGAGTA | AAGCGGGGCTCCCCGCGG | CCAGGGCCCCCGAGAGTA | AGAGCCCCGCTTCATCGC |
| GC (SEQ ID NO: 179) | CC (SEQ ID NO: 31) | GC (SEQ ID NO: 38) | AG (SEQ ID NO: 180) |
| CCCTGGCCCTGACCCAGA | GAAGCGGGGCTCCCCGCG | CCCTGGCCCTGACCCAGA | GAGCCCCGCTTCATCGCA |
| CC (SEQ ID NO: 41) | GC (SEQ ID NO: 34) | CC (SEQ ID NO: 41) | GT (SEQ ID NO: 43) |
| CCAGGTCTGGGTCAGGGC | CGATGAAGCGGGGCTCCC | CCAGGTCTGGGTCAGGGC | TAGCCCACTGCGATGAAG |
| CA (SEQ ID NO: 44) | CG (SEQ ID NO: 37) | CA (SEQ ID NO: 44) | CG (SEQ ID NO: 47) |
| CCTGGCCCTGACCCAGAC | GGAGCCCCGCTTCATCGC | CCTGGCCCTGACCCAGAC | GTAGCCCACTGCGATGAA |
| CT (SEQ ID NO: 48) | CG (SEQ ID NO: 45) | CT (SEQ ID NO: 48) | GC (SEQ ID NO: 50) |
| CCCAGGTCTGGGTCAGGG | GAGCCCCGCTTCATCGCC | CCCAGGTCTGGGTCAGGG | CGTAGCCCACTGCGATGA |
| CC (SEQ ID NO: 51) | GT (SEQ ID NO: 49) | CC (SEQ ID NO: 51) | AG (SEQ ID NO: 53) |
| GGCCCTGACCCAGACCTG | TAGCCCACGGCGATGAAG | GGCCCTGACCCAGACCTG | CTTCATCGCAGTGGGCTA |
| GG (SEQ ID NO: 46) | CG (SEQ ID NO: 52) | GG (SEQ ID NO: 46) | CG (SEQ ID NO: 55) |
| | GTAGCCCACGGCGATGAA | | TGGACGACACGCAGTTCG |
| | GC (SEQ ID NO: 54) | | TG (SEQ ID NO: 57) |
| | CGTAGCCCACGGCGATGA | | ACAGCGACGCCGCGAGCC |
| | AG (SEQ ID NO: 56) | | AG (SEQ ID NO: 59) |
| | CTTCATCGCCGTGGGCTA | | CGACGCCGCGAGCCAGA |
| | CG (SEQ ID NO: 58) | | GGA (SEQ ID NO: 61) |
| | CGTGTCGTCCACGTAGCC | | CGGCTCCATCCTCTGGCTC |
| | CA (SEQ ID NO: 60) | | G (SEQ ID NO: 62) |
| | TGGACGACACGCAGTTCG | | CGAGCCAGAGGATGGAG |
| | TG (SEQ ID NO: 57) | | CCG (SEQ ID NO: 64) |
| | ACAGCGACGCCGCGAGCC | | GAGCCAGAGGATGGAGC |
| | AG (SEQ ID NO: 59) | | CGC (SEQ ID NO: 66) |
| | CGACGCCGCGAGCCAGAG | | GCGCCCGCGGCTCCATCC |
| | GA (SEQ ID NO: 61) | | TC (SEQ ID NO: 68) |
| | CGGCTCCATCCTCTGGCTC | | GGATGGAGCCGCGGGCG |
| | G (SEQ ID NO: 62) | | CCG (SEQ ID NO: 70) |
| | CGAGCCAGAGGATGGAG | | TGCTCTATCCACGGCGCC |
| | CCG (SEQ ID NO: 64) | | CG (SEQ ID NO: 72) |
| | GAGCCAGAGGATGGAGC | | GCGGGCGCCGTGGATAG |
| | CGC (SEQ ID NO: 66) | | AGC (SEQ ID NO: 74) |
| | GTGCCCGCGGCTCCATCC | | CGCCGTGGATAGAGCAG |
| | TC (SEQ ID NO: 181) | | GAG (SEQ ID NO: 79) |
| | GGATGGAGCCGCGGGCA | | GGCCTCTCCTGCTCTATCC |
| | CCG (SEQ ID NO: 182) | | A (SEQ ID NO: 183) |
| | TGCTCTATCCACGGTGCCC | | AGCAGGAGAGGCCTGAG |
| | G (SEQ ID NO: 184) | | TAT (SEQ ID NO: 185) |
| | GCGGGCACCGTGGATAG | | GCAGGAGAGGCCTGAGT |
| | AGC (SEQ ID NO: 186) | | ATT (SEQ ID NO: 187) |
| | GGCACCGTGGATAGAGCA | | GAGGCCTGAGTATTGGGA |
| | GG (SEQ ID NO: 188) | | CC (SEQ ID NO: 189) |
| | GCACCGTGGATAGAGCAG | | GTCTCCTGGTCCCAATACT |
| | GA (SEQ ID NO: 190) | | C (SEQ ID NO: 91) |
| | CACCGTGGATAGAGCAGG | | AGTATTGGGACCAGGAG |
| | AG (SEQ ID NO: 191) | | ACA (SEQ ID NO: 93) |
| | GGCCCCTCCTGCTCTATCC | | CCAGGAGACACGGAATGT |
| | A (SEQ ID NO: 81) | | GA (SEQ ID NO: 126) |
| | GTGGATAGAGCAGGAGG | | CCTTCACATTCCGTGTCTC |
| | GGC (SEQ ID NO: 83) | | C (SEQ ID NO: 127) |
| | AGCAGGAGGGGCCGGAG | | CACTCGGTCAGTCTGTGA |
| | TAT (SEQ ID NO: 85) | | CT (SEQ ID NO: 128) |
| | GCAGGAGGGGCCGGAGT | | CCAGTCACAGACTGACCG |
| | ATT (SEQ ID NO: 87) | | AG (SEQ ID NO: 129) |
| | GTCTGCAGGTCCCAATAC | | CCACTCGGTCAGTCTGTG |
| | TC (SEQ ID NO: 192) | | AC (SEQ ID NO: 130) |
| | AGTATTGGGACCTGCAGA | | ACAGACTGACCGAGTGGA |
| | CA (SEQ ID NO: 193) | | CC (SEQ ID NO: 131) |
| | CCTGCAGACACGGAATGT | | CAGACTGACCGAGTGGAC |
| | GA (SEQ ID NO: 194) | | CT (SEQ ID NO: 132) |
| | CCTTCACATTCCGTGTCTG | | AGACTGACCGAGTGGACC |
| | C (SEQ ID NO: 195) | | TG (SEQ ID NO: 133) |
| | CGCTCGGTCAGTCTGTGA | | GCAGGGTCCCCAGGTCCA |
| | CT (SEQ ID NO: 196) | | CT (SEQ ID NO: 108) |
| | TCGCTCGGTCAGTCTGTG | | GTGGACCTGGGGACCCTG |
| | AC (SEQ ID NO: 197) | | CG (SEQ ID NO: 110) |
| | ACAGACTGACCGAGCGAA | | AGTAGCCGCGCAGGGTCC |
| | CC (SEQ ID NO: 103) | | CC (SEQ ID NO: 112) |
| | CAGACTGACCGAGCGAAC | | CTGGTTGTAGTAGCCGCG |
| | CT (SEQ ID NO: 105) | | CA (SEQ ID NO: 113) |
| | AGACTGACCGAGCGAACC | | TCTGGTTGTAGTAGCCGC |
| | TG (SEQ ID NO: 107) | | GC (SEQ ID NO: 114) |
| | GCAGGGTCCCCAGGTTCG | | CGGCTACTACAACCAGAG |
| | CT (SEQ ID NO: 109) | | CG (SEQ ID NO: 115) |
| | GCGAACCTGGGGACCCTG | | |
| | CG (SEQ ID NO: 111) | | |
| | AGTAGCCGCGCAGGGTCC | | |
| | CC (SEQ ID NO: 112) | | |
| | CTGGTTGTAGTAGCCGCG | | |
| | CA (SEQ ID NO: 113) | | |
| | TCTGGTTGTAGTAGCCGC | | |
| | GC (SEQ ID NO: 114) | | |
| | CGGCTACTACAACCAGAG | | |
| | CG (SEQ ID NO: 115) | | |

TABLE 2F
HLA-A 30:2 and 31:1

| 30:2 | | 31:1 | |
|---|---|---|---|
| Exon1 | Exon2 | Exon1 | Exon2 |
| GAGGGTTCGGGGCGCCA | GGAGAAATACCTCATGGA | GAGGGTTCGGGGCGCCA | GGTGAAATACCTCATGGA |
| TGA (SEQ ID NO: 1) | GT (SEQ ID NO: 135) | TGA (SEQ ID NO: 1) | GT (SEQ ID NO: 161) |
| AGTAGCAGGAGGAGGGT | TGGAGAAATACCTCATGG | AGTAGCAGGAGGAGGGT | TGGTGAAATACCTCATGG |
| TCG (SEQ ID NO: 4) | AG (SEQ ID NO: 137) | TCG (SEQ ID NO: 4) | AG (SEQ ID NO: 162) |
| GAGTAGCAGGAGGAGGG | GGATGTGGAGAAATACCT | GAGTAGCAGGAGGAGGG | GGATGTGGTGAAATACCT |
| TTC (SEQ ID NO: 8) | CA (SEQ ID NO: 138) | TTC (SEQ ID NO: 8) | CA (SEQ ID NO: 163) |
| AGAGTAGCAGGAGGAGG | ATTTCTCCACATCCGTGTC | AGAGTAGCAGGAGGAGG | ATTTCACCACATCCGTGTC |
| GTT (SEQ ID NO: 11) | C (SEQ ID NO: 139) | GTT (SEQ ID NO: 11) | C (SEQ ID NO: 164) |
| AACCCTCCTCCTGCTACTC | TCCACATCCGTGTCCCGG | AACCCTCCTCCTGCTACTC | ACCACATCCGTGTCCCGG |
| T (SEQ ID NO: 14) | CC (SEQ ID NO: 140) | T (SEQ ID NO: 14) | CC (SEQ ID NO: 165) |
| ACCCTCCTCCTGCTACTCT | GCCGGGCCGGGACACGG | ACCCTCCTCCTGCTACTCT | GCCGGGCCGGGACACGG |
| C (SEQ ID NO: 17) | ATG (SEQ ID NO: 141) | T (SEQ ID NO: 166) | ATG (SEQ ID NO: 141) |
| CCCTCCTCCTGCTACTCTC | TCCGTGTCCCGGCCCGGC | CCCTCCTCCTGCTACTCTT | TCCGTGTCCCGGCCCGGC |
| G (SEQ ID NO: 20) | AG (SEQ ID NO: 168) | G (SEQ ID NO: 167) | CG (SEQ ID NO: 13) |
| CCCCGAGAGTAGCAGGA | TCCACTGCCGGGCCGGGA | CCCCAAGAGTAGCAGGA | CCGTGTCCCGGCCCGGCC |
| GGA (SEQ ID NO: 23) | CA (SEQ ID NO: 170) | GGA (SEQ ID NO: 169) | GC (SEQ ID NO: 16) |
| CCTCCTCCTGCTACTCTCG | GGGCTCTCCACTGCCGGG | CCTCCTCCTGCTACTCTTG | CCCGCGGCCGGGCCGGG |
| G (SEQ ID NO: 26) | CC (SEQ ID NO: 172) | G (SEQ ID NO: 171) | ACA (SEQ ID NO: 19) |
| CCCCCGAGAGTAGCAGGA | GGGGCTCTCCACTGCCGG | CCCCCAAGAGTAGCAGGA | CGTGTCCCGGCCCGGCCG |
| GG (SEQ ID NO: 29) | GC (SEQ ID NO: 174) | GG (SEQ ID NO: 173) | CG (SEQ ID NO: 22) |
| GGGCCCCCGAGAGTAGC | AAGCGGGGCTCTCCACTG | GGGCCCCCAAGAGTAGCA | GGGCTCCCCGCGGCCGG |
| AGG (SEQ ID NO: 32) | CC (SEQ ID NO: 176) | GG (SEQ ID NO: 175) | GCC (SEQ ID NO: 25) |
| CCTGCTACTCTCGGGGGC | GAAGCGGGGCTCTCCACT | CCTGCTACTCTTGGGGGC | GGGGCTCCCCGCGGCCG |
| CC (SEQ ID NO: 35) | GC (SEQ ID NO: 178) | CC (SEQ ID NO: 177) | GGC (SEQ ID NO: 28) |
| CCAGGGCCCCCGAGAGTA | AGAGCCCCGCTTCATCGC | CCAGGGCCCCCAAGAGTA | AAGCGGGGCTCCCCGCG |
| GC (SEQ ID NO: 38) | AG (SEQ ID NO: 180) | GC (SEQ ID NO: 179) | GCC (SEQ ID NO: 31) |
| CCCTGGCCCTGACCCAGA | GAGCCCCGCTTCATCGCA | CCCTGGCCCTGACCCAGA | GAAGCGGGGCTCCCCGC |
| CC (SEQ ID NO: 41) | GT (SEQ ID NO: 43) | CC (SEQ ID NO: 41) | GGC (SEQ ID NO: 34) |
| CCAGGTCTGGGTCAGGGC | TAGCCCACTGCGATGAAG | CCAGGTCTGGGTCAGGGC | CGATGAAGCGGGGCTCCC |
| CA (SEQ ID NO: 44) | CG (SEQ ID NO: 47) | CA (SEQ ID NO: 44) | CG (SEQ ID NO: 37) |
| CCTGGCCCTGACCCAGAC | GTAGCCCACTGCGATGAA | CCTGGCCCTGACCCAGAC | GGAGCCCCGCTTCATCGC |
| CT (SEQ ID NO: 48) | GC (SEQ ID NO: 50) | CT (SEQ ID NO: 48) | CG (SEQ ID NO: 45) |
| CCCAGGTCTGGGTCAGGG | CGTAGCCCACTGCGATGA | CCCAGGTCTGGGTCAGGG | GAGCCCCGCTTCATCGCC |
| CC (SEQ ID NO: 51) | AG (SEQ ID NO: 53) | CC (SEQ ID NO: 51) | GT (SEQ ID NO: 49) |
| GGCCCTGACCCAGACCTG | CTTCATCGCAGTGGGCTA | GGCCCTGACCCAGACCTG | TAGCCCACGGCGATGAAG |
| GG (SEQ ID NO: 46) | CG (SEQ ID NO: 55) | GG (SEQ ID NO: 46) | CG (SEQ ID NO: 52) |
| | TGGACGACACGCAGTTCG | | GTAGCCCACGGCGATGAA |
| | TG (SEQ ID NO: 57) | | GC (SEQ ID NO: 54) |
| | ACAGCGACGCCGCGAGCC | | CGTAGCCCACGGCGATGA |
| | AG (SEQ ID NO: 59) | | AG (SEQ ID NO: 56) |
| | CGACGCCGCGAGCCAGA | | CTTCATCGCCGTGGGCTA |
| | GGA (SEQ ID NO: 61) | | CG (SEQ ID NO: 58) |
| | CGGCTCCATCCTCTGGCTC | | CGTGTCGTCCACGTAGCC |
| | G (SEQ ID NO: 62) | | CA (SEQ ID NO: 60) |
| | CGAGCCAGAGGATGGAG | | TGGACGACACGCAGTTCG |
| | CCG (SEQ ID NO: 64) | | TG (SEQ ID NO: 57) |
| | GAGCCAGAGGATGGAGC | | ACAGCGACGCCGCGAGCC |
| | CGC (SEQ ID NO: 66) | | AG (SEQ ID NO: 59) |
| | GCGCCCGCGGCTCCATCC | | CGACGCCGCGAGCCAGA |
| | TC (SEQ ID NO: 68) | | GGA (SEQ ID NO: 61) |
| | GGATGGAGCCGCGGGCG | | CGGCTCCATCCTCTGGCTC |
| | CCG (SEQ ID NO: 70) | | G (SEQ ID NO: 62) |
| | TGCTCTATCCACGGCGCC | | CGAGCCAGAGGATGGAG |
| | CG (SEQ ID NO: 72) | | CCG (SEQ ID NO: 64) |
| | GCGGGCGCCGTGGATAG | | GAGCCAGAGGATGGAGC |
| | AGC (SEQ ID NO: 74) | | CGC (SEQ ID NO: 66) |
| | CGCCGTGGATAGAGCAG | | GCGCCCGCGGCTCCATCC |
| | GAG (SEQ ID NO: 79) | | TC (SEQ ID NO: 68) |
| | GGCCTCTCCTGCTCTATCC | | GGATGGAGCCGCGGGCG |
| | A (SEQ ID NO: 183) | | CCG (SEQ ID NO: 70) |
| | AGCAGGAGAGGCCTGAG | | TGCTCTATCCACGGCGCC |
| | TAT (SEQ ID NO: 185) | | CG (SEQ ID NO: 72) |
| | GCAGGAGAGGCCTGAGT | | GCGGGCGCCGTGGATAG |
| | ATT (SEQ ID NO: 187) | | AGC (SEQ ID NO: 74) |
| | GAGGCCTGAGTATTGGGA | | CGCCGTGGATAGAGCAG |
| | CC (SEQ ID NO: 189) | | GAG (SEQ ID NO: 79) |
| | GTCTCCTGGTCCCAATACT | | GGCCTCTCCTGCTCTATCC |
| | C (SEQ ID NO: 91) | | A (SEQ ID NO: 183) |
| | AGTATTGGGACCAGGAG | | AGCAGGAGAGGCCTGAG |
| | ACA (SEQ ID NO: 93) | | TAT (SEQ ID NO: 185) |
| | CCAGGAGACACGGAATGT | | GCAGGAGAGGCCTGAGT |
| | GA (SEQ ID NO: 126) | | ATT (SEQ ID NO: 187) |
| | CCTTCACATTCCGTGTCTC | | GAGGCCTGAGTATTGGGA |
| | C (SEQ ID NO: 127) | | CC (SEQ ID NO: 189) |
| | CTCTCGGTCAGTCTGTGA | | GTCTCCTGGTCCCAATACT |
| | GT (SEQ ID NO: 147) | | C (SEQ ID NO: 91) |
| | TCTCTCGGTCAGTCTGTGA | | JAGTATTGGGACCAGGAG |
| | G (SEQ ID NO: 148) | | ACA (SEQ ID NO: 93) |
| | ACAGACTGACCGAGAGAA | | CCAGGAGACACGGAATGT |
| | CC (SEQ ID NO: 198) | | GA (SEQ ID NO: 126) |
| | CAGACTGACCGAGAGAAC | | CCTTCACATTCCGTGTCTC |
| | CT (SEQ ID NO: 199) | | C (SEQ ID NO: 127) |
| | AGACTGACCGAGAGAACC | | CACTCGGTCAATCTGTGA |
| | TG (SEQ ID NO: 149) | | GT (SEQ ID NO: 200) |
| | GCAGGGTCCCCAGGTTCT | | CCACTCACAGATTGACCG |
| | CT (SEQ ID NO: 201) | | AG (SEQ ID NO: 202) |
| | GAGAACCTGGGGACCCTG | | CCACTCGGTCAATCTGTG |
| | CG (SEQ ID NO: 203) | | AG (SEQ ID NO: 204) |
| | AGTAGCCGCGCAGGGTCC | | ACAGATTGACCGAGTGGA |
| | CC (SEQ ID NO: 112) | | CC (SEQ ID NO: 205) |
| | CTGGTTGTAGTAGCCGCG | | CAGATTGACCGAGTGGAC |
| | CA (SEQ ID NO: 113) | | CT (SEQ ID NO: 206) |
| | TCTGGTTGTAGTAGCCGC | | AGATTGACCGAGTGGACC |
| | GC (SEQ ID NO: 114) | | TG (SEQ ID NO: 207) |
| | CGGCTACTACAACCAGAG | | GCAGGGTCCCCAGGTCCA |
| | CG (SEQ ID NO: 115) | | CT (SEQ ID NO: 108) |
| | | | GTGGACCTGGGGACCCTG |
| | | | CG (SEQ ID NO: 110) |
| | | | AGTAGCCGCGCAGGGTCC |
| | | | CC (SEQ ID NO: 112) |
| | | | CTGGTTGTAGTAGCCGCG |
| | | | CA (SEQ ID NO: 113) |
| | | | TCTGGTTGTAGTAGCCGC |
| | | | GC (SEQ ID NO: 114) |
| | | | CGGCTACTACAACCAGAG |
| | | | CG (SEQ ID NO: 115) |

TABLE 2G
HLA-A 33:1
33:1

| Exon1 | Exon2 |
|---|---|
| GAGGGTTCGGGGCGCCATGA (SEQ ID NO: 1) | GGTGAAATACCTCATGGAGT (SEQ ID NO: 161) |
| AGTAGCAGGAGGAGGGTTCG (SEQ ID NO: 4) | TGGTGAAATACCTCATGGAG (SEQ ID NO: 162) |
| GAGTAGCAGGAGGAGGGTTC (SEQ ID NO: 8) | GGATGTGGTGAAATACCTCA (SEQ ID NO: 163) |
| AGAGTAGCAGGAGGAGGGTT (SEQ ID NO: 11) | ATTTCACCACATCCGTGTCC (SEQ ID NO: 164) |
| AACCCTCCTCCTGCTACTCT (SEQ ID NO: 14) | ACCACATCCGTGTCCCGGCC (SEQ ID NO: 165) |
| ACCCTCCTCCTGCTACTCTT (SEQ ID NO: 166) | GCCGGGCCGGGACACGGATG (SEQ ID NO: 141) |
| CCCTCCTCCTGCTACTCTTG (SEQ ID NO: 167) | TCCGTGTCCCGGCCCGGCCG (SEQ ID NO: 13) |
| CCCCAAGAGTAGCAGGAGGA (SEQ ID NO: 169) | CCGTGTCCCGGCCCGGCCGC (SEQ ID NO: 16) |
| CCTCCTCCTGCTACTCTTGG (SEQ ID NO: 171) | CCCGCGGCCGGGCCGGGACA (SEQ ID NO: 19) |
| CCCCCAAGAGTAGCAGGAGG (SEQ ID NO: 173) | CGTGTCCCGGCCCGGCCGCG (SEQ ID NO: 22) |
| GGGCCCCCAAGAGTAGCAGG (SEQ ID NO: 175) | GGGCTCCCCGCGGCCGGGCC (SEQ ID NO: 25) |
| CCTGCTACTCTTGGGGGCCC (SEQ ID NO: 177) | GGGGCTCCCCGCGGCCGGGC (SEQ ID NO: 28) |
| CCAGGGCCCCCAAGAGTAGC (SEQ ID NO: 179) | AAGCGGGGCTCCCCGCGGCC (SEQ ID NO: 31) |
| CCCTGGCCCTGACCCAGACC (SEQ ID NO: 41) | GAAGCGGGGCTCCCCGCGGC (SEQ ID NO: 34) |
| CCAGGTCTGGGTCAGGGCCA (SEQ ID NO: 44) | CGATGAAGCGGGGCTCCCCG (SEQ ID NO: 37) |
| CCTGGCCCTGACCCAGACCT (SEQ ID NO: 48) | GGAGCCCCGCTTCATCGCCG (SEQ ID NO: 45) |
| CCCAGGTCTGGGTCAGGGCC (SEQ ID NO: 51) | GAGCCCCGCTTCATCGCCGT (SEQ ID NO: 49) |
| GGCCCTGACCCAGACCTGGG (SEQ ID NO: 46) | TAGCCCACGGCGATGAAGCG (SEQ ID NO: 52) |
| | GTAGCCCACGGCGATGAAGC (SEQ ID NO: 54) |
| | CGTAGCCCACGGCGATGAAG (SEQ ID NO: 56) |
| | CTTCATCGCCGTGGGCTACG (SEQ ID NO: 58) |
| | CGTGTCGTCCACGTAGCCCA (SEQ ID NO: 60) |
| | TGGACGACACGCAGTTCGTG (SEQ ID NO: 57) |
| | ACAGCGACGCCGCGAGCCAG (SEQ ID NO: 59) |
| | CGACGCCGCGAGCCAGAGGA (SEQ ID NO: 61) |
| | CGGCTCCATCCTCTGGCTCG (SEQ ID NO: 62) |
| | CGAGCCAGAGGATGGAGCCG (SEQ ID NO: 64) |
| | GAGCCAGAGGATGGAGCCGC (SEQ ID NO: 66) |
| | GCGCCCGCGGCTCCATCCTC (SEQ ID NO: 68) |
| | GGATGGAGCCGCGGGCGCCG (SEQ ID NO: 70) |
| | TGCTCTATCCACGGCGCCCG (SEQ ID NO: 72) |
| | GCGGGCGCCGTGGATAGAGC (SEQ ID NO: 74) |
| | GGCGCCGTGGATAGAGCAGG (SEQ ID NO: 75) |
| | GCGCCGTGGATAGAGCAGGA (SEQ ID NO: 76) |
| | CGCCGTGGATAGAGCAGGAG (SEQ ID NO: 79) |
| | GGCCCCTCCTGCTCTATCCA (SEQ ID NO: 81) |
| | GTGGATAGAGCAGGAGGGGC (SEQ ID NO: 83) |
| | AGCAGGAGGGGCCGGAGTAT (SEQ ID NO: 85) |
| | GCAGGAGGGGCCGGAGTATT (SEQ ID NO: 87) |
| | AGGGGCCGGAGTATTGGGAC (SEQ ID NO: 154) |
| | GTGTTCCGGTCCCAATACTC (SEQ ID NO: 155) |
| | AGTATTGGGACCGGAACACA (SEQ ID NO: 156) |
| | CCGGAACACACGGAATGTGA (SEQ ID NO: 157) |
| | CCTTCACATTCCGTGTGTTC (SEQ ID NO: 158) |
| | CACTCGGTCAATCTGTGAGT (SEQ ID NO: 200) |
| | CCACTCACAGATTGACCGAG (SEQ ID NO: 202) |
| | CCACTCGGTCAATCTGTGAG (SEQ ID NO: 204) |
| | ACAGATTGACCGAGTGGACC (SEQ ID NO: 205) |
| | CAGATTGACCGAGTGGACCT (SEQ ID NO: 206) |
| | AGATTGACCGAGTGGACCTG (SEQ ID NO: 207) |
| | GCAGGGTCCCCAGGTCCACT (SEQ ID NO: 108) |
| | GTGGACCTGGGGACCCTGCG (SEQ ID NO: 110) |
| | AGTAGCCGCGCAGGGTCCCC (SEQ ID NO: 112) |
| | CTGGTTGTAGTAGCCGCGCA (SEQ ID NO: 113) |
| | TCTGGTTGTAGTAGCCGCGC (SEQ ID NO: 114) |
| | CGGCTACTACAACCAGAGCG (SEQ ID NO: 115) |
| | TACTACAACCAGAGCGAGGC (SEQ ID NO: 208) |

TABLE 3A
HLA-B 07:02 and 08:01

| 07:02 | | 08:01 | |
|---|---|---|---|
| Exon1 | Exon2 | Exon1 | Exon2 |
| AGCAGCAGGAGGACGGT | GTAGAAATACCTCATGGA | AGCAGCAGGAGGACGGT | GTCGAAATACCTCATGGA |
| TCG (SEQ ID NO: 209) | GT (SEQ ID NO: 134) | TCG (SEQ ID NO: 209) | GT (SEQ ID NO: 220) |
| GAGCAGCAGGAGGACGG | TGTAGAAATACCTCATGG | GAGCAGCAGGAGGACGG | TGTCGAAATACCTCATGG |
| TTC (SEQ ID NO: 210) | AG (SEQ ID NO: 136) | TTC (SEQ ID NO: 210) | AG (SEQ ID NO: 221) |
| AGAGCAGCAGGAGGACG | GGAGGTGTAGAAATACCT | AGAGCAGCAGGAGGACG | GGCGGTGTCGAAATACCT |
| GTT (SEQ ID NO: 211) | CA (SEQ ID NO: 116) | GTT (SEQ ID NO: 211) | CA (SEQ ID NO: 222) |
| AACCGTCCTCCTGCTGCTC | ATTTCTACACCTCCGTGTC | AACCGTCCTCCTGCTGCTC | ATTTCGACACCGCCATGTC |
| T (SEQ ID NO: 212) | C (SEQ ID NO: 117) | T (SEQ ID NO: 212) | C (SEQ ID NO: 223) |
| CGCCGAGAGCAGCAGGA | TACACCTCCGTGTCCCGG | CGCCGAGAGCAGCAGGA | GACACCGCCATGTCCCGG |
| GGA (SEQ ID NO: 213) | CC (SEQ ID NO: 118) | GGA (SEQ ID NO: 213) | CC (SEQ ID NO: 224) |
| CGTCCTCCTGCTGCTCTCG | GCGGCCGGGCCGGGACA | CGTCCTCCTGCTGCTCTCG | GCGGCCGGGCCGGGACA |
| G (SEQ ID NO: 214) | CGG (SEQ ID NO: 119) | G (SEQ ID NO: 214) | TGG (SEQ ID NO: 225) |
| GGGCCGCCGAGAGCAGC | TCCGTGTCCCGGCCCGGC | GGGCCGCCGAGAGCAGC | GCCATGTCCCGGCCCGGC |
| AGG (SEQ ID NO: 215) | CG (SEQ ID NO: 13) | AGG (SEQ ID NO: 215) | CG (SEQ ID NO: 226) |
| CCTGCTGCTCTCGGCGGC | CCGTGTCCCGGCCCGGCC | CCTGCTGCTCTCGGCGGC | CCATGTCCCGGCCCGGCC |
| CC (SEQ ID NO: 216) | GC (SEQ ID NO: 16) | CC (SEQ ID NO: 216) | GC (SEQ ID NO: 227) |
| CCAGGGCCGCCGAGAGC | CCCGCGGCCGGGCCGGG | CCAGGGCCGCCGAGAGC | CCCGCGGCCGGGCCGGG |
| AGC (SEQ ID NO: 217) | ACA (SEQ ID NO: 19) | AGC (SEQ ID NO: 217) | ACA (SEQ ID NO: 19) |
| CCCTGGCCCTGACCGAGA | CGTGTCCCGGCCCGGCCG | CCCTGGCCCTGACCGAGA | CATGTCCCGGCCCGGCCG |
| CC (SEQ ID NO: 218) | CG (SEQ ID NO: 22) | CC (SEQ ID NO: 218) | CG (SEQ ID NO: 228) |
| CCAGGTCTCGGTCAGGGC | GGGCTCCCCGCGGCCGG | CCAGGTCTCGGTCAGGGC | GGGCTCCCCGCGGCCGG |
| CA (SEQ ID NO: 219) | GCC (SEQ ID NO: 25) | CA (SEQ ID NO: 219) | GCC (SEQ ID NO: 25) |
| CCTGGCCCTGACCGAGAC | GGGGCTCCCCGCGGCCG | CCTGGCCCTGACCGAGAC | GGGGCTCCCCGCGGCCG |
| CT (SEQ ID NO: 229) | GGC (SEQ ID NO: 28) | CT (SEQ ID NO: 229) | GGC (SEQ ID NO: 28) |
| CCCAGGTCTCGGTCAGGG | AAGCGGGGCTCCCCGCGG | CCCAGGTCTCGGTCAGGG | AAGCGGGGCTCCCCGCGG |
| CC (SEQ ID NO: 230) | CC (SEQ ID NO: 31) | CC (SEQ ID NO: 230) | CC (SEQ ID NO: 31) |
| | GAAGCGGGGCTCCCCGCG | | GAAGCGGGGCTCCCCGCG |
| | GC (SEQ ID NO: 34) | | GC (SEQ ID NO: 34) |
| | AGATGAAGCGGGGCTCCC | | AGATGAAGCGGGGCTCCC |
| | CG (SEQ ID NO: 231) | | CG (SEQ ID NO: 231) |
| | GGAGCCCCGCTTCATCTC | | GGAGCCCCGCTTCATCTC |
| | AG (SEQ ID NO: 232) | | AG (SEQ ID NO: 232) |
| | GAGCCCCGCTTCATCTCA | | GAGCCCCGCTTCATCTCA |
| | GT (SEQ ID NO: 233) | | GT (SEQ ID NO: 233) |
| | TAGCCCACTGAGATGAAG | | TAGCCCACTGAGATGAAG |
| | CG (SEQ ID NO: 234) | | CG (SEQ ID NO: 234) |
| | GTAGCCCACTGAGATGAA | | GTAGCCCACTGAGATGAA |
| | GC (SEQ ID NO: 235) | | GC (SEQ ID NO: 235) |
| | CGTAGCCCACTGAGATGA | | CGTAGCCCACTGAGATGA |
| | AG (SEQ ID NO: 236) | | AG (SEQ ID NO: 236) |
| | CTTCATCTCAGTGGGCTAC | | CTTCATCTCAGTGGGCTAC |
| | G (SEQ ID NO: 237) | | G (SEQ ID NO: 237) |
| | TGGACGACACCCAGTTCG | | TGGACGACACGCAGTTCG |
| | TG (SEQ ID NO: 238) | | TG (SEQ ID NO: 57) |
| | GCTGTCGAACCTCACGAA | | CGACGCCGCGAGTCCGAG |
| | CT (SEQ ID NO: 239) | | AG (SEQ ID NO: 241) |
| | CGCTGTCGAACCTCACGA | | CGGCTCCTCTCTCGGACTC |
| | AC (SEQ ID NO: 240) | | G (SEQ ID NO: 242) |
| | CGACGCCGCGAGTCCGAG | | CGAGTCCGAGAGAGGAG |
| | AG (SEQ ID NO: 241) | | CCG (SEQ ID NO: 243) |
| | CGGCTCCTCTCTCGGACTC | | GAGTCCGAGAGAGGAGC |
| | G (SEQ ID NO: 242) | | CGC (SEQ ID NO: 244) |
| | CGAGTCCGAGAGAGGAG | | GGCGCCCGCGGCTCCTCT |
| | CCG (SEQ ID NO: 243) | | CT (SEQ ID NO: 245) |
| | GAGTCCGAGAGAGGAGC | | GAGAGGAGCCGCGGGCG |
| | CGC (SEQ ID NO: 244) | | CCG (SEQ ID NO: 246) |
| | GGCGCCCGCGGCTCCTCT | | TGCTCTATCCACGGCGCC |
| | CT (SEQ ID NO: 245) | | CG (SEQ ID NO: 72) |
| | GAGAGGAGCCGCGGGCG | | GCGGGCGCCGTGGATAG |
| | CCG (SEQ ID NO: 246) | | AGC (SEQ ID NO: 74) |
| | TGCTCTATCCACGGCGCC | | GGCGCCGTGGATAGAGC |
| | CG (SEQ ID NO: 72) | | AGG (SEQ ID NO: 75) |
| | GCGGGCGCCGTGGATAG | | GCGCCGTGGATAGAGCA |
| | AGC (SEQ ID NO: 74) | | GGA (SEQ ID NO: 76) |
| | GGCGCCGTGGATAGAGC | | CGCCGTGGATAGAGCAG |
| | AGG (SEQ ID NO: 75) | | GAG (SEQ ID NO: 79) |
| | GCGCCGTGGATAGAGCA | | GGCCCCTCCTGCTCTATCC |
| | GGA (SEQ ID NO: 76) | | A (SEQ ID NO: 81) |
| | CGCCGTGGATAGAGCAG | | GTGGATAGAGCAGGAGG |
| | GAG (SEQ ID NO: 79) | | GGC (SEQ ID NO: 83) |
| | GGCCCCTCCTGCTCTATCC | | AGCAGGAGGGGCCGGAG |
| | A (SEQ ID NO: 81) | | TAT (SEQ ID NO: 85) |
| | GTGGATAGAGCAGGAGG | | GCAGGAGGGGCCGGAGT |
| | GGC (SEQ ID NO: 83) | | ATT (SEQ ID NO: 87) |
| | AGCAGGAGGGGCCGGAG | | AGGGGCCGGAGTATTGG |
| | TAT (SEQ ID NO: 85) | | GAC (SEQ ID NO: 154) |
| | GCAGGAGGGGCCGGAGT | | GTGTTCCGGTCCCAATACT |
| | ATT (SEQ ID NO: 87) | | C (SEQ ID NO: 155) |
| | AGGGGCCGGAGTATTGG | | TCTTGAAGATCTGTGTGTT |
| | GAC (SEQ ID NO: 154) | | C (SEQ ID NO: 255) |
| | GTGTTCCGGTCCCAATACT | | CTCTCGGTCAGTCTGTGT |
| | C (SEQ ID NO: 155) | | GT (SEQ ID NO: 256) |
| | CCGGAACACACAGATCTA | | AGACTGACCGAGAGAGCC |
| | CA (SEQ ID NO: 247) | | TG (SEQ ID NO: 159) |
| | CCTTGTAGATCTGTGTGTT | | GCAGGTTCCGCAGGCTCT |
| | C (SEQ ID NO: 248) | | CT (SEQ ID NO: 252) |
| | CACACAGATCTACAAGGC | | GAGAGCCTGCGGAACCTG |
| | CC (SEQ ID NO: 249) | | CG (SEQ ID NO: 253) |
| | CTCTCGGTCAGTCTGTGCC | | AGTAGCCGCGCAGGTTCC |
| | T (SEQ ID NO: 250) | | GC (SEQ ID NO: 254) |
| | TCTCTCGGTCAGTCTGTGC | | TCTGGTTGTAGTAGCCGC |
| | C (SEQ ID NO: 251) | | GC (SEQ ID NO: 114) |
| | AGACTGACCGAGAGAGCC | | CGGCTACTACAACCAGAG |
| | TG (SEQ ID NO: 159) | | CG (SEQ ID NO: 115) |
| | GCAGGTTCCGCAGGCTCT | | |
| | CT (SEQ ID NO: 252) | | |
| | GAGAGCCTGCGGAACCTG | | |
| | CG (SEQ ID NO: 253) | | |
| | AGTAGCCGCGCAGGTTCC | | |
| | GC (SEQ ID NO: 254) | | |
| | TCTGGTTGTAGTAGCCGC | | |
| | GC (SEQ ID NO: 114) | | |
| | CGGCTACTACAACCAGAG | | |
| | CG (SEQ ID NO: 115) | | |

TABLE 3B
HLA-B 13:02 and 14:02

| 13:02 | | 14:02 | |
|---|---|---|---|
| AGCAGCAGGAGGAGGGT | GTAGAAATACCTCATGGA | AGCAGCAGGAGGACGGT | GTAGAAATACCTCATGGA |
| TCG (SEQ ID NO: 257) | GT (SEQ ID NO: 134) | TCG (SEQ ID NO: 209) | GT (SEQ ID NO: 134) |
| GAGCAGCAGGAGGAGGG | TGTAGAAATACCTCATGG | GAGCAGCAGGAGGACGG | TGTAGAAATACCTCATGG |
| TTC (SEQ ID NO: 258) | AG (SEQ ID NO: 136) | TTC (SEQ ID NO: 210) | AG (SEQ ID NO: 136) |
| AGAGCAGCAGGAGGAGG | GGCGGTGTAGAAATACCT | AGAGCAGCAGGAGGACG | GGCGGTGTAGAAATACCT |
| GTT (SEQ ID NO: 259) | CA (SEQ ID NO: 272) | GTT (SEQ ID NO: 211) | CA (SEQ ID NO: 272) |
| GAACCCTCCTCCTGCTGCT | ATTTCTACACCGCCATGTC | AACCGTCCTCCTGCTGCTC | ATTTCTACACCGCCGTGTC |
| C (SEQ ID NO: 260) | C (SEQ ID NO: 273) | T (SEQ ID NO: 212) | C (SEQ ID NO: 305) |
| AACCCTCCTCCTGCTGCTC | TACACCGCCATGTCCCGG | CGCCGAGAGCAGCAGGA | TACACCGCCGTGTCCCGG |
| T (SEQ ID NO: 261) | CC (SEQ ID NO: 274) | GGA (SEQ ID NO: 213) | CC (SEQ ID NO: 306) |
| ACCCTCCTCCTGCTGCTCT | GCGGCCGGGCCGGGACA | CGTCCTCCTGCTGCTCTCG | GCGGCCGGGCCGGGACA |
| G (SEQ ID NO: 262) | TGG (SEQ ID NO: 225) | G (SEQ ID NO: 214) | CGG (SEQ ID NO: 119) |
| CCCTCCTCCTGCTGCTCTG | GCCATGTCCCGGCCCGGC | GGGCCGCCGAGAGCAGC | GCCGTGTCCCGGCCCGGC |
| G (SEQ ID NO: 263) | CG (SEQ ID NO: 226) | AGG (SEQ ID NO: 215) | CG (SEQ ID NO: 307) |
| CCCCCAGAGCAGCAGGA | CCATGTCCCGGCCCGGCC | CCTGCTGCTCTCGGCGGC | CCGTGTCCCGGCCCGGCC |
| GGA (SEQ ID NO: 264) | GC (SEQ ID NO: 227) | CC (SEQ ID NO: 216) | GC (SEQ ID NO: 16) |
| CCTCCTCCTGCTGCTCTGG | CCCGCGGCCGGGCCGGG | CCAGGGCCGCCGAGAGC | CCCGCGGCCGGGCCGGG |
| G (SEQ ID NO: 265) | ACA (SEQ ID NO: 19) | AGC (SEQ ID NO: 217) | ACA (SEQ ID NO: 19) |
| CCCCCCAGAGCAGCAGGA | CATGTCCCGGCCCGGCCG | CCCTGGCCCTGACCGAGA | CGTGTCCCGGCCCGGCCG |
| GG (SEQ ID NO: 266) | CG (SEQ ID NO: 228) | CC (SEQ ID NO: 218) | CG (SEQ ID NO: 22) |
| CTGCCCCCCAGAGCAGCA | GGGCTCCCCGCGGCCGG | CCAGGTCTCGGTCAGGGC | GGGCTCCCCGCGGCCGGG |
| GG (SEQ ID NO: 267) | GCC (SEQ ID NO: 25) | CA (SEQ ID NO: 219) | CC (SEQ ID NO: 25) |
| CCTGCTGCTCTGGGGGGC | GGGGCTCCCCGCGGCCG | CCTGGCCCTGACCGAGAC | GGGGCTCCCCGCGGCCGG |
| AG (SEQ ID NO: 268) | GGC (SEQ ID NO: 28) | CT (SEQ ID NO: 229) | GC (SEQ ID NO: 28) |
| CCACTGCCCCCCAGAGCA | AAGCGGGGCTCCCCGCG | CCCAGGTCTCGGTCAGGG | AAGCGGGGCTCCCCGCGG |
| GC (SEQ ID NO: 269) | GCC (SEQ ID NO: 31) | CC (SEQ ID NO: 230) | CC (SEQ ID NO: 31) |
| CAGTGGCCCTGACCGAGA | GAAGCGGGGCTCCCCGC | | GAAGCGGGGCTCCCCGCG |
| CC (SEQ ID NO: 270) | GGC (SEQ ID NO: 34) | | GC (SEQ ID NO: 34) |
| AGTGGCCCTGACCGAGAC | TGATGAAGCGGGGCTCCC | | AGATGAAGCGGGGCTCCC |
| CT (SEQ ID NO: 271) | CG (SEQ ID NO: 275) | | CG (SEQ ID NO: 231) |
| | GGAGCCCCGCTTCATCAC | | GGAGCCCCGCTTCATCTC |
| | CG (SEQ ID NO: 276) | | AG (SEQ ID NO: 232) |
| | GAGCCCCGCTTCATCACC | | GAGCCCCGCTTCATCTCA |
| | GT (SEQ ID NO: 277) | | GT (SEQ ID NO: 233) |
| | TAGCCCACGGTGATGAAG | | TAGCCCACTGAGATGAAG |
| | CG (SEQ ID NO: 278) | | CG (SEQ ID NO: 234) |
| | GTAGCCCACGGTGATGAA | | GTAGCCCACTGAGATGAA |
| | GC (SEQ ID NO: 279) | | GC (SEQ ID NO: 235) |
| | CGTAGCCCACGGTGATGA | | CGTAGCCCACTGAGATGA |
| | AG (SEQ ID NO: 280) | | AG (SEQ ID NO: 236) |
| | CTTCATCACCGTGGGCTA | | CTTCATCTCAGTGGGCTAC |
| | CG (SEQ ID NO: 281) | | G (SEQ ID NO: 237) |
| | GGTGTCGTCCACGTAGCC | | TGGACGACACGCAGTTCG |
| | CA (SEQ ID NO: 282) | | TG (SEQ ID NO: 57) |
| | TGGACGACACCCAGTTCG | | CGACGCCGCGAGTCCGAG |
| | TG (SEQ ID NO: 238) | | AG (SEQ ID NO: 241) |
| | GCTGTCGAACCTCACGAA | | CGGCTCCTCTCTCGGACTC |
| | CT (SEQ ID NO: 239) | | G (SEQ ID NO: 242) |
| | CGCTGTCGAACCTCACGA | | CGAGTCCGAGAGAGGAG |
| | AC (SEQ ID NO: 240) | | CCG (SEQ ID NO: 243) |
| | ACAGCGACGCCACGAGTC | | GAGTCCGAGAGAGGAGC |
| | CG (SEQ ID NO: 283) | | CGC (SEQ ID NO: 244) |
| | CGACGCCACGAGTCCGAG | | GGCGCCCGCGGCTCCTCT |
| | GA (SEQ ID NO: 284) | | CT (SEQ ID NO: 245) |
| | GGGCGCCATCCTCGGACT | | GAGAGGAGCCGCGGGCG |
| | CG (SEQ ID NO: 285) | | CCG (SEQ ID NO: 246) |
| | CGAGTCCGAGGATGGCG | | TGCTCTATCCACGGCGCCC |
| | CCC (SEQ ID NO: 286) | | G (SEQ ID NO: 72) |
| | GAGTCCGAGGATGGCGC | | GCGGGCGCCGTGGATAG |
| | CCC (SEQ ID NO: 287) | | AGC (SEQ ID NO: 74) |
| | GGCGCCCGGGGCGCCATC | | GGCGCCGTGGATAGAGC |
| | CT (SEQ ID NO: 288) | | AGG (SEQ ID NO: 75) |
| | GGATGGCGCCCCGGGCG | | GCGCCGTGGATAGAGCA |
| | CCA (SEQ ID NO: 289) | | GGA (SEQ ID NO: 76) |
| | TGCTCTATCCATGGCGCCC | | CGCCGTGGATAGAGCAG |
| | G (SEQ ID NO: 290) | | GAG (SEQ ID NO: 79) |
| | CTGCTCTATCCATGGCGCC | | GGCCCCTCCTGCTCTATCC |
| | C (SEQ ID NO: 291) | | A (SEQ ID NO: 81) |
| | CCGGGCGCCATGGATAGA | | GTGGATAGAGCAGGAGG |
| | GC (SEQ ID NO: 292) | | GGC (SEQ ID NO: 83) |
| | CCTGCTCTATCCATGGCGC | | AGCAGGAGGGGCCGGAA |
| | C (SEQ ID NO: 293) | | TAT (SEQ ID NO: 308) |
| | GGCGCCATGGATAGAGC | | GCAGGAGGGGCCGGAAT |
| | AGG (SEQ ID NO: 294) | | ATT (SEQ ID NO: 309) |
| | GCGCCATGGATAGAGCA | | AGGGGCCGGAATATTGG |
| | GGA (SEQ ID NO: 295) | | GAC (SEQ ID NO: 310) |
| | CGCCATGGATAGAGCAG | | GTGTTCCGGTCCCAATATT |
| | GAG (SEQ ID NO: 296) | | C (SEQ ID NO: 311) |
| | GGCCCCTCCTGCTCTATCC | | TCTTGCAGATCTGTGTGTT |
| | A (SEQ ID NO: 81) | | C (SEQ ID NO: 312) |
| | ATGGATAGAGCAGGAGG | | CTCTCGGTCAGTCTGTGT |
| | GGC (SEQ ID NO: 297) | | GT (SEQ ID NO: 256) |
| | AGCAGGAGGGGCCGGAG | | AGACTGACCGAGAGAGCC |
| | TAT (SEQ ID NO: 85) | | TG (SEQ ID NO: 159) |
| | GCAGGAGGGGCCGGAGT | | GCAGGTTCCGCAGGCTCT |
| | ATT (SEQ ID NO: 87) | | CT (SEQ ID NO: 252) |
| | AGGGGCCGGAGTATTGG | | GAGAGCCTGCGGAACCTG |
| | GAC (SEQ ID NO: 154) | | CG (SEQ ID NO: 253) |
| | GGGGCCGGAGTATTGGG | | AGTAGCCGCGCAGGTTCC |
| | ACC (SEQ ID NO: 89) | | GC (SEQ ID NO: 254) |
| | GTCTCCCGGTCCCAATACT | | TCTGGTTGTAGTAGCCGC |
| | C (SEQ ID NO: 298) | | GC (SEQ ID NO: 114) |
| | TCTTGGAGATCTGTGTCTC | | CGGCTACTACAACCAGAG |
| | C (SEQ ID NO: 299) | | CG (SEQ ID NO: 115) |
| | GTAAGTCTGTGTGTTGGT | | |
| | CT (SEQ ID NO: 300) | | |
| | CTCTCGGTAAGTCTGTGT | | |
| | GT (SEQ ID NO: 301) | | |
| | GCGCGGTGCGCAGGTTCT | | |
| | CT (SEQ ID NO: 302) | | |
| | AGTAGCGGAGCGCGGTG | | |
| | CGC (SEQ ID NO: 303) | | |
| | CTGGTTGTAGTAGCGGAG | | |
| | CG (SEQ ID NO: 304) | | |
| | CCGCTACTACAACCAGAG | | |
| | CG (SEQ ID NO: 152) | | |
| | CCTCGCTCTGGTTGTAGTA | | |
| | G (SEQ ID NO: 153) | | |

TABLE 3C
HLA-B 15:01 and 18:01

| 15:01 | | 18:01 |
|---|---|---|
| Exon1 | Exon2 | Exon2 |
| AGCAGCAGGAGGACGGTTCG | GTAGAAATACCTCATGGAGT | AGCAGCAGGAGGAGGGTTCG |
| (SEQ ID NO: 209) | (SEQ ID NO: 134) | (SEQ ID NO: 257) |
| GAGCAGCAGGAGGACGGTTC | TGTAGAAATACCTCATGGAG | GAGCAGCAGGAGGAGGGTTC |
| (SEQ ID NO: 210) | (SEQ ID NO: 136) | (SEQ ID NO: 258) |
| AGAGCAGCAGGAGGACGGTT | GGCGGTGTAGAAATACCTCA | AGAGCAGCAGGAGGAGGGTT |
| (SEQ ID NO: 211) | (SEQ ID NO: 272) | (SEQ ID NO: 259) |
| AACCGTCCTCCTGCTGCTCT | ATTTCTACACCGCCATGTCC | GAACCCTCCTCCTGCTGCTC |
| (SEQ ID NO: 212) | (SEQ ID NO: 273) | (SEQ ID NO: 260) |
| ACCGTCCTCCTGCTGCTCTC | TACACCGCCATGTCCCGGCC | AACCCTCCTCCTGCTGCTCT |
| (SEQ ID NO: 313) | (SEQ ID NO: 274) | (SEQ ID NO: 261) |
| TCCCGAGAGCAGCAGGAGGA | GCGGCCGGGCCGGGACATG | GACCCTCCTCCTGCTGCTCTG |
| (SEQ ID NO: 314) | (SEQ ID NO: 225) | (SEQ ID NO: 262) |
| GGGCTCCCGAGAGCAGCAGG | GCCATGTCCCGGCCCGGCCG | CCCTCCTCCTGCTGCTCTGG |
| (SEQ ID NO: 315) | (SEQ ID NO: 226) | (SEQ ID NO: 263) |
| CCTGCTGCTCTCGGGAGCCC | CCATGTCCCGGCCCGGCCGC | CCCCCAGAGCAGCAGGAGGA |
| (SEQ ID NO: 316) | SEQ ID NO: 227) | (SEQ ID NO: 264) |
| CCAGGGCTCCCGAGAGCAGC | CCCGCGGCCGGGCCGGGACA | CCTCCTCCTGCTGCTCTGGG |
| (SEQ ID NO: 317) | (SEQ ID NO: 19) | (SEQ ID NO: 265) |
| CCCTGGCCCTGACCGAGACC | CATGTCCCGGCCCGGCCGCG | CCCCCCAGAGCAGCAGGAGG |
| (SEQ ID NO: 218) | (SEQ ID NO: 228) | (SEQ ID NO: 266) |
| CCAGGTCTCGGTCAGGGCCA | GGGCTCCCCGCGGCCGGGCC | CTGCCCCCCAGAGCAGCAGG |
| (SEQ ID NO: 219) | (SEQ ID NO: 25) | (SEQ ID NO: 267) |
| CCTGGCCCTGACCGAGACCT | GGGGCTCCCCGCGGCCGGGC | CCTGCTGCTCTGGGGGGCAG |
| (SEQ ID NO: 229) | (SEQ ID NO: 28) | (SEQ ID NO: 268) |
| CCCAGGTCTCGGTCAGGGCC | AAGCGGGGCTCCCCGCGGCC | CCACTGCCCCCCAGAGCAGC |
| (SEQ ID NO: 230) | (SEQ ID NO: 31) | (SEQ ID NO: 269) |
| | GAAGCGGGGCTCCCCGCGGC | CAGTGGCCCTGACCGAGACC |
| | (SEQ ID NO: 34) | (SEQ ID NO: 270) |
| | CGATGAAGCGGGGCTCCCCG | AGTGGCCCTGACCGAGACCT |
| | (SEQ ID NO: 37) | (SEQ ID NO: 271) |
| | GGAGCCCCGCTTCATCGCAG | GTGGAAATACCTCATGGAGT |
| | (SEQ ID NO: 40) | (SEQ ID NO: 1012) |
| | GAGCCCCGCTTCATCGCAGT | TGTGGAAATACCTCATGGAG |
| | (SEQ ID NO: 43) | (SEQ ID NO: 1013) |
| | TAGCCCACTGCGATGAAGCG | GGAGGTGTGGAAATACCTCA |
| | (SEQ ID NO: 47) | (SEQ ID NO: 1449) |
| | GTAGCCCACTGCGATGAAGC | ATTTCCACACCTCCGTGTCC |
| | (SEQ ID NO: 50) | (SEQ ID NO: 1450) |
| | CGTAGCCCACTGCGATGAAG | CGGGCCGGGACACGGAGGTG |
| | (SEQ ID NO: 53) | (SEQ ID NO: 1451) |
| | CTTCATCGCAGTGGGCTACG | CACACCTCCGTGTCCCGGCC |
| | (SEQ ID NO: 55) | (SEQ ID NO: 1452) |
| | TGGACGACACCCAGTTCGTG | GCGGCCGGGCCGGGACACGG |
| | (SEQ ID NO: 238) | (SEQ ID NO: 119) |
| | GCTGTCGAACCTCACGAACT | TCCGTGTCCCGGCCCGGCCG |
| | (SEQ ID NO: 239) | (SEQ ID NO: 13) |
| | CGCTGTCGAACCTCACGAAC | CCGTGTCCCGGCCCGGCCGC |
| | (SEQ ID NO: 240) | (SEQ ID NO: 16) |
| | ACAGCGACGCCGCGAGTCCG | CCCGCGGCCGGGCCGGGACA |
| | (SEQ ID NO: 318) | (SEQ ID NO: 19) |
| | CGACGCCGCGAGTCCGAGGA | CGTGTCCCGGCCCGGCCGCG |
| | (SEQ ID NO: 319) | (SEQ ID NO: 22) |
| | GGGCGCCATCCTCGGACTCG | GGGCTCCCCGCGGCCGGGCC |
| | (SEQ ID NO: 285) | (SEQ ID NO: 25) |
| | CGAGTCCGAGGATGGCGCCC | GGGGCTCCCCGCGGCCGGGC |
| | (SEQ ID NO: 286) | (SEQ ID NO: 28) |
| | GAGTCCGAGGATGGCGCCCC | AAGCGGGGCTCCCCGCGGCC |
| | (SEQ ID NO: 287) | (SEQ ID NO: 31) |
| | GGCGCCCGGGGCGCCATCCT | GAAGCGGGGCTCCCCGCGGC |
| | (SEQ ID NO: 288) | (SEQ ID NO: 34) |
| | GGATGGCGCCCCGGGCGCCA | AGATGAAGCGGGGCTCCCCG |
| | (SEQ ID NO: 289) | (SEQ ID NO: 231) |
| | TGCTCTATCCATGGCGCCCG | GGAGCCCCGCTTCATCTCAG |
| | (SEQ ID NO: 290) | (SEQ ID NO: 232) |
| | CTGCTCTATCCATGGCGCCC | GAGCCCCGCTTCATCTCAGT |
| | (SEQ ID NO: 291) | (SEQ ID NO: 233) |
| | CCGGGCGCCATGGATAGAGC | TAGCCCACTGAGATGAAGCG |
| | (SEQ ID NO: 292) | (SEQ ID NO: 234) |
| | CCTGCTCTATCCATGGCGCC | GTAGCCCACTGAGATGAAGC |
| | (SEQ ID NO: 293) | (SEQ ID NO: 235) |
| | GGCGCCATGGATAGAGCAGG | CGTAGCCCACTGAGATGAAG |
| | (SEQ ID NO: 294) | (SEQ ID NO: 236) |
| | GCGCCATGGATAGAGCAGGA | CTTCATCTCAGTGGGCTACG |
| | (SEQ ID NO: 295) | (SEQ ID NO: 237) |
| | CGCCATGGATAGAGCAGGAG | ATCTCAGTGGGCTACGTGGA |
| | (SEQ ID NO: 296) | (SEQ ID NO: 321) |
| | GGCCCCTCCTGCTCTATCCA | TGGACGGCACCCAGTTCGTG |
| | (SEQ ID NO: 81) | (SEQ ID NO: 322) |
| | ATGGATAGAGCAGGAGGGGC | GCTGTCGAACCTCACGAACT |
| | (SEQ ID NO: 297) | (SEQ ID NO: 239) |
| | AGCAGGAGGGGCCGGAGTAT | CGCTGTCGAACCTCACGAAC |
| | (SEQ ID NO: 85) | (SEQ ID NO: 240) |
| | GCAGGAGGGGCCGGAGTATT | ACAGCGACGCCGCGAGTCCG |
| | (SEQ ID NO: 87) | (SEQ ID NO: 318) |
| | AGGGGCCGGAGTATTGGGAC | CGACGCCGCGAGTCCGAGGA |
| | (SEQ ID NO: 154) | (SEQ ID NO: 319) |
| | GGGGCCGGAGTATTGGGACC | GGGCTCCGTCCTCGGACTCG |
| | (SEQ ID NO: 89) | (SEQ ID NO: 323) |
| | GTCTCCCGGTCCCAATACTC | CGAGTCCGAGGACGGAGCCC |
| | (SEQ ID NO: 298) | (SEQ ID NO: 324) |
| | TCTTGGAGATCTGTGTCTCC | GAGTCCGAGGACGGAGCCCC |
| | (SEQ ID NO: 299) | (SEQ ID NO: 325) |
| | GTAAGTCTGTGTGTTGGTCT | GGCGCCCGGGGCTCCGTCCT |
| | (SEQ ID NO: 300) | (SEQ ID NO: 326) |
| | CTCTCGGTAAGTCTGTGTGT | GGACGGAGCCCCGGGCGCCG |
| | (SEQ ID NO: 301) | (SEQ ID NO: 327) |
| | AGACTTACCGAGAGAGCCTG | TGCTCTATCCACGGCGCCCG |
| | (SEQ ID NO: 320) | (SEQ ID NO: 72) |
| | GCAGGTTCCGCAGGCTCTCT | TTGCTCTATCCACGGCGCCC |
| | (SEQ ID NO: 252) | (SEQ ID NO: 328) |
| | GAGAGCCTGCGGAACCTGCG | CTTGCTCTATCCACGGCGCC |
| | (SEQ ID NO: 253) | (SEQ ID NO: 329) |
| | AGTAGCCGCGCAGGTTCCGC | GGCGCCGTGGATAGAGCAAG |
| | (SEQ ID NO: 254) | (SEQ ID NO: 330) |
| | TCTGGTTGTAGTAGCCGCGC | GCGCCGTGGATAGAGCAAGA |
| | (SEQ ID NO: 114) | (SEQ ID NO: 331) |
| | CGGCTACTACAACCAGAGCG | CGCCGTGGATAGAGCAAGAG |
| | (SEQ ID NO: 115) | (SEQ ID NO: 332) |
| | | GGCCCCTCTTGCTCTATCCA |
| | | (SEQ ID NO: 333) |
| | | GTGGATAGAGCAAGAGGGGC |
| | | (SEQ ID NO: 334) |
| | | AGCAAGAGGGGCCGGAGTAT |
| | | (SEQ ID NO: 335) |
| | | GCAAGAGGGGCCGGAGTATT |
| | | (SEQ ID NO: 336) |
| | | AGGGGCCGGAGTATTGGGAC |
| | | (SEQ ID NO: 154) |
| | | GTGTTCCGGTCCCAATACTC |
| | | (SEQ ID NO: 155) |
| | | TCTTGGAGATCTGTGTGTTC |
| | | (SEQ ID NO: 337) |
| | | GTAAGTCTGTGTGTTGGTCT |
| | | (SEQ ID NO: 300) |
| | | CTCTCGGTAAGTCTGTGTGT |
| | | (SEQ ID NO: 301) |
| | | AGACTTACCGAGAGAGCCTG |
| | | (SEQ ID NO: 320) |
| | | GCAGGTTCCGCAGGCTCTCT |
| | | (SEQ ID NO: 252) |
| | | GAGAGCCTGCGGAACCTGCG |
| | | (SEQ ID NO: 253) |
| | | AGTAGCCGCGCAGGTTCCGC |
| | | (SEQ ID NO: 254) |
| | | TCTGGTTGTAGTAGCCGCGC |
| | | (SEQ ID NO: 114) |
| | | CGGCTACTACAACCAGAGCG |
| | | (SEQ ID NO: 115) |

TABLE 3D
HLA-B 35:01 and 38:01

| 35:01 | | 38:01 |
|---|---|---|
| CAGGACGAAGTCCCAGGCCC | AGCAGCAGGAGGACGGTTCG | AGCAGCAGGAGGACGGTTCG |
| (SEQ ID NO: 338) | (SEQ ID NO: 209) | (SEQ ID NO: 209) |
| AGGACGAAGTCCCAGGCCCC | AGCAGCAGGAGGACGGTTCG | GAGCAGCAGGAGGACGGTTC |
| (SEQ ID NO: 339) | (SEQ ID NO: 209) | (SEQ ID NO: 210) |
| ACGAAGTCCCAGGCCCCGGG | GAGCAGCAGGAGGACGGTTC | AGAGCAGCAGGAGGACGGTT |
| (SEQ ID NO: 340) | (SEQ ID NO: 210) | (SEQ ID NO: 211) |
| CGAAGTCCCAGGCCCCGGGC | GAGCAGCAGGAGGACGGTTC | AACCGTCCTCCTGCTGCTCT |
| (SEQ ID NO: 341) | (SEQ ID NO: 210) | (SEQ ID NO: 212) |
| GAAGTCCCAGGCCCCGGGCG | AGAGCAGCAGGAGGACGGTT | CGCCGAGAGCAGCAGGAGGA |
| (SEQ ID NO: 342) | (SEQ ID NO: 211) | (SEQ ID NO: 213) |
| GAGAGCCCCGCCCGGGGCCT | AGAGCAGCAGGAGGACGGTT | CGTCCTCCTGCTGCTCTCGG |
| (SEQ ID NO: 343) | (SEQ ID NO: 211) | (SEQ ID NO: 214) |
| TGAGAGCCCCGCCCGGGGCC | GAACCGTCCTCCTGCTGCTC | GGGCCGCCGAGAGCAGCAGG |
| (SEQ ID NO: 344) | (SEQ ID NO: 967) | (SEQ ID NO: 215) |
| AGGCCCCGGGCGGGGCTCTC | \|GAACCGTCCTCCTGCTGCTC | CCTGCTGCTCTCGGCGGCCC |
| (SEQ ID NO: 345) | (SEQ ID NO: 967) | (SEQ ID NO: 216) |
| GGCCCCGGGCGGGGCTCTCA | AACCGTCCTCCTGCTGCTCT | CCAGGGCCGCCGAGAGCAGC |
| (SEQ ID NO: 346) | (SEQ ID NO: 212) | (SEQ ID NO: 217) |
| GACCCTGAGAGCCCCGCCCG | AACCGTCCTCCTGCTGCTCT | CCCTGGCCCTGACCGAGACC |
| (SEQ ID NO: 347) | (SEQ ID NO: 212) | (SEQ ID NO: 218) |
| AGACCCTGAGAGCCCCGCCC | ACCGTCCTCCTGCTGCTCTG | CCAGGTCTCGGTCAGGGCCA |
| (SEQ ID NO: 348) | (SEQ ID NO: 968) | (SEQ ID NO: 219) |
| GAGACCCTGAGAGCCCCGCC | ACCGTCCTCCTGCTGCTCTG | CCTGGCCCTGACCGAGACCT |
| (SEQ ID NO: 349) | (SEQ ID NO: 968) | (SEQ ID NO: 229) |
| GGCGGGGCTCTCAGGGTCTC | CCGTCCTCCTGCTGCTCTGG | CCCAGGTCTCGGTCAGGGCC |
| (SEQ ID NO: 350) | (SEQ ID NO: 969) | (SEQ ID NO: 230) |
| TCCGAGAGCCTTGTCTGCAT | CCCCCAGAGCAGCAGGAGGA | GTAGAAATACCTCATGGAGT |
| (SEQ ID NO: 351) | (SEQ ID NO: 264) | (SEQ ID NO: 134) |
| CCGAGAGCCTTGTCTGCATT | CCGTCCTCCTGCTGCTCTGG | TGTAGAAATACCTCATGGAG |
| (SEQ ID NO: 352) | (SEQ ID NO: 969) | (SEQ ID NO: 136) |
| CCCAATGCAGACAAGGCTCT | CCCCCAGAGCAGCAGGAGGA | GGAGGTGTAGAAATACCTCA |
| (SEQ ID NO: 353) | (SEQ ID NO: 264) | (SEQ ID NO: 116) |
| CGAGAGCCTTGTCTGCATTG | CGTCCTCCTGCTGCTCTGGG | ATTTCTACACCTCCGTGTCC |
| (SEQ ID NO: 354) | (SEQ ID NO: 970) | (SEQ ID NO: 117) |
| GAGCCTTGTCTGCATTGGGG | CGTCCTCCTGCTGCTCTGGG | TACACCTCCGTGTCCCGGCC |
| (SEQ ID NO: 355) | (SEQ ID NO: 970) | (SEQ ID NO: 118) |
| GCGCCTCCCCAATGCAGACA | CTGCCCCCCAGAGCAGCAGG | GCGGCCGGGCCGGGACACGG |
| (SEQ ID NO: 356) | (SEQ ID NO: 267) | (SEQ ID NO: 119) |
| GCATTGGGGAGGCGCAGCGT | CTGCCCCCCAGAGCAGCAGG | TCCGTGTCCCGGCCCGGCCG |
| (SEQ ID NO: 357) | (SEQ ID NO: 267) | (SEQ ID NO: 13) |
| CATTGGGGAGGCGCAGCGTT | \|CCTGCTGCTCTGGGGGGCAG | CCGTGTCCCGGCCCGGCCGC |
| (SEQ ID NO: 358) | (SEQ ID NO: 268) | (SEQ ID NO: 16) |
| ATTGGGGAGGCGCAGCGTTG | CCACTGCCCCCCAGAGCAGC | CCCGCGGCCGGGCCGGGACA |
| (SEQ ID NO: 359) | (SEQ ID NO: 269) | (SEQ ID NO: 19) |
| AAGTGAAACTCGTGGGAGTG | CCTGCTGCTCTGGGGGGCAG | CGTGTCCCGGCCCGGCCGCG |
| (SEQ ID NO: 360) | (SEQ ID NO: 268) | (SEQ ID NO: 22) |
| GAAGTGAAACTCGTGGGAGT | CCACTGCCCCCCAGAGCAGC | GGGCTCCCCGCGGCCGGGCC |
| (SEQ ID NO: 361) | (SEQ ID NO: 269) | (SEQ ID NO: 25) |
| AGAAGTGAAACTCGTGGGAG | CAGTGGCCCTGACCGAGACC | GGGGCTCCCCGCGGCCGGGC |
| (SEQ ID NO: 362) | (SEQ ID NO: 270) | (SEQ ID NO: 28) |
| GGAGAAGAAGTGAAACTCGT | CAGTGGCCCTGACCGAGACC | AAGCGGGGCTCCCCGCGGCC |
| (SEQ ID NO: 363) | (SEQ ID NO: 270) | (SEQ ID NO: 31) |
| GGGAGAAGAAGTGAAACTCG | AGTGGCCCTGACCGAGACCT | GAAGCGGGGCTCCCCGCGGC |
| (SEQ ID NO: 364) | (SEQ ID NO: 271) | (SEQ ID NO: 34) |
| CTTCTTCTCCCAACCTATGT | AGTGGCCCTGACCGAGACCT | AGATGAAGCGGGGCTCCCCG |
| (SEQ ID NO: 365) | (SEQ ID NO: 271) | (SEQ ID NO: 231) |
| TTCTTCTCCCAACCTATGTC | GCCCTGACCGAGACCTGGGC | GGAGCCCCGCTTCATCTCAG |
| (SEQ ID NO: 366) | (SEQ ID NO: 971) | (SEQ ID NO: 232) |
| AGAAGGACCCGACATAGGTT | ACCGGCCCAGGTCTCGGTCA | GAGCCCCGCTTCATCTCAGT |
| (SEQ ID NO: 367) | (SEQ ID NO: 972) | (SEQ ID NO: 233) |
| AAGAAGGACCCGACATAGGT | CACCGGCCCAGGTCTCGGTC | TAGCCCACTGAGATGAAGCG |
| (SEQ ID NO: 368) | (SEQ ID NO: 973) | (SEQ ID NO: 234) |
| CTGGAAGAAGGACCCGACAT | GCACTCACCGGCCCAGGTCT | GTAGCCCACTGAGATGAAGC |
| (SEQ ID NO: 369) | (SEQ ID NO: 974) | (SEQ ID NO: 235) |
| CTATGTCGGGTCCTTCTTCC | GAGACCTGGGCCGGTGAGTG | CGTAGCCCACTGAGATGAAG |
| (SEQ ID NO: 370) | (SEQ ID NO: 975) | (SEQ ID NO: 236) |
| GTCACGAGTATCCTGGAAGA | AGACCTGGGCCGGTGAGTGC | CTTCATCTCAGTGGGCTACG |
| (SEQ ID NO: 371) | (SEQ ID NO: 976) | (SEQ ID NO: 237) |
| GGGACGCGTCACGAGTATCC | GACCTGGGCCGGTGAGTGCG | TGGACGACACGCAGTTCGTG |
| (SEQ ID NO: 372) | (SEQ ID NO: 977) | (SEQ ID NO: 57) |
| TCCCCATTTCCCACTCCCAT | GACCCCGCACTCACCGGCCC | CGACGCCGCGAGTCCGAGAG |
| (SEQ ID NO: 373) | (SEQ ID NO: 978) | (SEQ ID NO: 241) |
| CCCCATTTCCCACTCCCATT | TGGGCCGGTGAGTGCGGGGT | CGGCTCCTCTCTCGGACTCG |
| (SEQ ID NO: 374) | (SEQ ID NO: 979) | (SEQ ID NO: 242) |
| CCCAATGGGAGTGGGAAATG | GGGCCGGTGAGTGCGGGGTC | CGAGTCCGAGAGAGGAGCCG |
| (SEQ ID NO: 375) | (SEQ ID NO: 980) | (SEQ ID NO: 243) |
| ACCCAATGGGAGTGGGAAAT | CCGGTGAGTGCGGGGTCGGG | GAGTCCGAGAGAGGAGCCGC |
| (SEQ ID NO: 376) | (SEQ ID NO: 981) | (SEQ ID NO: 244) |
| CACCCAATGGGAGTGGGAAA | CCTCCCGACCCCGCACTCAC | GGCGCCCGCGGCTCCTCTCT |
| (SEQ ID NO: 377) | (SEQ ID NO: 982) | (SEQ ID NO: 245) |
| TTCCCACTCCCATTGGGTGT | CGGTGAGTGCGGGGTCGGGA | GAGAGGAGCCGCGGGCGCCG |
| (SEQ ID NO: 378) | (SEQ ID NO: 983) | (SEQ ID NO: 246) |
| ATCCGACACCCAATGGGAGT | GTGCGGGGTCGGGAGGGAAA | TGCTCTATCCACGGCGCCCG |
| (SEQ ID NO: 379) | (SEQ ID NO: 984) | (SEQ ID NO: 72) |
| TATCCGACACCCAATGGGAG | CGGGAGGGAAATGGCCTCTG | GCGGGCGCCGTGGATAGAGC |
| (SEQ ID NO: 380) | (SEQ ID NO: 985) | (SEQ ID NO: 74) |
| CTAGATATCCGACACCCAAT | GGGAGGGAAATGGCCTCTGT | GGCGCCGTGGATAGAGCAGG |
| (SEQ ID NO: 381) | (SEQ ID NO: 986) | (SEQ ID NO: 75) |
| TCTAGATATCCGACACCCAA | GGAGGGAAATGGCCTCTGTG | GCGCCGTGGATAGAGCAGGA |
| (SEQ ID NO: 382) | (SEQ ID NO: 987) | (SEQ ID NO: 76) |
| GAGAAGCCAATCAGTGTCGC | GGGAAATGGCCTCTGTGGGG | CGCCGTGGATAGAGCAGGAG |
| (SEQ ID NO: 383) | (SEQ ID NO: 988) | (SEQ ID NO: 79) |
| AGAAGCCAATCAGTGTCGCC | GGCCTCTGTGGGGAGGAGCG | GGCCCCTCCTGCTCTATCCA |
| (SEQ ID NO: 384) | (SEQ ID NO: 989) | (SEQ ID NO: 81) |
| GAAGCCAATCAGTGTCGCCG | GCCTCTGTGGGGAGGAGCGA | GTGGATAGAGCAGGAGGGGC |
| (SEQ ID NO: 385) | (SEQ ID NO: 990) | (SEQ ID NO: 83) |
| GGGACCCCGGCGACACTGAT | CCTCTGTGGGGAGGAGCGAG | AGCAGGAGGGGCCGGAATAT |
| (SEQ ID NO: 386) | (SEQ ID NO: 991) | (SEQ ID NO: 308) |
| GGACTTTAGAACTGGGACCC | CCCCTCGCTCCTCCCCACAG | GCAGGAGGGGCCGGAATATT |
| (SEQ ID NO: 387) | (SEQ ID NO: 992) | (SEQ ID NO: 309) |
| TGCGTGGGGACTTTAGAACT | GGGAGGAGCGAGGGGACCGC | AGGGGCCGGAATATTGGGAC |
| (SEQ ID NO: 388) | (SEQ ID NO: 993) | (SEQ ID NO: 310) |
| GTGCGTGGGGACTTTAGAAC | AGGAGCGAGGGGACCGCAGG | GTGTTCCGGTCCCAATATTC |
| (SEQ ID NO: 389) | (SEQ ID NO: 994) | (SEQ ID NO: 311) |
| AAAGTCCCCACGCACCCACC | GGAGCGAGGGGACCGCAGGC | TCTTGCAGATCTGTGTGTTC |
| (SEQ ID NO: 390) | (SEQ ID NO: 995) | (SEQ ID NO: 312) |
| TGAGTCCGGGTGGGTGCGTG | GAGCGAGGGGACCGCAGGCG | CTCTCGGTAAGTCTGTGTGT |
| (SEQ ID NO: 391) | (SEQ ID NO: 996) | (SEQ ID NO: 301) |
| CTGAGTCCGGGTGGGTGCGT | AGCGAGGGGACCGCAGGCGG | AGACTTACCGAGAGAACCTG |
| (SEQ ID NO: 392) | (SEQ ID NO: 997) | (SEQ ID NO: 1010) |
| TCTGAGTCCGGGTGGGTGCG | GGGACCGCAGGCGGGGGCGC | GCGCGATCCGCAGGTTCTCT |
| (SEQ ID NO: 393) | (SEQ ID NO: 998) | (SEQ ID NO: 150) |
| AGGAGATTCTGAGTCCGGGT | AGGTCCTGCGCCCCCGCCTG | AGTAGCGGAGCGCGATCCGC |
| (SEQ ID NO: 394) | (SEQ ID NO: 999) | (SEQ ID NO: 151) |
| GAGGAGATTCTGAGTCCGGG | AGGCGGGGGCGCAGGACCTG | CCGCTACTACAACCAGAGCG |
| (SEQ ID NO: 395) | (SEQ ID NO: 1000) | (SEQ ID NO: 152) |
| TCTGAGGAGATTCTGAGTCC | CAGGACCTGAGGAGCCGCGC | CCTCGCTCTGGTTGTAGTAG |
| (SEQ ID NO: 396) | (SEQ ID NO: 1001) | (SEQ ID NO: 153) |
| GTCTGAGGAGATTCTGAGTC | AGGACCTGAGGAGCCGCGCC | |
| (SEQ ID NO: 397) | (SEQ ID NO: 1002) | |
| TCTCCTCAGACGCCGAGATG | ACCTGAGGAGCCGCGCCGGG | |
| (SEQ ID NO: 398) | (SEQ ID NO: 1003) | |
| CTCCTCAGACGCCGAGATGC | TCCTCCCGGCGCGGCTCCTC | |
| (SEQ ID NO: 399) | (SEQ ID NO: 1004) | |
| GACCCGCATCTCGGCGTCTG | TGAGGAGCCGCGCCGGGAGG | |
| (SEQ ID NO: 400) | (SEQ ID NO: 1005) | |
| AGACGCCGAGATGCGGGTCA | GAGGAGCCGCGCCGGGAGGA | |
| (SEQ ID NO: 401) | (SEQ ID NO: 1006) | |
| GGGCGCCGTGACCCGCATCT | AGCCGCGCCGGGAGGAGGGT | |
| (SEQ ID NO: 402) | (SEQ ID NO: 1007) | |
| GCCGCGCCGGGAGGAGGGTC | TACCTCATGGAGTGGGAGCC | |
| (SEQ ID NO: 403) | (SEQ ID NO: 1008) | |
| GCCCGACCCTCCTCCCGGCG | GTAGAAATACCTCATGGAGT | |
| (SEQ ID NO: 404) | (SEQ ID NO: 134) | |
| GCGCCGGGAGGAGGGTCGGG | GTAGAAATACCTCATGGAGT | |
| (SEQ ID NO: 405) | (SEQ ID NO: 134) | |
| CGCCGGGAGGAGGGTCGGGC | TGTAGAAATACCTCATGGAG | |
| (SEQ ID NO: 406) | (SEQ ID NO: 136) | |
| GACCCGCCCGACCCTCCTCC | TGTAGAAATACCTCATGGAG | |
| (SEQ ID NO: 407) | (SEQ ID NO: 136) | |
| CTCAGCCCCTCCTCGCCCCC | GGCGGTGTAGAAATACCTCA | |
| (SEQ ID NO: 408) | (SEQ ID NO: 272) | |
| GGGAGCCTGGGGGCGAGGAG | GGCGGTGTAGAAATACCTCA | |
| (SEQ ID NO: 409) | (SEQ ID NO: 272) | |
| TGGGAGCCTGGGGGCGAGGA | ATTTCTACACCGCCATGTCC | |
| (SEQ ID NO: 410) | (SEQ ID NO: 273) | |
| GTGGGAGCCTGGGGGCGAGG | ATTTCTACACCGCCATGTCC | |
| (SEQ ID NO: 411) | (SEQ ID NO: 273) | |
| GGAGTGGGAGCCTGGGGGCG | TACACCGCCATGTCCCGGCC | |
| (SEQ ID NO: 412) | (SEQ ID NO: 274) | |
| CTCATGGAGTGGGAGCCTGG | TACACCGCCATGTCCCGGCC | |
| (SEQ ID NO: 413) | (SEQ ID NO: 274) | |
| CCCCAGGCTCCCACTCCATG | GCGGCCGGGCCGGGACATGG | |
| (SEQ ID NO: 414) | (SEQ ID NO: 225) | |
| CCTCATGGAGTGGGAGCCTG | GCGGCCGGGCCGGGACATGG | |
| (SEQ ID NO: 415) | (SEQ ID NO: 225) | |
| ACCTCATGGAGTGGGAGCCT | GCCATGTCCCGGCCCGGCCG | |
| (SEQ ID NO: 416) | (SEQ ID NO: 226) | |
| GCCATGTCCCGGCCCGGCCG | CGATGAAGCGGGGCTCCCCG | |
| (SEQ ID NO: 226) | (SEQ ID NO: 37) | |
| CCATGTCCCGGCCCGGCCGC | CGATGAAGCGGGGCTCCCCG | |
| (SEQ ID NO: 227) | (SEQ ID NO: 37) | |
| CCCGCGGCCGGGCCGGGACA | GGAGCCCCGCTTCATCGCAG | |
| (SEQ ID NO: 19) | (SEQ ID NO: 40) | |
| CCATGTCCCGGCCCGGCCGC | GGAGCCCCGCTTCATCGCAG | |
| (SEQ ID NO: 227) | (SEQ ID NO: 40) | |
| CCCGCGGCCGGGCCGGGACA | GAGCCCCGCTTCATCGCAGT | |
| (SEQ ID NO: 19) | (SEQ ID NO: 43) | |
| CATGTCCCGGCCCGGCCGCG | GAGCCCCGCTTCATCGCAGT | |
| (SEQ ID NO: 228) | (SEQ ID NO: 43) | |
| CATGTCCCGGCCCGGCCGCG | TAGCCCACTGCGATGAAGCG | |
| (SEQ ID NO: 228) | (SEQ ID NO: 47) | |
| GGGCTCCCCGCGGCCGGGCC | TAGCCCACTGCGATGAAGCG | |
| (SEQ ID NO: 25) | (SEQ ID NO: 47) | |
| GGGCTCCCCGCGGCCGGGCC | GTAGCCCACTGCGATGAAGC | |
| (SEQ ID NO: 25) | (SEQ ID NO: 50) | |
| GGGGCTCCCCGCGGCCGGGC | GTAGCCCACTGCGATGAAGC | |
| (SEQ ID NO: 28) | (SEQ ID NO: 50) | |
| GGGGCTCCCCGCGGCCGGGC | CGTAGCCCACTGCGATGAAG | |
| (SEQ ID NO: 28) | (SEQ ID NO: 53) | |
| AAGCGGGGCTCCCCGCGGCC | CGTAGCCCACTGCGATGAAG | |
| (SEQ ID NO: 31) | (SEQ ID NO: 53) | |
| AAGCGGGGCTCCCCGCGGCC | CTTCATCGCAGTGGGCTACG | |
| (SEQ ID NO: 31) | (SEQ ID NO: 55) | |
| GAAGCGGGGCTCCCCGCGGC | CTTCATCGCAGTGGGCTACG | |
| (SEQ ID NO: 34) | (SEQ ID NO: 55) | |
| GAAGCGGGGCTCCCCGCGGC | TGGACGACACCCAGTTCGTG | |
| (SEQ ID NO: 34) | (SEQ ID NO: 238) | |
| TGGACGACACCCAGTTCGTG | CGAGTCCGAGGACGGAGCCC | |
| (SEQ ID NO: 238) | (SEQ ID NO: 324) | |
| GCTGTCGAACCTCACGAACT | GAGTCCGAGGACGGAGCCCC | |
| (SEQ ID NO: 239) | (SEQ ID NO: 325) | |
| GCTGTCGAACCTCACGAACT | GAGTCCGAGGACGGAGCCCC | |
| (SEQ ID NO: 239) | (SEQ ID NO: 325) | |
| CGCTGTCGAACCTCACGAAC | GGCGCCCGGGGCTCCGTCCT | |
| (SEQ ID NO: 240) | (SEQ ID NO: 326) | |
| CGCTGTCGAACCTCACGAAC | GGCGCCCGGGGCTCCGTCCT | |
| (SEQ ID NO: 240) | (SEQ ID NO: 326) | |
| ACAGCGACGCCGCGAGTCCG | GGACGGAGCCCCGGGCGCCA | |
| (SEQ ID NO: 318) | (SEQ ID NO: 1009) | |
| ACAGCGACGCCGCGAGTCCG | GGACGGAGCCCCGGGCGCCA | |
| (SEQ ID NO: 318) | (SEQ ID NO: 1009) | |
| CGACGCCGCGAGTCCGAGGA | TGCTCTATCCATGGCGCCCG | |
| (SEQ ID NO: 319) | (SEQ ID NO: 290) | |
| CGACGCCGCGAGTCCGAGGA | TGCTCTATCCATGGCGCCCG | |
| (SEQ ID NO: 319) | (SEQ ID NO: 290) | |
| GGGCTCCGTCCTCGGACTCG | CTGCTCTATCCATGGCGCCC | |
| (SEQ ID NO: 323) | (SEQ ID NO: 291) | |
| GGGCTCCGTCCTCGGACTCG | CTGCTCTATCCATGGCGCCC | |
| (SEQ ID NO: 323) | (SEQ ID NO: 291) | |
| CGAGTCCGAGGACGGAGCCC | CCGGGCGCCATGGATAGAGC | |
| (SEQ ID NO: 324) | (SEQ ID NO: 292) | |
| CCTGCTCTATCCATGGCGCC | CTCTCGGTAAGTCTGTGTGT | |
| (SEQ ID NO: 293) | (SEQ ID NO: 301) | |
| CCGGGCGCCATGGATAGAGC | CTCTCGGTAAGTCTGTGTGT | |
| (SEQ ID NO: 292) | (SEQ ID NO: 301) | |
| CCTGCTCTATCCATGGCGCC | AGACTTACCGAGAGAGCCTG | |
| (SEQ ID NO: 293) | (SEQ ID NO: 320) | |
| GGCGCCATGGATAGAGCAGG | AGACTTACCGAGAGAGCCTG | |
| (SEQ ID NO: 294) | (SEQ ID NO: 320) | |
| GGCGCCATGGATAGAGCAGG | GCAGGTTCCGCAGGCTCTCT | |
| (SEQ ID NO: 294) | (SEQ ID NO: 252) | |
| GCGCCATGGATAGAGCAGGA | GCAGGTTCCGCAGGCTCTCT | |
| (SEQ ID NO: 295) | (SEQ ID NO: 252) | |
| GCGCCATGGATAGAGCAGGA | GAGAGCCTGCGGAACCTGCG | |
| (SEQ ID NO: 295) | (SEQ ID NO: 253) | |
| CGCCATGGATAGAGCAGGAG | GAGAGCCTGCGGAACCTGCG | |
| (SEQ ID NO: 296) | (SEQ ID NO: 253) | |
| CGCCATGGATAGAGCAGGAG | AGTAGCCGCGCAGGTTCCGC | |
| (SEQ ID NO: 296) | (SEQ ID NO: 254) | |
| GGCCCCTCCTGCTCTATCCA | AGTAGCCGCGCAGGTTCCGC | |
| (SEQ ID NO: 81) | (SEQ ID NO: 254) | |
| GGCCCCTCCTGCTCTATCCA | TCTGGTTGTAGTAGCCGCGC | |
| (SEQ ID NO: 81) | (SEQ ID NO: 114) | |
| ATGGATAGAGCAGGAGGGGC | TCTGGTTGTAGTAGCCGCGC | |
| (SEQ ID NO: 297) | (SEQ ID NO: 114) | |
| ATGGATAGAGCAGGAGGGGC | CGGCTACTACAACCAGAGCG | |
| (SEQ ID NO: 297) | (SEQ ID NO: 115) | |
| AGCAGGAGGGGCCGGAGTAT | CGGCTACTACAACCAGAGCG | |
| (SEQ ID NO: 85) | (SEQ ID NO: 115) | |
| AGCAGGAGGGGCCGGAGTAT | TACTACAACCAGAGCGAGGC | |
| (SEQ ID NO: 85) | (SEQ ID NO: 208) | |
| GCAGGAGGGGCCGGAGTATT | TACTACAACCAGAGCGAGGC | |
| (SEQ ID NO: 87) | (SEQ ID NO: 208) | |
| GCAGGAGGGGCCGGAGTATT | GTCACTCACCGGCCTCGCTC | |
| (SEQ ID NO: 87) | (SEQ ID NO: 679) | |
| AGGGGCCGGAGTATTGGGAC | GCGAGGCCGGTGAGTGACCC | |
| (SEQ ID NO: 154) | (SEQ ID NO: 680) | |
| AGGGGCCGGAGTATTGGGAC | GCCGGTGAGTGACCCCGGCC | |
| (SEQ ID NO: 154) | (SEQ ID NO: 681) | |
| GTGTTCCGGTCCCAATACTC | CCGGTGAGTGACCCCGGCCC | |
| (SEQ ID NO: 155) | (SEQ ID NO: 682) | |
| GTGTTCCGGTCCCAATACTC | CCCGGGCCGGGGTCACTCAC | |
| (SEQ ID NO: 155) | (SEQ ID NO: 683) | |
| TCTTGAAGATCTGTGTGTTC | CGGTGAGTGACCCCGGCCCG | |
| (SEQ ID NO: 255) | (SEQ ID NO: 684) | |
| TCTTGAAGATCTGTGTGTTC | GTGACCCCGGCCCGGGGCGC | |
| (SEQ ID NO: 255) | (SEQ ID NO: 685) | |
| GTGACCTGCGCCCCGGGCCG | ATCTCGGACCCGGAGACTCG | |
| (SEQ ID NO: 417) | (SEQ ID NO: 686) | |
| CGTGACCTGCGCCCCGGGCC | GATCTCGGACCCGGAGACTC | |
| (SEQ ID NO: 418) | (SEQ ID NO: 687) | |
| TCGTGACCTGCGCCCCGGGC | GGATCTCGGACCCGGAGACT | |
| (SEQ ID NO: 419) | (SEQ ID NO: 688) | |
| GGAGTCGTGACCTGCGCCCC | AGGGAGGCGGATCTCGGACC | |
| (SEQ ID NO: 420) | (SEQ ID NO: 689) | |
| GGGAGTCGTGACCTGCGCCC | GTCCGAGATCCGCCTCCCTG | |
| (SEQ ID NO: 421) | (SEQ ID NO: 690) | |
| GACTCCCCATCCCCCACGTA | GGCCTCAGGGAGGCGGATCT | |
| (SEQ ID NO: 422) | (SEQ ID NO: 691) | |
| CGGGCCGTACGTGGGGGATG | GATCCGCCTCCCTGAGGCCG | |
| (SEQ ID NO: 423) | (SEQ ID NO: 692) | |
| CCCATCCCCCACGTACGGCC | ATCCGCCTCCCTGAGGCCGC | |
| (SEQ ID NO: 424) | (SEQ ID NO: 693) | |
| CCGGGCCGTACGTGGGGGAT | GTCCCGCGGCCTCAGGGAGG | |
| (SEQ ID NO: 425) | (SEQ ID NO: 694) | |
| CCATCCCCCACGTACGGCCC | CGGGTCCCGCGGCCTCAGGG | |
| (SEQ ID NO: 426) | (SEQ ID NO: 695) | |
| CCCGGGCCGTACGTGGGGGA | GGGCGGGTCCCGCGGCCTCA | |
| (SEQ ID NO: 427) | (SEQ ID NO: 696) | |
| GCGACCCGGGCCGTACGTGG | TGGGCGGGTCCCGCGGCCTC | |
| (SEQ ID NO: 428) | (SEQ ID NO: 697) | |
| GGCGACCCGGGCCGTACGTG | GAGGGTCTGGGCGGGTCCCG | |
| (SEQ ID NO: 429) | (SEQ ID NO: 698) | |
| GGGCGACCCGGGCCGTACGT | GACCCGCCCAGACCCTCGAC | |
| (SEQ ID NO: 430) | (SEQ ID NO: 699) | |
| GGGGCGACCCGGGCCGTACG | CGCCGGTCGAGGGTCTGGGC | |
| (SEQ ID NO: 431) | (SEQ ID NO: 700) | |
| CCCGGGTCGCCCCGAGTCTC | TCGCCGGTCGAGGGTCTGGG | |
| (SEQ ID NO: 432) | (SEQ ID NO: 701) | |
| CCGGAGACTCGGGGCGACCC | CTCTCGCCGGTCGAGGGTCT | |
| (SEQ ID NO: 433) | (SEQ ID NO: 702) | |
| CCGGGTCGCCCCGAGTCTCC | GCTCTCGCCGGTCGAGGGTC | |
| (SEQ ID NO: 434) | (SEQ ID NO: 703) | |
| CCCGGAGACTCGGGGCGACC | CTGGGGCTCTCGCCGGTCGA | |
| (SEQ ID NO: 435) | (SEQ ID NO: 704) | |
| CCTCGACCGGCGAGAGCCCC | AGTTGAGGCCAAAATCCCCG | |
| (SEQ ID NO: 436) | (SEQ ID NO: 705) | |
| CCTGGGGCTCTCGCCGGTCG | GTTGAGGCCAAAATCCCCGC | |
| (SEQ ID NO: 437) | (SEQ ID NO: 706) | |
| AACGCGCCTGGGGCTCTCGC | AGGCCAAAATCCCCGCGGGT | |
| (SEQ ID NO: 438) | (SEQ ID NO: 707) | |
| GAGCCCCAGGCGCGTTTACC | CGACCAACCCGCGGGGATTT | |
| (SEQ ID NO: 439) | (SEQ ID NO: 708) | |
| AAACCGGGTAAACGCGCCTG | CAAAATCCCCGCGGGTTGGT | |
| (SEQ ID NO: 440) | (SEQ ID NO: 709) | |
| GAAACCGGGTAAACGCGCCT | AAAATCCCCGCGGGTTGGTC | |
| (SEQ ID NO: 441) | (SEQ ID NO: 710) | |
| TGAAACCGGGTAAACGCGCC | AAATCCCCGCGGGTTGGTCG | |
| (SEQ ID NO: 442) | (SEQ ID NO: 711) | |
| CCCGGTTTCATTTTCAGTTG | TCCCCGCGGGTTGGTCGGGG | |
| (SEQ ID NO: 443) | (SEQ ID NO: 712) | |
| CCTCAACTGAAAATGAAACC | CCCCGCGGGTTGGTCGGGGC | |
| (SEQ ID NO: 444) | (SEQ ID NO: 713) | |
| GCCTCAACTGAAAATGAAAC | CCCGCCCCGACCAACCCGCG | |
| (SEQ ID NO: 445) | (SEQ ID NO: 714) | |
| CCCGCGGGTTGGTCGGGGCG | ACGGGGCTGACCGCGGGGCC | |
| (SEQ ID NO: 446) | (SEQ ID NO: 715) | |
| CCCCGCCCCGACCAACCCGC | CGGGGCTGACCGCGGGGCCG | |
| (SEQ ID NO: 447) | (SEQ ID NO: 716) | |
| GCCCCGCCCCGACCAACCCG | CTGACCGCGGGGCCGGGGCC | |
| (SEQ ID NO: 448) | (SEQ ID NO: 717) | |
| GCGGGTTGGTCGGGGCGGGG | TGACCGCGGGGCCGGGGCCA | |
| (SEQ ID NO: 449) | (SEQ ID NO: 718) | |
| CGGGTTGGTCGGGGCGGGGC | AGACCCTGGCCCCGGCCCCG | |
| (SEQ ID NO: 450) | (SEQ ID NO: 719) | |
| GGGTTGGTCGGGGCGGGGCG | ATGATGTGAGACCCTGGCCC | |
| (SEQ ID NO: 451) | (SEQ ID NO: 720) | |
| GGTCGGGGCGGGGCGGGGCT | CTCTGGATGATGTGAGACCC | |
| (SEQ ID NO: 452) | (SEQ ID NO: 721) | |
| GTCGGGGCGGGGGGGGCTC | CAGGGTCTCACATCATCCAG | |
| (SEQ ID NO: 453) | (SEQ ID NO: 722) | |
| TCGGGGCGGGGCGGGGCTCG | CACATCATCCAGAGGATGTA | |
| (SEQ ID NO: 454) | (SEQ ID NO: 723) | |
| CGGGGCGGGGCGGGGCTCGG | GGTCGCAGCCATACATCCTC | |
| (SEQ ID NO: 455) | (SEQ ID NO: 724) | |
| GCGGGGCGGGGCTCGGGGGA | GAGGATGTATGGCTGCGACC | |
| (SEQ ID NO: 456) | (SEQ ID NO: 725) | |
| CGGGGCGGGGCTCGGGGGAC | AGGATGTATGGCTGCGACCT | |
| (SEQ ID NO: 457) | (SEQ ID NO: 726) | |
| GGGGGGGGCTCGGGGGACG | GGATGTATGGCTGCGACCTG | |
| (SEQ ID NO: 458) | (SEQ ID NO: 727) | |
| TCGGGGGACGGGGCTGACCG | GGCTGCGACCTGGGGCCCGA | |
| (SEQ ID NO: 459) | (SEQ ID NO: 728) | |
| CGGGGGACGGGGCTGACCGC | GCTGCGACCTGGGGCCCGAC | |
| (SEQ ID NO: 460) | (SEQ ID NO: 729) | |
| GGGGGACGGGGCTGACCGCG | GGAGGCGCCCGTCGGGCCCC | |
| (SEQ ID NO: 461) | (SEQ ID NO: 730) | |
| GACGGGGCTGACCGCGGGGC | CCCGACGGGCGCCTCCTCCG | |
| (SEQ ID NO: 462) | (SEQ ID NO: 731) | |
| CCGCGGAGGAGGCGCCCGTC | CCTGAGCTCCTGGACCGCGG | |
| (SEQ ID NO: 463) | (SEQ ID NO: 732) | |
| CCGACGGGCGCCTCCTCCGC | CCGCCGCGGTCCAGGAGCTC | |
| (SEQ ID NO: 464) | (SEQ ID NO: 733) | |
| CCCGCGGAGGAGGCGCCCGT | CGCGGTGTCCGCCGCGGTCC | |
| (SEQ ID NO: 465) | (SEQ ID NO: 734) | |
| ACTGGTCATGCCCGCGGAGG | CTGGACCGCGGCGGACACCG | |
| (SEQ ID NO: 466) | (SEQ ID NO: 735) | |
| CGGACTGGTCATGCCCGCGG | CTGAGCCGCGGTGTCCGCCG | |
| (SEQ ID NO: 467) | (SEQ ID NO: 736) | |
| AGGCGGACTGGTCATGCCCG | GCGCTGGGTGATCTGAGCCG | |
| (SEQ ID NO: 468) | (SEQ ID NO: 737) | |
| CATGACCAGTCCGCCTACGA | CTCAGATCACCCAGCGCAAG | |
| (SEQ ID NO: 469) | (SEQ ID NO: 738) | |
| CCAGTCCGCCTACGACGGCA | TCAGATCACCCAGCGCAAGT | |
| (SEQ ID NO: 470) | (SEQ ID NO: 739) | |
| CCTTGCCGTCGTAGGCGGAC | GATCACCCAGCGCAAGTGGG | |
| (SEQ ID NO: 471) | (SEQ ID NO: 740) | |
| GTAATCCTTGCCGTCGTAGG | CACCCAGCGCAAGTGGGAGG | |
| (SEQ ID NO: 472) | (SEQ ID NO: 741) | |
| GATGTAATCCTTGCCGTCGT | GGCCGCCTCCCACTTGCGCT | |
| (SEQ ID NO: 473) | (SEQ ID NO: 742) | |
| GGATTACATCGCCCTGAACG | GGGCCGCCTCCCACTTGCGC | |
| (SEQ ID NO: 474) | (SEQ ID NO: 743) | |
| GGAGCTCAGGTCCTCGTTCA | CAAGTGGGAGGCGGCCCGTG | |
| (SEQ ID NO: 475) | (SEQ ID NO: 744) | |
| AGGAGCTCAGGTCCTCGTTC | GTGGGAGGCGGCCCGTGTGG | |
| (SEQ ID NO: 476) | (SEQ ID NO: 745) | |
| TGAACGAGGACCTGAGCTCC | TCTCAGCTGCTCCGCCACAC | |
| (SEQ ID NO: 477) | (SEQ ID NO: 746) | |
| GGACCTGAGCTCCTGGACCG | CTCTCAGCTGCTCCGCCACA | |
| (SEQ ID NO: 478) | (SEQ ID NO: 747) | |
| GGAGCAGCTGAGAGCCTACC | CGCGCGGGTACCAGGGGCAG | |
| (SEQ ID NO: 479) | (SEQ ID NO: 748) | |
| GCAGCTGAGAGCCTACCTGG | GCGCGGGTACCAGGGGCAGT | |
| (SEQ ID NO: 480) | (SEQ ID NO: 749) | |
| CAGCTGAGAGCCTACCTGGA | CGCGGGTACCAGGGGCAGTG | |
| (SEQ ID NO: 481) | (SEQ ID NO: 750) | |
| CACGCACAGGCCCTCCAGGT | GGAAGGCTCCCCACTGCCCC | |
| (SEQ ID NO: 482) | (SEQ ID NO: 751) | |
| CTACCTGGAGGGCCTGTGCG | GAGCCTTCCCCATCTCCTAT | |
| (SEQ ID NO: 483) | (SEQ ID NO: 752) | |
| ACTCCACGCACAGGCCCTCC | CGACCTATAGGAGATGGGGA | |
| (SEQ ID NO: 484) | (SEQ ID NO: 753) | |
| TGGAGGGCCTGTGCGTGGAG | CCCCATCTCCTATAGGTCGC | |
| (SEQ ID NO: 485) | (SEQ ID NO: 754) | |
| TGCGGAGCCACTCCACGCAC | CCGGCGACCTATAGGAGATG | |
| (SEQ ID NO: 486) | (SEQ ID NO: 755) | |
| GGAGTGGCTCCGCAGATACC | CCCATCTCCTATAGGTCGCC | |
| (SEQ ID NO: 487) | (SEQ ID NO: 756) | |
| CTCCGCAGATACCTGGAGAA | CCCGGCGACCTATAGGAGAT | |
| (SEQ ID NO: 488) | (SEQ ID NO: 757) | |
| TCCGCAGATACCTGGAGAAC | CCATCTCCTATAGGTCGCCG | |
| (SEQ ID NO: 489) | (SEQ ID NO: 758) | |
| TCCCGTTCTCCAGGTATCTG | CCCCGGCGACCTATAGGAGA | |
| (SEQ ID NO: 490) | (SEQ ID NO: 759) | |
| CAGATACCTGGAGAACGGGA | CTCCTATAGGTCGCCGGGGA | |
| (SEQ ID NO: 491) | (SEQ ID NO: 760) | |
| GCGTCTCCTTCCCGTTCTCC | GGCCATCCCCGGCGACCTAT | |
| (SEQ ID NO: 492) | (SEQ ID NO: 761) | |
| GAAGGAGACGCTGCAGCGCG | TTCTCGTGGGAGGCCATCCC | |
| (SEQ ID NO: 493) | (SEQ ID NO: 762) | |
| AAGGAGACGCTGCAGCGCGC | GGATGGCCTCCCACGAGAAG | |
| (SEQ ID NO: 494) | (SEQ ID NO: 763) | |
| CGCTGCAGCGCGCGGGTACC | TGGCCTCCCACGAGAAGAGG | |
| (SEQ ID NO: 495) | (SEQ ID NO: 764) | |
| GCTGCAGCGCGCGGGTACCA | TTTCCTCCTCTTCTCGTGGG | |
| (SEQ ID NO: 496) | (SEQ ID NO: 765) | |
| CTGCAGCGCGCGGGTACCAG | CATTTTCCTCCTCTTCTCGT | |
| (SEQ ID NO: 497) | (SEQ ID NO: 766) | |
| CCACGAGAAGAGGAGGAAAA | CAGTCCCTAGAATACTGATC | |
| (SEQ ID NO: 498) | (SEQ ID NO: 767) | |
| CCATTTTCCTCCTCTTCTCG | AGTCCCTAGAATACTGATCA | |
| (SEQ ID NO: 499) | (SEQ ID NO: 768) | |
| CACGAGAAGAGGAGGAAAAT | GTCCCTAGAATACTGATCAG | |
| (SEQ ID NO: 500) | (SEQ ID NO: 769) | |
| GAATGTCGCCCTCCCTTGAA | GACCCCTGATCAGTATTCTA | |
| (SEQ ID NO: 501) | (SEQ ID NO: 770) | |
| GCCCTCCCTTGAATGGAGAA | GGACCCCTGATCAGTATTCT | |
| (SEQ ID NO: 502) | (SEQ ID NO: 771) | |
| GCCATTCTCCATTCAAGGGA | GGCTGCTGCAGGGGTCAAAG | |
| (SEQ ID NO: 503) | (SEQ ID NO: 772) | |
| TGCCATTCTCCATTCAAGGG | AGGCTGCTGCAGGGGTCAAA | |
| (SEQ ID NO: 504) | (SEQ ID NO: 773) | |
| TCATGCCATTCTCCATTCAA | AAGGCTGCTGCAGGGGTCAA | |
| (SEQ ID NO: 505) | (SEQ ID NO: 774) | |
| CTCATGCCATTCTCCATTCA | TTTGACCCCTGCAGCAGCCT | |
| (SEQ ID NO: 506) | (SEQ ID NO: 775) | |
| GTTTTCCTGAGTTTCCTCTG | TTGACCCCTGCAGCAGCCTT | |
| (SEQ ID NO: 507) | (SEQ ID NO: 776) | |
| TTTTCCTGAGTTTCCTCTGA | GGTTCCCAAGGCTGCTGCAG | |
| (SEQ ID NO: 508) | (SEQ ID NO: 777) | |
| GGGGCCCTCAGAGGAAACTC | CGGTTCCCAAGGCTGCTGCA | |
| (SEQ ID NO: 509) | (SEQ ID NO: 778) | |
| AGAGAAGAGGGGGCCCTCAG | ACGGTTCCCAAGGCTGCTGC | |
| (SEQ ID NO: 510) | (SEQ ID NO: 779) | |
| GAGGGCCCCCTCTTCTCTCT | AGGAAAAGTCACGGTTCCCA | |
| (SEQ ID NO: 511) | (SEQ ID NO: 780) | |
| ATTGTCCTAGAGAGAAGAGG | GAACCGTGACTTTTCCTCTC | |
| (SEQ ID NO: 512) | (SEQ ID NO: 781) | |
| AATTGTCCTAGAGAGAAGAG | AGGCCTGAGAGGAAAAGTCA | |
| (SEQ ID NO: 513) | (SEQ ID NO: 782) | |
| TAATTGTCCTAGAGAGAAGA | GGCAGAGAACAAGGCCTGAG | |
| (SEQ ID NO: 514) | (SEQ ID NO: 783) | |
| TTAATTGTCCTAGAGAGAAG | TGAGTGTGAGGCAGAGAACA | |
| (SEQ ID NO: 515) | (SEQ ID NO: 784) | |
| TCTTCTCTCTAGGACAATTA | TGCCTCACACTCAGTGTGTT | |
| (SEQ ID NO: 516) | (SEQ ID NO: 785) | |
| CTTCTCTCTAGGACAATTAA | GCCTCACACTCAGTGTGTTT | |
| (SEQ ID NO: 517) | (SEQ ID NO: 786) | |
| ATTAAGGGATGACGTCTCTG | CCTCACACTCAGTGTGTTTG | |
| (SEQ ID NO: 518) | (SEQ ID NO: 787) | |
| GGATGACGTCTCTGAGGAAA | CCCCAAACACACTGAGTGTG | |
| (SEQ ID NO: 519) | (SEQ ID NO: 788) | |
| TGACGTCTCTGAGGAAATGG | GTAAAGTGACTCAGAAGTGC | |
| (SEQ ID NO: 520) | (SEQ ID NO: 789) | |
| GACGTCTCTGAGGAAATGGA | ACTTTACCTCCACTCAGATC | |
| (SEQ ID NO: 521) | (SEQ ID NO: 790) | |
| ACGTCTCTGAGGAAATGGAG | TCTGCTCCTGATCTGAGTGG | |
| (SEQ ID NO: 522) | (SEQ ID NO: 791) | |
| ACTTCTGCTCCTGATCTGAG | TGACCAGCCTGAGAATGGAC | |
| (SEQ ID NO: 523) | (SEQ ID NO: 792) | |
| GAGTCTCTGAGCGGGGAACA | CCATTCTCAGGCTGGTCACA | |
| (SEQ ID NO: 524) | (SEQ ID NO: 793) | |
| CGAGTCTCTGAGCGGGGAAC | CCATGTGACCAGCCTGAGAA | |
| (SEQ ID NO: 525) | (SEQ ID NO: 794) | |
| AAAGTTCGAGTCTCTGAGCG | CATTCTCAGGCTGGTCACAT | |
| (SEQ ID NO: 526) | (SEQ ID NO: 795) | |
| GAAAGTTCGAGTCTCTGAGC | TCTCAGGCTGGTCACATGGG | |
| (SEQ ID NO: 527) | (SEQ ID NO: 796) | |
| GGAAAGTTCGAGTCTCTGAG | CTGGTCACATGGGTGGTCCT | |
| (SEQ ID NO: 528) | (SEQ ID NO: 797) | |
| ACTCGAACTTTCCAATGAAT | TGGTCACATGGGTGGTCCTA | |
| (SEQ ID NO: 529) | (SEQ ID NO: 798) | |
| TGGGATAATCTCCTATTCAT | CATCTCTCATGGGACACCCT | |
| (SEQ ID NO: 530) | (SEQ ID NO: 799) | |
| AATGAATAGGAGATTATCCC | AGGCGCTTTGCATCTCTCAT | |
| (SEQ ID NO: 531) | (SEQ ID NO: 800) | |
| TATCCCAGGTGCCTGCGTCC | CAGGCGCTTTGCATCTCTCA | |
| (SEQ ID NO: 532) | (SEQ ID NO: 801) | |
| CAGCCTGGACGCAGGCACCT | TGGGAAGAGTCAGAAAATTC | |
| (SEQ ID NO: 533) | (SEQ ID NO: 802) | |
| CCAGGTGCCTGCGTCCAGGC | GTGTCTTTGGGGGGTCTGAT | |
| (SEQ ID NO: 534) | (SEQ ID NO: 803) | |
| CCAGCCTGGACGCAGGCACC | TGTGTCTTTGGGGGGTCTGA | |
| (SEQ ID NO: 535) | (SEQ ID NO: 804) | |
| CCTGCGTCCAGGCTGGTGTC | GGGTCACGTGTGTCTTTGGG | |
| (SEQ ID NO: 536) | (SEQ ID NO: 805) | |
| CCAGACACCAGCCTGGACGC | TGGGTCACGTGTGTCTTTGG | |
| (SEQ ID NO: 537) | (SEQ ID NO: 806) | |
| CTGCGTCCAGGCTGGTGTCT | GTGGGTCACGTGTGTCTTTG | |
| (SEQ ID NO: 538) | (SEQ ID NO: 807) | |
| ACAGAACCCAGACACCAGCC | GGTGGGTCACGTGTGTCTTT | |
| (SEQ ID NO: 539) | (SEQ ID NO: 808) | |
| CTGTGCCCCTTCCCCACACC | TGGTGGGTCACGTGTGTCTT | |
| (SEQ ID NO: 540) | (SEQ ID NO: 809) | |
| GGACACCTGGTGTGGGGAAG | ATGGTCAGAGACGGGGTGGT | |
| (SEQ ID NO: 541) | (SEQ ID NO: 810) | |
| AGGACACCTGGTGTGGGGAA | CATGGTCAGAGACGGGGTGG | |
| (SEQ ID NO: 542) | (SEQ ID NO: 811) | |
| CAGGACACCTGGTGTGGGGA | CCACCCCGTCTCTGACCATG | |
| (SEQ ID NO: 543) | (SEQ ID NO: 812) | |
| TGGACAGGACACCTGGTGTG | CCTCATGGTCAGAGACGGGG | |
| (SEQ ID NO: 544) | (SEQ ID NO: 813) | |
| ATGGACAGGACACCTGGTGT | TGGCCTCATGGTCAGAGACG | |
| (SEQ ID NO: 545) | (SEQ ID NO: 814) | |
| AATGGACAGGACACCTGGTG | GTGGCCTCATGGTCAGAGAC | |
| (SEQ ID NO: 546) | (SEQ ID NO: 815) | |
| CTGAGAATGGACAGGACACC | GGTGGCCTCATGGTCAGAGA | |
| (SEQ ID NO: 547) | (SEQ ID NO: 816) | |
| CAGGTGTCCTGTCCATTCTC | CTGACCATGAGGCCACCCTG | |
| (SEQ ID NO: 548) | (SEQ ID NO: 817) | |
| TGTCCTGTCCATTCTCAGGC | AGCACCTCAGGGTGGCCTCA | |
| (SEQ ID NO: 549) | (SEQ ID NO: 818) | |
| ATGAGGCCACCCTGAGGTGC | GAGATAGAACCTTCCAGAAG | |
| (SEQ ID NO: 550) | (SEQ ID NO: 819) | |
| TGAGGCCACCCTGAGGTGCT | AGATAGAACCTTCCAGAAGT | |
| (SEQ ID NO: 551) | (SEQ ID NO: 820) | |
| CAGGGCCCAGCACCTCAGGG | CACAGCTGCCCACTTCTGGA | |
| (SEQ ID NO: 552) | (SEQ ID NO: 821) | |
| CACCCTGAGGTGCTGGGCCC | CTTCCAGAAGTGGGCAGCTG | |
| (SEQ ID NO: 553) | (SEQ ID NO: 822) | |
| ACCCTGAGGTGCTGGGCCCT | CCAGAAGTGGGCAGCTGTGG | |
| (SEQ ID NO: 554) | (SEQ ID NO: 823) | |
| GCCCAGGGCCCAGCACCTCA | CCACCACAGCTGCCCACTTC | |
| (SEQ ID NO: 555) | (SEQ ID NO: 824) | |
| AGCCCAGGGCCCAGCACCTC | GCAGCTGTGGTGGTGCCTTC | |
| (SEQ ID NO: 556) | (SEQ ID NO: 825) | |
| GGCCCTGGGCTTCTACCCTG | TATCTCTGCTCTTCTCCAGA | |
| (SEQ ID NO: 557) | (SEQ ID NO: 826) | |
| CTCCGCAGGGTAGAAGCCCA | CACATGCCATGTACAGCATG | |
| (SEQ ID NO: 558) | (SEQ ID NO: 827) | |
| TCTCCGCAGGGTAGAAGCCC | ACATGCCATGTACAGCATGA | |
| (SEQ ID NO: 559) | (SEQ ID NO: 828) | |
| AGGTCAGTGTGATCTCCGCA | CATGCCATGTACAGCATGAG | |
| (SEQ ID NO: 560) | (SEQ ID NO: 829) | |
| CAGGTCAGTGTGATCTCCGC | GCAGCCCCTCATGCTGTACA | |
| (SEQ ID NO: 561) | (SEQ ID NO: 830) | |
| CTGCGGAGATCACACTGACC | CATCTCAGGGTGAGGGGCTT | |
| (SEQ ID NO: 562) | (SEQ ID NO: 831) | |
| AGATCACACTGACCTGGCAG | CGAAGCCCCTCACCCTGAGA | |
| (SEQ ID NO: 563) | (SEQ ID NO: 832) | |
| GATCACACTGACCTGGCAGC | GAAGCCCCTCACCCTGAGAT | |
| (SEQ ID NO: 564) | (SEQ ID NO: 833) | |
| ACACTGACCTGGCAGCGGGA | AAGCCCCTCACCCTGAGATG | |
| (SEQ ID NO: 565) | (SEQ ID NO: 834) | |
| GACCTGGCAGCGGGATGGCG | TTACCCCATCTCAGGGTGAG | |
| (SEQ ID NO: 566) | (SEQ ID NO: 835) | |
| GTCCTCGCCATCCCGCTGCC | CTTACCCCATCTCAGGGTGA | |
| (SEQ ID NO: 567) | (SEQ ID NO: 836) | |
| GGATGGCGAGGACCAAACTC | CCTCACCCTGAGATGGGGTA | |
| (SEQ ID NO: 568) | (SEQ ID NO: 837) | |
| CAAGCTCAGTGTCCTGAGTT | CCTTACCCCATCTCAGGGTG | |
| (SEQ ID NO: 569) | (SEQ ID NO: 838) | |
| AACTCAGGACACTGAGCTTG | CACCCTGAGATGGGGTAAGG | |
| (SEQ ID NO: 570) | (SEQ ID NO: 839) | |
| CTTGTGGAGACCAGACCAGC | ACCCTGAGATGGGGTAAGGA | |
| (SEQ ID NO: 571) | (SEQ ID NO: 840) | |
| GGTTCTATCTCCTGCTGGTC | CCCTGAGATGGGGTAAGGAG | |
| (SEQ ID NO: 572) | (SEQ ID NO: 841) | |
| TGGAAGGTTCTATCTCCTGC | CCCCTCCTTACCCCATCTCA | |
| (SEQ ID NO: 573) | (SEQ ID NO: 842) | |
| CCTGAGATGGGGTAAGGAGG | CCAGTCCACCATCCCCATCG | |
| (SEQ ID NO: 574) | (SEQ ID NO: 843) | |
| CCCCCTCCTTACCCCATCTC | CCACGATGGGGATGGTGGAC | |
| (SEQ ID NO: 575) | (SEQ ID NO: 844) | |
| ATGGGGTAAGGAGGGGGATG | CAGTCCACCATCCCCATCGT | |
| (SEQ ID NO: 576) | (SEQ ID NO: 845) | |
| TGGGGTAAGGAGGGGGATGA | AATGCCCACGATGGGGATGG | |
| (SEQ ID NO: 577) | (SEQ ID NO: 846) | |
| GGGGTAAGGAGGGGGATGAG | AACAATGCCCACGATGGGGA | |
| (SEQ ID NO: 578) | (SEQ ID NO: 847) | |
| GAGGGGTCATATCTCTTCTC | CAGCAACAATGCCCACGATG | |
| (SEQ ID NO: 579) | (SEQ ID NO: 848) | |
| AGGGGTCATATCTCTTCTCA | CCCATCGTGGGCATTGTTGC | |
| (SEQ ID NO: 580) | (SEQ ID NO: 849) | |
| TATCTCTTCTCAGGGAAAGC | CCAGCAACAATGCCCACGAT | |
| (SEQ ID NO: 581) | (SEQ ID NO: 850) | |
| AGGGAAAGCAGGAGCCCTTC | GCCAGCAACAATGCCCACGA | |
| (SEQ ID NO: 582) | (SEQ ID NO: 851) | |
| CCCTTCTGGAGCCCTTCAGC | CGTGGGCATTGTTGCTGGCC | |
| (SEQ ID NO: 583) | (SEQ ID NO: 852) | |
| CCTGCTGAAGGGCTCCAGAA | CCTGGCTGTCCTAGCAGTTG | |
| (SEQ ID NO: 584) | (SEQ ID NO: 853) | |
| CCTTCTGGAGCCCTTCAGCA | CCACAACTGCTAGGACAGCC | |
| (SEQ ID NO: 585) | (SEQ ID NO: 854) | |
| CCCTGCTGAAGGGCTCCAGA | GTCCTAGCAGTTGTGGTCAT | |
| (SEQ ID NO: 586) | (SEQ ID NO: 855) | |
| TGGAGCCCTTCAGCAGGGTC | CTCCGATGACCACAACTGCT | |
| (SEQ ID NO: 587) | (SEQ ID NO: 856) | |
| GGAGCCCTTCAGCAGGGTCA | AGTTGTGGTCATCGGAGCTG | |
| (SEQ ID NO: 588) | (SEQ ID NO: 857) | |
| GGGGCCCTGACCCTGCTGAA | TGGTCGCTACTGTGATGTGT | |
| (SEQ ID NO: 589) | (SEQ ID NO: 858) | |
| AGGGGCCCTGACCCTGCTGA | TCGCTACTGTGATGTGTAGG | |
| (SEQ ID NO: 590) | (SEQ ID NO: 859) | |
| GGAAAGGAGGGGAAGACGAG | ATGTGTAGGAGGAAGAGCTC | |
| (SEQ ID NO: 591) | (SEQ ID NO: 860) | |
| GGGAAAGGAGGGGAAGACGA | GTAGGAGGAAGAGCTCAGGT | |
| (SEQ ID NO: 592) | (SEQ ID NO: 861) | |
| TGGGAAAGGAGGGGAAGACG | TAGGAGGAAGAGCTCAGGTA | |
| (SEQ ID NO: 593) | (SEQ ID NO: 862) | |
| AGATGGCTCTGGGAAAGGAG | AGGAAGAGCTCAGGTAGGGA | |
| (SEQ ID NO: 594) | (SEQ ID NO: 863) | |
| AAGATGGCTCTGGGAAAGGA | GGAAGAGCTCAGGTAGGGAA | |
| (SEQ ID NO: 595) | (SEQ ID NO: 864) | |
| GAAGATGGCTCTGGGAAAGG | GAAGAGCTCAGGTAGGGAAG | |
| (SEQ ID NO: 596) | (SEQ ID NO: 865) | |
| TGGGAAGATGGCTCTGGGAA | GCTCAGGTAGGGAAGGGGTG | |
| (SEQ ID NO: 597) | (SEQ ID NO: 866) | |
| TGGACTGGGAAGATGGCTCT | CTCAGGTAGGGAAGGGGTGA | |
| (SEQ ID NO: 598) | (SEQ ID NO: 867) | |
| GTGGACTGGGAAGATGGCTC | TCAGGTAGGGAAGGGGTGAG | |
| (SEQ ID NO: 599) | (SEQ ID NO: 868) | |
| GGGATGGTGGACTGGGAAGA | GGTAGGGAAGGGGTGAGGGG | |
| (SEQ ID NO: 600) | (SEQ ID NO: 869) | |
| CACGATGGGGATGGTGGACT | GTAGGGAAGGGGTGAGGGGT | |
| (SEQ ID NO: 601) | (SEQ ID NO: 870) | |
| TAGGGAAGGGGTGAGGGGTG | CTGCACAAAAGAGTAAGTGC | |
| (SEQ ID NO: 602) | (SEQ ID NO: 871) | |
| AAGGGGTGAGGGGGGGGTC | TGCAGCACATGTGACAATGA | |
| (SEQ ID NO: 603) | (SEQ ID NO: 872) | |
| AGGGGTGAGGGGTGGGGTCT | GCACATGTGACAATGAAGGA | |
| (SEQ ID NO: 604) | (SEQ ID NO: 873) | |
| TCTGGGTTTTCTTGTCCCAC | GGACGGATGTATCACCTTGA | |
| (SEQ ID NO: 605) | (SEQ ID NO: 874) | |
| CTGGGTTTTCTTGTCCCACT | ATGTATCACCTTGATGGTTG | |
| (SEQ ID NO: 606) | (SEQ ID NO: 875) | |
| TGGGTTTTCTTGTCCCACTG | CACCTTGATGGTTGTGGTGT | |
| (SEQ ID NO: 607) | (SEQ ID NO: 876) | |
| GGGTTTTCTTGTCCCACTGG | ACCTTGATGGTTGTGGTGTT | |
| (SEQ ID NO: 608) | (SEQ ID NO: 877) | |
| TGGGGCTTGAAACCCCCAGT | CCTTGATGGTTGTGGTGTTG | |
| (SEQ ID NO: 609) | (SEQ ID NO: 878) | |
| CTGGGGCTTGAAACCCCCAG | CCCCAACACCACAACCATCA | |
| (SEQ ID NO: 610) | (SEQ ID NO: 879) | |
| CACTGGGGGTTTCAAGCCCC | GACTCATGAATGCTGAAATC | |
| (SEQ ID NO: 611) | (SEQ ID NO: 880) | |
| GCAGGGAACACTTCTACCTG | GATTTCAGCATTCATGAGTC | |
| (SEQ ID NO: 612) | (SEQ ID NO: 881) | |
| GGCAGGGAACACTTCTACCT | ATTTCAGCATTCATGAGTCA | |
| (SEQ ID NO: 613) | (SEQ ID NO: 882) | |
| AGGCAGGGAACACTTCTACC | TTTCAGCATTCATGAGTCAG | |
| (SEQ ID NO: 614) | (SEQ ID NO: 883) | |
| AGTGTTCCCTGCCTCATTAC | AGCATTCATGAGTCAGGGGA | |
| (SEQ ID NO: 615) | (SEQ ID NO: 884) | |
| GTGTTCCCTGCCTCATTACT | TCAGGGGAAGGTCCCTGCTA | |
| (SEQ ID NO: 616) | (SEQ ID NO: 885) | |
| TGCTTCCCAGTAATGAGGCA | CCCTGCTAAGGACAGACCTT | |
| (SEQ ID NO: 617) | (SEQ ID NO: 886) | |
| CTGCTTCCCAGTAATGAGGC | CCTAAGGTCTGTCCTTAGCA | |
| (SEQ ID NO: 618) | (SEQ ID NO: 887) | |
| GATGCTGCTTCCCAGTAATG | TCCTAAGGTCTGTCCTTAGC | |
| (SEQ ID NO: 619) | (SEQ ID NO: 888) | |
| CTGGGAAGCAGCATCCACAC | TGCTAAGGACAGACCTTAGG | |
| (SEQ ID NO: 620) | (SEQ ID NO: 889) | |
| TGGGAAGCAGCATCCACACA | GCTAAGGACAGACCTTAGGA | |
| (SEQ ID NO: 621) | (SEQ ID NO: 890) | |
| GGGAAGCAGCATCCACACAG | ACAGACCTTAGGAGGGCAGT | |
| (SEQ ID NO: 622) | (SEQ ID NO: 891) | |
| AGGCTGCGTTAGCCCCTGTG | CTGGACCAACTGCCCTCCTA | |
| (SEQ ID NO: 623) | (SEQ ID NO: 892) | |
| ACACAGGGGCTAACGCAGCC | CTTAGGAGGGCAGTTGGTCC | |
| (SEQ ID NO: 624) | (SEQ ID NO: 893) | |
| CACAGGGGCTAACGCAGCCT | AGGAAAGCAAGTGTGGGTCC | |
| (SEQ ID NO: 625) | (SEQ ID NO: 894) | |
| GTGCTGGCACACAGGGTCCC | AACACGAGGAAAGCAAGTGT | |
| (SEQ ID NO: 626) | (SEQ ID NO: 895) | |
| AGAGTAAGTGCTGGCACACA | AAACACGAGGAAAGCAAGTG | |
| (SEQ ID NO: 627) | (SEQ ID NO: 896) | |
| AAGAGTAAGTGCTGGCACAC | AGGCAGGATCAGGAAACACG | |
| (SEQ ID NO: 628) | (SEQ ID NO: 897) | |
| CGTGTTTCCTGATCCTGCCT | TTTCTTCCCACAGGTGGAAA | |
| (SEQ ID NO: 629) | (SEQ ID NO: 898) | |
| GTGTTTCCTGATCCTGCCTT | CTTCCCACAGGTGGAAAAGG | |
| (SEQ ID NO: 630) | (SEQ ID NO: 899) | |
| TACAGACCCAAGGCAGGATC | TTCCCACAGGTGGAAAAGGA | |
| (SEQ ID NO: 631) | (SEQ ID NO: 900) | |
| TATGACTACAGACCCAAGGC | CTCCCTCCTTITCCACCTGT | |
| (SEQ ID NO: 632) | (SEQ ID NO: 901) | |
| GAAGTATGACTACAGACCCA | GCTCCCTCCTTTTCCACCTG | |
| (SEQ ID NO: 633) | (SEQ ID NO: 902) | |
| TGGGTCTGTAGTCATACTTC | AAAAGGAGGGAGCTACTCTC | |
| (SEQ ID NO: 634) | (SEQ ID NO: 903) | |
| ATACTTCTGGAAATTCCTTT | ACTCTCAGGCTGCGTGTAAG | |
| (SEQ ID NO: 635) | (SEQ ID NO: 904) | |
| TACTTCTGGAAATTCCTTTT | CTCAGGCTGCGTGTAAGTGG | |
| (SEQ ID NO: 636) | (SEQ ID NO: 905) | |
| TTCCTTTTGGGTCCAAGACG | TCAGGCTGCGTGTAAGTGGT | |
| (SEQ ID NO: 637) | (SEQ ID NO: 906) | |
| CTCCTCGTCTTGGACCCAAA | CAGGCTGCGTGTAAGTGGTG | |
| (SEQ ID NO: 638) | (SEQ ID NO: 907) | |
| CTTTTGGGTCCAAGACGAGG | AGGCTGCGTGTAAGTGGTGG | |
| (SEQ ID NO: 639) | (SEQ ID NO: 908) | |
| TTAGAGGAACCTCCTCGTCT | CTGCGTGTAAGTGGTGGGGG | |
| (SEQ ID NO: 640) | (SEQ ID NO: 909) | |
| GAGGTTCCTCTAAGATCTCA | TGCGTGTAAGTGGTGGGGGT | |
| (SEQ ID NO: 641) | (SEQ ID NO: 910) | |
| GCAGGGCCATGAGATCTTAG | AAGTGGTGGGGGTGGGAGTG | |
| (SEQ ID NO: 642) | (SEQ ID NO: 911) | |
| GAGGGGACTGGGAGGAAGCA | TGGTGGGGGTGGGAGTGTGG | |
| (SEQ ID NO: 643) | (SEQ ID NO: 912) | |
| TGAGGGGACTGGGAGGAAGC | GACAGGAGGAATTATGGGGT | |
| (SEQ ID NO: 644) | (SEQ ID NO: 913) | |
| CTTCCTCCCAGTCCCCTCAC | GGACAGGAGGAATTATGGGG | |
| (SEQ ID NO: 645) | (SEQ ID NO: 914) | |
| TGTCCTGTGAGGGGACTGGG | GTGGGACAGGAGGAATTATG | |
| (SEQ ID NO: 646) | (SEQ ID NO: 915) | |
| AAATGTCCTGTGAGGGGACT | CGTGGGACAGGAGGAATTAT | |
| (SEQ ID NO: 647) | (SEQ ID NO: 916) | |
| AAAATGTCCTGTGAGGGGAC | ACGTGGGACAGGAGGAATTA | |
| (SEQ ID NO: 648) | (SEQ ID NO: 917) | |
| GGAAGAAAATGTCCTGTGAG | CGCAGGAGACGTGGGACAGG | |
| (SEQ ID NO: 649) | (SEQ ID NO: 918) | |
| GGGAAGAAAATGTCCTGTGA | CTCCTGTCCCACGTCTCCTG | |
| (SEQ ID NO: 650) | (SEQ ID NO: 919) | |
| TGGGAAGAAAATGTCCTGTG | TCCTGTCCCACGTCTCCTGC | |
| (SEQ ID NO: 651) | (SEQ ID NO: 920) | |
| ACAGGACATTTTCTTCCCAC | GCCCGCAGGAGACGTGGGAC | |
| (SEQ ID NO: 652) | (SEQ ID NO: 921) | |
| GGACATTTTCTTCCCACAGG | TCAGAGCCCGCAGGAGACGT | |
| (SEQ ID NO: 653) | (SEQ ID NO: 922) | |
| GTCAGAGCCCGCAGGAGACG | CTAGAGTGGGCGGGGGGGGC | |
| (SEQ ID NO: 654) | (SEQ ID NO: 923) | |
| GTCTCCTGCGGGCTCTGACC | TAGAGTGGGCGGGGGGGGCG | |
| (SEQ ID NO: 655) | (SEQ ID NO: 924) | |
| AGGACCTGGTCAGAGCCCGC | AGTGGGCGGGGGGGGCGGGG | |
| (SEQ ID NO: 656) | (SEQ ID NO: 925) | |
| AGTAGAACAAAAACAGGACC | GTGGGCGGGGGGGGCGGGGA | |
| (SEQ ID NO: 657) | (SEQ ID NO: 926) | |
| GGCTGGAGTAGAACAAAAAC | TGGGCGGGGGGGGCGGGGAG | |
| (SEQ ID NO: 658) | (SEQ ID NO: 927) | |
| TCCAGCCAGCGACAGTGCCC | GGGCGGGGGGGGCGGGGAGG | |
| (SEQ ID NO: 659) | (SEQ ID NO: 928) | |
| CCAGCCAGCGACAGTGCCCA | GGGGGGCGGGGAGGGGGCAG | |
| (SEQ ID NO: 660) | (SEQ ID NO: 929) | |
| CCCTGGGCACTGTCGCTGGC | GGGGGCGGGGAGGGGGCAGA | |
| (SEQ ID NO: 661) | (SEQ ID NO: 930) | |
| AGAGCCCTGGGCACTGTCGC | GGGGCGGGGAGGGGGCAGAG | |
| (SEQ ID NO: 662) | (SEQ ID NO: 931) | |
| GAGAGACACATCAGAGCCCT | GGGGAGGGGGCAGAGGGGAA | |
| (SEQ ID NO: 663) | (SEQ ID NO: 932) | |
| TGAGAGACACATCAGAGCCC | GGGGGCAGAGGGGAAAGGCC | |
| (SEQ ID NO: 664) | (SEQ ID NO: 933) | |
| GTGTCTCTCACAGCTTGAAA | GGGGCAGAGGGGAAAGGCCT | |
| (SEQ ID NO: 665) | (SEQ ID NO: 934) | |
| GCTTGAAAAGGTGAGATTCT | GAGGGGAAAGGCCTGGGTAA | |
| (SEQ ID NO: 666) | (SEQ ID NO: 935) | |
| CTTGAAAAGGTGAGATTCTT | TCAAAGAATCTCCATTACCC | |
| (SEQ ID NO: 667) | (SEQ ID NO: 936) | |
| TTGAAAAGGTGAGATTCTTG | GGTAATGGAGATTCTTTGAT | |
| (SEQ ID NO: 668) | (SEQ ID NO: 938) | |
| GAGATTCTTGGGGTCTAGAG | GTAATGGAGATTCTTTGATT | |
| (SEQ ID NO: 669) | (SEQ ID NO: 939) | |
| AGATTCTTGGGGTCTAGAGT | GGGATGTTTCGCGTGTGTCG | |
| (SEQ ID NO: 670) | (SEQ ID NO: 940) | |
| TTCTTGGGGTCTAGAGTGGG | GGATGTTTCGCGTGTGTCGT | |
| (SEQ ID NO: 671) | (SEQ ID NO: 941) | |
| TCTTGGGGTCTAGAGTGGGC | GAACAAATTCTGGTTAGTCA | |
| (SEQ ID NO: 672) | (SEQ ID NO: 942) | |
| CTTGGGGTCTAGAGTGGGCG | CAACAGTCATGAACAAATTC | |
| (SEQ ID NO: 673) | (SEQ ID NO: 943) | |
| TTGGGGTCTAGAGTGGGCGG | CCTGAGACAGCTGTCTTGTG | |
| (SEQ ID NO: 674) | (SEQ ID NO: 944) | |
| TGGGGTCTAGAGTGGGCGGG | CCTCACAAGACAGCTGTCTC | |
| (SEQ ID NO: 675) | (SEQ ID NO: 945) | |
| GGGGTCTAGAGTGGGCGGGG | CTGAGACAGCTGTCTTGTGA | |
| (SEQ ID NO: 676) | (SEQ ID NO: 946) | |
| GGGTCTAGAGTGGGCGGGGG | CTTGTGAGGGACTGAGATGC | |
| (SEQ ID NO: 677) | (SEQ ID NO: 947) | |
| TCTAGAGTGGGCGGGGGGGG | TCCTCCCCTTTGTGACTTCA | |
| (SEQ ID NO: 678) | (SEQ ID NO: 948) | |
| | CCTCCCCTTTGTGACTTCAA | |
| | (SEQ ID NO: 949) | |
| | CCCTTGAAGTCACAAAGGGG | |
| | (SEQ ID NO: 950) | |
| | AGGCCCTTGAAGTCACAAAG | |
| | (SEQ ID NO: 951) | |
| | GAGGCCCTTGAAGTCACAAA | |
| | (SEQ ID NO: 952) | |
| | AGAGGCCCTTGAAGTCACAA | |
| | (SEQ ID NO: 953) | |
| | TTTGTGACTTCAAGGGCCTC | |
| | (SEQ ID NO: 954) | |
| | TTTGCAGAAAGAGATGCCAG | |
| | (SEQ ID NO: 955) | |
| | TCTGGCATCTCTTTCTGCAA | |
| | (SEQ ID NO: 956) | |
| | ACAGGGACGCAGACACATTC | |
| | (SEQ ID NO: 957) | |
| | GTCCCTGTTAGCCTAATGTG | |
| | (SEQ ID NO: 958) | |
| | CTCCTCACATTAGGCTAACA | |
| | (SEQ ID NO: 959) | |
| | CCTGTTAGCCTAATGTGAGG | |
| | (SEQ ID NO: 960) | |
| | CCTCCTCACATTAGGCTAAC | |
| | (SEQ ID NO: 961) | |
| | GTTAGCCTAATGTGAGGAGG | |
| | (SEQ ID NO: 962) | |
| | TCTCTCCACCTCCTCACATT | |
| | (SEQ ID NO: 963) | |
| | GTCACAGTGGACACGGGGGT | |
| | (SEQ ID NO: 964) | |
| | GGTCACAGTGGACACGGGGG | |
| | (SEQ ID NO: 965) | |
| | AGGGGTCACAGTGGACACGG | |
| | (SEQ ID NO: 966) | |

TABLE 3E
HLA-B 40:01 and 44:02

| 40:01 | 44:02 |
|---|---|
| Exon1 | |
| AGCAGCAGGAGGACGGTTCG (SEQ ID NO: 209) | AGCAGCAGGAGGAGGGTTCG (SEQ ID NO: 257) |
| GAGCAGCAGGAGGACGGTTC (SEQ ID NO: 210) | GAGCAGCAGGAGGAGGGTTC (SEQ ID NO: 258) |
| AGAGCAGCAGGAGGACGGTT (SEQ ID NO: 211) | AGAGCAGCAGGAGGAGGGTT (SEQ ID NO: 259) |
| AACCGTCCTCCTGCTGCTCT (SEQ ID NO: 212) | GAACCCTCCTCCTGCTGCTC (SEQ ID NO: 260) |
| CGCCGAGAGCAGCAGGAGGA (SEQ ID NO: 213) | AACCCTCCTCCTGCTGCTCT (SEQ ID NO: 261) |
| CGTCCTCCTGCTGCTCTCGG (SEQ ID NO: 214) | ACCCTCCTCCTGCTGCTCTG (SEQ ID NO: 262) |
| GGGCCGCCGAGAGCAGCAGG (SEQ ID NO: 215) | CCCTCCTCCTGCTGCTCTGG (SEQ ID NO: 263) |
| CCTGCTGCTCTCGGCGGCCC (SEQ ID NO: 216) | CCCCCAGAGCAGCAGGAGGA (SEQ ID NO: 264) |
| CCAGGGCCGCCGAGAGCAGC (SEQ ID NO: 217) | CCTCCTCCTGCTGCTCTGGG (SEQ ID NO: 265) |
| CCCTGGCCCTGACCGAGACC (SEQ ID NO: 218) | CCCCCCAGAGCAGCAGGAGG (SEQ ID NO: 266) |
| CCAGGTCTCGGTCAGGGCCA (SEQ ID NO: 219) | CTGCCCCCCAGAGCAGCAGG (SEQ ID NO: 267) |
| CCTGGCCCTGACCGAGACCT (SEQ ID NO: 229) | CCTGCTGCTCTGGGGGGCAG (SEQ ID NO: 268) |
| CCCAGGTCTCGGTCAGGGCC (SEQ ID NO: 230) | CCACTGCCCCCCAGAGCAGC (SEQ ID NO: 269) |
| GGCCCTGACCGAGACCTGGG (SEQ ID NO: 1011) | CAGTGGCCCTGACCGAGACC (SEQ ID NO: 270) |
| GCCCTGACCGAGACCTGGGC (SEQ ID NO: 971) | AGTGGCCCTGACCGAGACCT (SEQ ID NO: 271) |
| GTGGAAATACCTCATGGAGT (SEQ ID NO: 1012) | GTAGAAATACCTCATGGAGT (SEQ ID NO: 134) |
| TGTGGAAATACCTCATGGAG (SEQ ID NO: 1013) | TGTAGAAATACCTCATGGAG (SEQ ID NO: 136) |
| GGCGGTGTGGAAATACCTCA (SEQ ID NO: 1014) | GGCGGTGTAGAAATACCTCA (SEQ ID NO: 272) |
| ATTTCCACACCGCCATGTCC (SEQ ID NO: 1015) | ATTTCTACACCGCCATGTCC (SEQ ID NO: 273) |
| CGGGCCGGGACATGGCGGTG (SEQ ID NO: 1016) | TACACCGCCATGTCCCGGCC (SEQ ID NO: 274) |
| CACACCGCCATGTCCCGGCC (SEQ ID NO: 1017) | GCGGCCGGGCCGGGACATGG (SEQ ID NO: 225) |
| GCGGCCGGGCCGGGACATGG (SEQ ID NO: 225) | GCCATGTCCCGGCCCGGCCG (SEQ ID NO: 226) |
| GCCATGTCCCGGCCCGGCCG (SEQ ID NO: 226) | CCATGTCCCGGCCCGGCCGC (SEQ ID NO: 227) |
| CCATGTCCCGGCCCGGCCGC (SEQ ID NO: 227) | CCCGCGGCCGGGCCGGGACA (SEQ ID NO: 19) |
| CCCGCGGCCGGGCCGGGACA (SEQ ID NO: 19) | CATGTCCCGGCCCGGCCGCG (SEQ ID NO: 228) |
| CATGTCCCGGCCCGGCCGCG (SEQ ID NO: 228) | GGGCTCCCCGCGGCCGGGCC (SEQ ID NO: 25) |
| GGGCTCCCCGCGGCCGGGCC (SEQ ID NO: 25) | GGGGCTCCCCGCGGCCGGGC (SEQ ID NO: 28) |
| GGGGCTCCCCGCGGCCGGGC (SEQ ID NO: 28) | AAGCGGGGCTCCCCGCGGCC (SEQ ID NO: 31) |
| AAGCGGGGCTCCCCGCGGCC (SEQ ID NO: 31) | GAAGCGGGGCTCCCCGCGGC (SEQ ID NO: 34) |
| GAAGCGGGGCTCCCCGCGGC (SEQ ID NO: 34) | TGATGAAGCGGGGCTCCCCG (SEQ ID NO: 275) |
| TGATGAAGCGGGGCTCCCCG (SEQ ID NO: 275) | GGAGCCCCGCTTCATCACCG (SEQ ID NO: 276) |
| GGAGCCCCGCTTCATCACCG (SEQ ID NO: 276) | GAGCCCCGCTTCATCACCGT (SEQ ID NO: 277) |
| GAGCCCCGCTTCATCACCGT (SEQ ID NO: 277) | TAGCCCACGGTGATGAAGCG (SEQ ID NO: 278) |
| TAGCCCACGGTGATGAAGCG (SEQ ID NO: 278) | GTAGCCCACGGTGATGAAGC (SEQ ID NO: 279) |
| GTAGCCCACGGTGATGAAGC (SEQ ID NO: 279) | CGTAGCCCACGGTGATGAAG (SEQ ID NO: 280) |
| CGTAGCCCACGGTGATGAAG (SEQ ID NO: 280) | CTTCATCACCGTGGGCTACG (SEQ ID NO: 281) |
| CTTCATCACCGTGGGCTACG (SEQ ID NO: 281) | CGTGTCGTCCACGTAGCCCA (SEQ ID NO: 60) |
| CGTGTCGTCCACGTAGCCCA (SEQ ID NO: 60) | TGGACGACACGCTGTTCGTG (SEQ ID NO: 1018) |
| TGGACGACACGCTGTTCGTG (SEQ ID NO: 1018) | ACAGCGACGCCACGAGTCCG (SEQ ID NO: 283) |
| ACAGCGACGCCACGAGTCCG (SEQ ID NO: 283) | CGACGCCACGAGTCCGAGGA (SEQ ID NO: 284) |
| CGACGCCACGAGTCCGAGGA (SEQ ID NO: 284) | CGGCTCCTTCCTCGGACTCG (SEQ ID NO: 1019) |
| CGGCTCCTTCCTCGGACTCG (SEQ ID NO: 1019) | CGAGTCCGAGGAAGGAGCCG (SEQ ID NO: 1020) |
| CGAGTCCGAGGAAGGAGCCG (SEQ ID NO: 1020) | GAGTCCGAGGAAGGAGCCGC (SEQ ID NO: 1021) |
| GAGTCCGAGGAAGGAGCCGC (SEQ ID NO: 1021) | GGCGCCCGCGGCTCCTTCCT (SEQ ID NO: 1022) |
| GGCGCCCGCGGCTCCTTCCT (SEQ ID NO: 1022) | GGAAGGAGCCGCGGGCGCCA (SEQ ID NO: 1023) |
| GGAAGGAGCCGCGGGCGCCA (SEQ ID NO: 1023) | TGCTCTATCCATGGCGCCCG (SEQ ID NO: 290) |
| TGCTCTATCCATGGCGCCCG (SEQ ID NO: 290) | GCGGGCGCCATGGATAGAGC (SEQ ID NO: 1024) |
| GCGGGCGCCATGGATAGAGC (SEQ ID NO: 1024) | GGCGCCATGGATAGAGCAGG (SEQ ID NO: 294) |
| GGCGCCATGGATAGAGCAGG (SEQ ID NO: 294) | GCGCCATGGATAGAGCAGGA (SEQ ID NO: 295) |
| GCGCCATGGATAGAGCAGGA (SEQ ID NO: 295) | CGCCATGGATAGAGCAGGAG (SEQ ID NO: 296) |
| CGCCATGGATAGAGCAGGAG (SEQ ID NO: 296) | GGCCCCTCCTGCTCTATCCA (SEQ ID NO: 81) |
| GGCCCCTCCTGCTCTATCCA (SEQ ID NO: 81) | ATGGATAGAGCAGGAGGGGC (SEQ ID NO: 297) |
| ATGGATAGAGCAGGAGGGGC (SEQ ID NO: 297) | AGCAGGAGGGGCCGGAGTAT (SEQ ID NO: 85) |
| AGCAGGAGGGGCCGGAGTAT (SEQ ID NO: 85) | GCAGGAGGGGCCGGAGTATT (SEQ ID NO: 87) |
| GCAGGAGGGGCCGGAGTATT (SEQ ID NO: 87) | AGGGGCCGGAGTATTGGGAC (SEQ ID NO: 154) |
| AGGGGCCGGAGTATTGGGAC (SEQ ID NO: 154) | GGGGCCGGAGTATTGGGACC (SEQ ID NO: 89) |
| GGGGCCGGAGTATTGGGACC (SEQ ID NO: 89) | GTCTCCCGGTCCCAATACTC (SEQ ID NO: 298) |
| GTCTCCCGGTCCCAATACTC (SEQ ID NO: 298) | TCTTGGAGATCTGTGTCTCC (SEQ ID NO: 299) |
| TCTTGGAGATCTGTGTCTCC (SEQ ID NO: 299) | GTAAGTCTGTGTGTTGGTCT (SEQ ID NO: 300) |
| GTAAGTCTGTGTGTTGGTCT (SEQ ID NO: 300) | CTCTCGGTAAGTCTGTGTGT (SEQ ID NO: 301) |
| CTCTCGGTAAGTCTGTGTGT (SEQ ID NO: 301) | GCGCGGTGCGCAGGTTCTCT (SEQ ID NO: 302) |
| AGACTTACCGAGAGAGCCTG (SEQ ID NO: 320) | AGTAGCGGAGCGCGGTGCGC (SEQ ID NO: 303) |
| GCAGGTTCCGCAGGCTCTCT (SEQ ID NO: 252) | CTGGTTGTAGTAGCGGAGCG (SEQ ID NO: 304) |
| GAGAGCCTGCGGAACCTGCG (SEQ ID NO: 253) | CCGCTACTACAACCAGAGCG (SEQ ID NO: 152) |
| AGTAGCCGCGCAGGTTCCGC (SEQ ID NO: 254) | CCTCGCTCTGGTTGTAGTAG (SEQ ID NO: 153) |
| TCTGGTTGTAGTAGCCGCGC (SEQ ID NO: 114) | |
| CGGCTACTACAACCAGAGCG (SEQ ID NO: 115) | |

TABLE 3F
HLA-B 44:03 and 50:01

| 44:03 | 50:01 |
|---|---|
| AGCAGCAGGAGGAGGGTTCG (SEQ ID NO: 257) | AGCAGCAGGAGGACGGTTCG (SEQ ID NO: 209) |
| GAGCAGCAGGAGGAGGGTTC (SEQ ID NO: 258) | GAGCAGCAGGAGGACGGTTC (SEQ ID NO: 210) |
| AGAGCAGCAGGAGGAGGGTT (SEQ ID NO: 259) | AGAGCAGCAGGAGGACGGTT (SEQ ID NO: 211) |
| GAACCCTCCTCCTGCTGCTC (SEQ ID NO: 260) | AACCGTCCTCCTGCTGCTCT (SEQ ID NO: 212) |
| AACCCTCCTCCTGCTGCTCT (SEQ ID NO: 261) | CGCCGAGAGCAGCAGGAGGA (SEQ ID NO: 213) |
| ACCCTCCTCCTGCTGCTCTG (SEQ ID NO: 262) | CGTCCTCCTGCTGCTCTCGG (SEQ ID NO: 214) |
| CCCTCCTCCTGCTGCTCTGG (SEQ ID NO: 263) | GGGCCGCCGAGAGCAGCAGG (SEQ ID NO: 215) |
| CCCCCAGAGCAGCAGGAGGA (SEQ ID NO: 264) | CCTGCTGCTCTCGGCGGCCC (SEQ ID NO: 216) |
| CCTCCTCCTGCTGCTCTGGG (SEQ ID NO: 265) | CCAGGGCCGCCGAGAGCAGC (SEQ ID NO: 217) |
| CCCCCCAGAGCAGCAGGAGG (SEQ ID NO: 266) | CCCTGGCCCTGACCGAGACC (SEQ ID NO: 218) |
| CTGCCCCCCAGAGCAGCAGG (SEQ ID NO: 267) | CCAGGTCTCGGTCAGGGCCA (SEQ ID NO: 219) |
| CCTGCTGCTCTGGGGGGCAG (SEQ ID NO: 268) | CCTGGCCCTGACCGAGACCT (SEQ ID NO: 229) |
| CCACTGCCCCCCAGAGCAGC (SEQ ID NO: 269) | CCCAGGTCTCGGTCAGGGCC (SEQ ID NO: 230) |
| CAGTGGCCCTGACCGAGACC (SEQ ID NO: 270) | GTGGAAATACCTCATGGAGT (SEQ ID NO: 1012) |
| AGTGGCCCTGACCGAGACCT (SEQ ID NO: 271) | TGTGGAAATACCTCATGGAG (SEQ ID NO: 1013) |
| GTAGAAATACCTCATGGAGT (SEQ ID NO: 134) | GGCGGTGTGGAAATACCTCA (SEQ ID NO: 1014) |
| TGTAGAAATACCTCATGGAG (SEQ ID NO: 136) | ATTTCCACACCGCCATGTCC (SEQ ID NO: 1015) |
| GGCGGTGTAGAAATACCTCA (SEQ ID NO: 272) | CGGGCCGGGACATGGCGGTG (SEQ ID NO: 1016) |
| ATTTCTACACCGCCATGTCC (SEQ ID NO: 273) | CACACCGCCATGTCCCGGCC (SEQ ID NO: 1017) |
| TACACCGCCATGTCCCGGCC (SEQ ID NO: 274) | GCGGCCGGGCCGGGACATGG (SEQ ID NO: 225) |
| GCGGCCGGGCCGGGACATGG (SEQ ID NO: 225) | GCCATGTCCCGGCCCGGCCG (SEQ ID NO: 226) |
| GCCATGTCCCGGCCCGGCCG (SEQ ID NO: 226) | CCATGTCCCGGCCCGGCCGC (SEQ ID NO: 227) |
| CCATGTCCCGGCCCGGCCGC (SEQ ID NO: 227) | CCCGCGGCCGGGCCGGGACA (SEQ ID NO: 19) |
| CCCGCGGCCGGGCCGGGACA (SEQ ID NO: 19) | CATGTCCCGGCCCGGCCGCG (SEQ ID NO: 228) |
| CATGTCCCGGCCCGGCCGCG (SEQ ID NO: 228) | GGGCTCCCCGCGGCCGGGCC (SEQ ID NO: 25) |
| GGGCTCCCCGCGGCCGGGCC (SEQ ID NO: 25) | GGGGCTCCCCGCGGCCGGGC (SEQ ID NO: 28) |
| GGGGCTCCCCGCGGCCGGGC (SEQ ID NO: 28) | AAGCGGGGCTCCCCGCGGCC (SEQ ID NO: 31) |
| AAGCGGGGCTCCCCGCGGCC (SEQ ID NO: 31) | GAAGCGGGGCTCCCCGCGGC (SEQ ID NO: 34) |
| GAAGCGGGGCTCCCCGCGGC (SEQ ID NO: 34) | TGATGAAGCGGGGCTCCCCG (SEQ ID NO: 275) |
| TGATGAAGCGGGGCTCCCCG (SEQ ID NO: 275) | GGAGCCCCGCTTCATCACCG (SEQ ID NO: 276) |
| GGAGCCCCGCTTCATCACCG (SEQ ID NO: 276) | GAGCCCCGCTTCATCACCGT (SEQ ID NO: 277) |
| GAGCCCCGCTTCATCACCGT (SEQ ID NO: 277) | TAGCCCACGGTGATGAAGCG (SEQ ID NO: 278) |
| TAGCCCACGGTGATGAAGCG (SEQ ID NO: 278) | GTAGCCCACGGTGATGAAGC (SEQ ID NO: 279) |
| GTAGCCCACGGTGATGAAGC (SEQ ID NO: 279) | CGTAGCCCACGGTGATGAAG (SEQ ID NO: 280) |
| CGTAGCCCACGGTGATGAAG (SEQ ID NO: 280) | CTTCATCACCGTGGGCTACG (SEQ ID NO: 281) |
| CTTCATCACCGTGGGCTACG (SEQ ID NO: 281) | CGTGTCGTCCACGTAGCCCA (SEQ ID NO: 60) |
| CGTGTCGTCCACGTAGCCCA (SEQ ID NO: 60) | TGGACGACACGCTGTTCGTG (SEQ ID NO: 1018) |
| TGGACGACACGCTGTTCGTG (SEQ ID NO: 1018) | ACAGCGACGCCACGAGTCCG (SEQ ID NO: 283) |
| ACAGCGACGCCACGAGTCCG (SEQ ID NO: 283) | CGACGCCACGAGTCCGAGGA (SEQ ID NO: 284) |
| CGACGCCACGAGTCCGAGGA (SEQ ID NO: 284) | CGGCTCCTTCCTCGGACTCG (SEQ ID NO: 1019) |
| CGGCTCCTTCCTCGGACTCG (SEQ ID NO: 1019) | CGAGTCCGAGGAAGGAGCCG (SEQ ID NO: 1020) |
| CGAGTCCGAGGAAGGAGCCG (SEQ ID NO: 1020) | GAGTCCGAGGAAGGAGCCGC (SEQ ID NO: 1021) |
| GAGTCCGAGGAAGGAGCCGC (SEQ ID NO: 1021) | GGCGCCCGCGGCTCCTTCCT (SEQ ID NO: 1022) |
| GGCGCCCGCGGCTCCTTCCT (SEQ ID NO: 1022) | GGAAGGAGCCGCGGGCGCCA (SEQ ID NO: 1023) |
| GGAAGGAGCCGCGGGCGCCA (SEQ ID NO: 1023) | TGCTCTATCCATGGCGCCCG (SEQ ID NO: 290) |
| TGCTCTATCCATGGCGCCCG (SEQ ID NO: 290) | GCGGGCGCCATGGATAGAGC (SEQ ID NO: 1024) |
| GCGGGCGCCATGGATAGAGC (SEQ ID NO: 1024) | GGCGCCATGGATAGAGCAGG (SEQ ID NO: 294) |
| GGCGCCATGGATAGAGCAGG (SEQ ID NO: 294) | GCGCCATGGATAGAGCAGGA (SEQ ID NO: 295) |
| GCGCCATGGATAGAGCAGGA (SEQ ID NO: 295) | CGCCATGGATAGAGCAGGAG (SEQ ID NO: 296) |
| CGCCATGGATAGAGCAGGAG (SEQ ID NO: 296) | GGCCCCTCCTGCTCTATCCA (SEQ ID NO: 81) |
| GGCCCCTCCTGCTCTATCCA (SEQ ID NO: 81) | ATGGATAGAGCAGGAGGGGC (SEQ ID NO: 297) |
| ATGGATAGAGCAGGAGGGGC (SEQ ID NO: 297) | AGCAGGAGGGGCCGGAGTAT (SEQ ID NO: 85) |
| AGCAGGAGGGGCCGGAGTAT (SEQ ID NO: 85) | GCAGGAGGGGCCGGAGTATT (SEQ ID NO: 87) |
| GCAGGAGGGGCCGGAGTATT (SEQ ID NO: 87) | AGGGGCCGGAGTATTGGGAC (SEQ ID NO: 154) |
| AGGGGCCGGAGTATTGGGAC (SEQ ID NO: 154) | GGGGCCGGAGTATTGGGACC (SEQ ID NO: 89) |
| GGGGCCGGAGTATTGGGACC (SEQ ID NO: 89) | GTCTCCCGGTCCCAATACTC (SEQ ID NO: 298) |
| GTCTCCCGGTCCCAATACTC (SEQ ID NO: 298) | TCTTGGAGATCTGTGTCTCC (SEQ ID NO: 299) |
| TCTTGGAGATCTGTGTCTCC (SEQ ID NO: 299) | GTAAGTCTGTGTGTTGGTCT (SEQ ID NO: 300) |
| GTAAGTCTGTGTGTTGGTCT (SEQ ID NO: 300) | CTCTCGGTAAGTCTGTGTGT (SEQ ID NO: 301) |
| CTCTCGGTAAGTCTGTGTGT (SEQ ID NO: 301) | AGACTTACCGAGAGAGCCTG (SEQ ID NO: 320) |
| GCGCGGTGCGCAGGTTCTCT (SEQ ID NO: 302) | GCAGGTTCCGCAGGCTCTCT (SEQ ID NO: 252) |
| AGTAGCGGAGCGCGGTGCGC (SEQ ID NO: 303) | GAGAGCCTGCGGAACCTGCG (SEQ ID NO: 253) |
| CTGGTTGTAGTAGCGGAGCG (SEQ ID NO: 304) | AGTAGCCGCGCAGGTTCCGC (SEQ ID NO: 254) |
| CCGCTACTACAACCAGAGCG (SEQ ID NO: 152) | TCTGGTTGTAGTAGCCGCGC (SEQ ID NO: 114) |
| CCTCGCTCTGGTTGTAGTAG (SEQ ID NO: 153) | CGGCTACTACAACCAGAGCG (SEQ ID NO: 115) |

TABLE 3G
HLA-B 52:01 and 57:01

| 52:01 | 57:01 |
|---|---|
| AGCAGCAGGAGGACGGTTCG (SEQ ID NO: 209) | AGCAGCAGGAGGACGGTTCG (SEQ ID NO: 209) |
| GAGCAGCAGGAGGACGGTTC (SEQ ID NO: 210) | GAGCAGCAGGAGGACGGTTC (SEQ ID NO: 210) |
| AGAGCAGCAGGAGGACGGTT (SEQ ID NO: 211) | AGAGCAGCAGGAGGACGGTT (SEQ ID NO: 211) |
| GAACCGTCCTCCTGCTGCTC (SEQ ID NO: 967) | GAACCGTCCTCCTGCTGCTC (SEQ ID NO: 967) |
| AACCGTCCTCCTGCTGCTCT (SEQ ID NO: 212) | AACCGTCCTCCTGCTGCTCT (SEQ ID NO: 212) |
| ACCGTCCTCCTGCTGCTCTG (SEQ ID NO: 968) | ACCGTCCTCCTGCTGCTCTG (SEQ ID NO: 968) |
| CCGTCCTCCTGCTGCTCTGG (SEQ ID NO: 969) | CCGTCCTCCTGCTGCTCTGG (SEQ ID NO: 969) |
| CCCCCAGAGCAGCAGGAGGA (SEQ ID NO: 264) | CCCCCAGAGCAGCAGGAGGA (SEQ ID NO: 264) |
| CGTCCTCCTGCTGCTCTGGG (SEQ ID NO: 970) | CGTCCTCCTGCTGCTCTGGG (SEQ ID NO: 970) |
| CTGCCCCCCAGAGCAGCAGG (SEQ ID NO: 267) | CTGCCCCCCAGAGCAGCAGG (SEQ ID NO: 267) |
| CCTGCTGCTCTGGGGGGCAG (SEQ ID NO: 268) | CCTGCTGCTCTGGGGGGCAG (SEQ ID NO: 268) |
| CCACTGCCCCCCAGAGCAGC (SEQ ID NO: 269) | CCACTGCCCCCCAGAGCAGC (SEQ ID NO: 269) |
| CAGTGGCCCTGACCGAGACC (SEQ ID NO: 270) | CAGTGGCCCTGACCGAGACC (SEQ ID NO: 270) |
| AGTGGCCCTGACCGAGACCT (SEQ ID NO: 271) | AGTGGCCCTGACCGAGACCT (SEQ ID NO: 271) |
| GTAGAAATACCTCATGGAGT (SEQ ID NO: 134) | GTAGAAATACCTCATGGAGT (SEQ ID NO: 134) |
| TGTAGAAATACCTCATGGAG (SEQ ID NO: 136) | TGTAGAAATACCTCATGGAG (SEQ ID NO: 136) |
| GGCGGTGTAGAAATACCTCA (SEQ ID NO: 272) | GGCGGTGTAGAAATACCTCA (SEQ ID NO: 272) |
| ATTTCTACACCGCCATGTCC (SEQ ID NO: 273) | ATTTCTACACCGCCATGTCC (SEQ ID NO: 273) |
| TACACCGCCATGTCCCGGCC (SEQ ID NO: 274) | TACACCGCCATGTCCCGGCC (SEQ ID NO: 274) |
| GCGGCCGGGCCGGGACATGG (SEQ ID NO: 225) | GCGGCCGGGCCGGGACATGG (SEQ ID NO: 225) |
| GCCATGTCCCGGCCCGGCCG (SEQ ID NO: 226) | GCCATGTCCCGGCCCGGCCG (SEQ ID NO: 226) |
| CCATGTCCCGGCCCGGCCGC (SEQ ID NO: 227) | CCATGTCCCGGCCCGGCCGC (SEQ ID NO: 227) |
| CCCGCGGCCGGGCCGGGACA (SEQ ID NO: 19) | CCCGCGGCCGGGCCGGGACA (SEQ ID NO: 19) |
| CATGTCCCGGCCCGGCCGCG (SEQ ID NO: 228) | CATGTCCCGGCCCGGCCGCG (SEQ ID NO: 228) |
| GGGCTCCCCGCGGCCGGGCC (SEQ ID NO: 25) | GGGCTCCCCGCGGCCGGGCC (SEQ ID NO: 25) |
| GGGGCTCCCCGCGGCCGGGC (SEQ ID NO: 28) | GGGGCTCCCCGCGGCCGGGC (SEQ ID NO: 28) |
| AAGCGGGGCTCCCCGCGGCC (SEQ ID NO: 31) | AAGCGGGGCTCCCCGCGGCC (SEQ ID NO: 31) |
| GAAGCGGGGCTCCCCGCGGC (SEQ ID NO: 34) | GAAGCGGGGCTCCCCGCGGC (SEQ ID NO: 34) |
| CGATGAAGCGGGGCTCCCCG (SEQ ID NO: 37) | CGATGAAGCGGGGCTCCCCG (SEQ ID NO: 37) |
| GGAGCCCCGCTTCATCGCAG (SEQ ID NO: 40) | GGAGCCCCGCTTCATCGCAG (SEQ ID NO: 40) |
| GAGCCCCGCTTCATCGCAGT (SEQ ID NO: 43) | GAGCCCCGCTTCATCGCAGT (SEQ ID NO: 43) |
| TAGCCCACTGCGATGAAGCG (SEQ ID NO: 47) | TAGCCCACTGCGATGAAGCG (SEQ ID NO: 47) |
| GTAGCCCACTGCGATGAAGC (SEQ ID NO: 50) | GTAGCCCACTGCGATGAAGC (SEQ ID NO: 50) |
| CGTAGCCCACTGCGATGAAG (SEQ ID NO: 53) | CGTAGCCCACTGCGATGAAG (SEQ ID NO: 53) |
| CTTCATCGCAGTGGGCTACG (SEQ ID NO: 55) | CTTCATCGCAGTGGGCTACG (SEQ ID NO: 55) |
| TGGACGACACCCAGTTCGTG (SEQ ID NO: 238) | TGGACGACACCCAGTTCGTG (SEQ ID NO: 238) |
| GCTGTCGAACCTCACGAACT (SEQ ID NO: 239) | GCTGTCGAACCTCACGAACT (SEQ ID NO: 239) |
| CGCTGTCGAACCTCACGAAC (SEQ ID NO: 240) | CGCTGTCGAACCTCACGAAC (SEQ ID NO: 240) |
| ACAGCGACGCCGCGAGTCCG (SEQ ID NO: 318) | ACAGCGACGCCGCGAGTCCG (SEQ ID NO: 318) |
| CGACGCCGCGAGTCCGAGGA (SEQ ID NO: 319) | CGACGCCGCGAGTCCGAGGA (SEQ ID NO: 319) |
| GGGCTCCGTCCTCGGACTCG (SEQ ID NO: 323) | GGGCGCCATCCTCGGACTCG (SEQ ID NO: 285) |
| CGAGTCCGAGGACGGAGCCC (SEQ ID NO: 324) | CGAGTCCGAGGATGGCGCCC (SEQ ID NO: 286) |
| GAGTCCGAGGACGGAGCCCC (SEQ ID NO: 325) | GAGTCCGAGGATGGCGCCCC (SEQ ID NO: 287) |
| GGCGCCCGGGGCTCCGTCCT (SEQ ID NO: 326) | GGCGCCCGGGGCGCCATCCT (SEQ ID NO: 288) |
| GGACGGAGCCCCGGGCGCCA (SEQ ID NO: 1009) | GGATGGCGCCCCGGGCGCCA (SEQ ID NO: 289) |
| TGCTCTATCCATGGCGCCCG (SEQ ID NO: 290) | TGCTCTATCCATGGCGCCCG (SEQ ID NO: 290) |
| CTGCTCTATCCATGGCGCCC (SEQ ID NO: 291) | CTGCTCTATCCATGGCGCCC (SEQ ID NO: 291) |
| CCGGGCGCCATGGATAGAGC (SEQ ID NO: 292) | CCGGGCGCCATGGATAGAGC (SEQ ID NO: 292) |
| CCTGCTCTATCCATGGCGCC (SEQ ID NO: 293) | CCTGCTCTATCCATGGCGCC (SEQ ID NO: 293) |
| GGCGCCATGGATAGAGCAGG (SEQ ID NO: 294) | GGCGCCATGGATAGAGCAGG (SEQ ID NO: 294) |
| GCGCCATGGATAGAGCAGGA (SEQ ID NO: 295) | GCGCCATGGATAGAGCAGGA (SEQ ID NO: 295) |
| CGCCATGGATAGAGCAGGAG (SEQ ID NO: 296) | CGCCATGGATAGAGCAGGAG (SEQ ID NO: 296) |
| GGCCCCTCCTGCTCTATCCA (SEQ ID NO: 81) | GGCCCCTCCTGCTCTATCCA (SEQ ID NO: 81) |
| ATGGATAGAGCAGGAGGGGC (SEQ ID NO: 297) | ATGGATAGAGCAGGAGGGGC (SEQ ID NO: 297) |
| AGCAGGAGGGGCCGGAGTAT (SEQ ID NO: 85) | AGCAGGAGGGGCCGGAGTAT (SEQ ID NO: 85) |
| GCAGGAGGGGCCGGAGTATT (SEQ ID NO: 87) | GCAGGAGGGGCCGGAGTATT (SEQ ID NO: 87) |
| AGGGGCCGGAGTATTGGGAC (SEQ ID NO: 154) | GAGGGGCCGGAGTATTGGGA (SEQ ID NO: 1025) |
| GGGGCCGGAGTATTGGGACC (SEQ ID NO: 89) | AGGGGCCGGAGTATTGGGAC (SEQ ID NO: 154) |
| GTCTCCCGGTCCCAATACTC (SEQ ID NO: 298) | GGGGCCGGAGTATTGGGACG (SEQ ID NO: 142) |
| TCTTGGAGATCTGTGTCTCC (SEQ ID NO: 299) | GTCTCCCCGTCCCAATACTC (SEQ ID NO: 90) |
| GTAAGTCTGTGTGTTGGTCT (SEQ ID NO: 300) | AGTATTGGGACGGGGAGACA (SEQ ID NO: 92) |
| CTCTCGGTAAGTCTGTGTGT (SEQ ID NO: 301) | CGGGGAGACACGGAACATGA (SEQ ID NO: 1026) |
| AGACTTACCGAGAGAACCTG (SEQ ID NO: 1010) | CTCTCGGTAAGTCTGCGCGG (SEQ ID NO: 1027) |
| GCGCGATCCGCAGGTTCTCT (SEQ ID NO: 150) | GTTCTCTCGGTAAGTCTGCG (SEQ ID NO: 1028) |
| AGTAGCGGAGCGCGATCCGC (SEQ ID NO: 151) | AGACTTACCGAGAGAACCTG (SEQ ID NO: 1010) |
| CCGCTACTACAACCAGAGCG (SEQ ID NO: 152) | GCGCGATCCGCAGGTTCTCT (SEQ ID NO: 150) |
| CCTCGCTCTGGTTGTAGTAG (SEQ ID NO: 153) | AGTAGCGGAGCGCGATCCGC (SEQ ID NO: 151) |
| | CCGCTACTACAACCAGAGCG (SEQ ID NO: 152) |
| | CCTCGCTCTGGTTGTAGTAG (SEQ ID NO: 153) |

TABLE 4A
HLA-DQB1 02:01 and 03:01

| 02:01 | 03:01 |
|---|---|
| TGTCTTGGAAAAAGGCTTTG (SEQ ID NO: 1029) | TGTCTTGGAAAAAGGCTTTG (SEQ ID NO: 1029) |
| AAAAAGGCTTTGCGGATCCC (SEQ ID NO: 1030) | AAAAAGGCTTTGCGGATCCC (SEQ ID NO: 1030) |
| AAGGCTTTGCGGATCCCCGG (SEQ ID NO: 1031) | AAGGCTTTGCGGATCCCCGG (SEQ ID NO: 1031) |
| TGCGGATCCCCGGAGGCCTT (SEQ ID NO: 1032) | TGCGGATCCCCGGAGGCCTT (SEQ ID NO: 1032) |
| GCGGATCCCCGGAGGCCTTC (SEQ ID NO: 1033) | GCGGATCCCCGGAGGCCTTC (SEQ ID NO: 1033) |
| TTGCTGCCCGAAGGCCTCCG (SEQ ID NO: 1034) | TTGCTGCCCGAAGGCCTCCG (SEQ ID NO: 1034) |
| GTTGCTGCCCGAAGGCCTCC (SEQ ID NO: 1035) | GTTGCTGCCCGAAGGCCTCC (SEQ ID NO: 1035) |
| AGTTGCTGCCCGAAGGCCTC (SEQ ID NO: 1036) | AGTTGCTGCCCGAAGGCCTC (SEQ ID NO: 1036) |
| AGGTCACAGTTGCTGCCCGA (SEQ ID NO: 1037) | AGGTAACAGTTGCTGCCCGA (SEQ ID NO: 1083) |
| GCTCAGCATCGACAGCATCA (SEQ ID NO: 1038) | AGCAACTGTTACCTTGATGC (SEQ ID NO: 1084) |
| GTCGATGCTGAGCACCCCAG (SEQ ID NO: 1039) | GCTCAGCATCGCCAGCATCA (SEQ ID NO: 1085) |
| GCTGAGCACCCCAGTGGCTG (SEQ ID NO: 1040) | GGCGATGCTGAGCACCCCAG (SEQ ID NO: 1086) |
| CTGAGCACCCCAGTGGCTGA (SEQ ID NO: 1041) | GCTGAGCACCCCAGTGGCTG (SEQ ID NO: 1040) |
| GTCTCTGCCCTCAGCCACTG (SEQ ID NO: 1042) | CTGAGCACCCCAGTGGCTGA (SEQ ID NO: 1041) |
| AGTCTCTGCCCTCAGCCACT (SEQ ID NO: 1043) | GTCTCTGCCCTCAGCCACTG (SEQ ID NO: 1042) |
| GAGTCTCTGCCCTCAGCCAC (SEQ ID NO: 1044) | AGTCTCTGCCCTCAGCCACT (SEQ ID NO: 1043) |
| GGATTTCGTGTACCAGTTTA (SEQ ID NO: 1045) | GAGTCTCTGCCCTCAGCCAC (SEQ ID NO: 1044) |
| GATTTCGTGTACCAGTTTAA (SEQ ID NO: 1046) | GGATTTCGTGTACCAGTTTA (SEQ ID NO: 1045) |
| AGTAGCACATGCCCTTAAAC (SEQ ID NO: 1047) | AGTAGCACATGGCCTTAAAC (SEQ ID NO: 1087) |
| GGCATGTGCTACTTCACCAA (SEQ ID NO: 1048) | GCCATGTGCTACTTCACCAA (SEQ ID NO: 1088) |
| GCATGTGCTACTTCACCAAC (SEQ ID NO: 1049) | CCATGTGCTACTTCACCAAC (SEQ ID NO: 1089) |
| ACGCACGCGCTCTGTCCCGT (SEQ ID NO: 1050) | CCCGTTGGTGAAGTAGCACA (SEQ ID NO: 1090) |
| CGAAGCGCACGATCTCTTCT (SEQ ID NO: 1051) | GTGCTACTTCACCAACGGGA (SEQ ID NO: 1091) |
| CGTGCGCTTCGACAGCGACG (SEQ ID NO: 1052) | ACGCACGCGCTCCGTCCCGT (SEQ ID NO: 1092) |
| GTGCGCTTCGACAGCGACGT (SEQ ID NO: 1053) | CTCTCGGTTATAGATGTATC (SEQ ID NO: 1093) |
| TGCGCTTCGACAGCGACGTG (SEQ ID NO: 1054) | CAGATACATCTATAACCGAG (SEQ ID NO: 1094) |
| GCGCTTCGACAGCGACGTGG (SEQ ID NO: 1055) | CGAAGCGTGCGTACTCCTCT (SEQ ID NO: 1095) |
| ACAGCGACGTGGGGGAGTTC (SEQ ID NO: 1056) | CGCACGCTTCGACAGCGACG (SEQ ID NO: 1096) |
| CAGCGACGTGGGGGAGTTCC (SEQ ID NO: 1057) | ACGCTTCGACAGCGACGTGG (SEQ ID NO: 1097) |
| CGACGTGGGGGAGTTCCGGG (SEQ ID NO: 1058) | ACAGCGACGTGGAGGTGTAC (SEQ ID NO: 1098) |
| GTTCCGGGCGGTGACGCTGC (SEQ ID NO: 1059) | CAGCGACGTGGAGGTGTACC (SEQ ID NO: 1099) |
| TTCCGGGCGGTGACGCTGCT (SEQ ID NO: 1060) | CGACGTGGAGGTGTACCGGG (SEQ ID NO: 1100) |
| TCCGGGCGGTGACGCTGCTG (SEQ ID NO: 1061) | GTACCGGGCGGTGACGCCGC (SEQ ID NO: 1101) |
| GCCCCAGCAGCGTCACCGCC (SEQ ID NO: 1062) | TACCGGGCGGTGACGCCGCT (SEQ ID NO: 1102) |
| GGCTGCCTGCCGCCGAGTAC (SEQ ID NO: 1063) | ACCGGGCGGTGACGCCGCTG (SEQ ID NO: 1103) |
| CTGTTCCAGTACTCGGCGGC (SEQ ID NO: 1064) | GCCCCAGCGGCGTCACCGCC (SEQ ID NO: 1104) |
| CTGGCTGTTCCAGTACTCGG (SEQ ID NO: 1065) | TCGGCGTCAGGCGGCCCCAG (SEQ ID NO: 1105) |
| CTTCTGGCTGTTCCAGTACT (SEQ ID NO: 1066) | GGCCGCCTGACGCCGAGTAC (SEQ ID NO: 1106) |
| CGAGTACTGGAACAGCCAGA (SEQ ID NO: 1067) | TTCCAGTACTCGGCGTCAGG (SEQ ID NO: 1107) |
| GAACAGCCAGAAGGACATCC (SEQ ID NO: 1068) | CTGTTCCAGTACTCGGCGTC (SEQ ID NO: 1108) |
| GCCAGAAGGACATCCTGGAG (SEQ ID NO: 1069) | CTTCTGGCTGTTCCAGTACT (SEQ ID NO: 1066) |
| TCCTCTCCAGGATGTCCTTC (SEQ ID NO: 1070) | CGAGTACTGGAACAGCCAGA (SEQ ID NO: 1067) |
| AGGACATCCTGGAGAGGAAA (SEQ ID NO: 1071) | GAACAGCCAGAAGGAAGTCC (SEQ ID NO: 1109) |
| GGACATCCTGGAGAGGAAAC (SEQ ID NO: 1072) | GCCAGAAGGAAGTCCTGGAG (SEQ ID NO: 1110) |
| CATCCTGGAGAGGAAACGGG (SEQ ID NO: 1073) | TCCTCTCCAGGACTTCCTTC (SEQ ID NO: 1111) |
| CCTGGAGAGGAAACGGGCGG (SEQ ID NO: 1074) | AGGAAGTCCTGGAGAGGACC (SEQ ID NO: 1112) |
| CCGCCGCCCGTTTCCTCTCC (SEQ ID NO: 1075) | GGAAGTCCTGGAGAGGACCC (SEQ ID NO: 1113) |
| GGAGAGGAAACGGGCGGCGG (SEQ ID NO: 1076) | AGTCCTGGAGAGGACCCGGG (SEQ ID NO: 1114) |
| GGAAACGGGCGGCGGTGGAC (SEQ ID NO: 1077) | ACTCCGCCCGGGTCCTCTCC (SEQ ID NO: 1115) |
| GAAACGGGCGGCGGTGGACA (SEQ ID NO: 1078) | GGAGAGGACCCGGGCGGAGT (SEQ ID NO: 1116) |
| GTGCAGACACAACTACCAGT (SEQ ID NO: 1079) | GACCCGGGCGGAGTTGGACA (SEQ ID NO: 1117) |
| AGGTCGTGCGGAGCTCCAAC (SEQ ID NO: 1080) | CACCGTGTCCAACTCCGCCC (SEQ ID NO: 1118) |
| AGCTCCGCACGACCTTGCAG (SEQ ID NO: 1081) | ACACCGTGTCCAACTCCGCC (SEQ ID NO: 1119) |
| CTCGCCGCTGCAAGGTCGTG (SEQ ID NO: 1082) | GTGCAGACACAACTACCAGT (SEQ ID NO: 1079) |
| | AGGTCGTGCGGAGCTCCAAC (SEQ ID NO: 1080) |
| | AGCTCCGCACGACCTTGCAG (SEQ ID NO: 1081) |
| | CTCGCCGCTGCAAGGTCGTG (SEQ ID NO: 1082) |

TABLE 4B
HLA-DQB1 03:02 and 03:03

| 03:02 | 03:03 |
|---|---|
| TGTCTTGGAAGAAGGCTTTG (SEQ ID NO: 1120) | TGTCTTGGAAGAAGGCTTTG (SEQ ID NO: 1120) |
| AAGAAGGCTTTGCGGATCCC (SEQ ID NO: 1121) | AAGAAGGCTTTGCGGATCCC (SEQ ID NO: 1121) |
| AAGGCTTTGCGGATCCCTGG (SEQ ID NO: 1122) | AAGGCTTTGCGGATCCCTGG (SEQ ID NO: 1122) |
| TGCGGATCCCTGGAGGCCTT (SEQ ID NO: 1123) | TGCGGATCCCTGGAGGCCTT (SEQ ID NO: 1123) |
| GCGGATCCCTGGAGGCCTTC (SEQ ID NO: 1124) | GCGGATCCCTGGAGGCCTTC (SEQ ID NO: 1124) |
| TTGCTACCCGAAGGCCTCCA (SEQ ID NO: 1125) | TTGCTACCCGAAGGCCTCCA (SEQ ID NO: 1125) |
| GTTGCTACCCGAAGGCCTCC (SEQ ID NO: 1126) | GTTGCTACCCGAAGGCCTCC (SEQ ID NO: 1126) |
| AGGTCACAGTTGCTACCCGA (SEQ ID NO: 1127) | AGGTCACAGTTGCTACCCGA (SEQ ID NO: 1127) |
| AGCAACTGTGACCTTGATGC (SEQ ID NO: 1128) | AGCAACTGTGACCTTGATGC (SEQ ID NO: 1128) |
| GCTCAGCATCGCCAGCATCA (SEQ ID NO: 1085) | GCTCAGCATCGCCAGCATCA (SEQ ID NO: 1085) |
| GCTGGCGATGCTGAGCACCC (SEQ ID NO: 1129) | GCTGGCGATGCTGAGCACCC (SEQ ID NO: 1129) |
| GGCGATGCTGAGCACCCCGG (SEQ ID NO: 1130) | GGCGATGCTGAGCACCCCGG (SEQ ID NO: 1130) |
| GCTGAGCACCCCGGTGGCTG (SEQ ID NO: 1131) | GCTGAGCACCCCGGTGGCTG (SEQ ID NO: 1131) |
| CTGAGCACCCCGGTGGCTGA (SEQ ID NO: 1132) | CTGAGCACCCCGGTGGCTGA (SEQ ID NO: 1132) |
| GTCTCTGCCCTCAGCCACCG (SEQ ID NO: 1133) | GTCTCTGCCCTCAGCCACCG (SEQ ID NO: 1133) |
| AGTCTCTGCCCTCAGCCACC (SEQ ID NO: 1134) | AGTCTCTGCCCTCAGCCACC (SEQ ID NO: 1134) |
| GAGTCTCTGCCCTCAGCCAC (SEQ ID NO: 1044) | GAGTCTCTGCCCTCAGCCAC (SEQ ID NO: 1044) |
| GGATTTCGTGTACCAGTTTA (SEQ ID NO: 1045) | GGATTTCGTGTACCAGTTTA (SEQ ID NO: 1045) |
| GATTTCGTGTACCAGTTTAA (SEQ ID NO: 1046) | GATTTCGTGTACCAGTTTAA (SEQ ID NO: 1046) |
| AGTAGCACATGCCCTTAAAC (SEQ ID NO: 1047) | AGTAGCACATGCCCTTAAAC (SEQ ID NO: 1047) |
| GGCATGTGCTACTTCACCAA (SEQ ID NO: 1048) | GGCATGTGCTACTTCACCAA (SEQ ID NO: 1048) |
| GCATGTGCTACTTCACCAAC (SEQ ID NO: 1049) | GCATGTGCTACTTCACCAAC (SEQ ID NO: 1049) |
| GTGCTACTTCACCAACGGGA (SEQ ID NO: 1091) | GTGCTACTTCACCAACGGGA (SEQ ID NO: 1091) |
| ACGCACGCGCTCCGTCCCGT (SEQ ID NO: 1092) | ACGCACGCGCTCCGTCCCGT (SEQ ID NO: 1092) |
| CTCTCGGTTATAGATGTATC (SEQ ID NO: 1093) | CTCTCGGTTATAGATGTATC (SEQ ID NO: 1093) |
| CAGATACATCTATAACCGAG (SEQ ID NO: 1094) | CAGATACATCTATAACCGAG (SEQ ID NO: 1094) |
| CGAAGCGTGCGTACTCCTCT (SEQ ID NO: 1095) | CGAAGCGTGCGTACTCCTCT (SEQ ID NO: 1095) |
| CGCACGCTTCGACAGCGACG (SEQ ID NO: 1096) | CGCACGCTTCGACAGCGACG (SEQ ID NO: 1096) |
| GCACGCTTCGACAGCGACGT (SEQ ID NO: 1135) | GCACGCTTCGACAGCGACGT (SEQ ID NO: 1135) |
| CACGCTTCGACAGCGACGTG (SEQ ID NO: 1136) | CACGCTTCGACAGCGACGTG (SEQ ID NO: 1136) |
| ACGCTTCGACAGCGACGTGG (SEQ ID NO: 1097) | ACGCTTCGACAGCGACGTGG (SEQ ID NO: 1097) |
| ACAGCGACGTGGGGGTGTAT (SEQ ID NO: 1137) | ACAGCGACGTGGGGGTGTAT (SEQ ID NO: 1137) |
| CAGCGACGTGGGGGTGTATC (SEQ ID NO: 1138) | CAGCGACGTGGGGGTGTATC (SEQ ID NO: 1138) |
| CGACGTGGGGGTGTATCGGG (SEQ ID NO: 1139) | CGACGTGGGGGTGTATCGGG (SEQ ID NO: 1139) |
| GTATCGGGCGGTGACGCCGC (SEQ ID NO: 1140) | GTATCGGGCGGTGACGCCGC (SEQ ID NO: 1140) |
| TATCGGGCGGTGACGCCGCT (SEQ ID NO: 1141) | TATCGGGCGGTGACGCCGCT (SEQ ID NO: 1141) |
| ATCGGGCGGTGACGCCGCTG (SEQ ID NO: 1142) | ATCGGGCGGTGACGCCGCTG (SEQ ID NO: 1142) |
| TCGGCGGCAGGCGGCCCCAG (SEQ ID NO: 1143) | TCGGCGTCAGGCGGCCCCAG (SEQ ID NO: 1105) |
| GGCCGCCTGCCGCCGAGTAC (SEQ ID NO: 1144) | GGCCGCCTGACGCCGAGTAC (SEQ ID NO: 1106) |
| TTCCAGTACTCGGCGGCAGG (SEQ ID NO: 1145) | TTCCAGTACTCGGCGTCAGG (SEQ ID NO: 1107) |
| CTGTTCCAGTACTCGGCGGC (SEQ ID NO: 1064) | CTGTTCCAGTACTCGGCGTC (SEQ ID NO: 1108) |
| CTGGCTGTTCCAGTACTCGG (SEQ ID NO: 1065) | CTTCTGGCTGTTCCAGTACT (SEQ ID NO: 1066) |
| CTTCTGGCTGTTCCAGTACT (SEQ ID NO: 1066) | CGAGTACTGGAACAGCCAGA (SEQ ID NO: 1067) |
| CGAGTACTGGAACAGCCAGA (SEQ ID NO: 1067) | GAACAGCCAGAAGGAAGTCC (SEQ ID NO: 1109) |
| GAACAGCCAGAAGGAAGTCC (SEQ ID NO: 1109) | GCCAGAAGGAAGTCCTGGAG (SEQ ID NO: 1110) |
| GCCAGAAGGAAGTCCTGGAG (SEQ ID NO: 1110) | TCCTCTCCAGGACTTCCTTC (SEQ ID NO: 1111) |
| TCCTCTCCAGGACTTCCTTC (SEQ ID NO: 1111) | AGGAAGTCCTGGAGAGGACC (SEQ ID NO: 1112) |
| AGGAAGTCCTGGAGAGGACC (SEQ ID NO: 1112) | GGAAGTCCTGGAGAGGACCC (SEQ ID NO: 1113) |
| GGAAGTCCTGGAGAGGACCC (SEQ ID NO: 1113) | AGTCCTGGAGAGGACCCGGG (SEQ ID NO: 1114) |
| AGTCCTGGAGAGGACCCGGG (SEQ ID NO: 1114) | ACTCCGCCCGGGTCCTCTCC (SEQ ID NO: 1115) |
| ACTCCGCCCGGGTCCTCTCC (SEQ ID NO: 1115) | GGAGAGGACCCGGGCGGAGT (SEQ ID NO: 1116) |
| GGAGAGGACCCGGGCGGAGT (SEQ ID NO: 1116) | GACCCGGGCGGAGTTGGACA (SEQ ID NO: 1117) |
| GACCCGGGCGGAGTTGGACA (SEQ ID NO: 1117) | CACCGTGTCCAACTCCGCCC (SEQ ID NO: 1118) |
| CACCGTGTCCAACTCCGCCC (SEQ ID NO: 1118) | ACACCGTGTCCAACTCCGCC (SEQ ID NO: 1119) |
| ACACCGTGTCCAACTCCGCC (SEQ ID NO: 1119) | GTGCAGACACAACTACCAGT (SEQ ID NO: 1079) |
| GTGCAGACACAACTACCAGT (SEQ ID NO: 1079) | AGGTCGTGCGGAGCTCCAAC (SEQ ID NO: 1080) |
| AGGTCGTGCGGAGCTCCAAC (SEQ ID NO: 1080) | AGCTCCGCACGACCTTGCAG (SEQ ID NO: 1081) |
| AGCTCCGCACGACCTTGCAG (SEQ ID NO: 1081) | CTCGCCGCTGCAAGGTCGTG (SEQ ID NO: 1082) |
| CTCGCCGCTGCAAGGTCGTG (SEQ ID NO: 1082) | |

TABLE 4C
HLA-DQB1 05:01 and 05:03

| 05:01 | 05:03 |
|---|---|
| TGTCTTGGAAGAAGTCTTTG (SEQ ID NO: 1146) | TGTCTTGGAAGAAGTCTTTG (SEQ ID NO: 1146) |
| AAGAAGTCTTTGCGGATCCC (SEQ ID NO: 1147) | AAGAAGTCTTTGCGGATCCC (SEQ ID NO: 1147) |
| TGCGGATCCCCGGAGACCTT (SEQ ID NO: 1148) | TGCGGATCCCCGGAGACCTT (SEQ ID NO: 1148) |
| GCGGATCCCCGGAGACCTTC (SEQ ID NO: 1149) | GCGGATCCCCGGAGACCTTC (SEQ ID NO: 1149) |
| TTGCTACCCGAAGGTCTCCG (SEQ ID NO: 1150) | TTGCTACCCGAAGGTCTCCG (SEQ ID NO: 1150) |
| GTTGCTACCCGAAGGTCTCC (SEQ ID NO: 1151) | GTTGCTACCCGAAGGTCTCC (SEQ ID NO: 1151) |
| AGTTGCTACCCGAAGGTCTC (SEQ ID NO: 1152) | AGTTGCTACCCGAAGGTCTC (SEQ ID NO: 1152) |
| AGGTGACAGTTGCTACCCGA (SEQ ID NO: 1153) | AGGTGACAGTTGCTACCCGA (SEQ ID NO: 1153) |
| AGCAACTGTCACCTTGATGC (SEQ ID NO: 1154) | AGCAACTGTCACCTTGATGC (SEQ ID NO: 1154) |
| GCTCAGGATCGCCAGCATCA (SEQ ID NO: 1155) | GCTCAGGATCGCCAGCATCA (SEQ ID NO: 1155) |
| GGCGATCCTGAGCTCCTCAC (SEQ ID NO: 1156) | GGCGATCCTGAGCTCCTCAC (SEQ ID NO: 1156) |
| CCTGAGCTCCTCACTGGCTG (SEQ ID NO: 1157) | CCTGAGCTCCTCACTGGCTG (SEQ ID NO: 1157) |
| CCTCAGCCAGTGAGGAGCTC (SEQ ID NO: 1158) | CCTCAGCCAGTGAGGAGCTC (SEQ ID NO: 1158) |
| CTGAGCTCCTCACTGGCTGA (SEQ ID NO: 1159) | CTGAGCTCCTCACTGGCTGA (SEQ ID NO: 1159) |
| GTCTCTGCCCTCAGCCAGTG (SEQ ID NO: 1160) | GTCTCTGCCCTCAGCCAGTG (SEQ ID NO: 1160) |
| GGATTTCGTGTACCAGTTTA (SEQ ID NO: 1045) | GGATTTCGTGTACCAGTTTA (SEQ ID NO: 1045) |
| GATTTCGTGTACCAGTTTAA (SEQ ID NO: 1046) | GATTTCGTGTACCAGTTTAA (SEQ ID NO: 1046) |
| AGTAGCACAGGCCCTTAAAC (SEQ ID NO: 1161) | AGTAGCACAGGCCCTTAAAC (SEQ ID NO: 1161) |
| GGCCTGTGCTACTTCACCAA (SEQ ID NO: 1162) | GGCCTGTGCTACTTCACCAA (SEQ ID NO: 1162) |
| GCCTGTGCTACTTCACCAAC (SEQ ID NO: 1163) | GCCTGTGCTACTTCACCAAC (SEQ ID NO: 1163) |
| TCCCGTTGGTGAAGTAGCAC (SEQ ID NO: 1164) | TCCCGTTGGTGAAGTAGCAC (SEQ ID NO: 1164) |
| GTGCTACTTCACCAACGGGA (SEQ ID NO: 1091) | GTGCTACTTCACCAACGGGA (SEQ ID NO: 1091) |
| CCAACGGGACGGAGCGCGTG (SEQ ID NO: 1165) | CCAACGGGACGGAGCGCGTG (SEQ ID NO: 1165) |
| CCGCACGCGCTCCGTCCCGT (SEQ ID NO: 1166) | CCGCACGCGCTCCGTCCCGT (SEQ ID NO: 1166) |
| CAACGGGACGGAGCGCGTGC (SEQ ID NO: 1167) | CAACGGGACGGAGCGCGTGC (SEQ ID NO: 1167) |
| AACGGGACGGAGCGCGTGCG (SEQ ID NO: 1168) | AACGGGACGGAGCGCGTGCG (SEQ ID NO: 1168) |
| CTCTCGGTTATAGATGTGTC (SEQ ID NO: 1169) | CTCTCGGTTATAGATGTGTC (SEQ ID NO: 1169) |
| CAGACACATCTATAACCGAG (SEQ ID NO: 1170) | CAGACACATCTATAACCGAG (SEQ ID NO: 1170) |
| CGAAGCGCACGTACTCCTCT (SEQ ID NO: 1171) | CGAAGCGCACGTACTCCTCT (SEQ ID NO: 1171) |
| CGTGCGCTTCGACAGCGACG (SEQ ID NO: 1052) | CGTGCGCTTCGACAGCGACG (SEQ ID NO: 1052) |
| GTGCGCTTCGACAGCGACGT (SEQ ID NO: 1053) | GTGCGCTTCGACAGCGACGT (SEQ ID NO: 1053) |
| TGCGCTTCGACAGCGACGTG (SEQ ID NO: 1054) | TGCGCTTCGACAGCGACGTG (SEQ ID NO: 1054) |
| GCGCTTCGACAGCGACGTGG (SEQ ID NO: 1055) | GCGCTTCGACAGCGACGTGG (SEQ ID NO: 1055) |
| ACAGCGACGTGGGGGTGTAC (SEQ ID NO: 1172) | ACAGCGACGTGGGGGTGTAT (SEQ ID NO: 1137) |
| CAGCGACGTGGGGGTGTACC (SEQ ID NO: 1173) | CAGCGACGTGGGGGTGTATC (SEQ ID NO: 1138) |
| GTACCGGGCAGTGACGCCGC (SEQ ID NO: 1174) | CGACGTGGGGGTGTATCGGG (SEQ ID NO: 1139) |
| TACCGGGCAGTGACGCCGCA (SEQ ID NO: 1175) | GTATCGGGCGGTGACGCCGC (SEQ ID NO: 1140) |
| ACCGGGCAGTGACGCCGCAG (SEQ ID NO: 1176) | TATCGGGCGGTGACGCCGCA (SEQ ID NO: 1201) |
| GCCCCTGCGGCGTCACTGCC (SEQ ID NO: 1177) | ATCGGGCGGTGACGCCGCAG (SEQ ID NO: 1202) |
| GGGCAGTGACGCCGCAGGGG (SEQ ID NO: 1178) | GGGCGGTGACGCCGCAGGGG (SEQ ID NO: 1203) |
| TCGGCAACAGGCCGCCCCTG (SEQ ID NO: 1179) | TCGGCGTCAGGCCGCCCCTG (SEQ ID NO: 1204) |
| GGCGGCCTGTTGCCGAGTAC (SEQ ID NO: 1180) | GGCGGCCTGACGCCGAGTAC (SEQ ID NO: 1205) |
| CTGTTCCAGTACTCGGCAAC (SEQ ID NO: 1181) | CTGTTCCAGTACTCGGCGTC (SEQ ID NO: 1108) |
| CTTCTGGCTGTTCCAGTACT (SEQ ID NO: 1066) | CTTCTGGCTGTTCCAGTACT (SEQ ID NO: 1066) |
| CGAGTACTGGAACAGCCAGA (SEQ ID NO: 1067) | CGAGTACTGGAACAGCCAGA (SEQ ID NO: 1067) |
| GAACAGCCAGAAGGAAGTCC (SEQ ID NO: 1109) | GAACAGCCAGAAGGAAGTCC (SEQ ID NO: 1109) |
| CAGCCAGAAGGAAGTCCTGG (SEQ ID NO: 1182) | CAGCCAGAAGGAAGTCCTGG (SEQ ID NO: 1182) |
| AGCCAGAAGGAAGTCCTGGA (SEQ ID NO: 1183) | AGCCAGAAGGAAGTCCTGGA (SEQ ID NO: 1183) |
| GCCAGAAGGAAGTCCTGGAG (SEQ ID NO: 1110) | GCCAGAAGGAAGTCCTGGAG (SEQ ID NO: 1110) |
| CCAGAAGGAAGTCCTGGAGG (SEQ ID NO: 1184) | CCAGAAGGAAGTCCTGGAGG (SEQ ID NO: 1184) |
| CCCCCTCCAGGACTTCCTTC (SEQ ID NO: 1185) | CCCCCTCCAGGACTTCCTTC (SEQ ID NO: 1185) |
| AGGAAGTCCTGGAGGGGGCC (SEQ ID NO: 1186) | AGGAAGTCCTGGAGGGGGCC (SEQ ID NO: 1186) |
| GGAAGTCCTGGAGGGGGCCC (SEQ ID NO: 1187) | GGAAGTCCTGGAGGGGGCCC (SEQ ID NO: 1187) |
| CCTGGAGGGGGCCCGGGCGT (SEQ ID NO: 1188) | CCTGGAGGGGGCCCGGGCGT (SEQ ID NO: 1188) |
| CCGACGCCCGGGCCCCCTCC (SEQ ID NO: 1189) | CCGACGCCCGGGCCCCCTCC (SEQ ID NO: 1189) |
| GGAGGGGGCCCGGGCGTCGG (SEQ ID NO: 1190) | GGAGGGGGCCCGGGCGTCGG (SEQ ID NO: 1190) |
| GGGCCCGGGCGTCGGTGGAC (SEQ ID NO: 1191) | CACTCTGTCCACCGACGCCC (SEQ ID NO: 1206) |
| GGCCCGGGCGTCGGTGGACA (SEQ ID NO: 1192) | ACACTCTGTCCACCGACGCC (SEQ ID NO: 1207) |
| CACCCTGTCCACCGACGCCC (SEQ ID NO: 1193) | AGTGTGCAGACACAACTACG (SEQ ID NO: 1208) |
| ACACCCTGTCCACCGACGCC (SEQ ID NO: 1194) | GTGCAGACACAACTACGAGG (SEQ ID NO: 1196) |
| GGTGTGCAGACACAACTACG (SEQ ID NO: 1195) | AACTACGAGGTGGCGTACCG (SEQ ID NO: 1197) |
| GTGCAGACACAACTACGAGG (SEQ ID NO: 1196) | ACTACGAGGTGGCGTACCGC (SEQ ID NO: 1198) |
| AACTACGAGGTGGCGTACCG (SEQ ID NO: 1197) | CGTACCGCGGGATCCTGCAG (SEQ ID NO: 1199) |
| ACTACGAGGTGGCGTACCGC (SEQ ID NO: 1198) | CTCTCCTCTGCAGGATCCCG (SEQ ID NO: 1200) |
| CGTACCGCGGGATCCTGCAG (SEQ ID NO: 1199) | |
| CTCTCCTCTGCAGGATCCCG (SEQ ID NO: 1200) | |

TABLE 4D
HLA-DQB1 06:01 and 06:03

| 06:01 | 06:03 |
|---|---|
| TGTCTTGGAAGAAGGCTTTG (SEQ ID NO: 1120) | TGTCTTGGAAGAAGGCTTTG (SEQ ID NO: 1120) |
| AAGAAGGCTTTGCGGATCCC (SEQ ID NO: 1121) | AAGAAGGCTTTGCGGATCCC (SEQ ID NO: 1121) |
| TGCGGATCCCCGGAGACCTT (SEQ ID NO: 1148) | TGCGGATCCCCGGAGACCTT (SEQ ID NO: 1148) |
| GCGGATCCCCGGAGACCTTC (SEQ ID NO: 1149) | GCGGATCCCCGGAGACCTTC (SEQ ID NO: 1149) |
| TTGCTACCCGAAGGTCTCCG (SEQ ID NO: 1150) | TTGCTACCCGAAGGTCTCCG (SEQ ID NO: 1150) |
| GTTGCTACCCGAAGGTCTCC (SEQ ID NO: 1151) | GTTGCTACCCGAAGGTCTCC (SEQ ID NO: 1151) |
| AGTTGCTACCCGAAGGTCTC (SEQ ID NO: 1152) | AGTTGCTACCCGAAGGTCTC (SEQ ID NO: 1152) |
| AGGTGACAGTTGCTACCCGA (SEQ ID NO: 1153) | AGGTGACAGTTGCTACCCGA (SEQ ID NO: 1153) |
| AGCAACTGTCACCTTGATGC (SEQ ID NO: 1154) | AGCAACTGTCACCTTGATGC (SEQ ID NO: 1154) |
| GCTCAGCATCGCCAGCATCA (SEQ ID NO: 1085) | GCTCAGCATCGCCAGCATCA (SEQ ID NO: 1085) |
| GGCGATGCTGAGCTCCCTAC (SEQ ID NO: 1209) | GGCGATGCTGAGCTCCCTAC (SEQ ID NO: 1209) |
| GCTGAGCTCCCTACTGGCTG (SEQ ID NO: 1210) | GCTGAGCTCCCTACTGGCTG (SEQ ID NO: 1210) |
| CTGAGCTCCCTACTGGCTGA (SEQ ID NO: 1211) | CTGAGCTCCCTACTGGCTGA (SEQ ID NO: 1211) |
| GTCTCTGCCCTCAGCCAGTA (SEQ ID NO: 1212) | GTCTCTGCCCTCAGCCAGTA (SEQ ID NO: 1212) |
| AGTCTCTGCCCTCAGCCAGT (SEQ ID NO: 1213) | AGTCTCTGCCCTCAGCCAGT (SEQ ID NO: 1213) |
| GGATTTCGTGTTCCAGTTTA (SEQ ID NO: 1214) | GGATTTCGTGTACCAGTTTA (SEQ ID NO: 1045) |
| GATTTCGTGTTCCAGTTTAA (SEQ ID NO: 1215) | GATTTCGTGTACCAGTTTAA (SEQ ID NO: 1046) |
| AGTAGCACATGCCCTTAAAC (SEQ ID NO: 1047) | AGTAGCACATGCCCTTAAAC (SEQ ID NO: 1047) |
| GGCATGTGCTACTTCACCAA (SEQ ID NO: 1048) | GGCATGTGCTACTTCACCAA (SEQ ID NO: 1048) |
| GCATGTGCTACTTCACCAAC (SEQ ID NO: 1049) | GCATGTGCTACTTCACCAAC (SEQ ID NO: 1049) |
| GTGCTACTTCACCAACGGGA (SEQ ID NO: 1091) | GTGCTACTTCACCAACGGGA (SEQ ID NO: 1091) |
| ACGCACGCGCTCCGTCCCGT (SEQ ID NO: 1092) | ACGCACGCGCTCCGTCCCGT (SEQ ID NO: 1092) |
| CTCTCGGTTATAGATGTATC (SEQ ID NO: 1093) | CTCTCGGTTATAGATGTGTC (SEQ ID NO: 1169) |
| CAGATACATCTATAACCGAG (SEQ ID NO: 1094) | CAGACACATCTATAACCGAG (SEQ ID NO: 1170) |
| CGAAGCGCGCGTACTCCTCT (SEQ ID NO: 1216) | CGAAGCGCGCGTACTCCTCT (SEQ ID NO: 1216) |
| CGCGCGCTTCGACAGCGACG (SEQ ID NO: 1217) | CGCGCGCTTCGACAGCGACG (SEQ ID NO: 1217) |
| GCGCGCTTCGACAGCGACGT (SEQ ID NO: 1218) | GCGCGCTTCGACAGCGACGT (SEQ ID NO: 1218) |
| CGCGCTTCGACAGCGACGTG (SEQ ID NO: 1219) | CGCGCTTCGACAGCGACGTG (SEQ ID NO: 1219) |
| GCGCTTCGACAGCGACGTGG (SEQ ID NO: 1055) | GCGCTTCGACAGCGACGTGG (SEQ ID NO: 1055) |
| CGACGTGGGGGTGTACCGCG (SEQ ID NO: 1220) | CGACGTGGGGGTGTACCGCG (SEQ ID NO: 1220) |
| GTACCGCGCGGTGACGCCGC (SEQ ID NO: 1221) | GTACCGCGCGGTGACGCCGC (SEQ ID NO: 1221) |
| TACCGCGCGGTGACGCCGCA (SEQ ID NO: 1222) | TACCGCGCGGTGACGCCGCA (SEQ ID NO: 1222) |
| ACCGCGCGGTGACGCCGCAG (SEQ ID NO: 1223) | ACCGCGCGGTGACGCCGCAG (SEQ ID NO: 1223) |
| GCCCCTGCGGCGTCACCGCG (SEQ ID NO: 1224) | GCCCCTGCGGCGTCACCGCG (SEQ ID NO: 1224) |
| GCGCGGTGACGCCGCAGGGG (SEQ ID NO: 1225) | GCGCGGTGACGCCGCAGGGG (SEQ ID NO: 1225) |
| TCGGCATCAGGCCGCCCCTG (SEQ ID NO: 1226) | TCGGCATCAGGCCGCCCCTG (SEQ ID NO: 1226) |
| GGCGGCCTGATGCCGAGTAC (SEQ ID NO: 1227) | GGCGGCCTGATGCCGAGTAC (SEQ ID NO: 1227) |
| CTGTTCCAGTACTCGGCATC (SEQ ID NO: 1228) | CTGTTCCAGTACTCGGCATC (SEQ ID NO: 1228) |
| CTTCTGGCTGTTCCAGTACT (SEQ ID NO: 1066) | CTTCTGGCTGTTCCAGTACT (SEQ ID NO: 1066) |
| CGAGTACTGGAACAGCCAGA (SEQ ID NO: 1067) | CGAGTACTGGAACAGCCAGA (SEQ ID NO: 1067) |
| GAACAGCCAGAAGGAAGTCC (SEQ ID NO: 1109) | GAACAGCCAGAAGGAAGTCC (SEQ ID NO: 1109) |
| CAGCCAGAAGGAAGTCCTGG (SEQ ID NO: 1182) | CAGCCAGAAGGAAGTCCTGG (SEQ ID NO: 1182) |
| AGCCAGAAGGAAGTCCTGGA (SEQ ID NO: 1183) | AGCCAGAAGGAAGTCCTGGA (SEQ ID NO: 1183) |
| GCCAGAAGGAAGTCCTGGAG (SEQ ID NO: 1110) | GCCAGAAGGAAGTCCTGGAG (SEQ ID NO: 1110) |
| TCCCCTCCAGGACTTCCTTC (SEQ ID NO: 1229) | TCCCCTCCAGGACTTCCTTC (SEQ ID NO: 1229) |
| AGGAAGTCCTGGAGGGGACC (SEQ ID NO: 1230) | AGGAAGTCCTGGAGGGGACC (SEQ ID NO: 1230) |
| GGAAGTCCTGGAGGGGACCC (SEQ ID NO: 1231) | GGAAGTCCTGGAGGGGACCC (SEQ ID NO: 1231) |
| AGTCCTGGAGGGGACCCGGG (SEQ ID NO: 1232) | AGTCCTGGAGGGGACCCGGG (SEQ ID NO: 1232) |
| ACTCCGCCCGGGTCCCCTCC (SEQ ID NO: 1233) | ACTCCGCCCGGGTCCCCTCC (SEQ ID NO: 1233) |
| GGAGGGGACCCGGGCGGAGT (SEQ ID NO: 1234) | GGAGGGGACCCGGGCGGAGT (SEQ ID NO: 1234) |
| GACCCGGGCGGAGTTGGACA (SEQ ID NO: 1117) | GACCCGGGCGGAGTTGGACA (SEQ ID NO: 1117) |
| CACCGTGTCCAACTCCGCCC (SEQ ID NO: 1118) | CACCGTGTCCAACTCCGCCC (SEQ ID NO: 1118) |
| ACACCGTGTCCAACTCCGCC (SEQ ID NO: 1119) | ACACCGTGTCCAACTCCGCC (SEQ ID NO: 1119) |
| GGTGTGCAGACACAACTACG (SEQ ID NO: 1195) | GGTGTGCAGACACAACTACG (SEQ ID NO: 1195) |
| GTGCAGACACAACTACGAGG (SEQ ID NO: 1196) | GTGCAGACACAACTACGAGG (SEQ ID NO: 1196) |
| AACTACGAGGTGGCGTTCCG (SEQ ID NO: 1235) | AACTACGAGGTGGCGTTCCG (SEQ ID NO: 1235) |
| ACTACGAGGTGGCGTTCCGC (SEQ ID NO: 1236) | ACTACGAGGTGGCGTTCCGC (SEQ ID NO: 1236) |
| CGTTCCGCGGGATCTTGCAG (SEQ ID NO: 1237) | CGTTCCGCGGGATCTTGCAG (SEQ ID NO: 1237) |
| CTCTCCTCTGCAAGATCCCG (SEQ ID NO: 1238) | CTCTCCTCTGCAAGATCCCG (SEQ ID NO: 1238) |

TABLE 4E
HLA-DQB1 06:04
06:04

ATGGTGTGTCTGAAGCTCCC (SEQ ID NO: 1239)
GTGTGTCTGAAGCTCCCTGG (SEQ ID NO: 1240)
CTGTCATGCAGGAGCCTCCA (SEQ ID NO: 1241)
GCTGTCATGCAGGAGCCTCC (SEQ ID NO: 1242)
CACTGTCAGCGCTGTCATGC (SEQ ID NO: 1243)
AGCGCTGACAGTGACACTGA (SEQ ID NO: 1244)
GATGGTGCTGAGCTCCCCAC (SEQ ID NO: 1245)
GCTGAGCTCCCCACTGGCTT (SEQ ID NO: 1246)
AGCTCCCCACTGGCTTTGGC (SEQ ID NO: 1247)
GCTCCCCACTGGCTTTGGCT (SEQ ID NO: 1248)
CTCCCCACTGGCTTTGGCTG (SEQ ID NO: 1249)
GTCCCCAGCCAAAGCCAGTG (SEQ ID NO: 1250)
TGTCCCCAGCCAAAGCCAGT (SEQ ID NO: 1251)
GTGTCCCCAGCCAAAGCCAG (SEQ ID NO: 1252)
TTTGAATGTCATTTCTTCAA (SEQ ID NO: 1253)
TTGAATGTCATTTCTTCAAT (SEQ ID NO: 1254)
ATGTCATTTCTTCAATGGGA (SEQ ID NO: 1255)
ATTTCTTCAATGGGACGGAG (SEQ ID NO: 1256)
TTTCTTCAATGGGACGGAGC (SEQ ID NO: 1257)
TCAATGGGACGGAGCGGGTG (SEQ ID NO: 1258)
GACGGAGCGGGTGCGGTTGC (SEQ ID NO: 1259)
AAGATGCATCTATAACCAAG (SEQ ID NO: 1260)
CGAAGCGCACGGACTCCTCT (SEQ ID NO: 1261)
CACGTCGCTGTCGAAGCGCA (SEQ ID NO: 1262)
CGTGCGCTTCGACAGCGACG (SEQ ID NO: 1052)
GTGCGCTTCGACAGCGACGT (SEQ ID NO: 1053)
TGCGCTTCGACAGCGACGTG (SEQ ID NO: 1054)
GCGCTTCGACAGCGACGTGG (SEQ ID NO: 1055)
ACAGCGACGTGGGGGAGTAC (SEQ ID NO: 1263)
CAGCGACGTGGGGGAGTACC (SEQ ID NO: 1264)
CGACGTGGGGGAGTACCGGG (SEQ ID NO: 1265)
GGGGGAGTACCGGGCGGTGA (SEQ ID NO: 1266)
GTACCGGGCGGTGACGGAGC (SEQ ID NO: 1267)
TACCGGGCGGTGACGGAGCT (SEQ ID NO: 1268)
ACCGGGCGGTGACGGAGCTG (SEQ ID NO: 1269)
GCCCCAGCTCCGTCACCGCC (SEQ ID NO: 1270)
GGGCGGTGACGGAGCTGGGG (SEQ ID NO: 1271)
GGCGGCCTGATGCCGAGTAC (SEQ ID NO: 1227)
CTGTTCCAGTACTCGGCATC (SEQ ID NO: 1228)
CTTCTGGCTGTTCCAGTACT (SEQ ID NO: 1066)
CGAGTACTGGAACAGCCAGA (SEQ ID NO: 1067)
GAACAGCCAGAAGGACCTCC (SEQ ID NO: 1272)
TCTGCTCCAGGAGGTCCTTC (SEQ ID NO: 1273)
AGAAGGACCTCCTGGAGCAG (SEQ ID NO: 1274)
AGGACCTCCTGGAGCAGAGG (SEQ ID NO: 1275)
GGACCTCCTGGAGCAGAGGC (SEQ ID NO: 1276)
CGGCCCGCCTCTGCTCCAGG (SEQ ID NO: 1277)
CCTGGAGCAGAGGCGGGCCG (SEQ ID NO: 1278)
CCGCGGCCCGCCTCTGCTCC (SEQ ID NO: 1279)
GGAGCAGAGGCGGGCCGCGG (SEQ ID NO: 1280)
TCTGCAGTAGGTGTCCACCG (SEQ ID NO: 1281)
ACCTACTGCAGACACAACTA (SEQ ID NO: 1282)
CCTACTGCAGACACAACTAC (SEQ ID NO: 1283)
CCCGTAGTTGTGTCTGCAGT (SEQ ID NO: 1284)
CTACTGCAGACACAACTACG (SEQ ID NO: 1285)
TGCAGACACAACTACGGGGT (SEQ ID NO: 1286)
GTGAGAGCTTCACAGTGCAG (SEQ ID NO: 1287)

TABLE 5A
HLA-DRB1 01:02, 03:01, 01:01

| 01:02 | 03:01 | 01:01 |
|---|---|---|
| ATGGTGTGTCTGAAGCTCCC | ATGGTGTGTCTGAGGCTCCC | ATGGTGTGTCTGAAGCTCCC |
| (SEQ ID NO: 1239) | (SEQ ID NO: 1295) | (SEQ ID NO: 1239) |
| GTGTGTCTGAAGCTCCCTGG | GTGTGTCTGAGGCTCCCTGG | GTGTGTCTGAAGCTCCCTGG |
| (SEQ ID NO: 1240) | (SEQ ID NO: 1296) | (SEQ ID NO: 1240) |
| CTGTCATGCAGGAGCCTCCA | GCTCCCTGGAGGCTCCTGCA | CTGTCATGCAGGAGCCTCCA |
| (SEQ ID NO: 1241) | (SEQ ID NO: 1297) | (SEQ ID NO: 1241) |
| GCTGTCATGCAGGAGCCTCC | CTGCCATGCAGGAGCCTCCA | GCTGTCATGCAGGAGCCTCC |
| (SEQ ID NO: 1242) | (SEQ ID NO: 1298) | (SEQ ID NO: 1242) |
| CACTGTCAGCGCTGTCATGC | ACTGCCATGCAGGAGCCTCC | CACTGTCAGCGCTGTCATGC |
| (SEQ ID NO: 1243) | (SEQ ID NO: 1299) | (SEQ ID NO: 1243) |
| AGCGCTGACAGTGACACTGA | CACTGTCAGAACTGCCATGC | AGCGCTGACAGTGACACTGA |
| (SEQ ID NO: 1244) | (SEQ ID NO: 1300) | (SEQ ID NO: 1244) |
| GATGGTGCTGAGCTCCCCAC | AGTTCTGACAGTGACACTGA | GATGGTGCTGAGCTCCCCAC |
| (SEQ ID NO: 1245) | (SEQ ID NO: 1301) | (SEQ ID NO: 1245) |
| GCTGAGCTCCCCACTGGCTT | GATGGTGCTGAGCTCCCCAC | GCTGAGCTCCCCACTGGCTT |
| (SEQ ID NO: 1246) | (SEQ ID NO: 1245) | (SEQ ID NO: 1246) |
| AGCTCCCCACTGGCTTTGGC | GCTGAGCTCCCCACTGGCTT | AGCTCCCCACTGGCTTTGGC |
| (SEQ ID NO: 1247) | (SEQ ID NO: 1246) | (SEQ ID NO: 1247) |
| GCTCCCCACTGGCTTTGGCT | AGCTCCCCACTGGCTTTGGC | GCTCCCCACTGGCTTTGGCT |
| (SEQ ID NO: 1248) | (SEQ ID NO: 1247) | (SEQ ID NO: 1248) |
| CTCCCCACTGGCTTTGGCTG | GCTCCCCACTGGCTTTGGCT | CTCCCCACTGGCTTTGGCTG |
| (SEQ ID NO: 1249) | (SEQ ID NO: 1248) | (SEQ ID NO: 1249) |
| GTCCCCAGCCAAAGCCAGTG | CTCCCCACTGGCTTTGGCTG | GTCCCCAGCCAAAGCCAGTG |
| (SEQ ID NO: 1250) | (SEQ ID NO: 1249) | (SEQ ID NO: 1250) |
| TGTCCCCAGCCAAAGCCAGT | GTCCCCAGCCAAAGCCAGTG | TGTCCCCAGCCAAAGCCAGT |
| (SEQ ID NO: 1251) | (SEQ ID NO: 1250) | (SEQ ID NO: 1251) |
| GTGTCCCCAGCCAAAGCCAG | TGTCCCCAGCCAAAGCCAGT | GTGTCCCCAGCCAAAGCCAG |
| (SEQ ID NO: 1252) | (SEQ ID NO: 1251) | (SEQ ID NO: 1252) |
| TTTGAATGTCATTTCTTCAA | GTGTCCCCAGCCAAAGCCAG | TTTGAATGTCATTTCTTCAA |
| (SEQ ID NO: 1253) | (SEQ ID NO: 1252) | (SEQ ID NO: 1253) |
| TTGAATGTCATTTCTTCAAT | TCTGAGTGTCATTTCTTCAA | TTGAATGTCATTTCTTCAAT |
| (SEQ ID NO: 1254) | (SEQ ID NO: 1302) | (SEQ ID NO: 1254) |
| ATGTCATTTCTTCAATGGGA | CTGAGTGTCATTTCTTCAAT | ATGTCATTTCTTCAATGGGA |
| (SEQ ID NO: 1255) | (SEQ ID NO: 1303) | (SEQ ID NO: 1255) |
| ATTTCTTCAATGGGACGGAG | GTGTCATTTCTTCAATGGGA | ATTTCTTCAATGGGACGGAG |
| (SEQ ID NO: 1256) | (SEQ ID NO: 1304) | (SEQ ID NO: 1256) |
| TTTCTTCAATGGGACGGAGC | ATTTCTTCAATGGGACGGAG | TTTCTTCAATGGGACGGAGC |
| (SEQ ID NO: 1257) | (SEQ ID NO: 1256) | (SEQ ID NO: 1257) |
| TCAATGGGACGGAGCGGGTG | TTTCTTCAATGGGACGGAGC | TCAATGGGACGGAGCGGGTG |
| (SEQ ID NO: 1258) | (SEQ ID NO: 1257) | (SEQ ID NO: 1258) |
| GACGGAGCGGGTGCGGTTGC | TCAATGGGACGGAGCGGGTG | GACGGAGCGGGTGCGGTTGC |
| (SEQ ID NO: 1259) | (SEQ ID NO: 1258) | (SEQ ID NO: 1259) |
| AAGATGCATCTATAACCAAG | GACGGAGCGGGTGCGGTACC | AAGATGCATCTATAACCAAG |
| (SEQ ID NO: 1260) | (SEQ ID NO: 1305) | (SEQ ID NO: 1260) |
| CGAAGCGCACGGACTCCTCT | GGTTATGGAAGTATCTGTCC | CGAAGCGCACGGACTCCTCT |
| (SEQ ID NO: 1261) | (SEQ ID NO: 1306) | (SEQ ID NO: 1261) |
| CACGTCGCTGTCGAAGCGCA | GGACAGATACTTCCATAACC | CACGTCGCTGTCGAAGCGCA |
| (SEQ ID NO: 1262) | (SEQ ID NO: 1307) | (SEQ ID NO: 1262) |
| CGTGCGCTTCGACAGCGACG | CAGATACTTCCATAACCAGG | CGTGCGCTTCGACAGCGACG |
| (SEQ ID NO: 1052) | (SEQ ID NO: 1308) | (SEQ ID NO: 1052) |
| GTGCGCTTCGACAGCGACGT | GCACGTTCTCCTCCTGGTTA | GTGCGCTTCGACAGCGACGT |
| (SEQ ID NO: 1053) | (SEQ ID NO: 1309) | (SEQ ID NO: 1053) |
| TGCGCTTCGACAGCGACGTG | CGAAGCGCACGTTCTCCTCC | TGCGCTTCGACAGCGACGTG |
| (SEQ ID NO: 1054) | (SEQ ID NO: 1310) | (SEQ ID NO: 1054) |
| GCGCTTCGACAGCGACGTGG | CGTGCGCTTCGACAGCGACG | GCGCTTCGACAGCGACGTGG |
| (SEQ ID NO: 1055) | (SEQ ID NO: 1052) | (SEQ ID NO: 1055) |
| ACAGCGACGTGGGGGAGTAC | GTGCGCTTCGACAGCGACGT | ACAGCGACGTGGGGGAGTAC |
| (SEQ ID NO: 1263) | (SEQ ID NO: 1053) | (SEQ ID NO: 1263) |
| CAGCGACGTGGGGGAGTACC | TGCGCTTCGACAGCGACGTG | CAGCGACGTGGGGGAGTACC |
| (SEQ ID NO: 1264) | (SEQ ID NO: 1054) | (SEQ ID NO: 1264) |
| CGACGTGGGGGAGTACCGGG | GCGCTTCGACAGCGACGTGG | CGACGTGGGGGAGTACCGGG |
| (SEQ ID NO: 1265) | (SEQ ID NO: 1055) | (SEQ ID NO: 1265) |
| GGGGGAGTACCGGGCGGTGA | ACAGCGACGTGGGGGAGTTC | GGGGGAGTACCGGGCGGTGA |
| (SEQ ID NO: 1266) | (SEQ ID NO: 1056) | (SEQ ID NO: 1266) |
| GTACCGGGCGGTGACGGAGC | CAGCGACGTGGGGGAGTTCC | GTACCGGGCGGTGACGGAGC |
| (SEQ ID NO: 1267) | (SEQ ID NO: 1057) | (SEQ ID NO: 1267) |
| TACCGGGCGGTGACGGAGCT | CGACGTGGGGGAGTTCCGGG | TACCGGGCGGTGACGGAGCT |
| (SEQ ID NO: 1268) | (SEQ ID NO: 1058) | (SEQ ID NO: 1268) |
| ACCGGGCGGTGACGGAGCTG | GGGGGAGTTCCGGGCGGTGA | ACCGGGCGGTGACGGAGCTG |
| (SEQ ID NO: 1269) | (SEQ ID NO: 1311) | (SEQ ID NO: 1269) |
| GCCCCAGCTCCGTCACCGCC | GTTCCGGGCGGTGACGGAGC | GCCCCAGCTCCGTCACCGCC |
| (SEQ ID NO: 1270) | (SEQ ID NO: 1312) | (SEQ ID NO: 1270) |
| GGGCGGTGACGGAGCTGGGG | TTCCGGGCGGTGACGGAGCT | GGGCGGTGACGGAGCTGGGG |
| (SEQ ID NO: 1271) | (SEQ ID NO: 1313) | (SEQ ID NO: 1271) |
| GGCGGCCTGATGCCGAGTAC | TCCGGGCGGTGACGGAGCTG | GGCGGCCTGATGCCGAGTAC |
| (SEQ ID NO: 1227) | (SEQ ID NO: 1314) | (SEQ ID NO: 1227) |
| CTGTTCCAGTACTCGGCATC | GCCCCAGCTCCGTCACCGCC | CTGTTCCAGTACTCGGCATC |
| (SEQ ID NO: 1228) | (SEQ ID NO: 1270) | (SEQ ID NO: 1228) |
| CTTCTGGCTGTTCCAGTACT | GGGCGGTGACGGAGCTGGGG | CTTCTGGCTGTTCCAGTACT |
| (SEQ ID NO: 1066) | (SEQ ID NO: 1271) | (SEQ ID NO: 1066) |
| CGAGTACTGGAACAGCCAGA | GGCGGCCTGATGCCGAGTAC | CGAGTACTGGAACAGCCAGA |
| (SEQ ID NO: 1067) | (SEQ ID NO: 1227) | (SEQ ID NO: 1067) |
| GAACAGCCAGAAGGACCTCC | CTGTTCCAGTACTCGGCATC | GAACAGCCAGAAGGACCTCC |
| (SEQ ID NO: 1272) | (SEQ ID NO: 1228) | (SEQ ID NO: 1272) |
| TCTGCTCCAGGAGGTCCTTC | CTTCTGGCTGTTCCAGTACT | TCTGCTCCAGGAGGTCCTTC |
| (SEQ ID NO: 1273) | (SEQ ID NO: 1066) | (SEQ ID NO: 1273) |
| AGAAGGACCTCCTGGAGCAG | CGAGTACTGGAACAGCCAGA | AGAAGGACCTCCTGGAGCAG |
| (SEQ ID NO: 1274) | (SEQ ID NO: 1067) | (SEQ ID NO: 1274) |
| AGGACCTCCTGGAGCAGAGG | GAACAGCCAGAAGGACCTCC | AGGACCTCCTGGAGCAGAGG |
| (SEQ ID NO: 1275) | (SEQ ID NO: 1272) | (SEQ ID NO: 1275) |
| GGACCTCCTGGAGCAGAGGC | TCTGCTCCAGGAGGTCCTTC | GGACCTCCTGGAGCAGAGGC |
| (SEQ ID NO: 1276) | (SEQ ID NO: 1273) | (SEQ ID NO: 1276) |
| CGGCCCGCCTCTGCTCCAGG | AGGACCTCCTGGAGCAGAAG | CGGCCCGCCTCTGCTCCAGG |
| (SEQ ID NO: 1277) | (SEQ ID NO: 1315) | (SEQ ID NO: 1277) |
| CCTGGAGCAGAGGCGGGCCG | GGACCTCCTGGAGCAGAAGC | CCTGGAGCAGAGGCGGGCCG |
| (SEQ ID NO: 1278) | (SEQ ID NO: 1316) | (SEQ ID NO: 1278) |
| CCGCGGCCCGCCTCTGCTCC | GACCTCCTGGAGCAGAAGCG | CCGCGGCCCGCCTCTGCTCC |
| (SEQ ID NO: 1279) | (SEQ ID NO: 1317) | (SEQ ID NO: 1279) |
| GGAGCAGAGGCGGGCCGCGG | GGCCCCGCTTCTGCTCCAGG | GGAGCAGAGGCGGGCCGCGG |
| (SEQ ID NO: 1280) | (SEQ ID NO: 1318) | (SEQ ID NO: 1280) |
| TCTGCAATAGGTGTCCACCG | TCCTGGAGCAGAAGCGGGGC | TCTGCAGTAGGTGTCCACCG |
| (SEQ ID NO: 1288) | (SEQ ID NO: 1319) | (SEQ ID NO: 1281) |
| ACCTATTGCAGACACAACTA | CCTGGAGCAGAAGCGGGGCC | ACCTACTGCAGACACAACTA |
| (SEQ ID NO: 1289) | (SEQ ID NO: 1320) | (SEQ ID NO: 1282) |
| CCTATTGCAGACACAACTAC | CCCGGCCCCGCTTCTGCTCC | CCTACTGCAGACACAACTAC |
| (SEQ ID NO: 1290) | (SEQ ID NO: 1321) | (SEQ ID NO: 1283) |
| CCCGTAGTTGTGTCTGCAAT | GGAGCAGAAGCGGGGCCGGG | CCCGTAGTTGTGTCTGCAGT |
| (SEQ ID NO: 1291) | (SEQ ID NO: 1322) | (SEQ ID NO: 1284) |
| CTATTGCAGACACAACTACG | GTCTGCAGTAGTTGTCCACC | CTACTGCAGACACAACTACG |
| (SEQ ID NO: 1292) | (SEQ ID NO: 1323) | (SEQ ID NO: 1285) |
| CAGACACAACTACGGGGCTG | AACTACTGCAGACACAACTA | TGCAGACACAACTACGGGGT |
| (SEQ ID NO: 1293) | (SEQ ID NO: 1324) | (SEQ ID NO: 1286) |
| TGGAGAGCTTCACAGTGCAG | ACTACTGCAGACACAACTAC | GTGAGAGCTTCACAGTGCAG |
| (SEQ ID NO: 1294) | (SEQ ID NO: 1325) | (SEQ ID NO: 1287) |
| | CTACTGCAGACACAACTACG | |
| | (SEQ ID NO: 1285) | |
| | CAGACACAACTACGGGGTTG | |
| | (SEQ ID NO: 1326) | |
| | TGGAGAGCTTCACAGTGCAG | |
| | (SEQ ID NO: 1294) | |

TABLE 5B
HLA-DRB1 04:01 and 04:02

| 04:01 | 04:02 |
|---|---|
| ATGGTGTGTCTGAAGTTCCC (SEQ ID NO: 1327) | ATGGTGTGTCTGAAGTTCCC (SEQ ID NO: 1327) |
| GTGTGTCTGAAGTTCCCTGG (SEQ ID NO: 1328) | GTGTGTCTGAAGTTCCCTGG (SEQ ID NO: 1328) |
| GTTCCCTGGAGGCTCCTGCA (SEQ ID NO: 1329) | GTTCCCTGGAGGCTCCTGCA (SEQ ID NO: 1329) |
| CTGCCATGCAGGAGCCTCCA (SEQ ID NO: 1298) | CTGCCATGCAGGAGCCTCCA (SEQ ID NO: 1298) |
| GCTGCCATGCAGGAGCCTCC (SEQ ID NO: 1330) | GCTGCCATGCAGGAGCCTCC (SEQ ID NO: 1330) |
| CACTGTCAGAGCTGCCATGC (SEQ ID NO: 1331) | CACTGTCAGAGCTGCCATGC (SEQ ID NO: 1331) |
| AGCTCTGACAGTGACACTGA (SEQ ID NO: 1332) | AGCTCTGACAGTGACACTGA (SEQ ID NO: 1332) |
| GATGGTGCTGAGCTCCCCAC (SEQ ID NO: 1245) | GATGGTGCTGAGCTCCCCAC (SEQ ID NO: 1245) |
| GCTGAGCTCCCCACTGGCTT (SEQ ID NO: 1246) | GCTGAGCTCCCCACTGGCTT (SEQ ID NO: 1246) |
| AGCTCCCCACTGGCTTTGGC (SEQ ID NO: 1247) | AGCTCCCCACTGGCTTTGGC (SEQ ID NO: 1247) |
| GCTCCCCACTGGCTTTGGCT (SEQ ID NO: 1248) | GCTCCCCACTGGCTTTGGCT (SEQ ID NO: 1248) |
| CTCCCCACTGGCTTTGGCTG (SEQ ID NO: 1249) | CTCCCCACTGGCTTTGGCTG (SEQ ID NO: 1249) |
| GTCCCCAGCCAAAGCCAGTG (SEQ ID NO: 1250) | GTCCCCAGCCAAAGCCAGTG (SEQ ID NO: 1250) |
| TGTCCCCAGCCAAAGCCAGT (SEQ ID NO: 1251) | TGTCCCCAGCCAAAGCCAGT (SEQ ID NO: 1251) |
| GTGTCCCCAGCCAAAGCCAG (SEQ ID NO: 1252) | GTGTCCCCAGCCAAAGCCAG (SEQ ID NO: 1252) |
| CATGAGTGTCATTTCTTCAA (SEQ ID NO: 1333) | CATGAGTGTCATTTCTTCAA (SEQ ID NO: 1333) |
| ATGAGTGTCATTTCTTCAAC (SEQ ID NO: 1334) | ATGAGTGTCATTTCTTCAAC (SEQ ID NO: 1334) |
| GTGTCATTTCTTCAACGGGA (SEQ ID NO: 1335) | GTGTCATTTCTTCAACGGGA (SEQ ID NO: 1335) |
| ATTTCTTCAACGGGACGGAG (SEQ ID NO: 1336) | ATTTCTTCAACGGGACGGAG (SEQ ID NO: 1336) |
| TTTCTTCAACGGGACGGAGC (SEQ ID NO: 1337) | TTTCTTCAACGGGACGGAGC (SEQ ID NO: 1337) |
| TCAACGGGACGGAGCGGGTG (SEQ ID NO: 1338) | TCAACGGGACGGAGCGGGTG (SEQ ID NO: 1338) |
| GACGGAGCGGGTGCGGTTCC (SEQ ID NO: 1339) | GACGGAGCGGGTGCGGTTCC (SEQ ID NO: 1339) |
| GGTGATAGAAGTATCTGTCC (SEQ ID NO: 1340) | GGTGATAGAAGTATCTGTCC (SEQ ID NO: 1340) |
| CAGATACTTCTATCACCAAG (SEQ ID NO: 1341) | CAGATACTTCTATCACCAAG (SEQ ID NO: 1341) |
| CGAAGCGCACGTACTCCTCT (SEQ ID NO: 1171) | CGAAGCGCACGTACTCCTCT (SEQ ID NO: 1171) |
| CGTGCGCTTCGACAGCGACG (SEQ ID NO: 1052) | CGTGCGCTTCGACAGCGACG (SEQ ID NO: 1052) |
| GTGCGCTTCGACAGCGACGT (SEQ ID NO: 1053) | GTGCGCTTCGACAGCGACGT (SEQ ID NO: 1053) |
| TGCGCTTCGACAGCGACGTG (SEQ ID NO: 1054) | TGCGCTTCGACAGCGACGTG (SEQ ID NO: 1054) |
| GCGCTTCGACAGCGACGTGG (SEQ ID NO: 1055) | GCGCTTCGACAGCGACGTGG (SEQ ID NO: 1055) |
| ACAGCGACGTGGGGGAGTAC (SEQ ID NO: 1263) | ACAGCGACGTGGGGGAGTAC (SEQ ID NO: 1263) |
| CAGCGACGTGGGGGAGTACC (SEQ ID NO: 1264) | CAGCGACGTGGGGGAGTACC (SEQ ID NO: 1264) |
| CGACGTGGGGGAGTACCGGG (SEQ ID NO: 1265) | CGACGTGGGGGAGTACCGGG (SEQ ID NO: 1265) |
| GGGGGAGTACCGGGCGGTGA (SEQ ID NO: 1266) | GGGGGAGTACCGGGCGGTGA (SEQ ID NO: 1266) |
| GTACCGGGCGGTGACGGAGC (SEQ ID NO: 1267) | GTACCGGGCGGTGACGGAGC (SEQ ID NO: 1267) |
| TACCGGGCGGTGACGGAGCT (SEQ ID NO: 1268) | TACCGGGCGGTGACGGAGCT (SEQ ID NO: 1268) |
| ACCGGGCGGTGACGGAGCTG (SEQ ID NO: 1269) | ACCGGGCGGTGACGGAGCTG (SEQ ID NO: 1269) |
| GCCCCAGCTCCGTCACCGCC (SEQ ID NO: 1270) | GCCCCAGCTCCGTCACCGCC (SEQ ID NO: 1270) |
| GGGCGGTGACGGAGCTGGGG (SEQ ID NO: 1271) | GGGCGGTGACGGAGCTGGGG (SEQ ID NO: 1271) |
| GGCGGCCTGATGCCGAGTAC (SEQ ID NO: 1227) | GGCGGCCTGATGCCGAGTAC (SEQ ID NO: 1227) |
| CTGTTCCAGTACTCGGCATC (SEQ ID NO: 1228) | CTGTTCCAGTACTCGGCATC (SEQ ID NO: 1228) |
| CTTCTGGCTGTTCCAGTACT (SEQ ID NO: 1066) | CTTCTGGCTGTTCCAGTACT (SEQ ID NO: 1066) |
| CGAGTACTGGAACAGCCAGA (SEQ ID NO: 1067) | CGAGTACTGGAACAGCCAGA (SEQ ID NO: 1067) |
| GAACAGCCAGAAGGACCTCC (SEQ ID NO: 1272) | GAACAGCCAGAAGGACATCC (SEQ ID NO: 1068) |
| TCTGCTCCAGGAGGTCCTTC (SEQ ID NO: 1273) | CGTCTTCCAGGATGTCCTTC (SEQ ID NO: 1346) |
| AGGACCTCCTGGAGCAGAAG (SEQ ID NO: 1315) | AGGACATCCTGGAAGACGAG (SEQ ID NO: 1347) |
| GGACCTCCTGGAGCAGAAGC (SEQ ID NO: 1316) | GGACATCCTGGAAGACGAGC (SEQ ID NO: 1348) |
| CGGCCCGCTTCTGCTCCAGG (SEQ ID NO: 1342) | CCTGGAAGACGAGCGGGCCG (SEQ ID NO: 1349) |
| CCTGGAGCAGAAGCGGGCCG (SEQ ID NO: 1343) | CCGCGGCCCGCTCGTCTTCC (SEQ ID NO: 1350) |
| CCGCGGCCCGCTTCTGCTCC (SEQ ID NO: 1344) | GGAAGACGAGCGGGCCGCGG (SEQ ID NO: 1351) |
| GGAGCAGAAGCGGGCCGCGG (SEQ ID NO: 1345) | TCTGCAGTAGGTGTCCACCG (SEQ ID NO: 1281) |
| TCTGCAGTAGGTGTCCACCG (SEQ ID NO: 1281) | ACCTACTGCAGACACAACTA (SEQ ID NO: 1282) |
| ACCTACTGCAGACACAACTA (SEQ ID NO: 1282) | CCTACTGCAGACACAACTAC (SEQ ID NO: 1283) |
| CCTACTGCAGACACAACTAC (SEQ ID NO: 1283) | CCCGTAGTTGTGTCTGCAGT (SEQ ID NO: 1284) |
| CCCGTAGTTGTGTCTGCAGT (SEQ ID NO: 1284) | CTACTGCAGACACAACTACG (SEQ ID NO: 1285) |
| CTACTGCAGACACAACTACG (SEQ ID NO: 1285) | CAGACACAACTACGGGGTTG (SEQ ID NO: 1326) |
| TGCAGACACAACTACGGGGT (SEQ ID NO: 1286) | TGGAGAGCTTCACAGTGCAG (SEQ ID NO: 1294) |
| GTGAGAGCTTCACAGTGCAG (SEQ ID NO: 1287) | |

TABLE 5C
HLA-DRB1 04:04 and 07:01

| 04:04 | 07:01 |
|---|---|
| ATGGTGTGTCTGAAGTTCCC (SEQ ID NO: 1327) | ATGGTGTGTCTGAAGCTCCC (SEQ ID NO: 1239) |
| GTGTGTCTGAAGTTCCCTGG (SEQ ID NO: 1328) | GTGTGTCTGAAGCTCCCTGG (SEQ ID NO: 1240) |
| GTTCCCTGGAGGCTCCTGCA (SEQ ID NO: 1329) | GCTCCCTGGAGGCTCCTGCA (SEQ ID NO: 1297) |
| CTGCCATGCAGGAGCCTCCA (SEQ ID NO: 1298) | CTGCCATGCAGGAGCCTCCA (SEQ ID NO: 1298) |
| GCTGCCATGCAGGAGCCTCC (SEQ ID NO: 1330) | GCTGCCATGCAGGAGCCTCC (SEQ ID NO: 1330) |
| CACTGTCAGAGCTGCCATGC (SEQ ID NO: 1331) | CACTGTCAGAGCTGCCATGC (SEQ ID NO: 1331) |
| AGCTCTGACAGTGACACTGA (SEQ ID NO: 1332) | AGCTCTGACAGTGACACTGA (SEQ ID NO: 1332) |
| GATGGTGCTGAGCTCCCCAC (SEQ ID NO: 1245) | GATGGTGCTGAGCTCCCCAC (SEQ ID NO: 1245) |
| GCTGAGCTCCCCACTGGCTT (SEQ ID NO: 1246) | GCTGAGCTCCCCACTGGCTT (SEQ ID NO: 1246) |
| AGCTCCCCACTGGCTTTGGC (SEQ ID NO: 1247) | AGCTCCCCACTGGCTTTGGC (SEQ ID NO: 1247) |
| GCTCCCCACTGGCTTTGGCT (SEQ ID NO: 1248) | GCTCCCCACTGGCTTTGGCT (SEQ ID NO: 1248) |
| CTCCCCACTGGCTTTGGCTG (SEQ ID NO: 1249) | CTCCCCACTGGCTTTGGCTG (SEQ ID NO: 1249) |
| GTCCCCAGCCAAAGCCAGTG (SEQ ID NO: 1250) | GTCCCCAGCCAAAGCCAGTG (SEQ ID NO: 1250) |
| TGTCCCCAGCCAAAGCCAGT (SEQ ID NO: 1251) | TGTCCCCAGCCAAAGCCAGT (SEQ ID NO: 1251) |
| GTGTCCCCAGCCAAAGCCAG (SEQ ID NO: 1252) | GTGTCCCCAGCCAAAGCCAG (SEQ ID NO: 1252) |
| CATGAGTGTCATTTCTTCAA (SEQ ID NO: 1333) | ACTTATACTTACCCTGCCAC (SEQ ID NO: 1352) |
| ATGAGTGTCATTTCTTCAAC (SEQ ID NO: 1334) | TATAAGTGTCATTTCTTCAA (SEQ ID NO: 1353) |
| GTGTCATTTCTTCAACGGGA (SEQ ID NO: 1335) | ATAAGTGTCATTTCTTCAAC (SEQ ID NO: 1354) |
| ATTTCTTCAACGGGACGGAG (SEQ ID NO: 1336) | GTGTCATTTCTTCAACGGGA (SEQ ID NO: 1335) |
| TTTCTTCAACGGGACGGAGC (SEQ ID NO: 1337) | ATTTCTTCAACGGGACGGAG (SEQ ID NO: 1336) |
| TCAACGGGACGGAGCGGGTG (SEQ ID NO: 1338) | TTTCTTCAACGGGACGGAGC (SEQ ID NO: 1337) |
| GACGGAGCGGGTGCGGTTCC (SEQ ID NO: 1339) | GACGGAGCGGGTGCAGTTCC (SEQ ID NO: 1355) |
| GGTGATAGAAGTATCTGTCC (SEQ ID NO: 1340) | GGTTATAGAAGAGTCTTTCC (SEQ ID NO: 1356) |
| CAGATACTTCTATCACCAAG (SEQ ID NO: 1341) | GGAAAGACTCTTCTATAACC (SEQ ID NO: 1357) |
| CGAAGCGCACGTACTCCTCT (SEQ ID NO: 1171) | AAGACTCTTCTATAACCAGG (SEQ ID NO: 1358) |
| CGTGCGCTTCGACAGCGACG (SEQ ID NO: 1052) | CGAAGCGCACGAACTCCTCC (SEQ ID NO: 1359) |
| GTGCGCTTCGACAGCGACGT (SEQ ID NO: 1053) | CGTGCGCTTCGACAGCGACG (SEQ ID NO: 1052) |
| TGCGCTTCGACAGCGACGTG (SEQ ID NO: 1054) | GTGCGCTTCGACAGCGACGT (SEQ ID NO: 1053) |
| GCGCTTCGACAGCGACGTGG (SEQ ID NO: 1055) | TGCGCTTCGACAGCGACGTG (SEQ ID NO: 1054) |
| ACAGCGACGTGGGGGAGTAC (SEQ ID NO: 1263) | GCGCTTCGACAGCGACGTGG (SEQ ID NO: 1055) |
| CAGCGACGTGGGGGAGTACC (SEQ ID NO: 1264) | ACAGCGACGTGGGGGAGTAC (SEQ ID NO: 1263) |
| CGACGTGGGGGAGTACCGGG (SEQ ID NO: 1265) | CAGCGACGTGGGGGAGTACC (SEQ ID NO: 1264) |
| GGGGGAGTACCGGGCGGTGA (SEQ ID NO: 1266) | CGACGTGGGGGAGTACCGGG (SEQ ID NO: 1265) |
| GTACCGGGCGGTGACGGAGC (SEQ ID NO: 1267) | GGGGGAGTACCGGGCGGTGA (SEQ ID NO: 1266) |
| TACCGGGCGGTGACGGAGCT (SEQ ID NO: 1268) | TACCGGGCGGTGACGGAGCT (SEQ ID NO: 1268) |
| ACCGGGCGGTGACGGAGCTG (SEQ ID NO: 1269) | ACCGGGCGGTGACGGAGCTA (SEQ ID NO: 1360) |
| GCCCCAGCTCCGTCACCGCC (SEQ ID NO: 1270) | GCCCTAGCTCCGTCACCGCC (SEQ ID NO: 1361) |
| GGGCGGTGACGGAGCTGGGG (SEQ ID NO: 1271) | GGGCGGTGACGGAGCTAGGG (SEQ ID NO: 1362) |
| GGCGGCCTGATGCCGAGTAC (SEQ ID NO: 1227) | GGCGGCCTGTCGCCGAGTCC (SEQ ID NO: 1363) |
| CTGTTCCAGTACTCGGCATC (SEQ ID NO: 1228) | CTGTTCCAGGACTCGGCGAC (SEQ ID NO: 1364) |
| CTTCTGGCTGTTCCAGTACT (SEQ ID NO: 1066) | CTTCTGGCTGTTCCAGGACT (SEQ ID NO: 1365) |
| CGAGTACTGGAACAGCCAGA (SEQ ID NO: 1067) | CGAGTCCTGGAACAGCCAGA (SEQ ID NO: 1366) |
| GAACAGCCAGAAGGACCTCC (SEQ ID NO: 1272) | GATGTCCTTCTGGCTGTTCC (SEQ ID NO: 1367) |
| TCTGCTCCAGGAGGTCCTTC (SEQ ID NO: 1273) | GAACAGCCAGAAGGACATCC (SEQ ID NO: 1068) |
| AGAAGGACCTCCTGGAGCAG (SEQ ID NO: 1274) | CAGCCAGAAGGACATCCTGG (SEQ ID NO: 1368) |
| AGGACCTCCTGGAGCAGAGG (SEQ ID NO: 1275) | TGTCCTCCAGGATGTCCTTC (SEQ ID NO: 1369) |
| GGACCTCCTGGAGCAGAGGC (SEQ ID NO: 1276) | AGAAGGACATCCTGGAGGAC (SEQ ID NO: 1370) |
| CGGCCCGCCTCTGCTCCAGG (SEQ ID NO: 1277) | AGGACATCCTGGAGGACAGG (SEQ ID NO: 1371) |
| CCTGGAGCAGAGGCGGGCCG (SEQ ID NO: 1278) | GGACATCCTGGAGGACAGGC (SEQ ID NO: 1373) |
| CCGCGGCCCGCCTCTGCTCC (SEQ ID NO: 1279) | GACATCCTGGAGGACAGGCG (SEQ ID NO: 1374) |
| GGAGCAGAGGCGGGCCGCGG (SEQ ID NO: 1280) | CCTGGAGGACAGGCGGGGCC (SEQ ID NO: 1375) |
| TCTGCAGTAGGTGTCCACCG (SEQ ID NO: 1281) | CCTGGCCCCGCCTGTCCTCC (SEQ ID NO: 1376) |
| ACCTACTGCAGACACAACTA (SEQ ID NO: 1282) | GGAGGACAGGCGGGGCCAGG (SEQ ID NO: 1377) |
| CCTACTGCAGACACAACTAC (SEQ ID NO: 1283) | GTCTGCACACGGTGTCCACC (SEQ ID NO: 1378) |
| CCCGTAGTTGTGTCTGCAGT (SEQ ID NO: 1284) | ACCGTGTGCAGACACAACTA (SEQ ID NO: 1379) |
| CTACTGCAGACACAACTACG (SEQ ID NO: 1285) | CCGTGTGCAGACACAACTAC (SEQ ID NO: 1380) |
| CAGACACAACTACGGGGTTG (SEQ ID NO: 1326) | CCCGTAGTTGTGTCTGCACA (SEQ ID NO: 1381) |
| TGGAGAGCTTCACAGTGCAG (SEQ ID NO: 1294) | CGTGTGCAGACACAACTACG (SEQ ID NO: 1382) |
| | TGCAGACACAACTACGGGGT (SEQ ID NO: 1286) |
| | GTGAGAGCTTCACAGTGCAG (SEQ ID NO: 1287) |

TABLE 5D
HLA-DRB1 11:04 and 13:01

| 11:04 | 13:01 |
|---|---|
| ATGGTGTGTCTGAGGCTCCC (SEQ ID NO: 1295) | ATGGTGTGTCTGAGGCTCCC (SEQ ID NO: 1295) |
| GTGTGTCTGAGGCTCCCTGG (SEQ ID NO: 1296) | GTGTGTCTGAGGCTCCCTGG (SEQ ID NO: 1296) |
| GCTCCCTGGAGGCTCCTGCA (SEQ ID NO: 1297) | GCTCCCTGGAGGCTCCTGCA (SEQ ID NO: 1297) |
| CTGCCATGCAGGAGCCTCCA (SEQ ID NO: 1298) | CTGCCATGCAGGAGCCTCCA (SEQ ID NO: 1298) |
| ACTGCCATGCAGGAGCCTCC (SEQ ID NO: 1299) | ACTGCCATGCAGGAGCCTCC (SEQ ID NO: 1299) |
| CACTGTCAGAACTGCCATGC (SEQ ID NO: 1300) | CACTGTCAGAACTGCCATGC (SEQ ID NO: 1300) |
| AGTTCTGACAGTGACACTGA (SEQ ID NO: 1301) | AGTTCTGACAGTGACACTGA (SEQ ID NO: 1301) |
| GATGGTGCTGAGCTCCCCAC (SEQ ID NO: 1245) | GATGGTGCTGAGCTCCCCAC (SEQ ID NO: 1245) |
| GCTGAGCTCCCCACTGGCTT (SEQ ID NO: 1246) | GCTGAGCTCCCCACTGGCTT (SEQ ID NO: 1246) |
| AGCTCCCCACTGGCTTTGGC (SEQ ID NO: 1247) | AGCTCCCCACTGGCTTTGGC (SEQ ID NO: 1247) |
| GCTCCCCACTGGCTTTGGCT (SEQ ID NO: 1248) | GCTCCCCACTGGCTTTGGCT (SEQ ID NO: 1248) |
| CTCCCCACTGGCTTTGGCTG (SEQ ID NO: 1249) | CTCCCCACTGGCTTTGGCTG (SEQ ID NO: 1249) |
| GTCCCCAGCCAAAGCCAGTG (SEQ ID NO: 1250) | GTCCCCAGCCAAAGCCAGTG (SEQ ID NO: 1250) |
| TGTCCCCAGCCAAAGCCAGT (SEQ ID NO: 1251) | TGTCCCCAGCCAAAGCCAGT (SEQ ID NO: 1251) |
| GTGTCCCCAGCCAAAGCCAG (SEQ ID NO: 1252) | GTGTCCCCAGCCAAAGCCAG (SEQ ID NO: 1252) |
| TCTGAGTGTCATTTCTTCAA (SEQ ID NO: 1302) | TCTGAGTGTCATTTCTTCAA (SEQ ID NO: 1302) |
| CTGAGTGTCATTTCTTCAAT (SEQ ID NO: 1303) | CTGAGTGTCATTTCTTCAAT (SEQ ID NO: 1303) |
| GTGTCATTTCTTCAATGGGA (SEQ ID NO: 1304) | GTGTCATTTCTTCAATGGGA (SEQ ID NO: 1304) |
| ATTTCTTCAATGGGACGGAG (SEQ ID NO: 1256) | ATTTCTTCAATGGGACGGAG (SEQ ID NO: 1256) |
| TTTCTTCAATGGGACGGAGC (SEQ ID NO: 1257) | TTTCTTCAATGGGACGGAGC (SEQ ID NO: 1257) |
| TCAATGGGACGGAGCGGGTG (SEQ ID NO: 1258) | TCAATGGGACGGAGCGGGTG (SEQ ID NO: 1258) |
| GACGGAGCGGGTGCGGTTCC (SEQ ID NO: 1339) | GACGGAGCGGGTGCGGTTCC (SEQ ID NO: 1339) |
| GGTTATAGAAGTATCTGTCC (SEQ ID NO: 1383) | GGTTATGGAAGTATCTGTCC (SEQ ID NO: 1306) |
| CAGATACTTCTATAACCAAG (SEQ ID NO: 1384) | GGACAGATACTTCCATAACC (SEQ ID NO: 1307) |
| CGAAGCGCACGTACTCCTCT (SEQ ID NO: 1171) | CAGATACTTCCATAACCAGG (SEQ ID NO: 1308) |
| CGTGCGCTTCGACAGCGACG (SEQ ID NO: 1052) | GCACGTTCTCCTCCTGGTTA (SEQ ID NO: 1309) |
| GTGCGCTTCGACAGCGACGT (SEQ ID NO: 1053) | CGAAGCGCACGTTCTCCTCC (SEQ ID NO: 1310) |
| TGCGCTTCGACAGCGACGTG (SEQ ID NO: 1054) | CGTGCGCTTCGACAGCGACG (SEQ ID NO: 1052) |
| GCGCTTCGACAGCGACGTGG (SEQ ID NO: 1055) | GTGCGCTTCGACAGCGACGT (SEQ ID NO: 1053) |
| ACAGCGACGTGGGGGAGTTC (SEQ ID NO: 1056) | TGCGCTTCGACAGCGACGTG (SEQ ID NO: 1054) |
| CAGCGACGTGGGGGAGTTCC (SEQ ID NO: 1057) | GCGCTTCGACAGCGACGTGG (SEQ ID NO: 1055) |
| CGACGTGGGGGAGTTCCGGG (SEQ ID NO: 1058) | ACAGCGACGTGGGGGAGTTC (SEQ ID NO: 1056) |
| GGGGGAGTTCCGGGCGGTGA (SEQ ID NO: 1311) | CAGCGACGTGGGGGAGTTCC (SEQ ID NO: 1057) |
| GTTCCGGGCGGTGACGGAGC (SEQ ID NO: 1312) | CGACGTGGGGGAGTTCCGGG (SEQ ID NO: 1058) |
| TTCCGGGCGGTGACGGAGCT (SEQ ID NO: 1313) | GGGGGAGTTCCGGGCGGTGA (SEQ ID NO: 1311) |
| TCCGGGCGGTGACGGAGCTG (SEQ ID NO: 1314) | GTTCCGGGCGGTGACGGAGC (SEQ ID NO: 1312) |
| GCCCCAGCTCCGTCACCGCC (SEQ ID NO: 1270) | TTCCGGGCGGTGACGGAGCT (SEQ ID NO: 1313) |
| GGGCGGTGACGGAGCTGGGG (SEQ ID NO: 1271) | TCCGGGCGGTGACGGAGCTG (SEQ ID NO: 1314) |
| GGAGCTGGGGCGGCCTGATG (SEQ ID NO: 1385) | GCCCCAGCTCCGTCACCGCC (SEQ ID NO: 1270) |
| GGCGGCCTGATGAGGAGTAC (SEQ ID NO: 1386) | GGGCGGTGACGGAGCTGGGG (SEQ ID NO: 1271) |
| CTGTTCCAGTACTCCTCATC (SEQ ID NO: 1387) | GGCGGCCTGATGCCGAGTAC (SEQ ID NO: 1227) |
| GGAGTACTGGAACAGCCAGA (SEQ ID NO: 1388) | CTGTTCCAGTACTCGGCATC (SEQ ID NO: 1228) |
| GAACAGCCAGAAGGACTTCC (SEQ ID NO: 1389) | CTTCTGGCTGTTCCAGTACT (SEQ ID NO: 1066) |
| TGTCTTCCAGGAAGTCCTTC (SEQ ID NO: 1390) | CGAGTACTGGAACAGCCAGA (SEQ ID NO: 1067) |
| AGAAGGACTTCCTGGAAGAC (SEQ ID NO: 1391) | GAACAGCCAGAAGGACATCC (SEQ ID NO: 1068) |
| AGGACTTCCTGGAAGACAGG (SEQ ID NO: 1392) | CGTCTTCCAGGATGTCCTTC (SEQ ID NO: 1346) |
| GGACTTCCTGGAAGACAGGC (SEQ ID NO: 1393) | AGGACATCCTGGAAGACGAG (SEQ ID NO: 1347) |
| CCTGGAAGACAGGCGGGCCG (SEQ ID NO: 1394) | GGACATCCTGGAAGACGAGC (SEQ ID NO: 1348) |
| CCGCGGCCCGCCTGTCTTCC (SEQ ID NO: 1395) | CCTGGAAGACGAGCGGGCCG (SEQ ID NO: 1349) |
| GGAAGACAGGCGGGCCGCGG (SEQ ID NO: 1396) | CCGCGGCCCGCTCGTCTTCC (SEQ ID NO: 1350) |
| TCTGCAGTAGGTGTCCACCG (SEQ ID NO: 1281) | GGAAGACGAGCGGGCCGCGG (SEQ ID NO: 1351) |
| ACCTACTGCAGACACAACTA (SEQ ID NO: 1282) | TCTGCAGTAGGTGTCCACCG (SEQ ID NO: 1281) |
| CCTACTGCAGACACAACTAC (SEQ ID NO: 1283) | ACCTACTGCAGACACAACTA (SEQ ID NO: 1282) |
| CCCGTAGTTGTGTCTGCAGT (SEQ ID NO: 1284) | CCTACTGCAGACACAACTAC (SEQ ID NO: 1283) |
| CTACTGCAGACACAACTACG (SEQ ID NO: 1285) | CCCGTAGTTGTGTCTGCAGT (SEQ ID NO: 1284) |
| CAGACACAACTACGGGGTTG (SEQ ID NO: 1326) | CTACTGCAGACACAACTACG (SEQ ID NO: 1285) |
| TGGAGAGCTTCACAGTGCAG (SEQ ID NO: 1294) | CAGACACAACTACGGGGTTG (SEQ ID NO: 1326) |
| | TGGAGAGCTTCACAGTGCAG (SEQ ID NO: 1294) |

TABLE 5E
HLA-DRB1 13:02 and 14:01

| 13:02 | 14:01 |
|---|---|
| ATGGTGTGTCTGAGGCTCCC (SEQ ID NO: 1295) | ATGGTGTGTCTGAGGCTCCC (SEQ ID NO: 1295) |
| GTGTGTCTGAGGCTCCCTGG (SEQ ID NO: 1296) | GTGTGTCTGAGGCTCCCTGG (SEQ ID NO: 1296) |
| GCTCCCTGGAGGCTCCTGCA (SEQ ID NO: 1297) | GCTCCCTGGAGGCTCCTGCA (SEQ ID NO: 1297) |
| CTGCCATGCAGGAGCCTCCA (SEQ ID NO: 1298) | CTGCCATGCAGGAGCCTCCA (SEQ ID NO: 1298) |
| ACTGCCATGCAGGAGCCTCC (SEQ ID NO: 1299) | ACTGCCATGCAGGAGCCTCC (SEQ ID NO: 1299) |
| CACTGTCAGAACTGCCATGC (SEQ ID NO: 1300) | CACTGTCAGAACTGCCATGC (SEQ ID NO: 1300) |
| AGTTCTGACAGTGACACTGA (SEQ ID NO: 1301) | AGTTCTGACAGTGACACTGA (SEQ ID NO: 1301) |
| GATGGTGCTGAGCTCCCCAC (SEQ ID NO: 1245) | GATGGTGCTGAGCTCCCCAC (SEQ ID NO: 1245) |
| GCTGAGCTCCCCACTGGCTT (SEQ ID NO: 1246) | GCTGAGCTCCCCACTGGCTT (SEQ ID NO: 1246) |
| AGCTCCCCACTGGCTTTGGC (SEQ ID NO: 1247) | AGCTCCCCACTGGCTTTGGC (SEQ ID NO: 1247) |
| GCTCCCCACTGGCTTTGGCT (SEQ ID NO: 1248) | GCTCCCCACTGGCTTTGGCT (SEQ ID NO: 1248) |
| CTCCCCACTGGCTTTGGCTG (SEQ ID NO: 1249) | CTCCCCACTGGCTTTGGCTG (SEQ ID NO: 1249) |
| GTCCCCAGCCAAAGCCAGTG (SEQ ID NO: 1250) | GTCCCCAGCCAAAGCCAGTG (SEQ ID NO: 1250) |
| TGTCCCCAGCCAAAGCCAGT (SEQ ID NO: 1251) | TGTCCCCAGCCAAAGCCAGT (SEQ ID NO: 1251) |
| GTGTCCCCAGCCAAAGCCAG (SEQ ID NO: 1252) | GTGTCCCCAGCCAAAGCCAG (SEQ ID NO: 1252) |
| TCTGAGTGTCATTTCTTCAA (SEQ ID NO: 1302) | TCTGAGTGTCATTTCTTCAA (SEQ ID NO: 1302) |
| CTGAGTGTCATTTCTTCAAT (SEQ ID NO: 1303) | CTGAGTGTCATTTCTTCAAT (SEQ ID NO: 1303) |
| GTGTCATTTCTTCAATGGGA (SEQ ID NO: 1304) | GTGTCATTTCTTCAATGGGA (SEQ ID NO: 1304) |
| ATTTCTTCAATGGGACGGAG (SEQ ID NO: 1256) | ATTTCTTCAATGGGACGGAG (SEQ ID NO: 1256) |
| TTTCTTCAATGGGACGGAGC (SEQ ID NO: 1257) | TTTCTTCAATGGGACGGAGC (SEQ ID NO: 1257) |
| TCAATGGGACGGAGCGGGTG (SEQ ID NO: 1258) | TCAATGGGACGGAGCGGGTG (SEQ ID NO: 1258) |
| GACGGAGCGGGTGCGGTTCC (SEQ ID NO: 1339) | GACGGAGCGGGTGCGGTTCC (SEQ ID NO: 1339) |
| GGTTATGGAAGTATCTGTCC (SEQ ID NO: 1306) | GGTTATGGAAGTATCTGTCC (SEQ ID NO: 1306) |
| GGACAGATACTTCCATAACC (SEQ ID NO: 1307) | GGACAGATACTTCCATAACC (SEQ ID NO: 1307) |
| CAGATACTTCCATAACCAGG (SEQ ID NO: 1308) | CAGATACTTCCATAACCAGG (SEQ ID NO: 1308) |
| GCACGTTCTCCTCCTGGTTA (SEQ ID NO: 1309) | GCACGAACTCCTCCTGGTTA (SEQ ID NO: 1397) |
| CGAAGCGCACGTTCTCCTCC (SEQ ID NO: 1310) | CGAAGCGCACGAACTCCTCC (SEQ ID NO: 1359) |
| CGTGCGCTTCGACAGCGACG (SEQ ID NO: 1052) | CGTGCGCTTCGACAGCGACG (SEQ ID NO: 1052) |
| GTGCGCTTCGACAGCGACGT (SEQ ID NO: 1053) | GTGCGCTTCGACAGCGACGT (SEQ ID NO: 1053) |
| TGCGCTTCGACAGCGACGTG (SEQ ID NO: 1054) | TGCGCTTCGACAGCGACGTG (SEQ ID NO: 1054) |
| GCGCTTCGACAGCGACGTGG (SEQ ID NO: 1055) | GCGCTTCGACAGCGACGTGG (SEQ ID NO: 1055) |
| ACAGCGACGTGGGGGAGTTC (SEQ ID NO: 1056) | ACAGCGACGTGGGGGAGTAC (SEQ ID NO: 1263) |
| CAGCGACGTGGGGGAGTTCC (SEQ ID NO: 1057) | CAGCGACGTGGGGGAGTACC (SEQ ID NO: 1264) |
| CGACGTGGGGGAGTTCCGGG (SEQ ID NO: 1058) | CGACGTGGGGGAGTACCGGG (SEQ ID NO: 1265) |
| GGGGGAGTTCCGGGCGGTGA (SEQ ID NO: 1311) | GGGGGAGTACCGGGCGGTGA (SEQ ID NO: 1266) |
| GTTCCGGGCGGTGACGGAGC (SEQ ID NO: 1312) | GTACCGGGCGGTGACGGAGC (SEQ ID NO: 1267) |
| TTCCGGGCGGTGACGGAGCT (SEQ ID NO: 1313) | TACCGGGCGGTGACGGAGCT (SEQ ID NO: 1268) |
| TCCGGGCGGTGACGGAGCTG (SEQ ID NO: 1314) | ACCGGGCGGTGACGGAGCTG (SEQ ID NO: 1269) |
| GCCCCAGCTCCGTCACCGCC (SEQ ID NO: 1270) | GCCCCAGCTCCGTCACCGCC (SEQ ID NO: 1270) |
| GGGCGGTGACGGAGCTGGGG (SEQ ID NO: 1271) | GGGCGGTGACGGAGCTGGGG (SEQ ID NO: 1271) |
| GGCGGCCTGATGCCGAGTAC (SEQ ID NO: 1227) | GGAGCTGGGGCGGCCTGCTG (SEQ ID NO: 1398) |
| CTGTTCCAGTACTCGGCATC (SEQ ID NO: 1228) | GGCGGCCTGCTGCGGAGCAC (SEQ ID NO: 1399) |
| CTTCTGGCTGTTCCAGTACT (SEQ ID NO: 1066) | CTGTTCCAGTGCTCCGCAGC (SEQ ID NO: 1400) |
| CGAGTACTGGAACAGCCAGA (SEQ ID NO: 1067) | GGAGCACTGGAACAGCCAGA (SEQ ID NO: 1401) |
| GAACAGCCAGAAGGACATCC (SEQ ID NO: 1068) | GAACAGCCAGAAGGACCTCC (SEQ ID NO: 1272) |
| CGTCTTCCAGGATGTCCTTC (SEQ ID NO: 1346) | GCCAGAAGGACCTCCTGGAG (SEQ ID NO: 1402) |
| AGGACATCCTGGAAGACGAG (SEQ ID NO: 1347) | TCCGCTCCAGGAGGTCCTTC (SEQ ID NO: 1403) |
| GGACATCCTGGAAGACGAGC (SEQ ID NO: 1348) | AGAAGGACCTCCTGGAGCGG (SEQ ID NO: 1404) |
| CCTGGAAGACGAGCGGGCCG (SEQ ID NO: 1349) | AGGACCTCCTGGAGCGGAGG (SEQ ID NO: 1405) |
| CCGCGGCCCGCTCGTCTTCC (SEQ ID NO: 1350) | GGACCTCCTGGAGCGGAGGC (SEQ ID NO: 1406) |
| GGAAGACGAGCGGGCCGCGG (SEQ ID NO: 1351) | CGGCCCGCCTCCGCTCCAGG (SEQ ID NO: 1407) |
| TCTGCAGTAGGTGTCCACCG (SEQ ID NO: 1281) | CCTGGAGCGGAGGCGGGCCG (SEQ ID NO: 1408) |
| ACCTACTGCAGACACAACTA (SEQ ID NO: 1282) | CCTCGGCCCGCCTCCGCTCC (SEQ ID NO: 1409) |
| CCTACTGCAGACACAACTAC (SEQ ID NO: 1283) | GGAGCGGAGGCGGGCCGAGG (SEQ ID NO: 1410) |
| CCCGTAGTTGTGTCTGCAGT (SEQ ID NO: 1284) | TCTGCAATAGGTGTCCACCT (SEQ ID NO: 1411) |
| CTACTGCAGACACAACTACG (SEQ ID NO: 1285) | ACCTATTGCAGACACAACTA (SEQ ID NO: 1289) |
| TGCAGACACAACTACGGGGT (SEQ ID NO: 1286) | CCTATTGCAGACACAACTAC (SEQ ID NO: 1290) |
| GTGAGAGCTTCACAGTGCAG (SEQ ID NO: 1287) | CCCGTAGTTGTGTCTGCAAT (SEQ ID NO: 1291) |
| | CTATTGCAGACACAACTACG (SEQ ID NO: 1292) |
| | CAGACACAACTACGGGGTTG (SEQ ID NO: 1326) |
| | TGGAGAGCTTCACAGTGCAG (SEQ ID NO: 1294) |

TABLE 5F
HLA-DRB1 15:01 and 15:02

| 15:01 | 15:02 |
|---|---|
| ATGGTGTGTCTGAAGCTCCC (SEQ ID NO: 1239) | ATGGTGTGTCTGAAGCTCCC (SEQ ID NO: 1239) |
| GTGTGTCTGAAGCTCCCTGG (SEQ ID NO: 1240) | GTGTGTCTGAAGCTCCCTGG (SEQ ID NO: 1240) |
| CTGTCATGCAGGAGCCTCCA (SEQ ID NO: 1241) | CTGTCATGCAGGAGCCTCCA (SEQ ID NO: 1241) |
| GCTGTCATGCAGGAGCCTCC (SEQ ID NO: 1242) | GCTGTCATGCAGGAGCCTCC (SEQ ID NO: 1242) |
| CACTGTCAGCGCTGTCATGC (SEQ ID NO: 1243) | CACTGTCAGCGCTGTCATGC (SEQ ID NO: 1243) |
| AGCGCTGACAGTGACACTGA (SEQ ID NO: 1244) | AGCGCTGACAGTGACACTGA (SEQ ID NO: 1244) |
| GATGGTGCTGAGCTCCCCAC (SEQ ID NO: 1245) | GATGGTGCTGAGCTCCCCAC (SEQ ID NO: 1245) |
| AGCTCCCCACTGGCTTTGTC (SEQ ID NO: 1412) | AGCTCCCCACTGGCTTTGTC (SEQ ID NO: 1412) |
| GCTCCCCACTGGCTTTGTCT (SEQ ID NO: 1413) | GCTCCCCACTGGCTTTGTCT (SEQ ID NO: 1413) |
| CTCCCCACTGGCTTTGTCTG (SEQ ID NO: 1414) | CTCCCCACTGGCTTTGTCTG (SEQ ID NO: 1414) |
| GTCCCCAGACAAAGCCAGTG (SEQ ID NO: 1415) | GTCCCCAGACAAAGCCAGTG (SEQ ID NO: 1415) |
| TGTCCCCAGACAAAGCCAGT (SEQ ID NO: 1416) | TGTCCCCAGACAAAGCCAGT (SEQ ID NO: 1416) |
| GTGTCCCCAGACAAAGCCAG (SEQ ID NO: 1417) | GTGTCCCCAGACAAAGCCAG (SEQ ID NO: 1417) |
| GTTTCCTGTGGCAGCCTAAG (SEQ ID NO: 1418) | GTTTCCTGTGGCAGCCTAAG (SEQ ID NO: 1418) |
| TTTCCTGTGGCAGCCTAAGA (SEQ ID NO: 1419) | TTTCCTGTGGCAGCCTAAGA (SEQ ID NO: 1419) |
| ACTCCCTCTTAGGCTGCCAC (SEQ ID NO: 1420) | ACTCCCTCTTAGGCTGCCAC (SEQ ID NO: 1420) |
| AAGAAATGACACTCCCTCTT (SEQ ID NO: 1421) | AAGAAATGACACTCCCTCTT (SEQ ID NO: 1421) |
| AGGGAGTGTCATTTCTTCAA (SEQ ID NO: 1422) | AGGGAGTGTCATTTCTTCAA (SEQ ID NO: 1422) |
| GGGAGTGTCATTTCTTCAAT (SEQ ID NO: 1423) | GGGAGTGTCATTTCTTCAAT (SEQ ID NO: 1423) |
| GTGTCATTTCTTCAATGGGA (SEQ ID NO: 1304) | GTGTCATTTCTTCAATGGGA (SEQ ID NO: 1304) |
| ATTTCTTCAATGGGACGGAG (SEQ ID NO: 1256) | ATTTCTTCAATGGGACGGAG (SEQ ID NO: 1256) |
| TTTCTTCAATGGGACGGAGC (SEQ ID NO: 1257) | TTTCTTCAATGGGACGGAGC (SEQ ID NO: 1257) |
| TCAATGGGACGGAGCGGGTG (SEQ ID NO: 1258) | TCAATGGGACGGAGCGGGTG (SEQ ID NO: 1258) |
| GACGGAGCGGGTGCGGTTCC (SEQ ID NO: 1339) | GACGGAGCGGGTGCGGTTCC (SEQ ID NO: 1339) |
| GGTTATAGAAGTATCTGTCC (SEQ ID NO: 1383) | GGTTATAGAAGTATCTGTCC (SEQ ID NO: 1383) |
| GGACAGATACTTCTATAACC (SEQ ID NO: 1424) | GGACAGATACTTCTATAACC (SEQ ID NO: 1424) |
| CAGATACTTCTATAACCAGG (SEQ ID NO: 1425) | CAGATACTTCTATAACCAGG (SEQ ID NO: 1425) |
| CGAAGCGCACGGACTCCTCC (SEQ ID NO: 1426) | CGAAGCGCACGGACTCCTCC (SEQ ID NO: 1426) |
| CACGTCGCTGTCGAAGCGCA (SEQ ID NO: 1262) | CACGTCGCTGTCGAAGCGCA (SEQ ID NO: 1262) |
| CGTGCGCTTCGACAGCGACG (SEQ ID NO: 1052) | CGTGCGCTTCGACAGCGACG (SEQ ID NO: 1052) |
| GTGCGCTTCGACAGCGACGT (SEQ ID NO: 1053) | GTGCGCTTCGACAGCGACGT (SEQ ID NO: 1053) |
| TGCGCTTCGACAGCGACGTG (SEQ ID NO: 1054) | TGCGCTTCGACAGCGACGTG (SEQ ID NO: 1054) |
| GCGCTTCGACAGCGACGTGG (SEQ ID NO: 1055) | GCGCTTCGACAGCGACGTGG (SEQ ID NO: 1055) |
| ACAGCGACGTGGGGGAGTTC (SEQ ID NO: 1056) | ACAGCGACGTGGGGGAGTTC (SEQ ID NO: 1056) |
| CAGCGACGTGGGGGAGTTCC (SEQ ID NO: 1057) | CAGCGACGTGGGGGAGTTCC (SEQ ID NO: 1057) |
| CGACGTGGGGGAGTTCCGGG (SEQ ID NO: 1058) | CGACGTGGGGGAGTTCCGGG (SEQ ID NO: 1058) |
| GGGGGAGTTCCGGGCGGTGA (SEQ ID NO: 1311) | GGGGGAGTTCCGGGCGGTGA (SEQ ID NO: 1311) |
| GTTCCGGGCGGTGACGGAGC (SEQ ID NO: 1312) | GTTCCGGGCGGTGACGGAGC (SEQ ID NO: 1312) |
| TTCCGGGCGGTGACGGAGCT (SEQ ID NO: 1313) | TTCCGGGCGGTGACGGAGCT (SEQ ID NO: 1313) |
| TCCGGGCGGTGACGGAGCTG (SEQ ID NO: 1314) | TCCGGGCGGTGACGGAGCTG (SEQ ID NO: 1314) |
| GCCCCAGCTCCGTCACCGCC (SEQ ID NO: 1270) | GCCCCAGCTCCGTCACCGCC (SEQ ID NO: 1270) |
| GGGCGGTGACGGAGCTGGGG (SEQ ID NO: 1271) | GGGCGGTGACGGAGCTGGGG (SEQ ID NO: 1271) |
| GGCGGCCTGACGCTGAGTAC (SEQ ID NO: 1427) | GGCGGCCTGACGCTGAGTAC (SEQ ID NO: 1427) |
| CTGTTCCAGTACTCAGCGTC (SEQ ID NO: 1428) | CTGTTCCAGTACTCAGCGTC (SEQ ID NO: 1428) |
| TGAGTACTGGAACAGCCAGA (SEQ ID NO: 1429) | TGAGTACTGGAACAGCCAGA (SEQ ID NO: 1429) |
| GAACAGCCAGAAGGACATCC (SEQ ID NO: 1068) | GAACAGCCAGAAGGACATCC (SEQ ID NO: 1068) |
| CCAGAAGGACATCCTGGAGC (SEQ ID NO: 1430) | CCAGAAGGACATCCTGGAGC (SEQ ID NO: 1430) |
| CCTGCTCCAGGATGTCCTTC (SEQ ID NO: 1431) | CCTGCTCCAGGATGTCCTTC (SEQ ID NO: 1431) |
| AGGACATCCTGGAGCAGGCG (SEQ ID NO: 1432) | AGGACATCCTGGAGCAGGCG (SEQ ID NO: 1432) |
| GGACATCCTGGAGCAGGCGC (SEQ ID NO: 1433) | GGACATCCTGGAGCAGGCGC (SEQ ID NO: 1433) |
| CCTGGAGCAGGCGCGGGCCG (SEQ ID NO: 1434) | CCTGGAGCAGGCGCGGGCCG (SEQ ID NO: 1434) |
| CCGCGGCCCGCGCCTGCTCC (SEQ ID NO: 1435) | CCGCGGCCCGCGCCTGCTCC (SEQ ID NO: 1435) |
| GGAGCAGGCGCGGGCCGCGG (SEQ ID NO: 1436) | GGAGCAGGCGCGGGCCGCGG (SEQ ID NO: 1436) |
| TCTGCAGTAGGTGTCCACCG (SEQ ID NO: 1281) | TCTGCAGTAGGTGTCCACCG (SEQ ID NO: 1281) |
| ACCTACTGCAGACACAACTA (SEQ ID NO: 1282) | ACCTACTGCAGACACAACTA (SEQ ID NO: 1282) |
| CCTACTGCAGACACAACTAC (SEQ ID NO: 1283) | CCTACTGCAGACACAACTAC (SEQ ID NO: 1283) |
| CCCGTAGTTGTGTCTGCAGT (SEQ ID NO: 1284) | CCCGTAGTTGTGTCTGCAGT (SEQ ID NO: 1284) |
| CTACTGCAGACACAACTACG (SEQ ID NO: 1285) | CTACTGCAGACACAACTACG (SEQ ID NO: 1285) |
| CAGACACAACTACGGGGTTG (SEQ ID NO: 1326) | TGCAGACACAACTACGGGGT (SEQ ID NO: 1286) |
| TGGAGAGCTTCACAGTGCAG (SEQ ID NO: 1294) | GTGAGAGCTTCACAGTGCAG (SEQ ID NO: 1287) |

TABLE 6
HLA-A 01:01, 03:01, 29:02, 33:01

| A 01:01 | A 03:01 | A 29:02 | A 33:01 |
|---|---|---|---|
| CGTAGCCCACGGCGATG | ACAGCGACGCCGCGAGC | CGTAGCCCACGGCGATG | CGTAGCCCACGGCGATG |
| AAG (SEQ ID NO: 56) | CAG (SEQ ID NO: 59) | AAG (SEQ ID NO: 56) | AAG (SEQ ID NO: 56) |
| ATTTCTTCACATCCGTGT | CGACGCCGCGAGCCAGA | ACAGCGACGCCGCGAGC | AGTATTGGGACCGGAAC |
| CC (SEQ ID NO: 7) | GGA (SEQ ID NO: 61) | CAG (SEQ ID NO: 59) | ACA (SEQ ID NO: 156) |
| GTCTCCTGGTCCCAATAC | ATTTCTTCACATCCGTGT | ACAGACTGACCGAGCGA | CCGGAACACACGGAATG |
| TC (SEQ ID NO: 91) | CC (SEQ ID NO: 7) | ACC (SEQ ID NO: 103) | TGA (SEQ ID NO: 157) |
| GTAGCCCACGGCGATGA | ACAGACTGACCGAGTGG | CAGACTGACCGAGCGAA | ACAGCGACGCCGCGAGC |
| AGC (SEQ ID NO: 54) | ACC (SEQ ID NO: 131) | CCT (SEQ ID NO: 105) | CAG (SEQ ID NO: 59) |
| TAGCCCACGGCGATGAA | TTCACATCCGTGTCCCGG | CGTGTCGTCCACGTAGC | GTGTTCCGGTCCCAATA |
| GCG (SEQ ID NO: 52) | CC (SEQ ID NO: 10) | CCA (SEQ ID NO: 60) | CTC (SEQ ID NO: 155) |
| ACAGACTGACCGAGCGA | CAGACTGACCGAGTGGA | AGACTGACCGAGCGAAC | GTAGCCCACGGCGATGA |
| ACC (SEQ ID NO: 103) | CCT (SEQ ID NO: 132) | CTG (SEQ ID NO: 107) | AGC (SEQ ID NO: 54) |
| CAGACTGACCGAGCGAA | CCACTCGGTCAGTCTGTG | TGGACGACACGCAGTTC | TAGCCCACGGCGATGAA |
| CCT (SEQ ID NO: 105) | AC (SEQ ID NO: 130) | GTG (SEQ ID NO: 57) | GCG (SEQ ID NO: 52) |
| AGACTGACCGAGCGAAC | CCTTCACATTCCGTGTCT | GTAGCCCACGGCGATGA | CCACTCGGTCAATCTGT |
| CTG (SEQ ID NO: 107) | CC (SEQ ID NO: 127) | AGC (SEQ ID NO: 54) | GAG (SEQ ID NO: 204) |
| GAGCCCCGCTTCATCGC | CGGCTCCATCCTCTGGCT | CGCTCGGTCAGTCTGTG | GAGCCCCGCTTCATCGC |
| CGT (SEQ ID NO: 49) | CG (SEQ ID NO: 62) | ACT (SEQ ID NO: 196) | CGT (SEQ ID NO: 49) |
| CGTGTCGTCCACGTAGC | AGTAGCCGCGCAGGGTC | TAGCCCACGGCGATGAA | CCACTCACAGATTGACC |
| CCA (SEQ ID NO: 60) | CCC (SEQ ID NO: 112) | GCG (SEQ ID NO: 52) | GAG (SEQ ID NO: 202) |
| GGACCTGCGCTCTTGGA | CCAGGAGACACGGAATG | | |
| CCG (SEQ ID NO: 1437) | TGA (SEQ ID NO: 126) | | |
| TGCCGTCGTAGGCGTCC | | | |
| TGC (SEQ ID NO: 1438) | | | |

TABLE 7
HLA-A 11:01 and 26:01

| A 11:01 | A 26:01 |
|---|---|
| ACAGCGACGCCGCGAGCCAG (SEQ ID NO: 59) | ATTTCTACACCTCCGTGTCC (SEQ ID NO: 117) |
| CGACGCCGCGAGCCAGAGGA (SEQ ID NO: 61) | GGAGGTGTAGAAATACCTCA (SEQ ID NO: 116) |
| ACAGACTGACCGAGTGGACC (SEQ ID NO: 131) | TCGACAGCGACGCCGCGAGC (SEQ ID NO: 120) |
| CAGACTGACCGAGTGGACCT (SEQ ID NO: 132) | CGTAGCCCACTGCGATGAAG (SEQ ID NO: 53) |
| CCACTCGGTCAGTCTGTGAC (SEQ ID NO: 130) | TGGACGACACGCAGTTCGTG (SEQ ID NO: 57) |
| ATTTCTACACCTCCGTGTCC (SEQ ID NO: 117) | TAGCCCACTGCGATGAAGCG (SEQ ID NO: 47) |
| CCTTCACATTCCGTGTCTCC (SEQ ID NO: 127) | AGACTCACCGAGTGGACCTG (SEQ ID NO: 106) |
| CGGCTCCATCCTCTGGCTCG (SEQ ID NO: 62) | GAGCCCCGCTTCATCGCAGT (SEQ ID NO: 43) |
| AGTAGCCGCGCAGGGTCCCC (SEQ ID NO: 112) | ACAGCGACGCCGCGAGCCGG (SEQ ID NO: 121) |
| TACACCTCCGTGTCCCGGCC (SEQ ID NO: 118) | GGGTCCGGAGTATTGGGACG (SEQ ID NO: 88) |
| CCAGGAGACACGGAATGTGA (SEQ ID NO: 126) | |

TABLE 8
HLA-DQB1 02:01, 06:02, 03:03, and 05:01

| A 02:01 | A 06:02 | A 03:03 | A 05:01 |
|---|---|---|---|
| CGAGTACTGGAACAGCC | CAGATACATCTATAACC | CAGATACATCTATAACC | CGAGTACTGGAACAGCC |
| AGA (SEQ ID NO: 1067) | GAG (SEQ ID NO: 1094) | GAG (SEQ ID NO: 1094) | AGA (SEQ ID NO: 1067) |
| AGGTCGTGCGGAGCTCC | CTCTCCTCTGCAAGATCC | CGAGTACTGGAACAGCC | TCCCGTTGGTGAAGTAG |
| AAC (SEQ ID NO: 1080) | CG (SEQ ID NO: 1238) | AGA (SEQ ID NO: 1067) | CAC (SEQ ID NO: 1164) |
| CTGGCTGTTCCAGTACTC | GATTTCGTGTTCCAGTTT | GTGCTACTTCACCAACG | AACTACGAGGTGGCGTA |
| GG (SEQ ID NO: 1065) | AA (SEQ ID NO: 1215) | GGA (SEQ ID NO: 1091) | CCG (SEQ ID NO: 1197) |
| GGCATGTGCTACTTCACC | CGAGTACTGGAACAGCC | AGGTCGTGCGGAGCTCC | GTGCTACTTCACCAACG |
| AA (SEQ ID NO: 1048) | AGA (SEQ ID NO: 1067) | AAC (SEQ ID NO: 1080) | GGA (SEQ ID NO: 1091) |
| GCATGTGCTACTTCACCA | ACTACGAGGTGGCGTTC | GGCATGTGCTACTTCAC | GGTGTGCAGACACAACT |
| AC (SEQ ID NO: 1049) | CGC (SEQ ID NO: 1236) | CAA (SEQ ID NO: 1048) | ACG (SEQ ID NO: 1195) |
| CGAAGCGCACGATCTCTT | AACTACGAGGTGGCGTT | CACCGTGTCCAACTCCG | GTGCAGACACAACTACG |
| CT (SEQ ID NO: 1051) | CCG (SEQ ID NO: 1235) | CCC (SEQ ID NO: 1118) | AGG (SEQ ID NO: 1196) |
| AGCTCCGCACGACCTTGC | GTGCTACTTCACCAACG | GCATGTGCTACTTCACC | ACTACGAGGTGGCGTAC |
| AG (SEQ ID NO: 1081) | GGA (SEQ ID NO: 1091) | AAC (SEQ ID NO: 1049) | CGC (SEQ ID NO: 1198) |
| ACGCACGCGCTCTGTCCC | GGTGTGCAGACACAACT | CGCACGCTTCGACAGCG | ACAGCGACGTGGGGGT |
| GT (SEQ ID NO: 1050) | ACG (SEQ ID NO: 1195) | ACG (SEQ ID NO: 1096) | GTAC (SEQ ID NO: 1172) |
| CGTGCGCTTCGACAGCG | GTGCAGACACAACTACG | ACACCGTGTCCAACTCC | CCAACGGGACGGAGCGC |
| ACG (SEQ ID NO: 1052) | AGG (SEQ ID NO: 1196) | GCC (SEQ ID NO: 1119) | GTG (SEQ ID NO: 1165) |
| ACAGCGACGTGGGGGA | TACCGCGCGGTGACGCC | ACGCACGCGCTCCGTCC | CGAAGCGCACGTACTCC |
| GTTC (SEQ ID NO: 1056) | GCA (SEQ ID NO: 1222) | CGT (SEQ ID NO: 1092) | TCT (SEQ ID NO: 1171) |
| AAGGGGGGTGGACACA | CAGATACATCTATAACC | | |
| ACGC (SEQ ID NO: 1439) | GAG (SEQ ID NO: 1094) | | |
| CCGGTGGTTTCGGAATG | | | |
| ACC (SEQ ID NO: 1440) | | | |

TABLE 9
HLA-DQB1 06:01 and 06:03

| DQB1 06:01 | DQB1 06:03 |
|---|---|
| CAGATACATCTATAACCGAG (SEQ ID NO: 1094) | CTCTCCTCTGCAAGATCCCG (SEQ ID NO: 1238) |
| CTCTCCTCTGCAAGATCCCG (SEQ ID NO: 1238) | CGAGTACTGGAACAGCCAGA (SEQ ID NO: 1067) |
| CGAGTACTGGAACAGCCAGA (SEQ ID NO: 1067) | ACTACGAGGTGGCGTTCCGC (SEQ ID NO: 1236) |
| GGTGTGCAGACACAACTACG (SEQ ID NO: 1195) | GTGCTACTTCACCAACGGGA (SEQ ID NO: 1091) |
| AACTACGAGGTGGCGTTCCG (SEQ ID NO: 1235) | AACTACGAGGTGGCGTTCCG (SEQ ID NO: 1235) |
| ACTACGAGGTGGCGTTCCGC (SEQ ID NO: 1236) | GGTGTGCAGACACAACTACG (SEQ ID NO: 1195) |
| ATACATCTATAACCGAGAGG (SEQ ID NO: 1441) | GTGCAGACACAACTACGAGG (SEQ ID NO: 1196) |
| GTGCAGACACAACTACGAGG (SEQ ID NO: 1196) | GGCATGTGCTACTTCACCAA (SEQ ID NO: 1048) |
| ACGCACGCGCTCCGTCCCAT (SEQ ID NO: 1442) | TACCGCGCGGTGACGCCGCA (SEQ ID NO: 1222) |
| ACACCGTGTCCAACTCCGCT (SEQ ID NO: 1443) | GCATGTGCTACTTCACCAAC (SEQ ID NO: 1049) |

TABLE 10
HLA-A 24:02

| Exon 1 | Exon 2 |
|---|---|
| GAGGGTTCGGGGCGCCATGA (SEQ ID NO: 1) | GGAGAAATACCTCATGGAGT (SEQ ID NO: 135) |
| AGTAGCAGGACGAGGGTTCG (SEQ ID NO: 6) | TGGAGAAATACCTCATGGAG (SEQ ID NO: 137) |
| GAGTAGCAGGACGAGGGTTC (SEQ ID NO: 9) | GGATGTGGAGAAATACCTCA (SEQ ID NO: 138) |
| AGAGTAGCAGGACGAGGGTT (SEQ ID NO: 12) | ATTTCTCCACATCCGTGTCC (SEQ ID NO: 139) |
| AACCCTCGTCCTGCTACTCT (SEQ ID NO: 15) | TCCACATCCGTGTCCCGGCC (SEQ ID NO: 140) |
| ACCCTCGTCCTGCTACTCTC (SEQ ID NO: 18) | GCCGGGCCGGGACACGGATG (SEQ ID NO: 141) |
| CCCTCGTCCTGCTACTCTCG (SEQ ID NO: 21) | TCCGTGTCCCGGCCCGGCCG (SEQ ID NO: 13) |
| CCCCGAGAGTAGCAGGACGA (SEQ ID NO: 24) | CCGTGTCCCGGCCCGGCCGC (SEQ ID NO: 16) |
| CCTCGTCCTGCTACTCTCGG (SEQ ID NO: 27) | CCCGCGGCCGGGCCGGGACA (SEQ ID NO: 19) |
| CCCCCGAGAGTAGCAGGACG (SEQ ID NO: 30) | CGTGTCCCGGCCCGGCCGCG (SEQ ID NO: 22) |
| CCTGCTACTCTCGGGGGCCC (SEQ ID NO: 35) | GGGCTCCCCGCGGCCGGGCC (SEQ ID NO: 25) |
| CCAGGGCCCCCGAGAGTAGC (SEQ ID NO: 38) | GGGGCTCCCCGCGGCCGGGC (SEQ ID NO: 28) |
| CCCTGGCCCTGACCCAGACC (SEQ ID NO: 41) | AAGCGGGGCTCCCCGCGGCC (SEQ ID NO: 31) |
| CCAGGTCTGGGTCAGGGCCA (SEQ ID NO: 44) | GAAGCGGGGCTCCCCGCGGC (SEQ ID NO: 34) |
| CCTGGCCCTGACCCAGACCT (SEQ ID NO: 48) | CGATGAAGCGGGGCTCCCCG (SEQ ID NO: 37) |
| CCCAGGTCTGGGTCAGGGCC (SEQ ID NO: 51) | GGAGCCCCGCTTCATCGCCG (SEQ ID NO: 45) |
| | GAGCCCCGCTTCATCGCCGT (SEQ ID NO: 49) |
| | TAGCCCACGGCGATGAAGCG (SEQ ID NO: 52) |
| | GTAGCCCACGGCGATGAAGC (SEQ ID NO: 54) |
| | CGTAGCCCACGGCGATGAAG (SEQ ID NO: 56) |
| | CTTCATCGCCGTGGGCTACG (SEQ ID NO: 58) |
| | CGTGTCGTCCACGTAGCCCA (SEQ ID NO: 60) |
| | TGGACGACACGCAGTTCGTG (SEQ ID NO: 57) |
| | ACAGCGACGCCGCGAGCCAG (SEQ ID NO: 59) |
| | CGACGCCGCGAGCCAGAGGA (SEQ ID NO: 61) |
| | CGGCTCCATCCTCTGGCTCG (SEQ ID NO: 62) |
| | CGAGCCAGAGGATGGAGCCG (SEQ ID NO: 64) |
| | GAGCCAGAGGATGGAGCCGC (SEQ ID NO: 66) |
| | GCGCCCGCGGCTCCATCCTC (SEQ ID NO: 68) |
| | GGATGGAGCCGCGGGCGCCG (SEQ ID NO: 70) |
| | TGCTCTATCCACGGCGCCCG (SEQ ID NO: 72) |
| | GCGGGCGCCGTGGATAGAGC (SEQ ID NO: 74) |
| | GGCGCCGTGGATAGAGCAGG (SEQ ID NO: 75) |
| | GCGCCGTGGATAGAGCAGGA (SEQ ID NO: 76) |
| | CGCCGTGGATAGAGCAGGAG (SEQ ID NO: 79) |
| | GGCCCCTCCTGCTCTATCCA (SEQ ID NO: 81) |
| | GTGGATAGAGCAGGAGGGGC (SEQ ID NO: 83) |
| | AGCAGGAGGGGCCGGAGTAT (SEQ ID NO: 85) |
| | GCAGGAGGGGCCGGAGTATT (SEQ ID NO: 87) |
| | GGGGCCGGAGTATTGGGACG (SEQ ID NO: 142) |
| | GTCTCCTCGTCCCAATACTC (SEQ ID NO: 143) |
| | GAGTATTGGGACGAGGAGAC (SEQ ID NO: 144) |
| | AGTATTGGGACGAGGAGACA (SEQ ID NO: 145) |
| | CGAGGAGACAGGGAAAGTGA (SEQ ID NO: 146) |
| | CTCTCGGTCAGTCTGTGAGT (SEQ ID NO: 147) |
| | TCTCTCGGTCAGTCTGTGAG (SEQ ID NO: 148) |
| | AGACTGACCGAGAGAACCTG (SEQ ID NO: 149) |
| | GCGCGATCCGCAGGTTCTCT (SEQ ID NO: 150) |
| | AGTAGCGGAGCGCGATCCGC (SEQ ID NO: 151) |
| | CCGCTACTACAACCAGAGCG (SEQ ID NO: 152) |
| | CCTCGCTCTGGTTGTAGTAG (SEQ ID NO: 153) |

TABLE 11
HLA-B 07:02, 44:03, 57:01, and 08:01

| 07:02 | 44:03 | 57:01 | 08:01 |
|---|---|---|---|
| TGGACGACACCCAGTTC | CGTAGCCCACGGTGATG | CGCTGTCGAACCTCACG | TGTCGAAATACCTCATG |
| GTG (SEQ ID NO: 238) | AAG (SEQ ID NO: 280) | AAC (SEQ ID NO: 240) | GAG (SEQ ID NO: 221) |
| GCTGTCGAACCTCACGA | GTCTCCCGGTCCCAATA | GCTGTCGAACCTCACGA | GTCGAAATACCTCATGG |
| ACT (SEQ ID NO: 239) | CTC (SEQ ID NO: 298) | ACT (SEQ ID NO: 239) | AGT (SEQ ID NO: 220) |
| CGCTGTCGAACCTCACG | ACAGCGACGCCACGAGT | TGGACGACACCCAGTTC | GGCGGTGTCGAAATACC |
| AAC (SEQ ID NO: 240) | CCG (SEQ ID NO: 283) | GTG (SEQ ID NO: 238) | TCA (SEQ ID NO: 222) |
| ATTTCTACACCTCCGTGT | TAGCCCACGGTGATGAA | CGTAGCCCACTGCGATG | CGTAGCCCACTGAGATG |
| CC (SEQ ID NO: 117) | GCG (SEQ ID NO: 278) | AAG (SEQ ID NO: 53) | AAG (SEQ ID NO: 236) |
| CACACAGATCTACAAGG | GAGCCCCGCTTCATCAC | ACAGCGACGCCGCGAGT | TAGCCCACTGAGATGAA |
| CCC (SEQ ID NO: 249) | CGT (SEQ ID NO: 277) | CCG (SEQ ID NO: 318) | GCG (SEQ ID NO: 234) |
| GGAGGTGTAGAAATACC | CGTGTCGTCCACGTAGC | CTCTCGGTAAGTCTGCG | CGGCTCCTCTCTCGGACT |
| TCA (SEQ ID NO: 116) | CCA (SEQ ID NO: 60) | CGG (SEQ ID NO: 1027) | CG (SEQ ID NO: 242) |
| CGTAGCCCACTGAGATG | CGACGCCACGAGTCCGA | CCTCGCTCTGGTTGTAGT | ATTTCGACACCGCCATGT |
| AAG (SEQ ID NO: 236) | GGA (SEQ ID NO: 284) | AG (SEQ ID NO: 153) | CC (SEQ ID NO: 223) |
| TAGCCCACTGAGATGAA | CTGGTTGTAGTAGCGGA | GTTCTCTCGGTAAGTCTG | GTGTTCCGGTCCCAATA |
| GCG (SEQ ID NO: 234) | GCG (SEQ ID NO: 304) | CG (SEQ ID NO: 1028) | CTC (SEQ ID NO: 155) |
| CGGCTCCTCTCTCGGACT | CCTCGCTCTGGTTGTAGT | CGACGCCGCGAGTCCGA | CGACGCCGCGAGTCCGA |
| CG (SEQ ID NO: 242) | AG (SEQ ID NO: 153) | GGA (SEQ ID NO: 319) | GAG (SEQ ID NO: 241) |
| GTGTTCCGGTCCCAATAC | CCGCTACTACAACCAGA | TAGCCCACTGCGATGAA | GTAGCCCACTGAGATGA |
| TC (SEQ ID NO: 155) | GCG (SEQ ID NO: 152) | GCG (SEQ ID NO: 47) | AGC (SEQ ID NO: 235) |
| GCTGTCGAACCTCACGA | | | |
| ACT (SEQ ID NO: 239) | | | |
| CGCCGTGGATAGAGCAG | | | |
| GAG (SEQ ID NO: 79) | | | |

TABLE 12
HLA-B 14:02, 50:01, 37:01, and 52:01

| B 14:02 | B 50:01 | B 37:01 | B 52:01 |
|---|---|---|---|
| GTGTTCCGGTCCCAATAT | CGTAGCCCACGGTGATG | GCTGTCGAACCTCACGA | CGCTGTCGAACCTCACG |
| TC (SEQ ID NO: 311) | AAG (SEQ ID NO: 280) | ACT (SEQ ID NO: 239) | AAC (SEQ ID NO: 240) |
| TAGCCCACTGAGATGAA | GTCTCCCGGTCCCAATAC | TGGACGACACCCAGTTC | GCTGTCGAACCTCACGA |
| GCG (SEQ ID NO: 234) | TC (SEQ ID NO: 298) | GTG (SEQ ID NO: 238) | ACT (SEQ ID NO: 239) |
| CGTAGCCCACTGAGATG | ACAGCGACGCCACGAGT | CGCTGTCGAACCTCACG | TGGACGACACCCAGTTC |
| AAG (SEQ ID NO: 236) | CCG (SEQ ID NO: 283) | AAC (SEQ ID NO: 240) | GTG (SEQ ID NO: 238) |
| CGGCTCCTCTCTCGGACT | TAGCCCACGGTGATGAA | CGTAGCCCACTGAGATG | CGTAGCCCACTGCGATG |
| CG (SEQ ID NO: 242) | GCG (SEQ ID NO: 278) | AAG (SEQ ID NO: 236) | AAG (SEQ ID NO: 53) |
| ATTTCTACACCGCCGTGT | CGTGTCGTCCACGTAGC | TAGCCCACTGAGATGAA | GTCTCCCGGTCCCAATAC |
| CC (SEQ ID NO: 305) | CCA (SEQ ID NO: 60) | GCG (SEQ ID NO: 234) | TC (SEQ ID NO: 298) |
| CGACGCCGCGAGTCCGA | GAGCCCCGCTTCATCAC | ACAGCGACGCCGCGAGT | ACAGCGACGCCGCGAGT |
| GAG (SEQ ID NO: 241) | CGT (SEQ ID NO: 277) | CCG (SEQ ID NO: 318) | CCG (SEQ ID NO: 318) |
| GTAGCCCACTGAGATGA | CGACGCCACGAGTCCGA | CTGGTTGTAGTAGCGGA | CCTCGCTCTGGTTGTAGT |
| AGC (SEQ ID NO: 235) | GGA (SEQ ID NO: 284) | GCA (SEQ ID NO: 1444) | AG (SEQ ID NO: 153) |
| GAGCCCCGCTTCATCTCA | TGGACGACACGCTGTTC | GTCTCCCGGTCCCAATA | TAGCCCACTGCGATGAA |
| GT (SEQ ID NO: 233) | GTG (SEQ ID NO: 1018) | CTC (SEQ ID NO: 298) | GCG (SEQ ID NO: 47) |
| GAGAGCCTGCGGAACCT | GTAGCCCACGGTGATGA | TCTGGTTGTAGTAGCGG | CGACGCCGCGAGTCCGA |
| GCG (SEQ ID NO: 253) | AGC (SEQ ID NO: 279) | AGC (SEQ ID NO: 1445) | GGA (SEQ ID NO: 319) |
| TGGACGACACGCAGTTC | GAGAGCCTGCGGAACCT | GTAGCCCACTGAGATGA | CCGCTACTACAACCAGA |
| GTG (SEQ ID NO: 57) | GCG (SEQ ID NO: 253) | AGC (SEQ ID NO: 235) | GCG (SEQ ID NO: 152) |

TABLE 13
HLA-B 38:01 and 35:01

| B 38:01 | B 35:01 |
|---|---|
| ATTTCTACACCTCCGTGTCC (SEQ ID NO: 117) | GCTGTCGAACCTCACGAACT (SEQ ID NO: 239) |
| GGAGGTGTAGAAATACCTCA (SEQ ID NO: 116) | TGGACGACACCCAGTTCGTG (SEQ ID NO: 238) |
| TAGCCCACTGAGATGAAGCG (SEQ ID NO: 234) | CGCTGTCGAACCTCACGAAC (SEQ ID NO: 240) |
| CGGCTCCTCTCTCGGACTCG (SEQ ID NO: 242) | CGTAGCCCACTGCGATGAAG (SEQ ID NO: 53) |
| GTGTTCCGGTCCCAATATTC (SEQ ID NO: 311) | GTGTTCCGGTCCCAATACTC (SEQ ID NO: 155) |
| CGTAGCCCACTGAGATGAAG (SEQ ID NO: 236) | ACAGCGACGCCGCGAGTCCG (SEQ ID NO: 318) |
| CGACGCCGCGAGTCCGAGAG (SEQ ID NO: 241) | GAGAGCCTGCGGAACCTGCG (SEQ ID NO: 253) |
| CCGCTACTACAACCAGAGCG (SEQ ID NO: 152) | TAGCCCACTGCGATGAAGCG (SEQ ID NO: 47) |
| GTAGCCCACTGAGATGAAGC (SEQ ID NO: 235) | CGACGCCGCGAGTCCGAGGA (SEQ ID NO: 319) |
| GAGCCCCGCTTCATCTCAGT (SEQ ID NO: 233) | GAGCCCCGCTTCATCGCAGT (SEQ ID NO: 43) |

TABLE 14
HLA-DRB1 03:01, 15:01, 07:01, and 01:02

| DRB1 03:01 | DRB1 15:01 | DRB1 07:01 | DRB1 01:02 |
|---|---|---|---|
| TTTCTTCAATGGGACGG | CGAAGCGCACGGACTC | TGCAGACACAACTACGG | TTTCTTCAATGGGACGGA |
| AGC (SEQ ID NO: 1257) | CTCC (SEQ ID NO: | GGT (SEQ ID NO: 1286) | GC (SEQ ID NO: 1257) |
| | 1426) | | |
| CAGACACAACTACGGGG | TTTCTTCAATGGGACGG | ATTTCTTCAACGGGACG | ATTTCTTCAATGGGACGG |
| TTG (SEQ ID NO: 1326) | AGC (SEQ ID NO: 1257) | GAG (SEQ ID NO: 1336) | AG (SEQ ID NO: 1256) |
| ATTTCTTCAATGGGACG | CAGACACAACTACGGG | TTTCTTCAACGGGACGG | CGAGTACTGGAACAGCC |
| GAG (SEQ ID NO: 1256) | GTTG (SEQ ID NO: | AGC (SEQ ID NO: 1337) | AGA (SEQ ID NO: 1067) |
| | 1326) | | |
| GTGTCATTTCTTCAATGG | GGACAGATACTTCTATA | GTGTCATTTCTTCAACGG | TCTGCAATAGGTGTCCAC |
| GA (SEQ ID NO: 1304) | ACC (SEQ ID NO: 1424) | GA (SEQ ID NO: 1335) | CG (SEQ ID NO: 1288) |
| CGAGTACTGGAACAGCC | GGGAGTGTCATTTCTTC | ACAGCGACGTGGGGGA | CGAAGCGCACGGACTCC |
| AGA (SEQ ID NO: 1067) | AAT (SEQ ID NO: 1423) | GTAC (SEQ ID NO: 1263) | TCT (SEQ ID NO: 1261) |
| CTGTTCCAGTACTCGGC | ATTTCTTCAATGGGACG | ACCGTGTGCAGACACAA | CTGTTCCAGTACTCGGCA |
| ATC (SEQ ID NO: 1228) | GAG (SEQ ID NO: 1256) | CTA (SEQ ID NO: 1379) | TC (SEQ ID NO: 1228) |
| CTACTGCAGACACAACT | GGTTATAGAAGTATCTG | CGAAGCGCACGAACTCC | ACAGCGACGTGGGGGA |
| ACG (SEQ ID NO: 1285) | TCC (SEQ ID NO: 1383) | TCC (SEQ ID NO: 1359) | GTAC (SEQ ID NO: 1263) |
| CGTGCGCTTCGACAGCG | CAGATACTTCTATAACC | CGTGTGCAGACACAACT | CACGTCGCTGTCGAAGC |
| ACG (SEQ ID NO: 1052) | AGG (SEQ ID NO: 1425) | ACG (SEQ ID NO: 1382) | GCA (SEQ ID NO: 1262) |
| ACAGCGACGTGGGGGA | AGGGAGTGTCATTTCTT | CGACGTGGGGGAGTACC | CGACGTGGGGGAGTACC |
| GTTC (SEQ ID NO: 1056) | CAA (SEQ ID NO: 1422) | GGG (SEQ ID NO: 1265) | GGG (SEQ ID NO: 1265) |
| GTGCGCTTCGACAGCGA | ACTCCCTCTTAGGCTGC | CTGTTCCAGGACTCGGC | CGTGCGCTTCGACAGCG |
| CGT (SEQ ID NO: 1053) | CAC (SEQ ID NO: 1420) | GAC (SEQ ID NO: 1364) | ACG (SEQ ID NO: 1052) |

TABLE 15
HLA-DRB1 10:01, 15:02, 13:01, and 01:01

| DRB1 10:01 | DRB1 15:02 | DRB1 13:01 | DRB1 01:01 |
|---|---|---|---|
| TTTCTTCAACGGGACGG | TGCAGACACAACTACGG | TTTCTTCAATGGGACGG | TGCAGACACAACTACGG |
| AGC (SEQ ID NO: 1337) | GGT (SEQ ID NO: 1286) | AGC (SEQ ID NO: 1257) | GGT (SEQ ID NO: 1286) |
| TGCAGACACAACTACGG | CGAAGCGCACGGACTCC | CAGACACAACTACGGGG | TTTCTTCAATGGGACGGA |
| GGT (SEQ ID NO: 1286) | TCC (SEQ ID NO: 1426) | TTG (SEQ ID NO: 1326) | GC (SEQ ID NO: 1257) |
| ATTTCTTCAACGGGACG | TTTCTTCAATGGGACGG | ATTTCTTCAATGGGACG | ATTTCTTCAATGGGACGG |
| GAG (SEQ ID NO: 1336) | AGC (SEQ ID NO: 1257) | GAG (SEQ ID NO: 1256) | AG (SEQ ID NO: 1256) |
| GTGTCATTTCTTCAACGG | GGACAGATACTTCTATA | GTGTCATTTCTTCAATGG | CGAGTACTGGAACAGCC |
| GA (SEQ ID NO: 1335) | ACC (SEQ ID NO: 1424) | GA (SEQ ID NO: 1304) | AGA (SEQ ID NO: 1067) |
| CGAGTACTGGAACAGCC | GGGAGTGTCATTTCTTC | CGAGTACTGGAACAGCC | TCTGCAGTAGGTGTCCAC |
| AGA (SEQ ID NO: 1067) | AAT (SEQ ID NO: 1423) | AGA (SEQ ID NO: 1067) | CG (SEQ ID NO: 1281) |
| TCTGCAGTAGGTGTCCA | ATTTCTTCAATGGGACG | TCTGCAGTAGGTGTCCA | CGAAGCGCACGGACTCC |
| CCG (SEQ ID NO: 1281) | GAG (SEQ ID NO: 1256) | CCG (SEQ ID NO: 1281) | TCT (SEQ ID NO: 1261) |
| CGCGCTACGACAGCGAC | GGTTATAGAAGTATCTG | CTGTTCCAGTACTCGGC | ACAGCGACGTGGGGGA |
| GTG (SEQ ID NO: 1446) | TCC (SEQ ID NO: 1383) | ATC (SEQ ID NO: 1228) | GTAC (SEQ ID NO: 1263) |
| AAGACGCGTCCATAACC | CAGATACTTCTATAACC | CTACTGCAGACACAACT | CTGTTCCAGTACTCGGCA |
| AAG (SEQ ID NO: 1447) | AGG (SEQ ID NO: 1425) | ACG (SEQ ID NO: 1285) | TC (SEQ ID NO: 1228) |
| GCGCTACGACAGCGACG | AGGGAGTGTCATTTCTT | ACCTACTGCAGACACAA | CACGTCGCTGTCGAAGC |
| TGG (SEQ ID NO: 1448) | CAA (SEQ ID NO: 1422) | CTA (SEQ ID NO: 1282) | GCA (SEQ ID NO: 1262) |
| ACAGCGACGTGGGGGA | ACTCCCTCTTAGGCTGC | CGTGCGCTTCGACAGCG | CGACGTGGGGGAGTACC |
| GTAC (SEQ ID NO: 1263) | CAC (SEQ ID NO: 1420) | ACG (SEQ ID NO: 1052) | GGG (SEQ ID NO: 1265) |

TABLE 16
HLA-DPB1 01:01, 02:01, 03:01, and 04:01

| DPB1*01:01 | DPB1*02:01 | DPB1*03:01 | DPB1*04:01 |
|---|---|---|---|
| GAGATACATCTACAACC | GACACAACTACGAGCTG | GAGATACATCTACAACC | GACACAACTACGAGCTG |
| GGG (SEQ ID NO: 1453) | GGC (SEQ ID NO: 1462) | GGG (SEQ ID NO: 1453) | GGC (SEQ ID NO: 1462) |
| TGGAGAGATACATCTAC | GTGCAGACACAACTACG | TGGAGAGATACATCTAC | GTGCAGACACAACTACG |
| AAC (SEQ ID NO: 1454) | AGC (SEQ ID NO: 1463) | AAC (SEQ ID NO: 1454) | AGC (SEQ ID NO: 1463) |
| GGTTGTAGATGTATCTCT | GAGATACATCTACAACC | GGTTGTAGATGTATCTC | GAGATACATCTACAACC |
| CC (SEQ ID NO: 1455) | GGG (SEQ ID NO: 1453) | TCC (SEQ ID NO: 1455) | GGG (SEQ ID NO: 1453) |
| GGAGAGATACATCTACA | AGACACAACTACGAGCT | GGAGAGATACATCTACA | AGACACAACTACGAGCT |
| ACC (SEQ ID NO: 1456) | GGG (SEQ ID NO: 1464) | ACC (SEQ ID NO: 1456) | GGG (SEQ ID NO: 1464) |
| AGGAATGCTACGCGTTT | TGGAGAGATACATCTAC | TTACGTGTACCAGTTAC | TGGAGAGATACATCTAC |
| AAT (SEQ ID NO: 1457) | AAC (SEQ ID NO: 1454) | GGC (SEQ ID NO: 1466) | AAC (SEQ ID NO: 1454) |
| CAGGAATGCTACGCGTT | GGTTGTAGATGTATCTCT | AGGAATGCTACGCGTTT | GGTTGTAGATGTATCTCT |
| TAA (SEQ ID NO: 1458) | CC (SEQ ID NO: 1455) | AAT (SEQ ID NO: 1457) | CC (SEQ ID NO: 1455) |
| TTACGTGTACCAGGGAC | GGAGAGATACATCTACA | CGAAGCGCACGAACTCC | GGAGAGATACATCTACA |
| GGC (SEQ ID NO: 1459) | ACC (SEQ ID NO: 1456) | TCC (SEQ ID NO: 1359) | ACC (SEQ ID NO: 1456) |
| TAATGGGACACAGCGCT | AGGAATGCTACGCGTTT | AGAATTACGTGTACCAG | AGGAATGCTACGCGTTT |
| TCC (SEQ ID NO: 1460) | AAT (SEQ ID NO: 1457) | TTA (SEQ ID NO: 1467) | AAT (SEQ ID NO: 1457) |
| CGCGCGCTTCGACAGCG | TGCAGACACAACTACGA | CAGGAATGCTACGCGTT | TGCAGACACAACTACGA |
| ACG (SEQ ID NO: 1217) | GCT (SEQ ID NO: 1465) | TAA (SEQ ID NO: 1458) | GCT (SEQ ID NO: 1465) |
| ATGCAGACACAACTACG | TAATGGGACACAGCGCT | TAATGGGACACAGCGCT | CAGGAATGCTACGCGTT |
| AGC (SEQ ID NO: 1461) | TCC (SEQ ID NO: 1460) | TCC (SEQ ID NO: 1460) | TAA (SEQ ID NO: 1458) |

TABLE 17
HLA-DPB1 04:02, 11:01, 17:01, and 34:01

| DPB1*04:02 | DPB1*11:01 | DPB1*17:01 | DPB1*34:01 |
|---|---|---|---|
| GACACAACTACGAGCTG | GTGCAGACACAACTACG | GTGCAGACACAACTACG | GTGCAGACACAACTACG |
| GGC (SEQ ID NO: 1462) | AGC (SEQ ID NO: 1463) | AGC (SEQ ID NO: 1463) | AGC (SEQ ID NO: 1463) |
| GTGCAGACACAACTACG | TGGAGAGATACATCTAC | GAGATACATCTACAACC | GAGATACATCTACAACC |
| AGC (SEQ ID NO: 1463) | AAC (SEQ ID NO: 1454) | GGG (SEQ ID NO: 1453) | GGG (SEQ ID NO: 1453) |
| GAGATACATCTACAACC | GGTTGTAGATGTATCTCT | TGGAGAGATACATCTAC | TGGAGAGATACATCTAC |
| GGG (SEQ ID NO: 1453) | CC (SEQ ID NO: 1455) | AAC (SEQ ID NO: 1454) | AAC (SEQ ID NO: 1454) |
| AGACACAACTACGAGCT | TTACGTGTACCAGTTAC | GGTTGTAGATGTATCTCT | GGTTGTAGATGTATCTCT |
| GGG (SEQ ID NO: 1464) | GGC (SEQ ID NO: 1466) | CC (SEQ ID NO: 1455) | CC (SEQ ID NO: 1455) |
| TGGAGAGATACATCTAC | AGGAATGCTACGCGTTT | GGAGAGATACATCTACA | GGAGAGATACATCTACA |
| AAC (SEQ ID NO: 1454) | AAT (SEQ ID NO: 1457) | ACC (SEQ ID NO: 1456) | ACC (SEQ ID NO: 1456) |
| GGTTGTAGATGTATCTCT | GAGATACATCTACAACC | AGGAATGCTACGCGTTT | AGGAATGCTACGCGTTT |
| CC (SEQ ID NO: 1455) | GGC (SEQ ID NO: 1468) | AAT (SEQ ID NO: 1457) | AAT (SEQ ID NO: 1457) |
| GGAGAGATACATCTACA | AGAATTACGTGTACCAG | TTACGTGCACCAGTTAC | GACACAACTACGAGCTG |
| ACC (SEQ ID NO: 1456) | TTA (SEQ ID NO: 1467) | GGC (SEQ ID NO: 1470) | GTC (SEQ ID NO: 1472) |
| AGGAATGCTACGCGTTT | CAGGAATGCTACGCGTT | AGAATTACGTGCACCAG | TAATGGGACACAGCGCT |
| AAT (SEQ ID NO: 1457) | TAA (SEQ ID NO: 1458) | TTA (SEQ ID NO: 1471) | TCC (SEQ ID NO: 1460) |
| TGCAGACACAACTACGA | TAATGGGACACAGCGCT | CGAAGCGCACGAACTCC | TTACCTTTTCCAGGGACG |
| GCT (SEQ ID NO: 1465) | TCC (SEQ ID NO: 1460) | TCC (SEQ ID NO: 1359) | GC (SEQ ID NO: 1473) |
| CGAAGCGCACGAACTCC | CGTAGCATTCCTGCCGT | CAGGAATGCTACGCGTT | CAGGAATGCTACGCGTT |
| TCC (SEQ ID NO: 1359) | AAC (SEQ ID NO: 1469) | TAA (SEQ ID NO: 1458) | TAA (SEQ ID NO: 1458) |

### Example 6: Evaluating Off-Target Editing in HLA Knockout HSCs

The HLA genes are located on the short arm of chromosome 6 (e.g., as shown in FIG. 11), and because the HLA system contains closely related genes, specific gRNA design is challenging. Sequencing of the triple knockout (HLA edited) clones was performed to check for unwanted editing and to ensure that no major editing events, e.g., deletion(s), occurred within other regions of chromosome 6. Sequencing methods and other analyses were performed to evaluate the degree of gRNA off-target activity and to select gRNAs that represent a low risk of affecting non-target HLA genes.

Sequencing was performed by using in situ break labelling in fixed and permeabilized cells by ligating a full-length P5 sequencing adapter to end-prepared DSBs. Genomic DNA was extracted, fragmented, end-prepared, and ligated using a chemically modified half-functional P7 adapter. The resulting DNA libraries contained a mixture of functional DSB-labelled fragments (P5:P7) and non-functional genomic DNA fragments (P7:P7). Subsequent DNA sequencing of the DNA libraries enriched for DNA-labelled fragments, eliminating all extraneous non-functional DNA. As the library preparation is PCR-free, each sequencing read obtained was equivalent to a single labelled DSB-end from a cell. This generated a DNA break readout, enabling the direct detection and quantification of genomic DSBs by sequencing without the need for error-correction and enabled mapping a clear list of off-target mutations.

Table 18 below summarizes the results of the editing strategy in two representative HLA edited clones relative to wild-type (gHSCs) cells.

TABLE 18
Clonal HSC HLA knockouts.

| Sample ID | Locus | Allele 1 | Allele 2 | Comments |
|---|---|---|---|---|
| gHSCs | A | A*01:01:01 | A*01:01:01 | Not affected |
| | B | B*08:01:01 | B*08:01:01 | Not affected |
| | C | C*07:01:01 | C*07:01:01 | Not affected |
| | DPA1 | DPA1*01:03:01 | DPA1*02:01:02 | Not affected |
| | DPB1 | DPB1*01:01:01 | DPB1*04:01:01 | Not affected |
| | DQA1 | DQA1*05:01:01 | DQA1*05:01:01 | Not affected |
| | DQB1 | DQB1*02:01:01 | DQB1*02:01:01 | Not affected |
| | DRB1 | DRB1*03:01:01 | DRB1*03:01:01 | Not affected |
| | DRB345 | DRB3*01:01:02 | DRB3*01:01:02 | Not affected |
| Clone #1 | A | xxx | xxx | Deletion in Exon 2 |
| | B | B*08:01:01 | B*08:01:01 | Not affected |
| | C | C*07:01:01 | C*07:01:01 | Not affected |
| | DPA1 | DPA1*01:03:01 | DPA1*02:01:02 | Not affected |
| | DPB1 | xxx | xxx | Deletion in Exon 2 |
| | DQA1 | DQA1*05:01:01 | DQA1*05:01:01 | Not affected |
| | DQB1 | xxx | xxx | Deletion in Exon 2 |
| | DRB1 | DRB1*03:01:01 | DRB1*03:01:01 | Not affected |
| | DRB345 | DRB3*01:01:02 | DRB3*01:01:02 | Not affected |
| Clone #2 | A | xxx | xxx | Deletion in Exon 2 |
| | B | B*08:01:01 | B*08:01:01 | Not affected |
| | C | C*07:01:01 | C*07:01:01 | Not affected |
| | DPA1 | DPA1*01:03:01 | DPA1*02:01:02 | Not affected |
| | DPB1 | xxx | xxx | Deletion in Exon 2 |
| | DQA1 | DQA1*05:01:01 | DQA1*05:01:01 | Not affected |
| | DQB1 | xxx | xxx | Deletion in Exon 2 |
| | DRB1 | DRB1*03:01:01 | DRB1*03:01:01 | Not affected |
| | DRB345 | DRB3*01:01:02 | DRB3*01:01:02 | Not affected |

Table 19 provides a non-limiting example of gRNAs used in the experiments. The gRNA sequences disclosed in Tables 2A through 17 can be used to knock out expression of indicated HILA genes.

TABLE 19
Exemplary gRNA sequences

| gRNA | |
|---|---|
| ID | Spacer sequence |
| HLA-A | CGTAGCCCACGGCGATGAAG (SEQ ID NO: 56) |
| | ATTTCTTCACATCCGTGTCC (SEQ ID NO: 7) |
| | GTCTCCTGGTCCCAATACTC (SEQ ID NO: 91) |
| | GTAGCCCACGGCGATGAAGC (SEQ ID NO: 54) |
| | TAGCCCACGGCGATGAAGCG (SEQ ID NO: 52) |
| | GAGGGTTCGGGGCGCCATGA (SEQ ID NO: 1) |
| | CCGGAACACACGGAATGTGA (SEQ ID NO: 157) |
| | ACAGCGACGCCGCGAGCCAG (SEQ ID NO: 59) |
| | CCACTCACAGATTGACCGAG (SEQ ID NO: 202) |
| | CGACGCCGCGAGCCAGAGGA (SEQ ID NO: 61) |
| | CAGATTGACCGAGTGGACCT (SEQ ID NO: 206) |
| | CAGACTGACCGAGTGGACCT (SEQ ID NO: 132) |
| | ACAGACTGACCGAGTGGACC (SEQ ID NO: 131) |
| | GCCGGGCCGGGACACGGATG (SEQ ID NO: 141) |
| | CCAGGAGACACGGAATGTGA (SEQ ID NO: 126) |
| | CCAGTCACAGACTGACCGAG (SEQ ID NO: 129) |
| | TCGACAGCGACGCCGCGAGC (SEQ ID NO: 120) |
| HLA- | GAGATACATCTACAACCGGG (SEQ ID NO: 1453) |
| DPB1 | TGGAGAGATACATCTACAAC (SEQ ID NO: 1454) |
| | GGTTGTAGATGTATCTCTCC (SEQ ID NO: 1455) |
| | GGAGAGATACATCTACAACC (SEQ ID NO: 1456) |
| | AGGAATGCTACGCGTTTAAT (SEQ ID NO: 1457) |
| HLA- | CAGATACATCTATAACCGAG (SEQ ID NO: 1094) |
| DQB1 | CGAGTACTGGAACAGCCAGA (SEQ ID NO: 1067) |
| | CCCGTTGGTGAAGTAGCACA (SEQ ID NO: 1090) |
| | GTGCTACTTCACCAACGGGA (SEQ ID NO: 1091) |
| | AGGTCGTGCGGAGCTCCAAC (SEQ ID NO: 1080) |
| | AGGTGACAGTTGCTACCCGA (SEQ ID NO: 1153) |
| | TCCCGTTGGTGAAGTAGCAC (SEQ ID NO: 1164) |
| | AACTACGAGGTGGCGTACCG (SEQ ID NO: 1197) |
| | CTGTTCCAGTACTCGGCAAC (SEQ ID NO: 1181) |
| | ACTACGAGGTGGCGTACCGC (SEQ ID NO: 1198) |
| | CGAAGCGCACGTACTCCTCT (SEQ ID NO: 1171) |

The results show that the editing strategy was successful in selectively targeting the HLA-A, DPB1, and DQB1 genes without affecting the other HLA genes or introducing major deletion elsewhere.

These results were confirmed by a phenotypic analysis of the HLA edited clones by FACS and immunofluorescence. As shown in FIGS. 12A and B, HLA edited cells tested positive for overall expression of HLA class-I molecules, comparable to the overall expression of HLA class-I molecules of wild-type cells. Specific expression of HLA-A via immunofluorescence confirmed that HLA-A was not expressed in the HLA edited cells, corroborating the finding that the gene editing strategy was successful in deleting only the HLA-A gene. Specifically, FIG. 12A, shows that the HLA edited cells were all positive for class-I like HLA to the same extent as the wild type (gHSC) cells. This result indicates that despite the deletion of HLA-A, other class-I molecules like HLA-B and C were expressed and not affected by the gene editing strategy.

To confirm that the HLA-A gene was deleted, specific expression of the HLA-A was analyzed with immunofluorescence. As can be seen in FIG. 12B, HLA-A was not expressed in the HLA edited clone indicating that the gene editing strategy was efficient in specifically deleting the HLA-A gene only. Such preservation of overall class-I expression with deletion of HLA-A will facilitate patient matching while avoiding NK-cell mediated rejection.

### Example 7: Evaluating Pluripotency and Immunecompatability of HLA Edited HSCs

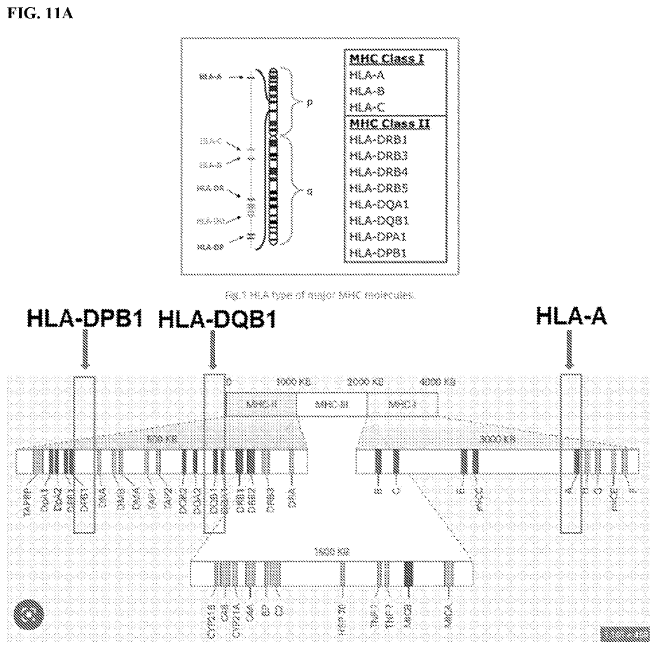

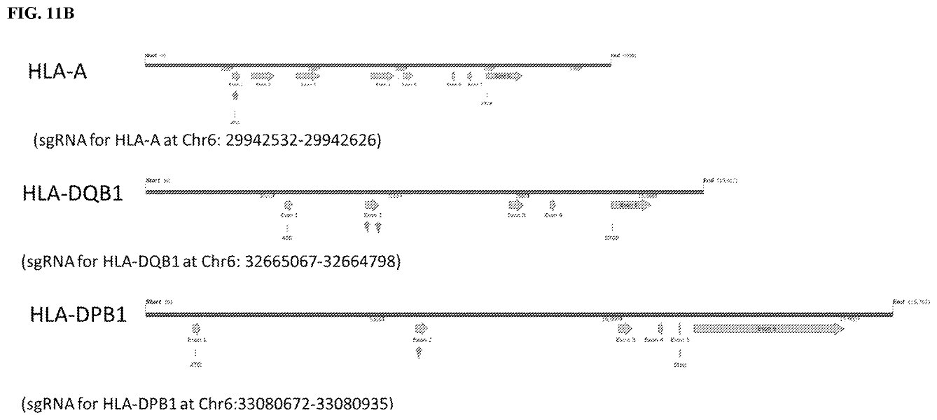

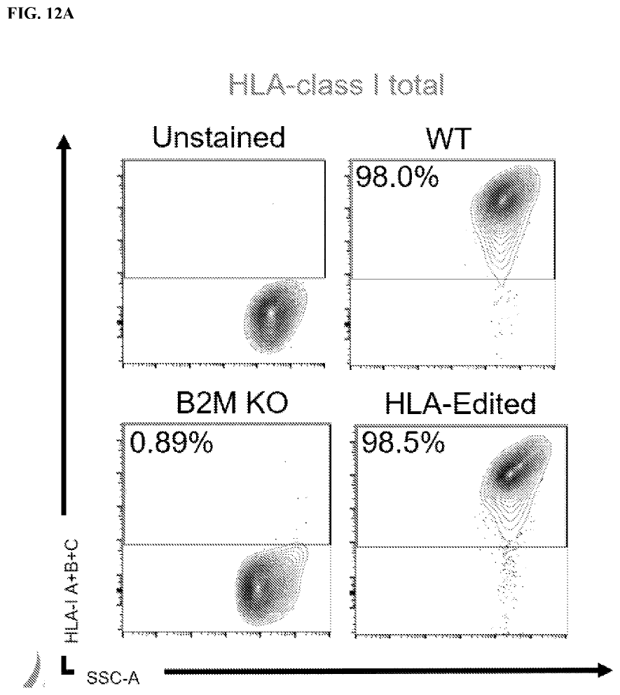

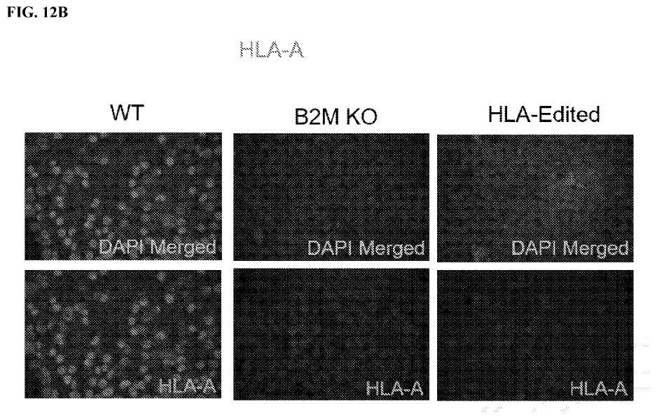

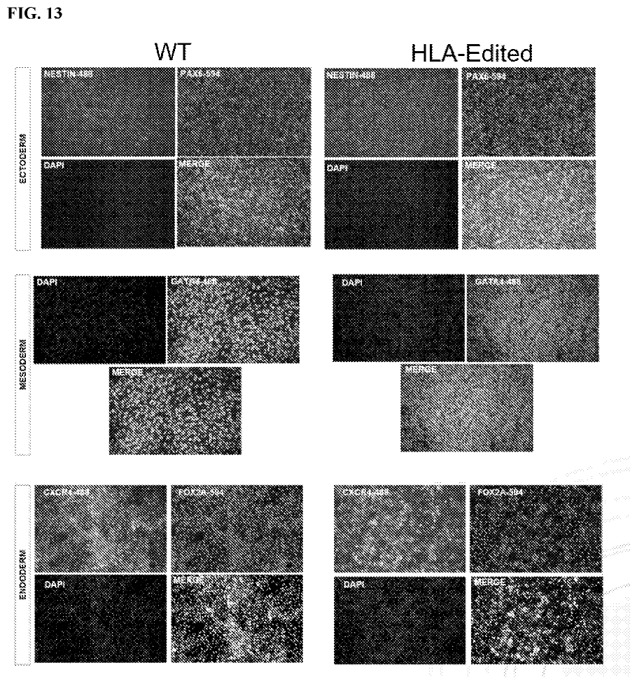

The ability of HLA edited cells to preserve pluripotency was evaluated. As shown in FIG. 13, immunofluorescence evaluation of the HLA edited iPSC clones indicated that they maintained trilineage differentiation, with ectoderm differentiation indicated by NESTIN-488 and PAX6-594 staining, mesoderm differentiation indicated by GATA-488 staining, and endoderm differentiation indicated by CXCR4-488 and FOX2A-594 staining. HLA class I molecules are expressed on the surface of all nucleated cells and if the HLA class I molecules are mismatched between donor and recipient, then the cells could be recognized and killed by CD8+ T cells. Additionally, HLA mismatching could lead to cytokine release syndrome (CRS) and graft-versus-host disease (GVHD). Conversely, the complete deletion of HLA-I molecules, via B2M KO, would make the cell a target of NK cell-mediated cytotoxicity. The preservation of overall class-I expression with deletion of HLA-A can facilitate patient matching while preventing the NK-cell mediated rejection. Thus, the immunocompatibility of the HLA edited HSCs was tested by co-culture with peripheral blood mononuclear cells (PBMCs) to evaluate if the immune cells would reject a graft of the HLA edited and gHSCs.

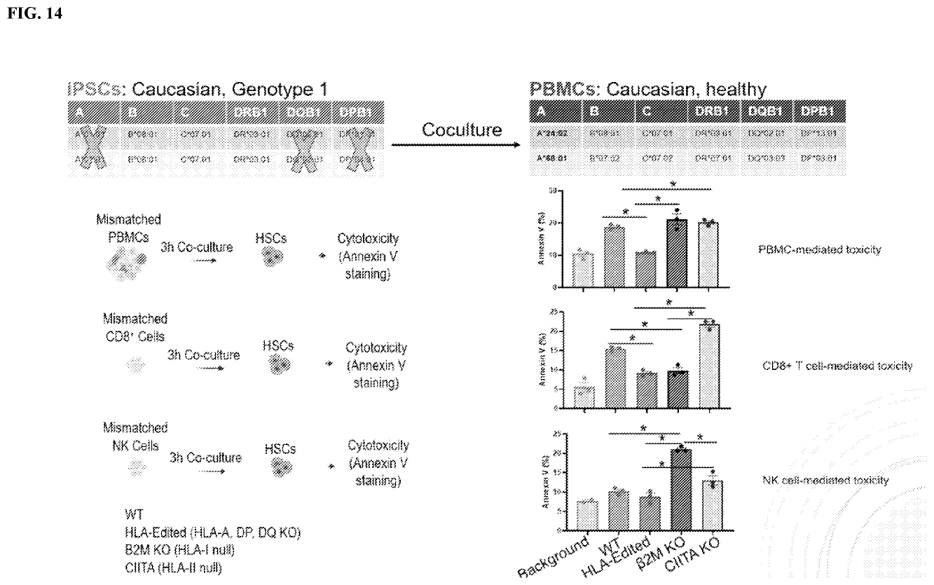

Wild-type (gHSCs) and HLA edited HSCs were co-cultured with PBMCs matching the HLA-B and HLA-C markers, but with mismatched HLA-A. B2M KO HSCs lacking expression of HLA class-I molecules and CIITA KO HSCs lacking expression of class-II molecules were used as controls to compare the degree of PBMC-mediated cytotoxicity for HLA-null and mismatched HLA, respectively. FIG. 14 shows the results of the PBMC-mediated cytotoxicity assay in the co-cultures as measured by an annexin V staining. The results show that deletion of HLA-A in the HLA edited HSCs protects the cells from PBMC-mediated cytotoxicity, while WT, B2M KO, and CIITA KO were susceptible to PBMC-mediated cytotoxicity. Co-cultured HSCs with sorted CD8+ T cells from the same PBMC donor protected HLA edited and B2M KO HSCs from CD8+ T cell cytotoxicity. Conversely, co-cultured HSCs with sorted NK cells only protected the WT (gHSCs) and HLA edited cells from the NK cell-mediated cytotoxicity.

In summary, the immune capability results show that the CD8+ T cells present in the PBMC samples were responsible for killing the cells with mismatched HLA molecules (WT) and CIITA KO, while the NK cells present in the PBMCs were responsible for killing the HLA-null cells (B2M KO). However, HLA edited HSCs are protected from CD8+ T cell-mediated cytotoxicity (because the mismatched HLA-A had been knocked out), and protected from NK cell-mediated cytotoxicity (because HLA class I molecule expression was largely preserved).

### Example 8: Evaluating the In Vivo Engraftment Potential of HLA Edited HSC's

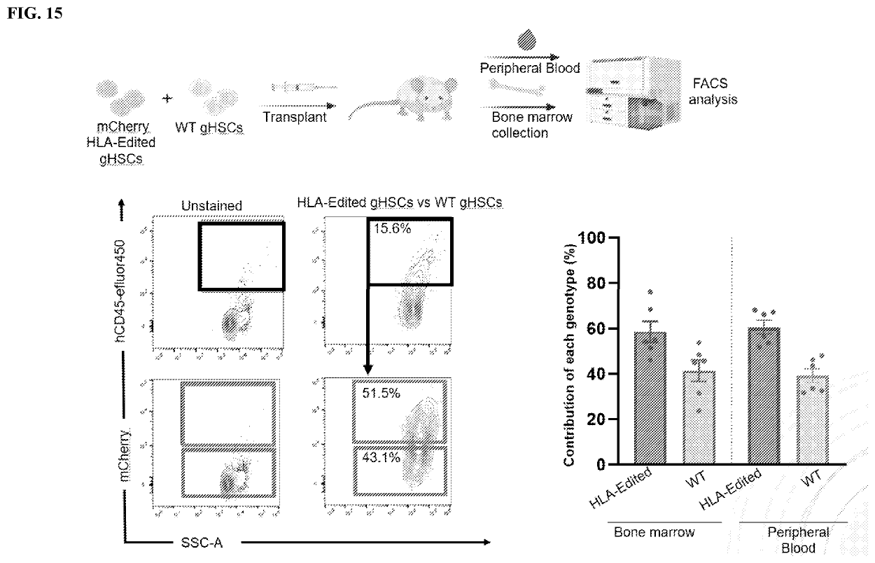

To evaluate the engrafting potential of HLA edited HSCs, the cells' ability to engraft in vivo was evaluated by a competitive transplant against WT HSCs. Equal proportions of mCherry HLA edited HSCs and unedited HSCs were admixed and transplanted into mice, from where bone marrow (BM) and peripheral blood samples were recovered and evaluated by FACS to compare the relative amounts of each cell type present in the samples. As shown in FIG. 15, both the HLA edited HSCs and the WT HSCs contributed to approximately equal engraftment in the BM and peripheral blood samples. These results confirm that HLA edited HSCs (prepared according to this disclosure) are comparable to WT HSCs in their engraftment and reconstitution potential. Hence, it is expected that properties of the WT HSCs are consistent with that of the HLA edited HSCs of the present disclosure.

### Example 9: Differentiation of HLA Edited HSCs to Hematopoietic Lineages

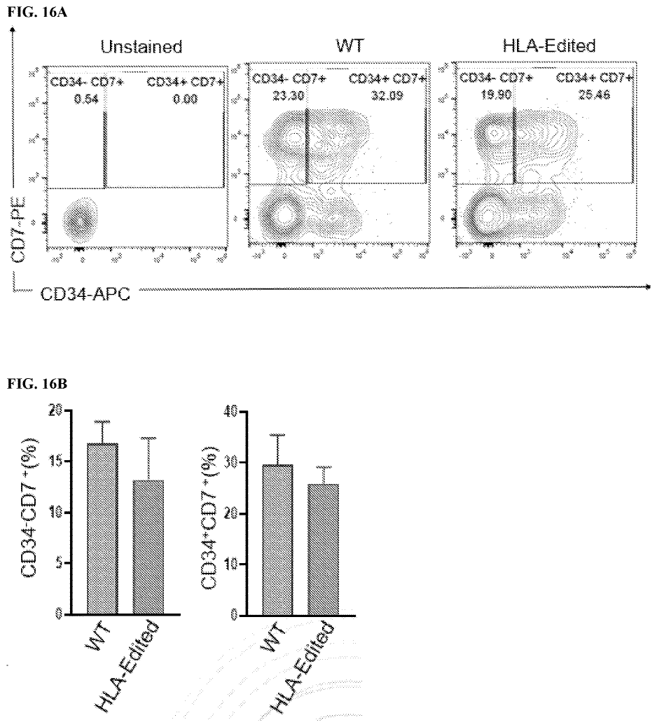

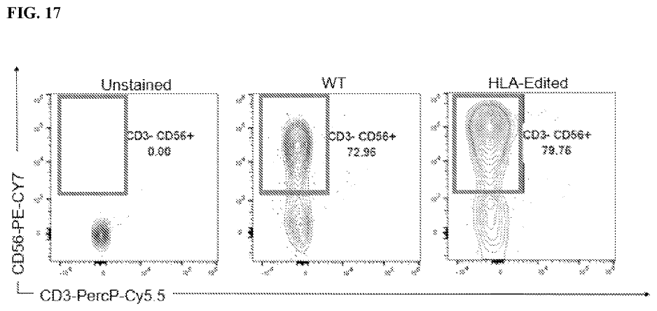

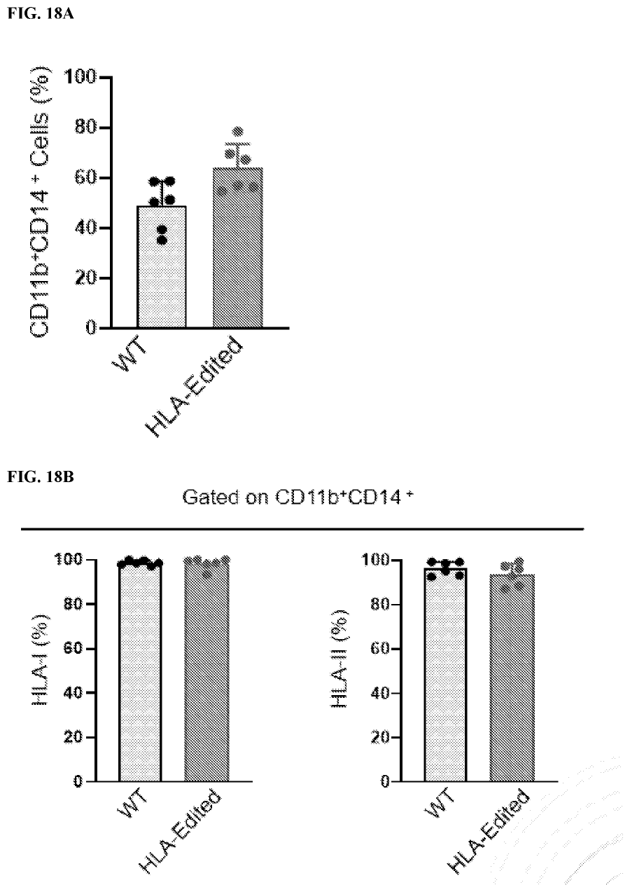

Experiments were carried out to determine if HLA deletion impacts the HSCs ability to differentiate into different types of immune cells. Using the process essentially as described in Example 2, HLA edited HSCs were differentiated to pro-T cells. It was found that the HLA-edited HSCs were able to differentiate into Pro-T Cells, which was comparable to WT (non-HLA-edited) HSCs as measured by their CD34+-CD7+ expressions (FIGS. 16A and B). Further, upon differentiation to NK cells, it was shown that HLA-edited HSCs were able to differentiate into NK cells, comparable to WT HSCs as measured by their CD3-CD56+ expressions (FIG. 17). Likewise, it was determined that the HLA-edited HSCs were able to differentiate into monocyte/macrophage lineage comparable to WT HSCs as measured by their CD11b+−CD14+ expressions (FIG. 18A). Further, the CD11b+-CD14+ gated population showed equivalent HLA-I and HLA-II expression (FIG. 18B) indicating that HLA-edited HSCs preserve the overall expression of both class I and class II molecules.

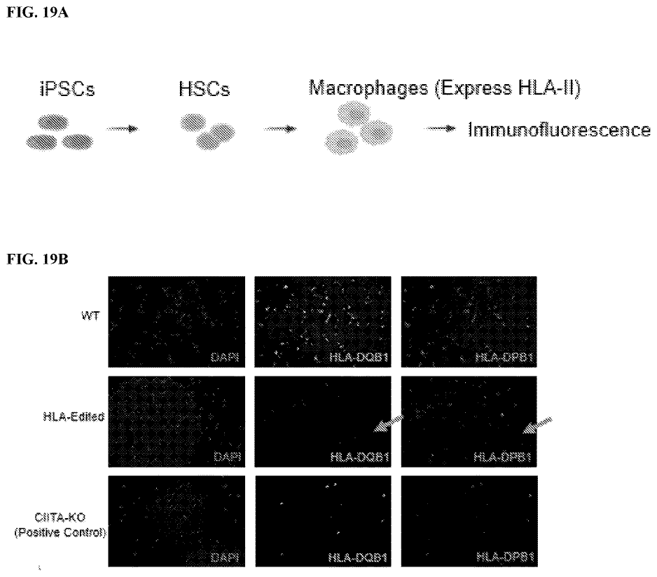

The overall expression of the other class-II molecules in HLA-DQB1 and HLA-DPB1 supported by the edited HSCs was evaluated, by evaluating the expression in macrophages differentiated from the HSCs. The design of the study is schematically shown in FIG. 19A. It was found that the deletion of HLA-DQB1 and HLA-DPB1 did not affect the expression of other HLA Class II molecules (FIG. 19B). For example, HLA-DR is comparably expressed in both WT and HLA-edited cells (FIG. 19C). In FIGS. 19B and C, CIITA-KO is as a positive control.

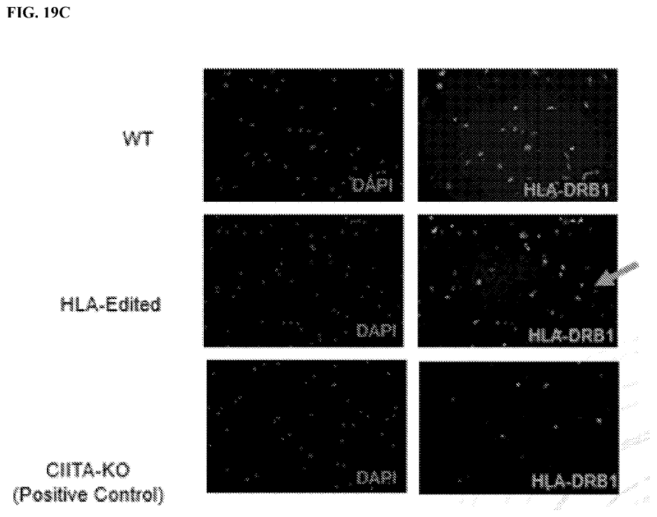

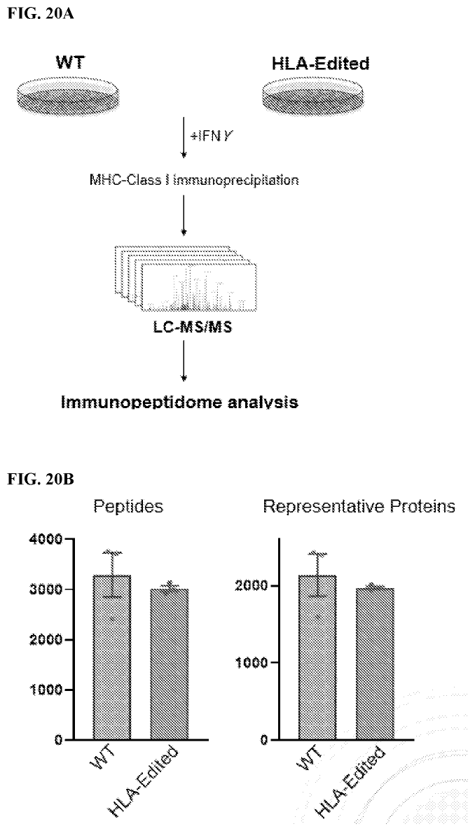

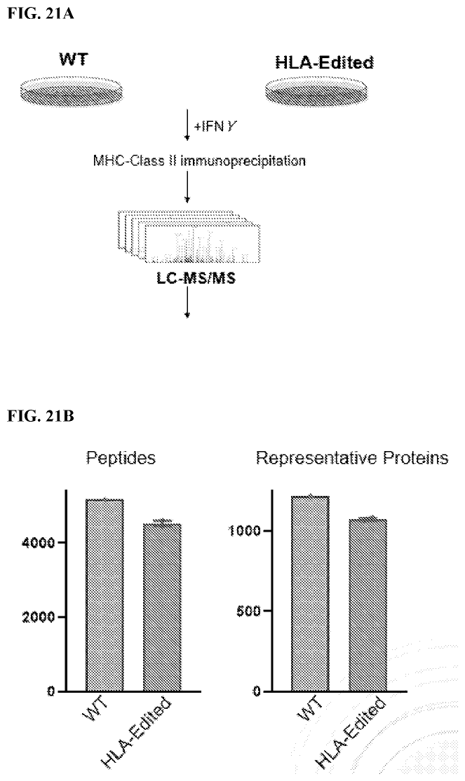

Antigen presenting cells (APC) present antigens to helper CD4+ T cells through the HLA-II molecules. Activation of helper CD4 ‘T cells promotes the generation of antigen-specific CD8″ T cells which further develop into antigen-specific CTLs. Likewise, HLA Class I molecules are expressed on the surface of all nucleated cells and display peptide fragments of proteins from within the cell to CD8+ CTLs. CTLs induce cytotoxic killing of target (infected) cells upon recognition of HLA-I-peptide complex expressed on the cell surface. Hence, a study was carried out to determine if deletion of HLA-A impacts the edited HSCs’ class I peptide presentation. As shown in FIGS. 20A and B, immunopeptidome analysis shows that the deletion of HLA-A does not impact overall class I peptide presentation. HLA-A edited cells showed comparable peptide and protein presentation when compared to wild type (gHSCs) HSCs. Further, as shown in FIGS. 21A and B, deletion of HLA-DQB1 and HLA-DPB1 does not impact overall class II peptide presentation by macrophages differentiated from the HSCs. Together, these data suggest that despite the deletion of HLA-A, HLA-DQ, and HLA-DP molecules, the cells preserve their ability to present a broad spectrum of class I and II peptides.

### Example 10: In Vivo Testing of Antigen-Mediated Immune Response

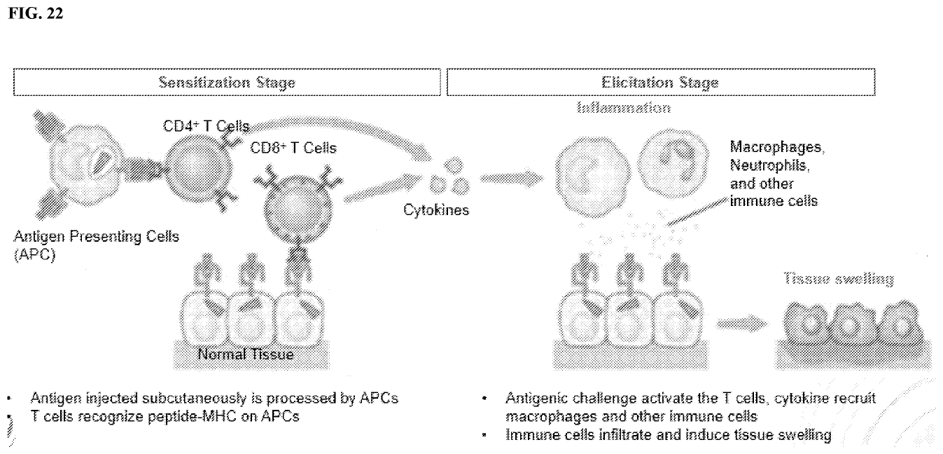

FIG. 22 is a schematic illustration of a Delayed Type Hypersensitivity Reaction, showing the sensitizing and eliciting stages of an antigen presentation. Briefly, upon antigen injection, antigen is processed by antigen presenting cells (APC) and presented by MHC Class II molecules on the APC surface. CD4+ T cells recognize peptide-MHC on antigen presenting cells (APCs). Upon antigenic challenge CD4+ helper T cells are activated and cytokines recruit macrophages and other immune cells, which induce tissue swelling at the site of antigen exposure.

A delayed-type hypersensitivity assay was performed on transplanted mice. Specifically, the mice were sensitized by subcutaneous injection of sheep Red blood cells as antigen. If the mice have a functional immune system, the APCs process the antigen and present peptide antigens to CD4+ T cells. Next, the mice were challenged by subcutaneous injection of the same antigen in the left paw. At this point the T cells are activated and secrete cytokines which recruit macrophages and other immune cells at the site of antigen injection creating tissue swelling. In this assay, a functional immune system could result in the swelling of the left paw as measured with a micro caliper.

As can be seen in FIGS. 23A and B, the control (non-transplanted) mice did not show any left paw swelling as they are immunodeficient. Conversely, the mice transplanted with Cord Blood CD34+ cells showed tissue swelling and doubled the diameter of their left paw. A similar immune system response was found in both the mice transplanted with the WT (non-edited HSCs) and the HLA-edited HSCs (Triple KO).

### Example 11: In Vivo Testing of HSC-Derived T Cells

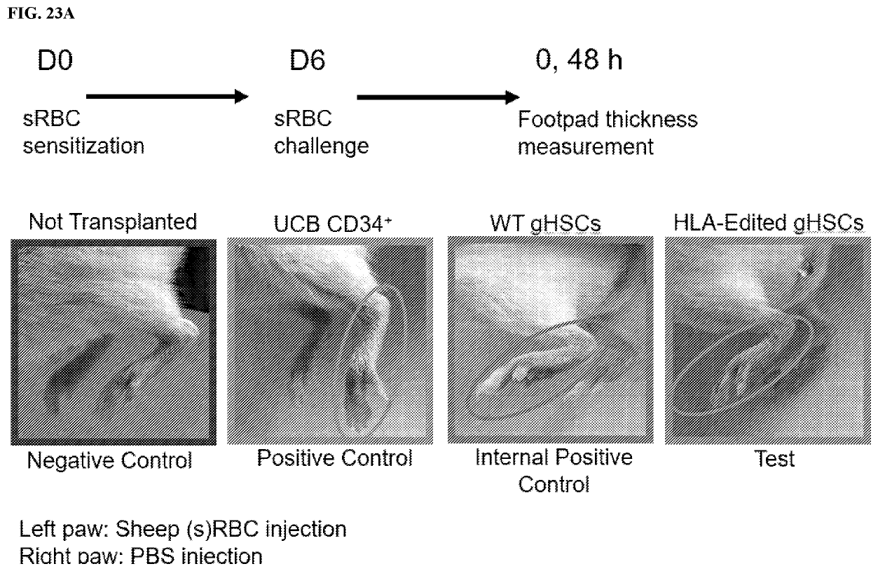

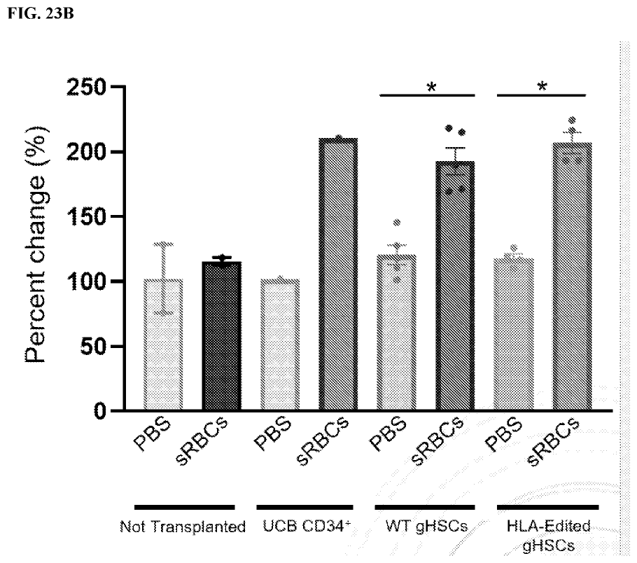

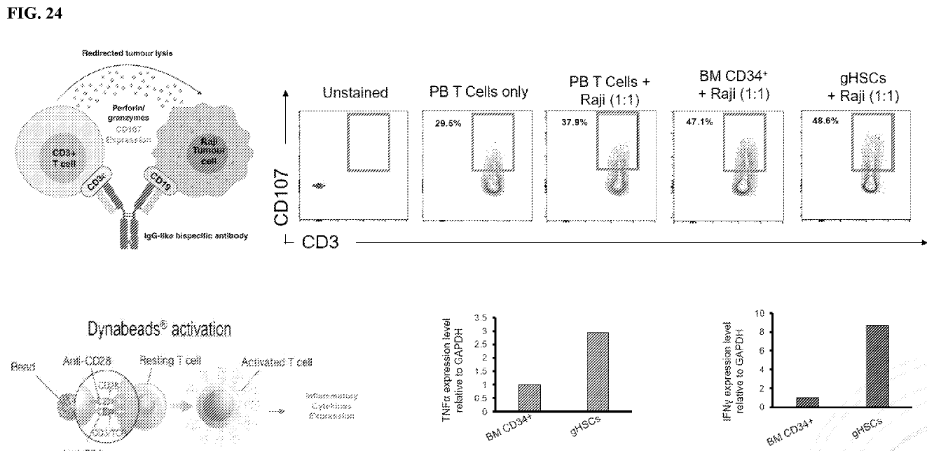

In-vitro activation of the HSC-derived T cells was measured, and results are illustrated in FIG. 24. Top panel of FIG. 24 shows FACS analysis of activated T cells from different sources, including the HSCs prepared using Piezol activation. T cells prepared from the HSCs demonstrated comparable or superior activation as measured by increased CD107 expression. The lower panel shows Dynabeads activation, where activated T cells express inflammatory cytokines. HSC-derived T cells prepared using Piezol activation expressed higher levels of inflammatory cytokines as exemplified by TNF-alpha and interferon gamma expression levels.

### Example 12: Evaluating Properties of CCR5 Knock Out HSCs to Develop into Pro-T Cells

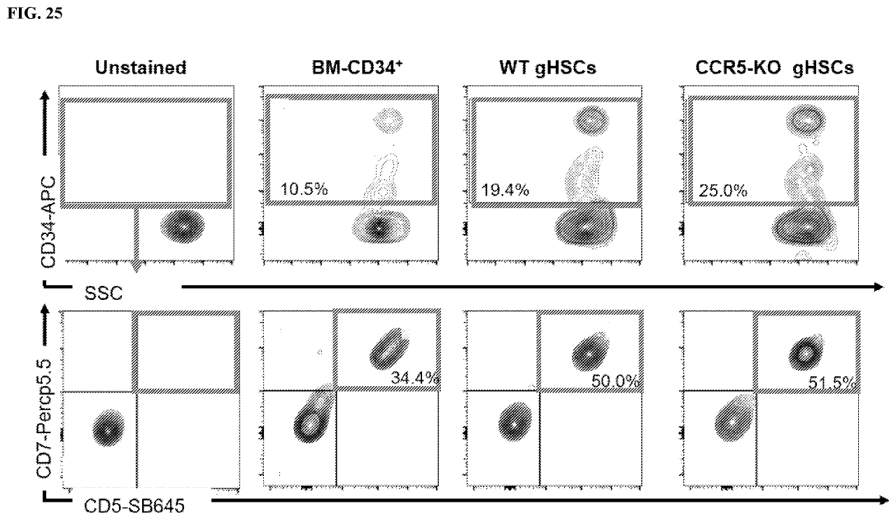

To determine if CCR5 knock out (CCR5-KO) HSCs can comparably differentiate to pro-T cells similar to their wild-type counterparts from which they are derived, a study was performed in which the CD34, CD7 and CD5 expression of the HSCs and the CCR5-KO were measured. As can be seen in FIG. 25, HSCs successfully differentiated into CD34+CD7+CD5+ pro-T cells comparably to bone marrow derived CD34+ cells. Likewise, the CCR5-KO, like their gHSC counterpart, successfully differentiated into CD34+CD7+CD5+ pro-T cells.

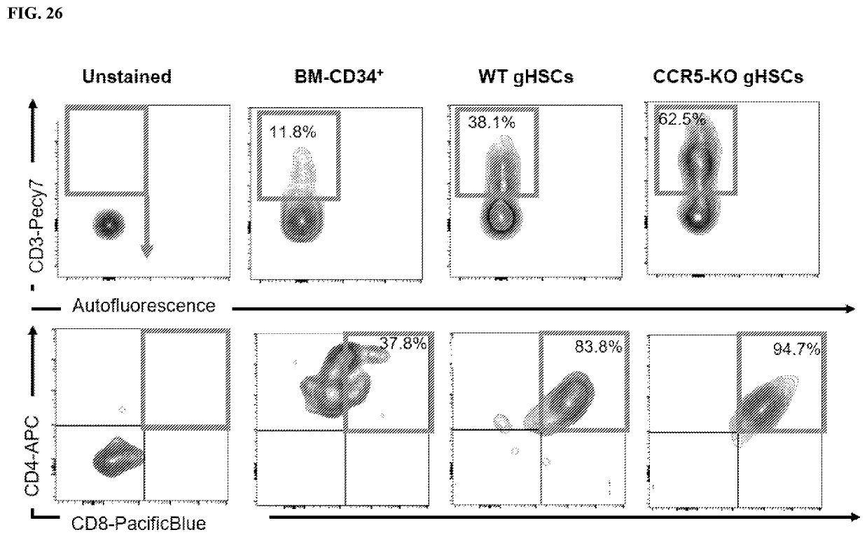

Next, the property of CCR5-knocked out HSCs to differentiate into double positive (CD4+CD8+) T cells was assessed. As can be seen in FIG. 26, CCR5-knocked out HSCs comparably differentiated into double positive (CD4+CD8+) T cells when compared to their gHSC counterparts from which they were derived (i.e., HSCs of the present disclosure).

### Example 13—Evaluating HSC-Derived T Cell (Pro-T Cell) Differentiation and Maturation

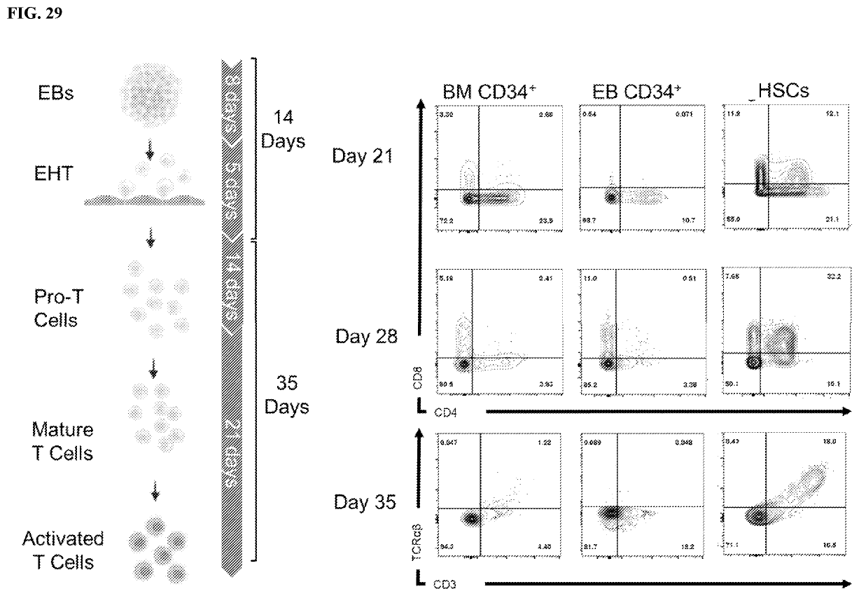

Next, the ability of the HSC-derived T cells (pro-T cells) to differentiate into mature T cells was tested. After a 35-day differentiation period, pro-T cells were evaluated by cell sorting for the presence of CD4+, CD8+, and AB+ T cell populations. As shown in FIG. 29, pro-T cells differentiated into CD4+, CD8+, and αβ+ T cells more efficiently than bone marrow (BM)-derived CD34+ cells and the CD34+ cells derived from the embryonic bodies (EB).

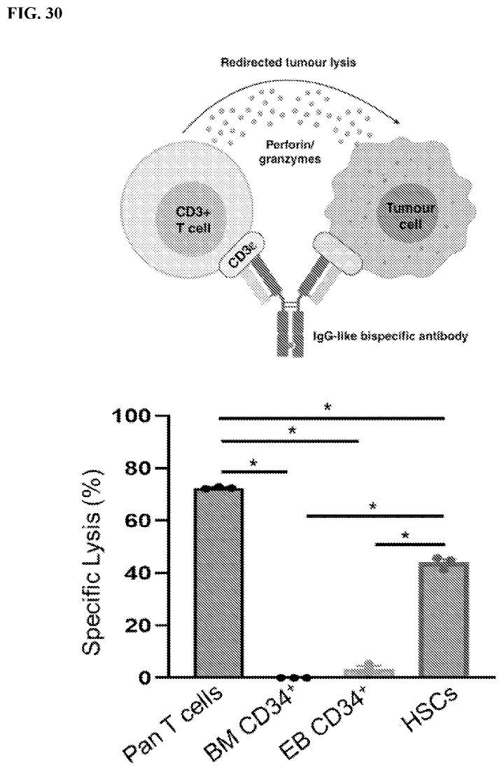

Next, to test the functional properties, each of the T cell populations were co-cultured with a CD19+ lymphoma cell line and an anti-CD3/CD-19 bispecific antibody. In this experimental model, the bispecific antibody engaged both the CD3 receptor on T cells and the CD19 cell surface receptor of the lymphoma cells, thus triggering T cell activation. The degree of activation was evaluated by measuring the subsequent T-cell mediated cytotoxicity in comparison to a Pan T cell control. As shown in FIG. 30, the pro-T cells exhibited a statistically significant outperformance in cytotoxicity in comparison to both the BM CD34+ T cells and the EB CD34+ T cells.

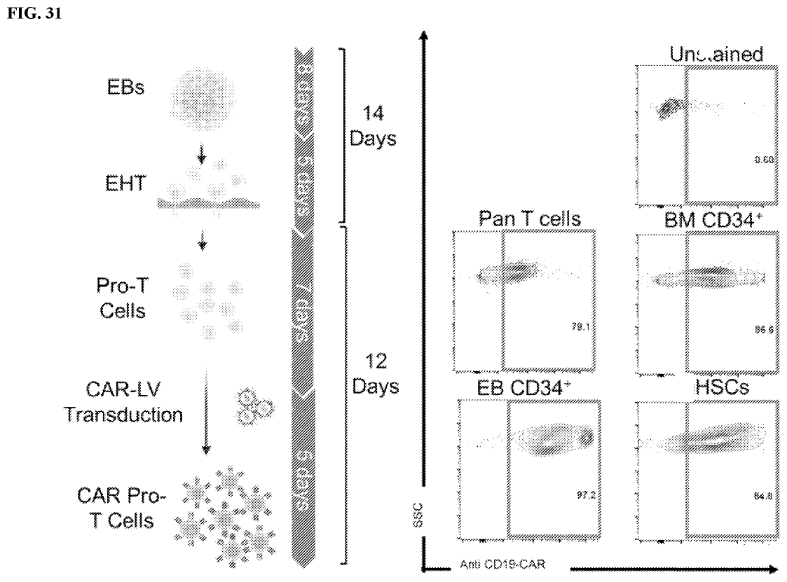

Because the overall differentiation process of pro-T cells is 35 days long, a transduction experiment was performed to test if the time required to differentiate the HSCs could be decreased. Pro-T cells were cultured in an activation media (for approx. 7 days) to increase the transduction efficiency of the cells. Next, the cells were transduced with lentiviral (LV) particles encoding an anti-CD19 CAR transgene. The cells were cultured for additional 4-5 days (a total of 12 days) and their maturation and killing capabilities were evaluated. As shown in FIG. 31, the HSC-derived pro-T cells can be transduced with high efficiency, with more than 80% of the cells express the anti-CD19 CAR as evidenced by cell sorting.

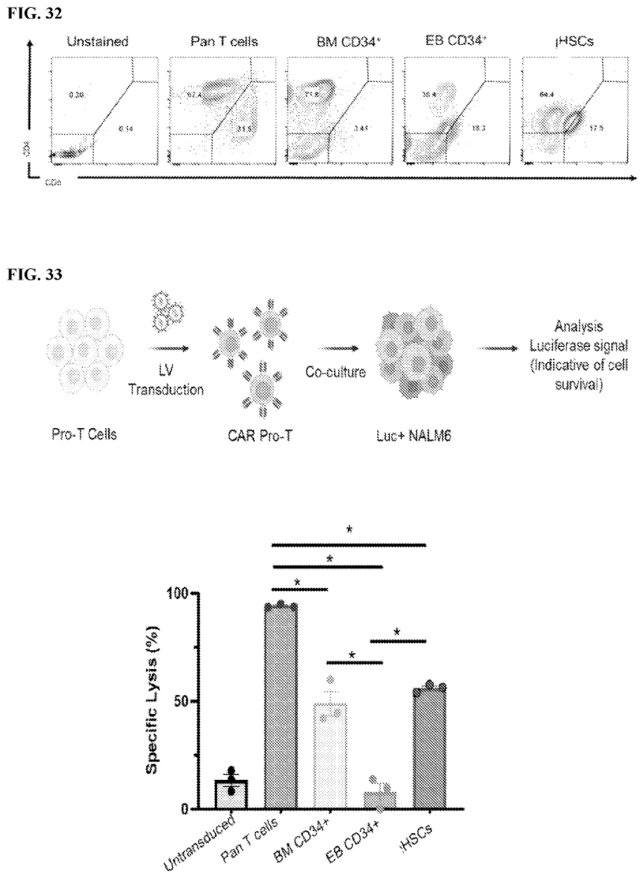

Next, the pro-T cells were evaluated for their ability to effectively mature into CD4+/CD8+ T cells via CAR transduction. The pro-T cells, along with bone marrow (BM)-derived CD34+ cells and CD34+ cells derived from the embryonic bodies (EB) (and Pan T cells as a positive control), underwent LV-transduction with the anti-CD19 CAR. The T cell subsets were screened by cell sorting for the presence of CD4 or CD8 cell surface marker expression. As shown in FIG. 32, the results indicated that CAR transduction promoted T cell maturation and that an increased degree of T cell maturation was observed in the pro-T cells in comparison to bone marrow (BM)-derived CD34+ cells and the CD34+ cells derived from the embryonic bodies (EB).

The ability of LV-transduced pro-T cells to function via anti-CD19 receptor-mediated cytotoxicity was evaluated. The T cell subsets were cocultured with a CD19+ leukemia cell line (NALM6) expressing a luciferase reporter gene (Luc+) to measure the degree of T cell-mediated cell lysis, with untransduced cells and Pan T cells as a negative and positive control, respectively. As shown in FIG. 33, the CAR pro-T cells effectively functioned via T cell-mediated lysis, demonstrating a degree of cytotoxicity comparable to the CAR-pro T cells derived from the BM CD34+ cells. Conversely, the CAR pro-T cells derived from the EB CD34+ cells showed no ability to kill the target cells.

### Example 14—Evaluating HSC Properties of Developing into Pro-T Cells

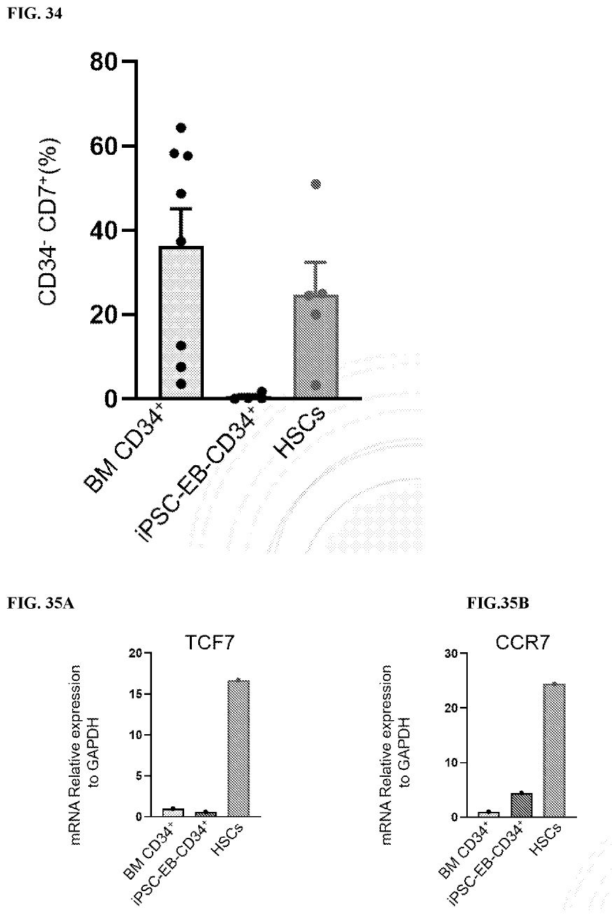

The ability of the HSCs to develop into pro-T cells was assessed by measuring the CD34-CD7+ markers on the pro-T cells. As shown in FIG. 34, FACS analysis showed that HSCs produced according to this disclosure successfully differentiated into CD34-CD7+ pro-T cells, as compared to bone marrow derived CD34+ cells or EB-derived CD34+ cells.

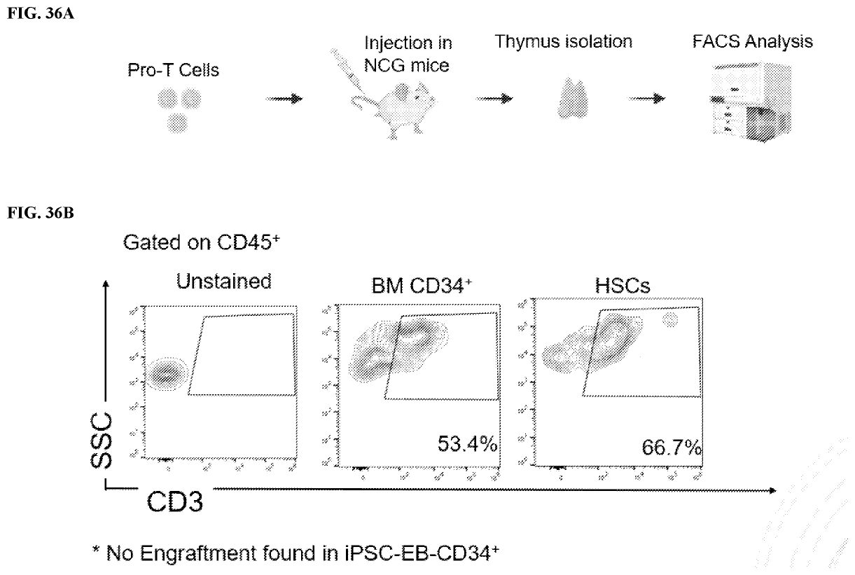

Next, the expression of T cell-specific transcription factors and thymus engrafting molecules were measured. FIG. 35A shows increased TCF7 expression and FIG. 35B shows increased CCR7 expression in the HSC-derived pro-T cells of the disclosure. FIG. 36A shows HSCs-derived Pro-T Cells engraft and differentiate in thymus. FIG. 36B shows FACS analysis of CD3 cell population of cells gated on a CD45+ cell population, which shows the superior engraftment and differentiation potential of the HSCs-derived Pro-T Cells in the thymus. Pro-T Cells of this example were prepared from HSCs using Piezol activation as already described.

1. An HLA-modified cell population that is HLA-A^{neg}, and is (1) HLA-DPA1^{neg} and/or HLA-DPB1^{neg}, and/or (2) HLA-DQA1^{neg} and/or HLA-DQB1^{neg}, the cell population being homozygous for or comprising a single gene for HLA-C, HLA-DRB1, and optionally HLA-B.

2. The cell population of claim 1, wherein the cell population is HLA-DPB1^{neg} and/or HLA-DQB1^{neg}.

3. The cell population of claim 2, wherein the cell population is HLA-DPB1^{neg} and HLA-DQB1^{neg}.

4. The cell population of claim 1, wherein the cell population comprises a deletion or inactivation of both DPB1 genes.

5. The cell population of claim 1, wherein one or both DQB1 genes are retained and optionally one or both DQA1 genes are retained.

6. The cell population of claim 1, wherein the cell population comprises a deletion or inactivation of both DQB1 genes, and optionally of both DQA1 genes.

7. The cell population of claim 6, wherein one or both DPB1 genes are retained and/or one or both DPA1 genes are retained.

8-20. (canceled)

21. The cell population of claim 1, wherein the cell is a human stem cell or human progenitor cell.

22. The cell population of claim 21, wherein the stem cell is a pluripotent stem cell, which is optionally a human induced pluripotent stem cell (hiPSC).

23. The cell population of claim 22, wherein the iPSCs are derived from cord blood, bone marrow biopsy for human HSCs, mobilized peripheral blood derived hCD34+ cells, human CD34+ cells, immune cells, immune progenitor cells, hematopoietic cells, non-hematopoietic cells, and banked organ derived cells.

24-28. (canceled)

29. The cell population of claim 1, wherein the cell population has a DRB1 haplotype selected from DRB1*03:01, DRB1*15:01, DRB1*01.02, DRB1*07.01, DRB1*04:01, DRB1*07:01, DRB1*01:01, DRB1*01:02, DRB1*04:04, DRB1*13:02, DRB1*13:01, DRB1*11:04, DRB1*15:02, DRB1*03:02, DRB1*11:01, DRB1*15:03, DRB1*04:07, DRB1*08:04, DRB1*04:02, DRB1*14:01, and DRB1*10:01.

30. The cell population of claim 1, wherein the cell population has an HLA-C haplotype selected from C*07:01, C*04:01, C*07:02, C*06:02, C*03:04, C*05:01, C*02:02, C*12:03, C*03:03, and C*16:01.

31. (canceled)

32. The cell population of claim 1, wherein the cell population has an HLA-B haplotype selected from B*08:01 and B*07:02.

33-34. (canceled)

35. A collection of cell population according to claim 1, wherein the cell populations in the collection represent at least two different HLA-C haplotypes.

36-71. (canceled)

72. A method for cell therapy, comprising, administering to a recipient in need thereof a cell population according to claim 1.

73-87. (canceled)

88. A method for making a cell population of claim 23, comprising:
providing an iPSC;
modifying the iPSC to prepare an HLA-modified iPSC population that is HLA-A^{neg}, and is (1) HLA-DPA1^{neg} and/or HLA-DPB1^{neg} and/or (2) HLA-DQA1^{neg} and/or HLA-DQB1^{neg}, the iPSC population being homozygous for or comprising a single gene for HLA-C, HLA-DRB1, and optionally HLA-B;
preparing embryoid bodies (EBs) from the iPSC population;
dissociating the EBs and enriching for CD34+ cells to prepare a CD34+-enriched cell population;
inducing endothelial-to-hematopoietic transition (EHT) of the CD34+-enriched cell population to prepare a population comprising hematopoietic stem cells (HSCs) and/or hematopoietic stem progenitor cells (HSPCs),
optionally harvesting CD34+ cells from the population comprising HSCs and/or HSPCs to enrich for a population undergoing EHT; and
optionally differentiating the cell population undergoing EHT to a hematopoietic lineage.

89-176. (canceled)


SEQUENCE LISTING (1473 sequences)

SEQ ID NO: 1 - DNA, 20 bp, synthetic construct
  source 1..20: mol_type = other DNA
gagggttcggggcgccatga

SEQ ID NO: 2 - DNA, 20 bp, synthetic construct
  source 1..20: mol_type = other DNA
gaagaaatacctcatggagt

SEQ ID NO: 3 - DNA, 20 bp, synthetic construct
  source 1..20: mol_type = other DNA
ggatgtgaagaaatacctca

SEQ ID NO: 4 - DNA, 20 bp, synthetic construct
  source 1..20: mol_type = other DNA
agtagcaggaggagggttcg

SEQ ID NO: 5 - DNA, 20 bp, synthetic construct
  source 1..20: mol_type = other DNA
tgaagaaatacctcatggag

SEQ ID NO: 6 - DNA, 20 bp, synthetic construct
  source 1..20: mol_type = other DNA
agtagcaggacgagggttcg

SEQ ID NO: 7 - DNA, 20 bp, synthetic construct
  source 1..20: mol_type = other DNA
atttcttcacatccgtgtcc

SEQ ID NO: 8 - DNA, 20 bp, synthetic construct
  source 1..20: mol_type = other DNA
gagtagcaggaggagggttc

SEQ ID NO: 9 - DNA, 20 bp, synthetic construct
  source 1..20: mol_type = other DNA
gagtagcaggacgagggttc

SEQ ID NO: 10 - DNA, 20 bp, synthetic construct
  source 1..20: mol_type = other DNA
ttcacatccgtgtcccggcc

SEQ ID NO: 11 - DNA, 20 bp, synthetic construct
  source 1..20: mol_type = other DNA
agagtagcaggaggagggtt

SEQ ID NO: 12 - DNA, 20 bp, synthetic construct
  source 1..20: mol_type = other DNA
agagtagcaggacgagggtt

SEQ ID NO: 13 - DNA, 20 bp, synthetic construct
  source 1..20: mol_type = other DNA
tccgtgtcccggcccggccg

SEQ ID NO: 14 - DNA, 20 bp, synthetic construct
  source 1..20: mol_type = other DNA
aaccctcctcctgctactct

SEQ ID NO: 15 - DNA, 20 bp, synthetic construct
  source 1..20: mol_type = other DNA
aaccctcgtcctgctactct

SEQ ID NO: 16 - DNA, 20 bp, synthetic construct
  source 1..20: mol_type = other DNA
ccgtgtcccggcccggccgc

SEQ ID NO: 17 - DNA, 20 bp, synthetic construct
  source 1..20: mol_type = other DNA
accctcctcctgctactctc

SEQ ID NO: 18 - DNA, 20 bp, synthetic construct
  source 1..20: mol_type = other DNA
accctcgtcctgctactctc

SEQ ID NO: 19 - DNA, 20 bp, synthetic construct
  source 1..20: mol_type = other DNA
cccgcggccgggccgggaca

SEQ ID NO: 20 - DNA, 20 bp, synthetic construct
  source 1..20: mol_type = other DNA
ccctcctcctgctactctcg

SEQ ID NO: 21 - DNA, 20 bp, synthetic construct
  source 1..20: mol_type = other DNA
ccctcgtcctgctactctcg

SEQ ID NO: 22 - DNA, 20 bp, synthetic construct
  source 1..20: mol_type = other DNA
cgtgtcccggcccggccgcg

SEQ ID NO: 23 - DNA, 20 bp, synthetic construct
  source 1..20: mol_type = other DNA
ccccgagagtagcaggagga

SEQ ID NO: 24 - DNA, 20 bp, synthetic construct
  source 1..20: mol_type = other DNA
ccccgagagtagcaggacga

SEQ ID NO: 25 - DNA, 20 bp, synthetic construct
  source 1..20: mol_type = other DNA
gggctccccgcggccgggcc

SEQ ID NO: 26 - DNA, 20 bp, synthetic construct
  source 1..20: mol_type = other DNA
cctcctcctgctactctcgg

SEQ ID NO: 27 - DNA, 20 bp, synthetic construct
  source 1..20: mol_type = other DNA
cctcgtcctgctactctcgg

SEQ ID NO: 28 - DNA, 20 bp, synthetic construct
  source 1..20: mol_type = other DNA
ggggctccccgcggccgggc

SEQ ID NO: 29 - DNA, 20 bp, synthetic construct
  source 1..20: mol_type = other DNA
cccccgagagtagcaggagg

SEQ ID NO: 30 - DNA, 20 bp, synthetic construct
  source 1..20: mol_type = other DNA
cccccgagagtagcaggacg

SEQ ID NO: 31 - DNA, 20 bp, synthetic construct
  source 1..20: mol_type = other DNA
aagcggggctccccgcggcc

SEQ ID NO: 32 - DNA, 20 bp, synthetic construct
  source 1..20: mol_type = other DNA
gggcccccgagagtagcagg

SEQ ID NO: 33 - DNA, 20 bp, synthetic construct
  source 1..20: mol_type = other DNA
cctgctactctcgggggctc

SEQ ID NO: 34 - DNA, 20 bp, synthetic construct
  source 1..20: mol_type = other DNA
gaagcggggctccccgcggc

SEQ ID NO: 35 - DNA, 20 bp, synthetic construct
  source 1..20: mol_type = other DNA
cctgctactctcgggggccc

SEQ ID NO: 36 - DNA, 20 bp, synthetic construct
  source 1..20: mol_type = other DNA
ccagagcccccgagagtagc

SEQ ID NO: 37 - DNA, 20 bp, synthetic construct
  source 1..20: mol_type = other DNA
cgatgaagcggggctccccg

SEQ ID NO: 38 - DNA, 20 bp, synthetic construct
  source 1..20: mol_type = other DNA
ccagggcccccgagagtagc

SEQ ID NO: 39 - DNA, 20 bp, synthetic construct
  source 1..20: mol_type = other DNA
ctctggccctgacccagacc

SEQ ID NO: 40 - DNA, 20 bp, synthetic construct
  source 1..20: mol_type = other DNA
ggagccccgcttcatcgcag

SEQ ID NO: 41 - DNA, 20 bp, synthetic construct
  source 1..20: mol_type = other DNA
ccctggccctgacccagacc

SEQ ID NO: 42 - DNA, 20 bp, synthetic construct
  source 1..20: mol_type = other DNA
tctggccctgacccagacct

SEQ ID NO: 43 - DNA, 20 bp, synthetic construct
  source 1..20: mol_type = other DNA
gagccccgcttcatcgcagt

SEQ ID NO: 44 - DNA, 20 bp, synthetic construct
  source 1..20: mol_type = other DNA
ccaggtctgggtcagggcca

SEQ ID NO: 45 - DNA, 20 bp, synthetic construct
  source 1..20: mol_type = other DNA
ggagccccgcttcatcgccg

SEQ ID NO: 46 - DNA, 20 bp, synthetic construct
  source 1..20: mol_type = other DNA
ggccctgacccagacctggg

SEQ ID NO: 47 - DNA, 20 bp, synthetic construct
  source 1..20: mol_type = other DNA
tagcccactgcgatgaagcg

SEQ ID NO: 48 - DNA, 20 bp, synthetic construct
  source 1..20: mol_type = other DNA
cctggccctgacccagacct

SEQ ID NO: 49 - DNA, 20 bp, synthetic construct
  source 1..20: mol_type = other DNA
gagccccgcttcatcgccgt

SEQ ID NO: 50 - DNA, 20 bp, synthetic construct
  source 1..20: mol_type = other DNA
gtagcccactgcgatgaagc

SEQ ID NO: 51 - DNA, 20 bp, synthetic construct
  source 1..20: mol_type = other DNA
cccaggtctgggtcagggcc

SEQ ID NO: 52 - DNA, 20 bp, synthetic construct
  source 1..20: mol_type = other DNA
tagcccacggcgatgaagcg

SEQ ID NO: 53 - DNA, 20 bp, synthetic construct
  source 1..20: mol_type = other DNA
cgtagcccactgcgatgaag

SEQ ID NO: 54 - DNA, 20 bp, synthetic construct
  source 1..20: mol_type = other DNA
gtagcccacggcgatgaagc

SEQ ID NO: 55 - DNA, 20 bp, synthetic construct
  source 1..20: mol_type = other DNA
cttcatcgcagtgggctacg

SEQ ID NO: 56 - DNA, 20 bp, synthetic construct
  source 1..20: mol_type = other DNA
cgtagcccacggcgatgaag

SEQ ID NO: 57 - DNA, 20 bp, synthetic construct
  source 1..20: mol_type = other DNA
tggacgacacgcagttcgtg

SEQ ID NO: 58 - DNA, 20 bp, synthetic construct
  source 1..20: mol_type = other DNA
cttcatcgccgtgggctacg

SEQ ID NO: 59 - DNA, 20 bp, synthetic construct
  source 1..20: mol_type = other DNA
acagcgacgccgcgagccag

SEQ ID NO: 60 - DNA, 20 bp, synthetic construct
  source 1..20: mol_type = other DNA
cgtgtcgtccacgtagccca

SEQ ID NO: 61 - DNA, 20 bp, synthetic construct
  source 1..20: mol_type = other DNA
cgacgccgcgagccagagga

SEQ ID NO: 62 - DNA, 20 bp, synthetic construct
  source 1..20: mol_type = other DNA
cggctccatcctctggctcg

SEQ ID NO: 63 - DNA, 20 bp, synthetic construct
  source 1..20: mol_type = other DNA
cgacgccgcgagccagaaga

SEQ ID NO: 64 - DNA, 20 bp, synthetic construct
  source 1..20: mol_type = other DNA
cgagccagaggatggagccg

SEQ ID NO: 65 - DNA, 20 bp, synthetic construct
  source 1..20: mol_type = other DNA
cggctccatcttctggctcg

SEQ ID NO: 66 - DNA, 20 bp, synthetic construct
  source 1..20: mol_type = other DNA
gagccagaggatggagccgc

SEQ ID NO: 67 - DNA, 20 bp, synthetic construct
  source 1..20: mol_type = other DNA
cgagccagaagatggagccg

SEQ ID NO: 68 - DNA, 20 bp, synthetic construct
  source 1..20: mol_type = other DNA
gcgcccgcggctccatcctc

SEQ ID NO: 69 - DNA, 20 bp, synthetic construct
  source 1..20: mol_type = other DNA
gagccagaagatggagccgc

SEQ ID NO: 70 - DNA, 20 bp, synthetic construct
  source 1..20: mol_type = other DNA
ggatggagccgcgggcgccg

SEQ ID NO: 71 - DNA, 20 bp, synthetic construct
  source 1..20: mol_type = other DNA
gcgcccgcggctccatcttc

SEQ ID NO: 72 - DNA, 20 bp, synthetic construct
  source 1..20: mol_type = other DNA
tgctctatccacggcgcccg

SEQ ID NO: 73 - DNA, 20 bp, synthetic construct
  source 1..20: mol_type = other DNA
agatggagccgcgggcgccg

SEQ ID NO: 74 - DNA, 20 bp, synthetic construct
  source 1..20: mol_type = other DNA
gcgggcgccgtggatagagc

SEQ ID NO: 75 - DNA, 20 bp, synthetic construct
  source 1..20: mol_type = other DNA
ggcgccgtggatagagcagg

SEQ ID NO: 76 - DNA, 20 bp, synthetic construct
  source 1..20: mol_type = other DNA
gcgccgtggatagagcagga

SEQ ID NO: 77 - DNA, 20 bp, synthetic construct
  source 1..20: mol_type = other DNA
ggaccctcctgctctatcca

SEQ ID NO: 78 - DNA, 20 bp, synthetic construct
  source 1..20: mol_type = other DNA
gtggatagagcaggagggtc

SEQ ID NO: 79 - DNA, 20 bp, synthetic construct
  source 1..20: mol_type = other DNA
cgccgtggatagagcaggag

SEQ ID NO: 80 - DNA, 20 bp, synthetic construct
  source 1..20: mol_type = other DNA
agcaggagggtccggagtat

SEQ ID NO: 81 - DNA, 20 bp, synthetic construct
  source 1..20: mol_type = other DNA
ggcccctcctgctctatcca

SEQ ID NO: 82 - DNA, 20 bp, synthetic construct
  source 1..20: mol_type = other DNA
gcaggagggtccggagtatt

SEQ ID NO: 83 - DNA, 20 bp, synthetic construct
  source 1..20: mol_type = other DNA
gtggatagagcaggaggggc

SEQ ID NO: 84 - DNA, 20 bp, synthetic construct
  source 1..20: mol_type = other DNA
gagggtccggagtattggga

SEQ ID NO: 85 - DNA, 20 bp, synthetic construct
  source 1..20: mol_type = other DNA
agcaggaggggccggagtat

SEQ ID NO: 86 - DNA, 20 bp, synthetic construct
  source 1..20: mol_type = other DNA
agggtccggagtattgggac

SEQ ID NO: 87 - DNA, 20 bp, synthetic construct
  source 1..20: mol_type = other DNA
gcaggaggggccggagtatt

SEQ ID NO: 88 - DNA, 20 bp, synthetic construct
  source 1..20: mol_type = other DNA
gggtccggagtattgggacg

SEQ ID NO: 89 - DNA, 20 bp, synthetic construct
  source 1..20: mol_type = other DNA
ggggccggagtattgggacc

SEQ ID NO: 90 - DNA, 20 bp, synthetic construct
  source 1..20: mol_type = other DNA
gtctccccgtcccaatactc

SEQ ID NO: 91 - DNA, 20 bp, synthetic construct
  source 1..20: mol_type = other DNA
gtctcctggtcccaatactc

SEQ ID NO: 92 - DNA, 20 bp, synthetic construct
  source 1..20: mol_type = other DNA
agtattgggacggggagaca

SEQ ID NO: 93 - DNA, 20 bp, synthetic construct
  source 1..20: mol_type = other DNA
agtattgggaccaggagaca

SEQ ID NO: 94 - DNA, 20 bp, synthetic construct
  source 1..20: mol_type = other DNA
cggggagacacggaaagtga

SEQ ID NO: 95 - DNA, 20 bp, synthetic construct
  source 1..20: mol_type = other DNA
ccaggagacacggaatatga

SEQ ID NO: 96 - DNA, 20 bp, synthetic construct
  source 1..20: mol_type = other DNA
cactcggtgagtctgtgagt

SEQ ID NO: 97 - DNA, 20 bp, synthetic construct
  source 1..20: mol_type = other DNA
ccttcatattccgtgtctcc

SEQ ID NO: 98 - DNA, 20 bp, synthetic construct
  source 1..20: mol_type = other DNA
ccactcacagactcaccgag

SEQ ID NO: 99 - DNA, 20 bp, synthetic construct
  source 1..20: mol_type = other DNA
cgctcggtcagtctgtgagt

SEQ ID NO: 100 - DNA, 20 bp, synthetic construct
  source 1..20: mol_type = other DNA
ccactcggtgagtctgtgag

SEQ ID NO: 101 - DNA, 20 bp, synthetic construct
  source 1..20: mol_type = other DNA
tcgctcggtcagtctgtgag

SEQ ID NO: 102 - DNA, 20 bp, synthetic construct
  source 1..20: mol_type = other DNA
acagactcaccgagtggacc

SEQ ID NO: 103 - DNA, 20 bp, synthetic construct
  source 1..20: mol_type = other DNA
acagactgaccgagcgaacc

SEQ ID NO: 104 - DNA, 20 bp, synthetic construct
  source 1..20: mol_type = other DNA
cagactcaccgagtggacct

SEQ ID NO: 105 - DNA, 20 bp, synthetic construct
  source 1..20: mol_type = other DNA
cagactgaccgagcgaacct

SEQ ID NO: 106 - DNA, 20 bp, synthetic construct
  source 1..20: mol_type = other DNA
agactcaccgagtggacctg

SEQ ID NO: 107 - DNA, 20 bp, synthetic construct
  source 1..20: mol_type = other DNA
agactgaccgagcgaacctg

SEQ ID NO: 108 - DNA, 20 bp, synthetic construct
  source 1..20: mol_type = other DNA
gcagggtccccaggtccact

SEQ ID NO: 109 - DNA, 20 bp, synthetic construct
  source 1..20: mol_type = other DNA
gcagggtccccaggttcgct

SEQ ID NO: 110 - DNA, 20 bp, synthetic construct
  source 1..20: mol_type = other DNA
gtggacctggggaccctgcg

SEQ ID NO: 111 - DNA, 20 bp, synthetic construct
  source 1..20: mol_type = other DNA
gcgaacctggggaccctgcg

SEQ ID NO: 112 - DNA, 20 bp, synthetic construct
  source 1..20: mol_type = other DNA
agtagccgcgcagggtcccc

SEQ ID NO: 113 - DNA, 20 bp, synthetic construct
  source 1..20: mol_type = other DNA
ctggttgtagtagccgcgca

SEQ ID NO: 114 - DNA, 20 bp, synthetic construct
  source 1..20: mol_type = other DNA
tctggttgtagtagccgcgc

SEQ ID NO: 115 - DNA, 20 bp, synthetic construct
  source 1..20: mol_type = other DNA
cggctactacaaccagagcg

SEQ ID NO: 116 - DNA, 20 bp, synthetic construct
  source 1..20: mol_type = other DNA
ggaggtgtagaaatacctca

SEQ ID NO: 117 - DNA, 20 bp, synthetic construct
  source 1..20: mol_type = other DNA
atttctacacctccgtgtcc

SEQ ID NO: 118 - DNA, 20 bp, synthetic construct
  source 1..20: mol_type = other DNA
tacacctccgtgtcccggcc

SEQ ID NO: 119 - DNA, 20 bp, synthetic construct
  source 1..20: mol_type = other DNA
gcggccgggccgggacacgg

SEQ ID NO: 120 - DNA, 20 bp, synthetic construct
  source 1..20: mol_type = other DNA
tcgacagcgacgccgcgagc

SEQ ID NO: 121 - DNA, 20 bp, synthetic construct
  source 1..20: mol_type = other DNA
acagcgacgccgcgagccgg

SEQ ID NO: 122 - DNA, 20 bp, synthetic construct
  source 1..20: mol_type = other DNA
cgacgccgcgagccggagga

SEQ ID NO: 123 - DNA, 20 bp, synthetic construct
  source 1..20: mol_type = other DNA
cggctccatcctccggctcg

SEQ ID NO: 124 - DNA, 20 bp, synthetic construct
  source 1..20: mol_type = other DNA
cgagccggaggatggagccg

SEQ ID NO: 125 - DNA, 20 bp, synthetic construct
  source 1..20: mol_type = other DNA
gagccggaggatggagccgc

SEQ ID NO: 126 - DNA, 20 bp, synthetic construct
  source 1..20: mol_type = other DNA
ccaggagacacggaatgtga

SEQ ID NO: 127 - DNA, 20 bp, synthetic construct
  source 1..20: mol_type = other DNA
ccttcacattccgtgtctcc

SEQ ID NO: 128 - DNA, 20 bp, synthetic construct
  source 1..20: mol_type = other DNA
cactcggtcagtctgtgact

SEQ ID NO: 129 - DNA, 20 bp, synthetic construct
  source 1..20: mol_type = other DNA
ccagtcacagactgaccgag

SEQ ID NO: 130 - DNA, 20 bp, synthetic construct
  source 1..20: mol_type = other DNA
ccactcggtcagtctgtgac

SEQ ID NO: 131 - DNA, 20 bp, synthetic construct
  source 1..20: mol_type = other DNA
acagactgaccgagtggacc

SEQ ID NO: 132 - DNA, 20 bp, synthetic construct
  source 1..20: mol_type = other DNA
cagactgaccgagtggacct

SEQ ID NO: 133 - DNA, 20 bp, synthetic construct
  source 1..20: mol_type = other DNA
agactgaccgagtggacctg

SEQ ID NO: 134 - DNA, 20 bp, synthetic construct
  source 1..20: mol_type = other DNA
gtagaaatacctcatggagt

SEQ ID NO: 135 - DNA, 20 bp, synthetic construct
  source 1..20: mol_type = other DNA
ggagaaatacctcatggagt

SEQ ID NO: 136 - DNA, 20 bp, synthetic construct
  source 1..20: mol_type = other DNA
tgtagaaatacctcatggag

SEQ ID NO: 137 - DNA, 20 bp, synthetic construct
  source 1..20: mol_type = other DNA
tggagaaatacctcatggag

SEQ ID NO: 138 - DNA, 20 bp, synthetic construct
  source 1..20: mol_type = other DNA
ggatgtggagaaatacctca

SEQ ID NO: 139 - DNA, 20 bp, synthetic construct
  source 1..20: mol_type = other DNA
atttctccacatccgtgtcc

SEQ ID NO: 140 - DNA, 20 bp, synthetic construct
  source 1..20: mol_type = other DNA
tccacatccgtgtcccggcc

SEQ ID NO: 141 - DNA, 20 bp, synthetic construct
  source 1..20: mol_type = other DNA
gccgggccgggacacggatg

SEQ ID NO: 142 - DNA, 20 bp, synthetic construct
  source 1..20: mol_type = other DNA
ggggccggagtattgggacg

SEQ ID NO: 143 - DNA, 20 bp, synthetic construct
  source 1..20: mol_type = other DNA
gtctcctcgtcccaatactc

SEQ ID NO: 144 - DNA, 20 bp, synthetic construct
  source 1..20: mol_type = other DNA
gagtattgggacgaggagac

SEQ ID NO: 145 - DNA, 20 bp, synthetic construct
  source 1..20: mol_type = other DNA
agtattgggacgaggagaca

SEQ ID NO: 146 - DNA, 20 bp, synthetic construct
  source 1..20: mol_type = other DNA
cgaggagacagggaaagtga

SEQ ID NO: 147 - DNA, 20 bp, synthetic construct
  source 1..20: mol_type = other DNA
ctctcggtcagtctgtgagt

SEQ ID NO: 148 - DNA, 20 bp, synthetic construct
  source 1..20: mol_type = other DNA
tctctcggtcagtctgtgag

SEQ ID NO: 149 - DNA, 20 bp, synthetic construct
  source 1..20: mol_type = other DNA
agactgaccgagagaacctg

SEQ ID NO: 150 - DNA, 20 bp, synthetic construct
  source 1..20: mol_type = other DNA
gcgcgatccgcaggttctct

SEQ ID NO: 151 - DNA, 20 bp, synthetic construct
  source 1..20: mol_type = other DNA
agtagcggagcgcgatccgc

SEQ ID NO: 152 - DNA, 20 bp, synthetic construct
  source 1..20: mol_type = other DNA
ccgctactacaaccagagcg

SEQ ID NO: 153 - DNA, 20 bp, synthetic construct
  source 1..20: mol_type = other DNA
cctcgctctggttgtagtag

SEQ ID NO: 154 - DNA, 20 bp, synthetic construct
  source 1..20: mol_type = other DNA
aggggccggagtattgggac

SEQ ID NO: 155 - DNA, 20 bp, synthetic construct
  source 1..20: mol_type = other DNA
gtgttccggtcccaatactc

SEQ ID NO: 156 - DNA, 20 bp, synthetic construct
  source 1..20: mol_type = other DNA
agtattgggaccggaacaca

SEQ ID NO: 157 - DNA, 20 bp, synthetic construct
  source 1..20: mol_type = other DNA
ccggaacacacggaatgtga

SEQ ID NO: 158 - DNA, 20 bp, synthetic construct
  source 1..20: mol_type = other DNA
ccttcacattccgtgtgttc

SEQ ID NO: 159 - DNA, 20 bp, synthetic construct
  source 1..20: mol_type = other DNA
agactgaccgagagagcctg

SEQ ID NO: 160 - DNA, 20 bp, synthetic construct
  source 1..20: mol_type = other DNA
gcgcgatccgcaggctctct

SEQ ID NO: 161 - DNA, 20 bp, synthetic construct
  source 1..20: mol_type = other DNA
ggtgaaatacctcatggagt

SEQ ID NO: 162 - DNA, 20 bp, synthetic construct
  source 1..20: mol_type = other DNA
tggtgaaatacctcatggag

SEQ ID NO: 163 - DNA, 20 bp, synthetic construct
  source 1..20: mol_type = other DNA
ggatgtggtgaaatacctca

SEQ ID NO: 164 - DNA, 20 bp, synthetic construct
  source 1..20: mol_type = other DNA
atttcaccacatccgtgtcc

SEQ ID NO: 165 - DNA, 20 bp, synthetic construct
  source 1..20: mol_type = other DNA
accacatccgtgtcccggcc

SEQ ID NO: 166 - DNA, 20 bp, synthetic construct
  source 1..20: mol_type = other DNA
accctcctcctgctactctt

SEQ ID NO: 167 - DNA, 20 bp, synthetic construct
  source 1..20: mol_type = other DNA
ccctcctcctgctactcttg

SEQ ID NO: 168 - DNA, 20 bp, synthetic construct
  source 1..20: mol_type = other DNA
tccgtgtcccggcccggcag

SEQ ID NO: 169 - DNA, 20 bp, synthetic construct
  source 1..20: mol_type = other DNA
ccccaagagtagcaggagga

SEQ ID NO: 170 - DNA, 20 bp, synthetic construct
  source 1..20: mol_type = other DNA
tccactgccgggccgggaca

SEQ ID NO: 171 - DNA, 20 bp, synthetic construct
  source 1..20: mol_type = other DNA
cctcctcctgctactcttgg

SEQ ID NO: 172 - DNA, 20 bp, synthetic construct
  source 1..20: mol_type = other DNA
gggctctccactgccgggcc

SEQ ID NO: 173 - DNA, 20 bp, synthetic construct
  source 1..20: mol_type = other DNA
cccccaagagtagcaggagg

SEQ ID NO: 174 - DNA, 20 bp, synthetic construct
  source 1..20: mol_type = other DNA
ggggctctccactgccgggc

SEQ ID NO: 175 - DNA, 20 bp, synthetic construct
  source 1..20: mol_type = other DNA
gggcccccaagagtagcagg

SEQ ID NO: 176 - DNA, 20 bp, synthetic construct
  source 1..20: mol_type = other DNA
aagcggggctctccactgcc

SEQ ID NO: 177 - DNA, 20 bp, synthetic construct
  source 1..20: mol_type = other DNA
cctgctactcttgggggccc

SEQ ID NO: 178 - DNA, 20 bp, synthetic construct
  source 1..20: mol_type = other DNA
gaagcggggctctccactgc

SEQ ID NO: 179 - DNA, 20 bp, synthetic construct
  source 1..20: mol_type = other DNA
ccagggcccccaagagtagc

SEQ ID NO: 180 - DNA, 20 bp, synthetic construct
  source 1..20: mol_type = other DNA
agagccccgcttcatcgcag

SEQ ID NO: 181 - DNA, 20 bp, synthetic construct
  source 1..20: mol_type = other DNA
gtgcccgcggctccatcctc

SEQ ID NO: 182 - DNA, 20 bp, synthetic construct
  source 1..20: mol_type = other DNA
ggatggagccgcgggcaccg

SEQ ID NO: 183 - DNA, 20 bp, synthetic construct
  source 1..20: mol_type = other DNA
ggcctctcctgctctatcca

SEQ ID NO: 184 - DNA, 20 bp, synthetic construct
  source 1..20: mol_type = other DNA
tgctctatccacggtgcccg

SEQ ID NO: 185 - DNA, 20 bp, synthetic construct
  source 1..20: mol_type = other DNA
agcaggagaggcctgagtat

SEQ ID NO: 186 - DNA, 20 bp, synthetic construct
  source 1..20: mol_type = other DNA
gcgggcaccgtggatagagc

SEQ ID NO: 187 - DNA, 20 bp, synthetic construct
  source 1..20: mol_type = other DNA
gcaggagaggcctgagtatt

SEQ ID NO: 188 - DNA, 20 bp, synthetic construct
  source 1..20: mol_type = other DNA
ggcaccgtggatagagcagg

SEQ ID NO: 189 - DNA, 20 bp, synthetic construct
  source 1..20: mol_type = other DNA
gaggcctgagtattgggacc

SEQ ID NO: 190 - DNA, 20 bp, synthetic construct
  source 1..20: mol_type = other DNA
gcaccgtggatagagcagga

SEQ ID NO: 191 - DNA, 20 bp, synthetic construct
  source 1..20: mol_type = other DNA
caccgtggatagagcaggag

SEQ ID NO: 192 - DNA, 20 bp, synthetic construct
  source 1..20: mol_type = other DNA
gtctgcaggtcccaatactc

SEQ ID NO: 193 - DNA, 20 bp, synthetic construct
  source 1..20: mol_type = other DNA
agtattgggacctgcagaca

SEQ ID NO: 194 - DNA, 20 bp, synthetic construct
  source 1..20: mol_type = other DNA
cctgcagacacggaatgtga

SEQ ID NO: 195 - DNA, 20 bp, synthetic construct
  source 1..20: mol_type = other DNA
ccttcacattccgtgtctgc

SEQ ID NO: 196 - DNA, 20 bp, synthetic construct
  source 1..20: mol_type = other DNA
cgctcggtcagtctgtgact

SEQ ID NO: 197 - DNA, 20 bp, synthetic construct
  source 1..20: mol_type = other DNA
tcgctcggtcagtctgtgac

SEQ ID NO: 198 - DNA, 20 bp, synthetic construct
  source 1..20: mol_type = other DNA
acagactgaccgagagaacc

SEQ ID NO: 199 - DNA, 20 bp, synthetic construct
  source 1..20: mol_type = other DNA
cagactgaccgagagaacct

SEQ ID NO: 200 - DNA, 20 bp, synthetic construct
  source 1..20: mol_type = other DNA
cactcggtcaatctgtgagt

SEQ ID NO: 201 - DNA, 20 bp, synthetic construct
  source 1..20: mol_type = other DNA
gcagggtccccaggttctct

SEQ ID NO: 202 - DNA, 20 bp, synthetic construct
  source 1..20: mol_type = other DNA
ccactcacagattgaccgag

SEQ ID NO: 203 - DNA, 20 bp, synthetic construct
  source 1..20: mol_type = other DNA
gagaacctggggaccctgcg

SEQ ID NO: 204 - DNA, 20 bp, synthetic construct
  source 1..20: mol_type = other DNA
ccactcggtcaatctgtgag

SEQ ID NO: 205 - DNA, 20 bp, synthetic construct
  source 1..20: mol_type = other DNA
acagattgaccgagtggacc

SEQ ID NO: 206 - DNA, 20 bp, synthetic construct
  source 1..20: mol_type = other DNA
cagattgaccgagtggacct

SEQ ID NO: 207 - DNA, 20 bp, synthetic construct
  source 1..20: mol_type = other DNA
agattgaccgagtggacctg

SEQ ID NO: 208 - DNA, 20 bp, synthetic construct
  source 1..20: mol_type = other DNA
tactacaaccagagcgaggc

SEQ ID NO: 209 - DNA, 20 bp, synthetic construct
  source 1..20: mol_type = other DNA
agcagcaggaggacggttcg

SEQ ID NO: 210 - DNA, 20 bp, synthetic construct
  source 1..20: mol_type = other DNA
gagcagcaggaggacggttc

SEQ ID NO: 211 - DNA, 20 bp, synthetic construct
  source 1..20: mol_type = other DNA
agagcagcaggaggacggtt

SEQ ID NO: 212 - DNA, 20 bp, synthetic construct
  source 1..20: mol_type = other DNA
aaccgtcctcctgctgctct

SEQ ID NO: 213 - DNA, 20 bp, synthetic construct
  source 1..20: mol_type = other DNA
cgccgagagcagcaggagga

SEQ ID NO: 214 - DNA, 20 bp, synthetic construct
  source 1..20: mol_type = other DNA
cgtcctcctgctgctctcgg

SEQ ID NO: 215 - DNA, 20 bp, synthetic construct
  source 1..20: mol_type = other DNA
gggccgccgagagcagcagg

SEQ ID NO: 216 - DNA, 20 bp, synthetic construct
  source 1..20: mol_type = other DNA
cctgctgctctcggcggccc

SEQ ID NO: 217 - DNA, 20 bp, synthetic construct
  source 1..20: mol_type = other DNA
ccagggccgccgagagcagc

SEQ ID NO: 218 - DNA, 20 bp, synthetic construct
  source 1..20: mol_type = other DNA
ccctggccctgaccgagacc

SEQ ID NO: 219 - DNA, 20 bp, synthetic construct
  source 1..20: mol_type = other DNA
ccaggtctcggtcagggcca

SEQ ID NO: 220 - DNA, 20 bp, synthetic construct
  source 1..20: mol_type = other DNA
gtcgaaatacctcatggagt

SEQ ID NO: 221 - DNA, 20 bp, synthetic construct
  source 1..20: mol_type = other DNA
tgtcgaaatacctcatggag

SEQ ID NO: 222 - DNA, 20 bp, synthetic construct
  source 1..20: mol_type = other DNA
ggcggtgtcgaaatacctca

SEQ ID NO: 223 - DNA, 20 bp, synthetic construct
  source 1..20: mol_type = other DNA
atttcgacaccgccatgtcc

SEQ ID NO: 224 - DNA, 20 bp, synthetic construct
  source 1..20: mol_type = other DNA
gacaccgccatgtcccggcc

SEQ ID NO: 225 - DNA, 20 bp, synthetic construct
  source 1..20: mol_type = other DNA
gcggccgggccgggacatgg

SEQ ID NO: 226 - DNA, 20 bp, synthetic construct
  source 1..20: mol_type = other DNA
gccatgtcccggcccggccg

SEQ ID NO: 227 - DNA, 20 bp, synthetic construct
  source 1..20: mol_type = other DNA
ccatgtcccggcccggccgc

SEQ ID NO: 228 - DNA, 20 bp, synthetic construct
  source 1..20: mol_type = other DNA
catgtcccggcccggccgcg

SEQ ID NO: 229 - DNA, 20 bp, synthetic construct
  source 1..20: mol_type = other DNA
cctggccctgaccgagacct

SEQ ID NO: 230 - DNA, 20 bp, synthetic construct
  source 1..20: mol_type = other DNA
cccaggtctcggtcagggcc

SEQ ID NO: 231 - DNA, 20 bp, synthetic construct
  source 1..20: mol_type = other DNA
agatgaagcggggctccccg

SEQ ID NO: 232 - DNA, 20 bp, synthetic construct
  source 1..20: mol_type = other DNA
ggagccccgcttcatctcag

SEQ ID NO: 233 - DNA, 20 bp, synthetic construct
  source 1..20: mol_type = other DNA
gagccccgcttcatctcagt

SEQ ID NO: 234 - DNA, 20 bp, synthetic construct
  source 1..20: mol_type = other DNA
tagcccactgagatgaagcg

SEQ ID NO: 235 - DNA, 20 bp, synthetic construct
  source 1..20: mol_type = other DNA
gtagcccactgagatgaagc

SEQ ID NO: 236 - DNA, 20 bp, synthetic construct
  source 1..20: mol_type = other DNA
cgtagcccactgagatgaag

SEQ ID NO: 237 - DNA, 20 bp, synthetic construct
  source 1..20: mol_type = other DNA
cttcatctcagtgggctacg

SEQ ID NO: 238 - DNA, 20 bp, synthetic construct
  source 1..20: mol_type = other DNA
tggacgacacccagttcgtg

SEQ ID NO: 239 - DNA, 20 bp, synthetic construct
  source 1..20: mol_type = other DNA
gctgtcgaacctcacgaact

SEQ ID NO: 240 - DNA, 20 bp, synthetic construct
  source 1..20: mol_type = other DNA
cgctgtcgaacctcacgaac

SEQ ID NO: 241 - DNA, 20 bp, synthetic construct
  source 1..20: mol_type = other DNA
cgacgccgcgagtccgagag

SEQ ID NO: 242 - DNA, 20 bp, synthetic construct
  source 1..20: mol_type = other DNA
cggctcctctctcggactcg

SEQ ID NO: 243 - DNA, 20 bp, synthetic construct
  source 1..20: mol_type = other DNA
cgagtccgagagaggagccg

SEQ ID NO: 244 - DNA, 20 bp, synthetic construct
  source 1..20: mol_type = other DNA
gagtccgagagaggagccgc

SEQ ID NO: 245 - DNA, 20 bp, synthetic construct
  source 1..20: mol_type = other DNA
ggcgcccgcggctcctctct

SEQ ID NO: 246 - DNA, 20 bp, synthetic construct
  source 1..20: mol_type = other DNA
gagaggagccgcgggcgccg

SEQ ID NO: 247 - DNA, 20 bp, synthetic construct
  source 1..20: mol_type = other DNA
ccggaacacacagatctaca

SEQ ID NO: 248 - DNA, 20 bp, synthetic construct
  source 1..20: mol_type = other DNA
ccttgtagatctgtgtgttc

SEQ ID NO: 249 - DNA, 20 bp, synthetic construct
  source 1..20: mol_type = other DNA
cacacagatctacaaggccc

SEQ ID NO: 250 - DNA, 20 bp, synthetic construct
  source 1..20: mol_type = other DNA
ctctcggtcagtctgtgcct

SEQ ID NO: 251 - DNA, 20 bp, synthetic construct
  source 1..20: mol_type = other DNA
tctctcggtcagtctgtgcc

SEQ ID NO: 252 - DNA, 20 bp, synthetic construct
  source 1..20: mol_type = other DNA
gcaggttccgcaggctctct

SEQ ID NO: 253 - DNA, 20 bp, synthetic construct
  source 1..20: mol_type = other DNA
gagagcctgcggaacctgcg

SEQ ID NO: 254 - DNA, 20 bp, synthetic construct
  source 1..20: mol_type = other DNA
agtagccgcgcaggttccgc

SEQ ID NO: 255 - DNA, 20 bp, synthetic construct
  source 1..20: mol_type = other DNA
tcttgaagatctgtgtgttc

SEQ ID NO: 256 - DNA, 20 bp, synthetic construct
  source 1..20: mol_type = other DNA
ctctcggtcagtctgtgtgt

SEQ ID NO: 257 - DNA, 20 bp, synthetic construct
  source 1..20: mol_type = other DNA
agcagcaggaggagggttcg

SEQ ID NO: 258 - DNA, 20 bp, synthetic construct
  source 1..20: mol_type = other DNA
gagcagcaggaggagggttc

SEQ ID NO: 259 - DNA, 20 bp, synthetic construct
  source 1..20: mol_type = other DNA
agagcagcaggaggagggtt

SEQ ID NO: 260 - DNA, 20 bp, synthetic construct
  source 1..20: mol_type = other DNA
gaaccctcctcctgctgctc

SEQ ID NO: 261 - DNA, 20 bp, synthetic construct
  source 1..20: mol_type = other DNA
aaccctcctcctgctgctct

SEQ ID NO: 262 - DNA, 20 bp, synthetic construct
  source 1..20: mol_type = other DNA
accctcctcctgctgctctg

SEQ ID NO: 263 - DNA, 20 bp, synthetic construct
  source 1..20: mol_type = other DNA
ccctcctcctgctgctctgg

SEQ ID NO: 264 - DNA, 20 bp, synthetic construct
  source 1..20: mol_type = other DNA
cccccagagcagcaggagga

SEQ ID NO: 265 - DNA, 20 bp, synthetic construct
  source 1..20: mol_type = other DNA
cctcctcctgctgctctggg

SEQ ID NO: 266 - DNA, 20 bp, synthetic construct
  source 1..20: mol_type = other DNA
ccccccagagcagcaggagg

SEQ ID NO: 267 - DNA, 20 bp, synthetic construct
  source 1..20: mol_type = other DNA
ctgccccccagagcagcagg

SEQ ID NO: 268 - DNA, 20 bp, synthetic construct
  source 1..20: mol_type = other DNA
cctgctgctctggggggcag

SEQ ID NO: 269 - DNA, 20 bp, synthetic construct
  source 1..20: mol_type = other DNA
ccactgccccccagagcagc

SEQ ID NO: 270 - DNA, 20 bp, synthetic construct
  source 1..20: mol_type = other DNA
cagtggccctgaccgagacc

SEQ ID NO: 271 - DNA, 20 bp, synthetic construct
  source 1..20: mol_type = other DNA
agtggccctgaccgagacct

SEQ ID NO: 272 - DNA, 20 bp, synthetic construct
  source 1..20: mol_type = other DNA
ggcggtgtagaaatacctca

SEQ ID NO: 273 - DNA, 20 bp, synthetic construct
  source 1..20: mol_type = other DNA
atttctacaccgccatgtcc

SEQ ID NO: 274 - DNA, 20 bp, synthetic construct
  source 1..20: mol_type = other DNA
tacaccgccatgtcccggcc

SEQ ID NO: 275 - DNA, 20 bp, synthetic construct
  source 1..20: mol_type = other DNA
tgatgaagcggggctccccg

SEQ ID NO: 276 - DNA, 20 bp, synthetic construct
  source 1..20: mol_type = other DNA
ggagccccgcttcatcaccg

SEQ ID NO: 277 - DNA, 20 bp, synthetic construct
  source 1..20: mol_type = other DNA
gagccccgcttcatcaccgt

SEQ ID NO: 278 - DNA, 20 bp, synthetic construct
  source 1..20: mol_type = other DNA
tagcccacggtgatgaagcg

SEQ ID NO: 279 - DNA, 20 bp, synthetic construct
  source 1..20: mol_type = other DNA
gtagcccacggtgatgaagc

SEQ ID NO: 280 - DNA, 20 bp, synthetic construct
  source 1..20: mol_type = other DNA
cgtagcccacggtgatgaag

SEQ ID NO: 281 - DNA, 20 bp, synthetic construct
  source 1..20: mol_type = other DNA
cttcatcaccgtgggctacg

SEQ ID NO: 282 - DNA, 20 bp, synthetic construct
  source 1..20: mol_type = other DNA
ggtgtcgtccacgtagccca

SEQ ID NO: 283 - DNA, 20 bp, synthetic construct
  source 1..20: mol_type = other DNA
acagcgacgccacgagtccg

SEQ ID NO: 284 - DNA, 20 bp, synthetic construct
  source 1..20: mol_type = other DNA
cgacgccacgagtccgagga

SEQ ID NO: 285 - DNA, 20 bp, synthetic construct
  source 1..20: mol_type = other DNA
gggcgccatcctcggactcg

SEQ ID NO: 286 - DNA, 20 bp, synthetic construct
  source 1..20: mol_type = other DNA
cgagtccgaggatggcgccc

SEQ ID NO: 287 - DNA, 20 bp, synthetic construct
  source 1..20: mol_type = other DNA
gagtccgaggatggcgcccc

SEQ ID NO: 288 - DNA, 20 bp, synthetic construct
  source 1..20: mol_type = other DNA
ggcgcccggggcgccatcct

SEQ ID NO: 289 - DNA, 20 bp, synthetic construct
  source 1..20: mol_type = other DNA
ggatggcgccccgggcgcca

SEQ ID NO: 290 - DNA, 20 bp, synthetic construct
  source 1..20: mol_type = other DNA
tgctctatccatggcgcccg

SEQ ID NO: 291 - DNA, 20 bp, synthetic construct
  source 1..20: mol_type = other DNA
ctgctctatccatggcgccc

SEQ ID NO: 292 - DNA, 20 bp, synthetic construct
  source 1..20: mol_type = other DNA
ccgggcgccatggatagagc

SEQ ID NO: 293 - DNA, 20 bp, synthetic construct
  source 1..20: mol_type = other DNA
cctgctctatccatggcgcc

SEQ ID NO: 294 - DNA, 20 bp, synthetic construct
  source 1..20: mol_type = other DNA
ggcgccatggatagagcagg

SEQ ID NO: 295 - DNA, 20 bp, synthetic construct
  source 1..20: mol_type = other DNA
gcgccatggatagagcagga

SEQ ID NO: 296 - DNA, 20 bp, synthetic construct
  source 1..20: mol_type = other DNA
cgccatggatagagcaggag

SEQ ID NO: 297 - DNA, 20 bp, synthetic construct
  source 1..20: mol_type = other DNA
atggatagagcaggaggggc

SEQ ID NO: 298 - DNA, 20 bp, synthetic construct
  source 1..20: mol_type = other DNA
gtctcccggtcccaatactc

SEQ ID NO: 299 - DNA, 20 bp, synthetic construct
  source 1..20: mol_type = other DNA
tcttggagatctgtgtctcc

SEQ ID NO: 300 - DNA, 20 bp, synthetic construct
  source 1..20: mol_type = other DNA
gtaagtctgtgtgttggtct

SEQ ID NO: 301 - DNA, 20 bp, synthetic construct
  source 1..20: mol_type = other DNA
ctctcggtaagtctgtgtgt

SEQ ID NO: 302 - DNA, 20 bp, synthetic construct
  source 1..20: mol_type = other DNA
gcgcggtgcgcaggttctct

SEQ ID NO: 303 - DNA, 20 bp, synthetic construct
  source 1..20: mol_type = other DNA
agtagcggagcgcggtgcgc

SEQ ID NO: 304 - DNA, 20 bp, synthetic construct
  source 1..20: mol_type = other DNA
ctggttgtagtagcggagcg

SEQ ID NO: 305 - DNA, 20 bp, synthetic construct
  source 1..20: mol_type = other DNA
atttctacaccgccgtgtcc

SEQ ID NO: 306 - DNA, 20 bp, synthetic construct
  source 1..20: mol_type = other DNA
tacaccgccgtgtcccggcc

SEQ ID NO: 307 - DNA, 20 bp, synthetic construct
  source 1..20: mol_type = other DNA
gccgtgtcccggcccggccg

SEQ ID NO: 308 - DNA, 20 bp, synthetic construct
  source 1..20: mol_type = other DNA
agcaggaggggccggaatat

SEQ ID NO: 309 - DNA, 20 bp, synthetic construct
  source 1..20: mol_type = other DNA
gcaggaggggccggaatatt

SEQ ID NO: 310 - DNA, 20 bp, synthetic construct
  source 1..20: mol_type = other DNA
aggggccggaatattgggac

SEQ ID NO: 311 - DNA, 20 bp, synthetic construct
  source 1..20: mol_type = other DNA
gtgttccggtcccaatattc

SEQ ID NO: 312 - DNA, 20 bp, synthetic construct
  source 1..20: mol_type = other DNA
tcttgcagatctgtgtgttc

SEQ ID NO: 313 - DNA, 20 bp, synthetic construct
  source 1..20: mol_type = other DNA
accgtcctcctgctgctctc

SEQ ID NO: 314 - DNA, 20 bp, synthetic construct
  source 1..20: mol_type = other DNA
tcccgagagcagcaggagga

SEQ ID NO: 315 - DNA, 20 bp, synthetic construct
  source 1..20: mol_type = other DNA
gggctcccgagagcagcagg

SEQ ID NO: 316 - DNA, 20 bp, synthetic construct
  source 1..20: mol_type = other DNA
cctgctgctctcgggagccc

SEQ ID NO: 317 - DNA, 20 bp, synthetic construct
  source 1..20: mol_type = other DNA
ccagggctcccgagagcagc

SEQ ID NO: 318 - DNA, 20 bp, synthetic construct
  source 1..20: mol_type = other DNA
acagcgacgccgcgagtccg

SEQ ID NO: 319 - DNA, 20 bp, synthetic construct
  source 1..20: mol_type = other DNA
cgacgccgcgagtccgagga

SEQ ID NO: 320 - DNA, 20 bp, synthetic construct
  source 1..20: mol_type = other DNA
agacttaccgagagagcctg

SEQ ID NO: 321 - DNA, 20 bp, synthetic construct
  source 1..20: mol_type = other DNA
atctcagtgggctacgtgga

SEQ ID NO: 322 - DNA, 20 bp, synthetic construct
  source 1..20: mol_type = other DNA
tggacggcacccagttcgtg

SEQ ID NO: 323 - DNA, 20 bp, synthetic construct
  source 1..20: mol_type = other DNA
gggctccgtcctcggactcg

SEQ ID NO: 324 - DNA, 20 bp, synthetic construct
  source 1..20: mol_type = other DNA
cgagtccgaggacggagccc

SEQ ID NO: 325 - DNA, 20 bp, synthetic construct
  source 1..20: mol_type = other DNA
gagtccgaggacggagcccc

SEQ ID NO: 326 - DNA, 20 bp, synthetic construct
  source 1..20: mol_type = other DNA
ggcgcccggggctccgtcct

SEQ ID NO: 327 - DNA, 20 bp, synthetic construct
  source 1..20: mol_type = other DNA
ggacggagccccgggcgccg

SEQ ID NO: 328 - DNA, 20 bp, synthetic construct
  source 1..20: mol_type = other DNA
ttgctctatccacggcgccc

SEQ ID NO: 329 - DNA, 20 bp, synthetic construct
  source 1..20: mol_type = other DNA
cttgctctatccacggcgcc

SEQ ID NO: 330 - DNA, 20 bp, synthetic construct
  source 1..20: mol_type = other DNA
ggcgccgtggatagagcaag

SEQ ID NO: 331 - DNA, 20 bp, synthetic construct
  source 1..20: mol_type = other DNA
gcgccgtggatagagcaaga

SEQ ID NO: 332 - DNA, 20 bp, synthetic construct
  source 1..20: mol_type = other DNA
cgccgtggatagagcaagag

SEQ ID NO: 333 - DNA, 20 bp, synthetic construct
  source 1..20: mol_type = other DNA
ggcccctcttgctctatcca

SEQ ID NO: 334 - DNA, 20 bp, synthetic construct
  source 1..20: mol_type = other DNA
gtggatagagcaagaggggc

SEQ ID NO: 335 - DNA, 20 bp, synthetic construct
  source 1..20: mol_type = other DNA
agcaagaggggccggagtat

SEQ ID NO: 336 - DNA, 20 bp, synthetic construct
  source 1..20: mol_type = other DNA
gcaagaggggccggagtatt

SEQ ID NO: 337 - DNA, 20 bp, synthetic construct
  source 1..20: mol_type = other DNA
tcttggagatctgtgtgttc

SEQ ID NO: 338 - DNA, 20 bp, synthetic construct
  source 1..20: mol_type = other DNA
caggacgaagtcccaggccc

SEQ ID NO: 339 - DNA, 20 bp, synthetic construct
  source 1..20: mol_type = other DNA
aggacgaagtcccaggcccc

SEQ ID NO: 340 - DNA, 20 bp, synthetic construct
  source 1..20: mol_type = other DNA
acgaagtcccaggccccggg

SEQ ID NO: 341 - DNA, 20 bp, synthetic construct
  source 1..20: mol_type = other DNA
cgaagtcccaggccccgggc

SEQ ID NO: 342 - DNA, 20 bp, synthetic construct
  source 1..20: mol_type = other DNA
gaagtcccaggccccgggcg

SEQ ID NO: 343 - DNA, 20 bp, synthetic construct
  source 1..20: mol_type = other DNA
gagagccccgcccggggcct

SEQ ID NO: 344 - DNA, 20 bp, synthetic construct
  source 1..20: mol_type = other DNA
tgagagccccgcccggggcc

SEQ ID NO: 345 - DNA, 20 bp, synthetic construct
  source 1..20: mol_type = other DNA
aggccccgggcggggctctc

SEQ ID NO: 346 - DNA, 20 bp, synthetic construct
  source 1..20: mol_type = other DNA
ggccccgggcggggctctca

SEQ ID NO: 347 - DNA, 20 bp, synthetic construct
  source 1..20: mol_type = other DNA
ggccccgggcggggctctca

SEQ ID NO: 348 - DNA, 20 bp, synthetic construct
  source 1..20: mol_type = other DNA
agaccctgagagccccgccc

SEQ ID NO: 349 - DNA, 20 bp, synthetic construct
  source 1..20: mol_type = other DNA
gagaccctgagagccccgcc

SEQ ID NO: 350 - DNA, 20 bp, synthetic construct
  source 1..20: mol_type = other DNA
ggcggggctctcagggtctc

SEQ ID NO: 351 - DNA, 20 bp, synthetic construct
  source 1..20: mol_type = other DNA
tccgagagccttgtctgcat

SEQ ID NO: 352 - DNA, 20 bp, synthetic construct
  source 1..20: mol_type = other DNA
ccgagagccttgtctgcatt

SEQ ID NO: 353 - DNA, 20 bp, synthetic construct
  source 1..20: mol_type = other DNA
cccaatgcagacaaggctct

SEQ ID NO: 354 - DNA, 20 bp, synthetic construct
  source 1..20: mol_type = other DNA
cgagagccttgtctgcattg

SEQ ID NO: 355 - DNA, 20 bp, synthetic construct
  source 1..20: mol_type = other DNA
gagccttgtctgcattgggg

SEQ ID NO: 356 - DNA, 20 bp, synthetic construct
  source 1..20: mol_type = other DNA
gcgcctccccaatgcagaca

SEQ ID NO: 357 - DNA, 20 bp, synthetic construct
  source 1..20: mol_type = other DNA
gcattggggaggcgcagcgt

SEQ ID NO: 358 - DNA, 20 bp, synthetic construct
  source 1..20: mol_type = other DNA
cattggggaggcgcagcgtt

SEQ ID NO: 359 - DNA, 20 bp, synthetic construct
  source 1..20: mol_type = other DNA
attggggaggcgcagcgttg

SEQ ID NO: 360 - DNA, 20 bp, synthetic construct
  source 1..20: mol_type = other DNA
aagtgaaactcgtgggagtg

SEQ ID NO: 361 - DNA, 20 bp, synthetic construct
  source 1..20: mol_type = other DNA
gaagtgaaactcgtgggagt

SEQ ID NO: 362 - DNA, 20 bp, synthetic construct
  source 1..20: mol_type = other DNA
agaagtgaaactcgtgggag

SEQ ID NO: 363 - DNA, 20 bp, synthetic construct
  source 1..20: mol_type = other DNA
ggagaagaagtgaaactcgt

SEQ ID NO: 364 - DNA, 20 bp, synthetic construct
  source 1..20: mol_type = other DNA
gggagaagaagtgaaactcg

SEQ ID NO: 365 - DNA, 20 bp, synthetic construct
  source 1..20: mol_type = other DNA
cttcttctcccaacctatgt

SEQ ID NO: 366 - DNA, 20 bp, synthetic construct
  source 1..20: mol_type = other DNA
ttcttctcccaacctatgtc

SEQ ID NO: 367 - DNA, 20 bp, synthetic construct
  source 1..20: mol_type = other DNA
agaaggacccgacataggtt

SEQ ID NO: 368 - DNA, 20 bp, synthetic construct
  source 1..20: mol_type = other DNA
aagaaggacccgacataggt

SEQ ID NO: 369 - DNA, 20 bp, synthetic construct
  source 1..20: mol_type = other DNA
ctggaagaaggacccgacat

SEQ ID NO: 370 - DNA, 20 bp, synthetic construct
  source 1..20: mol_type = other DNA
ctatgtcgggtccttcttcc

SEQ ID NO: 371 - DNA, 20 bp, synthetic construct
  source 1..20: mol_type = other DNA
gtcacgagtatcctggaaga

SEQ ID NO: 372 - DNA, 20 bp, synthetic construct
  source 1..20: mol_type = other DNA
gggacgcgtcacgagtatcc

SEQ ID NO: 373 - DNA, 20 bp, synthetic construct
  source 1..20: mol_type = other DNA
tccccatttcccactcccat

SEQ ID NO: 374 - DNA, 20 bp, synthetic construct
  source 1..20: mol_type = other DNA
ccccatttcccactcccatt

SEQ ID NO: 375 - DNA, 20 bp, synthetic construct
  source 1..20: mol_type = other DNA
cccaatgggagtgggaaatg

SEQ ID NO: 376 - DNA, 20 bp, synthetic construct
  source 1..20: mol_type = other DNA
acccaatgggagtgggaaat

SEQ ID NO: 377 - DNA, 20 bp, synthetic construct
  source 1..20: mol_type = other DNA
cacccaatgggagtgggaaa

SEQ ID NO: 378 - DNA, 20 bp, synthetic construct
  source 1..20: mol_type = other DNA
ttcccactcccattgggtgt

SEQ ID NO: 379 - DNA, 20 bp, synthetic construct
  source 1..20: mol_type = other DNA
atccgacacccaatgggagt

SEQ ID NO: 380 - DNA, 20 bp, synthetic construct
  source 1..20: mol_type = other DNA
tatccgacacccaatgggag

SEQ ID NO: 381 - DNA, 20 bp, synthetic construct
  source 1..20: mol_type = other DNA
ctagatatccgacacccaat

SEQ ID NO: 382 - DNA, 20 bp, synthetic construct
  source 1..20: mol_type = other DNA
tctagatatccgacacccaa

SEQ ID NO: 383 - DNA, 20 bp, synthetic construct
  source 1..20: mol_type = other DNA
gagaagccaatcagtgtcgc

SEQ ID NO: 384 - DNA, 20 bp, synthetic construct
  source 1..20: mol_type = other DNA
agaagccaatcagtgtcgcc

SEQ ID NO: 385 - DNA, 20 bp, synthetic construct
  source 1..20: mol_type = other DNA
gaagccaatcagtgtcgccg

SEQ ID NO: 386 - DNA, 20 bp, synthetic construct
  source 1..20: mol_type = other DNA
gggaccccggcgacactgat

SEQ ID NO: 387 - DNA, 20 bp, synthetic construct
  source 1..20: mol_type = other DNA
ggactttagaactgggaccc

SEQ ID NO: 388 - DNA, 20 bp, synthetic construct
  source 1..20: mol_type = other DNA
tgcgtggggactttagaact

SEQ ID NO: 389 - DNA, 20 bp, synthetic construct
  source 1..20: mol_type = other DNA
gtgcgtggggactttagaac

SEQ ID NO: 390 - DNA, 20 bp, synthetic construct
  source 1..20: mol_type = other DNA
aaagtccccacgcacccacc

SEQ ID NO: 391 - DNA, 20 bp, synthetic construct
  source 1..20: mol_type = other DNA
tgagtccgggtgggtgcgtg

SEQ ID NO: 392 - DNA, 20 bp, synthetic construct
  source 1..20: mol_type = other DNA
ctgagtccgggtgggtgcgt

SEQ ID NO: 393 - DNA, 20 bp, synthetic construct
  source 1..20: mol_type = other DNA
tctgagtccgggtgggtgcg

SEQ ID NO: 394 - DNA, 20 bp, synthetic construct
  source 1..20: mol_type = other DNA
aggagattctgagtccgggt

SEQ ID NO: 395 - DNA, 20 bp, synthetic construct
  source 1..20: mol_type = other DNA
gaggagattctgagtccggg

SEQ ID NO: 396 - DNA, 20 bp, synthetic construct
  source 1..20: mol_type = other DNA
tctgaggagattctgagtcc

SEQ ID NO: 397 - DNA, 20 bp, synthetic construct
  source 1..20: mol_type = other DNA
gtctgaggagattctgagtc

SEQ ID NO: 398 - DNA, 20 bp, synthetic construct
  source 1..20: mol_type = other DNA
tctcctcagacgccgagatg

SEQ ID NO: 399 - DNA, 20 bp, synthetic construct
  source 1..20: mol_type = other DNA
ctcctcagacgccgagatgc

SEQ ID NO: 400 - DNA, 20 bp, synthetic construct
  source 1..20: mol_type = other DNA
gacccgcatctcggcgtctg

SEQ ID NO: 401 - DNA, 20 bp, synthetic construct
  source 1..20: mol_type = other DNA
agacgccgagatgcgggtca

SEQ ID NO: 402 - DNA, 20 bp, synthetic construct
  source 1..20: mol_type = other DNA
gggcgccgtgacccgcatct

SEQ ID NO: 403 - DNA, 20 bp, synthetic construct
  source 1..20: mol_type = other DNA
gccgcgccgggaggagggtc

SEQ ID NO: 404 - DNA, 20 bp, synthetic construct
  source 1..20: mol_type = other DNA
gcccgaccctcctcccggcg

SEQ ID NO: 405 - DNA, 20 bp, synthetic construct
  source 1..20: mol_type = other DNA
gcgccgggaggagggtcggg

SEQ ID NO: 406 - DNA, 20 bp, synthetic construct
  source 1..20: mol_type = other DNA
cgccgggaggagggtcgggc

SEQ ID NO: 407 - DNA, 20 bp, synthetic construct
  source 1..20: mol_type = other DNA
gacccgcccgaccctcctcc

SEQ ID NO: 408 - DNA, 20 bp, synthetic construct
  source 1..20: mol_type = other DNA
ctcagcccctcctcgccccc

SEQ ID NO: 409 - DNA, 20 bp, synthetic construct
  source 1..20: mol_type = other DNA
gggagcctgggggcgaggag

SEQ ID NO: 410 - DNA, 20 bp, synthetic construct
  source 1..20: mol_type = other DNA
tgggagcctgggggcgagga

SEQ ID NO: 411 - DNA, 20 bp, synthetic construct
  source 1..20: mol_type = other DNA
gtgggagcctgggggcgagg

SEQ ID NO: 412 - DNA, 20 bp, synthetic construct
  source 1..20: mol_type = other DNA
ggagtgggagcctgggggcg

SEQ ID NO: 413 - DNA, 20 bp, synthetic construct
  source 1..20: mol_type = other DNA
ctcatggagtgggagcctgg

SEQ ID NO: 414 - DNA, 20 bp, synthetic construct
  source 1..20: mol_type = other DNA
ccccaggctcccactccatg

SEQ ID NO: 415 - DNA, 20 bp, synthetic construct
  source 1..20: mol_type = other DNA
cctcatggagtgggagcctg

SEQ ID NO: 416 - DNA, 20 bp, synthetic construct
  source 1..20: mol_type = other DNA
acctcatggagtgggagcct

SEQ ID NO: 417 - DNA, 20 bp, synthetic construct
  source 1..20: mol_type = other DNA
gtgacctgcgccccgggccg

SEQ ID NO: 418 - DNA, 20 bp, synthetic construct
  source 1..20: mol_type = other DNA
cgtgacctgcgccccgggcc

SEQ ID NO: 419 - DNA, 20 bp, synthetic construct
  source 1..20: mol_type = other DNA
tcgtgacctgcgccccgggc

SEQ ID NO: 420 - DNA, 20 bp, synthetic construct
  source 1..20: mol_type = other DNA
ggagtcgtgacctgcgcccc

SEQ ID NO: 421 - DNA, 20 bp, synthetic construct
  source 1..20: mol_type = other DNA
gggagtcgtgacctgcgccc

SEQ ID NO: 422 - DNA, 20 bp, synthetic construct
  source 1..20: mol_type = other DNA
gactccccatcccccacgta

SEQ ID NO: 423 - DNA, 20 bp, synthetic construct
  source 1..20: mol_type = other DNA
cgggccgtacgtgggggatg

SEQ ID NO: 424 - DNA, 20 bp, synthetic construct
  source 1..20: mol_type = other DNA
cccatcccccacgtacggcc

SEQ ID NO: 425 - DNA, 20 bp, synthetic construct
  source 1..20: mol_type = other DNA
ccgggccgtacgtgggggat

SEQ ID NO: 426 - DNA, 20 bp, synthetic construct
  source 1..20: mol_type = other DNA
ccatcccccacgtacggccc

SEQ ID NO: 427 - DNA, 20 bp, synthetic construct
  source 1..20: mol_type = other DNA
cccgggccgtacgtggggga

SEQ ID NO: 428 - DNA, 20 bp, synthetic construct
  source 1..20: mol_type = other DNA
gcgacccgggccgtacgtgg

SEQ ID NO: 429 - DNA, 20 bp, synthetic construct
  source 1..20: mol_type = other DNA
ggcgacccgggccgtacgtg

SEQ ID NO: 430 - DNA, 20 bp, synthetic construct
  source 1..20: mol_type = other DNA
gggcgacccgggccgtacgt

SEQ ID NO: 431 - DNA, 20 bp, synthetic construct
  source 1..20: mol_type = other DNA
ggggcgacccgggccgtacg

SEQ ID NO: 432 - DNA, 20 bp, synthetic construct
  source 1..20: mol_type = other DNA
cccgggtcgccccgagtctc

SEQ ID NO: 433 - DNA, 20 bp, synthetic construct
  source 1..20: mol_type = other DNA
ccggagactcggggcgaccc

SEQ ID NO: 434 - DNA, 20 bp, synthetic construct
  source 1..20: mol_type = other DNA
ccgggtcgccccgagtctcc

SEQ ID NO: 435 - DNA, 20 bp, synthetic construct
  source 1..20: mol_type = other DNA
cccggagactcggggcgacc

SEQ ID NO: 436 - DNA, 20 bp, synthetic construct
  source 1..20: mol_type = other DNA
cctcgaccggcgagagcccc

SEQ ID NO: 437 - DNA, 20 bp, synthetic construct
  source 1..20: mol_type = other DNA
cctggggctctcgccggtcg

SEQ ID NO: 438 - DNA, 20 bp, synthetic construct
  source 1..20: mol_type = other DNA
aacgcgcctggggctctcgc

SEQ ID NO: 439 - DNA, 20 bp, synthetic construct
  source 1..20: mol_type = other DNA
gagccccaggcgcgtttacc

SEQ ID NO: 440 - DNA, 20 bp, synthetic construct
  source 1..20: mol_type = other DNA
aaaccgggtaaacgcgcctg

SEQ ID NO: 441 - DNA, 20 bp, synthetic construct
  source 1..20: mol_type = other DNA
gaaaccgggtaaacgcgcct

SEQ ID NO: 442 - DNA, 20 bp, synthetic construct
  source 1..20: mol_type = other DNA
tgaaaccgggtaaacgcgcc

SEQ ID NO: 443 - DNA, 20 bp, synthetic construct
  source 1..20: mol_type = other DNA
cccggtttcattttcagttg

SEQ ID NO: 444 - DNA, 20 bp, synthetic construct
  source 1..20: mol_type = other DNA
cctcaactgaaaatgaaacc

SEQ ID NO: 445 - DNA, 20 bp, synthetic construct
  source 1..20: mol_type = other DNA
gcctcaactgaaaatgaaac

SEQ ID NO: 446 - DNA, 20 bp, synthetic construct
  source 1..20: mol_type = other DNA
cccgcgggttggtcggggcg

SEQ ID NO: 447 - DNA, 20 bp, synthetic construct
  source 1..20: mol_type = other DNA
ccccgccccgaccaacccgc

SEQ ID NO: 448 - DNA, 20 bp, synthetic construct
  source 1..20: mol_type = other DNA
gccccgccccgaccaacccg

SEQ ID NO: 449 - DNA, 20 bp, synthetic construct
  source 1..20: mol_type = other DNA
gcgggttggtcggggcgggg

SEQ ID NO: 450 - DNA, 20 bp, synthetic construct
  source 1..20: mol_type = other DNA
cgggttggtcggggcggggc

SEQ ID NO: 451 - DNA, 20 bp, synthetic construct
  source 1..20: mol_type = other DNA
gggttggtcggggcggggcg

SEQ ID NO: 452 - DNA, 20 bp, synthetic construct
  source 1..20: mol_type = other DNA
ggtcggggcggggcggggct

SEQ ID NO: 453 - DNA, 20 bp, synthetic construct
  source 1..20: mol_type = other DNA
gtcggggcggggcggggctc

SEQ ID NO: 454 - DNA, 20 bp, synthetic construct
  source 1..20: mol_type = other DNA
tcggggcggggcggggctcg

SEQ ID NO: 455 - DNA, 20 bp, synthetic construct
  source 1..20: mol_type = other DNA
cggggcggggcggggctcgg

SEQ ID NO: 456 - DNA, 20 bp, synthetic construct
  source 1..20: mol_type = other DNA
gcggggcggggctcggggga

SEQ ID NO: 457 - DNA, 20 bp, synthetic construct
  source 1..20: mol_type = other DNA
cggggcggggctcgggggac

SEQ ID NO: 458 - DNA, 20 bp, synthetic construct
  source 1..20: mol_type = other DNA
ggggcggggctcgggggacg

SEQ ID NO: 459 - DNA, 20 bp, synthetic construct
  source 1..20: mol_type = other DNA
tcgggggacggggctgaccg

SEQ ID NO: 460 - DNA, 20 bp, synthetic construct
  source 1..20: mol_type = other DNA
cgggggacggggctgaccgc

SEQ ID NO: 461 - DNA, 20 bp, synthetic construct
  source 1..20: mol_type = other DNA
gggggacggggctgaccgcg

SEQ ID NO: 462 - DNA, 20 bp, synthetic construct
  source 1..20: mol_type = other DNA
gacggggctgaccgcggggc

SEQ ID NO: 463 - DNA, 20 bp, synthetic construct
  source 1..20: mol_type = other DNA
ccgcggaggaggcgcccgtc

SEQ ID NO: 464 - DNA, 20 bp, synthetic construct
  source 1..20: mol_type = other DNA
ccgacgggcgcctcctccgc

SEQ ID NO: 465 - DNA, 20 bp, synthetic construct
  source 1..20: mol_type = other DNA
cccgcggaggaggcgcccgt

SEQ ID NO: 466 - DNA, 20 bp, synthetic construct
  source 1..20: mol_type = other DNA
actggtcatgcccgcggagg

SEQ ID NO: 467 - DNA, 20 bp, synthetic construct
  source 1..20: mol_type = other DNA
cggactggtcatgcccgcgg

SEQ ID NO: 468 - DNA, 20 bp, synthetic construct
  source 1..20: mol_type = other DNA
aggcggactggtcatgcccg

SEQ ID NO: 469 - DNA, 20 bp, synthetic construct
  source 1..20: mol_type = other DNA
catgaccagtccgcctacga

SEQ ID NO: 470 - DNA, 20 bp, synthetic construct
  source 1..20: mol_type = other DNA
ccagtccgcctacgacggca

SEQ ID NO: 471 - DNA, 20 bp, synthetic construct
  source 1..20: mol_type = other DNA
ccttgccgtcgtaggcggac

SEQ ID NO: 472 - DNA, 20 bp, synthetic construct
  source 1..20: mol_type = other DNA
gtaatccttgccgtcgtagg

SEQ ID NO: 473 - DNA, 20 bp, synthetic construct
  source 1..20: mol_type = other DNA
gatgtaatccttgccgtcgt

SEQ ID NO: 474 - DNA, 20 bp, synthetic construct
  source 1..20: mol_type = other DNA
ggattacatcgccctgaacg

SEQ ID NO: 475 - DNA, 20 bp, synthetic construct
  source 1..20: mol_type = other DNA
ggagctcaggtcctcgttca

SEQ ID NO: 476 - DNA, 20 bp, synthetic construct
  source 1..20: mol_type = other DNA
aggagctcaggtcctcgttc

SEQ ID NO: 477 - DNA, 20 bp, synthetic construct
  source 1..20: mol_type = other DNA
tgaacgaggacctgagctcc

SEQ ID NO: 478 - DNA, 20 bp, synthetic construct
  source 1..20: mol_type = other DNA
ggacctgagctcctggaccg

SEQ ID NO: 479 - DNA, 20 bp, synthetic construct
  source 1..20: mol_type = other DNA
ggagcagctgagagcctacc

SEQ ID NO: 480 - DNA, 20 bp, synthetic construct
  source 1..20: mol_type = other DNA
gcagctgagagcctacctgg

SEQ ID NO: 481 - DNA, 20 bp, synthetic construct
  source 1..20: mol_type = other DNA
cagctgagagcctacctgga

SEQ ID NO: 482 - DNA, 20 bp, synthetic construct
  source 1..20: mol_type = other DNA
cacgcacaggccctccaggt

SEQ ID NO: 483 - DNA, 20 bp, synthetic construct
  source 1..20: mol_type = other DNA
ctacctggagggcctgtgcg

SEQ ID NO: 484 - DNA, 20 bp, synthetic construct
  source 1..20: mol_type = other DNA
actccacgcacaggccctcc

SEQ ID NO: 485 - DNA, 20 bp, synthetic construct
  source 1..20: mol_type = other DNA
tggagggcctgtgcgtggag

SEQ ID NO: 486 - DNA, 20 bp, synthetic construct
  source 1..20: mol_type = other DNA
tgcggagccactccacgcac

SEQ ID NO: 487 - DNA, 20 bp, synthetic construct
  source 1..20: mol_type = other DNA
ggagtggctccgcagatacc

SEQ ID NO: 488 - DNA, 20 bp, synthetic construct
  source 1..20: mol_type = other DNA
ctccgcagatacctggagaa

SEQ ID NO: 489 - DNA, 20 bp, synthetic construct
  source 1..20: mol_type = other DNA
tccgcagatacctggagaac

SEQ ID NO: 490 - DNA, 20 bp, synthetic construct
  source 1..20: mol_type = other DNA
tcccgttctccaggtatctg

SEQ ID NO: 491 - DNA, 20 bp, synthetic construct
  source 1..20: mol_type = other DNA
cagatacctggagaacggga

SEQ ID NO: 492 - DNA, 20 bp, synthetic construct
  source 1..20: mol_type = other DNA
gcgtctccttcccgttctcc

SEQ ID NO: 493 - DNA, 20 bp, synthetic construct
  source 1..20: mol_type = other DNA
gaaggagacgctgcagcgcg

SEQ ID NO: 494 - DNA, 20 bp, synthetic construct
  source 1..20: mol_type = other DNA
aaggagacgctgcagcgcgc

SEQ ID NO: 495 - DNA, 20 bp, synthetic construct
  source 1..20: mol_type = other DNA
cgctgcagcgcgcgggtacc

SEQ ID NO: 496 - DNA, 20 bp, synthetic construct
  source 1..20: mol_type = other DNA
gctgcagcgcgcgggtacca

SEQ ID NO: 497 - DNA, 20 bp, synthetic construct
  source 1..20: mol_type = other DNA
ctgcagcgcgcgggtaccag

SEQ ID NO: 498 - DNA, 20 bp, synthetic construct
  source 1..20: mol_type = other DNA
ccacgagaagaggaggaaaa

SEQ ID NO: 499 - DNA, 20 bp, synthetic construct
  source 1..20: mol_type = other DNA
ccattttcctcctcttctcg

SEQ ID NO: 500 - DNA, 20 bp, synthetic construct
  source 1..20: mol_type = other DNA
cacgagaagaggaggaaaat

SEQ ID NO: 501 - DNA, 20 bp, synthetic construct
  source 1..20: mol_type = other DNA
gaatgtcgccctcccttgaa

SEQ ID NO: 502 - DNA, 20 bp, synthetic construct
  source 1..20: mol_type = other DNA
gccctcccttgaatggagaa

SEQ ID NO: 503 - DNA, 20 bp, synthetic construct
  source 1..20: mol_type = other DNA
gccattctccattcaaggga

SEQ ID NO: 504 - DNA, 20 bp, synthetic construct
  source 1..20: mol_type = other DNA
tgccattctccattcaaggg

SEQ ID NO: 505 - DNA, 20 bp, synthetic construct
  source 1..20: mol_type = other DNA
tcatgccattctccattcaa

SEQ ID NO: 506 - DNA, 20 bp, synthetic construct
  source 1..20: mol_type = other DNA
ctcatgccattctccattca

SEQ ID NO: 507 - DNA, 20 bp, synthetic construct
  source 1..20: mol_type = other DNA
gttttcctgagtttcctctg

SEQ ID NO: 508 - DNA, 20 bp, synthetic construct
  source 1..20: mol_type = other DNA
ttttcctgagtttcctctga

SEQ ID NO: 509 - DNA, 20 bp, synthetic construct
  source 1..20: mol_type = other DNA
ggggccctcagaggaaactc

SEQ ID NO: 510 - DNA, 20 bp, synthetic construct
  source 1..20: mol_type = other DNA
agagaagagggggccctcag

SEQ ID NO: 511 - DNA, 20 bp, synthetic construct
  source 1..20: mol_type = other DNA
gagggccccctcttctctct

SEQ ID NO: 512 - DNA, 20 bp, synthetic construct
  source 1..20: mol_type = other DNA
attgtcctagagagaagagg

SEQ ID NO: 513 - DNA, 20 bp, synthetic construct
  source 1..20: mol_type = other DNA
aattgtcctagagagaagag

SEQ ID NO: 514 - DNA, 20 bp, synthetic construct
  source 1..20: mol_type = other DNA
taattgtcctagagagaaga

SEQ ID NO: 515 - DNA, 20 bp, synthetic construct
  source 1..20: mol_type = other DNA
ttaattgtcctagagagaag

SEQ ID NO: 516 - DNA, 20 bp, synthetic construct
  source 1..20: mol_type = other DNA
tcttctctctaggacaatta

SEQ ID NO: 517 - DNA, 20 bp, synthetic construct
  source 1..20: mol_type = other DNA
cttctctctaggacaattaa

SEQ ID NO: 518 - DNA, 20 bp, synthetic construct
  source 1..20: mol_type = other DNA
attaagggatgacgtctctg

SEQ ID NO: 519 - DNA, 20 bp, synthetic construct
  source 1..20: mol_type = other DNA
ggatgacgtctctgaggaaa

SEQ ID NO: 520 - DNA, 20 bp, synthetic construct
  source 1..20: mol_type = other DNA
tgacgtctctgaggaaatgg

SEQ ID NO: 521 - DNA, 20 bp, synthetic construct
  source 1..20: mol_type = other DNA
gacgtctctgaggaaatgga

SEQ ID NO: 522 - DNA, 20 bp, synthetic construct
  source 1..20: mol_type = other DNA
acgtctctgaggaaatggag

SEQ ID NO: 523 - DNA, 20 bp, synthetic construct
  source 1..20: mol_type = other DNA
acttctgctcctgatctgag

SEQ ID NO: 524 - DNA, 20 bp, synthetic construct
  source 1..20: mol_type = other DNA
gagtctctgagcggggaaca

SEQ ID NO: 525 - DNA, 20 bp, synthetic construct
  source 1..20: mol_type = other DNA
cgagtctctgagcggggaac

SEQ ID NO: 526 - DNA, 20 bp, synthetic construct
  source 1..20: mol_type = other DNA
aaagttcgagtctctgagcg

SEQ ID NO: 527 - DNA, 20 bp, synthetic construct
  source 1..20: mol_type = other DNA
gaaagttcgagtctctgagc

SEQ ID NO: 528 - DNA, 20 bp, synthetic construct
  source 1..20: mol_type = other DNA
ggaaagttcgagtctctgag

SEQ ID NO: 529 - DNA, 20 bp, synthetic construct
  source 1..20: mol_type = other DNA
actcgaactttccaatgaat

SEQ ID NO: 530 - DNA, 20 bp, synthetic construct
  source 1..20: mol_type = other DNA
tgggataatctcctattcat

SEQ ID NO: 531 - DNA, 20 bp, synthetic construct
  source 1..20: mol_type = other DNA
aatgaataggagattatccc

SEQ ID NO: 532 - DNA, 20 bp, synthetic construct
  source 1..20: mol_type = other DNA
tatcccaggtgcctgcgtcc

SEQ ID NO: 533 - DNA, 20 bp, synthetic construct
  source 1..20: mol_type = other DNA
cagcctggacgcaggcacct

SEQ ID NO: 534 - DNA, 20 bp, synthetic construct
  source 1..20: mol_type = other DNA
ccaggtgcctgcgtccaggc

SEQ ID NO: 535 - DNA, 20 bp, synthetic construct
  source 1..20: mol_type = other DNA
ccagcctggacgcaggcacc

SEQ ID NO: 536 - DNA, 20 bp, synthetic construct
  source 1..20: mol_type = other DNA
cctgcgtccaggctggtgtc

SEQ ID NO: 537 - DNA, 20 bp, synthetic construct
  source 1..20: mol_type = other DNA
ccagacaccagcctggacgc

SEQ ID NO: 538 - DNA, 20 bp, synthetic construct
  source 1..20: mol_type = other DNA
ctgcgtccaggctggtgtct

SEQ ID NO: 539 - DNA, 20 bp, synthetic construct
  source 1..20: mol_type = other DNA
acagaacccagacaccagcc

SEQ ID NO: 540 - DNA, 20 bp, synthetic construct
  source 1..20: mol_type = other DNA
ctgtgccccttccccacacc

SEQ ID NO: 541 - DNA, 20 bp, synthetic construct
  source 1..20: mol_type = other DNA
ggacacctggtgtggggaag

SEQ ID NO: 542 - DNA, 20 bp, synthetic construct
  source 1..20: mol_type = other DNA
aggacacctggtgtggggaa

SEQ ID NO: 543 - DNA, 20 bp, synthetic construct
  source 1..20: mol_type = other DNA
caggacacctggtgtgggga

SEQ ID NO: 544 - DNA, 20 bp, synthetic construct
  source 1..20: mol_type = other DNA
tggacaggacacctggtgtg

SEQ ID NO: 545 - DNA, 20 bp, synthetic construct
  source 1..20: mol_type = other DNA
atggacaggacacctggtgt

SEQ ID NO: 546 - DNA, 20 bp, synthetic construct
  source 1..20: mol_type = other DNA
aatggacaggacacctggtg

SEQ ID NO: 547 - DNA, 20 bp, synthetic construct
  source 1..20: mol_type = other DNA
ctgagaatggacaggacacc

SEQ ID NO: 548 - DNA, 20 bp, synthetic construct
  source 1..20: mol_type = other DNA
caggtgtcctgtccattctc

SEQ ID NO: 549 - DNA, 20 bp, synthetic construct
  source 1..20: mol_type = other DNA
tgtcctgtccattctcaggc

SEQ ID NO: 550 - DNA, 20 bp, synthetic construct
  source 1..20: mol_type = other DNA
atgaggccaccctgaggtgc

SEQ ID NO: 551 - DNA, 20 bp, synthetic construct
  source 1..20: mol_type = other DNA
tgaggccaccctgaggtgct

SEQ ID NO: 552 - DNA, 20 bp, synthetic construct
  source 1..20: mol_type = other DNA
cagggcccagcacctcaggg

SEQ ID NO: 553 - DNA, 20 bp, synthetic construct
  source 1..20: mol_type = other DNA
caccctgaggtgctgggccc

SEQ ID NO: 554 - DNA, 20 bp, synthetic construct
  source 1..20: mol_type = other DNA
accctgaggtgctgggccct

SEQ ID NO: 555 - DNA, 20 bp, synthetic construct
  source 1..20: mol_type = other DNA
gcccagggcccagcacctca

SEQ ID NO: 556 - DNA, 20 bp, synthetic construct
  source 1..20: mol_type = other DNA
agcccagggcccagcacctc

SEQ ID NO: 557 - DNA, 20 bp, synthetic construct
  source 1..20: mol_type = other DNA
ggccctgggcttctaccctg

SEQ ID NO: 558 - DNA, 20 bp, synthetic construct
  source 1..20: mol_type = other DNA
ctccgcagggtagaagccca

SEQ ID NO: 559 - DNA, 20 bp, synthetic construct
  source 1..20: mol_type = other DNA
tctccgcagggtagaagccc

SEQ ID NO: 560 - DNA, 20 bp, synthetic construct
  source 1..20: mol_type = other DNA
aggtcagtgtgatctccgca

SEQ ID NO: 561 - DNA, 20 bp, synthetic construct
  source 1..20: mol_type = other DNA
caggtcagtgtgatctccgc

SEQ ID NO: 562 - DNA, 20 bp, synthetic construct
  source 1..20: mol_type = other DNA
ctgcggagatcacactgacc

SEQ ID NO: 563 - DNA, 20 bp, synthetic construct
  source 1..20: mol_type = other DNA
agatcacactgacctggcag

SEQ ID NO: 564 - DNA, 20 bp, synthetic construct
  source 1..20: mol_type = other DNA
gatcacactgacctggcagc

SEQ ID NO: 565 - DNA, 20 bp, synthetic construct
  source 1..20: mol_type = other DNA
acactgacctggcagcggga

SEQ ID NO: 566 - DNA, 20 bp, synthetic construct
  source 1..20: mol_type = other DNA
gacctggcagcgggatggcg

SEQ ID NO: 567 - DNA, 20 bp, synthetic construct
  source 1..20: mol_type = other DNA
gtcctcgccatcccgctgcc

SEQ ID NO: 568 - DNA, 20 bp, synthetic construct
  source 1..20: mol_type = other DNA
ggatggcgaggaccaaactc

SEQ ID NO: 569 - DNA, 20 bp, synthetic construct
  source 1..20: mol_type = other DNA
caagctcagtgtcctgagtt

SEQ ID NO: 570 - DNA, 20 bp, synthetic construct
  source 1..20: mol_type = other DNA
aactcaggacactgagcttg

SEQ ID NO: 571 - DNA, 20 bp, synthetic construct
  source 1..20: mol_type = other DNA
cttgtggagaccagaccagc

SEQ ID NO: 572 - DNA, 20 bp, synthetic construct
  source 1..20: mol_type = other DNA
ggttctatctcctgctggtc

SEQ ID NO: 573 - DNA, 20 bp, synthetic construct
  source 1..20: mol_type = other DNA
tggaaggttctatctcctgc

SEQ ID NO: 574 - DNA, 20 bp, synthetic construct
  source 1..20: mol_type = other DNA
cctgagatggggtaaggagg

SEQ ID NO: 575 - DNA, 20 bp, synthetic construct
  source 1..20: mol_type = other DNA
ccccctccttaccccatctc

SEQ ID NO: 576 - DNA, 20 bp, synthetic construct
  source 1..20: mol_type = other DNA
atggggtaaggagggggatg

SEQ ID NO: 577 - DNA, 20 bp, synthetic construct
  source 1..20: mol_type = other DNA
tggggtaaggagggggatga

SEQ ID NO: 578 - DNA, 20 bp, synthetic construct
  source 1..20: mol_type = other DNA
ggggtaaggagggggatgag

SEQ ID NO: 579 - DNA, 20 bp, synthetic construct
  source 1..20: mol_type = other DNA
gaggggtcatatctcttctc

SEQ ID NO: 580 - DNA, 20 bp, synthetic construct
  source 1..20: mol_type = other DNA
aggggtcatatctcttctca

SEQ ID NO: 581 - DNA, 20 bp, synthetic construct
  source 1..20: mol_type = other DNA
tatctcttctcagggaaagc

SEQ ID NO: 582 - DNA, 20 bp, synthetic construct
  source 1..20: mol_type = other DNA
agggaaagcaggagcccttc

SEQ ID NO: 583 - DNA, 20 bp, synthetic construct
  source 1..20: mol_type = other DNA
cccttctggagcccttcagc

SEQ ID NO: 584 - DNA, 20 bp, synthetic construct
  source 1..20: mol_type = other DNA
cctgctgaagggctccagaa

SEQ ID NO: 585 - DNA, 20 bp, synthetic construct
  source 1..20: mol_type = other DNA
ccttctggagcccttcagca

SEQ ID NO: 586 - DNA, 20 bp, synthetic construct
  source 1..20: mol_type = other DNA
ccctgctgaagggctccaga

SEQ ID NO: 587 - DNA, 20 bp, synthetic construct
  source 1..20: mol_type = other DNA
tggagcccttcagcagggtc

SEQ ID NO: 588 - DNA, 20 bp, synthetic construct
  source 1..20: mol_type = other DNA
ggagcccttcagcagggtca

SEQ ID NO: 589 - DNA, 20 bp, synthetic construct
  source 1..20: mol_type = other DNA
ggggccctgaccctgctgaa

SEQ ID NO: 590 - DNA, 20 bp, synthetic construct
  source 1..20: mol_type = other DNA
aggggccctgaccctgctga

SEQ ID NO: 591 - DNA, 20 bp, synthetic construct
  source 1..20: mol_type = other DNA
ggaaaggaggggaagacgag

SEQ ID NO: 592 - DNA, 20 bp, synthetic construct
  source 1..20: mol_type = other DNA
gggaaaggaggggaagacga

SEQ ID NO: 593 - DNA, 20 bp, synthetic construct
  source 1..20: mol_type = other DNA
tgggaaaggaggggaagacg

SEQ ID NO: 594 - DNA, 20 bp, synthetic construct
  source 1..20: mol_type = other DNA
agatggctctgggaaaggag

SEQ ID NO: 595 - DNA, 20 bp, synthetic construct
  source 1..20: mol_type = other DNA
aagatggctctgggaaagga

SEQ ID NO: 596 - DNA, 20 bp, synthetic construct
  source 1..20: mol_type = other DNA
gaagatggctctgggaaagg

SEQ ID NO: 597 - DNA, 20 bp, synthetic construct
  source 1..20: mol_type = other DNA
tgggaagatggctctgggaa

SEQ ID NO: 598 - DNA, 20 bp, synthetic construct
  source 1..20: mol_type = other DNA
tggactgggaagatggctct

SEQ ID NO: 599 - DNA, 20 bp, synthetic construct
  source 1..20: mol_type = other DNA
gtggactgggaagatggctc

SEQ ID NO: 600 - DNA, 20 bp, synthetic construct
  source 1..20: mol_type = other DNA
gggatggtggactgggaaga

SEQ ID NO: 601 - DNA, 20 bp, synthetic construct
  source 1..20: mol_type = other DNA
cacgatggggatggtggact

SEQ ID NO: 602 - DNA, 20 bp, synthetic construct
  source 1..20: mol_type = other DNA
tagggaaggggtgaggggtg

SEQ ID NO: 603 - DNA, 20 bp, synthetic construct
  source 1..20: mol_type = other DNA
aaggggtgaggggtggggtc

SEQ ID NO: 604 - DNA, 20 bp, synthetic construct
  source 1..20: mol_type = other DNA
aggggtgaggggtggggtct

SEQ ID NO: 605 - DNA, 20 bp, synthetic construct
  source 1..20: mol_type = other DNA
tctgggttttcttgtcccac

SEQ ID NO: 606 - DNA, 20 bp, synthetic construct
  source 1..20: mol_type = other DNA
ctgggttttcttgtcccact

SEQ ID NO: 607 - DNA, 20 bp, synthetic construct
  source 1..20: mol_type = other DNA
tgggttttcttgtcccactg

SEQ ID NO: 608 - DNA, 20 bp, synthetic construct
  source 1..20: mol_type = other DNA
gggttttcttgtcccactgg

SEQ ID NO: 609 - DNA, 20 bp, synthetic construct
  source 1..20: mol_type = other DNA
tggggcttgaaacccccagt

SEQ ID NO: 610 - DNA, 20 bp, synthetic construct
  source 1..20: mol_type = other DNA
ctggggcttgaaacccccag

SEQ ID NO: 611 - DNA, 20 bp, synthetic construct
  source 1..20: mol_type = other DNA
cactgggggtttcaagcccc

SEQ ID NO: 612 - DNA, 20 bp, synthetic construct
  source 1..20: mol_type = other DNA
gcagggaacacttctacctg

SEQ ID NO: 613 - DNA, 20 bp, synthetic construct
  source 1..20: mol_type = other DNA
ggcagggaacacttctacct

SEQ ID NO: 614 - DNA, 20 bp, synthetic construct
  source 1..20: mol_type = other DNA
aggcagggaacacttctacc

SEQ ID NO: 615 - DNA, 20 bp, synthetic construct
  source 1..20: mol_type = other DNA
agtgttccctgcctcattac

SEQ ID NO: 616 - DNA, 20 bp, synthetic construct
  source 1..20: mol_type = other DNA
gtgttccctgcctcattact

SEQ ID NO: 617 - DNA, 20 bp, synthetic construct
  source 1..20: mol_type = other DNA
tgcttcccagtaatgaggca

SEQ ID NO: 618 - DNA, 20 bp, synthetic construct
  source 1..20: mol_type = other DNA
ctgcttcccagtaatgaggc

SEQ ID NO: 619 - DNA, 20 bp, synthetic construct
  source 1..20: mol_type = other DNA
gatgctgcttcccagtaatg

SEQ ID NO: 620 - DNA, 20 bp, synthetic construct
  source 1..20: mol_type = other DNA
ctgggaagcagcatccacac

SEQ ID NO: 621 - DNA, 20 bp, synthetic construct
  source 1..20: mol_type = other DNA
tgggaagcagcatccacaca

SEQ ID NO: 622 - DNA, 20 bp, synthetic construct
  source 1..20: mol_type = other DNA
gggaagcagcatccacacag

SEQ ID NO: 623 - DNA, 20 bp, synthetic construct
  source 1..20: mol_type = other DNA
aggctgcgttagcccctgtg

SEQ ID NO: 624 - DNA, 20 bp, synthetic construct
  source 1..20: mol_type = other DNA
acacaggggctaacgcagcc

SEQ ID NO: 625 - DNA, 20 bp, synthetic construct
  source 1..20: mol_type = other DNA
cacaggggctaacgcagcct

SEQ ID NO: 626 - DNA, 20 bp, synthetic construct
  source 1..20: mol_type = other DNA
gtgctggcacacagggtccc

SEQ ID NO: 627 - DNA, 20 bp, synthetic construct
  source 1..20: mol_type = other DNA
agagtaagtgctggcacaca

SEQ ID NO: 628 - DNA, 20 bp, synthetic construct
  source 1..20: mol_type = other DNA
aagagtaagtgctggcacac

SEQ ID NO: 629 - DNA, 20 bp, synthetic construct
  source 1..20: mol_type = other DNA
cgtgtttcctgatcctgcct

SEQ ID NO: 630 - DNA, 20 bp, synthetic construct
  source 1..20: mol_type = other DNA
gtgtttcctgatcctgcctt

SEQ ID NO: 631 - DNA, 20 bp, synthetic construct
  source 1..20: mol_type = other DNA
tacagacccaaggcaggatc

SEQ ID NO: 632 - DNA, 20 bp, synthetic construct
  source 1..20: mol_type = other DNA
tatgactacagacccaaggc

SEQ ID NO: 633 - DNA, 20 bp, synthetic construct
  source 1..20: mol_type = other DNA
gaagtatgactacagaccca

SEQ ID NO: 634 - DNA, 20 bp, synthetic construct
  source 1..20: mol_type = other DNA
tgggtctgtagtcatacttc

SEQ ID NO: 635 - DNA, 20 bp, synthetic construct
  source 1..20: mol_type = other DNA
atacttctggaaattccttt

SEQ ID NO: 636 - DNA, 20 bp, synthetic construct
  source 1..20: mol_type = other DNA
tacttctggaaattcctttt

SEQ ID NO: 637 - DNA, 20 bp, synthetic construct
  source 1..20: mol_type = other DNA
ttccttttgggtccaagacg

SEQ ID NO: 638 - DNA, 20 bp, synthetic construct
  source 1..20: mol_type = other DNA
ctcctcgtcttggacccaaa

SEQ ID NO: 639 - DNA, 20 bp, synthetic construct
  source 1..20: mol_type = other DNA
cttttgggtccaagacgagg

SEQ ID NO: 640 - DNA, 20 bp, synthetic construct
  source 1..20: mol_type = other DNA
ttagaggaacctcctcgtct

SEQ ID NO: 641 - DNA, 20 bp, synthetic construct
  source 1..20: mol_type = other DNA
gaggttcctctaagatctca

SEQ ID NO: 642 - DNA, 20 bp, synthetic construct
  source 1..20: mol_type = other DNA
gcagggccatgagatcttag

SEQ ID NO: 643 - DNA, 20 bp, synthetic construct
  source 1..20: mol_type = other DNA
gaggggactgggaggaagca

SEQ ID NO: 644 - DNA, 20 bp, synthetic construct
  source 1..20: mol_type = other DNA
tgaggggactgggaggaagc

SEQ ID NO: 645 - DNA, 20 bp, synthetic construct
  source 1..20: mol_type = other DNA
cttcctcccagtcccctcac

SEQ ID NO: 646 - DNA, 20 bp, synthetic construct
  source 1..20: mol_type = other DNA
tgtcctgtgaggggactggg

SEQ ID NO: 647 - DNA, 20 bp, synthetic construct
  source 1..20: mol_type = other DNA
aaatgtcctgtgaggggact

SEQ ID NO: 648 - DNA, 20 bp, synthetic construct
  source 1..20: mol_type = other DNA
aaaatgtcctgtgaggggac

SEQ ID NO: 649 - DNA, 20 bp, synthetic construct
  source 1..20: mol_type = other DNA
ggaagaaaatgtcctgtgag

SEQ ID NO: 650 - DNA, 20 bp, synthetic construct
  source 1..20: mol_type = other DNA
gggaagaaaatgtcctgtga

SEQ ID NO: 651 - DNA, 20 bp, synthetic construct
  source 1..20: mol_type = other DNA
tgggaagaaaatgtcctgtg

SEQ ID NO: 652 - DNA, 20 bp, synthetic construct
  source 1..20: mol_type = other DNA
acaggacattttcttcccac

SEQ ID NO: 653 - DNA, 20 bp, synthetic construct
  source 1..20: mol_type = other DNA
ggacattttcttcccacagg

SEQ ID NO: 654 - DNA, 20 bp, synthetic construct
  source 1..20: mol_type = other DNA
gtcagagcccgcaggagacg

SEQ ID NO: 655 - DNA, 20 bp, synthetic construct
  source 1..20: mol_type = other DNA
gtctcctgcgggctctgacc

SEQ ID NO: 656 - DNA, 20 bp, synthetic construct
  source 1..20: mol_type = other DNA
aggacctggtcagagcccgc

SEQ ID NO: 657 - DNA, 20 bp, synthetic construct
  source 1..20: mol_type = other DNA
agtagaacaaaaacaggacc

SEQ ID NO: 658 - DNA, 20 bp, synthetic construct
  source 1..20: mol_type = other DNA
ggctggagtagaacaaaaac

SEQ ID NO: 659 - DNA, 20 bp, synthetic construct
  source 1..20: mol_type = other DNA
tccagccagcgacagtgccc

SEQ ID NO: 660 - DNA, 20 bp, synthetic construct
  source 1..20: mol_type = other DNA
ccagccagcgacagtgccca

SEQ ID NO: 661 - DNA, 20 bp, synthetic construct
  source 1..20: mol_type = other DNA
ccctgggcactgtcgctggc

SEQ ID NO: 662 - DNA, 20 bp, synthetic construct
  source 1..20: mol_type = other DNA
agagccctgggcactgtcgc

SEQ ID NO: 663 - DNA, 20 bp, synthetic construct
  source 1..20: mol_type = other DNA
gagagacacatcagagccct

SEQ ID NO: 664 - DNA, 20 bp, synthetic construct
  source 1..20: mol_type = other DNA
tgagagacacatcagagccc

SEQ ID NO: 665 - DNA, 20 bp, synthetic construct
  source 1..20: mol_type = other DNA
gtgtctctcacagcttgaaa

SEQ ID NO: 666 - DNA, 20 bp, synthetic construct
  source 1..20: mol_type = other DNA
gcttgaaaaggtgagattct

SEQ ID NO: 667 - DNA, 20 bp, synthetic construct
  source 1..20: mol_type = other DNA
cttgaaaaggtgagattctt

SEQ ID NO: 668 - DNA, 20 bp, synthetic construct
  source 1..20: mol_type = other DNA
ttgaaaaggtgagattcttg

SEQ ID NO: 669 - DNA, 20 bp, synthetic construct
  source 1..20: mol_type = other DNA
gagattcttggggtctagag

SEQ ID NO: 670 - DNA, 20 bp, synthetic construct
  source 1..20: mol_type = other DNA
agattcttggggtctagagt

SEQ ID NO: 671 - DNA, 20 bp, synthetic construct
  source 1..20: mol_type = other DNA
ttcttggggtctagagtggg

SEQ ID NO: 672 - DNA, 20 bp, synthetic construct
  source 1..20: mol_type = other DNA
tcttggggtctagagtgggc

SEQ ID NO: 673 - DNA, 20 bp, synthetic construct
  source 1..20: mol_type = other DNA
cttggggtctagagtgggcg

SEQ ID NO: 674 - DNA, 20 bp, synthetic construct
  source 1..20: mol_type = other DNA
ttggggtctagagtgggcgg

SEQ ID NO: 675 - DNA, 20 bp, synthetic construct
  source 1..20: mol_type = other DNA
tggggtctagagtgggcggg

SEQ ID NO: 676 - DNA, 20 bp, synthetic construct
  source 1..20: mol_type = other DNA
ggggtctagagtgggcgggg

SEQ ID NO: 677 - DNA, 20 bp, synthetic construct
  source 1..20: mol_type = other DNA
gggtctagagtgggcggggg

SEQ ID NO: 678 - DNA, 20 bp, synthetic construct
  source 1..20: mol_type = other DNA
tctagagtgggcgggggggg

SEQ ID NO: 679 - DNA, 20 bp, synthetic construct
  source 1..20: mol_type = other DNA
gtcactcaccggcctcgctc

SEQ ID NO: 680 - DNA, 20 bp, synthetic construct
  source 1..20: mol_type = other DNA
gcgaggccggtgagtgaccc

SEQ ID NO: 681 - DNA, 20 bp, synthetic construct
  source 1..20: mol_type = other DNA
gccggtgagtgaccccggcc

SEQ ID NO: 682 - DNA, 20 bp, synthetic construct
  source 1..20: mol_type = other DNA
ccggtgagtgaccccggccc

SEQ ID NO: 683 - DNA, 20 bp, synthetic construct
  source 1..20: mol_type = other DNA
cccgggccggggtcactcac

SEQ ID NO: 684 - DNA, 20 bp, synthetic construct
  source 1..20: mol_type = other DNA
cggtgagtgaccccggcccg

SEQ ID NO: 685 - DNA, 20 bp, synthetic construct
  source 1..20: mol_type = other DNA
gtgaccccggcccggggcgc

SEQ ID NO: 686 - DNA, 20 bp, synthetic construct
  source 1..20: mol_type = other DNA
atctcggacccggagactcg

SEQ ID NO: 687 - DNA, 20 bp, synthetic construct
  source 1..20: mol_type = other DNA
gatctcggacccggagactc

SEQ ID NO: 688 - DNA, 20 bp, synthetic construct
  source 1..20: mol_type = other DNA
ggatctcggacccggagact

SEQ ID NO: 689 - DNA, 20 bp, synthetic construct
  source 1..20: mol_type = other DNA
agggaggcggatctcggacc

SEQ ID NO: 690 - DNA, 20 bp, synthetic construct
  source 1..20: mol_type = other DNA
gtccgagatccgcctccctg

SEQ ID NO: 691 - DNA, 20 bp, synthetic construct
  source 1..20: mol_type = other DNA
ggcctcagggaggcggatct

SEQ ID NO: 692 - DNA, 20 bp, synthetic construct
  source 1..20: mol_type = other DNA
gatccgcctccctgaggccg

SEQ ID NO: 693 - DNA, 20 bp, synthetic construct
  source 1..20: mol_type = other DNA
atccgcctccctgaggccgc

SEQ ID NO: 694 - DNA, 20 bp, synthetic construct
  source 1..20: mol_type = other DNA
gtcccgcggcctcagggagg

SEQ ID NO: 695 - DNA, 20 bp, synthetic construct
  source 1..20: mol_type = other DNA
cgggtcccgcggcctcaggg

SEQ ID NO: 696 - DNA, 20 bp, synthetic construct
  source 1..20: mol_type = other DNA
gggcgggtcccgcggcctca

SEQ ID NO: 697 - DNA, 20 bp, synthetic construct
  source 1..20: mol_type = other DNA
tgggcgggtcccgcggcctc

SEQ ID NO: 698 - DNA, 20 bp, synthetic construct
  source 1..20: mol_type = other DNA
gagggtctgggcgggtcccg

SEQ ID NO: 699 - DNA, 20 bp, synthetic construct
  source 1..20: mol_type = other DNA
gacccgcccagaccctcgac

SEQ ID NO: 700 - DNA, 20 bp, synthetic construct
  source 1..20: mol_type = other DNA
cgccggtcgagggtctgggc

SEQ ID NO: 701 - DNA, 20 bp, synthetic construct
  source 1..20: mol_type = other DNA
tcgccggtcgagggtctggg

SEQ ID NO: 702 - DNA, 20 bp, synthetic construct
  source 1..20: mol_type = other DNA
ctctcgccggtcgagggtct

SEQ ID NO: 703 - DNA, 20 bp, synthetic construct
  source 1..20: mol_type = other DNA
gctctcgccggtcgagggtc

SEQ ID NO: 704 - DNA, 20 bp, synthetic construct
  source 1..20: mol_type = other DNA
ctggggctctcgccggtcga

SEQ ID NO: 705 - DNA, 20 bp, synthetic construct
  source 1..20: mol_type = other DNA
agttgaggccaaaatccccg

SEQ ID NO: 706 - DNA, 20 bp, synthetic construct
  source 1..20: mol_type = other DNA
gttgaggccaaaatccccgc

SEQ ID NO: 707 - DNA, 20 bp, synthetic construct
  source 1..20: mol_type = other DNA
aggccaaaatccccgcgggt

SEQ ID NO: 708 - DNA, 20 bp, synthetic construct
  source 1..20: mol_type = other DNA
cgaccaacccgcggggattt

SEQ ID NO: 709 - DNA, 20 bp, synthetic construct
  source 1..20: mol_type = other DNA
caaaatccccgcgggttggt

SEQ ID NO: 710 - DNA, 20 bp, synthetic construct
  source 1..20: mol_type = other DNA
aaaatccccgcgggttggtc

SEQ ID NO: 711 - DNA, 20 bp, synthetic construct
  source 1..20: mol_type = other DNA
aaatccccgcgggttggtcg

SEQ ID NO: 712 - DNA, 20 bp, synthetic construct
  source 1..20: mol_type = other DNA
tccccgcgggttggtcgggg

SEQ ID NO: 713 - DNA, 20 bp, synthetic construct
  source 1..20: mol_type = other DNA
ccccgcgggttggtcggggc

SEQ ID NO: 714 - DNA, 20 bp, synthetic construct
  source 1..20: mol_type = other DNA
cccgccccgaccaacccgcg

SEQ ID NO: 715 - DNA, 20 bp, synthetic construct
  source 1..20: mol_type = other DNA
acggggctgaccgcggggcc

SEQ ID NO: 716 - DNA, 20 bp, synthetic construct
  source 1..20: mol_type = other DNA
cggggctgaccgcggggccg

SEQ ID NO: 717 - DNA, 20 bp, synthetic construct
  source 1..20: mol_type = other DNA
ctgaccgcggggccggggcc

SEQ ID NO: 718 - DNA, 20 bp, synthetic construct
  source 1..20: mol_type = other DNA
tgaccgcggggccggggcca

SEQ ID NO: 719 - DNA, 20 bp, synthetic construct
  source 1..20: mol_type = other DNA
agaccctggccccggccccg

SEQ ID NO: 720 - DNA, 20 bp, synthetic construct
  source 1..20: mol_type = other DNA
atgatgtgagaccctggccc

SEQ ID NO: 721 - DNA, 20 bp, synthetic construct
  source 1..20: mol_type = other DNA
ctctggatgatgtgagaccc

SEQ ID NO: 722 - DNA, 20 bp, synthetic construct
  source 1..20: mol_type = other DNA
cagggtctcacatcatccag

SEQ ID NO: 723 - DNA, 20 bp, synthetic construct
  source 1..20: mol_type = other DNA
cacatcatccagaggatgta

SEQ ID NO: 724 - DNA, 20 bp, synthetic construct
  source 1..20: mol_type = other DNA
ggtcgcagccatacatcctc

SEQ ID NO: 725 - DNA, 20 bp, synthetic construct
  source 1..20: mol_type = other DNA
gaggatgtatggctgcgacc

SEQ ID NO: 726 - DNA, 20 bp, synthetic construct
  source 1..20: mol_type = other DNA
aggatgtatggctgcgacct

SEQ ID NO: 727 - DNA, 20 bp, synthetic construct
  source 1..20: mol_type = other DNA
ggatgtatggctgcgacctg

SEQ ID NO: 728 - DNA, 20 bp, synthetic construct
  source 1..20: mol_type = other DNA
ggctgcgacctggggcccga

SEQ ID NO: 729 - DNA, 20 bp, synthetic construct
  source 1..20: mol_type = other DNA
gctgcgacctggggcccgac

SEQ ID NO: 730 - DNA, 20 bp, synthetic construct
  source 1..20: mol_type = other DNA
ggaggcgcccgtcgggcccc

SEQ ID NO: 731 - DNA, 20 bp, synthetic construct
  source 1..20: mol_type = other DNA
cccgacgggcgcctcctccg

SEQ ID NO: 732 - DNA, 20 bp, synthetic construct
  source 1..20: mol_type = other DNA
cctgagctcctggaccgcgg

SEQ ID NO: 733 - DNA, 20 bp, synthetic construct
  source 1..20: mol_type = other DNA
ccgccgcggtccaggagctc

SEQ ID NO: 734 - DNA, 20 bp, synthetic construct
  source 1..20: mol_type = other DNA
cgcggtgtccgccgcggtcc

SEQ ID NO: 735 - DNA, 20 bp, synthetic construct
  source 1..20: mol_type = other DNA
ctggaccgcggcggacaccg

SEQ ID NO: 736 - DNA, 20 bp, synthetic construct
  source 1..20: mol_type = other DNA
ctgagccgcggtgtccgccg

SEQ ID NO: 737 - DNA, 20 bp, synthetic construct
  source 1..20: mol_type = other DNA
gcgctgggtgatctgagccg

SEQ ID NO: 738 - DNA, 20 bp, synthetic construct
  source 1..20: mol_type = other DNA
ctcagatcacccagcgcaag

SEQ ID NO: 739 - DNA, 20 bp, synthetic construct
  source 1..20: mol_type = other DNA
tcagatcacccagcgcaagt

SEQ ID NO: 740 - DNA, 20 bp, synthetic construct
  source 1..20: mol_type = other DNA
gatcacccagcgcaagtggg

SEQ ID NO: 741 - DNA, 20 bp, synthetic construct
  source 1..20: mol_type = other DNA
cacccagcgcaagtgggagg

SEQ ID NO: 742 - DNA, 20 bp, synthetic construct
  source 1..20: mol_type = other DNA
ggccgcctcccacttgcgct

SEQ ID NO: 743 - DNA, 20 bp, synthetic construct
  source 1..20: mol_type = other DNA
gggccgcctcccacttgcgc

SEQ ID NO: 744 - DNA, 20 bp, synthetic construct
  source 1..20: mol_type = other DNA
caagtgggaggcggcccgtg

SEQ ID NO: 745 - DNA, 20 bp, synthetic construct
  source 1..20: mol_type = other DNA
gtgggaggcggcccgtgtgg

SEQ ID NO: 746 - DNA, 20 bp, synthetic construct
  source 1..20: mol_type = other DNA
tctcagctgctccgccacac

SEQ ID NO: 747 - DNA, 20 bp, synthetic construct
  source 1..20: mol_type = other DNA
ctctcagctgctccgccaca

SEQ ID NO: 748 - DNA, 20 bp, synthetic construct
  source 1..20: mol_type = other DNA
cgcgcgggtaccaggggcag

SEQ ID NO: 749 - DNA, 20 bp, synthetic construct
  source 1..20: mol_type = other DNA
gcgcgggtaccaggggcagt

SEQ ID NO: 750 - DNA, 20 bp, synthetic construct
  source 1..20: mol_type = other DNA
cgcgggtaccaggggcagtg

SEQ ID NO: 751 - DNA, 20 bp, synthetic construct
  source 1..20: mol_type = other DNA
ggaaggctccccactgcccc

SEQ ID NO: 752 - DNA, 20 bp, synthetic construct
  source 1..20: mol_type = other DNA
gagccttccccatctcctat

SEQ ID NO: 753 - DNA, 20 bp, synthetic construct
  source 1..20: mol_type = other DNA
cgacctataggagatgggga

SEQ ID NO: 754 - DNA, 20 bp, synthetic construct
  source 1..20: mol_type = other DNA
ccccatctcctataggtcgc

SEQ ID NO: 755 - DNA, 20 bp, synthetic construct
  source 1..20: mol_type = other DNA
ccggcgacctataggagatg

SEQ ID NO: 756 - DNA, 20 bp, synthetic construct
  source 1..20: mol_type = other DNA
cccatctcctataggtcgcc

SEQ ID NO: 757 - DNA, 20 bp, synthetic construct
  source 1..20: mol_type = other DNA
cccggcgacctataggagat

SEQ ID NO: 758 - DNA, 20 bp, synthetic construct
  source 1..20: mol_type = other DNA
ccatctcctataggtcgccg

SEQ ID NO: 759 - DNA, 20 bp, synthetic construct
  source 1..20: mol_type = other DNA
ccccggcgacctataggaga

SEQ ID NO: 760 - DNA, 20 bp, synthetic construct
  source 1..20: mol_type = other DNA
ctcctataggtcgccgggga

SEQ ID NO: 761 - DNA, 20 bp, synthetic construct
  source 1..20: mol_type = other DNA
ggccatccccggcgacctat

SEQ ID NO: 762 - DNA, 20 bp, synthetic construct
  source 1..20: mol_type = other DNA
ttctcgtgggaggccatccc

SEQ ID NO: 763 - DNA, 20 bp, synthetic construct
  source 1..20: mol_type = other DNA
ggatggcctcccacgagaag

SEQ ID NO: 764 - DNA, 20 bp, synthetic construct
  source 1..20: mol_type = other DNA
tggcctcccacgagaagagg

SEQ ID NO: 765 - DNA, 20 bp, synthetic construct
  source 1..20: mol_type = other DNA
tttcctcctcttctcgtggg

SEQ ID NO: 766 - DNA, 20 bp, synthetic construct
  source 1..20: mol_type = other DNA
cattttcctcctcttctcgt

SEQ ID NO: 767 - DNA, 20 bp, synthetic construct
  source 1..20: mol_type = other DNA
cagtccctagaatactgatc

SEQ ID NO: 768 - DNA, 20 bp, synthetic construct
  source 1..20: mol_type = other DNA
agtccctagaatactgatca

SEQ ID NO: 769 - DNA, 20 bp, synthetic construct
  source 1..20: mol_type = other DNA
gtccctagaatactgatcag

SEQ ID NO: 770 - DNA, 20 bp, synthetic construct
  source 1..20: mol_type = other DNA
gacccctgatcagtattcta

SEQ ID NO: 771 - DNA, 20 bp, synthetic construct
  source 1..20: mol_type = other DNA
ggacccctgatcagtattct

SEQ ID NO: 772 - DNA, 20 bp, synthetic construct
  source 1..20: mol_type = other DNA
ggctgctgcaggggtcaaag

SEQ ID NO: 773 - DNA, 20 bp, synthetic construct
  source 1..20: mol_type = other DNA
aggctgctgcaggggtcaaa

SEQ ID NO: 774 - DNA, 20 bp, synthetic construct
  source 1..20: mol_type = other DNA
aaggctgctgcaggggtcaa

SEQ ID NO: 775 - DNA, 20 bp, synthetic construct
  source 1..20: mol_type = other DNA
tttgacccctgcagcagcct

SEQ ID NO: 776 - DNA, 20 bp, synthetic construct
  source 1..20: mol_type = other DNA
ttgacccctgcagcagcctt

SEQ ID NO: 777 - DNA, 20 bp, synthetic construct
  source 1..20: mol_type = other DNA
ggttcccaaggctgctgcag

SEQ ID NO: 778 - DNA, 20 bp, synthetic construct
  source 1..20: mol_type = other DNA
cggttcccaaggctgctgca

SEQ ID NO: 779 - DNA, 20 bp, synthetic construct
  source 1..20: mol_type = other DNA
acggttcccaaggctgctgc

SEQ ID NO: 780 - DNA, 20 bp, synthetic construct
  source 1..20: mol_type = other DNA
aggaaaagtcacggttccca

SEQ ID NO: 781 - DNA, 20 bp, synthetic construct
  source 1..20: mol_type = other DNA
gaaccgtgacttttcctctc

SEQ ID NO: 782 - DNA, 20 bp, synthetic construct
  source 1..20: mol_type = other DNA
aggcctgagaggaaaagtca

SEQ ID NO: 783 - DNA, 20 bp, synthetic construct
  source 1..20: mol_type = other DNA
ggcagagaacaaggcctgag

SEQ ID NO: 784 - DNA, 20 bp, synthetic construct
  source 1..20: mol_type = other DNA
tgagtgtgaggcagagaaca

SEQ ID NO: 785 - DNA, 20 bp, synthetic construct
  source 1..20: mol_type = other DNA
tgcctcacactcagtgtgtt

SEQ ID NO: 786 - DNA, 20 bp, synthetic construct
  source 1..20: mol_type = other DNA
gcctcacactcagtgtgttt

SEQ ID NO: 787 - DNA, 20 bp, synthetic construct
  source 1..20: mol_type = other DNA
cctcacactcagtgtgtttg

SEQ ID NO: 788 - DNA, 20 bp, synthetic construct
  source 1..20: mol_type = other DNA
ccccaaacacactgagtgtg

SEQ ID NO: 789 - DNA, 20 bp, synthetic construct
  source 1..20: mol_type = other DNA
gtaaagtgactcagaagtgc

SEQ ID NO: 790 - DNA, 20 bp, synthetic construct
  source 1..20: mol_type = other DNA
actttacctccactcagatc

SEQ ID NO: 791 - DNA, 20 bp, synthetic construct
  source 1..20: mol_type = other DNA
tctgctcctgatctgagtgg

SEQ ID NO: 792 - DNA, 20 bp, synthetic construct
  source 1..20: mol_type = other DNA
tgaccagcctgagaatggac

SEQ ID NO: 793 - DNA, 20 bp, synthetic construct
  source 1..20: mol_type = other DNA
ccattctcaggctggtcaca

SEQ ID NO: 794 - DNA, 20 bp, synthetic construct
  source 1..20: mol_type = other DNA
ccatgtgaccagcctgagaa

SEQ ID NO: 795 - DNA, 20 bp, synthetic construct
  source 1..20: mol_type = other DNA
cattctcaggctggtcacat

SEQ ID NO: 796 - DNA, 20 bp, synthetic construct
  source 1..20: mol_type = other DNA
tctcaggctggtcacatggg

SEQ ID NO: 797 - DNA, 20 bp, synthetic construct
  source 1..20: mol_type = other DNA
ctggtcacatgggtggtcct

SEQ ID NO: 798 - DNA, 20 bp, synthetic construct
  source 1..20: mol_type = other DNA
tggtcacatgggtggtccta

SEQ ID NO: 799 - DNA, 20 bp, synthetic construct
  source 1..20: mol_type = other DNA
catctctcatgggacaccct

SEQ ID NO: 800 - DNA, 20 bp, synthetic construct
  source 1..20: mol_type = other DNA
aggcgctttgcatctctcat

SEQ ID NO: 801 - DNA, 20 bp, synthetic construct
  source 1..20: mol_type = other DNA
caggcgctttgcatctctca

SEQ ID NO: 802 - DNA, 20 bp, synthetic construct
  source 1..20: mol_type = other DNA
tgggaagagtcagaaaattc

SEQ ID NO: 803 - DNA, 20 bp, synthetic construct
  source 1..20: mol_type = other DNA
gtgtctttggggggtctgat

SEQ ID NO: 804 - DNA, 20 bp, synthetic construct
  source 1..20: mol_type = other DNA
tgtgtctttggggggtctga

SEQ ID NO: 805 - DNA, 20 bp, synthetic construct
  source 1..20: mol_type = other DNA
gggtcacgtgtgtctttggg

SEQ ID NO: 806 - DNA, 20 bp, synthetic construct
  source 1..20: mol_type = other DNA
tgggtcacgtgtgtctttgg

SEQ ID NO: 807 - DNA, 20 bp, synthetic construct
  source 1..20: mol_type = other DNA
gtgggtcacgtgtgtctttg

SEQ ID NO: 808 - DNA, 20 bp, synthetic construct
  source 1..20: mol_type = other DNA
ggtgggtcacgtgtgtcttt

SEQ ID NO: 809 - DNA, 20 bp, synthetic construct
  source 1..20: mol_type = other DNA
tggtgggtcacgtgtgtctt

SEQ ID NO: 810 - DNA, 20 bp, synthetic construct
  source 1..20: mol_type = other DNA
atggtcagagacggggtggt

SEQ ID NO: 811 - DNA, 20 bp, synthetic construct
  source 1..20: mol_type = other DNA
catggtcagagacggggtgg

SEQ ID NO: 812 - DNA, 20 bp, synthetic construct
  source 1..20: mol_type = other DNA
ccaccccgtctctgaccatg

SEQ ID NO: 813 - DNA, 20 bp, synthetic construct
  source 1..20: mol_type = other DNA
cctcatggtcagagacgggg

SEQ ID NO: 814 - DNA, 20 bp, synthetic construct
  source 1..20: mol_type = other DNA
tggcctcatggtcagagacg

SEQ ID NO: 815 - DNA, 20 bp, synthetic construct
  source 1..20: mol_type = other DNA
gtggcctcatggtcagagac

SEQ ID NO: 816 - DNA, 20 bp, synthetic construct
  source 1..20: mol_type = other DNA
ggtggcctcatggtcagaga

SEQ ID NO: 817 - DNA, 20 bp, synthetic construct
  source 1..20: mol_type = other DNA
ctgaccatgaggccaccctg

SEQ ID NO: 818 - DNA, 20 bp, synthetic construct
  source 1..20: mol_type = other DNA
agcacctcagggtggcctca

SEQ ID NO: 819 - DNA, 20 bp, synthetic construct
  source 1..20: mol_type = other DNA
gagatagaaccttccagaag

SEQ ID NO: 820 - DNA, 20 bp, synthetic construct
  source 1..20: mol_type = other DNA
agatagaaccttccagaagt

SEQ ID NO: 821 - DNA, 20 bp, synthetic construct
  source 1..20: mol_type = other DNA
cacagctgcccacttctgga

SEQ ID NO: 822 - DNA, 20 bp, synthetic construct
  source 1..20: mol_type = other DNA
cttccagaagtgggcagctg

SEQ ID NO: 823 - DNA, 20 bp, synthetic construct
  source 1..20: mol_type = other DNA
ccagaagtgggcagctgtgg

SEQ ID NO: 824 - DNA, 20 bp, synthetic construct
  source 1..20: mol_type = other DNA
ccaccacagctgcccacttc

SEQ ID NO: 825 - DNA, 20 bp, synthetic construct
  source 1..20: mol_type = other DNA
gcagctgtggtggtgccttc

SEQ ID NO: 826 - DNA, 20 bp, synthetic construct
  source 1..20: mol_type = other DNA
tatctctgctcttctccaga

SEQ ID NO: 827 - DNA, 20 bp, synthetic construct
  source 1..20: mol_type = other DNA
cacatgccatgtacagcatg

SEQ ID NO: 828 - DNA, 20 bp, synthetic construct
  source 1..20: mol_type = other DNA
acatgccatgtacagcatga

SEQ ID NO: 829 - DNA, 20 bp, synthetic construct
  source 1..20: mol_type = other DNA
catgccatgtacagcatgag

SEQ ID NO: 830 - DNA, 20 bp, synthetic construct
  source 1..20: mol_type = other DNA
gcagcccctcatgctgtaca

SEQ ID NO: 831 - DNA, 20 bp, synthetic construct
  source 1..20: mol_type = other DNA
catctcagggtgaggggctt

SEQ ID NO: 832 - DNA, 20 bp, synthetic construct
  source 1..20: mol_type = other DNA
cgaagcccctcaccctgaga

SEQ ID NO: 833 - DNA, 20 bp, synthetic construct
  source 1..20: mol_type = other DNA
gaagcccctcaccctgagat

SEQ ID NO: 834 - DNA, 20 bp, synthetic construct
  source 1..20: mol_type = other DNA
aagcccctcaccctgagatg

SEQ ID NO: 835 - DNA, 20 bp, synthetic construct
  source 1..20: mol_type = other DNA
ttaccccatctcagggtgag

SEQ ID NO: 836 - DNA, 20 bp, synthetic construct
  source 1..20: mol_type = other DNA
cttaccccatctcagggtga

SEQ ID NO: 837 - DNA, 20 bp, synthetic construct
  source 1..20: mol_type = other DNA
cctcaccctgagatggggta

SEQ ID NO: 838 - DNA, 20 bp, synthetic construct
  source 1..20: mol_type = other DNA
ccttaccccatctcagggtg

SEQ ID NO: 839 - DNA, 20 bp, synthetic construct
  source 1..20: mol_type = other DNA
caccctgagatggggtaagg

SEQ ID NO: 840 - DNA, 20 bp, synthetic construct
  source 1..20: mol_type = other DNA
accctgagatggggtaagga

SEQ ID NO: 841 - DNA, 20 bp, synthetic construct
  source 1..20: mol_type = other DNA
ccctgagatggggtaaggag

SEQ ID NO: 842 - DNA, 20 bp, synthetic construct
  source 1..20: mol_type = other DNA
cccctccttaccccatctca

SEQ ID NO: 843 - DNA, 20 bp, synthetic construct
  source 1..20: mol_type = other DNA
ccagtccaccatccccatcg

SEQ ID NO: 844 - DNA, 20 bp, synthetic construct
  source 1..20: mol_type = other DNA
ccacgatggggatggtggac

SEQ ID NO: 845 - DNA, 20 bp, synthetic construct
  source 1..20: mol_type = other DNA
cagtccaccatccccatcgt

SEQ ID NO: 846 - DNA, 20 bp, synthetic construct
  source 1..20: mol_type = other DNA
aatgcccacgatggggatgg

SEQ ID NO: 847 - DNA, 20 bp, synthetic construct
  source 1..20: mol_type = other DNA
aacaatgcccacgatgggga

SEQ ID NO: 848 - DNA, 20 bp, synthetic construct
  source 1..20: mol_type = other DNA
cagcaacaatgcccacgatg

SEQ ID NO: 849 - DNA, 20 bp, synthetic construct
  source 1..20: mol_type = other DNA
cccatcgtgggcattgttgc

SEQ ID NO: 850 - DNA, 20 bp, synthetic construct
  source 1..20: mol_type = other DNA
ccagcaacaatgcccacgat

SEQ ID NO: 851 - DNA, 20 bp, synthetic construct
  source 1..20: mol_type = other DNA
gccagcaacaatgcccacga

SEQ ID NO: 852 - DNA, 20 bp, synthetic construct
  source 1..20: mol_type = other DNA
cgtgggcattgttgctggcc

SEQ ID NO: 853 - DNA, 20 bp, synthetic construct
  source 1..20: mol_type = other DNA
cctggctgtcctagcagttg

SEQ ID NO: 854 - DNA, 20 bp, synthetic construct
  source 1..20: mol_type = other DNA
ccacaactgctaggacagcc

SEQ ID NO: 855 - DNA, 20 bp, synthetic construct
  source 1..20: mol_type = other DNA
gtcctagcagttgtggtcat

SEQ ID NO: 856 - DNA, 20 bp, synthetic construct
  source 1..20: mol_type = other DNA
ctccgatgaccacaactgct

SEQ ID NO: 857 - DNA, 20 bp, synthetic construct
  source 1..20: mol_type = other DNA
agttgtggtcatcggagctg

SEQ ID NO: 858 - DNA, 20 bp, synthetic construct
  source 1..20: mol_type = other DNA
tggtcgctactgtgatgtgt

SEQ ID NO: 859 - DNA, 20 bp, synthetic construct
  source 1..20: mol_type = other DNA
tcgctactgtgatgtgtagg

SEQ ID NO: 860 - DNA, 20 bp, synthetic construct
  source 1..20: mol_type = other DNA
atgtgtaggaggaagagctc

SEQ ID NO: 861 - DNA, 20 bp, synthetic construct
  source 1..20: mol_type = other DNA
gtaggaggaagagctcaggt

SEQ ID NO: 862 - DNA, 20 bp, synthetic construct
  source 1..20: mol_type = other DNA
taggaggaagagctcaggta

SEQ ID NO: 863 - DNA, 20 bp, synthetic construct
  source 1..20: mol_type = other DNA
aggaagagctcaggtaggga

SEQ ID NO: 864 - DNA, 20 bp, synthetic construct
  source 1..20: mol_type = other DNA
ggaagagctcaggtagggaa

SEQ ID NO: 865 - DNA, 20 bp, synthetic construct
  source 1..20: mol_type = other DNA
gaagagctcaggtagggaag

SEQ ID NO: 866 - DNA, 20 bp, synthetic construct
  source 1..20: mol_type = other DNA
gctcaggtagggaaggggtg

SEQ ID NO: 867 - DNA, 20 bp, synthetic construct
  source 1..20: mol_type = other DNA
ctcaggtagggaaggggtga

SEQ ID NO: 868 - DNA, 20 bp, synthetic construct
  source 1..20: mol_type = other DNA
tcaggtagggaaggggtgag

SEQ ID NO: 869 - DNA, 20 bp, synthetic construct
  source 1..20: mol_type = other DNA
ggtagggaaggggtgagggg

SEQ ID NO: 870 - DNA, 20 bp, synthetic construct
  source 1..20: mol_type = other DNA
gtagggaaggggtgaggggt

SEQ ID NO: 871 - DNA, 20 bp, synthetic construct
  source 1..20: mol_type = other DNA
ctgcacaaaagagtaagtgc

SEQ ID NO: 872 - DNA, 20 bp, synthetic construct
  source 1..20: mol_type = other DNA
tgcagcacatgtgacaatga

SEQ ID NO: 873 - DNA, 20 bp, synthetic construct
  source 1..20: mol_type = other DNA
gcacatgtgacaatgaagga

SEQ ID NO: 874 - DNA, 20 bp, synthetic construct
  source 1..20: mol_type = other DNA
ggacggatgtatcaccttga

SEQ ID NO: 875 - DNA, 20 bp, synthetic construct
  source 1..20: mol_type = other DNA
atgtatcaccttgatggttg

SEQ ID NO: 876 - DNA, 20 bp, synthetic construct
  source 1..20: mol_type = other DNA
caccttgatggttgtggtgt

SEQ ID NO: 877 - DNA, 20 bp, synthetic construct
  source 1..20: mol_type = other DNA
accttgatggttgtggtgtt

SEQ ID NO: 878 - DNA, 20 bp, synthetic construct
  source 1..20: mol_type = other DNA
ccttgatggttgtggtgttg

SEQ ID NO: 879 - DNA, 20 bp, synthetic construct
  source 1..20: mol_type = other DNA
ccccaacaccacaaccatca

SEQ ID NO: 880 - DNA, 20 bp, synthetic construct
  source 1..20: mol_type = other DNA
gactcatgaatgctgaaatc

SEQ ID NO: 881 - DNA, 20 bp, synthetic construct
  source 1..20: mol_type = other DNA
gatttcagcattcatgagtc

SEQ ID NO: 882 - DNA, 20 bp, synthetic construct
  source 1..20: mol_type = other DNA
atttcagcattcatgagtca

SEQ ID NO: 883 - DNA, 20 bp, synthetic construct
  source 1..20: mol_type = other DNA
tttcagcattcatgagtcag

SEQ ID NO: 884 - DNA, 20 bp, synthetic construct
  source 1..20: mol_type = other DNA
agcattcatgagtcagggga

SEQ ID NO: 885 - DNA, 20 bp, synthetic construct
  source 1..20: mol_type = other DNA
tcaggggaaggtccctgcta

SEQ ID NO: 886 - DNA, 20 bp, synthetic construct
  source 1..20: mol_type = other DNA
ccctgctaaggacagacctt

SEQ ID NO: 887 - DNA, 20 bp, synthetic construct
  source 1..20: mol_type = other DNA
cctaaggtctgtccttagca

SEQ ID NO: 888 - DNA, 20 bp, synthetic construct
  source 1..20: mol_type = other DNA
tcctaaggtctgtccttagc

SEQ ID NO: 889 - DNA, 20 bp, synthetic construct
  source 1..20: mol_type = other DNA
tgctaaggacagaccttagg

SEQ ID NO: 890 - DNA, 20 bp, synthetic construct
  source 1..20: mol_type = other DNA
gctaaggacagaccttagga

SEQ ID NO: 891 - DNA, 20 bp, synthetic construct
  source 1..20: mol_type = other DNA
acagaccttaggagggcagt

SEQ ID NO: 892 - DNA, 20 bp, synthetic construct
  source 1..20: mol_type = other DNA
ctggaccaactgccctccta

SEQ ID NO: 893 - DNA, 20 bp, synthetic construct
  source 1..20: mol_type = other DNA
cttaggagggcagttggtcc

SEQ ID NO: 894 - DNA, 20 bp, synthetic construct
  source 1..20: mol_type = other DNA
aggaaagcaagtgtgggtcc

SEQ ID NO: 895 - DNA, 20 bp, synthetic construct
  source 1..20: mol_type = other DNA
aacacgaggaaagcaagtgt

SEQ ID NO: 896 - DNA, 20 bp, synthetic construct
  source 1..20: mol_type = other DNA
aaacacgaggaaagcaagtg

SEQ ID NO: 897 - DNA, 20 bp, synthetic construct
  source 1..20: mol_type = other DNA
aggcaggatcaggaaacacg

SEQ ID NO: 898 - DNA, 20 bp, synthetic construct
  source 1..20: mol_type = other DNA
tttcttcccacaggtggaaa

SEQ ID NO: 899 - DNA, 20 bp, synthetic construct
  source 1..20: mol_type = other DNA
cttcccacaggtggaaaagg

SEQ ID NO: 900 - DNA, 20 bp, synthetic construct
  source 1..20: mol_type = other DNA
ttcccacaggtggaaaagga

SEQ ID NO: 901 - DNA, 20 bp, synthetic construct
  source 1..20: mol_type = other DNA
ctccctccttttccacctgt

SEQ ID NO: 902 - DNA, 20 bp, synthetic construct
  source 1..20: mol_type = other DNA
gctccctccttttccacctg

SEQ ID NO: 903 - DNA, 20 bp, synthetic construct
  source 1..20: mol_type = other DNA
aaaaggagggagctactctc

SEQ ID NO: 904 - DNA, 20 bp, synthetic construct
  source 1..20: mol_type = other DNA
actctcaggctgcgtgtaag

SEQ ID NO: 905 - DNA, 20 bp, synthetic construct
  source 1..20: mol_type = other DNA
ctcaggctgcgtgtaagtgg

SEQ ID NO: 906 - DNA, 20 bp, synthetic construct
  source 1..20: mol_type = other DNA
tcaggctgcgtgtaagtggt

SEQ ID NO: 907 - DNA, 20 bp, synthetic construct
  source 1..20: mol_type = other DNA
caggctgcgtgtaagtggtg

SEQ ID NO: 908 - DNA, 20 bp, synthetic construct
  source 1..20: mol_type = other DNA
aggctgcgtgtaagtggtgg

SEQ ID NO: 909 - DNA, 20 bp, synthetic construct
  source 1..20: mol_type = other DNA
ctgcgtgtaagtggtggggg

SEQ ID NO: 910 - DNA, 20 bp, synthetic construct
  source 1..20: mol_type = other DNA
tgcgtgtaagtggtgggggt

SEQ ID NO: 911 - DNA, 20 bp, synthetic construct
  source 1..20: mol_type = other DNA
aagtggtgggggtgggagtg

SEQ ID NO: 912 - DNA, 20 bp, synthetic construct
  source 1..20: mol_type = other DNA
tggtgggggtgggagtgtgg

SEQ ID NO: 913 - DNA, 20 bp, synthetic construct
  source 1..20: mol_type = other DNA
gacaggaggaattatggggt

SEQ ID NO: 914 - DNA, 20 bp, synthetic construct
  source 1..20: mol_type = other DNA
ggacaggaggaattatgggg

SEQ ID NO: 915 - DNA, 20 bp, synthetic construct
  source 1..20: mol_type = other DNA
gtgggacaggaggaattatg

SEQ ID NO: 916 - DNA, 20 bp, synthetic construct
  source 1..20: mol_type = other DNA
cgtgggacaggaggaattat

SEQ ID NO: 917 - DNA, 20 bp, synthetic construct
  source 1..20: mol_type = other DNA
acgtgggacaggaggaatta

SEQ ID NO: 918 - DNA, 20 bp, synthetic construct
  source 1..20: mol_type = other DNA
cgcaggagacgtgggacagg

SEQ ID NO: 919 - DNA, 20 bp, synthetic construct
  source 1..20: mol_type = other DNA
ctcctgtcccacgtctcctg

SEQ ID NO: 920 - DNA, 20 bp, synthetic construct
  source 1..20: mol_type = other DNA
tcctgtcccacgtctcctgc

SEQ ID NO: 921 - DNA, 20 bp, synthetic construct
  source 1..20: mol_type = other DNA
gcccgcaggagacgtgggac

SEQ ID NO: 922 - DNA, 20 bp, synthetic construct
  source 1..20: mol_type = other DNA
tcagagcccgcaggagacgt

SEQ ID NO: 923 - DNA, 20 bp, synthetic construct
  source 1..20: mol_type = other DNA
ctagagtgggcggggggggc

SEQ ID NO: 924 - DNA, 20 bp, synthetic construct
  source 1..20: mol_type = other DNA
tagagtgggcggggggggcg

SEQ ID NO: 925 - DNA, 20 bp, synthetic construct
  source 1..20: mol_type = other DNA
agtgggcggggggggcgggg

SEQ ID NO: 926 - DNA, 20 bp, synthetic construct
  source 1..20: mol_type = other DNA
gtgggcggggggggcgggga

SEQ ID NO: 927 - DNA, 20 bp, synthetic construct
  source 1..20: mol_type = other DNA
tgggcggggggggcggggag

SEQ ID NO: 928 - DNA, 20 bp, synthetic construct
  source 1..20: mol_type = other DNA
gggcggggggggcggggagg

SEQ ID NO: 929 - DNA, 20 bp, synthetic construct
  source 1..20: mol_type = other DNA
ggggggcggggagggggcag

SEQ ID NO: 930 - DNA, 20 bp, synthetic construct
  source 1..20: mol_type = other DNA
gggggcggggagggggcaga

SEQ ID NO: 931 - DNA, 20 bp, synthetic construct
  source 1..20: mol_type = other DNA
ggggcggggagggggcagag

SEQ ID NO: 932 - DNA, 20 bp, synthetic construct
  source 1..20: mol_type = other DNA
ggggagggggcagaggggaa

SEQ ID NO: 933 - DNA, 20 bp, synthetic construct
  source 1..20: mol_type = other DNA
gggggcagaggggaaaggcc

SEQ ID NO: 934 - DNA, 20 bp, synthetic construct
  source 1..20: mol_type = other DNA
ggggcagaggggaaaggcct

SEQ ID NO: 935 - DNA, 20 bp, synthetic construct
  source 1..20: mol_type = other DNA
gaggggaaaggcctgggtaa

SEQ ID NO: 936 - DNA, 20 bp, synthetic construct
  source 1..20: mol_type = other DNA
tcaaagaatctccattaccc

SEQ ID NO: 937 - DNA, 20 bp, synthetic construct
  source 1..20: mol_type = other DNA
ggtaatggagattctttgat

SEQ ID NO: 938 - DNA, 20 bp, synthetic construct
  source 1..20: mol_type = other DNA
ggtaatggagattctttgat

SEQ ID NO: 939 - DNA, 20 bp, synthetic construct
  source 1..20: mol_type = other DNA
gtaatggagattctttgatt

SEQ ID NO: 940 - DNA, 20 bp, synthetic construct
  source 1..20: mol_type = other DNA
gggatgtttcgcgtgtgtcg

SEQ ID NO: 941 - DNA, 20 bp, synthetic construct
  source 1..20: mol_type = other DNA
ggatgtttcgcgtgtgtcgt

SEQ ID NO: 942 - DNA, 20 bp, synthetic construct
  source 1..20: mol_type = other DNA
gaacaaattctggttagtca

SEQ ID NO: 943 - DNA, 20 bp, synthetic construct
  source 1..20: mol_type = other DNA
caacagtcatgaacaaattc

SEQ ID NO: 944 - DNA, 20 bp, synthetic construct
  source 1..20: mol_type = other DNA
cctgagacagctgtcttgtg

SEQ ID NO: 945 - DNA, 20 bp, synthetic construct
  source 1..20: mol_type = other DNA
cctcacaagacagctgtctc

SEQ ID NO: 946 - DNA, 20 bp, synthetic construct
  source 1..20: mol_type = other DNA
ctgagacagctgtcttgtga

SEQ ID NO: 947 - DNA, 20 bp, synthetic construct
  source 1..20: mol_type = other DNA
cttgtgagggactgagatgc

SEQ ID NO: 948 - DNA, 20 bp, synthetic construct
  source 1..20: mol_type = other DNA
tcctcccctttgtgacttca

SEQ ID NO: 949 - DNA, 20 bp, synthetic construct
  source 1..20: mol_type = other DNA
cctcccctttgtgacttcaa

SEQ ID NO: 950 - DNA, 20 bp, synthetic construct
  source 1..20: mol_type = other DNA
cccttgaagtcacaaagggg

SEQ ID NO: 951 - DNA, 20 bp, synthetic construct
  source 1..20: mol_type = other DNA
aggcccttgaagtcacaaag

SEQ ID NO: 952 - DNA, 20 bp, synthetic construct
  source 1..20: mol_type = other DNA
gaggcccttgaagtcacaaa

SEQ ID NO: 953 - DNA, 20 bp, synthetic construct
  source 1..20: mol_type = other DNA
agaggcccttgaagtcacaa

SEQ ID NO: 954 - DNA, 20 bp, synthetic construct
  source 1..20: mol_type = other DNA
tttgtgacttcaagggcctc

SEQ ID NO: 955 - DNA, 20 bp, synthetic construct
  source 1..20: mol_type = other DNA
tttgcagaaagagatgccag

SEQ ID NO: 956 - DNA, 20 bp, synthetic construct
  source 1..20: mol_type = other DNA
tctggcatctctttctgcaa

SEQ ID NO: 957 - DNA, 20 bp, synthetic construct
  source 1..20: mol_type = other DNA
acagggacgcagacacattc

SEQ ID NO: 958 - DNA, 20 bp, synthetic construct
  source 1..20: mol_type = other DNA
gtccctgttagcctaatgtg

SEQ ID NO: 959 - DNA, 20 bp, synthetic construct
  source 1..20: mol_type = other DNA
ctcctcacattaggctaaca

SEQ ID NO: 960 - DNA, 20 bp, synthetic construct
  source 1..20: mol_type = other DNA
cctgttagcctaatgtgagg

SEQ ID NO: 961 - DNA, 20 bp, synthetic construct
  source 1..20: mol_type = other DNA
cctcctcacattaggctaac

SEQ ID NO: 962 - DNA, 20 bp, synthetic construct
  source 1..20: mol_type = other DNA
gttagcctaatgtgaggagg

SEQ ID NO: 963 - DNA, 20 bp, synthetic construct
  source 1..20: mol_type = other DNA
tctctccacctcctcacatt

SEQ ID NO: 964 - DNA, 20 bp, synthetic construct
  source 1..20: mol_type = other DNA
gtcacagtggacacgggggt

SEQ ID NO: 965 - DNA, 20 bp, synthetic construct
  source 1..20: mol_type = other DNA
ggtcacagtggacacggggg

SEQ ID NO: 966 - DNA, 20 bp, synthetic construct
  source 1..20: mol_type = other DNA
aggggtcacagtggacacgg

SEQ ID NO: 967 - DNA, 20 bp, synthetic construct
  source 1..20: mol_type = other DNA
gaaccgtcctcctgctgctc

SEQ ID NO: 968 - DNA, 20 bp, synthetic construct
  source 1..20: mol_type = other DNA
accgtcctcctgctgctctg

SEQ ID NO: 969 - DNA, 20 bp, synthetic construct
  source 1..20: mol_type = other DNA
ccgtcctcctgctgctctgg

SEQ ID NO: 970 - DNA, 20 bp, synthetic construct
  source 1..20: mol_type = other DNA
cgtcctcctgctgctctggg

SEQ ID NO: 971 - DNA, 20 bp, synthetic construct
  source 1..20: mol_type = other DNA
gccctgaccgagacctgggc

SEQ ID NO: 972 - DNA, 20 bp, synthetic construct
  source 1..20: mol_type = other DNA
accggcccaggtctcggtca

SEQ ID NO: 973 - DNA, 20 bp, synthetic construct
  source 1..20: mol_type = other DNA
caccggcccaggtctcggtc

SEQ ID NO: 974 - DNA, 20 bp, synthetic construct
  source 1..20: mol_type = other DNA
gcactcaccggcccaggtct

SEQ ID NO: 975 - DNA, 20 bp, synthetic construct
  source 1..20: mol_type = other DNA
gagacctgggccggtgagtg

SEQ ID NO: 976 - DNA, 20 bp, synthetic construct
  source 1..20: mol_type = other DNA
agacctgggccggtgagtgc

SEQ ID NO: 977 - DNA, 20 bp, synthetic construct
  source 1..20: mol_type = other DNA
gacctgggccggtgagtgcg

SEQ ID NO: 978 - DNA, 20 bp, synthetic construct
  source 1..20: mol_type = other DNA
gaccccgcactcaccggccc

SEQ ID NO: 979 - DNA, 20 bp, synthetic construct
  source 1..20: mol_type = other DNA
tgggccggtgagtgcggggt

SEQ ID NO: 980 - DNA, 20 bp, synthetic construct
  source 1..20: mol_type = other DNA
gggccggtgagtgcggggtc

SEQ ID NO: 981 - DNA, 20 bp, synthetic construct
  source 1..20: mol_type = other DNA
ccggtgagtgcggggtcggg

SEQ ID NO: 982 - DNA, 20 bp, synthetic construct
  source 1..20: mol_type = other DNA
cctcccgaccccgcactcac

SEQ ID NO: 983 - DNA, 20 bp, synthetic construct
  source 1..20: mol_type = other DNA
cggtgagtgcggggtcggga

SEQ ID NO: 984 - DNA, 20 bp, synthetic construct
  source 1..20: mol_type = other DNA
gtgcggggtcgggagggaaa

SEQ ID NO: 985 - DNA, 20 bp, synthetic construct
  source 1..20: mol_type = other DNA
cgggagggaaatggcctctg

SEQ ID NO: 986 - DNA, 20 bp, synthetic construct
  source 1..20: mol_type = other DNA
gggagggaaatggcctctgt

SEQ ID NO: 987 - DNA, 20 bp, synthetic construct
  source 1..20: mol_type = other DNA
ggagggaaatggcctctgtg

SEQ ID NO: 988 - DNA, 20 bp, synthetic construct
  source 1..20: mol_type = other DNA
gggaaatggcctctgtgggg

SEQ ID NO: 989 - DNA, 20 bp, synthetic construct
  source 1..20: mol_type = other DNA
ggcctctgtggggaggagcg

SEQ ID NO: 990 - DNA, 20 bp, synthetic construct
  source 1..20: mol_type = other DNA
gcctctgtggggaggagcga

SEQ ID NO: 991 - DNA, 20 bp, synthetic construct
  source 1..20: mol_type = other DNA
cctctgtggggaggagcgag

SEQ ID NO: 992 - DNA, 20 bp, synthetic construct
  source 1..20: mol_type = other DNA
cccctcgctcctccccacag

SEQ ID NO: 993 - DNA, 20 bp, synthetic construct
  source 1..20: mol_type = other DNA
gggaggagcgaggggaccgc

SEQ ID NO: 994 - DNA, 20 bp, synthetic construct
  source 1..20: mol_type = other DNA
aggagcgaggggaccgcagg

SEQ ID NO: 995 - DNA, 20 bp, synthetic construct
  source 1..20: mol_type = other DNA
ggagcgaggggaccgcaggc

SEQ ID NO: 996 - DNA, 20 bp, synthetic construct
  source 1..20: mol_type = other DNA
gagcgaggggaccgcaggcg

SEQ ID NO: 997 - DNA, 20 bp, synthetic construct
  source 1..20: mol_type = other DNA
agcgaggggaccgcaggcgg

SEQ ID NO: 998 - DNA, 20 bp, synthetic construct
  source 1..20: mol_type = other DNA
gggaccgcaggcgggggcgc

SEQ ID NO: 999 - DNA, 20 bp, synthetic construct
  source 1..20: mol_type = other DNA
aggtcctgcgcccccgcctg

SEQ ID NO: 1000 - DNA, 20 bp, synthetic construct
  source 1..20: mol_type = other DNA
aggcgggggcgcaggacctg

SEQ ID NO: 1001 - DNA, 20 bp, synthetic construct
  source 1..20: mol_type = other DNA
caggacctgaggagccgcgc

SEQ ID NO: 1002 - DNA, 20 bp, synthetic construct
  source 1..20: mol_type = other DNA
aggacctgaggagccgcgcc

SEQ ID NO: 1003 - DNA, 20 bp, synthetic construct
  source 1..20: mol_type = other DNA
acctgaggagccgcgccggg

SEQ ID NO: 1004 - DNA, 20 bp, synthetic construct
  source 1..20: mol_type = other DNA
tcctcccggcgcggctcctc

SEQ ID NO: 1005 - DNA, 20 bp, synthetic construct
  source 1..20: mol_type = other DNA
tgaggagccgcgccgggagg

SEQ ID NO: 1006 - DNA, 20 bp, synthetic construct
  source 1..20: mol_type = other DNA
gaggagccgcgccgggagga

SEQ ID NO: 1007 - DNA, 20 bp, synthetic construct
  source 1..20: mol_type = other DNA
agccgcgccgggaggagggt

SEQ ID NO: 1008 - DNA, 20 bp, synthetic construct
  source 1..20: mol_type = other DNA
tacctcatggagtgggagcc

SEQ ID NO: 1009 - DNA, 20 bp, synthetic construct
  source 1..20: mol_type = other DNA
ggacggagccccgggcgcca

SEQ ID NO: 1010 - DNA, 20 bp, synthetic construct
  source 1..20: mol_type = other DNA
agacttaccgagagaacctg

SEQ ID NO: 1011 - DNA, 20 bp, synthetic construct
  source 1..20: mol_type = other DNA
ggccctgaccgagacctggg

SEQ ID NO: 1012 - DNA, 20 bp, synthetic construct
  source 1..20: mol_type = other DNA
gtggaaatacctcatggagt

SEQ ID NO: 1013 - DNA, 20 bp, synthetic construct
  source 1..20: mol_type = other DNA
tgtggaaatacctcatggag

SEQ ID NO: 1014 - DNA, 20 bp, synthetic construct
  source 1..20: mol_type = other DNA
ggcggtgtggaaatacctca

SEQ ID NO: 1015 - DNA, 20 bp, synthetic construct
  source 1..20: mol_type = other DNA
atttccacaccgccatgtcc

SEQ ID NO: 1016 - DNA, 20 bp, synthetic construct
  source 1..20: mol_type = other DNA
cgggccgggacatggcggtg

SEQ ID NO: 1017 - DNA, 20 bp, synthetic construct
  source 1..20: mol_type = other DNA
cacaccgccatgtcccggcc

SEQ ID NO: 1018 - DNA, 20 bp, synthetic construct
  source 1..20: mol_type = other DNA
tggacgacacgctgttcgtg

SEQ ID NO: 1019 - DNA, 20 bp, synthetic construct
  source 1..20: mol_type = other DNA
cggctccttcctcggactcg

SEQ ID NO: 1020 - DNA, 20 bp, synthetic construct
  source 1..20: mol_type = other DNA
cgagtccgaggaaggagccg

SEQ ID NO: 1021 - DNA, 20 bp, synthetic construct
  source 1..20: mol_type = other DNA
gagtccgaggaaggagccgc

SEQ ID NO: 1022 - DNA, 20 bp, synthetic construct
  source 1..20: mol_type = other DNA
ggcgcccgcggctccttcct

SEQ ID NO: 1023 - DNA, 20 bp, synthetic construct
  source 1..20: mol_type = other DNA
ggaaggagccgcgggcgcca

SEQ ID NO: 1024 - DNA, 20 bp, synthetic construct
  source 1..20: mol_type = other DNA
gcgggcgccatggatagagc

SEQ ID NO: 1025 - DNA, 20 bp, synthetic construct
  source 1..20: mol_type = other DNA
gaggggccggagtattggga

SEQ ID NO: 1026 - DNA, 20 bp, synthetic construct
  source 1..20: mol_type = other DNA
cggggagacacggaacatga

SEQ ID NO: 1027 - DNA, 20 bp, synthetic construct
  source 1..20: mol_type = other DNA
ctctcggtaagtctgcgcgg

SEQ ID NO: 1028 - DNA, 20 bp, synthetic construct
  source 1..20: mol_type = other DNA
gttctctcggtaagtctgcg

SEQ ID NO: 1029 - DNA, 20 bp, synthetic construct
  source 1..20: mol_type = other DNA
tgtcttggaaaaaggctttg

SEQ ID NO: 1030 - DNA, 20 bp, synthetic construct
  source 1..20: mol_type = other DNA
aaaaaggctttgcggatccc

SEQ ID NO: 1031 - DNA, 20 bp, synthetic construct
  source 1..20: mol_type = other DNA
aaggctttgcggatccccgg

SEQ ID NO: 1032 - DNA, 20 bp, synthetic construct
  source 1..20: mol_type = other DNA
tgcggatccccggaggcctt

SEQ ID NO: 1033 - DNA, 20 bp, synthetic construct
  source 1..20: mol_type = other DNA
gcggatccccggaggccttc

SEQ ID NO: 1034 - DNA, 20 bp, synthetic construct
  source 1..20: mol_type = other DNA
ttgctgcccgaaggcctccg

SEQ ID NO: 1035 - DNA, 20 bp, synthetic construct
  source 1..20: mol_type = other DNA
gttgctgcccgaaggcctcc

SEQ ID NO: 1036 - DNA, 20 bp, synthetic construct
  source 1..20: mol_type = other DNA
agttgctgcccgaaggcctc

SEQ ID NO: 1037 - DNA, 20 bp, synthetic construct
  source 1..20: mol_type = other DNA
aggtcacagttgctgcccga

SEQ ID NO: 1038 - DNA, 20 bp, synthetic construct
  source 1..20: mol_type = other DNA
gctcagcatcgacagcatca

SEQ ID NO: 1039 - DNA, 20 bp, synthetic construct
  source 1..20: mol_type = other DNA
gtcgatgctgagcaccccag

SEQ ID NO: 1040 - DNA, 20 bp, synthetic construct
  source 1..20: mol_type = other DNA
gctgagcaccccagtggctg

SEQ ID NO: 1041 - DNA, 20 bp, synthetic construct
  source 1..20: mol_type = other DNA
ctgagcaccccagtggctga

SEQ ID NO: 1042 - DNA, 20 bp, synthetic construct
  source 1..20: mol_type = other DNA
gtctctgccctcagccactg

SEQ ID NO: 1043 - DNA, 20 bp, synthetic construct
  source 1..20: mol_type = other DNA
agtctctgccctcagccact

SEQ ID NO: 1044 - DNA, 20 bp, synthetic construct
  source 1..20: mol_type = other DNA
gagtctctgccctcagccac

SEQ ID NO: 1045 - DNA, 20 bp, synthetic construct
  source 1..20: mol_type = other DNA
ggatttcgtgtaccagttta

SEQ ID NO: 1046 - DNA, 20 bp, synthetic construct
  source 1..20: mol_type = other DNA
gatttcgtgtaccagtttaa

SEQ ID NO: 1047 - DNA, 20 bp, synthetic construct
  source 1..20: mol_type = other DNA
agtagcacatgcccttaaac

SEQ ID NO: 1048 - DNA, 20 bp, synthetic construct
  source 1..20: mol_type = other DNA
ggcatgtgctacttcaccaa

SEQ ID NO: 1049 - DNA, 20 bp, synthetic construct
  source 1..20: mol_type = other DNA
gcatgtgctacttcaccaac

SEQ ID NO: 1050 - DNA, 20 bp, synthetic construct
  source 1..20: mol_type = other DNA
acgcacgcgctctgtcccgt

SEQ ID NO: 1051 - DNA, 20 bp, synthetic construct
  source 1..20: mol_type = other DNA
cgaagcgcacgatctcttct

SEQ ID NO: 1052 - DNA, 20 bp, synthetic construct
  source 1..20: mol_type = other DNA
cgtgcgcttcgacagcgacg

SEQ ID NO: 1053 - DNA, 20 bp, synthetic construct
  source 1..20: mol_type = other DNA
gtgcgcttcgacagcgacgt

SEQ ID NO: 1054 - DNA, 20 bp, synthetic construct
  source 1..20: mol_type = other DNA
tgcgcttcgacagcgacgtg

SEQ ID NO: 1055 - DNA, 20 bp, synthetic construct
  source 1..20: mol_type = other DNA
gcgcttcgacagcgacgtgg

SEQ ID NO: 1056 - DNA, 20 bp, synthetic construct
  source 1..20: mol_type = other DNA
acagcgacgtgggggagttc

SEQ ID NO: 1057 - DNA, 20 bp, synthetic construct
  source 1..20: mol_type = other DNA
cagcgacgtgggggagttcc

SEQ ID NO: 1058 - DNA, 20 bp, synthetic construct
  source 1..20: mol_type = other DNA
cgacgtgggggagttccggg

SEQ ID NO: 1059 - DNA, 20 bp, synthetic construct
  source 1..20: mol_type = other DNA
gttccgggcggtgacgctgc

SEQ ID NO: 1060 - DNA, 20 bp, synthetic construct
  source 1..20: mol_type = other DNA
ttccgggcggtgacgctgct

SEQ ID NO: 1061 - DNA, 20 bp, synthetic construct
  source 1..20: mol_type = other DNA
tccgggcggtgacgctgctg

SEQ ID NO: 1062 - DNA, 20 bp, synthetic construct
  source 1..20: mol_type = other DNA
gccccagcagcgtcaccgcc

SEQ ID NO: 1063 - DNA, 20 bp, synthetic construct
  source 1..20: mol_type = other DNA
ggctgcctgccgccgagtac

SEQ ID NO: 1064 - DNA, 20 bp, synthetic construct
  source 1..20: mol_type = other DNA
ctgttccagtactcggcggc

SEQ ID NO: 1065 - DNA, 20 bp, synthetic construct
  source 1..20: mol_type = other DNA
ctggctgttccagtactcgg

SEQ ID NO: 1066 - DNA, 20 bp, synthetic construct
  source 1..20: mol_type = other DNA
cttctggctgttccagtact

SEQ ID NO: 1067 - DNA, 20 bp, synthetic construct
  source 1..20: mol_type = other DNA
cgagtactggaacagccaga

SEQ ID NO: 1068 - DNA, 20 bp, synthetic construct
  source 1..20: mol_type = other DNA
gaacagccagaaggacatcc

SEQ ID NO: 1069 - DNA, 20 bp, synthetic construct
  source 1..20: mol_type = other DNA
gccagaaggacatcctggag

SEQ ID NO: 1070 - DNA, 20 bp, synthetic construct
  source 1..20: mol_type = other DNA
tcctctccaggatgtccttc

SEQ ID NO: 1071 - DNA, 20 bp, synthetic construct
  source 1..20: mol_type = other DNA
aggacatcctggagaggaaa

SEQ ID NO: 1072 - DNA, 20 bp, synthetic construct
  source 1..20: mol_type = other DNA
ggacatcctggagaggaaac

SEQ ID NO: 1073 - DNA, 20 bp, synthetic construct
  source 1..20: mol_type = other DNA
catcctggagaggaaacggg

SEQ ID NO: 1074 - DNA, 20 bp, synthetic construct
  source 1..20: mol_type = other DNA
cctggagaggaaacgggcgg

SEQ ID NO: 1075 - DNA, 20 bp, synthetic construct
  source 1..20: mol_type = other DNA
ccgccgcccgtttcctctcc

SEQ ID NO: 1076 - DNA, 20 bp, synthetic construct
  source 1..20: mol_type = other DNA
ggagaggaaacgggcggcgg

SEQ ID NO: 1077 - DNA, 20 bp, synthetic construct
  source 1..20: mol_type = other DNA
ggaaacgggcggcggtggac

SEQ ID NO: 1078 - DNA, 20 bp, synthetic construct
  source 1..20: mol_type = other DNA
gaaacgggcggcggtggaca

SEQ ID NO: 1079 - DNA, 20 bp, synthetic construct
  source 1..20: mol_type = other DNA
gtgcagacacaactaccagt

SEQ ID NO: 1080 - DNA, 20 bp, synthetic construct
  source 1..20: mol_type = other DNA
aggtcgtgcggagctccaac

SEQ ID NO: 1081 - DNA, 20 bp, synthetic construct
  source 1..20: mol_type = other DNA
agctccgcacgaccttgcag

SEQ ID NO: 1082 - DNA, 20 bp, synthetic construct
  source 1..20: mol_type = other DNA
ctcgccgctgcaaggtcgtg

SEQ ID NO: 1083 - DNA, 20 bp, synthetic construct
  source 1..20: mol_type = other DNA
aggtaacagttgctgcccga

SEQ ID NO: 1084 - DNA, 20 bp, synthetic construct
  source 1..20: mol_type = other DNA
agcaactgttaccttgatgc

SEQ ID NO: 1085 - DNA, 20 bp, synthetic construct
  source 1..20: mol_type = other DNA
gctcagcatcgccagcatca

SEQ ID NO: 1086 - DNA, 20 bp, synthetic construct
  source 1..20: mol_type = other DNA
ggcgatgctgagcaccccag

SEQ ID NO: 1087 - DNA, 20 bp, synthetic construct
  source 1..20: mol_type = other DNA
agtagcacatggccttaaac

SEQ ID NO: 1088 - DNA, 20 bp, synthetic construct
  source 1..20: mol_type = other DNA
gccatgtgctacttcaccaa

SEQ ID NO: 1089 - DNA, 20 bp, synthetic construct
  source 1..20: mol_type = other DNA
ccatgtgctacttcaccaac

SEQ ID NO: 1090 - DNA, 20 bp, synthetic construct
  source 1..20: mol_type = other DNA
cccgttggtgaagtagcaca

SEQ ID NO: 1091 - DNA, 20 bp, synthetic construct
  source 1..20: mol_type = other DNA
gtgctacttcaccaacggga

SEQ ID NO: 1092 - DNA, 20 bp, synthetic construct
  source 1..20: mol_type = other DNA
acgcacgcgctccgtcccgt

SEQ ID NO: 1093 - DNA, 20 bp, synthetic construct
  source 1..20: mol_type = other DNA
ctctcggttatagatgtatc

SEQ ID NO: 1094 - DNA, 20 bp, synthetic construct
  source 1..20: mol_type = other DNA
cagatacatctataaccgag

SEQ ID NO: 1095 - DNA, 20 bp, synthetic construct
  source 1..20: mol_type = other DNA
cgaagcgtgcgtactcctct

SEQ ID NO: 1096 - DNA, 20 bp, synthetic construct
  source 1..20: mol_type = other DNA
cgcacgcttcgacagcgacg

SEQ ID NO: 1097 - DNA, 20 bp, synthetic construct
  source 1..20: mol_type = other DNA
acgcttcgacagcgacgtgg

SEQ ID NO: 1098 - DNA, 20 bp, synthetic construct
  source 1..20: mol_type = other DNA
acagcgacgtggaggtgtac

SEQ ID NO: 1099 - DNA, 20 bp, synthetic construct
  source 1..20: mol_type = other DNA
cagcgacgtggaggtgtacc

SEQ ID NO: 1100 - DNA, 20 bp, synthetic construct
  source 1..20: mol_type = other DNA
cgacgtggaggtgtaccggg

SEQ ID NO: 1101 - DNA, 20 bp, synthetic construct
  source 1..20: mol_type = other DNA
gtaccgggcggtgacgccgc

SEQ ID NO: 1102 - DNA, 20 bp, synthetic construct
  source 1..20: mol_type = other DNA
taccgggcggtgacgccgct

SEQ ID NO: 1103 - DNA, 20 bp, synthetic construct
  source 1..20: mol_type = other DNA
accgggcggtgacgccgctg

SEQ ID NO: 1104 - DNA, 20 bp, synthetic construct
  source 1..20: mol_type = other DNA
gccccagcggcgtcaccgcc

SEQ ID NO: 1105 - DNA, 20 bp, synthetic construct
  source 1..20: mol_type = other DNA
tcggcgtcaggcggccccag

SEQ ID NO: 1106 - DNA, 20 bp, synthetic construct
  source 1..20: mol_type = other DNA
ggccgcctgacgccgagtac

SEQ ID NO: 1107 - DNA, 20 bp, synthetic construct
  source 1..20: mol_type = other DNA
ttccagtactcggcgtcagg

SEQ ID NO: 1108 - DNA, 20 bp, synthetic construct
  source 1..20: mol_type = other DNA
ctgttccagtactcggcgtc

SEQ ID NO: 1109 - DNA, 20 bp, synthetic construct
  source 1..20: mol_type = other DNA
gaacagccagaaggaagtcc

SEQ ID NO: 1110 - DNA, 20 bp, synthetic construct
  source 1..20: mol_type = other DNA
gccagaaggaagtcctggag

SEQ ID NO: 1111 - DNA, 20 bp, synthetic construct
  source 1..20: mol_type = other DNA
tcctctccaggacttccttc

SEQ ID NO: 1112 - DNA, 20 bp, synthetic construct
  source 1..20: mol_type = other DNA
aggaagtcctggagaggacc

SEQ ID NO: 1113 - DNA, 20 bp, synthetic construct
  source 1..20: mol_type = other DNA
ggaagtcctggagaggaccc

SEQ ID NO: 1114 - DNA, 20 bp, synthetic construct
  source 1..20: mol_type = other DNA
agtcctggagaggacccggg

SEQ ID NO: 1115 - DNA, 20 bp, synthetic construct
  source 1..20: mol_type = other DNA
actccgcccgggtcctctcc

SEQ ID NO: 1116 - DNA, 20 bp, synthetic construct
  source 1..20: mol_type = other DNA
ggagaggacccgggcggagt

SEQ ID NO: 1117 - DNA, 20 bp, synthetic construct
  source 1..20: mol_type = other DNA
gacccgggcggagttggaca

SEQ ID NO: 1118 - DNA, 20 bp, synthetic construct
  source 1..20: mol_type = other DNA
caccgtgtccaactccgccc

SEQ ID NO: 1119 - DNA, 20 bp, synthetic construct
  source 1..20: mol_type = other DNA
acaccgtgtccaactccgcc

SEQ ID NO: 1120 - DNA, 20 bp, synthetic construct
  source 1..20: mol_type = other DNA
tgtcttggaagaaggctttg

SEQ ID NO: 1121 - DNA, 20 bp, synthetic construct
  source 1..20: mol_type = other DNA
aagaaggctttgcggatccc

SEQ ID NO: 1122 - DNA, 20 bp, synthetic construct
  source 1..20: mol_type = other DNA
aaggctttgcggatccctgg

SEQ ID NO: 1123 - DNA, 20 bp, synthetic construct
  source 1..20: mol_type = other DNA
tgcggatccctggaggcctt

SEQ ID NO: 1124 - DNA, 20 bp, synthetic construct
  source 1..20: mol_type = other DNA
gcggatccctggaggccttc

SEQ ID NO: 1125 - DNA, 20 bp, synthetic construct
  source 1..20: mol_type = other DNA
ttgctacccgaaggcctcca

SEQ ID NO: 1126 - DNA, 20 bp, synthetic construct
  source 1..20: mol_type = other DNA
gttgctacccgaaggcctcc

SEQ ID NO: 1127 - DNA, 20 bp, synthetic construct
  source 1..20: mol_type = other DNA
aggtcacagttgctacccga

SEQ ID NO: 1128 - DNA, 20 bp, synthetic construct
  source 1..20: mol_type = other DNA
agcaactgtgaccttgatgc

SEQ ID NO: 1129 - DNA, 20 bp, synthetic construct
  source 1..20: mol_type = other DNA
gctggcgatgctgagcaccc

SEQ ID NO: 1130 - DNA, 20 bp, synthetic construct
  source 1..20: mol_type = other DNA
ggcgatgctgagcaccccgg

SEQ ID NO: 1131 - DNA, 20 bp, synthetic construct
  source 1..20: mol_type = other DNA
gctgagcaccccggtggctg

SEQ ID NO: 1132 - DNA, 20 bp, synthetic construct
  source 1..20: mol_type = other DNA
ctgagcaccccggtggctga

SEQ ID NO: 1133 - DNA, 20 bp, synthetic construct
  source 1..20: mol_type = other DNA
gtctctgccctcagccaccg

SEQ ID NO: 1134 - DNA, 20 bp, synthetic construct
  source 1..20: mol_type = other DNA
agtctctgccctcagccacc

SEQ ID NO: 1135 - DNA, 20 bp, synthetic construct
  source 1..20: mol_type = other DNA
gcacgcttcgacagcgacgt

SEQ ID NO: 1136 - DNA, 20 bp, synthetic construct
  source 1..20: mol_type = other DNA
cacgcttcgacagcgacgtg

SEQ ID NO: 1137 - DNA, 20 bp, synthetic construct
  source 1..20: mol_type = other DNA
acagcgacgtgggggtgtat

SEQ ID NO: 1138 - DNA, 20 bp, synthetic construct
  source 1..20: mol_type = other DNA
cagcgacgtgggggtgtatc

SEQ ID NO: 1139 - DNA, 20 bp, synthetic construct
  source 1..20: mol_type = other DNA
cgacgtgggggtgtatcggg

SEQ ID NO: 1140 - DNA, 20 bp, synthetic construct
  source 1..20: mol_type = other DNA
gtatcgggcggtgacgccgc

SEQ ID NO: 1141 - DNA, 20 bp, synthetic construct
  source 1..20: mol_type = other DNA
tatcgggcggtgacgccgct

SEQ ID NO: 1142 - DNA, 20 bp, synthetic construct
  source 1..20: mol_type = other DNA
atcgggcggtgacgccgctg

SEQ ID NO: 1143 - DNA, 20 bp, synthetic construct
  source 1..20: mol_type = other DNA
tcggcggcaggcggccccag

SEQ ID NO: 1144 - DNA, 20 bp, synthetic construct
  source 1..20: mol_type = other DNA
ggccgcctgccgccgagtac

SEQ ID NO: 1145 - DNA, 20 bp, synthetic construct
  source 1..20: mol_type = other DNA
ttccagtactcggcggcagg

SEQ ID NO: 1146 - DNA, 20 bp, synthetic construct
  source 1..20: mol_type = other DNA
tgtcttggaagaagtctttg

SEQ ID NO: 1147 - DNA, 20 bp, synthetic construct
  source 1..20: mol_type = other DNA
aagaagtctttgcggatccc

SEQ ID NO: 1148 - DNA, 20 bp, synthetic construct
  source 1..20: mol_type = other DNA
tgcggatccccggagacctt

SEQ ID NO: 1149 - DNA, 20 bp, synthetic construct
  source 1..20: mol_type = other DNA
gcggatccccggagaccttc

SEQ ID NO: 1150 - DNA, 20 bp, synthetic construct
  source 1..20: mol_type = other DNA
ttgctacccgaaggtctccg

SEQ ID NO: 1151 - DNA, 20 bp, synthetic construct
  source 1..20: mol_type = other DNA
gttgctacccgaaggtctcc

SEQ ID NO: 1152 - DNA, 20 bp, synthetic construct
  source 1..20: mol_type = other DNA
agttgctacccgaaggtctc

SEQ ID NO: 1153 - DNA, 20 bp, synthetic construct
  source 1..20: mol_type = other DNA
aggtgacagttgctacccga

SEQ ID NO: 1154 - DNA, 20 bp, synthetic construct
  source 1..20: mol_type = other DNA
agcaactgtcaccttgatgc

SEQ ID NO: 1155 - DNA, 20 bp, synthetic construct
  source 1..20: mol_type = other DNA
gctcaggatcgccagcatca

SEQ ID NO: 1156 - DNA, 20 bp, synthetic construct
  source 1..20: mol_type = other DNA
ggcgatcctgagctcctcac

SEQ ID NO: 1157 - DNA, 20 bp, synthetic construct
  source 1..20: mol_type = other DNA
cctgagctcctcactggctg

SEQ ID NO: 1158 - DNA, 20 bp, synthetic construct
  source 1..20: mol_type = other DNA
cctcagccagtgaggagctc

SEQ ID NO: 1159 - DNA, 20 bp, synthetic construct
  source 1..20: mol_type = other DNA
ctgagctcctcactggctga

SEQ ID NO: 1160 - DNA, 20 bp, synthetic construct
  source 1..20: mol_type = other DNA
gtctctgccctcagccagtg

SEQ ID NO: 1161 - DNA, 20 bp, synthetic construct
  source 1..20: mol_type = other DNA
agtagcacaggcccttaaac

SEQ ID NO: 1162 - DNA, 20 bp, synthetic construct
  source 1..20: mol_type = other DNA
ggcctgtgctacttcaccaa

SEQ ID NO: 1163 - DNA, 20 bp, synthetic construct
  source 1..20: mol_type = other DNA
gcctgtgctacttcaccaac

SEQ ID NO: 1164 - DNA, 20 bp, synthetic construct
  source 1..20: mol_type = other DNA
tcccgttggtgaagtagcac

SEQ ID NO: 1165 - DNA, 20 bp, synthetic construct
  source 1..20: mol_type = other DNA
ccaacgggacggagcgcgtg

SEQ ID NO: 1166 - DNA, 20 bp, synthetic construct
  source 1..20: mol_type = other DNA
ccgcacgcgctccgtcccgt

SEQ ID NO: 1167 - DNA, 20 bp, synthetic construct
  source 1..20: mol_type = other DNA
caacgggacggagcgcgtgc

SEQ ID NO: 1168 - DNA, 20 bp, synthetic construct
  source 1..20: mol_type = other DNA
aacgggacggagcgcgtgcg

SEQ ID NO: 1169 - DNA, 20 bp, synthetic construct
  source 1..20: mol_type = other DNA
ctctcggttatagatgtgtc

SEQ ID NO: 1170 - DNA, 20 bp, synthetic construct
  source 1..20: mol_type = other DNA
cagacacatctataaccgag

SEQ ID NO: 1171 - DNA, 20 bp, synthetic construct
  source 1..20: mol_type = other DNA
cgaagcgcacgtactcctct

SEQ ID NO: 1172 - DNA, 20 bp, synthetic construct
  source 1..20: mol_type = other DNA
acagcgacgtgggggtgtac

SEQ ID NO: 1173 - DNA, 20 bp, synthetic construct
  source 1..20: mol_type = other DNA
cagcgacgtgggggtgtacc

SEQ ID NO: 1174 - DNA, 20 bp, synthetic construct
  source 1..20: mol_type = other DNA
gtaccgggcagtgacgccgc

SEQ ID NO: 1175 - DNA, 20 bp, synthetic construct
  source 1..20: mol_type = other DNA
taccgggcagtgacgccgca

SEQ ID NO: 1176 - DNA, 20 bp, synthetic construct
  source 1..20: mol_type = other DNA
accgggcagtgacgccgcag

SEQ ID NO: 1177 - DNA, 20 bp, synthetic construct
  source 1..20: mol_type = other DNA
gcccctgcggcgtcactgcc

SEQ ID NO: 1178 - DNA, 20 bp, synthetic construct
  source 1..20: mol_type = other DNA
gggcagtgacgccgcagggg

SEQ ID NO: 1179 - DNA, 20 bp, synthetic construct
  source 1..20: mol_type = other DNA
tcggcaacaggccgcccctg

SEQ ID NO: 1180 - DNA, 20 bp, synthetic construct
  source 1..20: mol_type = other DNA
ggcggcctgttgccgagtac

SEQ ID NO: 1181 - DNA, 20 bp, synthetic construct
  source 1..20: mol_type = other DNA
ctgttccagtactcggcaac

SEQ ID NO: 1182 - DNA, 20 bp, synthetic construct
  source 1..20: mol_type = other DNA
cagccagaaggaagtcctgg

SEQ ID NO: 1183 - DNA, 20 bp, synthetic construct
  source 1..20: mol_type = other DNA
agccagaaggaagtcctgga

SEQ ID NO: 1184 - DNA, 20 bp, synthetic construct
  source 1..20: mol_type = other DNA
ccagaaggaagtcctggagg

SEQ ID NO: 1185 - DNA, 20 bp, synthetic construct
  source 1..20: mol_type = other DNA
ccccctccaggacttccttc

SEQ ID NO: 1186 - DNA, 20 bp, synthetic construct
  source 1..20: mol_type = other DNA
aggaagtcctggagggggcc

SEQ ID NO: 1187 - DNA, 20 bp, synthetic construct
  source 1..20: mol_type = other DNA
ggaagtcctggagggggccc

SEQ ID NO: 1188 - DNA, 20 bp, synthetic construct
  source 1..20: mol_type = other DNA
cctggagggggcccgggcgt

SEQ ID NO: 1189 - DNA, 20 bp, synthetic construct
  source 1..20: mol_type = other DNA
ccgacgcccgggccccctcc

SEQ ID NO: 1190 - DNA, 20 bp, synthetic construct
  source 1..20: mol_type = other DNA
ggagggggcccgggcgtcgg

SEQ ID NO: 1191 - DNA, 20 bp, synthetic construct
  source 1..20: mol_type = other DNA
gggcccgggcgtcggtggac

SEQ ID NO: 1192 - DNA, 20 bp, synthetic construct
  source 1..20: mol_type = other DNA
ggcccgggcgtcggtggaca

SEQ ID NO: 1193 - DNA, 20 bp, synthetic construct
  source 1..20: mol_type = other DNA
caccctgtccaccgacgccc

SEQ ID NO: 1194 - DNA, 20 bp, synthetic construct
  source 1..20: mol_type = other DNA
acaccctgtccaccgacgcc

SEQ ID NO: 1195 - DNA, 20 bp, synthetic construct
  source 1..20: mol_type = other DNA
ggtgtgcagacacaactacg

SEQ ID NO: 1196 - DNA, 20 bp, synthetic construct
  source 1..20: mol_type = other DNA
gtgcagacacaactacgagg

SEQ ID NO: 1197 - DNA, 20 bp, synthetic construct
  source 1..20: mol_type = other DNA
aactacgaggtggcgtaccg

SEQ ID NO: 1198 - DNA, 20 bp, synthetic construct
  source 1..20: mol_type = other DNA
actacgaggtggcgtaccgc

SEQ ID NO: 1199 - DNA, 20 bp, synthetic construct
  source 1..20: mol_type = other DNA
cgtaccgcgggatcctgcag

SEQ ID NO: 1200 - DNA, 20 bp, synthetic construct
  source 1..20: mol_type = other DNA
ctctcctctgcaggatcccg

SEQ ID NO: 1201 - DNA, 20 bp, synthetic construct
  source 1..20: mol_type = other DNA
tatcgggcggtgacgccgca

SEQ ID NO: 1202 - DNA, 20 bp, synthetic construct
  source 1..20: mol_type = other DNA
atcgggcggtgacgccgcag

SEQ ID NO: 1203 - DNA, 20 bp, synthetic construct
  source 1..20: mol_type = other DNA
gggcggtgacgccgcagggg

SEQ ID NO: 1204 - DNA, 20 bp, synthetic construct
  source 1..20: mol_type = other DNA
tcggcgtcaggccgcccctg

SEQ ID NO: 1205 - DNA, 20 bp, synthetic construct
  source 1..20: mol_type = other DNA
ggcggcctgacgccgagtac

SEQ ID NO: 1206 - DNA, 20 bp, synthetic construct
  source 1..20: mol_type = other DNA
cactctgtccaccgacgccc

SEQ ID NO: 1207 - DNA, 20 bp, synthetic construct
  source 1..20: mol_type = other DNA
acactctgtccaccgacgcc

SEQ ID NO: 1208 - DNA, 20 bp, synthetic construct
  source 1..20: mol_type = other DNA
agtgtgcagacacaactacg

SEQ ID NO: 1209 - DNA, 20 bp, synthetic construct
  source 1..20: mol_type = other DNA
ggcgatgctgagctccctac

SEQ ID NO: 1210 - DNA, 20 bp, synthetic construct
  source 1..20: mol_type = other DNA
gctgagctccctactggctg

SEQ ID NO: 1211 - DNA, 20 bp, synthetic construct
  source 1..20: mol_type = other DNA
ctgagctccctactggctga

SEQ ID NO: 1212 - DNA, 20 bp, synthetic construct
  source 1..20: mol_type = other DNA
gtctctgccctcagccagta

SEQ ID NO: 1213 - DNA, 20 bp, synthetic construct
  source 1..20: mol_type = other DNA
agtctctgccctcagccagt

SEQ ID NO: 1214 - DNA, 20 bp, synthetic construct
  source 1..20: mol_type = other DNA
ggatttcgtgttccagttta

SEQ ID NO: 1215 - DNA, 20 bp, synthetic construct
  source 1..20: mol_type = other DNA
gatttcgtgttccagtttaa

SEQ ID NO: 1216 - DNA, 20 bp, synthetic construct
  source 1..20: mol_type = other DNA
cgaagcgcgcgtactcctct

SEQ ID NO: 1217 - DNA, 20 bp, synthetic construct
  source 1..20: mol_type = other DNA
cgcgcgcttcgacagcgacg

SEQ ID NO: 1218 - DNA, 20 bp, synthetic construct
  source 1..20: mol_type = other DNA
gcgcgcttcgacagcgacgt

SEQ ID NO: 1219 - DNA, 20 bp, synthetic construct
  source 1..20: mol_type = other DNA
cgcgcttcgacagcgacgtg

SEQ ID NO: 1220 - DNA, 20 bp, synthetic construct
  source 1..20: mol_type = other DNA
cgacgtgggggtgtaccgcg

SEQ ID NO: 1221 - DNA, 20 bp, synthetic construct
  source 1..20: mol_type = other DNA
gtaccgcgcggtgacgccgc

SEQ ID NO: 1222 - DNA, 20 bp, synthetic construct
  source 1..20: mol_type = other DNA
taccgcgcggtgacgccgca

SEQ ID NO: 1223 - DNA, 20 bp, synthetic construct
  source 1..20: mol_type = other DNA
accgcgcggtgacgccgcag

SEQ ID NO: 1224 - DNA, 20 bp, synthetic construct
  source 1..20: mol_type = other DNA
gcccctgcggcgtcaccgcg

SEQ ID NO: 1225 - DNA, 20 bp, synthetic construct
  source 1..20: mol_type = other DNA
gcgcggtgacgccgcagggg

SEQ ID NO: 1226 - DNA, 20 bp, synthetic construct
  source 1..20: mol_type = other DNA
tcggcatcaggccgcccctg

SEQ ID NO: 1227 - DNA, 20 bp, synthetic construct
  source 1..20: mol_type = other DNA
ggcggcctgatgccgagtac

SEQ ID NO: 1228 - DNA, 20 bp, synthetic construct
  source 1..20: mol_type = other DNA
ctgttccagtactcggcatc

SEQ ID NO: 1229 - DNA, 20 bp, synthetic construct
  source 1..20: mol_type = other DNA
tcccctccaggacttccttc

SEQ ID NO: 1230 - DNA, 20 bp, synthetic construct
  source 1..20: mol_type = other DNA
aggaagtcctggaggggacc

SEQ ID NO: 1231 - DNA, 20 bp, synthetic construct
  source 1..20: mol_type = other DNA
ggaagtcctggaggggaccc

SEQ ID NO: 1232 - DNA, 20 bp, synthetic construct
  source 1..20: mol_type = other DNA
agtcctggaggggacccggg

SEQ ID NO: 1233 - DNA, 20 bp, synthetic construct
  source 1..20: mol_type = other DNA
actccgcccgggtcccctcc

SEQ ID NO: 1234 - DNA, 20 bp, synthetic construct
  source 1..20: mol_type = other DNA
ggaggggacccgggcggagt

SEQ ID NO: 1235 - DNA, 20 bp, synthetic construct
  source 1..20: mol_type = other DNA
aactacgaggtggcgttccg

SEQ ID NO: 1236 - DNA, 20 bp, synthetic construct
  source 1..20: mol_type = other DNA
actacgaggtggcgttccgc

SEQ ID NO: 1237 - DNA, 20 bp, synthetic construct
  source 1..20: mol_type = other DNA
cgttccgcgggatcttgcag

SEQ ID NO: 1238 - DNA, 20 bp, synthetic construct
  source 1..20: mol_type = other DNA
ctctcctctgcaagatcccg

SEQ ID NO: 1239 - DNA, 20 bp, synthetic construct
  source 1..20: mol_type = other DNA
atggtgtgtctgaagctccc

SEQ ID NO: 1240 - DNA, 20 bp, synthetic construct
  source 1..20: mol_type = other DNA
gtgtgtctgaagctccctgg

SEQ ID NO: 1241 - DNA, 20 bp, synthetic construct
  source 1..20: mol_type = other DNA
ctgtcatgcaggagcctcca

SEQ ID NO: 1242 - DNA, 20 bp, synthetic construct
  source 1..20: mol_type = other DNA
gctgtcatgcaggagcctcc

SEQ ID NO: 1243 - DNA, 20 bp, synthetic construct
  source 1..20: mol_type = other DNA
cactgtcagcgctgtcatgc

SEQ ID NO: 1244 - DNA, 20 bp, synthetic construct
  source 1..20: mol_type = other DNA
agcgctgacagtgacactga

SEQ ID NO: 1245 - DNA, 20 bp, synthetic construct
  source 1..20: mol_type = other DNA
gatggtgctgagctccccac

SEQ ID NO: 1246 - DNA, 20 bp, synthetic construct
  source 1..20: mol_type = other DNA
gctgagctccccactggctt

SEQ ID NO: 1247 - DNA, 20 bp, synthetic construct
  source 1..20: mol_type = other DNA
agctccccactggctttggc

SEQ ID NO: 1248 - DNA, 20 bp, synthetic construct
  source 1..20: mol_type = other DNA
gctccccactggctttggct

SEQ ID NO: 1249 - DNA, 20 bp, synthetic construct
  source 1..20: mol_type = other DNA
ctccccactggctttggctg

SEQ ID NO: 1250 - DNA, 20 bp, synthetic construct
  source 1..20: mol_type = other DNA
gtccccagccaaagccagtg

SEQ ID NO: 1251 - DNA, 20 bp, synthetic construct
  source 1..20: mol_type = other DNA
tgtccccagccaaagccagt

SEQ ID NO: 1252 - DNA, 20 bp, synthetic construct
  source 1..20: mol_type = other DNA
gtgtccccagccaaagccag

SEQ ID NO: 1253 - DNA, 20 bp, synthetic construct
  source 1..20: mol_type = other DNA
tttgaatgtcatttcttcaa

SEQ ID NO: 1254 - DNA, 20 bp, synthetic construct
  source 1..20: mol_type = other DNA
ttgaatgtcatttcttcaat

SEQ ID NO: 1255 - DNA, 20 bp, synthetic construct
  source 1..20: mol_type = other DNA
atgtcatttcttcaatggga

SEQ ID NO: 1256 - DNA, 20 bp, synthetic construct
  source 1..20: mol_type = other DNA
atttcttcaatgggacggag

SEQ ID NO: 1257 - DNA, 20 bp, synthetic construct
  source 1..20: mol_type = other DNA
tttcttcaatgggacggagc

SEQ ID NO: 1258 - DNA, 20 bp, synthetic construct
  source 1..20: mol_type = other DNA
tcaatgggacggagcgggtg

SEQ ID NO: 1259 - DNA, 20 bp, synthetic construct
  source 1..20: mol_type = other DNA
gacggagcgggtgcggttgc

SEQ ID NO: 1260 - DNA, 20 bp, synthetic construct
  source 1..20: mol_type = other DNA
aagatgcatctataaccaag

SEQ ID NO: 1261 - DNA, 20 bp, synthetic construct
  source 1..20: mol_type = other DNA
cgaagcgcacggactcctct

SEQ ID NO: 1262 - DNA, 20 bp, synthetic construct
  source 1..20: mol_type = other DNA
cacgtcgctgtcgaagcgca

SEQ ID NO: 1263 - DNA, 20 bp, synthetic construct
  source 1..20: mol_type = other DNA
acagcgacgtgggggagtac

SEQ ID NO: 1264 - DNA, 20 bp, synthetic construct
  source 1..20: mol_type = other DNA
cagcgacgtgggggagtacc

SEQ ID NO: 1265 - DNA, 20 bp, synthetic construct
  source 1..20: mol_type = other DNA
cgacgtgggggagtaccggg

SEQ ID NO: 1266 - DNA, 20 bp, synthetic construct
  source 1..20: mol_type = other DNA
gggggagtaccgggcggtga

SEQ ID NO: 1267 - DNA, 20 bp, synthetic construct
  source 1..20: mol_type = other DNA
gtaccgggcggtgacggagc

SEQ ID NO: 1268 - DNA, 20 bp, synthetic construct
  source 1..20: mol_type = other DNA
taccgggcggtgacggagct

SEQ ID NO: 1269 - DNA, 20 bp, synthetic construct
  source 1..20: mol_type = other DNA
accgggcggtgacggagctg

SEQ ID NO: 1270 - DNA, 20 bp, synthetic construct
  source 1..20: mol_type = other DNA
gccccagctccgtcaccgcc

SEQ ID NO: 1271 - DNA, 20 bp, synthetic construct
  source 1..20: mol_type = other DNA
gggcggtgacggagctgggg

SEQ ID NO: 1272 - DNA, 20 bp, synthetic construct
  source 1..20: mol_type = other DNA
gaacagccagaaggacctcc

SEQ ID NO: 1273 - DNA, 20 bp, synthetic construct
  source 1..20: mol_type = other DNA
tctgctccaggaggtccttc

SEQ ID NO: 1274 - DNA, 20 bp, synthetic construct
  source 1..20: mol_type = other DNA
agaaggacctcctggagcag

SEQ ID NO: 1275 - DNA, 20 bp, synthetic construct
  source 1..20: mol_type = other DNA
aggacctcctggagcagagg

SEQ ID NO: 1276 - DNA, 20 bp, synthetic construct
  source 1..20: mol_type = other DNA
ggacctcctggagcagaggc

SEQ ID NO: 1277 - DNA, 20 bp, synthetic construct
  source 1..20: mol_type = other DNA
cggcccgcctctgctccagg

SEQ ID NO: 1278 - DNA, 20 bp, synthetic construct
  source 1..20: mol_type = other DNA
cctggagcagaggcgggccg

SEQ ID NO: 1279 - DNA, 20 bp, synthetic construct
  source 1..20: mol_type = other DNA
ccgcggcccgcctctgctcc

SEQ ID NO: 1280 - DNA, 20 bp, synthetic construct
  source 1..20: mol_type = other DNA
ggagcagaggcgggccgcgg

SEQ ID NO: 1281 - DNA, 20 bp, synthetic construct
  source 1..20: mol_type = other DNA
tctgcagtaggtgtccaccg

SEQ ID NO: 1282 - DNA, 20 bp, synthetic construct
  source 1..20: mol_type = other DNA
acctactgcagacacaacta

SEQ ID NO: 1283 - DNA, 20 bp, synthetic construct
  source 1..20: mol_type = other DNA
cctactgcagacacaactac

SEQ ID NO: 1284 - DNA, 20 bp, synthetic construct
  source 1..20: mol_type = other DNA
cccgtagttgtgtctgcagt

SEQ ID NO: 1285 - DNA, 20 bp, synthetic construct
  source 1..20: mol_type = other DNA
ctactgcagacacaactacg

SEQ ID NO: 1286 - DNA, 20 bp, synthetic construct
  source 1..20: mol_type = other DNA
tgcagacacaactacggggt

SEQ ID NO: 1287 - DNA, 20 bp, synthetic construct
  source 1..20: mol_type = other DNA
gtgagagcttcacagtgcag

SEQ ID NO: 1288 - DNA, 20 bp, synthetic construct
  source 1..20: mol_type = other DNA
tctgcaataggtgtccaccg

SEQ ID NO: 1289 - DNA, 20 bp, synthetic construct
  source 1..20: mol_type = other DNA
acctattgcagacacaacta

SEQ ID NO: 1290 - DNA, 20 bp, synthetic construct
  source 1..20: mol_type = other DNA
cctattgcagacacaactac

SEQ ID NO: 1291 - DNA, 20 bp, synthetic construct
  source 1..20: mol_type = other DNA
cccgtagttgtgtctgcaat

SEQ ID NO: 1292 - DNA, 20 bp, synthetic construct
  source 1..20: mol_type = other DNA
ctattgcagacacaactacg

SEQ ID NO: 1293 - DNA, 20 bp, synthetic construct
  source 1..20: mol_type = other DNA
cagacacaactacggggctg

SEQ ID NO: 1294 - DNA, 20 bp, synthetic construct
  source 1..20: mol_type = other DNA
tggagagcttcacagtgcag

SEQ ID NO: 1295 - DNA, 20 bp, synthetic construct
  source 1..20: mol_type = other DNA
atggtgtgtctgaggctccc

SEQ ID NO: 1296 - DNA, 20 bp, synthetic construct
  source 1..20: mol_type = other DNA
gtgtgtctgaggctccctgg

SEQ ID NO: 1297 - DNA, 20 bp, synthetic construct
  source 1..20: mol_type = other DNA
gctccctggaggctcctgca

SEQ ID NO: 1298 - DNA, 20 bp, synthetic construct
  source 1..20: mol_type = other DNA
ctgccatgcaggagcctcca

SEQ ID NO: 1299 - DNA, 20 bp, synthetic construct
  source 1..20: mol_type = other DNA
actgccatgcaggagcctcc

SEQ ID NO: 1300 - DNA, 20 bp, synthetic construct
  source 1..20: mol_type = other DNA
cactgtcagaactgccatgc

SEQ ID NO: 1301 - DNA, 20 bp, synthetic construct
  source 1..20: mol_type = other DNA
agttctgacagtgacactga

SEQ ID NO: 1302 - DNA, 20 bp, synthetic construct
  source 1..20: mol_type = other DNA
tctgagtgtcatttcttcaa

SEQ ID NO: 1303 - DNA, 20 bp, synthetic construct
  source 1..20: mol_type = other DNA
ctgagtgtcatttcttcaat

SEQ ID NO: 1304 - DNA, 20 bp, synthetic construct
  source 1..20: mol_type = other DNA
gtgtcatttcttcaatggga

SEQ ID NO: 1305 - DNA, 20 bp, synthetic construct
  source 1..20: mol_type = other DNA
gacggagcgggtgcggtacc

SEQ ID NO: 1306 - DNA, 20 bp, synthetic construct
  source 1..20: mol_type = other DNA
ggttatggaagtatctgtcc

SEQ ID NO: 1307 - DNA, 20 bp, synthetic construct
  source 1..20: mol_type = other DNA
ggacagatacttccataacc

SEQ ID NO: 1308 - DNA, 20 bp, synthetic construct
  source 1..20: mol_type = other DNA
cagatacttccataaccagg

SEQ ID NO: 1309 - DNA, 20 bp, synthetic construct
  source 1..20: mol_type = other DNA
gcacgttctcctcctggtta

SEQ ID NO: 1310 - DNA, 20 bp, synthetic construct
  source 1..20: mol_type = other DNA
cgaagcgcacgttctcctcc

SEQ ID NO: 1311 - DNA, 20 bp, synthetic construct
  source 1..20: mol_type = other DNA
gggggagttccgggcggtga

SEQ ID NO: 1312 - DNA, 20 bp, synthetic construct
  source 1..20: mol_type = other DNA
gttccgggcggtgacggagc

SEQ ID NO: 1313 - DNA, 20 bp, synthetic construct
  source 1..20: mol_type = other DNA
ttccgggcggtgacggagct

SEQ ID NO: 1314 - DNA, 20 bp, synthetic construct
  source 1..20: mol_type = other DNA
tccgggcggtgacggagctg

SEQ ID NO: 1315 - DNA, 20 bp, synthetic construct
  source 1..20: mol_type = other DNA
aggacctcctggagcagaag

SEQ ID NO: 1316 - DNA, 20 bp, synthetic construct
  source 1..20: mol_type = other DNA
ggacctcctggagcagaagc

SEQ ID NO: 1317 - DNA, 20 bp, synthetic construct
  source 1..20: mol_type = other DNA
gacctcctggagcagaagcg

SEQ ID NO: 1318 - DNA, 20 bp, synthetic construct
  source 1..20: mol_type = other DNA
ggccccgcttctgctccagg

SEQ ID NO: 1319 - DNA, 20 bp, synthetic construct
  source 1..20: mol_type = other DNA
tcctggagcagaagcggggc

SEQ ID NO: 1320 - DNA, 20 bp, synthetic construct
  source 1..20: mol_type = other DNA
cctggagcagaagcggggcc

SEQ ID NO: 1321 - DNA, 20 bp, synthetic construct
  source 1..20: mol_type = other DNA
cccggccccgcttctgctcc

SEQ ID NO: 1322 - DNA, 20 bp, synthetic construct
  source 1..20: mol_type = other DNA
ggagcagaagcggggccggg

SEQ ID NO: 1323 - DNA, 20 bp, synthetic construct
  source 1..20: mol_type = other DNA
gtctgcagtagttgtccacc

SEQ ID NO: 1324 - DNA, 20 bp, synthetic construct
  source 1..20: mol_type = other DNA
aactactgcagacacaacta

SEQ ID NO: 1325 - DNA, 20 bp, synthetic construct
  source 1..20: mol_type = other DNA
actactgcagacacaactac

SEQ ID NO: 1326 - DNA, 20 bp, synthetic construct
  source 1..20: mol_type = other DNA
cagacacaactacggggttg

SEQ ID NO: 1327 - DNA, 20 bp, synthetic construct
  source 1..20: mol_type = other DNA
atggtgtgtctgaagttccc

SEQ ID NO: 1328 - DNA, 20 bp, synthetic construct
  source 1..20: mol_type = other DNA
gtgtgtctgaagttccctgg

SEQ ID NO: 1329 - DNA, 20 bp, synthetic construct
  source 1..20: mol_type = other DNA
gttccctggaggctcctgca

SEQ ID NO: 1330 - DNA, 20 bp, synthetic construct
  source 1..20: mol_type = other DNA
gctgccatgcaggagcctcc

SEQ ID NO: 1331 - DNA, 20 bp, synthetic construct
  source 1..20: mol_type = other DNA
cactgtcagagctgccatgc

SEQ ID NO: 1332 - DNA, 20 bp, synthetic construct
  source 1..20: mol_type = other DNA
agctctgacagtgacactga

SEQ ID NO: 1333 - DNA, 20 bp, synthetic construct
  source 1..20: mol_type = other DNA
catgagtgtcatttcttcaa

SEQ ID NO: 1334 - DNA, 20 bp, synthetic construct
  source 1..20: mol_type = other DNA
atgagtgtcatttcttcaac

SEQ ID NO: 1335 - DNA, 20 bp, synthetic construct
  source 1..20: mol_type = other DNA
gtgtcatttcttcaacggga

SEQ ID NO: 1336 - DNA, 20 bp, synthetic construct
  source 1..20: mol_type = other DNA
atttcttcaacgggacggag

SEQ ID NO: 1337 - DNA, 20 bp, synthetic construct
  source 1..20: mol_type = other DNA
tttcttcaacgggacggagc

SEQ ID NO: 1338 - DNA, 20 bp, synthetic construct
  source 1..20: mol_type = other DNA
tcaacgggacggagcgggtg

SEQ ID NO: 1339 - DNA, 20 bp, synthetic construct
  source 1..20: mol_type = other DNA
gacggagcgggtgcggttcc

SEQ ID NO: 1340 - DNA, 20 bp, synthetic construct
  source 1..20: mol_type = other DNA
ggtgatagaagtatctgtcc

SEQ ID NO: 1341 - DNA, 20 bp, synthetic construct
  source 1..20: mol_type = other DNA
cagatacttctatcaccaag

SEQ ID NO: 1342 - DNA, 20 bp, synthetic construct
  source 1..20: mol_type = other DNA
cggcccgcttctgctccagg

SEQ ID NO: 1343 - DNA, 20 bp, synthetic construct
  source 1..20: mol_type = other DNA
cctggagcagaagcgggccg

SEQ ID NO: 1344 - DNA, 20 bp, synthetic construct
  source 1..20: mol_type = other DNA
ccgcggcccgcttctgctcc

SEQ ID NO: 1345 - DNA, 20 bp, synthetic construct
  source 1..20: mol_type = other DNA
ggagcagaagcgggccgcgg

SEQ ID NO: 1346 - DNA, 20 bp, synthetic construct
  source 1..20: mol_type = other DNA
cgtcttccaggatgtccttc

SEQ ID NO: 1347 - DNA, 20 bp, synthetic construct
  source 1..20: mol_type = other DNA
aggacatcctggaagacgag

SEQ ID NO: 1348 - DNA, 20 bp, synthetic construct
  source 1..20: mol_type = other DNA
ggacatcctggaagacgagc

SEQ ID NO: 1349 - DNA, 20 bp, synthetic construct
  source 1..20: mol_type = other DNA
cctggaagacgagcgggccg

SEQ ID NO: 1350 - DNA, 20 bp, synthetic construct
  source 1..20: mol_type = other DNA
ccgcggcccgctcgtcttcc

SEQ ID NO: 1351 - DNA, 20 bp, synthetic construct
  source 1..20: mol_type = other DNA
ggaagacgagcgggccgcgg

SEQ ID NO: 1352 - DNA, 20 bp, synthetic construct
  source 1..20: mol_type = other DNA
acttatacttaccctgccac

SEQ ID NO: 1353 - DNA, 20 bp, synthetic construct
  source 1..20: mol_type = other DNA
tataagtgtcatttcttcaa

SEQ ID NO: 1354 - DNA, 20 bp, synthetic construct
  source 1..20: mol_type = other DNA
ataagtgtcatttcttcaac

SEQ ID NO: 1355 - DNA, 20 bp, synthetic construct
  source 1..20: mol_type = other DNA
gacggagcgggtgcagttcc

SEQ ID NO: 1356 - DNA, 20 bp, synthetic construct
  source 1..20: mol_type = other DNA
ggttatagaagagtctttcc

SEQ ID NO: 1357 - DNA, 20 bp, synthetic construct
  source 1..20: mol_type = other DNA
ggaaagactcttctataacc

SEQ ID NO: 1358 - DNA, 20 bp, synthetic construct
  source 1..20: mol_type = other DNA
aagactcttctataaccagg

SEQ ID NO: 1359 - DNA, 20 bp, synthetic construct
  source 1..20: mol_type = other DNA
cgaagcgcacgaactcctcc

SEQ ID NO: 1360 - DNA, 20 bp, synthetic construct
  source 1..20: mol_type = other DNA
accgggcggtgacggagcta

SEQ ID NO: 1361 - DNA, 20 bp, synthetic construct
  source 1..20: mol_type = other DNA
gccctagctccgtcaccgcc

SEQ ID NO: 1362 - DNA, 20 bp, synthetic construct
  source 1..20: mol_type = other DNA
gggcggtgacggagctaggg

SEQ ID NO: 1363 - DNA, 20 bp, synthetic construct
  source 1..20: mol_type = other DNA
ggcggcctgtcgccgagtcc

SEQ ID NO: 1364 - DNA, 20 bp, synthetic construct
  source 1..20: mol_type = other DNA
ctgttccaggactcggcgac

SEQ ID NO: 1365 - DNA, 20 bp, synthetic construct
  source 1..20: mol_type = other DNA
cttctggctgttccaggact

SEQ ID NO: 1366 - DNA, 20 bp, synthetic construct
  source 1..20: mol_type = other DNA
cgagtcctggaacagccaga

SEQ ID NO: 1367 - DNA, 20 bp, synthetic construct
  source 1..20: mol_type = other DNA
gatgtccttctggctgttcc

SEQ ID NO: 1368 - DNA, 20 bp, synthetic construct
  source 1..20: mol_type = other DNA
cagccagaaggacatcctgg

SEQ ID NO: 1369 - DNA, 20 bp, synthetic construct
  source 1..20: mol_type = other DNA
tgtcctccaggatgtccttc

SEQ ID NO: 1370 - DNA, 20 bp, synthetic construct
  source 1..20: mol_type = other DNA
agaaggacatcctggaggac

SEQ ID NO: 1371 - DNA, 20 bp, synthetic construct
  source 1..20: mol_type = other DNA
aggacatcctggaggacagg

SEQ ID NO: 1372 - DNA, 20 bp, synthetic construct
  source 1..20: mol_type = other DNA
ggacatcctggaggacaggc

SEQ ID NO: 1373 - DNA, 20 bp, synthetic construct
  source 1..20: mol_type = other DNA
gacatcctggaggacaggcg

SEQ ID NO: 1374 - DNA, 20 bp, synthetic construct
  source 1..20: mol_type = other DNA
cctggaggacaggcggggcc

SEQ ID NO: 1375 - DNA, 20 bp, synthetic construct
  source 1..20: mol_type = other DNA
cctggccccgcctgtcctcc

SEQ ID NO: 1376 - DNA, 20 bp, synthetic construct
  source 1..20: mol_type = other DNA
cctggccccgcctgtcctcc

SEQ ID NO: 1377 - DNA, 20 bp, synthetic construct
  source 1..20: mol_type = other DNA
ggaggacaggcggggccagg

SEQ ID NO: 1378 - DNA, 20 bp, synthetic construct
  source 1..20: mol_type = other DNA
gtctgcacacggtgtccacc

SEQ ID NO: 1379 - DNA, 20 bp, synthetic construct
  source 1..20: mol_type = other DNA
accgtgtgcagacacaacta

SEQ ID NO: 1380 - DNA, 20 bp, synthetic construct
  source 1..20: mol_type = other DNA
ccgtgtgcagacacaactac

SEQ ID NO: 1381 - DNA, 20 bp, synthetic construct
  source 1..20: mol_type = other DNA
cccgtagttgtgtctgcaca

SEQ ID NO: 1382 - DNA, 20 bp, synthetic construct
  source 1..20: mol_type = other DNA
cgtgtgcagacacaactacg

SEQ ID NO: 1383 - DNA, 20 bp, synthetic construct
  source 1..20: mol_type = other DNA
ggttatagaagtatctgtcc

SEQ ID NO: 1384 - DNA, 20 bp, synthetic construct
  source 1..20: mol_type = other DNA
cagatacttctataaccaag

SEQ ID NO: 1385 - DNA, 20 bp, synthetic construct
  source 1..20: mol_type = other DNA
ggagctggggcggcctgatg

SEQ ID NO: 1386 - DNA, 20 bp, synthetic construct
  source 1..20: mol_type = other DNA
ggcggcctgatgaggagtac

SEQ ID NO: 1387 - DNA, 20 bp, synthetic construct
  source 1..20: mol_type = other DNA
ctgttccagtactcctcatc

SEQ ID NO: 1388 - DNA, 20 bp, synthetic construct
  source 1..20: mol_type = other DNA
ggagtactggaacagccaga

SEQ ID NO: 1389 - DNA, 20 bp, synthetic construct
  source 1..20: mol_type = other DNA
gaacagccagaaggacttcc

SEQ ID NO: 1390 - DNA, 20 bp, synthetic construct
  source 1..20: mol_type = other DNA
tgtcttccaggaagtccttc

SEQ ID NO: 1391 - DNA, 20 bp, synthetic construct
  source 1..20: mol_type = other DNA
agaaggacttcctggaagac

SEQ ID NO: 1392 - DNA, 20 bp, synthetic construct
  source 1..20: mol_type = other DNA
aggacttcctggaagacagg

SEQ ID NO: 1393 - DNA, 20 bp, synthetic construct
  source 1..20: mol_type = other DNA
ggacttcctggaagacaggc

SEQ ID NO: 1394 - DNA, 20 bp, synthetic construct
  source 1..20: mol_type = other DNA
cctggaagacaggcgggccg

SEQ ID NO: 1395 - DNA, 20 bp, synthetic construct
  source 1..20: mol_type = other DNA
ccgcggcccgcctgtcttcc

SEQ ID NO: 1396 - DNA, 20 bp, synthetic construct
  source 1..20: mol_type = other DNA
ggaagacaggcgggccgcgg

SEQ ID NO: 1397 - DNA, 20 bp, synthetic construct
  source 1..20: mol_type = other DNA
gcacgaactcctcctggtta

SEQ ID NO: 1398 - DNA, 20 bp, synthetic construct
  source 1..20: mol_type = other DNA
ggagctggggcggcctgctg

SEQ ID NO: 1399 - DNA, 20 bp, synthetic construct
  source 1..20: mol_type = other DNA
ggcggcctgctgcggagcac

SEQ ID NO: 1400 - DNA, 20 bp, synthetic construct
  source 1..20: mol_type = other DNA
ctgttccagtgctccgcagc

SEQ ID NO: 1401 - DNA, 20 bp, synthetic construct
  source 1..20: mol_type = other DNA
ggagcactggaacagccaga

SEQ ID NO: 1402 - DNA, 20 bp, synthetic construct
  source 1..20: mol_type = other DNA
gccagaaggacctcctggag

SEQ ID NO: 1403 - DNA, 20 bp, synthetic construct
  source 1..20: mol_type = other DNA
tccgctccaggaggtccttc

SEQ ID NO: 1404 - DNA, 20 bp, synthetic construct
  source 1..20: mol_type = other DNA
agaaggacctcctggagcgg

SEQ ID NO: 1405 - DNA, 20 bp, synthetic construct
  source 1..20: mol_type = other DNA
aggacctcctggagcggagg

SEQ ID NO: 1406 - DNA, 20 bp, synthetic construct
  source 1..20: mol_type = other DNA
ggacctcctggagcggaggc

SEQ ID NO: 1407 - DNA, 20 bp, synthetic construct
  source 1..20: mol_type = other DNA
cggcccgcctccgctccagg

SEQ ID NO: 1408 - DNA, 20 bp, synthetic construct
  source 1..20: mol_type = other DNA
cctggagcggaggcgggccg

SEQ ID NO: 1409 - DNA, 20 bp, synthetic construct
  source 1..20: mol_type = other DNA
cctcggcccgcctccgctcc

SEQ ID NO: 1410 - DNA, 20 bp, synthetic construct
  source 1..20: mol_type = other DNA
ggagcggaggcgggccgagg

SEQ ID NO: 1411 - DNA, 20 bp, synthetic construct
  source 1..20: mol_type = other DNA
tctgcaataggtgtccacct

SEQ ID NO: 1412 - DNA, 20 bp, synthetic construct
  source 1..20: mol_type = other DNA
agctccccactggctttgtc

SEQ ID NO: 1413 - DNA, 20 bp, synthetic construct
  source 1..20: mol_type = other DNA
gctccccactggctttgtct

SEQ ID NO: 1414 - DNA, 20 bp, synthetic construct
  source 1..20: mol_type = other DNA
ctccccactggctttgtctg

SEQ ID NO: 1415 - DNA, 20 bp, synthetic construct
  source 1..20: mol_type = other DNA
gtccccagacaaagccagtg

SEQ ID NO: 1416 - DNA, 20 bp, synthetic construct
  source 1..20: mol_type = other DNA
tgtccccagacaaagccagt

SEQ ID NO: 1417 - DNA, 20 bp, synthetic construct
  source 1..20: mol_type = other DNA
gtgtccccagacaaagccag

SEQ ID NO: 1418 - DNA, 20 bp, synthetic construct
  source 1..20: mol_type = other DNA
gtttcctgtggcagcctaag

SEQ ID NO: 1419 - DNA, 20 bp, synthetic construct
  source 1..20: mol_type = other DNA
tttcctgtggcagcctaaga

SEQ ID NO: 1420 - DNA, 20 bp, synthetic construct
  source 1..20: mol_type = other DNA
actccctcttaggctgccac

SEQ ID NO: 1421 - DNA, 20 bp, synthetic construct
  source 1..20: mol_type = other DNA
aagaaatgacactccctctt

SEQ ID NO: 1422 - DNA, 20 bp, synthetic construct
  source 1..20: mol_type = other DNA
agggagtgtcatttcttcaa

SEQ ID NO: 1423 - DNA, 20 bp, synthetic construct
  source 1..20: mol_type = other DNA
gggagtgtcatttcttcaat

SEQ ID NO: 1424 - DNA, 20 bp, synthetic construct
  source 1..20: mol_type = other DNA
ggacagatacttctataacc

SEQ ID NO: 1425 - DNA, 20 bp, synthetic construct
  source 1..20: mol_type = other DNA
cagatacttctataaccagg

SEQ ID NO: 1426 - DNA, 20 bp, synthetic construct
  source 1..20: mol_type = other DNA
cgaagcgcacggactcctcc

SEQ ID NO: 1427 - DNA, 20 bp, synthetic construct
  source 1..20: mol_type = other DNA
ggcggcctgacgctgagtac

SEQ ID NO: 1428 - DNA, 20 bp, synthetic construct
  source 1..20: mol_type = other DNA
ctgttccagtactcagcgtc

SEQ ID NO: 1429 - DNA, 20 bp, synthetic construct
  source 1..20: mol_type = other DNA
tgagtactggaacagccaga

SEQ ID NO: 1430 - DNA, 20 bp, synthetic construct
  source 1..20: mol_type = other DNA
ccagaaggacatcctggagc

SEQ ID NO: 1431 - DNA, 20 bp, synthetic construct
  source 1..20: mol_type = other DNA
cctgctccaggatgtccttc

SEQ ID NO: 1432 - DNA, 20 bp, synthetic construct
  source 1..20: mol_type = other DNA
aggacatcctggagcaggcg

SEQ ID NO: 1433 - DNA, 20 bp, synthetic construct
  source 1..20: mol_type = other DNA
ggacatcctggagcaggcgc

SEQ ID NO: 1434 - DNA, 20 bp, synthetic construct
  source 1..20: mol_type = other DNA
cctggagcaggcgcgggccg

SEQ ID NO: 1435 - DNA, 20 bp, synthetic construct
  source 1..20: mol_type = other DNA
ccgcggcccgcgcctgctcc

SEQ ID NO: 1436 - DNA, 20 bp, synthetic construct
  source 1..20: mol_type = other DNA
ggagcaggcgcgggccgcgg

SEQ ID NO: 1437 - DNA, 20 bp, synthetic construct
  source 1..20: mol_type = other DNA
ggacctgcgctcttggaccg

SEQ ID NO: 1438 - DNA, 20 bp, synthetic construct
  source 1..20: mol_type = other DNA
tgccgtcgtaggcgtcctgc

SEQ ID NO: 1439 - DNA, 20 bp, synthetic construct
  source 1..20: mol_type = other DNA
aaggggggtggacacaacgc

SEQ ID NO: 1440 - DNA, 20 bp, synthetic construct
  source 1..20: mol_type = other DNA
ccggtggtttcggaatgacc

SEQ ID NO: 1441 - DNA, 20 bp, synthetic construct
  source 1..20: mol_type = other DNA
atacatctataaccgagagg

SEQ ID NO: 1442 - DNA, 20 bp, synthetic construct
  source 1..20: mol_type = other DNA
acgcacgcgctccgtcccat

SEQ ID NO: 1443 - DNA, 20 bp, synthetic construct
  source 1..20: mol_type = other DNA
acaccgtgtccaactccgct

SEQ ID NO: 1444 - DNA, 20 bp, synthetic construct
  source 1..20: mol_type = other DNA
ctggttgtagtagcggagca

SEQ ID NO: 1445 - DNA, 20 bp, synthetic construct
  source 1..20: mol_type = other DNA
tctggttgtagtagcggagc

SEQ ID NO: 1446 - DNA, 20 bp, synthetic construct
  source 1..20: mol_type = other DNA
cgcgctacgacagcgacgtg

SEQ ID NO: 1447 - DNA, 20 bp, synthetic construct
  source 1..20: mol_type = other DNA
aagacgcgtccataaccaag

SEQ ID NO: 1448 - DNA, 20 bp, synthetic construct
  source 1..20: mol_type = other DNA
gcgctacgacagcgacgtgg

SEQ ID NO: 1449 - DNA, 20 bp, synthetic construct
  source 1..20: mol_type = other DNA
ggaggtgtggaaatacctca

SEQ ID NO: 1450 - DNA, 20 bp, synthetic construct
  source 1..20: mol_type = other DNA
atttccacacctccgtgtcc

SEQ ID NO: 1451 - DNA, 20 bp, synthetic construct
  source 1..20: mol_type = other DNA
cgggccgggacacggaggtg

SEQ ID NO: 1452 - DNA, 20 bp, synthetic construct
  source 1..20: mol_type = other DNA
cacacctccgtgtcccggcc

SEQ ID NO: 1453 - DNA, 20 bp, synthetic construct
  source 1..20: mol_type = other DNA
gagatacatctacaaccggg

SEQ ID NO: 1454 - DNA, 20 bp, synthetic construct
  source 1..20: mol_type = other DNA
tggagagatacatctacaac

SEQ ID NO: 1455 - DNA, 20 bp, synthetic construct
  source 1..20: mol_type = other DNA
ggttgtagatgtatctctcc

SEQ ID NO: 1456 - DNA, 20 bp, synthetic construct
  source 1..20: mol_type = other DNA
ggagagatacatctacaacc

SEQ ID NO: 1457 - DNA, 20 bp, synthetic construct
  source 1..20: mol_type = other DNA
aggaatgctacgcgtttaat

SEQ ID NO: 1458 - DNA, 20 bp, synthetic construct
  source 1..20: mol_type = other DNA
caggaatgctacgcgtttaa

SEQ ID NO: 1459 - DNA, 20 bp, synthetic construct
  source 1..20: mol_type = other DNA
ttacgtgtaccagggacggc

SEQ ID NO: 1460 - DNA, 20 bp, synthetic construct
  source 1..20: mol_type = other DNA
taatgggacacagcgcttcc

SEQ ID NO: 1461 - DNA, 20 bp, synthetic construct
  source 1..20: mol_type = other DNA
atgcagacacaactacgagc

SEQ ID NO: 1462 - DNA, 20 bp, synthetic construct
  source 1..20: mol_type = other DNA
gacacaactacgagctgggc

SEQ ID NO: 1463 - DNA, 20 bp, synthetic construct
  source 1..20: mol_type = other DNA
gtgcagacacaactacgagc

SEQ ID NO: 1464 - DNA, 20 bp, synthetic construct
  source 1..20: mol_type = other DNA
agacacaactacgagctggg

SEQ ID NO: 1465 - DNA, 20 bp, synthetic construct
  source 1..20: mol_type = other DNA
tgcagacacaactacgagct

SEQ ID NO: 1466 - DNA, 20 bp, synthetic construct
  source 1..20: mol_type = other DNA
ttacgtgtaccagttacggc

SEQ ID NO: 1467 - DNA, 20 bp, synthetic construct
  source 1..20: mol_type = other DNA
agaattacgtgtaccagtta

SEQ ID NO: 1468 - DNA, 20 bp, synthetic construct
  source 1..20: mol_type = other DNA
gagatacatctacaaccggc

SEQ ID NO: 1469 - DNA, 20 bp, synthetic construct
  source 1..20: mol_type = other DNA
cgtagcattcctgccgtaac

SEQ ID NO: 1470 - DNA, 20 bp, synthetic construct
  source 1..20: mol_type = other DNA
ttacgtgcaccagttacggc

SEQ ID NO: 1471 - DNA, 20 bp, synthetic construct
  source 1..20: mol_type = other DNA
agaattacgtgcaccagtta

SEQ ID NO: 1472 - DNA, 20 bp, synthetic construct
  source 1..20: mol_type = other DNA
gacacaactacgagctggtc

SEQ ID NO: 1473 - DNA, 20 bp, synthetic construct
  source 1..20: mol_type = other DNA
ttaccttttccagggacggc